(19) United States
(12) Patent Application Publication
(10) Pub. No.: US 2026/0055084 A1
(43) Pub. Date: Feb. 26, 2026

(54) INHIBITORS OF RAF KINASES

(71) Applicant: PIERRE FABRE MEDICAMENT, Lavaur (FR)

(72) Inventors: Stephen W. KALDOR, San Diego, CA (US); Toufike KANOUNI, Rancho Santa Fe, CA (US); John TYHONAS, San Diego, CA (US); Eric MURPHY, San Marcos, CA (US)

(73) Assignee: PIERRE FABRE MEDICAMENT, Lavaur (FR)

(21) Appl. No.: 19/183,462

(22) Filed: Apr. 18, 2025

Related U.S. Application Data
(63) Continuation of application No. 17/213,036, filed on Mar. 25, 2021, now Pat. No. 12,312,336.
Application No. 17/213,036 is a continuation of application No. 17/079,152, filed on Oct. 23, 2020, now Pat. No. 11,098,031.
(60) Provisional application No. 62/925,596, filed on Oct. 24, 2019.
(60) Provisional application No. 63/044,898, filed on Jun. 26, 2020.

(51) Int. Cl. C07D 403/12; C07D 401/12; C07D 401/14; C07D 403/14; C07D 405/14; C07D 413/14

(52) U.S. Cl. CPC C07D 403/12; C07D 401/12; C07D 401/14; C07D 403/14; C07D 405/14; C07D 413/14

(57) ABSTRACT

Provided herein are inhibitors of receptor tyrosine kinase effector, RAF, pharmaceutical compositions comprising said compounds, and methods for using said compounds for the treatment of diseases.

### CROSS-REFERENCE TO RELATED APPLICATION

This application is a Continuation of copending application Ser. No. 17/213,036, filed on Mar. 25, 2021, which is a Continuation of application Ser. No. 17/079,152, filed on Oct. 23, 2020 (now U.S. Pat. No. 11,098,031 B1, issued Aug. 24, 2021), which claims priority under 35 U.S.C. § 119(e) to U.S. Provisional Application No. 63/044,898, filed on Jun. 26, 2020, and U.S. Provisional Application No. 62/925,596, filed on Oct. 24, 2019, all of which are hereby expressly incorporated by reference into the present application.

### BACKGROUND

RAF kinase functions in the Ras-Raf-MEK-ERK mitogen activated protein kinase (MAPK) pathway (also known as MAPK/ERK pathway) by phosphorylating and activating MEK. By altering the levels and activities of transcription factors, MAPK leads to altered transcription of genes that are important for the cell cycle. Deregulation of MAPK activity occurs frequently in tumors. Accordingly, therapies that target RAF kinase activity are desired for use in the treatment of cancer and other disorders characterized by aberrant MAPK/ERK pathway signaling.

### BRIEF SUMMARY OF THE INVENTION

Provided herein are inhibitors of the receptor tyrosine kinase effector Raf (RAF), pharmaceutical compositions comprising said compounds, and methods for using said compounds for the treatment of diseases.

One embodiment provides a compound, or pharmaceutically acceptable salt or solvate thereof, having the structure of Formula (I):

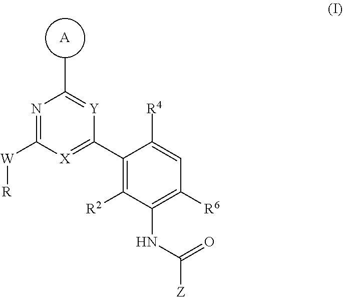

wherein,
ring A is a 5- or 6-membered optionally substituted heteroaryl, or 5- or 6-membered optionally substituted heterocyclyl;
W is NH, NR⁷, or O;
X is N, C—H, C—F, or C—CN;
Y is N, C—F, or C—H;
R is H, C1-C8 optionally substituted alkyl, (C1-C8 optionally substituted alkylene)-OPO(OH)₂, C3-C6 optionally substituted cycloalkyl, (C3-C6 optionally substituted cycloalkylene)-OPO(OH)₂, C4-C8 optionally substituted cycloalkylalkyl, (C3-C6 optionally substituted cycloalkylalkylene)-OPO(OH)₂, C3-C6 optionally substituted heterocyclyl, (C3-C6 optionally substituted heterocyclyl)-OPO(OH)₂, C3-C6 optionally substituted heterocyclylalkyl, (C3-C6 optionally substituted heterocyclylalkyl)-OPO(OH)₂, or C1-C8 optionally substituted alkyl-CO—;
R² is H, D or F;
R⁴ is halogen, optionally substituted C1-C3 alkyl, -CD₃, or optionally substituted C1-C3 alkoxy;
R⁶ is H, D, Cl or F;
R⁷ is C1-C8 optionally substituted alkyl; or R is not H, and R and R⁷ optionally join to form an optionally substituted heterocyclyl ring;
   Z is (a)

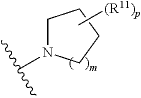

wherein m is 0, 1, 2, or 3; p is 0, 1, 2, 3, or 4; and
each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —S-alkyl, optionally substituted —SO₂ alkyl, optionally substituted C3-C6 cycloalkylalkyl, optionally substituted heterocyclyl, or optionally substituted heterocyclylalkyl; or two R¹¹ groups together form an oxo; or
(b)

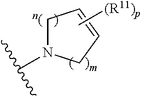

wherein m is 0, 1, 2, or 3; n is 0, 1, 2, or 3; p is 0, 1, 2, 3, or 4; and each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —S-alkyl, optionally substituted —SO₂alkyl, optionally substituted C3-C6 cycloalkylalkyl, optionally substituted heterocyclyl, or optionally substituted heterocyclylalkyl; or two R¹¹ groups together form an oxo.

One embodiment provides a pharmaceutical composition comprising a compound of Formula (I), or pharmaceutically acceptable salt or solvate thereof, and at least one pharmaceutically acceptable excipient.

One embodiment provides a method of treating a disease or disorder in a patient in need thereof comprising administering to the patient a compound of Formula (I), or pharmaceutically acceptable salt or solvate thereof. Another embodiment provides the method wherein the disease or disorder is cancer.

### INCORPORATION BY REFERENCE

All publications, patents, and patent applications mentioned in this specification are herein incorporated by reference for the specific purposes identified herein.

### DETAILED DESCRIPTION OF THE INVENTION

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

### Definitions

As used in the specification and appended claims, unless specified to the contrary, the following terms have the meaning indicated below.

“Amino” refers to the —NH₂ radical.

“Cyano” refers to the —CN radical.

“Nitro” refers to the —NO₂ radical.

“Oxa” refers to the —O— radical.

“Oxo” refers to the ═O radical.

“Thioxo” refers to the ═S radical.

“Imino” refers to the ═N—H radical.

“Oximo” refers to the ═N—OH radical.

“Hydrazino” refers to the ═N—NH₂ radical.

“Alkyl” refers to a straight or branched hydrocarbon chain radical consisting solely of carbon and hydrogen atoms, containing no unsaturation, having from one to fifteen carbon atoms (e.g., C1-C15 alkyl). In certain embodiments, an alkyl comprises one to thirteen carbon atoms (e.g., C1-C13 alkyl). In certain embodiments, an alkyl comprises one to eight carbon atoms (e.g., C1-C8 alkyl).

In other embodiments, an alkyl comprises one to five carbon atoms (e.g., C1-C5 alkyl). In other embodiments, an alkyl comprises one to four carbon atoms (e.g., C1-C4 alkyl). In other embodiments, an alkyl comprises one to three carbon atoms (e.g., C1-C3 alkyl). In other embodiments, an alkyl comprises one to two carbon atoms (e.g., C1-C2 alkyl). In other embodiments, an alkyl comprises one carbon atom (e.g., C1 alkyl). In other embodiments, an alkyl comprises five to fifteen carbon atoms (e.g., C5-C15 alkyl). In other embodiments, an alkyl comprises five to eight carbon atoms (e.g., C5-C8 alkyl). In other embodiments, an alkyl comprises two to five carbon atoms (e.g., C2-C5 alkyl). In other embodiments, an alkyl comprises three to five carbon atoms (e.g., C3-C5 alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond. Unless stated otherwise specifically in the specification, an alkyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkoxy” refers to a radical bonded through an oxygen atom of the formula —O-alkyl, where alkyl is an alkyl chain as defined above.

“Alkenyl” refers to a straight or branched hydrocarbon chain radical group consisting solely of carbon and hydrogen atoms, containing at least one carbon-carbon double bond, and having from two to twelve carbon atoms. In certain embodiments, an alkenyl comprises two to eight carbon atoms. In other embodiments, an alkenyl comprises two to four carbon atoms. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkynyl” refers to a straight or branched hydrocarbon chain radical group consisting solely of carbon and hydrogen atoms, containing at least one carbon-carbon triple bond, having from two to twelve carbon atoms. In certain embodiments, an alkynyl comprises two to eight carbon atoms. In other embodiments, an alkynyl comprises two to six carbon atoms. In other embodiments, an alkynyl comprises two to four carbon atoms. The alkynyl is attached to the rest of the molecule by a single bond, for example, ethynyl, propynyl, butynyl, pentynyl, hexynyl, and the like. Unless stated otherwise specifically in the specification, an alkynyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkylene” or “alkylene chain” refers to a straight or branched divalent hydrocarbon chain linking the rest of the molecule to a radical group, consisting solely of carbon and hydrogen, containing no unsaturation and having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group are through one carbon in the alkylene chain or through any two carbons within the chain. In certain embodiments, an alkylene comprises one to eight carbon atoms (e.g., C₁-C₈ alkylene). In other embodiments, an alkylene comprises one to five carbon atoms (e.g., C₁-C₅ alkylene). In other embodiments, an alkylene comprises one to four carbon atoms (e.g., C₁-C₄ alkylene). In other embodiments, an alkylene comprises one to three carbon atoms (e.g., C₁-C₃ alkylene). In other embodiments, an alkylene comprises one to two carbon atoms (e.g., C₁-C₂ alkylene). In other embodiments, an alkylene comprises one carbon atom (e.g., C₁ alkylene). In other embodiments, an alkylene comprises five to eight carbon atoms (e.g., C₅-C₈ alkylene). In other embodiments, an alkylene comprises two to five carbon atoms (e.g., C₂-C₅ alkylene). In other embodiments, an alkylene comprises three to five carbon atoms (e.g., C₃-C₅ alkylene). Unless stated otherwise specifically in the specification, an alkylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkenylene” or “alkenylene chain” refers to a straight or branched divalent hydrocarbon chain linking the rest of the molecule to a radical group, consisting solely of carbon and hydrogen, containing at least one carbon-carbon double bond, and having from two to twelve carbon atoms. The alkenylene chain is attached to the rest of the molecule through a single bond and to the radical group through a single bond. In certain embodiments, an alkenylene comprises two to eight carbon atoms (e.g., C₂-C₈ alkenylene). In other embodiments, an alkenylene comprises two to five carbon atoms (e.g., C₂-C₅ alkenylene). In other embodiments, an alkenylene comprises two to four carbon atoms (e.g., C₂-C₄ alkenylene). In other embodiments, an alkenylene comprises two to three carbon atoms (e.g., C₂-C₃ alkenylene). In other embodiments, an alkenylene comprises two carbon atoms (e.g., C₂ alkenylene). In other embodiments, an alkenylene comprises five to eight carbon atoms (e.g., C₅-C₈ alkenylene). In other embodiments, an alkenylene comprises three to five carbon atoms (e.g., C₃-C₅ alkenylene). Unless stated otherwise specifically in the specification, an alkenylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkynylene” or “alkynylene chain” refers to a straight or branched divalent hydrocarbon chain linking the rest of the molecule to a radical group, consisting solely of carbon and hydrogen, containing at least one carbon-carbon triple bond, and having from two to twelve carbon atoms.

The alkynylene chain is attached to the rest of the molecule through a single bond and to the radical group through a single bond. In certain embodiments, an alkynylene comprises two to eight carbon atoms (e.g., C₂-C₈ alkynylene). In other embodiments, an alkynylene comprises two to five carbon atoms (e.g., C₂-C₅ alkynylene). In other embodiments, an alkynylene comprises two to four carbon atoms (e.g., C₂-C₄ alkynylene). In other embodiments, an alkynylene comprises two to three carbon atoms (e.g., C₂-C₃ alkynylene). In other embodiments, an alkynylene comprises two carbon atoms (e.g., C₂ alkynylene). In other embodiments, an alkynylene comprises five to eight carbon atoms (e.g., C₅-C₈ alkynylene). In other embodiments, an alkynylene comprises three to five carbon atoms (e.g., C₃-C₅ alkynylene). Unless stated otherwise specifically in the specification, an alkynylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from five to eighteen carbon atoms, where at least one of the rings in the ring system is fully unsaturated, i.e., it contains a cyclic, delocalized (4n+2) 7π-electron system in accordance with the Hickel theory.

The ring system from which aryl groups are derived include, but are not limited to, groups such as benzene, fluorene, indane, indene, tetralin and naphthalene. Unless stated otherwise specifically in the specification, the term “aryl” or the prefix “ar-” (such as in “aralkyl”) is meant to include aryl radicals optionally substituted by one or more substituents independently selected from alkyl, alkenyl, alkynyl, halo, fluoroalkyl, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{b}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Aralkyl” refers to a radical of the formula —R^{c}-aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Aralkenyl” refers to a radical of the formula —R^{d}-aryl where R^{d} is an alkenylene chain as defined above. The aryl part of the aralkenyl radical is optionally substituted as described above for an aryl group. The alkenylene chain part of the aralkenyl radical is optionally substituted as defined above for an alkenylene group.

“Aralkynyl” refers to a radical of the formula —R^{e}-aryl, where R^{e} is an alkynylene chain as defined above. The aryl part of the aralkynyl radical is optionally substituted as described above for an aryl group. The alkynylene chain part of the aralkynyl radical is optionally substituted as defined above for an alkynylene chain.

“Aralkoxy” refers to a radical bonded through an oxygen atom of the formula —O—R^{e}-aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Carbocyclyl” refers to a stable non-aromatic monocyclic or polycyclic hydrocarbon radical consisting solely of carbon and hydrogen atoms, which includes fused or bridged ring systems, having from three to fifteen carbon atoms. In certain embodiments, a carbocyclyl comprises three to ten carbon atoms. In other embodiments, a carbocyclyl comprises five to seven carbon atoms.

The carbocyclyl is attached to the rest of the molecule by a single bond. Carbocyclyl is saturated (i.e., containing single C—C bonds only) or unsaturated (i.e., containing one or more double bonds or triple bonds). A fully saturated carbocyclyl radical is also referred to as “cycloalkyl.” Examples of monocyclic cycloalkyls include, e.g., cyclopropyl, cyclobutyl, cyclopentyl, cyclohexyl, cycloheptyl, and cyclooctyl. An unsaturated carbocyclyl is also referred to as “cycloalkenyl.” Examples of monocyclic cycloalkenyls include, e.g., cyclopentenyl, cyclohexenyl, cycloheptenyl, and cyclooctenyl. Polycyclic carbocyclyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Unless otherwise stated specifically in the specification, the term “carbocyclyl” is meant to include carbocyclyl radicals that are optionally substituted by one or more substituents independently selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Carbocyclylalkyl” refers to a radical of the formula —R^{c}-carbocyclyl where R^{c} is an alkylene chain as defined above. The alkylene chain and the carbocyclyl radical is optionally substituted as defined above.

“Carbocyclylalkynyl” refers to a radical of the formula —R^{c}-carbocyclyl where R^{c} is an alkynylene chain as defined above. The alkynylene chain and the carbocyclyl radical is optionally substituted as defined above.

“Carbocyclylalkoxy” refers to a radical bonded through an oxygen atom of the formula —O—R^{c}-carbocyclyl where R^{c} is an alkylene chain as defined above. The alkylene chain and the carbocyclyl radical is optionally substituted as defined above.

As used herein, “carboxylic acid bioisostere” refers to a functional group or moiety that exhibits similar physical, biological and/or chemical properties as a carboxylic acid moiety. Examples of carboxylic acid bioisosteres include, but are not limited to,

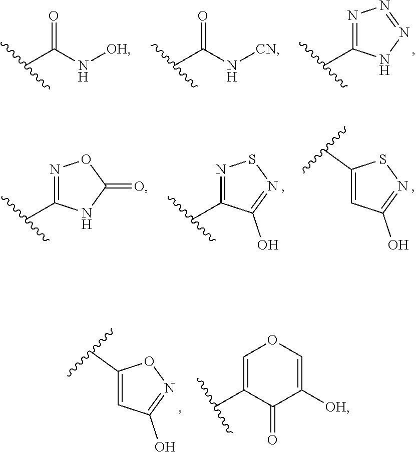

and the like.

“Halo” or “halogen” refers to bromo, chloro, fluoro or iodo substituents.

“Fluoroalkyl” refers to an alkyl radical, as defined above, that is substituted by one or more fluoro radicals, as defined above, for example, trifluoromethyl, difluoromethyl, fluoromethyl, 2,2,2-trifluoroethyl, 1-fluoromethyl-2-fluoroethyl, and the like. In some embodiments, the alkyl part of the fluoroalkyl radical is optionally substituted as defined above for an alkyl group.

“Heterocyclyl” refers to a stable 3- to 18-membered non-aromatic ring radical that comprises two to twelve carbon atoms and from one to six heteroatoms selected from nitrogen, oxygen and sulfur. Unless stated otherwise specifically in the specification, the heterocyclyl radical is a monocyclic, bicyclic, tricyclic or tetracyclic ring system, which optionally includes fused or bridged ring systems. The heteroatoms in the heterocyclyl radical are optionally oxidized. One or more nitrogen atoms, if present, are optionally quaternized. The heterocyclyl radical is partially or fully saturated. The heterocyclyl is attached to the rest of the molecule through any atom of the ring(s). Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, the term “heterocyclyl” is meant to include heterocyclyl radicals as defined above that are optionally substituted by one or more substituents selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{c}—O—R^{b}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heterocyclyl” or “N-attached heterocyclyl” refers to a heterocyclyl radical as defined above containing at least one nitrogen and where the point of attachment of the heterocyclyl radical to the rest of the molecule is through a nitrogen atom in the heterocyclyl radical. An N-heterocyclyl radical is optionally substituted as described above for heterocyclyl radicals. Examples of such N-heterocyclyl radicals include, but are not limited to, 1-morpholinyl, 1-piperidinyl, 1-piperazinyl, 1-pyrrolidinyl, pyrazolidinyl, imidazolinyl, and imidazolidinyl.

“C-heterocyclyl” or “C-attached heterocyclyl” refers to a heterocyclyl radical as defined above containing at least one heteroatom and where the point of attachment of the heterocyclyl radical to the rest of the molecule is through a carbon atom in the heterocyclyl radical. A C-heterocyclyl radical is optionally substituted as described above for heterocyclyl radicals. Examples of such C-heterocyclyl radicals include, but are not limited to, 2-morpholinyl, 2- or 3- or 4-piperidinyl, 2-piperazinyl, 2- or 3-pyrrolidinyl, and the like.

“Heterocyclylalkyl” refers to a radical of the formula —R^{e}-heterocyclyl where R^{e} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkyl radical is optionally substituted as defined above for an alkylene chain. The heterocyclyl part of the heterocyclylalkyl radical is optionally substituted as defined above for a heterocyclyl group.

“Heterocyclylalkoxy” refers to a radical bonded through an oxygen atom of the formula —O—R^{c}-heterocyclyl where R^{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkoxy radical is optionally substituted as defined above for an alkylene chain. The heterocyclyl part of the heterocyclylalkoxy radical is optionally substituted as defined above for a heterocyclyl group.

“Heteroaryl” refers to a radical derived from a 3- to 18-membered aromatic ring radical that comprises two to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system is fully unsaturated, i.e., it contains a cyclic, delocalized (4n+2) 7π-electron system in accordance with the Hickel theory. Heteroaryl includes fused or bridged ring systems. The heteroatom(s) in the heteroaryl radical is optionally oxidized. One or more nitrogen atoms, if present, are optionally quaternized.

The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pridinyl, and thiophenyl (i.e. thienyl). Unless stated otherwise specifically in the specification, the term “heteroaryl” is meant to include heteroaryl radicals as defined above which are optionally substituted by one or more substituents selected from alkyl, alkenyl, alkynyl, halo, fluoroalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heteroaryl” refers to a heteroaryl radical as defined above containing at least one nitrogen and where the point of attachment of the heteroaryl radical to the rest of the molecule is through a nitrogen atom in the heteroaryl radical. An N-heteroaryl radical is optionally substituted as described above for heteroaryl radicals.

“C-heteroaryl” refers to a heteroaryl radical as defined above and where the point of attachment of the heteroaryl radical to the rest of the molecule is through a carbon atom in the heteroaryl radical. A C-heteroaryl radical is optionally substituted as described above for heteroaryl radicals.

“Heteroarylalkyl” refers to a radical of the formula —R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkyl radical is optionally substituted as defined above for an alkylene chain. The heteroaryl part of the heteroarylalkyl radical is optionally substituted as defined above for a heteroaryl group.

“Heteroarylalkoxy” refers to a radical bonded through an oxygen atom of the formula —O—R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkoxy radical is optionally substituted as defined above for an alkylene chain. The heteroaryl part of the heteroarylalkoxy radical is optionally substituted as defined above for a heteroaryl group.

The compounds disclosed herein, in some embodiments, contain one or more asymmetric centers and thus give rise to enantiomers, diastereomers, and other stereoisomeric forms that are defined, in terms of absolute stereochemistry, as (R)- or (S)-. Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans.) Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a benzene ring.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

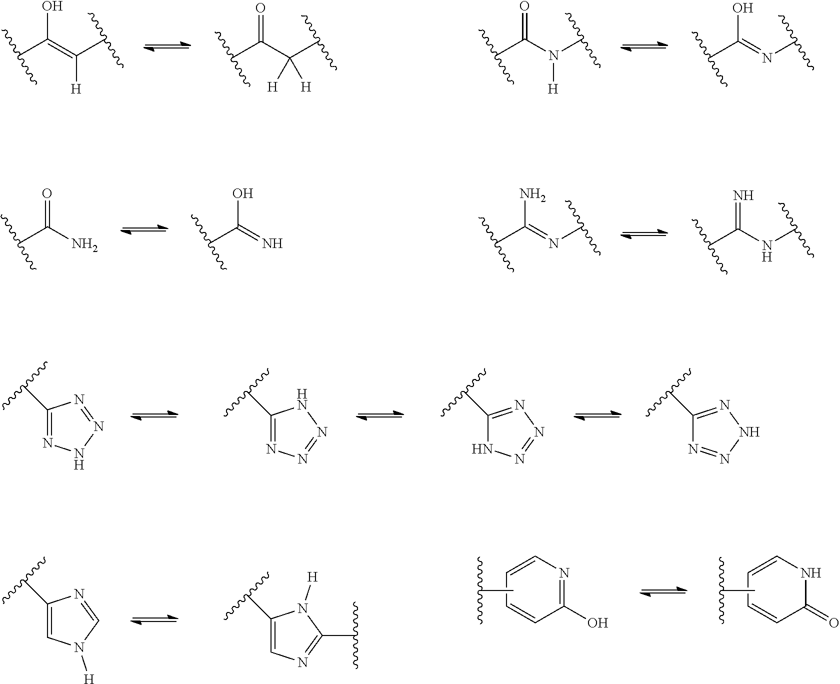

The compounds disclosed herein, in some embodiments, are used in different enriched isotopic forms, e.g., enriched in the content of ²H, ³H, ¹¹C, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, structures depicted herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (²H), tritium (³H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, ¹²⁵I are all contemplated. In some embodiments, isotopic substitution with ¹⁸F is contemplated. All isotopic variations of the compounds of the present invention, whether radioactive or not, are encompassed within the scope of the present invention.

In certain embodiments, the compounds disclosed herein have some or all of the iH atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [Curr., Pharm. Des., 2000; 6(10)]2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Deuterium-transfer reagents suitable for use in nucleophilic substitution reactions, such as iodomethane-d₃ (CD₃I), are readily available and may be employed to transfer a deuterium-substituted carbon atom under nucleophilic substitution reaction conditions to the reaction substrate. The use of CD₃I is illustrated, by way of example only, in the reaction schemes below.

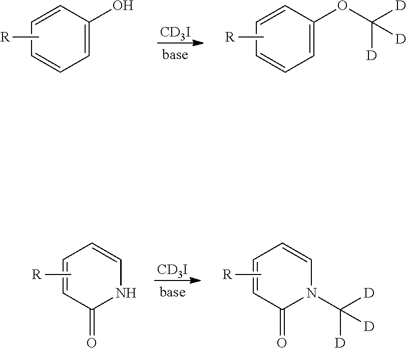

Deuterium-transfer reagents, such as lithium aluminum deuteride (LiAlD₄), are employed to transfer deuterium under reducing conditions to the reaction substrate. The use of LiAlD₄ is illustrated, by way of example only, in the reaction schemes below.

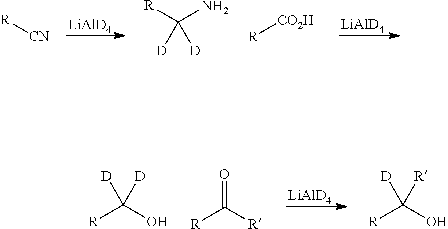

Deuterium gas and palladium catalyst are employed to reduce unsaturated carbon-carbon linkages and to perform a reductive substitution of aryl carbon-halogen bonds as illustrated, by way of example only, in the reaction schemes below.

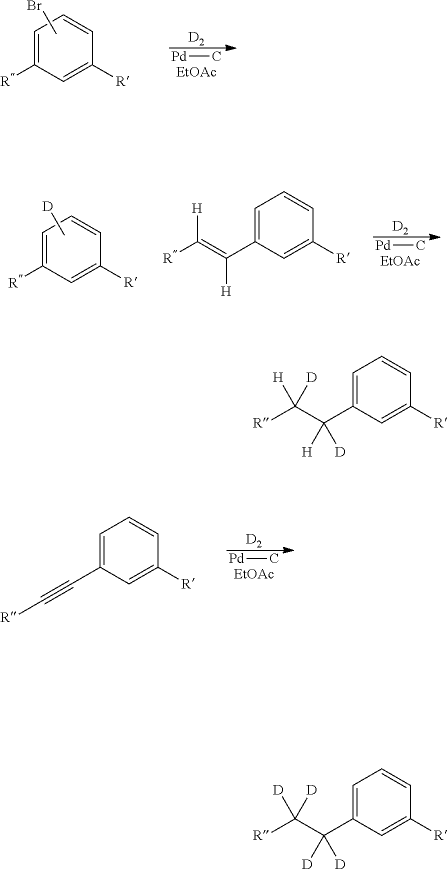

In one embodiment, the compounds disclosed herein contain one deuterium atom. In another embodiment, the compounds disclosed herein contain two deuterium atoms. In another embodiment, the compounds disclosed herein contain three deuterium atoms. In another embodiment, the compounds disclosed herein contain four deuterium atoms. In another embodiment, the compounds disclosed herein contain five deuterium atoms. In another embodiment, the compounds disclosed herein contain six deuterium atoms. In another embodiment, the compounds disclosed herein contain more than six deuterium atoms. In another embodiment, the compound disclosed herein is fully substituted with deuterium atoms and contains no non-exchangeable ¹H hydrogen atoms. In one embodiment, the level of deuterium incorporation is determined by synthetic methods in which a deuterated synthetic building block is used as a starting material.

“Pharmaceutically acceptable salt” includes both acid and base addition salts. A pharmaceutically acceptable salt of any one of the heteroaromatic RAF inhibitory compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997)). Acid addition salts of basic compounds are, in some embodiments, prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts are, in some embodiments, formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

“Pharmaceutically acceptable solvate” refers to a composition of matter that is the solvent addition form. In some embodiments, solvates contain either stoichiometric or non-stoichiometric amounts of a solvent, and are formed during the process of making with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. The compounds provided herein optionally exist in either unsolvated as well as solvated forms. The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

As used herein, “treatment” or “treating,” or “palliating” or “ameliorating” are used interchangeably. These terms refer to an approach for obtaining beneficial or desired results including but not limited to therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is still afflicted with the underlying disorder. For prophylactic benefit, the compositions are, in some embodiments, administered to a patient at risk of developing a particular disease, or to a patient reporting one or more of the physiological symptoms of a disease, even though a diagnosis of this disease has not been made.

### The RAF Family of Kinases

The RAF kinases are a family of serine/thronine protein kinases constitute core components of the RAS-RAF-MEK-ERK mitogen activated protein kinase (MAPK) signalling cascade (also known as the MAPK/ERK pathway), a pathway that mediates signals from cell surface receptors to the nucleus to regulate cell growth, differentiation and survival. The RAF proteins are related to retroviral oncogenes and are structurally conserved from metazoans to mammals, as is the MAPK/ERK pathway. Their dysregulation leads to uncontrolled cellular proliferation, survival and dedifferentiation. Consequently, RAF kinases are altered or inappropriately activated in a majority of cancers.

The MAPK/ERK signalling pathway is a network of proteins in the cell that communicates a signal from a receptor on the surface of the cell to the DNA in the nucleus of the cell. The signal starts when a signaling molecule binds to the receptor on the cell surface and ends when the DNA in the nucleus expresses a protein and produces some change in the cell, such as cell division. The pathway includes many proteins, which communicate by adding phosphate groups to a neighboring protein, which acts as a molecular “on” or “off” switch, and overall the pathway can be divided into 3 steps: (i) Ras activation, (ii) a kinase signal transduction cascade, and (iii) regulation of translation and transcription. Briefly, an extracellular mitogen or a signaling molecule binds to the membrane receptor. This allows Ras (a small GTPase) to swap its GDP for a GTP and become active. Activated Ras activates the protein kinase activity of RAF kinase. RAF kinase phosphorylates and activates MEK (MEK1 and MEK2). MEK then phosphorylates and activates a MAPK (also known as ERK). MAPK activation regulates activities of several transcription factors and also alters the translation of mRNA to proteins. By altering the levels and activities of transcription factors, MAPK leads to altered transcription of genes that are important for the cell cycle.

There are three known mammalian RAF isoforms: C-RAF (also known as RAF-1, or c-RAF-1), B-RAF, and A-RAF. All RAF kinases share a common modular structure consisting of 3 conserved regions (CR1, CR2, and CR3) with distinct functions. CR1 contains (i) a Ras-binding domain (RBD), which is necessary for the interaction with Ras and with membrane phospholipids required for membrane recruitment, and (ii) a cysteine-rich domain (CRD), which is a secondary Ras-binding site and also necessary for the interaction of CR1 with the kinase domain for RAF autoinhibition. CR2 contains important inhibitory phosphorylation sites participating in the negative regulation of Ras binding and RAF activation. CR3 features the kinase domain, including the activation segment, whose phosphorylation is crucial for kinase activation.

Functionally, the RAF structure can be split into a regulatory N-terminal region, containing the RBD, which is critical for activation as well as inhibitory phosphorylation sites, and a catalytic C-terminal region, which includes phosphorylation sites necessary for the kinase activation. The regulatory domain restrains the activity of the kinase domain, and its removal results in constitutive oncogenic activation. However, the activity of the isolated C-RAF kinase domain is subjected to further regulation and can be stimulated by phorbol esters, v-Src, and phosphorylation.

The common and key step in the activation of all 3 RAF kinase isoforms is membrane recruitment by a Ras family protein. The RAF kinases are located in the cytosol in their inactive state when bound to 14-3-3 proteins. In the presence of active Ras, they translocate to the plasma membrane. Membrane translocation triggers further activation events, such as the binding of PP2A to dephosphorylate the inhibitory pS259 site in RAF-1 (and presumably the corresponding sites in A-RAF and B-RAF) and the co-localization with the kinases responsible for the multiple activating phosphorylations. The sequences forming the binding interface are well conserved in the RAF as well as Ras family indicating that several members of the Ras family have the ability to bind RAF kinases. H-Ras, N-Ras, and K-Ras stimulate all 3 RAF isoforms and are the only Ras proteins that activate B-RAF. In contrast, A-RAF is also activated by R-Ras3, while C-RAF responds weakly to R-Ras3, Rit, and TC21 as well. But, all RAF kinases share MEK1/2 kinases as substrates. MEK1/2 in turn activate ERK1/2, and this pathway regulates many cellular functions such as cell proliferation, differentiation, migration, or apoptosis.

### C-RAF

C-RAF was first to be identified and is a ubiquitously expressed isoform. In humans, C-RAF is encoded by the RAF1 gene. C-RAF also has a known splice variant preferentially expressed in the muscle and brain. C-RAF plays a critical role in mediating the cellular effects of growth factor signals. In the inactive state, C-RAF exists in a closed conformation in which the N-terminal regulatory region folds over and occludes the catalytic region. This conformation is stabilized by a 14-3-3 dimer binding to an N-terminal site, phospho-S259 (pS259), and a C-terminal site, pS621. Dephosphorylation of pS259 at the cell membrane by specific phosphatases (PP2A, PP1) releases 14-3-3 from its N-terminal binding site in C-RAF, thereby allowing conformational changes to occur that unmask the RBD and CRD domains in the CR1 region to enable Ras binding and membrane recruitment.

### B-RAF

B-RAF is encoded in humans by the BRAF gene, also known as proto-oncogene B-RAF and v-RAF murine sarcoma viral oncogene homolog B. Alternative splicing gives rise to multiple B-RAF isoforms which are differentially expressed in various tissues. Whereas activation of A-RAF and C-RAF requires both phosphorylation and dephosphorylation of certain residues, as well as binding to other proteins, B-RAF becomes activated immediately upon translocation to the plasma membrane. B-RAF exhibits higher basal kinase activity than A-RAF and C-RAF. B-RAF requires Ras and 14-3-3 binding for its activation and is inhibited or activated by PKA depending on the levels of 14-3-3 expression, which need to be high for permitting activation. B-RAF activity is also regulated by splicing. B-RAF isoforms containing exon 8b are more phosphorylated on the inhibitory S365 site, leading to an increased interaction with 14-3-3 and strengthening the inhibitory interaction between N-terminal regulatory domain and kinase domain, altogether resulting in lower kinase activity.

### A-RAF

Serine/threonine-protein kinase A-RAF or A-RAF is an enzyme encoded by the ARAF gene in humans. There are 2 known splice isoforms of A-RAF-DA-RAF1 and D-RAF2. They lack the kinase domain and act as dominant inhibitory mutants of Ras and ARF GTPases. DA-RAF1 is a positive regulator of myogenic differentiation by mediating the inhibition of the ERK pathway required for differentiation. There are several ways A-RAF is different from the other RAF kinases. A-RAF is the only steroid hormone-regulated Raf isoform. In addition, the A-RAFprotein has amino acid substitutions in a negatively charged region upstream of the kinase domain (N-region), which contributes to its low basal activity. A-RAF is also only weakly activated by oncogenic H-Ras and Src and also displays low kinase activity towards MEK (the lowest kinase activity towards MEK proteins in the Raf kinase family). In addition to phosphorylating MEK, A-RAF also inhibits MST2, a tumor suppressor and pro-apoptotic kinase not found in the MAPK pathway. By inhibiting MST2, A-RAF prevents apoptosis from occurring. However, this inhibition is only occurs when the splice factor heterogenous nuclear ribonucleoprotein H (hnRNP H) maintains the expression of a full-length A-RAF protein. Tumorous cells often overexpress hnRNP H which leads to full-length expression of A-Raf which then inhibits apoptosis, allowing cancerous cells that should be destroyed to stay alive. A-RAF also binds to pyruvate kinase M2 (PKM2), again outside the MAPK pathway. PKM2 is an isozyme of pyruvate kinase that is responsible for the Warburg effect in cancer cells. A-RAF upregulates the activity of PKM2 by promoting a conformational change in PKM2. This causes PKM2 to transition from its low-activity dimeric form to a highly active tetrameric form. This causes more glucose carbons to be converted to pyruvate and lactate, producing energy for the cell, linking A-Raf to energy metabolism regulation and cell transformation, both of which are very important in tumorigenesis.

### RAF Kinase Inhibitors

Aberrant activation of the MAPK/ERK pathway is frequently found in various cancers and is a target for cancer therapeutics. In particular, B-RAF has emerged as one of the most attractive molecular targets for cancer therapeutics because somatic mutations of B-RAF have frequently been found in human tumors. Approximately 20% of all cancer samples tested to date harbor mutations in B-RAF. B-RAF-V600E, a missense mutation in the kinase domain generated by the substitution of glutamic acid with valine at position 600 is the most common B-RAF mutation. C-RAF is mutated in ˜1% of the various tumor types tested and the rate of mutations in A-RAF is even lower. B-RAF and C-RAF form both homo- and heterodimers as part of their activation mechanism and A-RAF stabilizes the B-RAF:C-RAF complexes to sustain signaling efficiency. Also, it is C-RAF, not B-RAF, that transmits signals from oncogenic RAS to MEK. Therefore, in different contexts, each of the RAF isoforms act as a potential therapeutic target.

Sorafenib was the first RAF inhibitor to enter clinical trials. Sorafenib is a broad specificity drug that inhibits additional kinases, including vascular endothelial growth factor receptor family (VEGFR-2 and VEGFR-3), platelet-derived growth factor receptor family (PDGFR-b and KIT) and FLT3. Clinical trials showed no correlation between the clinical responses with B-RAF mutation status, indicating it is a poor inhibitor of B-RAF. This led to the development of a new generation of B-RAF inhibitors, including, but not limited to vemurafenib, SB-590885, and dabrafenib (GSK2118436). Although the initial results of the clinical studies in B-RAF-mutant melanoma were encouraging, as clinical testing began in other B-RAF-mutated cancers (such as thyroid and colorectal cancers) it became apparent that tumors of different cell types harboring B-RAF mutations responded differently to selective B-RAF inhibition. Moreover, the existence of both primary and secondary resistance to RAF inhibition poses as one of the greatest challenges to the progress of RAF kinase inhibitor therapy. The mechanisms of resistance fall into two broad categories. Intrinsic/primary resistance is displayed by approximately 50% of patients. The other 50% of the patients initially respond (>30% tumor shrinkage) to RAF inhibitor but subsequently develop progressive disease associated with acquired/secondary resistance to RAF inhibitor. These two categories are not mutually exclusive because nearly all responders have remaining disease and, thus, may display intrinsic resistance. The determinants of primary RAF inhibitor resistance seem to vary with tumor type, with alteration in RTK signaling also being involved. Potential mechanisms of secondary B-RAF inhibitor resistance include, but are not limited to, reactivation of ERK1/2 pathways, upregulation of RTK signaling, the upregulation of receptor tyrosine kinases, mutations in RAS, and upregulation of COT. B-Raf alternative splicing and amplification of B-RAF-V600E have also been implicated in ˜30 and 20% of patients, respectively. Moreover, RAF kinase inhibitors cause paradoxical activation of the MAPK pathway, which, in some instances, leads to the development of secondary RAS mutation-driven malignancies. As such, there is a need in the field for new RAF kinase inhibitors that overcome the existing pitfalls and challenges posed by the current inhibitors.

### Heteroaromatic RAF Inhibitory Compounds

In one aspect, provided herein is a heteroaromatic RAF inhibitory compound.

One embodiment provides a compound, or pharmaceutically acceptable salt or solvate thereof, having the structure of Formula (I):

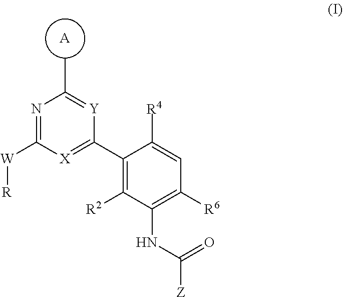

wherein,
ring A is a 5- or 6-membered optionally substituted heteroaryl, or 5- or 6-membered optionally substituted heterocyclyl;
W is NH, NR⁷, or O;
X is N, C—H, C—F, or C—CN;
Y is N, C—F, or C—H;
R is H, C1-C8 optionally substituted alkyl, (C1-C8 optionally substituted alkylene)-OPO(OH)₂, C3-C6 optionally substituted cycloalkyl, (C3-C6 optionally substituted cycloalkylene)-OPO(OH)₂, C4-C8 optionally substituted cycloalkylalkyl, (C3-C6 optionally substituted cycloalkylalkylene)-OPO(OH)₂, C3-C6 optionally substituted heterocyclyl, (C3-C6 optionally substituted heterocyclyl)-OPO(OH)₂, C3-C6 optionally substituted heterocyclylalkyl, (C3-C6 optionally substituted heterocyclylalkyl)-OPO(OH)₂, or C1-C8 optionally substituted alkyl-CO—;
R² is H, D or F;
R⁴ is halogen, optionally substituted C1-C3 alkyl, -CD₃, or optionally substituted C1-C3 alkoxy;
R⁶ is H, D, Cl or F;
R⁷ is C1-C8 optionally substituted alkyl; or R is not H, and R and R⁷ optionally join to form an optionally substituted heterocyclyl ring;
Z is:
(a)

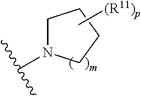

wherein m is 0, 1, 2, or 3; p is 0, 1, 2, 3, or 4; and each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —S-alkyl, optionally substituted —SO₂alkyl, optionally substituted C3-C6 cycloalkylalkyl, optionally substituted heterocyclyl, or optionally substituted heterocyclylalkyl; or two R¹¹ groups together form an oxo; or
(b)

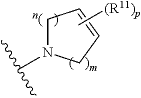

wherein m is 0, 1, 2, or 3; n is 0, 1, 2, or 3; p is 0, 1, 2, 3, or 4; and each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —S-alkyl, optionally substituted —SO₂alkyl, optionally substituted C3-C6 cycloalkylalkyl, optionally substituted heterocyclyl, or optionally substituted heterocyclylalkyl; or two R¹¹ groups together form an oxo.

Another embodiment provides the compound of Formula (I), or pharmaceutically acceptable salt or solvate thereof, wherein ring A is a 5- or 6-membered optionally substituted heteroaryl.

Another embodiment provides the compound of Formula (I), or pharmaceutically acceptable salt or solvate thereof, wherein ring A is an optionally substituted pyridine or optionally substituted pyrrazole. Another embodiment provides the compound of Formula (I), or pharmaceutically acceptable salt or solvate thereof, wherein ring A is an optionally substituted 4-pyridinyl or optionally substituted 3-pyrrazole.

Another embodiment provides the compound of Formula (I), or pharmaceutically acceptable salt or solvate thereof, wherein ring A is a 5- or 6-membered optionally substituted heterocyclyl.

Another embodiment provides the compound of Formula (I), or pharmaceutically acceptable salt or solvate thereof, wherein ring A is an optionally substituted dihydropyran, optionally substituted tetrahydropyran, optionally substituted dihydrofuran, or optionally substituted tetrahydrofuran. Another embodiment provides the compound of Formula (I), or pharmaceutically acceptable salt or solvate thereof, wherein ring A is an optionally substituted dihydro-2H-pyran-4-yl, optionally substituted tetrahydropyran-4-yl, optionally substituted 2,5-dihydrofuran-3-yl, optionally substituted tetrahydrofuran-3-yl, 3-oxabicyclo[3.1.0]hexan-1-yl, or 3-oxabicyclo[4.1.0]heptan-6-yl.

Another embodiment provides the compound of Formula (I), or pharmaceutically acceptable salt or solvate thereof, wherein ring A is an optionally substituted morpholine.

Another embodiment provides the compound of Formula (I), or pharmaceutically acceptable salt or solvate thereof, having the structure of Formula (II):

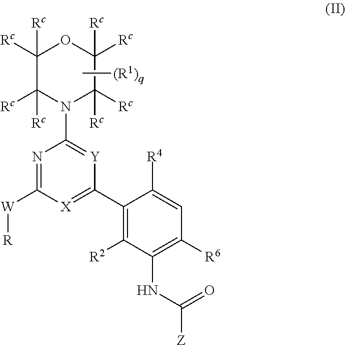

wherein,
W is NH, NR⁷, or O;
X is N, C—H, C—F, or C—CN;
Y is N, C—F, or C—H;
R is H, C1-C8 optionally substituted alkyl, (C1-C8 optionally substituted alkylene)-OPO(OH)₂, C3-C6 optionally substituted cycloalkyl, (C3-C6 optionally substituted cycloalkylene)-OPO(OH)₂, C4-C8 optionally substituted cycloalkylalkyl, (C3-C6 optionally substituted cycloalkylalkylene)-OPO(OH)₂, C3-C6 optionally substituted heterocyclyl, (C3-C6 optionally substituted heterocyclyl)-OPO(OH)₂, C3-C6 optionally substituted heterocyclylalkyl, (C3-C6 optionally substituted heterocyclylalkyl)-OPO(OH)₂, or C1-C8 optionally substituted alkyl-CO—;
R¹ is C1-C3 optionally substituted alkyl, and q is 0, 1, or 2;
R² is H, D or F;
R⁴ is halogen, optionally substituted C1-C3 alkyl, -CD₃, or optionally substituted C1-C3 alkoxy;
R⁶ is H, D, Cl or F;
R⁷ is C1-C8 optionally substituted alkyl; or R is not H, and R and R⁷ optionally join to form an optionally substituted heterocyclyl ring;
R^{c} is H or D;
Z is (a)

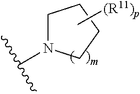

wherein m is 0, 1, 2, or 3; p is 0, 1, 2, 3, or 4; and each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —S-alkyl, optionally substituted —SO₂alkyl, optionally substituted C3-C6 cycloalkylalkyl, optionally substituted heterocyclyl, or optionally substituted heterocyclylalkyl; or two R¹¹ groups together form an oxo; or
(b)

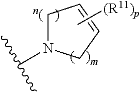

wherein n is 1, 2, or 3; m is 1, 2, or 3; p is 0, 1, 2, 3, or 4; and each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —S-alkyl, optionally substituted —SO₂alkyl, optionally substituted C3-C6 cycloalkylalkyl, optionally substituted heterocyclyl, or optionally substituted heterocyclylalkyl; or two R¹¹ groups together form an oxo.

Another embodiment provides the compound of Formula (I), or pharmaceutically acceptable salt or solvate thereof, having the structure of Formula (III):

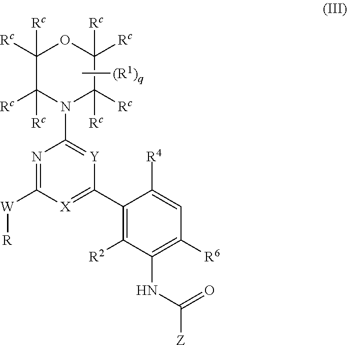

wherein,
W is NH or 0;
X is N, C—H, or C—CN;
Y is N or C—H;
R is C1-C8 optionally substituted alkyl, —(C1-C8 optionally substituted alkylene)-OPO(OH)₂, C3-C6 optionally substituted cycloalkyl, —(C3-C6 optionally substituted cycloalkylene)-OPO(OH)₂, C4-C6 optionally substituted cycloalkylalkyl, —(C3-C6 optionally substituted cycloalkylalkylene)-OPO(OH)₂, C3-C6 optionally substituted heterocyclyl, —(C3-C6 optionally substituted heterocyclyl)-OPO(OH)₂, C3-C6 optionally substituted heterocyclylalkyl, —(C3-C6 optionally substituted heterocyclylalkyl)-OPO(OH)₂;
R¹ is C1-C3 optionally substituted alkyl, and q is 0, 1, or 2;
R² is H, D or F;
R⁴ is halogen, optionally substituted C1-C3 alkyl, -CD₃, or optionally substituted C1-C3 alkoxy;
R⁶ is H, D, Cl or F;
R^{c} is H or D;
Z is

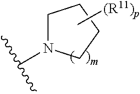

wherein m is 0, 1, 2, or 3; p is 0, 1, 2, 3, or 4; and each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —S-alkyl, optionally substituted —SO₂alkyl, optionally substituted C3-C6 cycloalkylalkyl, optionally substituted heterocyclyl, or optionally substituted heterocyclylalkyl; or two R¹¹ groups together form an oxo.

Another embodiment provides the compound of Formula (I)-(III), or pharmaceutically acceptable salt or solvate thereof, wherein W is NH.

Another embodiment provides the compound of Formula (I) or (II), or pharmaceutically acceptable salt or solvate thereof, wherein W is NR⁷. Another embodiment provides the compound of Formula (I) or (II), or pharmaceutically acceptable salt or solvate thereof, wherein R⁷ is optionally substituted C1 alkyl.

Another embodiment provides the compound of Formula (I) or (II), or pharmaceutically acceptable salt or solvate thereof, wherein W is NR⁷, R is not H, and R and R⁷ join to form an optionally substituted heterocyclyl ring. Another embodiment provides the compound of Formula (I) or (II), or pharmaceutically acceptable salt or solvate thereof, wherein the optionally substituted heterocyclyl ring is a 1-aztidinyl, 1-pyrrolidinyl, or 1-piperidinyl ring. Another embodiment provides the compound of Formula (I) or (II), or pharmaceutically acceptable salt or solvate thereof, wherein the optionally substituted heterocyclyl ring is substituted with at least one substituent selected from —OH, halogen, optionally substituted C1-C6 alkyl, or optionally substituted C1-C6 alkoxy.

Another embodiment provides the compound of Formula (I)-(III), or pharmaceutically acceptable salt or solvate thereof, wherein R is C1-C8 optionally substituted alkyl, C3-C6 optionally substituted cycloalkyl, C4-C8 optionally substituted cycloalkylalkyl, C3-C6 optionally substituted heterocyclyl, or C3-C6 optionally substituted heterocyclylalkyl.

Another embodiment provides the compound of Formula (I)-(III), or pharmaceutically acceptable salt or solvate thereof, wherein R is C1-C8 optionally substituted alkyl, C3-C6 optionally substituted cycloalkyl, C4-C6 optionally substituted cycloalkylalkyl, C3-C6 optionally substituted heterocyclyl, or C3-C6 optionally substituted heterocyclylalkyl. Another embodiment provides the compound of Formula (I)-(III), or pharmaceutically acceptable salt or solvate thereof, wherein R is C1-C8 optionally substituted alkyl. Another embodiment provides the compound of Formula (I)-(III), or pharmaceutically acceptable salt or solvate thereof, wherein R is C1-C6 optionally substituted alkyl. Another embodiment provides the compound of Formula (I)-(III), or pharmaceutically acceptable salt or solvate thereof, wherein R is C1-C4 optionally substituted alkyl. Another embodiment provides the compound of Formula (I)-(III), or pharmaceutically acceptable salt or solvate thereof, wherein R is C1-C3 optionally substituted alkyl. Another embodiment provides the compound of Formula (I)-(III), or pharmaceutically acceptable salt or solvate thereof, wherein R is C1-C2 optionally substituted alkyl. Another embodiment provides the compound of Formula (I)-(III), or pharmaceutically acceptable salt or solvate thereof, wherein R is C1-C6 optionally substituted alkyl and the alkyl is substituted with at least one hydroxyl.

Another embodiment provides the compound of Formula (I) or (II), or pharmaceutically acceptable salt or solvate thereof, wherein R is H.

Another embodiment provides the compound of Formula (I) or (II), or pharmaceutically acceptable salt or solvate thereof, wherein R is C1-C8 optionally substituted alkyl-CO—.

Another embodiment provides the compound of Formula (I)-(III), or pharmaceutically acceptable salt or solvate thereof, wherein R is C3-C6 optionally substituted cycloalkyl.

Another embodiment provides the compound of Formula (I)-(III), or pharmaceutically acceptable salt or solvate thereof, wherein R is C4-C6 optionally substituted cycloalkylalkyl.

Another embodiment provides the compound of Formula (I)-(III), or pharmaceutically acceptable salt or solvate thereof, wherein R is C3-C6 optionally substituted heterocyclyl.

Another embodiment provides the compound of Formula (I)-(III), or pharmaceutically acceptable salt or solvate thereof, wherein R is C3-C6 optionally substituted heterocyclylalkyl.

Another embodiment provides the compound of Formula (II)-(III), or pharmaceutically acceptable salt or solvate thereof, wherein R¹ is C1 optionally substituted alkyl. Another embodiment provides the compound of Formula (II)-(III), or pharmaceutically acceptable salt or solvate thereof, wherein q is 0. Another embodiment provides the compound of Formula (II)-(III), or pharmaceutically acceptable salt or solvate thereof, wherein q is 1.

Another embodiment provides the compound of Formula (I)-(III), or pharmaceutically acceptable salt or solvate thereof, wherein Z is

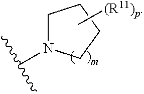

Another embodiment provides the compound of Formula (I)-(III), or pharmaceutically acceptable salt or solvate thereof, wherein m is 0. Another embodiment provides the compound of Formula (I)-(III), or pharmaceutically acceptable salt or solvate thereof, wherein m is 1. Another embodiment provides the compound of Formula (I)-(III), or pharmaceutically acceptable salt or solvate thereof, wherein m is 2. Another embodiment provides the compound of Formula (I)-(III), or pharmaceutically acceptable salt or solvate thereof, wherein p is 1. Another embodiment provides the compound of Formula (I)-(III), or pharmaceutically acceptable salt or solvate thereof, wherein p is 2.

Another embodiment provides the compound of Formula (I)-(III), or pharmaceutically acceptable salt or solvate thereof, wherein R¹¹ is optionally substituted C1-C6 alkyl. Another embodiment provides the compound of Formula (I)-(III), or pharmaceutically acceptable salt or solvate thereof, wherein R¹¹ is optionally substituted C1-C4 alkyl. Another embodiment provides the compound of Formula (I)-(III), or pharmaceutically acceptable salt or solvate thereof, wherein R¹¹ is optionally substituted C1-C3 alkyl. Another embodiment provides the compound of Formula (I)-(III), or pharmaceutically acceptable salt or solvate thereof, wherein R¹¹ is optionally substituted C1-C2 alkyl. Another embodiment provides the compound of Formula (I)-(III), or pharmaceutically acceptable salt or solvate thereof, wherein R¹¹ is optionally substituted C1-C4 fluoroalkyl. Another embodiment provides the compound of Formula (I)-(III), or pharmaceutically acceptable salt or solvate thereof, wherein R¹¹ is —CH₂CF₃. Another embodiment provides the compound of Formula (I)-(III), or pharmaceutically acceptable salt or solvate thereof, wherein R¹¹ is optionally substituted C3-C6 cycloalkyl.

Another embodiment provides the compound of Formula (I)-(III), or pharmaceutically acceptable salt or solvate thereof, wherein Z is

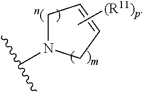

Another embodiment provides the compound of Formula (I)-(III), or pharmaceutically acceptable salt or solvate thereof, wherein m is 0. Another embodiment provides the compound of Formula (I)-(III), or pharmaceutically acceptable salt or solvate thereof, wherein m is 1. Another embodiment provides the compound of Formula (I)-(III), or pharmaceutically acceptable salt or solvate thereof, wherein m is 2. Another embodiment provides the compound of Formula (I)-(III), or pharmaceutically acceptable salt or solvate thereof, wherein n is 1. Another embodiment provides the compound of Formula (I)-(III), or pharmaceutically acceptable salt or solvate thereof, wherein n is 2. Another embodiment provides the compound of Formula (I)-(III), or pharmaceutically acceptable salt or solvate thereof, wherein p is 1. Another embodiment provides the compound of Formula (I)-(III), or pharmaceutically acceptable salt or solvate thereof, wherein p is 2.

Another embodiment provides the compound of Formula (I)-(III), or pharmaceutically acceptable salt or solvate thereof, wherein R^{II} is optionally substituted C1-C6 alkyl. Another embodiment provides the compound of Formula (I)-(III), or pharmaceutically acceptable salt or solvate thereof, wherein R¹¹ is optionally substituted C1-C4 alkyl. Another embodiment provides the compound of Formula (I)-(III), or pharmaceutically acceptable salt or solvate thereof, wherein R¹¹ is optionally substituted C1-C3 alkyl. Another embodiment provides the compound of Formula (I)-(III), or pharmaceutically acceptable salt or solvate thereof, wherein R¹¹ is optionally substituted C1-C2 alkyl. Another embodiment provides the compound of Formula (I)-(III), or pharmaceutically acceptable salt or solvate thereof, wherein R¹¹ is optionally substituted C1-C4 fluoroalkyl. Another embodiment provides the compound of Formula (I)-(III), or pharmaceutically acceptable salt or solvate thereof, wherein R¹¹ is —CH₂CF₃. Another embodiment provides the compound of Formula (I)-(III), or pharmaceutically acceptable salt or solvate thereof, wherein R¹¹ is optionally substituted C3-C6 cycloalkyl.

Another embodiment provides the compound of Formula (I)-(III), or pharmaceutically acceptable salt or solvate thereof, wherein W is NH and ring A is an optionally substituted morpholine.

Another embodiment provides the compound of Formula (I)-(III), or pharmaceutically acceptable salt or solvate thereof, wherein W is NH and Z is 3-(2,2,2-trifluoroethyl)pyrrolidine.

Another embodiment provides the compound of Formula (I)-(III), or pharmaceutically acceptable salt or solvate thereof, wherein ring A is an optionally substituted morpholine and Z is 3-(2,2,2-trifluoroethyl)pyrrolidine.

Another embodiment provides the compound of Formula (I)-(III), or pharmaceutically acceptable salt or solvate thereof, wherein W is NH, ring A is an optionally substituted morpholine, and Z is 3-(2,2,2-trifluoroethyl)pyrrolidine.

Another embodiment provides the compound of Formula (I)-(III), or pharmaceutically acceptable salt or solvate thereof, wherein R is C1-C4 optionally substituted alkyl, W is NH and ring A is an optionally substituted morpholine.

Another embodiment provides the compound of Formula (I)-(III), or pharmaceutically acceptable salt or solvate thereof, wherein R is C1-C4 optionally substituted alkyl, W is NH and Z is 3-(2,2,2-trifluoroethyl)pyrrolidine.

Another embodiment provides the compound of Formula (I)-(III), or pharmaceutically acceptable salt or solvate thereof, wherein R is C1-C4 optionally substituted alkyl, ring A is an optionally substituted morpholine and Z is 3-(2,2,2-trifluoroethyl)pyrrolidine.

Another embodiment provides the compound of Formula (I)-(III), or pharmaceutically acceptable salt or solvate thereof, wherein R is C1-C4 optionally substituted alkyl, W is NH, ring A is an optionally substituted morpholine, and Z is 3-(2,2,2-trifluoroethyl)pyrrolidine.

Another embodiment provides the compound of Formula (I)-(III), or pharmaceutically acceptable salt or solvate thereof, wherein X is C—H, Y is C—H, W is NH and ring A is an optionally substituted morpholine.

Another embodiment provides the compound of Formula (I)-(III), or pharmaceutically acceptable salt or solvate thereof, wherein X is C—H, Y is C—H, W is NH and Z is 3-(2,2,2-trifluoroethyl)pyrrolidine.

Another embodiment provides the compound of Formula (I)-(III), or pharmaceutically acceptable salt or solvate thereof, wherein X is C—H, Y is C—H, ring A is an optionally substituted morpholine and Z is 3-(2,2,2-trifluoroethyl)pyrrolidine.

Another embodiment provides the compound of Formula (I)-(III), or pharmaceutically acceptable salt or solvate thereof, wherein X is C—H, Y is C—H, W is NH, ring A is an optionally substituted morpholine, and Z is 3-(2,2,2-trifluoroethyl)pyrrolidine.

Another embodiment provides the compound of Formula (I)-(III), or pharmaceutically acceptable salt or solvate thereof, wherein X is C—H, Y is C—H, R is C1-C4 optionally substituted alkyl, W is NH and ring A is an optionally substituted morpholine.

Another embodiment provides the compound of Formula (I)-(III), or pharmaceutically acceptable salt or solvate thereof, wherein X is C—H, Y is C—H, R is C1-C4 optionally substituted alkyl, W is NH and Z is 3-(2,2,2-trifluoroethyl)pyrrolidine.

Another embodiment provides the compound of Formula (I)-(III), or pharmaceutically acceptable salt or solvate thereof, wherein X is C—H, Y is C—H, R is C1-C4 optionally substituted alkyl, ring A is an optionally substituted morpholine and Z is 3-(2,2,2-trifluoroethyl)pyrrolidine.

Another embodiment provides the compound of Formula (I)-(III), or pharmaceutically acceptable salt or solvate thereof, wherein X is C—H, Y is C—H, R is C1-C4 optionally substituted alkyl, W is NH, ring A is an optionally substituted morpholine, and Z is 3-(2,2,2-trifluoroethyl)pyrrolidine.

One embodiment provides a compound, or pharmaceutically acceptable salt or solvate thereof, having the structure of Formula (IV):

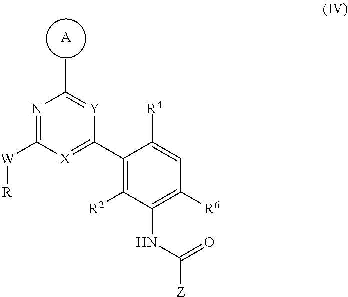

wherein,
ring A is a 5- or 6-membered optionally substituted heteroaryl, or 5- or 6-membered optionally substituted heterocyclyl;
W is NH, or NR⁷;
X is N, C—H, C—F, or C—CN;
Y is N, C—F, or C—H;
R is —SO₂—(C1-C8 optionally substituted alkyl), —SO₂—(C3-C6 optionally substituted cycloalkyl), —SO₂—(C4-C8 optionally substituted cycloalkylalkyl), —SO₂—(C3-C6 optionally substituted heterocyclyl), —SO₂—(C3-C6 optionally substituted heterocyclylalkyl), —SO₂—N(R⁸)R⁹—C1-C8 optionally substituted alkyl, —CO₂—C1-C8 optionally substituted alkyl, or —CO—N(R⁸)R⁹—C1-C8 optionally substituted alkyl;
R² is H, D or F;
R⁴ is halogen, optionally substituted C1-C3 alkyl, -CD₃, or optionally substituted C1-C3 alkoxy;
R⁶ is H, D, Cl or F;
R⁷ is C1-C8 optionally substituted alkyl; or R and R⁷ optionally join to form an optionally substituted heterocyclyl ring;
each R⁸ and R⁹ is independently selected from H or C1-C8 optionally substituted alkyl;
Z is (a)

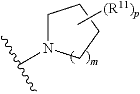

wherein m is 0, 1, 2, or 3; p is 0, 1, 2, 3, or 4; and
each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —S-alkyl, optionally substituted —SO₂alkyl, optionally substituted C3-C6 cycloalkylalkyl, optionally substituted heterocyclyl, or optionally substituted heterocyclylalkyl; or two R¹¹ groups together form an oxo; or
(b)

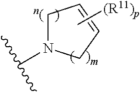

wherein m is 0, 1, 2, or 3; n is 0, 1, 2, or 3; p is 0, 1, 2, 3, or 4; and each R¹¹ is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —S-alkyl, optionally substituted —SO₂alkyl, optionally substituted C3-C6 cycloalkylalkyl, optionally substituted heterocyclyl, or optionally substituted heterocyclylalkyl; or two R¹¹ groups together form an oxo.

Another embodiment provides the compound of Formula (IV), or pharmaceutically acceptable salt or solvate thereof, wherein ring A is a 5- or 6-membered optionally substituted heteroaryl.

Another embodiment provides the compound of Formula (IV), or pharmaceutically acceptable salt or solvate thereof, wherein ring A is an optionally substituted pyridine or optionally substituted pyrrazole. Another embodiment provides the compound of Formula (IV), or pharmaceutically acceptable salt or solvate thereof, wherein ring A is an optionally substituted 4-pyridinyl or optionally substituted 3-pyrrazole.

Another embodiment provides the compound of Formula (IV), or pharmaceutically acceptable salt or solvate thereof, wherein ring A is a 5- or 6-membered optionally substituted heterocyclyl.

Another embodiment provides the compound of Formula (IV), or pharmaceutically acceptable salt or solvate thereof, wherein ring A is an optionally substituted dihydropyran, optionally substituted tetrahydropyran, optionally substituted dihydrofuran, or optionally substituted tetrahydrofuran. Another embodiment provides the compound of Formula (IV), or pharmaceutically acceptable salt or solvate thereof, wherein ring A is an optionally substituted dihydro-2H-pyran-4-yl, optionally substituted tetrahydropyran-4-yl, optionally substituted 2,5-dihydrofuran-3-yl, optionally substituted tetrahydrofuran-3-yl, 3-oxabicyclo[3.1.0]hexan-1-yl, or 3-oxabicyclo[4.1.0]heptan-6-yl.

Another embodiment provides the compound of Formula (IV), or pharmaceutically acceptable salt or solvate thereof, wherein ring A is an optionally substituted morpholine.

Another embodiment provides the compound of Formula (IV), or pharmaceutically acceptable salt or solvate thereof, wherein W is NH.

Another embodiment provides the compound of Formula (IV), or pharmaceutically acceptable salt or solvate thereof, wherein NR⁷. Another embodiment provides the compound of Formula (IV), or pharmaceutically acceptable salt or solvate thereof, wherein R⁷ is C1-C8 optionally substituted alkyl. Another embodiment provides the compound of Formula (IV), or pharmaceutically acceptable salt or solvate thereof, wherein R and R⁷ optionally join to form an optionally substituted heterocyclyl ring.

Another embodiment provides the compound of Formula (IV), or pharmaceutically acceptable salt or solvate thereof, wherein R is —SO₂—(C1-C8 optionally substituted alkyl).

Another embodiment provides the compound of Formula (IV), or pharmaceutically acceptable salt or solvate thereof, wherein R is —SO₂—(C3-C6 optionally substituted cycloalkyl).

Another embodiment provides the compound of Formula (IV), or pharmaceutically acceptable salt or solvate thereof, wherein R is —SO₂—(C4-C8 optionally substituted cycloalkylalkyl).

Another embodiment provides the compound of Formula (IV), or pharmaceutically acceptable salt or solvate thereof, wherein R is —SO₂—(C3-C6 optionally substituted heterocyclyl).

Another embodiment provides the compound of Formula (IV), or pharmaceutically acceptable salt or solvate thereof, wherein R is —SO₂—(C3-C6 optionally substituted heterocyclylalkyl).

Another embodiment provides the compound of Formula (IV), or pharmaceutically acceptable salt or solvate thereof, wherein R is —SO₂—N(R′)R⁹—C1-C8 optionally substituted alkyl. Another embodiment provides the compound of Formula (IV), or pharmaceutically acceptable salt or solvate thereof, wherein each R⁸ and R⁹ is independently selected from H or C1-C8 optionally substituted alkyl.

Another embodiment provides the compound of Formula (IV), or pharmaceutically acceptable salt or solvate thereof, wherein R is —CO₂—C1-C8 optionally substituted alkyl.

Another embodiment provides the compound of Formula (IV), or pharmaceutically acceptable salt or solvate thereof, wherein R is —CO—N(R⁸)R⁹—C1-C8 optionally substituted alkyl. Another embodiment provides the compound of Formula (IV), or pharmaceutically acceptable salt or solvate thereof, wherein each R⁸ and R⁹ is independently selected from H or C1-C8 optionally substituted alkyl.

Another embodiment provides the compound of Formula (IV), or pharmaceutically acceptable salt or solvate thereof, wherein Z is

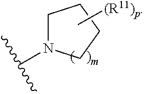

Another embodiment provides the compound of Formula (IV), or pharmaceutically acceptable salt or solvate thereof, wherein m is 0. Another embodiment provides the compound of Formula (IV), or pharmaceutically acceptable salt or solvate thereof, wherein m is 1. Another embodiment provides the compound of Formula (IV), or pharmaceutically acceptable salt or solvate thereof, wherein m is 2. Another embodiment provides the compound of Formula (IV), or pharmaceutically acceptable salt or solvate thereof, wherein p is 1. Another embodiment provides the compound of Formula (IV), or pharmaceutically acceptable salt or solvate thereof, wherein p is 2.

Another embodiment provides the compound of Formula (IV), or pharmaceutically acceptable salt or solvate thereof, wherein Z is

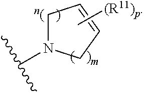

Another embodiment provides the compound of Formula (IV), or pharmaceutically acceptable salt or solvate thereof, wherein m is 0. Another embodiment provides the compound of Formula (IV), or pharmaceutically acceptable salt or solvate thereof, wherein m is 1. Another embodiment provides the compound of Formula (IV), or pharmaceutically acceptable salt or solvate thereof, wherein m is 2. Another embodiment provides the compound of Formula (IV), or pharmaceutically acceptable salt or solvate thereof, wherein n is 1. Another embodiment provides the compound of Formula (IV), or pharmaceutically acceptable salt or solvate thereof, wherein n is 2. Another embodiment provides the compound of Formula (IV), or pharmaceutically acceptable salt or solvate thereof, wherein p is 1. Another embodiment provides the compound of Formula (IV), or pharmaceutically acceptable salt or solvate thereof, wherein p is 2.

Another embodiment provides the compound of Formula (IV), or pharmaceutically acceptable salt or solvate thereof, wherein R¹¹ is optionally substituted C1-C6 alkyl. Another embodiment provides the compound of Formula (IV), or pharmaceutically acceptable salt or solvate thereof, wherein R¹¹ is optionally substituted C1-C4 alkyl. Another embodiment provides the compound of Formula (IV), or pharmaceutically acceptable salt or solvate thereof, wherein R¹¹ is optionally substituted C1-C3 alkyl. Another embodiment provides the compound of Formula (IV), or pharmaceutically acceptable salt or solvate thereof, wherein R¹¹ is optionally substituted C1-C2 alkyl. Another embodiment provides the compound of Formula (IV), or pharmaceutically acceptable salt or solvate thereof, wherein R¹¹ is optionally substituted C1-C4 fluoroalkyl.

Another embodiment provides the compound of Formula (IV), or pharmaceutically acceptable salt or solvate thereof, wherein R¹¹ is —CH₂CF₃. Another embodiment provides the compound of Formula (IV), or pharmaceutically acceptable salt or solvate thereof, wherein R¹¹ is optionally substituted C3-C6 cycloalkyl.

Another embodiment provides the compound of Formula (IV), or pharmaceutically acceptable salt or solvate thereof, wherein W is NH and ring A is an optionally substituted morpholine.

Another embodiment provides the compound of Formula (IV), or pharmaceutically acceptable salt or solvate thereof, wherein W is NH and Z is 3-(2,2,2-trifluoroethyl)pyrrolidine.

Another embodiment provides the compound of Formula (IV), or pharmaceutically acceptable salt or solvate thereof, wherein ring A is an optionally substituted morpholine and Z is 3-(2,2,2-trifluoroethyl)pyrrolidine.

Another embodiment provides the compound of Formula (IV), or pharmaceutically acceptable salt or solvate thereof, wherein W is NH, ring A is an optionally substituted morpholine, and Z is 3-(2,2,2-trifluoroethyl)pyrrolidine.

Another embodiment provides the compound of Formula (IV), or pharmaceutically acceptable salt or solvate thereof, wherein X is C—H, Y is C—H, W is NH and ring A is an optionally substituted morpholine.

Another embodiment provides the compound of Formula (IV), or pharmaceutically acceptable salt or solvate thereof, wherein X is C—H, Y is C—H, W is NH and Z is 3-(2,2,2-trifluoroethyl)pyrrolidine.

Another embodiment provides the compound of Formula (IV), or pharmaceutically acceptable salt or solvate thereof, wherein X is C—H, Y is C—H, ring A is an optionally substituted morpholine and Z is 3-(2,2,2-trifluoroethyl)pyrrolidine.

Another embodiment provides the compound of Formula (IV), or pharmaceutically acceptable salt or solvate thereof, wherein X is C—H, Y is C—H, W is NH, ring A is an optionally substituted morpholine, and Z is 3-(2,2,2-trifluoroethyl)pyrrolidine.

Another embodiment provides the compound of Formula (I)-(IV), or pharmaceutically acceptable salt or solvate thereof, wherein X is N; and Y is C—H.

Another embodiment provides the compound of Formula (I)-(IV), or pharmaceutically acceptable salt or solvate thereof, wherein X is N; and Y is N.

Another embodiment provides the compound of Formula (I)-(IV), or pharmaceutically acceptable salt or solvate thereof, wherein X is C—H; and Y is C—H.

Another embodiment provides the compound of Formula (I)-(IV), or pharmaceutically acceptable salt or solvate thereof, wherein X is C—H; and Y is N.

Another embodiment provides the compound of Formula (I)-(IV), or pharmaceutically acceptable salt or solvate thereof, wherein X is C—F; and Y is C—H.

Another embodiment provides the compound of Formula (I)-(IV), or pharmaceutically acceptable salt or solvate thereof, wherein X is C—H; and Y is C—F.

Another embodiment provides the compound of Formula (I)-(IV), or pharmaceutically acceptable salt or solvate thereof, wherein R² is H.

Another embodiment provides the compound of Formula (I)-(IV), or pharmaceutically acceptable salt or solvate thereof, wherein R⁴ is optionally substituted C1-C3 alkyl.

Another embodiment provides the compound of Formula (I)-(IV), or pharmaceutically acceptable salt or solvate thereof, wherein R⁴ is optionally substituted C1 alkyl.

Another embodiment provides the compound of Formula (I)-(IV), or pharmaceutically acceptable salt or solvate thereof, wherein R⁴ is CH₃.

Another embodiment provides the compound of Formula (I)-(IV), or pharmaceutically acceptable salt or solvate thereof, wherein R⁶ is H.

In some embodiments, the heteroaromatic RAF kinase inhibitory compound as described herein has a structure provided in Table 1.

TABLE 1

| Synthetic | | |
|---|---|---|
| Chemistry | | |
| Example | Compound Structure | Compound Name |
| 1 | 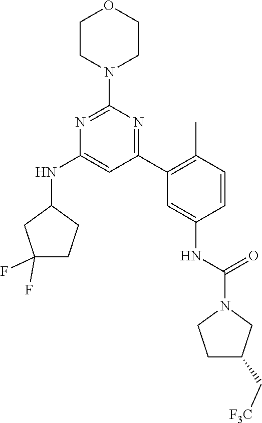 [SMILES: Cc1ccc(NC(=O)N2CC[C@@H](CC(F)(F)F)C2)cc1-c1cc(NC2CCC(F)(F)C2)nc(N2CCOCC2)n1] | (3S)-N-(3-(6-((3,3- difluorocyclopentyl)amino)-2- morpholinopyrimidin-4-yl)-4- methylphenyl)-3-(2,2,2- trifluoroethyl)pyrrolidine-1-carboxamide |
| 2 | 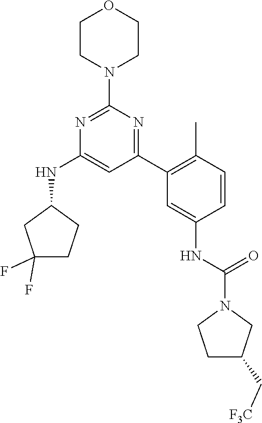 [SMILES: Cc1ccc(NC(=O)N2CC[C@@H](CC(F)(F)F)C2)cc1-c1cc(N[C@@H]2CCC(F)(F)C2)nc(N2CCOCC2)n1] | (S)-N-(3-(6-(((R)-3,3- difluorocyclopentyl)amino)-2- morpholinopyrimidin-4-yl)-4- methylphenyl)-3-(2,2,2- trifluoroethyl)pyrrolidine-1-carboxamide |
| 3 | 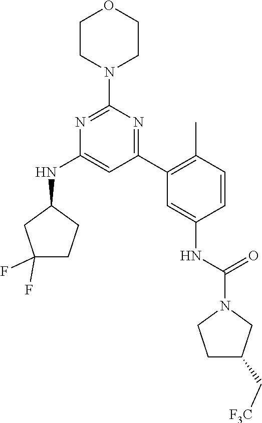 [SMILES: Cc1ccc(NC(=O)N2CC[C@@H](CC(F)(F)F)C2)cc1-c1cc(N[C@H]2CCC(F)(F)C2)nc(N2CCOCC2)n1] | (S)-N-(3-(6-(((S)-3,3- difluorocyclopentyl)amino)-2- morpholinopyrimidin-4-yl)-4- methylphenyl)-3-(2,2,2- trifluoroethyl)pyrrolidine-1-carboxamide |
| 4 and 5 | 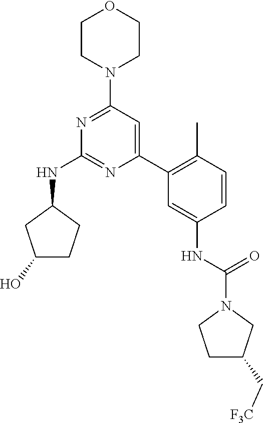 [SMILES: Cc1ccc(NC(=O)N2CC[C@@H](CC(F)(F)F)C2)cc1-c1cc(N2CCOCC2)nc(N[C@H]2CC[C@H](O)C2)n1] | (S)-N-(3-(2-(((15,3S)-3- hydroxycyclopentyl)amino)-6- morpholinopyrimidin-4-yl)-4- methylphenyl)-3-(2,2,2- trifluoroethyl)pyrrolidine-1-carboxamide and (S)-N-(3-(2-(((1R,3R)-3- hydroxycyclopentyl)amino)-6- morpholinopyrimidin-4-yl)-4- methylphenyl)-3-(2,2,2- trifluoroethyl)pyrrolidine-1-carboxamide |
| | + | |
| | 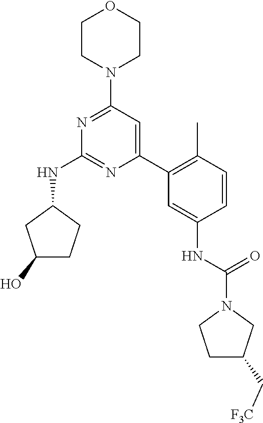 [SMILES: Cc1ccc(NC(=O)N2CC[C@@H](CC(F)(F)F)C2)cc1-c1cc(N2CCOCC2)nc(N[C@@H]2CC[C@@H](O)C2)n1] | |
| 6 | 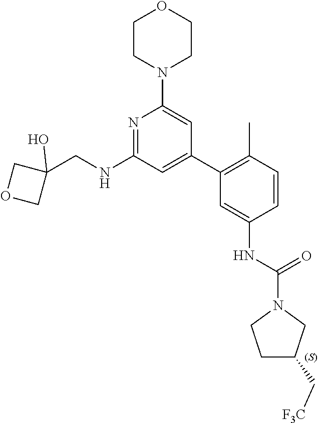 | (S)-N-(3-(2-(((3-hydroxyoxetan-3- yl)methyl)amino)-6-morpholinopyridin- 4-yl)-4-methylphenyl)-3-(2,2,2- trifluoroethyl)pyrrolidine-1-carboxamide |
| 7 | 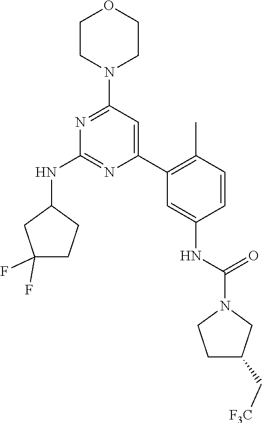 [SMILES: Cc1ccc(NC(=O)N2CC[C@@H](CC(F)(F)F)C2)cc1-c1cc(N2CCOCC2)nc(NC2CCC(F)(F)C2)n1] | (3S)-N-(3-(2-((3,3- difluorocyclopentyl)amino)-6- morpholinopyrimidin-4-yl)-4- methylphenyl)-3-(2,2,2- trifluoroethyl)pyrrolidine-1-carboxamide |
| 8 and 9 | 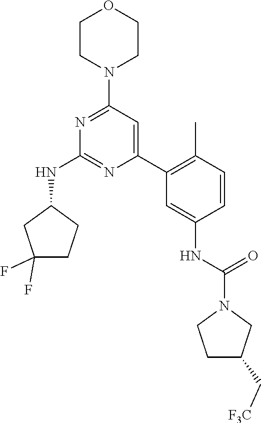 [SMILES: Cc1ccc(NC(=O)N2CC[C@@H](CC(F)(F)F)C2)cc1-c1cc(N2CCOCC2)nc(N[C@@H]2CCC(F)(F)C2)n1] | (S)-N-(3-(2-(((R)-3,3- difluorocyclopentyl)amino)-6- morpholinopyrimidin-4-yl)-4- methylphenyl)-3-(2,2,2- trifluoroethyl)pyrrolidine-1-carboxamide and (S)-N-(3-(2-(((S)-3,3- difluorocyclopentyl)amino)-6- morpholinopyrimidin-4-yl)-4- methylphenyl)-3-(2,2,2- trifluoroethyl)pyrrolidine-1-carboxamide |
| | + | |
| | 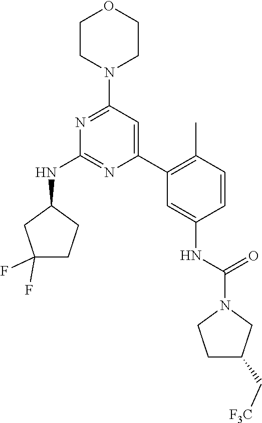 [SMILES: Cc1ccc(NC(=O)N2CC[C@@H](CC(F)(F)F)C2)cc1-c1cc(N2CCOCC2)nc(N[C@H]2CCC(F)(F)C2)n1] | |
| 10 | 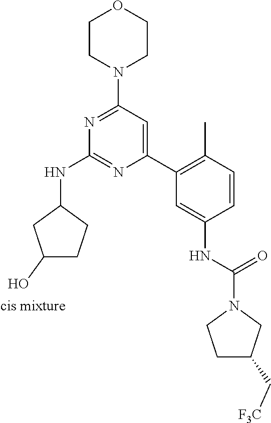 [SMILES: Cc1ccc(NC(=O)N2CC[C@@H](CC(F)(F)F)C2)cc1-c1cc(N2CCOCC2)nc(NC2CCC(O)C2)n1] | (3S)-N-[3-(2-[(cis-3- hydroxycyclopentyl]amino]-6- (morpholin-4-yl)pyrimidin-4-yl)-4- methylphenyl]-3-(2,2,2- trifluoroethyl)pyrrolidine-1-carboxamide |
| 11 and 12 | 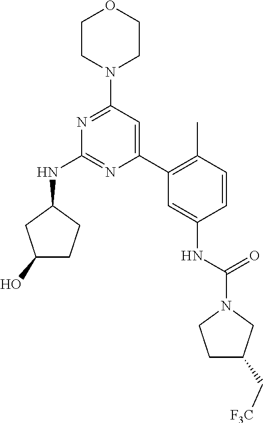 [SMILES: Cc1ccc(NC(=O)N2CC[C@@H](CC(F)(F)F)C2)cc1-c1cc(N2CCOCC2)nc(N[C@H]2CC[C@@H](O)C2)n1] | (S)-N-(3-(2-(((1S,3R)-3- hydroxycyclopentyl)amino)-6- morpholinopyrimidin-4-yl)-4- methylphenyl)-3-(2,2,2- trifluoroethyl)pyrrolidine-1-carboxamide and (S)-N-(3-(2-(((1R,3S)-3- hydroxycyclopentyl)amino)-6- morpholinopyrimidin-4-yl)-4- methylphenyl)-3-(2,2,2- trifluoroethyl)pyrrolidine-1-carboxamide |
| | + | |
| | 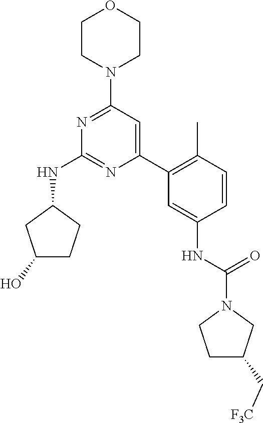 [SMILES: Cc1ccc(NC(=O)N2CC[C@@H](CC(F)(F)F)C2)cc1-c1cc(N2CCOCC2)nc(N[C@@H]2CC[C@H](O)C2)n1] | |
| 13 | 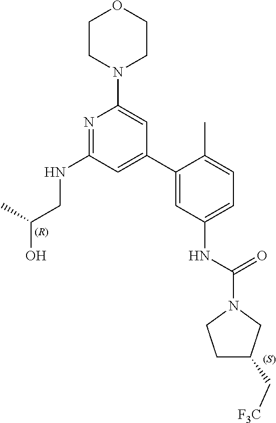 | (S)-N-(3-(2-(((R)-2- hydroxypropyl)amino)-6- morpholinopyridin-4-yl)-4- methylphenyl)-3-(2,2,2- trifluoroethyl)pyrrolidine-1-carboxamide |
| 14 | 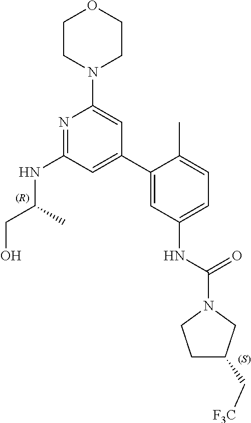 | (S)-N-(3-(2-(((R)-1-hydroxypropan-2- yl)amino)-6-morpholinopyridin-4-yl)-4- methylphenyl)-3-(2,2,2- trifluoroethyl)pyrrolidine-1-carboxamide |
| 15 | 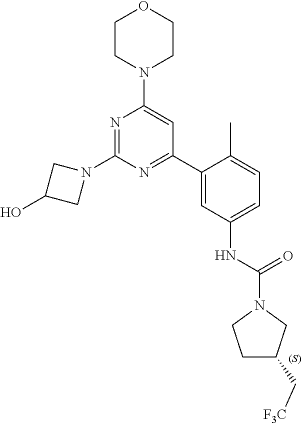 | (S)-N-(3-(2-(3-hydroxyazetidin-1-yl)-6- morpholinopyrimidin-4-yl)-4- methylphenyl)-3-(2,2,2- trifluoroethyl)pyrrolidine-1-carboxamide |
| 16 | 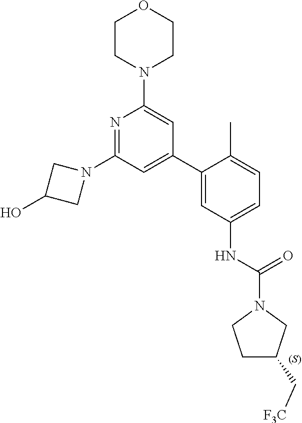 | (S)-N-(3-(2-(3-hydroxyazetidin-1-yl)-6- morpholinopyridin-4-yl)-4- methylphenyl)-3-(2,2,2- trifluoroethyl)pyrrolidine-1-carboxamide |
| 17 | 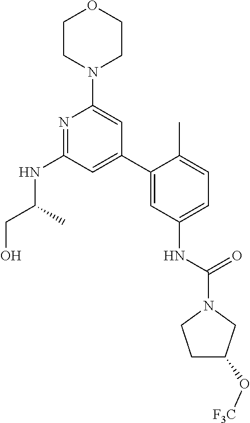 [SMILES: Cc1ccc(NC(=O)N2CC[C@@H](OC(F)(F)F)C2)cc1-c1cc(N[C@H](C)CO)nc(N2CCOCC2)c1] | (R)-N-(3-(2-(((R)-1-hydroxypropan-2- yl)amino)-6-morpholinopyridin-4-yl)-4- methylphenyl)-3- (trifluoromethoxy)pyrrolidine-1- carboxamide |
| 18 | 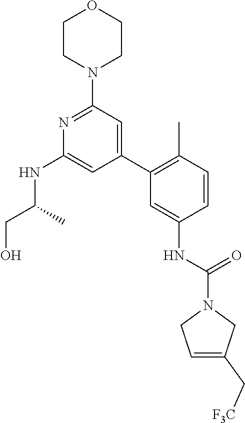 [SMILES: Cc1ccc(NC(=O)N2CC=C(CC(F)(F)F)C2)cc1-c1cc(N[C@H](C)CO)nc(N2CCOCC2)c1] | (R)-N-(3-(2-((1-hydroxypropan-2- yl)amino)-6-morpholinopyridin-4-yl)-4- methylphenyl)-3-(2,2,2-trifluoroethyl)- 2,5-dihydro-1H-pyrrole-1-carboxamide |
| 19 | 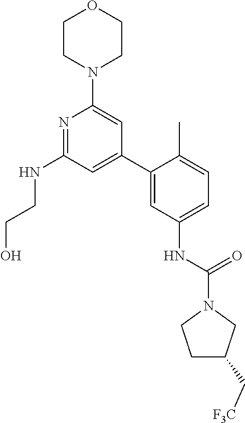 [SMILES: Cc1ccc(NC(=O)N2CC[C@@H](CC(F)(F)F)C2)cc1-c1cc(NCCO)nc(N2CCOCC2)c1] | (S)-N-(3-(2-((2-hydroxyethyl)amino)-6- morpholinopyridin-4-yl)-4- methylphenyl)-3-(2,2,2- trifluoroethyl)pyrrolidine-1-carboxamide |
| 20 | 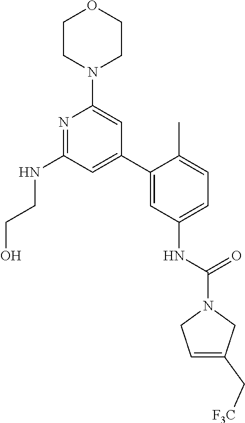 [SMILES: Cc1ccc(NC(=O)N2CC=C(CC(F)(F)F)C2)cc1-c1cc(NCCO)nc(N2CCOCC2)c1] | N-(3-(2-((2-hydroxyethyl)amino)-6- morpholinopyridin-4-yl)-4- methylphenyl)-3-(2,2,2-trifluoroethyl)- 2,5-dihydro-1H-pyrrole-1-carboxamide |
| 21 | 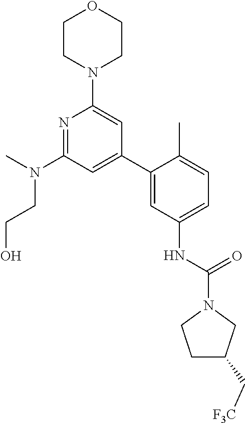 [SMILES: Cc1ccc(NC(=O)N2CC[C@@H](CC(F)(F)F)C2)cc1-c1cc(N(C)CCO)nc(N2CCOCC2)c1] | (S)-N-(3-(2-((2- hydroxyethyl)(methyl)amino)-6- morpholinopyridin-4-yl)-4- methylphenyl)-3-(2,2,2- trifluoroethyl)pyrrolidine-1-carboxamide |
| 22 | 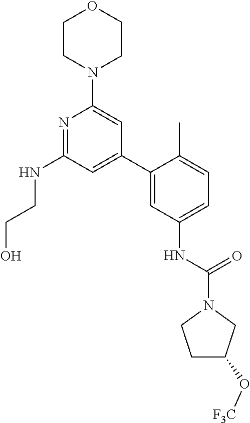 [SMILES: Cc1ccc(NC(=O)N2CC[C@@H](OC(F)(F)F)C2)cc1-c1cc(NCCO)nc(N2CCOCC2)c1] | (R)-N-(3-(2-((2-hydroxyethyl)amino)-6- morpholinopyridin-4-yl)-4- methylphenyl)-3- (trifluoromethoxy)pyrrolidine-1- carboxamide |
| 23 | 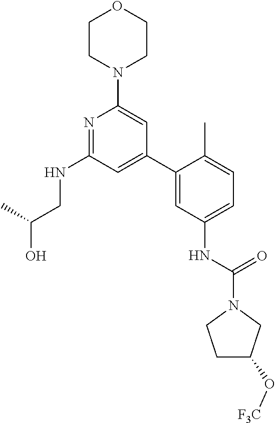 [SMILES: Cc1ccc(NC(=O)N2CC[C@@H](OC(F)(F)F)C2)cc1-c1cc(NC[C@@H](C)O)nc(N2CCOCC2)c1] | (R)-N-(3-(2-(((R)-2- hydroxypropyl)amino)-6- morpholinopyridin-4-yl)-4- methylphenyl)-3- (trifluoromethoxy)pyrrolidine-1- carboxamide |
| 24 | 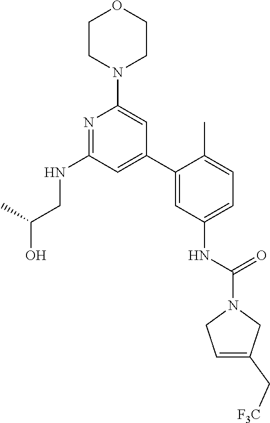 [SMILES: Cc1ccc(NC(=O)N2CC=C(CC(F)(F)F)C2)cc1-c1cc(NC[C@@H](C)O)nc(N2CCOCC2)c1] | (R)-N-(3-(2-((2-hydroxypropyl)amino)-6- morpholinopyridin-4-yl)-4- methylphenyl)-3-(2,2,2-trifluoroethyl)- 2,5-dihydro-1H-pyrrole-1-carboxamide |
| 25 | 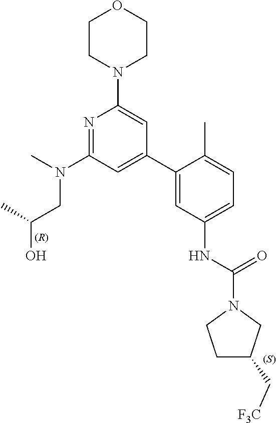 | (S)-N-(3-(2-(((R)-2- hydroxypropyl)(methyl)amino)-6- morpholinopyridin-4-yl)-4- methylphenyl)-3-(2,2,2- trifluoroethyl)pyrrolidine-1-carboxamide |
| 26 | 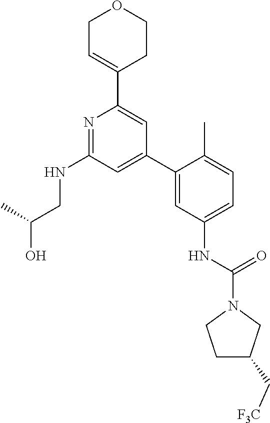 [SMILES: Cc1ccc(NC(=O)N2CC[C@@H](CC(F)(F)F)C2)cc1-c1cc(NC[C@@H](C)O)nc(C2=CCOCC2)c1] | (S)-N-(3-(2-(3,6-dihydro-2H-pyran-4-yl)- 6-(((R)-2-hydroxypropyl)amino)pyridin- 4-yl)-4-methylphenyl)-3-(2,2,2- trifluoroethyl)pyrrolidine-1-carboxamide |
| 27 | 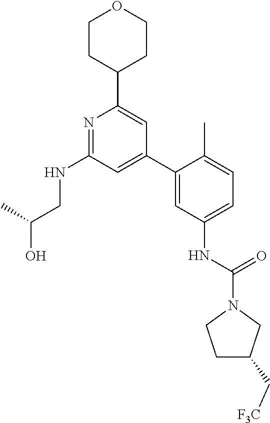 [SMILES: Cc1ccc(NC(=O)N2CC[C@@H](CC(F)(F)F)C2)cc1-c1cc(NC[C@@H](C)O)nc(C2CCOCC2)c1] | (S)-N-(3-(2-(((R)-2- hydroxypropyl)amino)-6-(tetrahydro-2H- pyran-4-yl)pyridin-4-yl)-4- methylphenyl)-3-(2,2,2- trifluoroethyl)pyrrolidine-1-carboxamide |
| 28 | 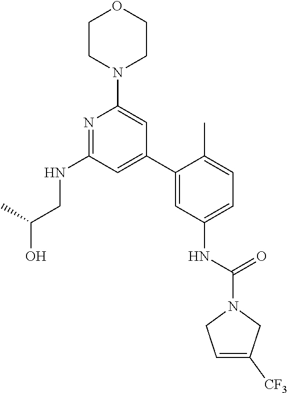 [SMILES: Cc1ccc(NC(=O)N2CC=C(C(F)(F)F)C2)cc1-c1cc(NC[C@@H](C)O)nc(N2CCOCC2)c1] | (R)-N-(3-(2-((2-hydroxypropyl)amino)-6- morpholinopyridin-4-yl)-4- methylphenyl)-3-(trifluoromethyl)-2,5- dihydro-1H-pyrrole-1-carboxamide |
| 29 | 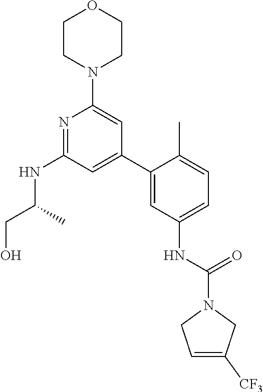 [SMILES: Cc1ccc(NC(=O)N2CC=C(C(F)(F)F)C2)cc1-c1cc(N[C@H](C)CO)nc(N2CCOCC2)c1] | (R)-N-(3-(2-((1-hydroxypropan-2- yl)amino)-6-morpholinopyridin-4-yl)-4- methylphenyl)-3-(trifluoromethyl)-2,5- dihydro-1H-pyrrole-1-carboxamide |
| 30 | 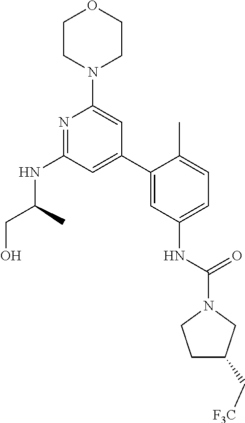 [SMILES: Cc1ccc(NC(=O)N2CC[C@@H](CC(F)(F)F)C2)cc1-c1cc(N[C@@H](C)CO)nc(N2CCOCC2)c1] | (S)-N-(3-(2-(((S)-1-hydroxypropan-2- yl)amino)-6-morpholinopyridin-4-yl)-4- methylphenyl)-3-(2,2,2- trifluoroethyl)pyrrolidine-1-carboxamide |
| 31 | 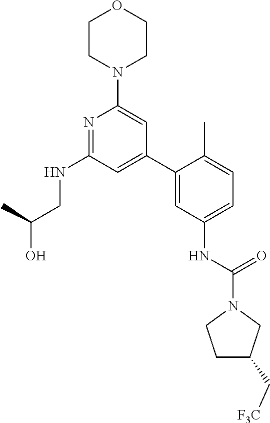 [SMILES: Cc1ccc(NC(=O)N2CC[C@@H](CC(F)(F)F)C2)cc1-c1cc(NC[C@H](C)O)nc(N2CCOCC2)c1] | (S)-N-(3-(2-(((S)-2- hydroxypropyl)amino)-6- morpholinopyridin-4-yl)-4- methylphenyl)-3-(2,2,2- trifluoroethyl)pyrrolidine-1-carboxamide |
| 32 | 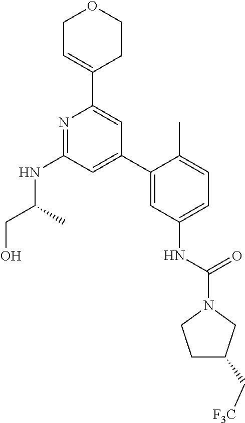 [SMILES: Cc1ccc(NC(=O)N2CC[C@@H](CC(F)(F)F)C2)cc1-c1cc(N[C@H](C)CO)nc(C2=CCOCC2)c1] | (S)-N-(3-(2-(3,6-dihydro-2H-pyran-4-yl)- 6-(((R)-1-hydroxypropan-2- yl)amino)pyridin-4-yl)-4-methylphenyl)- 3-(2,2,2-trifluoroethyl)pyrrolidine-1- carboxamide |
| 33 | 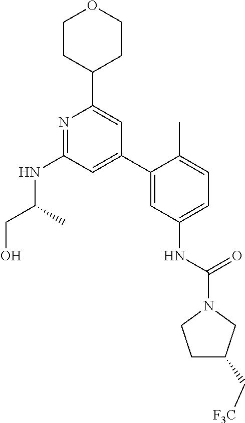 [SMILES: Cc1ccc(NC(=O)N2CC[C@@H](CC(F)(F)F)C2)cc1-c1cc(N[C@H](C)CO)nc(C2CCOCC2)c1] | (S)-N-(3-(2-(((R)-1-hydroxypropan-2- yl)amino)-6-(tetrahydro-2H-pyran-4- yl)pyridin-4-yl)-4-methylphenyl)-3- (2,2,2-trifluoroethyl)pyrrolidine-1- carboxamide |
| 34 | 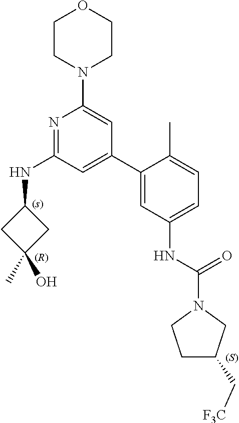 | (S)-N-(3-(2-(((1S,3R)-3-hydroxy-3- methylcyclobutyl)amino)-6- morpholinopyridin-4-yl)-4- methylphenyl)-3-(2,2,2- trifluoroethyl)pyrrolidine-1-carboxamide |
| 35 | 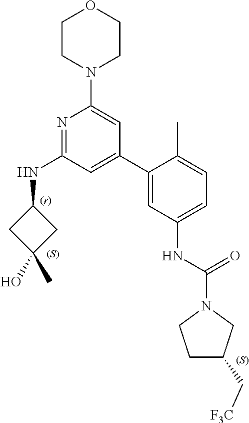 | (S)-N-(3-(2-(((1R,3S)-3-hydroxy-3- methylcyclobutyl)amino)-6- morpholinopyridin-4-yl)-4- methylphenyl)-3-(2,2,2- trifluoroethyl)pyrrolidine-1-carboxamide |
| 36 | 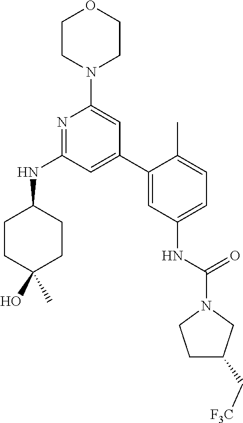 [SMILES: Cc1ccc(NC(=O)N2CC[C@@H](CC(F)(F)F)C2)cc1-c1cc(N[C@H]2CC[C@@](C)(O)CC2)nc(N2CCOCC2)c1] | (S)-N-(3-(2-(((1S,4R)-4-hydroxy-4- methylcyclohexyl)amino)-6- morpholinopyridin-4-yl)-4- methylphenyl)-3-(2,2,2- trifluoroethyl)pyrrolidine-1-carboxamide |
| 37 | 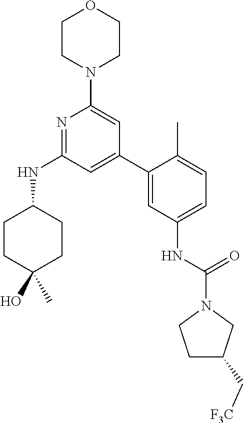 [SMILES: Cc1ccc(NC(=O)N2CC[C@@H](CC(F)(F)F)C2)cc1-c1cc(N[C@H]2CC[C@](C)(O)CC2)nc(N2CCOCC2)c1] | (S)-N-(3-(2-(((1R,4S)-4-hydroxy-4- methylcyclohexyl)amino)-6- morpholinopyridin-4-yl)-4- methylphenyl)-3-(2,2,2- trifluoroethyl)pyrrolidine-1-carboxamide |
| 38 | 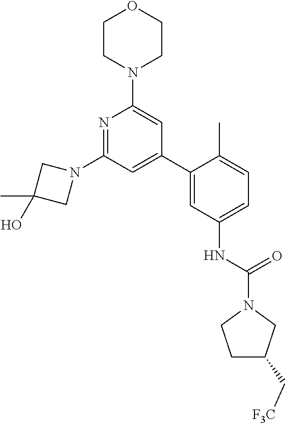 [SMILES: Cc1ccc(NC(=O)N2CC[C@@H](CC(F)(F)F)C2)cc1-c1cc(N2CCOCC2)nc(N2CC(C)(O)C2)c1] | (S)-N-(3-(2-(3-hydroxy-3- methylazetidin-1-yl)-6- morpholinopyridin-4-yl)-4- methylphenyl)-3-(2,2,2- trifluoroethyl)pyrrolidine-1-carboxamide |
| 39 | 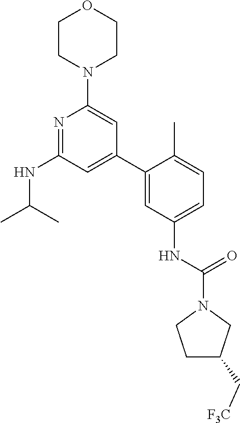 [SMILES: Cc1ccc(NC(=O)N2CC[C@@H](CC(F)(F)F)C2)cc1-c1cc(NC(C)C)nc(N2CCOCC2)c1] | (S)-N-(3-(2-(isopropylamino)-6- morpholinopyridin-4-yl)-4- methylphenyl)-3-(2,2,2- trifluoroethyl)pyrrolidine-1-carboxamide |
| 40 | 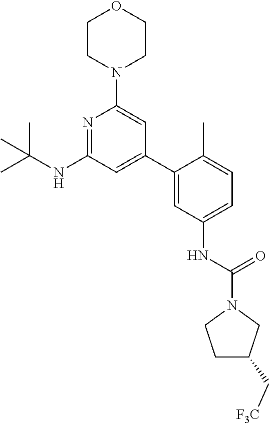 [SMILES: Cc1ccc(NC(=O)N2CC[C@@H](CC(F)(F)F)C2)cc1-c1cc(NC(C)(C)C)nc(N2CCOCC2)c1] | (S)-N-(3-(2-(tert-butylamino)-6- morpholinopyridin-4-yl)-4- methylphenyl)-3-(2,2,2- trifluoroethyl)pyrrolidine-1-carboxamide |
| 41 | 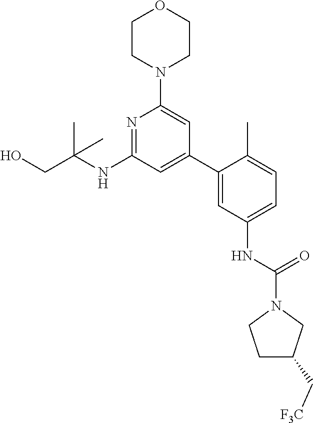 [SMILES: Cc1ccc(NC(=O)N2CC[C@@H](CC(F)(F)F)C2)cc1-c1cc(NC(C)(C)CO)nc(N2CCOCC2)c1] | (S)-N-(3-(2-((1-hydroxy-2-methylpropan- 2-yl)amino)-6-morpholinopyridin-4-yl)- 4-methylphenyl)-3-(2,2,2- trifluoroethyl)pyrrolidine-1-carboxamide |
| 42 | 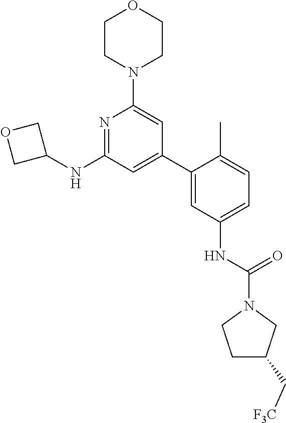 [SMILES: Cc1ccc(NC(=O)N2CC[C@@H](CC(F)(F)F)C2)cc1-c1cc(NC2COC2)nc(N2CCOCC2)c1] | (S)-N-(4-methyl-3-(2-morpholino-6- (oxetan-3-ylamino)pyridin-4-yl)phenyl)- 3-(2,2,2-trifluoroethyl)pyrrolidine-1- carboxamide |
| 43 | 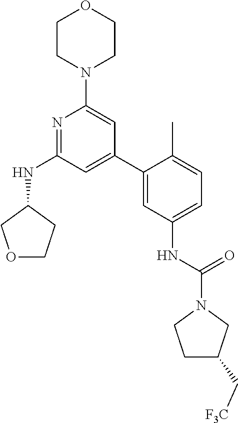 [SMILES: Cc1ccc(NC(=O)N2CC[C@@H](CC(F)(F)F)C2)cc1-c1cc(N[C@@H]2CCOC2)nc(N2CCOCC2)c1] | (S)-N-(4-methyl-3-(2-morpholino-6- (((R)-tetrahydrofuran-3- yl)amino)pyridin-4-yl)phenyl)-3-(2,2,2- trifluoroethyl)pyrrolidine-1-carboxamide |
| 44 | 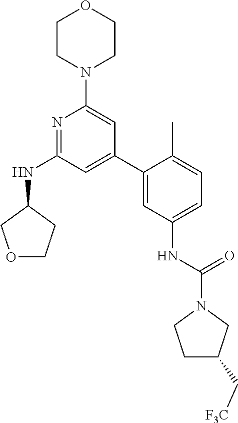 [SMILES: Cc1ccc(NC(=O)N2CC[C@@H](CC(F)(F)F)C2)cc1-c1cc(N[C@H]2CCOC2)nc(N2CCOCC2)c1] | (S)-N-(4-methyl-3-(2-morpholino-6- (((S)-tetrahydrofuran-3-yl)amino)pyridin- 4-yl)phenyl)-3-(2,2,2- trifluoroethyl)pyrrolidine-1-carboxamide |
| 45 | 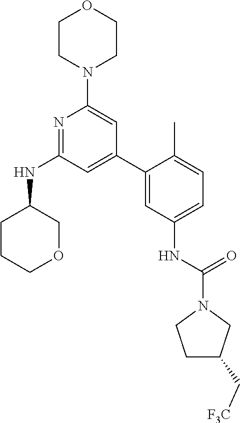 [SMILES: Cc1ccc(NC(=O)N2CC[C@@H](CC(F)(F)F)C2)cc1-c1cc(N[C@@H]2CCCOC2)nc(N2CCOCC2)c1] | (S)-N-(4-methyl-3-(2-morpholino-6- (((R)-tetrahydro-2H-pyran-3- yl)amino)pyridin-4-yl)phenyl)-3-(2,2,2- trifluoroethyl)pyrrolidine-1-carboxamide |
| 46 | 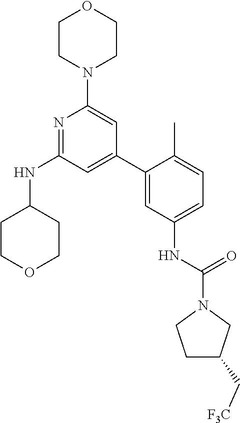 [SMILES: Cc1ccc(NC(=O)N2CC[C@@H](CC(F)(F)F)C2)cc1-c1cc(NC2CCOCC2)nc(N2CCOCC2)c1] | (S)-N-(4-methyl-3-(2-morpholino-6- ((tetrahydro-2H-pyran-4- yl)amino)pyridin-4-yl)phenyl)-3-(2,2,2- trifluoroethyl)pyrrolidine-1-carboxamide |
| 47 | 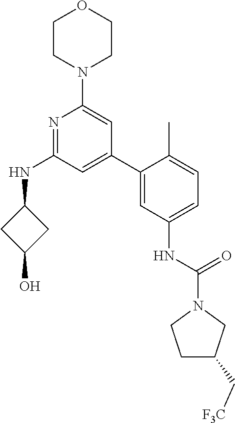 [SMILES: Cc1ccc(NC(=O)N2CC[C@@H](CC(F)(F)F)C2)cc1-c1cc(N[C@H]2C[C@@H](O)C2)nc(N2CCOCC2)c1] | (S)-N-(3-(2-(((1S,3R)-3- hydroxycyclobutyl)amino)-6- morpholinopyridin-4-yl)-4- methylphenyl)-3-(2,2,2- trifluoroethyl)pyrrolidine-1-carboxamide |
| 48 | 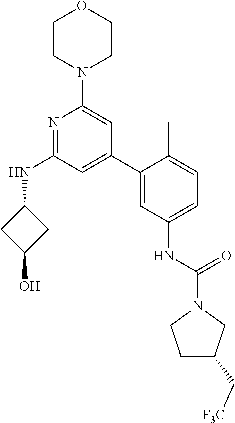 [SMILES: Cc1ccc(NC(=O)N2CC[C@@H](CC(F)(F)F)C2)cc1-c1cc(N[C@H]2C[C@H](O)C2)nc(N2CCOCC2)c1] | (S)-N-(3-(2-(((1R,3S)-3- hydroxycyclobutyl)amino)-6- morpholinopyridin-4-yl)-4- methylphenyl)-3-(2,2,2- trifluoroethyl)pyrrolidine-1-carboxamide |
| 49 | 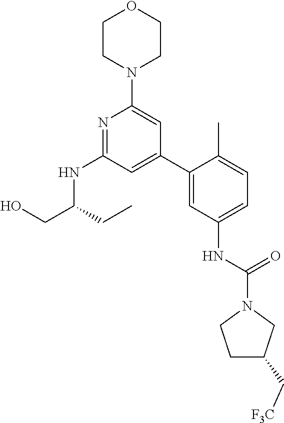 [SMILES: CC[C@H](CO)Nc1cc(-c2cc(NC(=O)N3CC[C@@H](CC(F)(F)F)C3)ccc2C)cc(N2CCOCC2)n1] | (S)-N-(3-(2-(((R)-1-hydroxybutan-2- yl)amino)-6-morpholinopyridin-4-yl)-4- methylphenyl)-3-(2,2,2- trifluoroethyl)pyrrolidine-1-carboxamide |
| 50 | 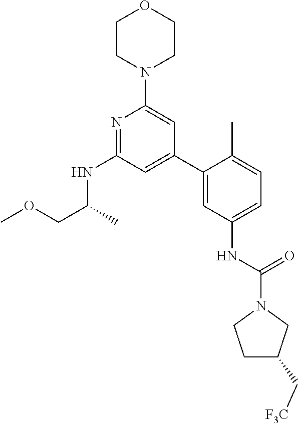 [SMILES: COC[C@@H](C)Nc1cc(-c2cc(NC(=O)N3CC[C@@H](CC(F)(F)F)C3)ccc2C)cc(N2CCOCC2)n1] | (S)-N-(3-(2-(((R)-1-methoxypropan-2- yl)amino)-6-morpholinopyridin-4-yl)-4- methylphenyl)-3-(2,2,2- trifluoroethyl)pyrrolidine-1-carboxamide |
| 51 | 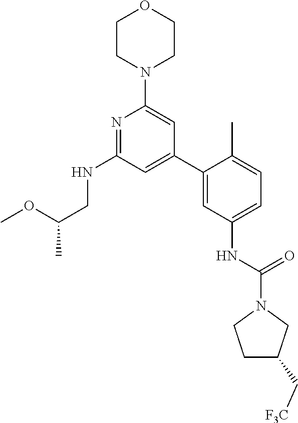 [SMILES: CO[C@@H](C)CNc1cc(-c2cc(NC(=O)N3CC[C@@H](CC(F)(F)F)C3)ccc2C)cc(N2CCOCC2)n1] | (S)-N-(3-(2-(((S)-2- methoxypropyl)amino)-6- morpholinopyridin-4-yl)-4- methylphenyl)-3-(2,2,2- trifluoroethyl)pyrrolidine-1-carboxamide |
| 52 | 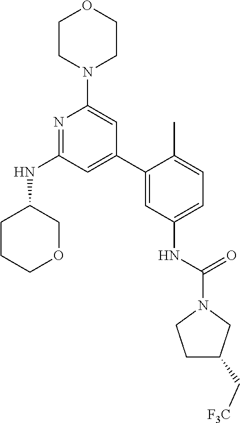 [SMILES: Cc1ccc(NC(=O)N2CC[C@@H](CC(F)(F)F)C2)cc1-c1cc(N[C@H]2CCCOC2)nc(N2CCOCC2)c1] | (S)-N-(4-methyl-3-(2-morpholino-6- (((S)-tetrahydro-2H-pyran-3- yl)amino)pyridin-4-yl)phenyl)-3-(2,2,2- trifluoroethyl)pyrrolidine-1-carboxamide |
| 53 | 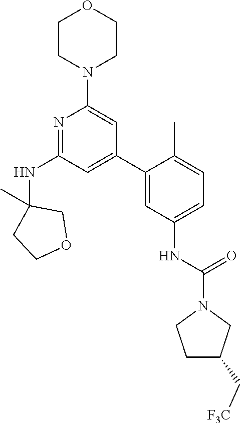 [SMILES: Cc1ccc(NC(=O)N2CC[C@@H](CC(F)(F)F)C2)cc1-c1cc(NC2(C)CCOC2)nc(N2CCOCC2)c1] | (3S)-N-(4-methyl-3-(2-((3- methyltetrahydrofuran-3-yl)amino)-6- morpholinopyridin-4-yl)phenyl)-3-(2,2,2- trifluoroethyl)pyrrolidine-1-carboxamide |
| 54 | 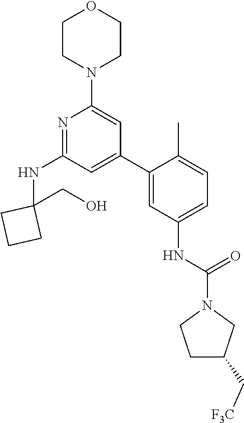 [SMILES: Cc1ccc(NC(=O)N2CC[C@@H](CC(F)(F)F)C2)cc1-c1cc(NC2(CO)CCC2)nc(N2CCOCC2)c1] | (S)-N-(3-(2-((1- (hydroxymethyl)cyclobutyl)amino)-6- morpholinopyridin-4-yl)-4- methylphenyl)-3-(2,2,2- trifluoroethyl)pyrrolidine-1-carboxamide |
| 55 | 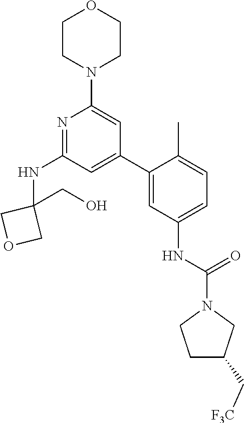 [SMILES: Cc1ccc(NC(=O)N2CC[C@@H](CC(F)(F)F)C2)cc1-c1cc(NC2(CO)COC2)nc(N2CCOCC2)c1] | (S)-N-(3-(2-((3-(hydroxymethyl)oxetan- 3-yl)amino)-6-morpholinopyridin-4-yl)- 4-methylphenyl)-3-(2,2,2- trifluoroethyl)pyrrolidine-1-carboxamide |
| 56 | 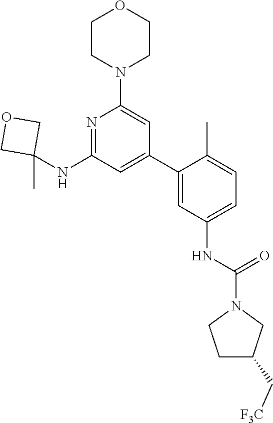 [SMILES: Cc1ccc(NC(=O)N2CC[C@@H](CC(F)(F)F)C2)cc1-c1cc(NC2(C)COC2)nc(N2CCOCC2)c1] | (S)-N-(4-methyl-3-(2-((3-methyloxetan- 3-yl)amino)-6-morpholinopyridin-4- yl)phenyl)-3-(2,2,2- trifluoroethyl)pyrrolidine-1-carboxamide |
| 57 | 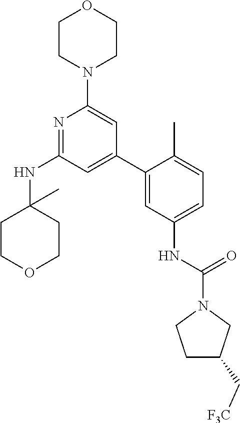 [SMILES: Cc1ccc(NC(=O)N2CC[C@@H](CC(F)(F)F)C2)cc1-c1cc(NC2(C)CCOCC2)nc(N2CCOCC2)c1] | (S)-N-(4-methyl-3-(2-((4- methyltetrahydro-2H-pyran-4-yl)amino)- 6-morpholinopyridin-4-yl)phenyl)-3- (2,2,2-trifluoroethyl)pyrrolidine-1- carboxamide |
| 58 | 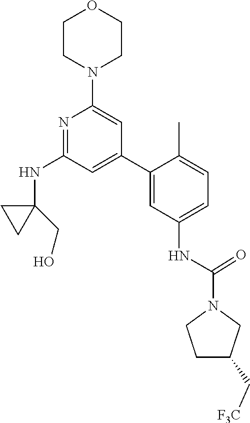 [SMILES: Cc1ccc(NC(=O)N2CC[C@@H](CC(F)(F)F)C2)cc1-c1cc(NC2(CO)CC2)nc(N2CCOCC2)c1] | (S)-N-(3-(2-((1- (hydroxymethyl)cyclopropyl)amino)-6- morpholinopyridin-4-yl)-4- methylphenyl)-3-(2,2,2- trifluoroethyl)pyrrolidine-1-carboxamide |
| 59 and 60 | 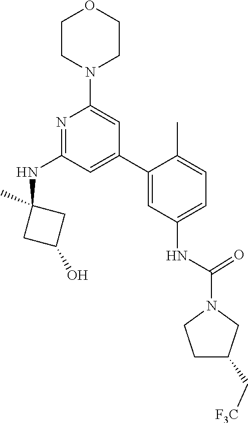 [SMILES: Cc1ccc(NC(=O)N2CC[C@@H](CC(F)(F)F)C2)cc1-c1cc(N[C@]2(C)C[C@@H](O)C2)nc(N2CCOCC2)c1] | (S)-N-(3-(2-(((1R,3S)-3-hydroxy-1- methylcyclobutyl)amino)-6- morpholinopyridin-4-yl)-4- methylphenyl)-3-(2,2,2- trifluoroethyl)pyrrolidine-1-carboxamide and (S)-N-(3-(2-(((1S,3R)-3-hydroxy-1- methylcyclobutyl)amino)-6- morpholinopyridin-4-yl)-4- methylphenyl)-3-(2,2,2- trifluoroethyl)pyrrolidine-1-carboxamide |
| | + | |
| | 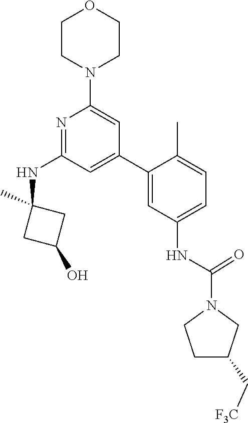 [SMILES: Cc1ccc(NC(=O)N2CC[C@@H](CC(F)(F)F)C2)cc1-c1cc(N[C@]2(C)C[C@H](O)C2)nc(N2CCOCC2)c1] | |
| 61 | 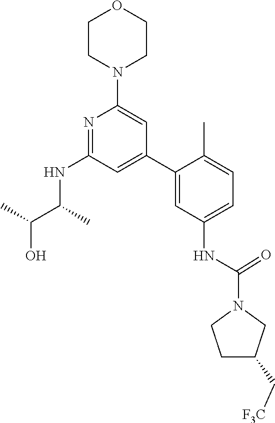 [SMILES: Cc1ccc(NC(=O)N2CC[C@@H](CC(F)(F)F)C2)cc1-c1cc(N[C@H](C)[C@@H](C)O)nc(N2CCOCC2)c1] | (S)-N-(3-(2-(((2R,3R)-3-hydroxybutan-2- yl)amino)-6-morpholinopyridin-4-yl)-4- methylphenyl)-3-(2,2,2- trifluoroethyl)pyrrolidine-1-carboxamide |
| 62 | 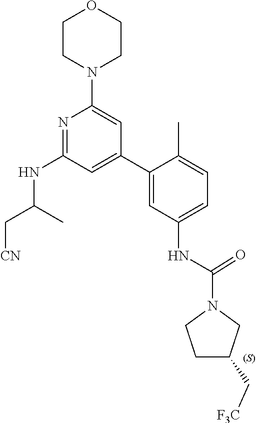 | (3S)-N-(3-(2-((1-cyanopropan-2- yl)amino)-6-morpholinopyridin-4-yl)-4- methylphenyl)-3-(2,2,2- trifluoroethyl)pyrrolidine-1-carboxamide |
| 63 and 64 | 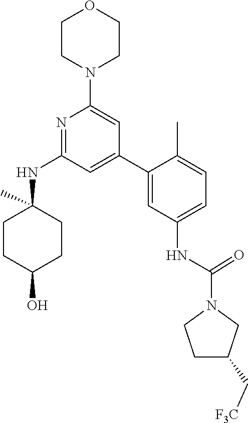 [SMILES: Cc1ccc(NC(=O)N2CC[C@@H](CC(F)(F)F)C2)cc1-c1cc(N[C@]2(C)CC[C@H](O)CC2)nc(N2CCOCC2)c1] | (S)-N-(3-(2-(((1S,4R)-4-hydroxy-1- methylcyclohexyl)amino)-6- morpholinopyridin-4-yl)-4- methylphenyl)-3-(2,2,2- trifluoroethyl)pyrrolidine-1-carboxamide And (S)-N-(3-(2-(((1R,4S)-4-hydroxy-1- methylcyclohexyl)amino)-6- morpholinopyridin-4-yl)-4- methylphenyl)-3-(2,2,2- trifluoroethyl)pyrrolidine-1-carboxamide |
| | + | |
| | 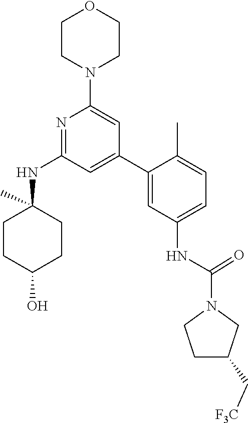 [SMILES: Cc1ccc(NC(=O)N2CC[C@@H](CC(F)(F)F)C2)cc1-c1cc(N[C@]2(C)CC[C@@H](O)CC2)nc(N2CCOCC2)c1] | |
| 65 | 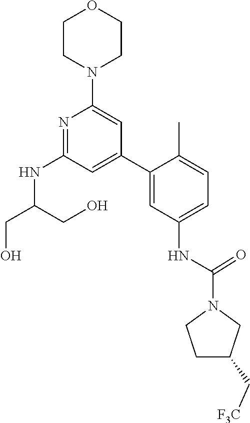 [SMILES: Cc1ccc(NC(=O)N2CC[C@@H](CC(F)(F)F)C2)cc1-c1cc(NC(CO)CO)nc(N2CCOCC2)c1] | (S)-N-(3-(2-((1,3-dihydroxypropan-2- yl)amino)-6-morpholinopyridin-4-yl)-4- methylphenyl)-3-(2,2,2- trifluoroethyl)pyrrolidine-1-carboxamide |
| 66 | 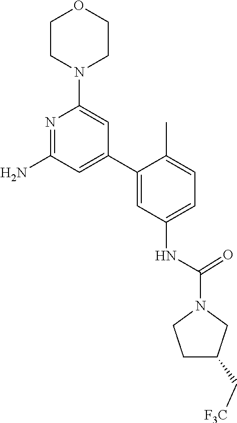 [SMILES: Cc1ccc(NC(=O)N2CC[C@@H](CC(F)(F)F)C2)cc1-c1cc(N)nc(N2CCOCC2)c1] | (S)-N-(3-(2-amino-6-morpholinopyridin- 4-yl)-4-methylphenyl)-3-(2,2,2- trifluoroethyl)pyrrolidine-1-carboxamide |
| 67 | 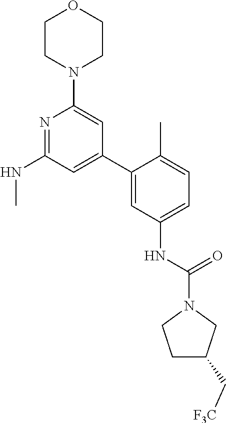 [SMILES: CNc1cc(-c2cc(NC(=O)N3CC[C@@H](CC(F)(F)F)C3)ccc2C)cc(N2CCOCC2)n1] | (S)-N-(4-methyl-3-(2-(methylamino)-6- morpholinopyridin-4-yl)phenyl)-3-(2,2,2- trifluoroethyl)pyrrolidine-1-carboxamide |
| 68 | 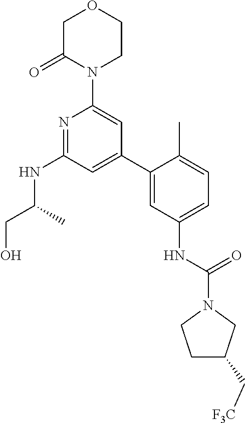 [SMILES: Cc1ccc(NC(=O)N2CC[C@@H](CC(F)(F)F)C2)cc1-c1cc(N[C@H](C)CO)nc(N2CCOCC2=O)c1] | (S)-N-(3-(2-(((R)-1-hydroxypropan-2- yl)amino)-6-(3-oxomorpholino)pyridin- 4-yl)-4-methylphenyl)-3-(2,2,2- trifluoroethyl)pyrrolidine-1-carboxamide |
| 69 | 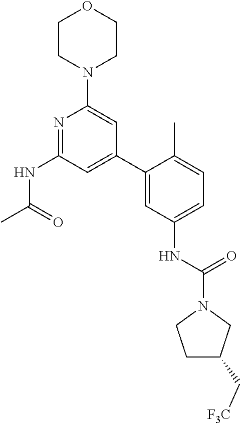 [SMILES: CC(=O)Nc1cc(-c2cc(NC(=O)N3CC[C@@H](CC(F)(F)F)C3)ccc2C)cc(N2CCOCC2)n1] | (S)-N-(3-(2-acetamido-6- morpholinopyridin-4-yl)-4- methylphenyl)-3-(2,2,2- trifluoroethyl)pyrrolidine-1-carboxamide |
| 70 | 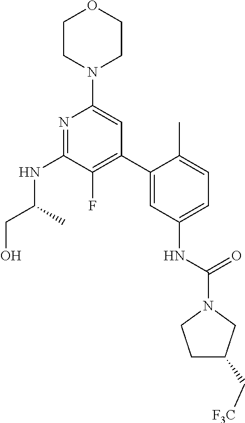 [SMILES: Cc1ccc(NC(=O)N2CC[C@@H](CC(F)(F)F)C2)cc1-c1cc(N2CCOCC2)nc(N[C@H](C)CO)c1F] | (S)-N-(3-(3-fluoro-2-(((R)-1- hydroxypropan-2-yl)amino)-6- morpholinopyridin-4-yl)-4- methylphenyl)-3-(2,2,2- trifluoroethyl)pyrrolidine-1-carboxamide |
| 71 | 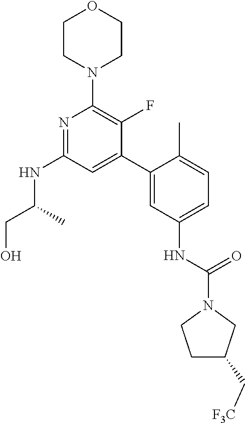 [SMILES: Cc1ccc(NC(=O)N2CC[C@@H](CC(F)(F)F)C2)cc1-c1cc(N[C@H](C)CO)nc(N2CCOCC2)c1F] | (S)-N-(3-(3-fluoro-6-(((R)-1- hydroxypropan-2-yl)amino)-2- morpholinopyridin-4-yl)-4- methylphenyl)-3-(2,2,2- trifluoroethyl)pyrrolidine-1-carboxamide |
| 72 and 73 | 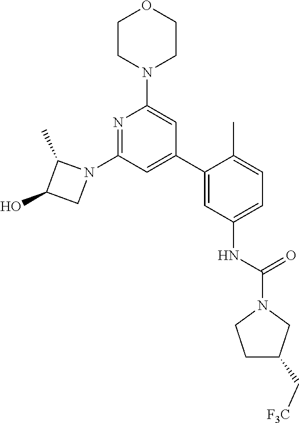 [SMILES: Cc1ccc(NC(=O)N2CC[C@@H](CC(F)(F)F)C2)cc1-c1cc(N2CCOCC2)nc(N2C[C@@H](O)[C@@H]2C)c1] | (S)-N-(3-(2-((2S,3R)-3-hydroxy-2- methylazetidin-1-yl)-6- morpholinopyridin-4-yl)-4- methylphenyl)-3-(2,2,2- trifluoroethyl)pyrrolidine-1-carboxamide and (S)-N-(3-(2-((2S,3S)-3-hydroxy-2- methylazetidin-1-yl)-6- morpholinopyridin-4-yl)-4- methylphenyl)-3-(2,2,2- trifluoroethyl)pyrrolidine-1-carboxamide |
| | + | |
| | 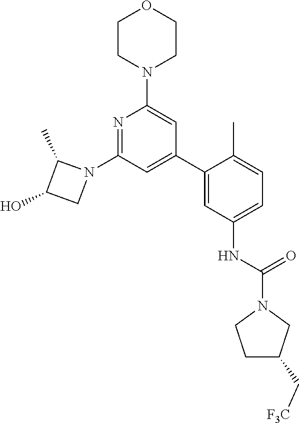 [SMILES: Cc1ccc(NC(=O)N2CC[C@@H](CC(F)(F)F)C2)cc1-c1cc(N2CCOCC2)nc(N2C[C@H](O)[C@@H]2C)c1] | |
| 74 | 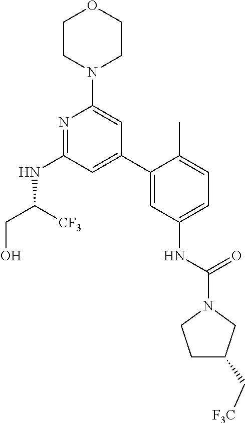 [SMILES: Cc1ccc(NC(=O)N2CC[C@@H](CC(F)(F)F)C2)cc1-c1cc(N[C@@H](CO)C(F)(F)F)nc(N2CCOCC2)c1] | (S)-N-(4-methyl-3-(2-morpholino-6- (((S)-1,1,1-trifluoro-3-hydroxypropan-2- yl)amino)pyridin-4-yl)phenyl)-3-(2,2,2- trifluoroethyl)pyrrolidine-1-carboxamide |
| 75 | 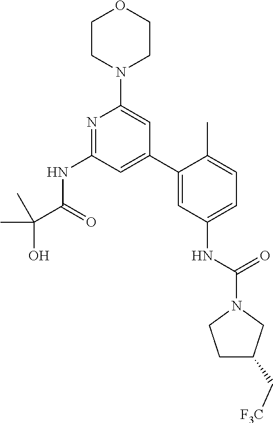 [SMILES: Cc1ccc(NC(=O)N2CC[C@@H](CC(F)(F)F)C2)cc1-c1cc(NC(=O)C(C)(C)O)nc(N2CCOCC2)c1] | (S)-N-(3-(2-(2-hydroxy-2- methylpropanamido)-6- morpholinopyridin-4-yl)-4- methylphenyl)-3-(2,2,2- trifluoroethyl)pyrrolidine-1-carboxamide |
| 76 | 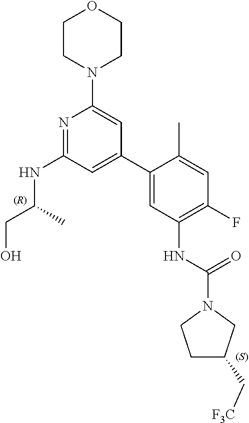 | (S)-N-(2-fluoro-5-(2-(((R)-1- hydroxypropan-2-yl)amino)-6- morpholinopyridin-4-yl)-4- methylphenyl)-3-(2,2,2- trifluoroethyl)pyrrolidine-1-carboxamide |
| 77 | 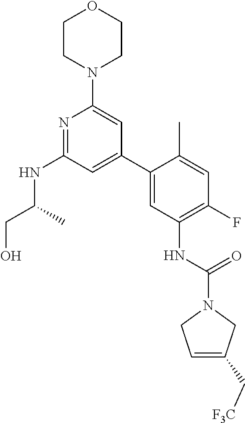 | (R)-N-(2-fluoro-5-(2-((1-hydroxypropan- 2-yl)amino)-6-morpholinopyridin-4-yl)- 4-methylphenyl)-3-(2,2,2-trifluoroethyl)- 2,5-dihydro-1H-pyrrole-1-carboxamide |
| 78 | 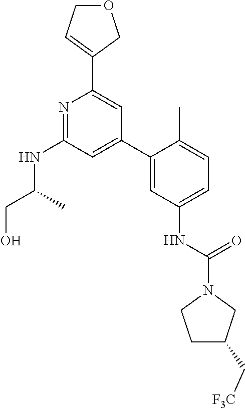 [SMILES: Cc1ccc(NC(=O)N2CC[C@@H](CC(F)(F)F)C2)cc1-c1cc(N[C@H](C)CO)nc(C2=CCOC2)c1] | (S)-N-(3-(2-(2,5-dihydrofuran-3-yl)-6- (((R)-1-hydroxypropan-2- yl)amino)pyridin-4-yl)-4-methylphenyl)- 3-(2,2,2-trifluoroethyl)pyrrolidine-1- carboxamide |
| 79 | 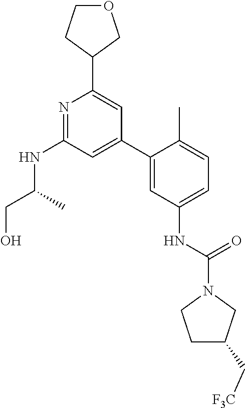 [SMILES: Cc1ccc(NC(=O)N2CC[C@@H](CC(F)(F)F)C2)cc1-c1cc(N[C@H](C)CO)nc(C2CCOC2)c1] | (3S)-N-(3-(2-(((R)-1-hydroxypropan-2- yl)amino)-6-(tetrahydrofuran-3- yl)pyridin-4-yl)-4-methylphenyl)-3- (2,2,2-trifluoroethyl)pyrrolidine-1- carboxamide |
| 80 | 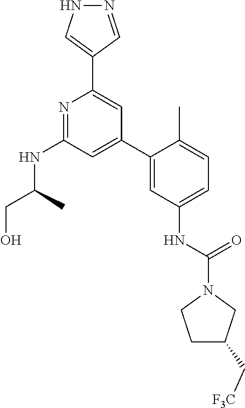 [SMILES: Cc1ccc(NC(=O)N2CC[C@@H](CC(F)(F)F)C2)cc1-c1cc(N[C@@H](C)CO)nc(-c2cn[nH]c2)c1] | (S)-N-(3-(2-(((S)-1-hydroxypropan-2- yl)amino)-6-(1H-pyrazol-4-yl)pyridin-4- yl)-4-methylphenyl)-3-(2,2,2- trifluoroethyl)pyrrolidine-1-carboxamide |
| 81 | 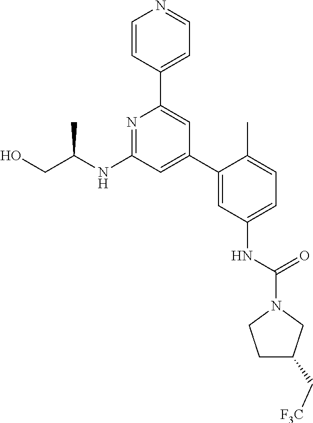 [SMILES: Cc1ccc(NC(=O)N2CC[C@@H](CC(F)(F)F)C2)cc1-c1cc(N[C@H](C)CO)nc(-c2ccncc2)c1] | (S)-N-(3-(6-(((R)-1-hydroxypropan-2- yl)amino)-[2,4′-bipyridin]-4-yl)-4- methylphenyl)-3-(2,2,2- trifluoroethyl)pyrrolidine-1-carboxamide |
| 82 | 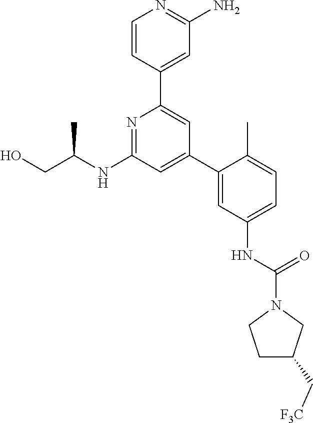 [SMILES: Cc1ccc(NC(=O)N2CC[C@@H](CC(F)(F)F)C2)cc1-c1cc(N[C@H](C)CO)nc(-c2ccnc(N)c2)c1] | (S)-N-(3-(2′-amino-6-(((R)-1- hydroxypropan-2-yl)amino)-[2,4′- bipyridin]-4-yl)-4-methylphenyl)-3- (2,2,2-trifluoroethyl)pyrrolidine-1- carboxamide |
| 83 | 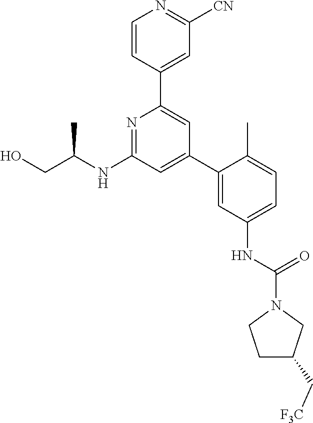 [SMILES: Cc1ccc(NC(=O)N2CC[C@@H](CC(F)(F)F)C2)cc1-c1cc(N[C@H](C)CO)nc(-c2ccnc(C#N)c2)c1] | (S)-N-(3-(2′-cyano-6-(((R)-1- hydroxypropan-2-yl)amino)-[2,4′- bipyridin]-4-yl)-4-methylphenyl)-3- (2,2,2-trifluoroethyl)pyrrolidine-1- carboxamide |
| 84 and 85 | 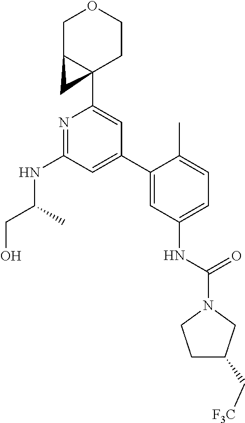 [SMILES: Cc1ccc(NC(=O)N2CC[C@@H](CC(F)(F)F)C2)cc1-c1cc(N[C@H](C)CO)nc([C@@]23CCOC[C@@H]2C3)c1] | (S)-N-(3-(2-((1R,6S)-3- oxabicyclo[4.1.0]heptan-6-yl)-6-(((R)-1- hydroxypropan-2-yl)amino)pyridin-4-yl)- 4-methylphenyl)-3-(2,2,2- trifluoroethyl)pyrrolidine-1-carboxamide and (S)-N-(3-(2-((1S,6R)-3- oxabicyclo[4.1.0]heptan-6-yl)-6-(((R)-1- hydroxypropan-2-yl)amino)pyridin-4-yl)- 4-methylphenyl)-3-(2,2,2- trifluoroethyl)pyrrolidine-1-carboxamide |
| | + | |
| | 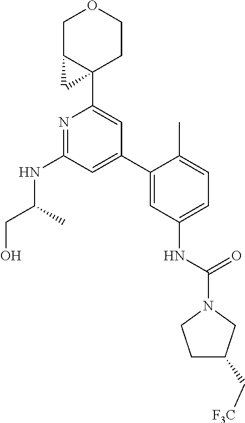 [SMILES: Cc1ccc(NC(=O)N2CC[C@@H](CC(F)(F)F)C2)cc1-c1cc(N[C@H](C)CO)nc([C@]23CCOC[C@H]2C3)c1] | |
| 86 | 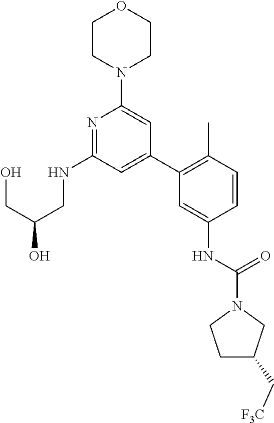 [SMILES: Cc1ccc(NC(=O)N2CC[C@@H](CC(F)(F)F)C2)cc1-c1cc(NC[C@@H](O)CO)nc(N2CCOCC2)c1] | (S)-N-(3-(2-(((R)-2,3- dihydroxypropyl)amino)-6- morpholinopyridin-4-yl)-4- methylphenyl)-3-(2,2,2- trifluoroethyl)pyrrolidine-1-carboxamide |
| 87 | 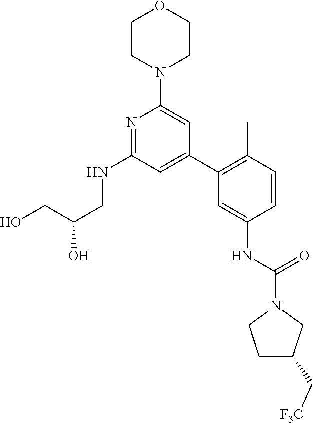 [SMILES: Cc1ccc(NC(=O)N2CC[C@@H](CC(F)(F)F)C2)cc1-c1cc(NC[C@H](O)CO)nc(N2CCOCC2)c1] | (S)-N-(3-(2-(((S)-2,3- dihydroxypropyl)amino)-6- morpholinopyridin-4-yl)-4- methylphenyl)-3-(2,2,2- trifluoroethyl)pyrrolidine-1-carboxamide |
| 88 and 89 | 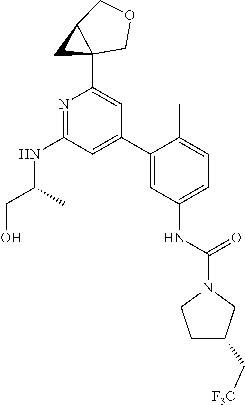 [SMILES: Cc1ccc(NC(=O)N2CC[C@@H](CC(F)(F)F)C2)cc1-c1cc(N[C@H](C)CO)nc([C@@]23COC[C@@H]2C3)c1] | (S)-N-(3-(2-((1S,5R)-3- oxabicyclo[3.1.0]hexan-1-yl)-6-(((R)-1- hydroxypropan-2-yl)amino)pyridin-4-yl)- 4-methylphenyl)-3-(2,2,2- trifluoroethyl)pyrrolidine-1-carboxamide and (S)-N-(3-(2-((1R,5S)-3- oxabicyclo[3.1.0]hexan-1-yl)-6-(((R)-1- hydroxypropan-2-yl)amino)pyridin-4-yl)- 4-methylphenyl)-3-(2,2,2- trifluoroethyl)pyrrolidine-1-carboxamide |
| | + | |
| | 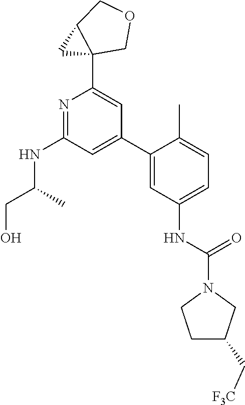 [SMILES: Cc1ccc(NC(=O)N2CC[C@@H](CC(F)(F)F)C2)cc1-c1cc(N[C@H](C)CO)nc([C@]23COC[C@H]2C3)c1] | |
| 90 and 91 | 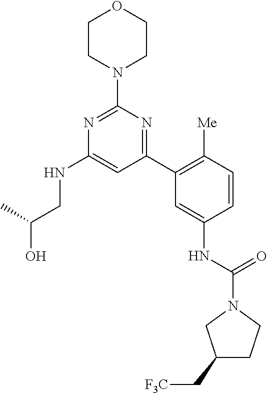 [SMILES: Cc1ccc(NC(=O)N2CC[C@@H](CC(F)(F)F)C2)cc1-c1cc(NC[C@@H](C)O)nc(N2CCOCC2)n1] | (S)-N-(3-(6-(((R)-2- hydroxypropyl)amino)-2- morpholinopyrimidin-4-yl)-4- methylphenyl)-3-(2,2,2- trifluoroethyl)pyrrolidine-1-carboxamide and (R)-N-(3-(6-(((R)-2- hydroxypropyl)amino)-2- morpholinopyrimidin-4-yl)-4- methylphenyl)-3-(2,2,2- trifluoroethyl)pyrrolidine-1-carboxamide |
| | + | |
| | 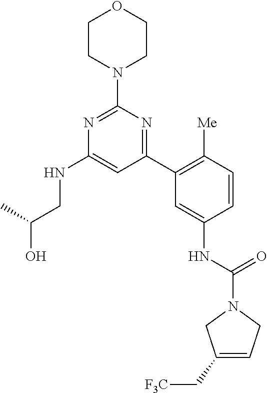 | |
| 92 | 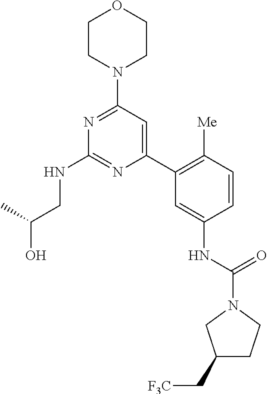 [SMILES: Cc1ccc(NC(=O)N2CC[C@@H](CC(F)(F)F)C2)cc1-c1cc(N2CCOCC2)nc(NC[C@@H](C)O)n1] | (S)-N-(3-(2-(((R)-2- hydroxypropyl)amino)-6- morpholinopyrimidin-4-yl)-4- methylphenyl)-3-(2,2,2- trifluoroethyl)pyrrolidine-1-carboxamide |
| 93 | 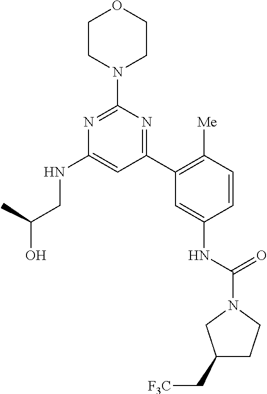 [SMILES: Cc1ccc(NC(=O)N2CC[C@@H](CC(F)(F)F)C2)cc1-c1cc(NC[C@H](C)O)nc(N2CCOCC2)n1] | (S)-N-(3-(6-(((S)-2- hydroxypropyl)amino)-2- morpholinopyrimidin-4-yl)-4- methylphenyl)-3-(2,2,2- trifluoroethyl)pyrrolidine-1-carboxamide |
| 94 | 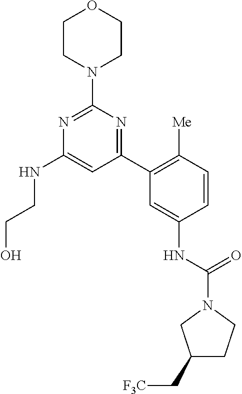 [SMILES: Cc1ccc(NC(=O)N2CC[C@@H](CC(F)(F)F)C2)cc1-c1cc(NCCO)nc(N2CCOCC2)n1] | (S)-N-(3-(6-((2-hydroxyethyl)amino)-2- morpholinopyrimidin-4-yl)-4- methylphenyl)-3-(2,2,2- trifluoroethyl)pyrrolidine-1-carboxamide |
| 95 | 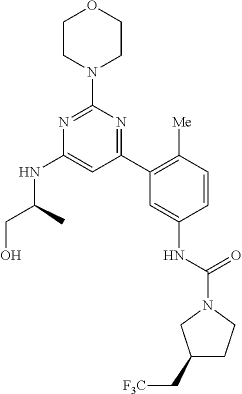 [SMILES: Cc1ccc(NC(=O)N2CC[C@@H](CC(F)(F)F)C2)cc1-c1cc(N[C@@H](C)CO)nc(N2CCOCC2)n1] | (S)-N-(3-(6-(((S)-1-hydroxypropan-2- yl)amino)-2-morpholinopyrimidin-4-yl)- 4-methylphenyl)-3-(2,2,2- trifluoroethyl)pyrrolidine-1-carboxamide |
| 96 | 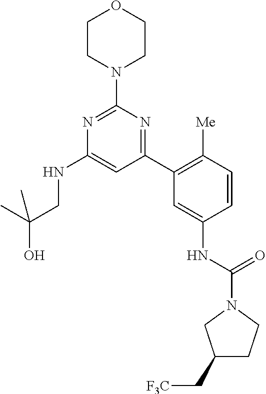 [SMILES: Cc1ccc(NC(=O)N2CC[C@@H](CC(F)(F)F)C2)cc1-c1cc(NCC(C)(C)O)nc(N2CCOCC2)n1] | (S)-N-(3-(6-((2-hydroxy-2- methylpropyl)amino)-2- morpholinopyrimidin-4-yl)-4- methylphenyl)-3-(2,2,2- trifluoroethyl)pyrrolidine-1-carboxamide |
| 97 | 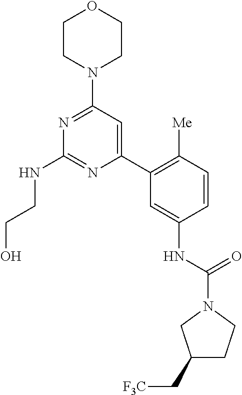 [SMILES: Cc1ccc(NC(=O)N2CC[C@@H](CC(F)(F)F)C2)cc1-c1cc(N2CCOCC2)nc(NCCO)n1] | (S)-N-(3-(2-((2-hydroxyethyl)amino)-6- morpholinopyrimidin-4-yl)-4- methylphenyl)-3-(2,2,2- trifluoroethyl)pyrrolidine-1-carboxamide |
| 98 | 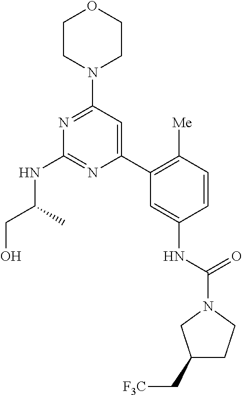 [SMILES: Cc1ccc(NC(=O)N2CC[C@@H](CC(F)(F)F)C2)cc1-c1cc(N2CCOCC2)nc(N[C@H](C)CO)n1] | (S)-N-(3-(2-(((R)-1-hydroxypropan-2- yl)amino)-6-morpholinopyrimidin-4-yl)- 4-methylphenyl)-3-(2,2,2- trifluoroethyl)pyrrolidine-1-carboxamide |
| 99 | 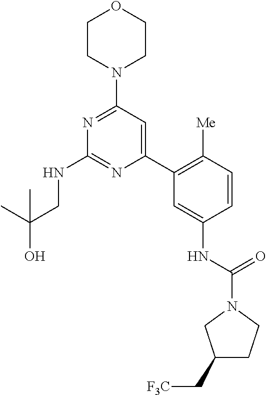 [SMILES: Cc1ccc(NC(=O)N2CC[C@@H](CC(F)(F)F)C2)cc1-c1cc(N2CCOCC2)nc(NCC(C)(C)O)n1] | (S)-N-(3-(2-((2-hydroxy-2- methylpropyl)amino)-6- morpholinopyrimidin-4-yl)-4- methylphenyl)-3-(2,2,2- trifluoroethyl)pyrrolidine-1-carboxamide |
| 100 | 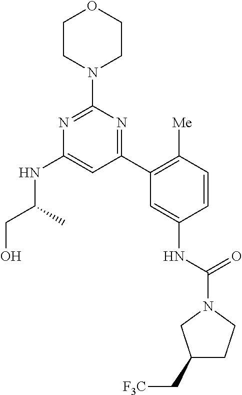 [SMILES: Cc1ccc(NC(=O)N2CC[C@@H](CC(F)(F)F)C2)cc1-c1cc(N[C@H](C)CO)nc(N2CCOCC2)n1] | (S)-N-(3-(6-(((R)-1-hydroxypropan-2- yl)amino)-2-morpholinopyrimidin-4-yl)- 4-methylphenyl)-3-(2,2,2- trifluoroethyl)pyrrolidine-1-carboxamide |
| 101 | 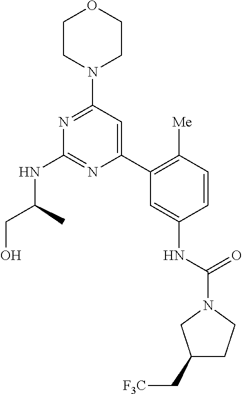 [SMILES: Cc1ccc(NC(=O)N2CC[C@@H](CC(F)(F)F)C2)cc1-c1cc(N2CCOCC2)nc(N[C@@H](C)CO)n1] | (S)-N-(3-(2-(((S)-1-hydroxypropan-2- yl)amino)-6-morpholinopyrimidin-4-yl)- 4-methylphenyl)-3-(2,2,2- trifluoroethyl)pyrrolidine-1-carboxamide |
| 102 and 103 | 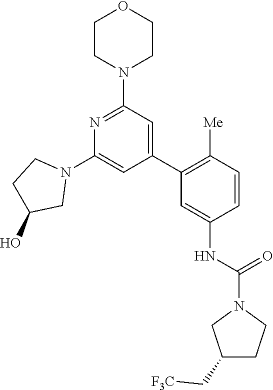 [SMILES: Cc1ccc(NC(=O)N2CC[C@H](CC(F)(F)F)C2)cc1-c1cc(N2CCOCC2)nc(N2CC[C@H](O)C2)c1] | (R)-N-(3-(2-((S)-3-hydroxypyrrolidin-1- yl)-6-morpholinopyridin-4-yl)-4- methylphenyl)-3-(2,2,2- trifluoroethyl)pyrrolidine-1-carboxamide and (S)-N-(3-(2-((S)-3-hydroxypyrrolidin-1- yl)-6-morpholinopyridin-4-yl)-4- methylphenyl)-3-(2,2,2- trifluoroethyl)pyrrolidine-1-carboxamide |
| | + | |
| | 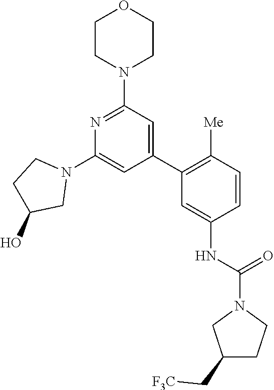 [SMILES: Cc1ccc(NC(=O)N2CC[C@@H](CC(F)(F)F)C2)cc1-c1cc(N2CCOCC2)nc(N2CC[C@H](O)C2)c1] | |
| 104 | 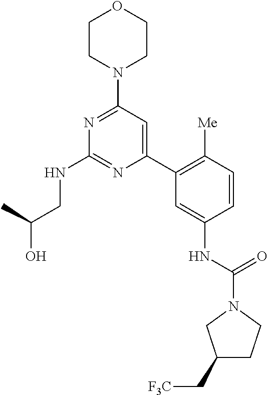 [SMILES: Cc1ccc(NC(=O)N2CC[C@@H](CC(F)(F)F)C2)cc1-c1cc(N2CCOCC2)nc(NC[C@H](C)O)n1] | (S)-N-(3-(2-(((S)-2- hydroxypropyl)amino)-6- morpholinopyrimidin-4-yl)-4- methylphenyl)-3-(2,2,2- trifluoroethyl)pyrrolidine-1-carboxamide |
| 105 | 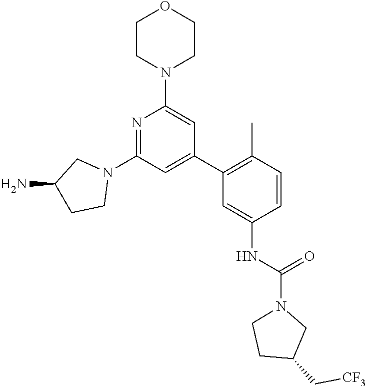 [SMILES: Cc1ccc(NC(=O)N2CC[C@@H](CC(F)(F)F)C2)cc1-c1cc(N2CCOCC2)nc(N2CC[C@@H](N)C2)c1] | (3S)-N-(3-[2-[(3R)-3-aminopyrrolidin-1- yl]-6-(morpholin-4-yl)pyridin-4-yl]-4- methylphenyl)-3-(2,2,2- trifluoroethyl)pyrrolidine-1-carboxamide |
| 106 | 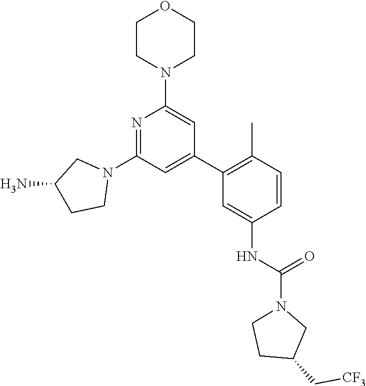 [SMILES: Cc1ccc(NC(=O)N2CC[C@@H](CC(F)(F)F)C2)cc1-c1cc(N2CCOCC2)nc(N2CC[C@H](N)C2)c1] | (3S)-N-(3-[2-[(3S)-3-aminopyrrolidin-1- yl]-6-(morpholin-4-yl)pyridin-4-yl]-4- methylphenyl)-3-(2,2,2- trifluoroethyl)pyrrolidine-1-carboxamide |
| 107 | 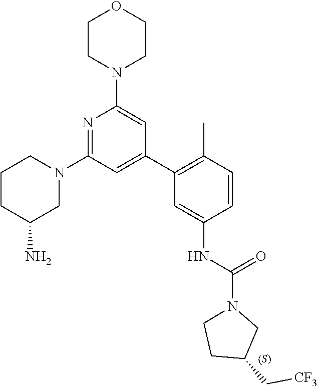 | (3S)-N-(3-[2-[(3R)-3-aminopiperidin-1- yl]-6-(morpholin-4-yl)pyridin-4-yl]-4- methylphenyl)-3-(2,2,2- trifluoroethyl)pyrrolidine-1-carboxamide |
| 108 | 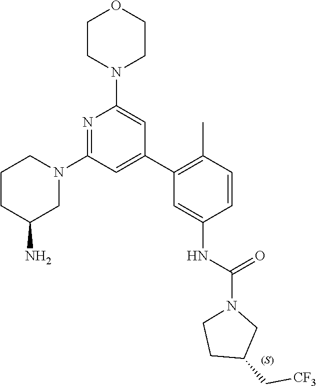 | (3S)-N-(3-[2-[(3S)-3-aminopiperidin-1- yl]-6-(morpholin-4-yl)pyridin-4-yl]-4- methylphenyl)-3-(2,2,2- trifluoroethyl)pyrrolidine-1-carboxamide |
| 109 | 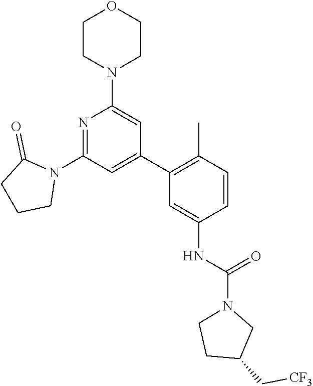 [SMILES: Cc1ccc(NC(=O)N2CC[C@@H](CC(F)(F)F)C2)cc1-c1cc(N2CCOCC2)nc(N2CCCC2=O)c1] | (3S)-N-[4-methyl-3-[2-(morpholin-4-yl)- 6-(2-oxopyrrolidin-1-yl)pyridin-4- yl]phenyl]-3-(2,2,2- trifluoroethyl)pyrrolidine-1-carboxamide |
| 110 | 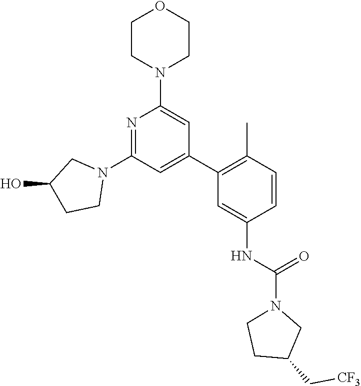 [SMILES: Cc1ccc(NC(=O)N2CC[C@@H](CC(F)(F)F)C2)cc1-c1cc(N2CCOCC2)nc(N2CC[C@@H](O)C2)c1] | (3S)-N-(3-[2-[(3R)-3-hydroxypyrrolidin- 1-yl]-6-(morpholin-4-yl)pyridin-4-yl]-4- methylphenyl)-3-(2,2,2- trifluoroethyl)pyrrolidine-1-carboxamide |
| 111 | 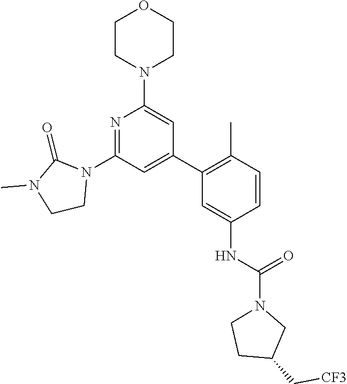 [SMILES: Cc1ccc(NC(=O)N2CC[C@@H](CC(F)(F)F)C2)cc1-c1cc(N2CCOCC2)nc(N2CCN(C)C2=O)c1] | (3S)-N-[4-methyl-3-[2-(3-methyl-2- oxoimidazolidin-1-yl)-6-(morpholin-4- yl)pyridin-4-yl]phenyl]-3-(2,2,2- trifluoroethyl)pyrrolidine-1-carboxamide |
| 112 | 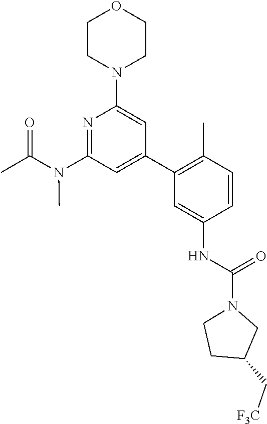 [SMILES: CC(=O)N(C)c1cc(-c2cc(NC(=O)N3CC[C@@H](CC(F)(F)F)C3)ccc2C)cc(N2CCOCC2)n1] | (3S)-N-[4-methyl-3-[2-(N- methylacetamido)-6-(morpholin-4- yl)pyridin-4-yl]phenyl]-3-(2,2,2- trifluoroethyl)pyrrolidine-1-carboxamide |
| 113 | 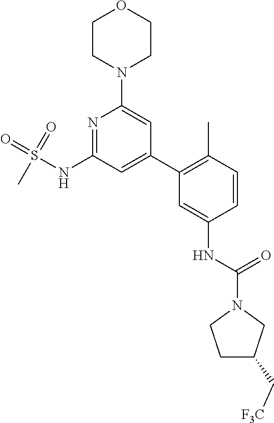 [SMILES: Cc1ccc(NC(=O)N2CC[C@@H](CC(F)(F)F)C2)cc1-c1cc(NS(C)(=O)=O)nc(N2CCOCC2)c1] | (3S)-N-[3-[2-methanesulfonamido-6- (morpholin-4-yl)pyridin-4-yl]-4- methylphenyl]-3-(2,2,2- trifluoroethyl)pyrrolidine-1-carboxamide |
| 114 | 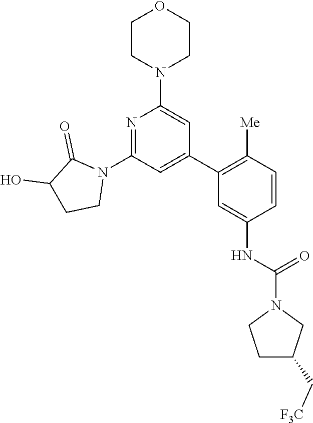 [SMILES: Cc1ccc(NC(=O)N2CC[C@@H](CC(F)(F)F)C2)cc1-c1cc(N2CCOCC2)nc(N2CCC(O)C2=O)c1] | (3S)-N-[3-[2-(3-hydroxy-2- oxopyrrolidin-1-yl)-6-(morpholin-4- yl)pyridin-4-yl]-4-methylphenyl]-3- (2,2,2-trifluoroethyl)pyrrolidine-1- carboxamide |
| 115 | 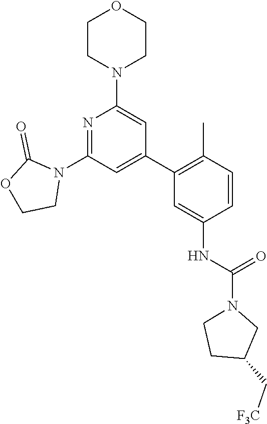 [SMILES: Cc1ccc(NC(=O)N2CC[C@@H](CC(F)(F)F)C2)cc1-c1cc(N2CCOCC2)nc(N2CCOC2=O)c1] | (3S)-N-[4-methyl-3-[2-(morpholin-4-yl)- 6-(2-oxo-1,3-oxazolidin-3-yl)pyridin-4- yl]phenyl]-3-(2,2,2- trifluoroethyl)pyrrolidine-1-carboxamide |
| 116 | 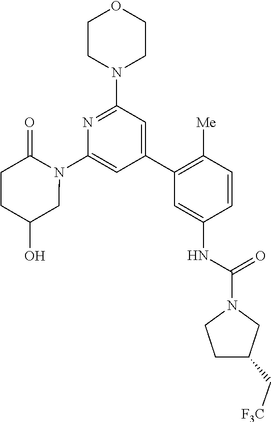 [SMILES: Cc1ccc(NC(=O)N2CC[C@@H](CC(F)(F)F)C2)cc1-c1cc(N2CCOCC2)nc(N2CC(O)CCC2=O)c1] | (3S)-N-(3-[2-[(5R)-5-hydroxy-2- oxopiperidin-1-yl]-6-(morpholin-4- yl)pyridin-4-yl]-4-methylphenyl)-3- (2,2,2-trifluoroethyl)pyrrolidine-1- carboxamide |
| 117 | 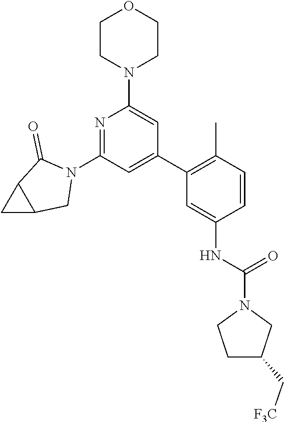 [SMILES: Cc1ccc(NC(=O)N2CC[C@@H](CC(F)(F)F)C2)cc1-c1cc(N2CCOCC2)nc(N2CC3CC3C2=O)c1] | (3S)-N-(4-methyl-3-(2-morpholino-6-(2- oxo-3-azabicyclo[3.1.0]hexan-3- yl)pyridin-4-yl)phenyl)-3-(2,2,2- trifluoroethyl)pyrrolidine-1-carboxamide |
| 118 | 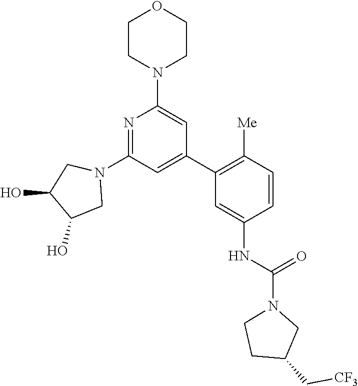 [SMILES: Cc1ccc(NC(=O)N2CC[C@@H](CC(F)(F)F)C2)cc1-c1cc(N2CCOCC2)nc(N2C[C@H](O)[C@@H](O)C2)c1] | (S)-N-(3-(2-((3S,4S)-3,4- dihydroxypyrrolidin-1-yl)-6- morpholinopyridin-4-yl)-4- methylphenyl)-3-(2,2,2- trifluoroethyl)pyrrolidine-1-carboxamide |
| 119 | 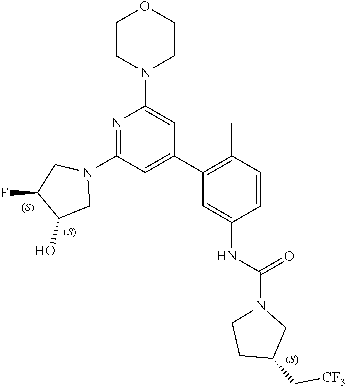 | (S)-N-(3-(2-((3S,4S)-3-fluoro-4- hydroxypyrrolidin-1-yl)-6- morpholinopyridin-4-yl)-4- methylphenyl)-3-(2,2,2- trifluoroethyl)pyrrolidine-1-carboxamide |
| 120 | 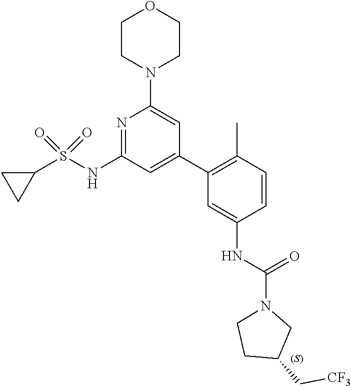 | (3S)-N-[3-[2-cyclopropanesulfonamido- 6-(morpholin-4-yl)pyridin-4-yl]-4- methylphenyl]-3-(2,2,2 trifluoroethyl)pyrrolidine-1-carboxamide |
| 121 | 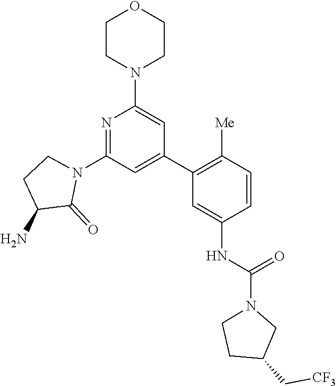 [SMILES: Cc1ccc(NC(=O)N2CC[C@@H](CC(F)(F)F)C2)cc1-c1cc(N2CCOCC2)nc(N2CC[C@H](N)C2=O)c1] | (S)-N-(3-(2-((S)-3-amino-2- oxopyrrolidin-1-yl)-6- morpholinopyridin-4-yl)-4- methylphenyl)-3-(2,2,2- trifluoroethyl)pyrrolidine-1-carboxamide |
| 122 | 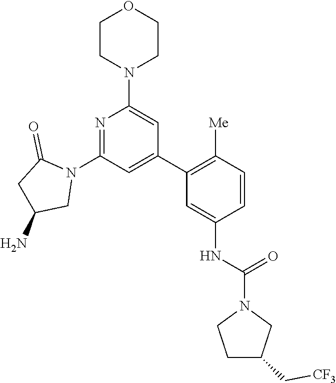 [SMILES: Cc1ccc(NC(=O)N2CC[C@@H](CC(F)(F)F)C2)cc1-c1cc(N2CCOCC2)nc(N2C[C@@H](N)CC2=O)c1] | (S)-N-(3-(2-((S)-4-amino-2- oxopyrrolidin-1-yl)-6- morpholinopyridin-4-yl)-4- methylphenyl)-3-(2,2,2- trifluoroethyl)pyrrolidine-1-carboxamide |
| 123 | 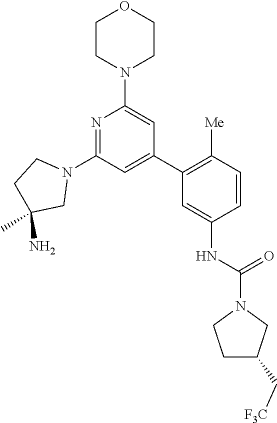 [SMILES: Cc1ccc(NC(=O)N2CC[C@@H](CC(F)(F)F)C2)cc1-c1cc(N2CCOCC2)nc(N2CC[C@](C)(N)C2)c1] | (S)-N-(3-(2-((S)-3-amino-3- methylpyrrolidin-1-yl)-6- morpholinopyridin-4-yl)-4- methylphenyl)-3-(2,2,2- trifluoroethyl)pyrrolidine-1-carboxamide |
| 124 | 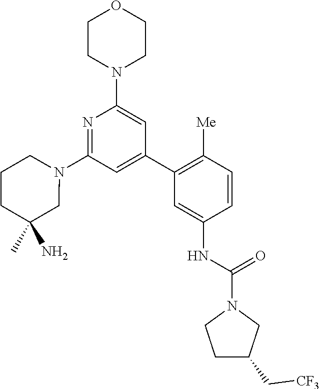 [SMILES: Cc1ccc(NC(=O)N2CC[C@@H](CC(F)(F)F)C2)cc1-c1cc(N2CCOCC2)nc(N2CCC[C@](C)(N)C2)c1] | (S)-N-(3-(2-((S)-3-amino-3- methylpiperidin-1-yl)-6- morpholinopyridin-4-yl)-4- methylphenyl)-3-(2,2,2- trifluoroethyl)pyrrolidine-1-carboxamide |
| 125 | 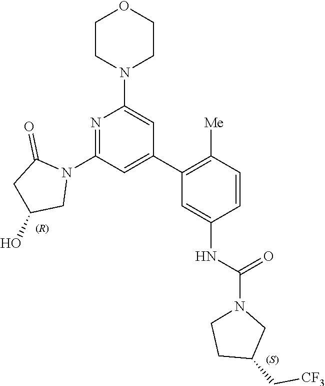 | (3S)-N-(3-[2-[(4R)-4-hydroxy-2- oxopyrrolidin-1-yl]-6-(morpholin-4- yl)pyridin-4-yl]-4-methylphenyl)-3- (2,2,2-trifluoroethyl)pyrrolidine-1- carboxamide |
| 126 | 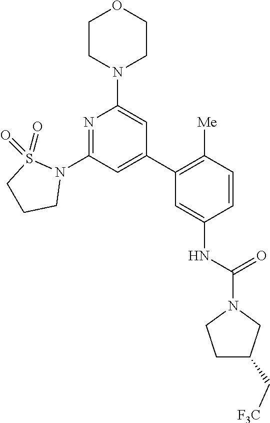 [SMILES: Cc1ccc(NC(=O)N2CC[C@@H](CC(F)(F)F)C2)cc1-c1cc(N2CCOCC2)nc(N2CCCS2(=O)=O)c1] | (3S)-N-[3-[2-(1,1-dioxo-1lambda6,2- thiazolidin-2-yl)-6-(morpholin-4- yl)pyridin-4-yl]-4-methylphenyl]-3- (2,2,2-trifluoroethyl)pyrrolidine-1- carboxamide |
| 127 | 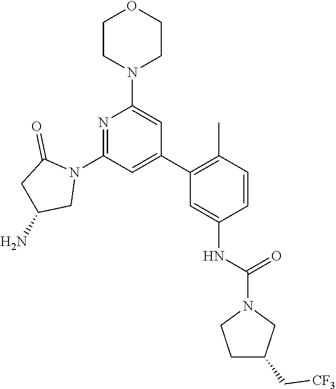 [SMILES: Cc1ccc(NC(=O)N2CC[C@@H](CC(F)(F)F)C2)cc1-c1cc(N2CCOCC2)nc(N2C[C@H](N)CC2=O)c1] | (S)-N-(3-(2-((R)-4-amino-2- oxopyrrolidin-1-yl)-6- morpholinopyridin-4-yl)-4- methylphenyl)-3-(2,2,2- trifluoroethyl)pyrrolidine-1-carboxamide |
| 128 and 129 | 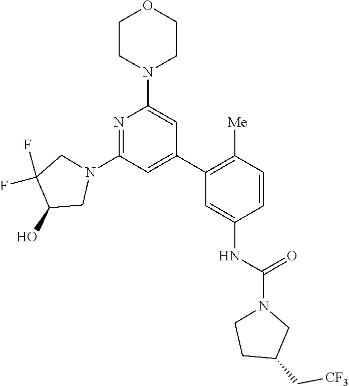 [SMILES: Cc1ccc(NC(=O)N2CC[C@@H](CC(F)(F)F)C2)cc1-c1cc(N2CCOCC2)nc(N2C[C@@H](O)C(F)(F)C2)c1] | (S)-N-(3-(2-((R)-3,3-difluoro-4- hydroxypyrrolidin-1-yl)-6- morpholinopyridin-4-yl)-4- methylphenyl)-3-(2,2,2- trifluoroethyl)pyrrolidine-1-carboxamide and (S)-N-(3-(2-((S)-3,3-difluoro-4- hydroxypyrrolidin-1-yl)-6- morpholinopyridin-4-yl)-4- methylphenyl)-3-(2,2,2- trifluoroethyl)pyrrolidine-1-carboxamide |
| | + | |
| | 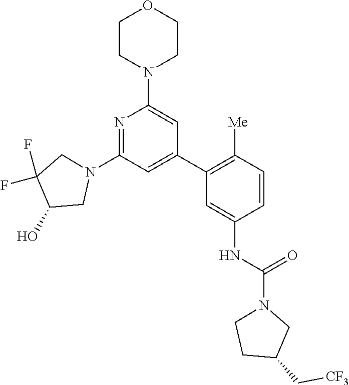 [SMILES: Cc1ccc(NC(=O)N2CC[C@@H](CC(F)(F)F)C2)cc1-c1cc(N2CCOCC2)nc(N2C[C@H](O)C(F)(F)C2)c1] | |
| 130 and 131 | 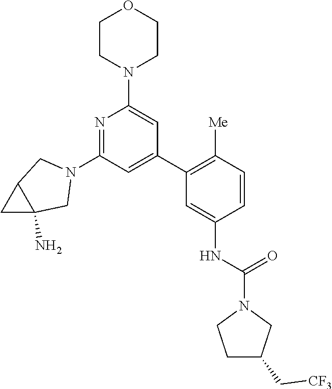 [SMILES: Cc1ccc(NC(=O)N2CC[C@@H](CC(F)(F)F)C2)cc1-c1cc(N2CCOCC2)nc(N2CC3C[C@]3(N)C2)c1] | (3S)-N-(3-(2-((1R)-1-amino-3- azabicyclo[3.1.0]hexan-3-yl)-6- morpholinopyridin-4-yl)-4- methylphenyl)-3-(2,2,2- trifluoroethyl)pyrrolidine-1-carboxamide and (3S)-N-(3-(2-((1S)-1-amino-3- azabicyclo[3.1.0]hexan-3-yl)-6- morpholinopyridin-4-yl)-4- methylphenyl)-3-(2,2,2- trifluoroethyl)pyrrolidine-1-carboxamide |
| | + | |
| | 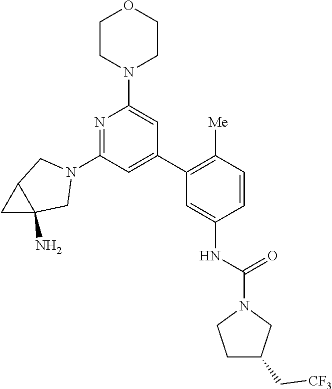 [SMILES: Cc1ccc(NC(=O)N2CC[C@@H](CC(F)(F)F)C2)cc1-c1cc(N2CCOCC2)nc(N2CC3C[C@@]3(N)C2)c1] | |
| 132 | 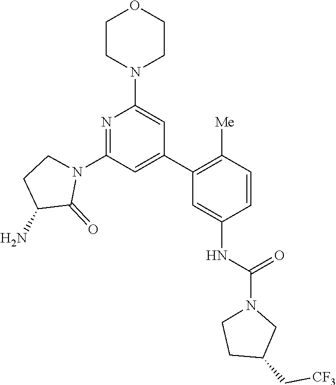 [SMILES: Cc1ccc(NC(=O)N2CC[C@@H](CC(F)(F)F)C2)cc1-c1cc(N2CCOCC2)nc(N2CC[C@@H](N)C2=O)c1] | (S)-N-(3-(2-((R)-4-amino-2- oxopyrrolidin-1-yl)-6- morpholinopyridin-4-yl)-4- methylphenyl)-3-(2,2,2- trifluoroethyl)pyrrolidine-1-carboxamide |
| 133 and 134 | 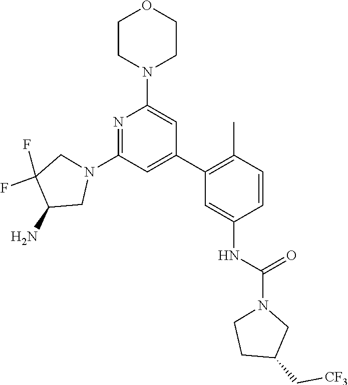 [SMILES: Cc1ccc(NC(=O)N2CC[C@@H](CC(F)(F)F)C2)cc1-c1cc(N2CCOCC2)nc(N2C[C@@H](N)C(F)(F)C2)c1] | (S)-N-(3-(2-((R)-4-amino-3,3- difluoropyrrolidin-1-yl)-6- morpholinopyridin-4-yl)-4- methylphenyl)-3-(2,2,2- trifluoroethyl)pyrrolidine-1-carboxamide and (S)-N-(3-(2-((S)-4-amino-3,3- difluoropyrrolidin-1-yl)-6- morpholinopyridin-4-yl)-4- methylphenyl)-3-(2,2,2- trifluoroethyl)pyrrolidine-1-carboxamide |
| | + | |
| | 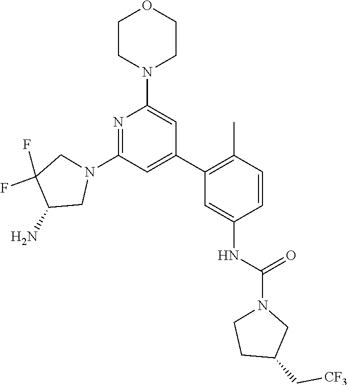 [SMILES: Cc1ccc(NC(=O)N2CC[C@@H](CC(F)(F)F)C2)cc1-c1cc(N2CCOCC2)nc(N2C[C@H](N)C(F)(F)C2)c1] | |
| 135 and 136 | 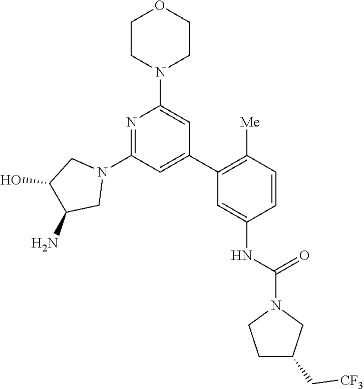 [SMILES: Cc1ccc(NC(=O)N2CC[C@@H](CC(F)(F)F)C2)cc1-c1cc(N2CCOCC2)nc(N2C[C@@H](N)[C@H](O)C2)c1] | (3S)-N-(3-{2-[(3R,4R)-3-amino-4- hydroxypyrrolidin-1-yl]-6-(morpholin-4- yl)pyridin-4-yl}-4-methylphenyl)-3- (2,2,2-trifluoroethyl)pyrrolidine-1- carboxamide and (3S)-N-(3-{2-[(3S,4S)- 3-amino-4-hydroxypyrrolidin-1-yl]-6- (morpholin-4-yl)pyridin-4-yl}-4- methylphenyl)-3-(2,2,2- trifluoroethyl)pyrrolidine-1-carboxamide |
| | + | |
| | 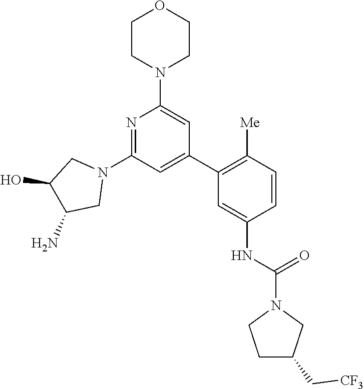 [SMILES: Cc1ccc(NC(=O)N2CC[C@@H](CC(F)(F)F)C2)cc1-c1cc(N2CCOCC2)nc(N2C[C@H](N)[C@@H](O)C2)c1] | |
| 137 | 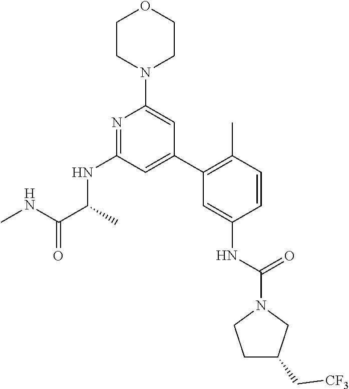 [SMILES: CNC(=O)[C@@H](C)Nc1cc(-c2cc(NC(=O)N3CC[C@@H](CC(F)(F)F)C3)ccc2C)cc(N2CCOCC2)n1] | (S)-N-(4-methyl-3-(2-(((R)-1- (methylamino)-1-oxopropan-2- yl)amino)-6-morpholinopyridin-4- yl)phenyl)-3-(2,2,2- trifluoroethyl)pyrrolidine-1-carboxamide |
| 138 | 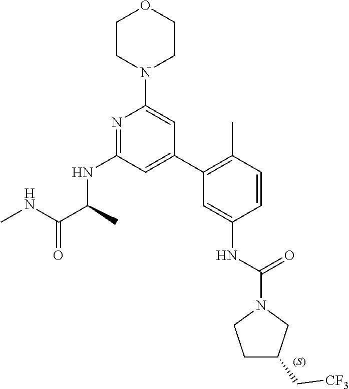 | (S)-N-(4-methyl-3-(2-(((S)-1- (methylamino)-1-oxopropan-2- yl)amino)-6-morpholinopyridin-4- yl)phenyl)-3-(2,2,2- trifluoroethyl)pyrrolidine-1-carboxamide |
| 139 | 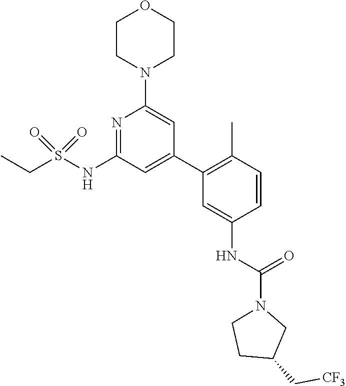 [SMILES: CCS(=O)(=O)Nc1cc(-c2cc(NC(=O)N3CC[C@@H](CC(F)(F)F)C3)ccc2C)cc(N2CCOCC2)n1] | (3S)-N-{3-[2-ethanesulfonamido-6- (morpholin-4-yl)pyridin-4-yl]-4- methylphenyl}-3-(2,2,2- trifluoroethyl)pyrrolidine-1-carboxamide |
| 140 | 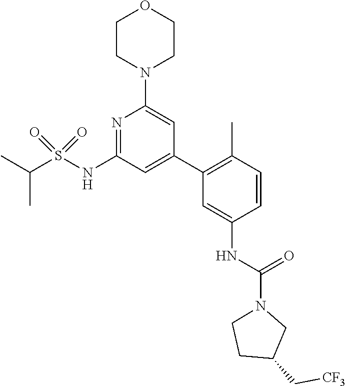 [SMILES: Cc1ccc(NC(=O)N2CC[C@@H](CC(F)(F)F)C2)cc1-c1cc(NS(=O)(=O)C(C)C)nc(N2CCOCC2)c1] | (3S)-N-{4-methyl-3-[2-(morpholin-4-yl)- 6-(propane-2-sulfonamido)pyridin-4- yl]phenyl}-3-(2,2,2- trifluoroethyl)pyrrolidine-1-carboxamide |
| 141 | 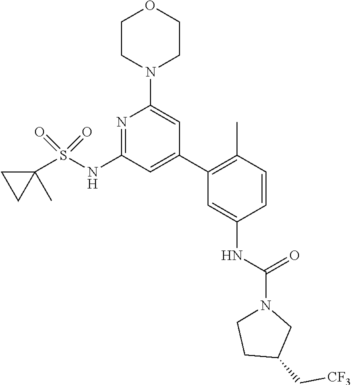 [SMILES: Cc1ccc(NC(=O)N2CC[C@@H](CC(F)(F)F)C2)cc1-c1cc(NS(=O)(=O)C2(C)CC2)nc(N2CCOCC2)c1] | (3S)-N-{4-methyl-3-[2-(1- methylcyclopropanesulfonamido)-6- (morpholin-4-yl)pyridin-4-yl]phenyl}-3- (2,2,2-trifluoroethyl)pyrrolidine-1- carboxamide |

### Preparation of Compounds

The compounds used in the reactions described herein are made according to organic synthesis techniques known to those skilled in this art, starting from commercially available chemicals and/or from compounds described in the chemical literature. “Commercially available chemicals” are obtained from standard commercial sources including Acros Organics (Pittsburgh, PA), Aldrich Chemical (Milwaukee, WI, including Sigma Chemical and Fluka), Apin Chemicals Ltd. (Milton Park, UK), Avocado Research (Lancashire, U.K.), BDH Inc. (Toronto, Canada), Bionet (Cornwall, U.K.), Chemservice Inc. (West Chester, PA), Crescent Chemical Co. (Hauppauge, NY), Eastman Organic Chemicals, Eastman Kodak Company (Rochester, NY), Fisher Scientific Co. (Pittsburgh, PA), Fisons Chemicals (Leicestershire, UK), Frontier Scientific (Logan, UT), ICN Biomedicals, Inc. (Costa Mesa, CA), Key Organics (Cornwall, U.K.), Lancaster Synthesis (Windham, NH), Maybridge Chemical Co. Ltd. (Cornwall, U.K.), Parish Chemical Co. (Orem, UT), Pfaltz & Bauer, Inc. (Waterbury, CN), Polyorganix (Houston, TX), Pierce Chemical Co. (Rockford, IL), Riedel de Haen AG (Hanover, Germany), Spectrum Quality Product, Inc. (New Brunswick, NJ), TCI America (Portland, OR), Trans World Chemicals, Inc. (Rockville, MD), and Wako Chemicals USA, Inc. (Richmond, VA).

Suitable reference books and treatise that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatise that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Specific and analogous reactants are optionally identified through the indices of known chemicals prepared by the Chemical Abstract Service of the American Chemical Society, which are available in most public and university libraries, as well as through on-line databases (contact the American Chemical Society, Washington, D.C. for more details). Chemicals that are known but not commercially available in catalogs are optionally prepared by custom chemical synthesis houses, where many of the standard chemical supply houses (e.g., those listed above) provide custom synthesis services. A reference useful for the preparation and selection of pharmaceutical salts of the compounds described herein is P. H. Stahl & C. G. Wermuth “Handbook of Pharmaceutical Salts”, Verlag Helvetica Chimica Acta, Zurich, 2002.

### Pharmaceutical Compositions

In certain embodiments, the heteroaromatic RAF kinase inhibitory compound described herein is administered as a pure chemical. In other embodiments, the heteroaromatic RAF kinase inhibitory compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, PA (2005)).

Provided herein is a pharmaceutical composition comprising at least one heteroaromatic RAF kinase inhibitory compound as described herein, or a stereoisomer, pharmaceutically acceptable salt, hydrate, or solvate thereof, together with one or more pharmaceutically acceptable carriers.

The carrier(s) (or excipient(s)) is acceptable or suitable if the carrier is compatible with the other ingredients of the composition and not deleterious to the recipient (i.e., the subject or the patient) of the composition.

One embodiment provides a pharmaceutical composition comprising a pharmaceutically acceptable excipient and a compound of Formula (I)-(IV), or a pharmaceutically acceptable salt or solvate thereof.

One embodiment provides a method of preparing a pharmaceutical composition comprising mixing a compound of Formula (I)-(IV), or a pharmaceutically acceptable salt or solvate thereof, and a pharmaceutically acceptable carrier.

In certain embodiments, the heteroaromatic RAF kinase inhibitory compound as described by Formula (I)-(IV), or a pharmaceutically acceptable salt or solvate thereof, is substantially pure, in that it contains less than about 5%, or less than about 1%, or less than about 0.1%, of other organic small molecules, such as unreacted intermediates or synthesis by-products that are created, for example, in one or more of the steps of a synthesis method.

Suitable oral dosage forms include, for example, tablets, pills, sachets, or capsules of hard or soft gelatin, methylcellulose or of another suitable material easily dissolved in the digestive tract. In some embodiments, suitable nontoxic solid carriers are used which include, for example, pharmaceutical grades of mannitol, lactose, starch, magnesium stearate, sodium saccharin, talcum, cellulose, glucose, sucrose, magnesium carbonate, and the like. (See, e.g., Remington: The Science and Practice of Pharmacy (Gennaro, 21^{st} Ed. Mack Pub. Co., Easton, PA (2005)).

In some embodiments, the heteroaromatic RAF kinase inhibitory compound as described by Formula (I)-(IV), or pharmaceutically acceptable salt or solvate thereof, is formulated for administration by injection. In some instances, the injection formulation is an aqueous formulation. In some instances, the injection formulation is a non-aqueous formulation. In some instances, the injection formulation is an oil-based formulation, such as sesame oil, or the like.

The dose of the composition comprising at least one heteroaromatic RAF kinase inhibitory compound as described herein differs depending upon the subject or patient's (e.g., human) condition. In some embodiments, such factors include general health status, age, and other factors.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. The optimal dose depends upon the body mass, weight, or blood volume of the patient.

Oral doses typically range from about 1.0 mg to about 1000 mg, one to four times, or more, per day.

### Methods of Treatment

One embodiment provides a compound of Formula (I)-(IV), or a pharmaceutically acceptable salt or solvate thereof, for use in a method of treatment of the human or animal body.

One embodiment provides a compound of Formula (I)-(IV), or a pharmaceutically acceptable salt or solvate thereof, for use in a method of treatment of cancer or neoplastic disease.

One embodiment provides a use of a compound of Formula (I)-(IV), or a pharmaceutically acceptable salt or solvate thereof, in the manufacture of a medicament for the treatment of cancer or neoplastic disease.

In some embodiments, described herein is a method of treating cancer in a patient in need thereof comprising administering to the patient a compound of Formula (I)-(IV), or a pharmaceutically acceptable salt or solvate thereof. In some embodiments, described herein is a method of treating cancer in a patient in need thereof comprising administering to the patient a pharmaceutical composition comprising a compound of Formula (I)-(IV), or a pharmaceutically acceptable salt or solvate thereof, and a pharmaceutically acceptable excipient.

Provided herein is the method wherein the pharmaceutical composition is administered orally. Provided herein is the method wherein the pharmaceutical composition is administered by injection.

Other embodiments and uses will be apparent to one skilled in the art in light of the present disclosures. The following examples are provided merely as illustrative of various embodiments and shall not be construed to limit the invention in any way.

### Examples

### I. Chemical Synthesis

In some embodiments, the heteroaromatic RAF kinase inhibitory compounds disclosed herein are synthesized according to the following examples. As used below, and throughout the description of the invention, the following abbreviations, unless otherwise indicated, shall be understood to have the following meanings:
° C. degrees Celsius
δ_{H} chemical shift in parts per million downfield from tetramethylsilane
DCM dichloromethane (CH₂Cl₂)
DMF dimethylformamide
DMSO dimethylsulfoxide
EA ethyl acetate
ESI electrospray ionization
Et ethyl
g gram(s)
h hour(s)
HPLC high performance liquid chromatography
Hz hertz
J coupling constant (in NMR spectrometry)
LCMS liquid chromatography mass spectrometry
μ micro
m multiplet (spectral); meter(s); milli
M molar
M⁺ parent molecular ion
Me methyl
MHz megahertz
min minute(s)
mol mole(s); molecular (as in mol wt)
mL milliliter
MS mass spectrometry
nm nanometer(s)
NMR nuclear magnetic resonance
pH potential of hydrogen; a measure of the acidity or basicity of an aqueous
solution
PE petroleum ether
RT room temperature
s singlet (spectral)
t triplet (spectral)
T temperature
TFA trifluoroacetic acid
THF tetrahydrofuran

### Example 1: (3S)-N-(3-[6-[(3,3-difluorocyclopentyl)amino]-2-(morpholin-4-yl)pyrimidin-4-yl]-4-methylphenyl)-3-(2,2,2-trifluoroethyl)pyrrolidine-1-carboxamide

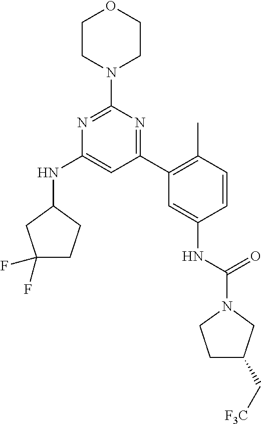 [SMILES: Cc1ccc(NC(=O)N2CC[C@@H](CC(F)(F)F)C2)cc1-c1cc(NC2CCC(F)(F)C2)nc(N2CCOCC2)n1]

### Synthetic Scheme

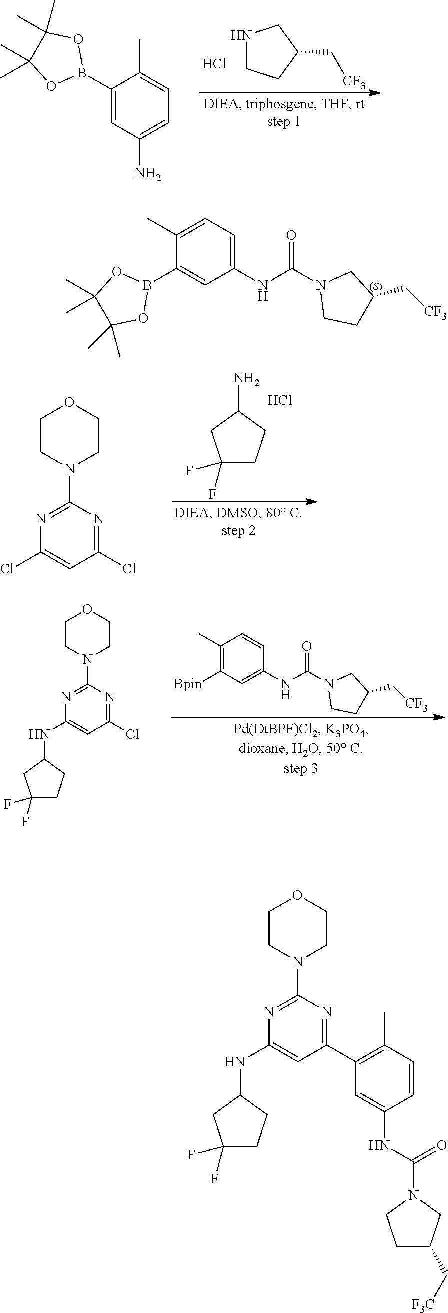

Preparation 1A: (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide

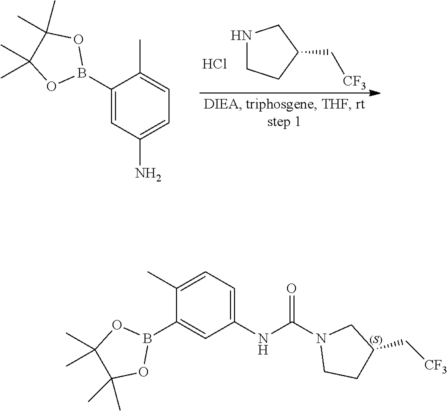

To a stirred solution of 4-methyl-3-(4,4,5,5-tetramethyl-1,3,2-dioxaborolan-2-yl)aniline (11.0 g, 47.186 mmol, 1.00 equiv) in THF (1000 mL) were added DIEA (30.5 g, 235.930 mmol, 5.00 equiv) and triphosgene (5.6 g, 18.874 mmol, 0.40 equiv) at ambient temperature under nitrogen atmosphere. The resulting mixture was stirred at ambient temperature for 30 min. To this was added a solution of (3S)-3-(2,2,2-trifluoroethyl)pyrrolidine hydrochloride (8.95 g, 47.186 mmol, 1.00 equiv) in THF (100 mL). The resulting mixture was stirred at ambient temperature for 3 h. The reaction mixture was concentrated under reduced pressure. The residue was purified by silica gel column chromatography, eluted with EtOAc in PE (5%-45%) to afford (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (16.3 g, 84%) as a white solid. MS ESI calculated for C₂₀H₂₈BF₃N₂O₃ [M+H]⁺, 413.21, found 413.25. ¹H NMR (300 MHz, chloroform-d) δ 7.75 (dd, J=8.4, 2.7 Hz, 1H), 7.44 (d, J=2.7 Hz, 1H), 7.14 (d, J=8.4 Hz, 1H), 6.14 (s, 1H), 3.86-3.74 (m, 1H), 3.69-3.56 (m, 1H), 3.43 (td, J=9.6, 6.6 Hz, 1H), 3.11 (t, J=9.6 Hz, 1H), 2.58 (dd, J=16.5, 8.4 Hz, 1H), 2.50 (s, 3H), 2.37-2.18 (m, 3H), 1.82-1.75 (m, 1H), 1.36 (s, 12H). ¹⁹F NMR (282 MHz, chloroform-d) δ-64.95.

Preparation 1B: N-(3,3-dimethylcyclopentyl)-6-methyl-2-(morpholin-4-yl)pyrimidin-4-amine

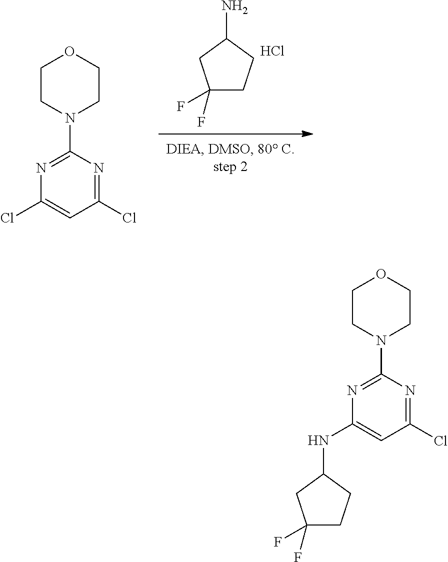 [SMILES: Clc1cc(Cl)nc(N2CCOCC2)n1 ; NC1CCC(F)(F)C1 ; Cl ; FC1(F)CCC(Nc2cc(Cl)nc(N3CCOCC3)n2)C1]

To a stirred solution of 4-(4,6-dichloropyrimidin-2-yl)morpholine (140 mg, 0.598 mmol) and 3,3-difluorocyclopentan-1-amine hydrochloride (104 mg, 0.658 mmol) in DMSO (1 mL) was added DIEA (386 mg, 2.990 mmol) dropwise at 0° C. under nitrogen atmosphere. The resulting mixture was stirred for 16 at room temperature under nitrogen atmosphere. The reaction was quenched by the addition of water (100 mL). The resulting mixture was extracted with EtOAc (3×50 mL). The combined organic layers were washed with saturated brine (3×50 mL), dried over anhydrous Na₂SO₄. After filtration, the filtrate was concentrated under reduced pressure. The residue was purified by silica gel column chromatography, eluted with PE/(EA/EtOH=3:1) (3:1) to afford N-(3,3-dimethylcyclopentyl)-6-methyl-2-(morpholin-4-yl)pyrimidin-4-amine (160 mg, 92%) as an off-white solid. MS ESI calculated for C₁₃H₁₇ClF₂N₄O [M+H]⁺, 319.11, found 319.15. ¹H NMR (400 MHz, DMSO-d₆) δ 7.55 (s, 1H), 5.81 (s, 1H), 4.36 (s, 1H), 3.61 (s, 8H), 3.42 (s, 2H), 2.62-2.55 (m, 1H), 2.28-1.92 (m, 2H), 1.73-1.63 (m, 1H). ¹⁹F NMR (376 MHz, DMSO-d₆) δ-87.90 (2F).

### Example 1: (3S)-N-(3-[6-[(3,3-difluorocyclopentyl)amino]-2-(morpholin-4-yl)pyrimidin-4-yl]-4-methylphenyl)-3-(2,2,2-trifluoroethyl)pyrrolidine-1-carboxamide

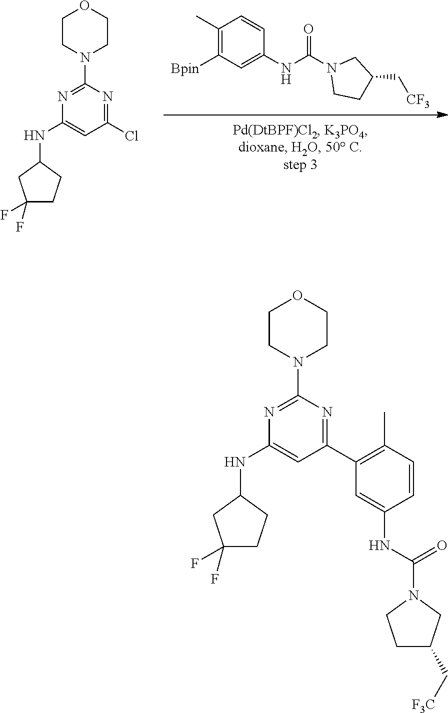

To a stirred solution of 6-chloro-N-(3,3-difluorocyclopentyl)-2-(morpholin-4-yl)pyrimidin-4-amine (140 mg, 0.439 mmol), (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (181 mg, 0.439 mmol) and K₃PO₄ (186 mg, 0.878 mmol,) in dioxane:H₂O=2:1 (2 mL) was added Pd(DtBPF)Cl₂ (29 mg, 0.044 mmol) at room temperature under nitrogen atmosphere. The resulting mixture was stirred for 16 h at 60° C. under nitrogen atmosphere. The reaction was monitored by LCMS. The reaction was quenched by the addition of water (100 mL). The resulting mixture was extracted with EtOAc (3×50 mL). The combined organic layers were washed with saturated brine (3×50 mL), dried over anhydrous Na₂SO₄. After filtration, the filtrate was concentrated under reduced pressure. The residue was purified by silica gel column chromatography, eluted with PE/(EA/EtOH=3:1) (2:1) to afford 150 mg crude product. The residue was purified by reverse flash chromatography with the following conditions: Column: XBridge C18 OBD Prep Column, 100Å, 10 μm, 19 mm×250 mm; Mobile Phase A: Water (10 MMOL/L NH₄HCO₃), Mobile Phase B: CH₃CN; Flow rate:20 mL/min; Gradient:40% B to 80% B in 5.8 min; 210/254 nm to afford (3S)-N-(3-[6-[(3,3-difluorocyclopentyl)amino]-2-(morpholin-4-yl)pyrimidin-4-yl]-4-methylphenyl)-3-(2,2,2-trifluoroethyl)pyrrolidine-1-carboxamide (130 mg, 52%) as an off-white solid. MS ESI calculated for C27H₃₃F₅N₆O₂ [M+H]⁺, 569.26, found 569.25. ¹H NMR (400 MHz, DMSO-d₆) δ 8.13 (s, 1H), 7.52 (m, 1H), 7.42-7.41 (m, 1H), 7.30-7.28 (m, 1H), 7.09-7.06 (m, 1H), 5.86 (s, 1H), 4.40 (s, 1H), 3.69-3.64 (m, 9H), 3.55-3.50 (m, 1H), 3.31-3.28 (m, 1H), 3.05-3.00 (m, 1H), 2.59-2.51 (m, 1H), 2.49-2.40 (m, 3H), 2.26-2.13 (m, 5H), 2.13-2.03 (m, 3H), 1.75-1.66 (m, 2H). ¹⁹F NMR (376 MHz, DMSO-d₆) δ-63.36 (3F),−87.70-87.80 (2F).

### Examples 2 and 3: (3S)-N-[3-(6-[[(1R)-3,3-difluorocyclopentyl]amino]-2-(morpholin-4-yl)pyrimidin-4-yl)-4-methylphenyl]-3-(2,2,2-trifluoroethyl)pyrrolidine-1-carboxamide, (3S)-N-[3-(6-[[(1S)-3,3-difluorocyclopentyl]amino]-2-(morpholin-4-yl)pyrimidin-4-yl)-4-methylphenyl]-3-(2,2,2-trifluoroethyl)pyrrolidine-1-carboxamide

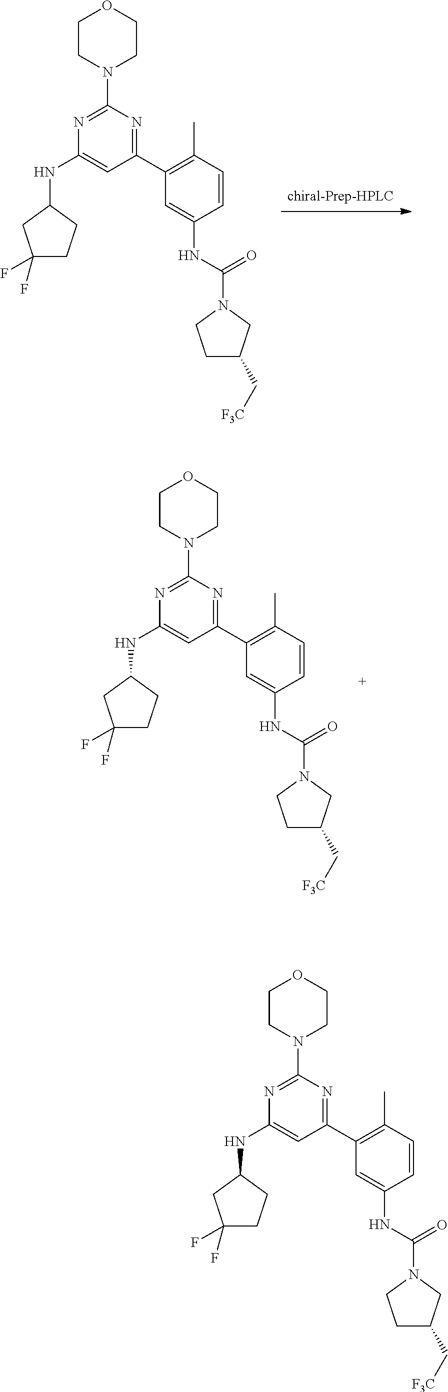 [SMILES: Cc1ccc(NC(=O)N2CC[C@@H](CC(F)(F)F)C2)cc1-c1cc(NC2CCC(F)(F)C2)nc(N2CCOCC2)n1 ; Cc1ccc(NC(=O)N2CC[C@@H](CC(F)(F)F)C2)cc1-c1cc(N[C@@H]2CCC(F)(F)C2)nc(N2CCOCC2)n1 ; Cc1ccc(NC(=O)N2CC[C@@H](CC(F)(F)F)C2)cc1-c1cc(N[C@H]2CCC(F)(F)C2)nc(N2CCOCC2)n1]

(3S)-N-(3-[6-[(3,3-difluorocyclopentyl)amino]-2-(morpholin-4-yl)pyrimidin-4-yl]-4-methylphenyl)-3-(2,2,2-trifluoroethyl)pyrrolidine-1-carboxamide (130 mg) was purified by Chiral-Prep-HPLC with the following conditions (Column: CHIRALPAK AD-H, 2.0 cm*25 cm; Mobile Phase A:Hex (8 mmol/L NH₃·MeOH), Mobile Phase B: EtOH; Flow rate:20 mL/min; Gradient:20 B to 20 B in 18 min; 220/254 nm) to afford (3S)-N-[3-(6-[[(1R)-3,3-difluorocyclopentyl]amino]-2-(morpholin-4-yl)pyrimidin-4-yl)-4-methylphenyl]-3-(2,2,2-trifluoroethyl)pyrrolidine-1-carboxamide (41 mg, 32%) as an off-white solid, MS ESI calculated for C₂₇H₃₃F₅N₆O₂ [M+H]⁺, 569.26, found 569.25. ¹H NMR (400 MHz, DMSO-d₆) δ 8.14 (s, 1H), 7.52 (m, 1H), 7.43-7.41 (m, 1H), 7.30 (s, 1H), 7.09-7.07 (m, 1H), 5.86 (s, 1H), 4.40 (s, 1H), 3.69-3.64 (m, 9H), 3.55-3.50 (m, 1H), 3.31-3.28 (m, 1H), 3.05-3.00 (m, 1H), 2.61-2.55 (m, 1H), 2.46-2.38 (m, 3H), 2.26-2.13 (m, 5H), 2.13-1.91 (m, 3H), 1.78-1.61 (m, 2H). ¹⁹F NMR (376 MHz, DMSO-d₆) δ-63.33 (3F),−87.67 (2F).

Also afforded (3S)-N-[3-(6-[[(1S)-3,3-difluorocyclopentyl]amino]-2-(morpholin-4-yl)pyrimidin-4-yl)-4-methylphenyl]-3-(2,2,2-trifluoroethyl)pyrrolidine-1-carboxamide (44 mg, 34%) as an off-white solid. MS ESI calculated for C₂₇H₃₃F₅N₆O₂ [M+H]⁺, 569.26, found 569.25. ¹H NMR (400 MHz, DMSO-d₆) δ 8.14 (s, 1H), 7.52 (m, 1H), 7.43-7.40 (m, 1H), 7.30 (s, 1H), 7.09-7.06 (m, 1H), 5.86 (s, 1H), 4.40 (s, 1H), 3.69-3.64 (m, 9H), 3.55-3.50 (m, 1H), 3.31-3.28 (m, 1H), 3.05-3.00 (m, 1H), 2.61-2.55 (m, 1H), 2.46-2.38 (m, 3H), 2.26-2.13 (m, 5H), 2.13-1.91 (m, 3H), 1.78-1.61 (m, 2H). ¹⁹F NMR (376 MHz, DMSO-d₆) δ-63.36 (3F),−87.68 (2F).

### Examples 4 and 5: (3S)-N-[3-(2-[[(1S,3S)-3-hydroxycyclopentyl]amino]-6-(morpholin-4-yl)pyrimidin-4-yl)-4-methylphenyl]-3-(2,2,2-trifluoroethyl)pyrrolidine-1-carboxamide, (3S)-N-[3-(2-[[(1R,3R)-3-hydroxycyclopentyl]amino]-6-(morpholin-4-yl)pyrimidin-4-yl)-4-methylphenyl]-3-(2,2,2-trifluoroethyl)pyrrolidine-1-carboxamide

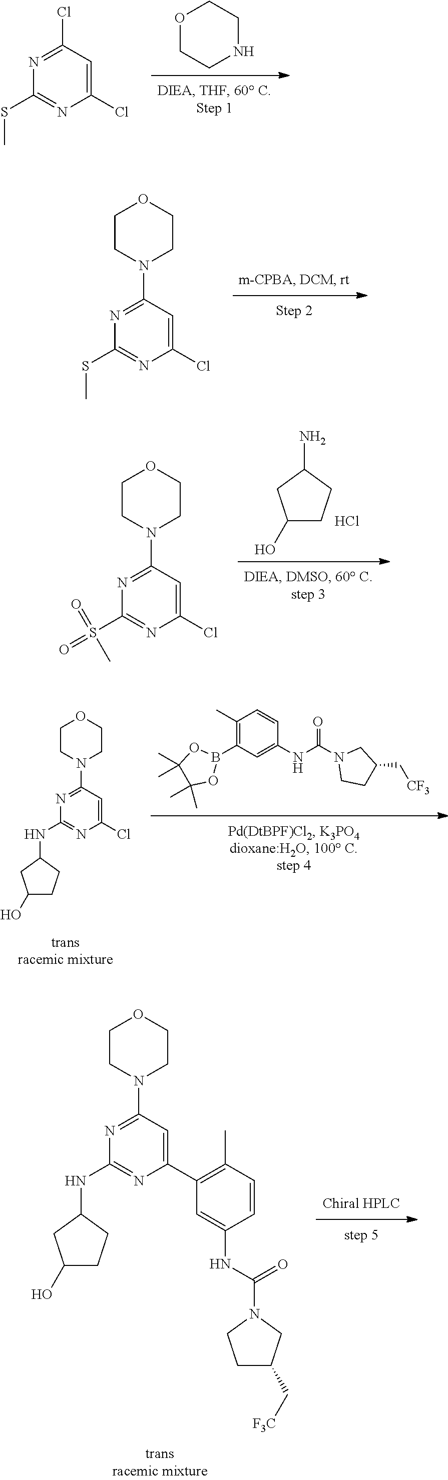

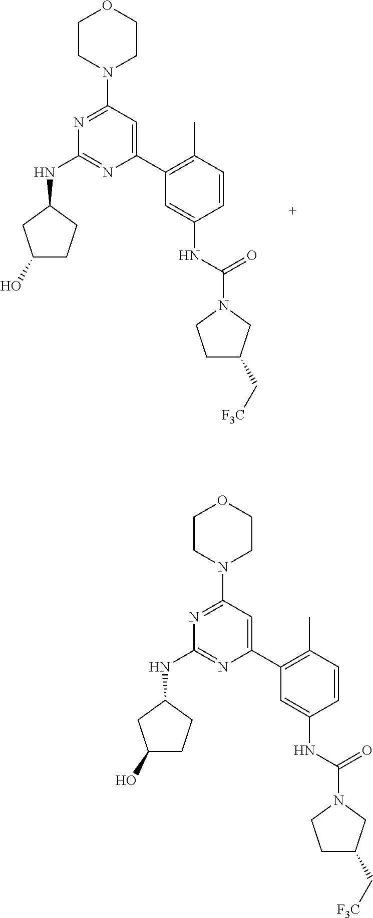 [SMILES: Cc1ccc(NC(=O)N2CC[C@@H](CC(F)(F)F)C2)cc1-c1cc(N2CCOCC2)nc(N[C@H]2CC[C@H](O)C2)n1 ; Cc1ccc(NC(=O)N2CC[C@@H](CC(F)(F)F)C2)cc1-c1cc(N2CCOCC2)nc(N[C@@H]2CC[C@@H](O)C2)n1]

Preparation 4A: 4-[6-chloro-2-(methylsulfanyl)pyrimidin-4-yl]morpholine

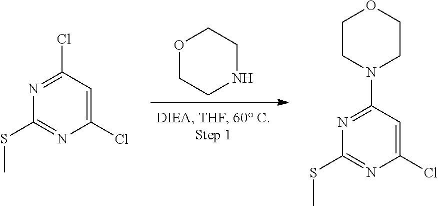

To a stirred solution of 4,6-dichloro-2-(methylsulfanyl)pyrimidine (2.20 g, 11.28 mmol) in THF (29 mL) was added DIPEA (1.60 g, 12.41 mmol), morpholine (1.08 g, 12.40 mmol) dropwise at room temperature under nitrogen atmosphere. The resulting mixture was stirred for overnight at 60° C. under nitrogen atmosphere. The mixture was allowed to cool down to room temperature. The resulting mixture was diluted with water (50 mL). The precipitated solids were collected by filtration to afford 4-[6-chloro-2-(methylsulfanyl)pyrimidin-4-yl]morpholine (2.7 g, 97%) as a light yellow solid. MS ESI calculated for C9H₁₂ClN₃OS [M+H]⁺, 246.04; found 245.95. ¹H NMR (400 MHz, DMSO-d₆) δ 6.68 (s, 1H), 3.68-3.64 (m, 8H), 2.45 (s, 3H).

Preparation 4B: 4-(6-chloro-2-methanesulfonylpyrimidin-4-yl)morpholine

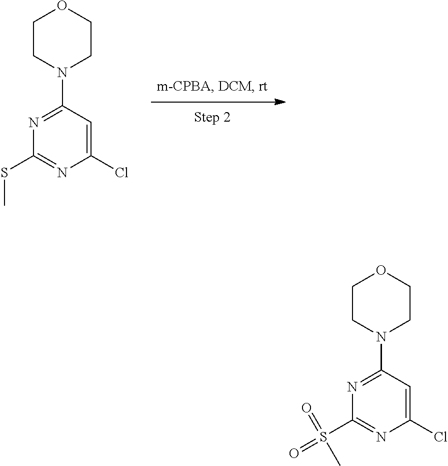 [SMILES: CSc1nc(Cl)cc(N2CCOCC2)n1 ; CS(=O)(=O)c1nc(Cl)cc(N2CCOCC2)n1]

To a stirred solution of 4-[6-chloro-2-(methylsulfanyl)pyrimidin-4-yl]morpholine (12.33 g, 50.18 mmol) and m-CPBA (23.43 g, 115.41 mmol, 85%) in DCM (172.6 mL) at room temperature under nitrogen atmosphere. The resulting mixture was stirred for 2 h at room temperature under nitrogen atmosphere. The resulting mixture was concentrated under reduced pressure. The reaction was quenched by the addition of NaHCO₃ aq. (200 mL) at 0° C. The resulting mixture was extracted with EtOAc (3×150 mL). The combined organic layers were washed with NaHCO₃ aq. (200 mL), dried over anhydrous Na₂SO₄. After filtration, the filtrate was concentrated under reduced pressure. The residue was purified by silica gel column chromatography, eluted with hexane/EtOAc (1:1) to afford 4-(6-chloro-2-methanesulfonylpyrimidin-4-yl)morpholine (13.3 g, 95%) as a white solid. MS ESI calculated for C9H₁₂ClN₃O₃S [M+H]⁺, 278.03; found 277.95. ¹H NMR (300 MHz, chloroform-d) δ 6.62 (s, 1H), 3.82-3.75 (m, 8H), 3.36-3.31 (m, 3H).

Preparation 4C: trans-3-[[4-chloro-6-(morpholin-4-yl)pyrimidin-2-yl]amino]cyclopentan-1-ol

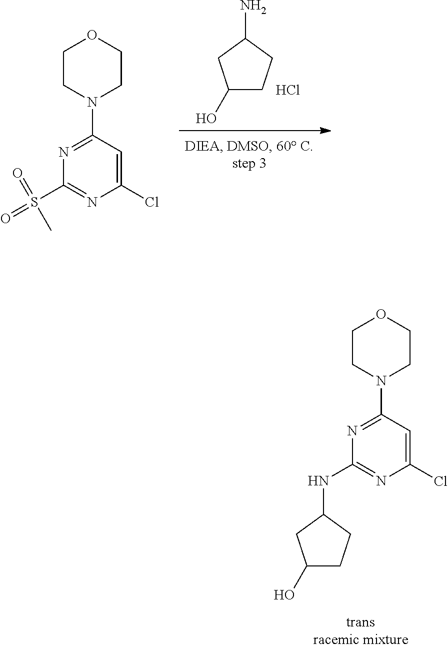 [SMILES: CS(=O)(=O)c1nc(Cl)cc(N2CCOCC2)n1 ; NC1CCC(O)C1 ; Cl ; OC1CCC(Nc2nc(Cl)cc(N3CCOCC3)n2)C1]

A mixture of 4-(6-chloro-2-methanesulfonylpyrimidin-4-yl)morpholine (1.00 g, 3.60 mmol), (trans)-3-aminocyclopentan-1-ol hydrochloride (0.55 g, 4.00 mmol) and DIEA (1.40 g, 10.80 mmol) in DMSO (10 mL) was stirred for overnight at 60° C. under nitrogen atmosphere. The resulting mixture was diluted with water (10 mL). The resulting mixture was extracted with EtOAc (3×10 mL). The combined organic layers were washed with brine (10 mL), dried over anhydrous Na₂SO₄. After filtration, the filtrate was concentrated under reduced pressure. The residue was purified by silica gel column chromatography, eluted with PE/(EtOAc: EtOH=3:1) (1:1) to afford trans-3-[[4-chloro-6-(morpholin-4-yl)pyrimidin-2-yl]amino]cyclopentan-1-ol (0.38 g, 33%) as an off-white solid. MS ESI calculated for C₁₃H₁₉ClN₄O₂[M+H]⁺, 299.77, found 299.10. ¹H NMR (300 MHz, DMSO-d₆) δ 6.07 (s, 1H), 4.29 (m, 1H), 4.18 (m, 1H), 3.64 (m, 4H), 3.54 (m, 4H), 3.40 (m, 2H), 2.05 (m, 1H), 1.86 (m, 2H), 1.64-1.58 (m, 1H), 1.41 (m, 2H).

Preparation 4D: (3S)-N-[3-(2-[[trans-3-hydroxycyclopentyl]amino]-6-(morpholin-4-yl)pyrimidin-4-yl)-4-methylphenyl]-3-(2,2,2-trifluoroethyl)pyrrolidine-1-carboxamide

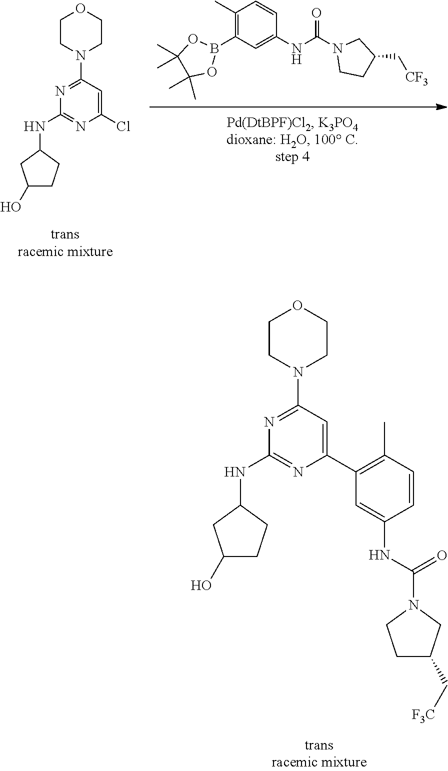 [SMILES: OC1CCC(Nc2nc(Cl)cc(N3CCOCC3)n2)C1 ; Cc1ccc(NC(=O)N2CC[C@@H](CC(F)(F)F)C2)cc1B1OC(C)(C)C(C)(C)O1 ; Cc1ccc(NC(=O)N2CC[C@@H](CC(F)(F)F)C2)cc1-c1cc(N2CCOCC2)nc(NC2CCC(O)C2)n1]

A mixture of trans-3-[[4-chloro-6-(morpholin-4-yl)pyrimidin-2-yl]amino]cyclopentan-1-ol (200 mg, 0.669 mmol), (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (276 mg, 0.669 mmol), Pd(DtBPF)Cl₂ (44 mg, 0.067 mmol) and K₃PO₄ (284 mg, 1.339 mmol) in 1,4-dioxane (3.6 mL) and H₂O (0.9 mL) was stirred for overnight at 80° C. under nitrogen atmosphere. The resulting mixture was concentrated under vacuum. The residue was purified by silica gel column chromatography, eluted with PE/((EtOAc:EtOH=3:1):NH₄OH=20:1) (1:1). The crude product was purified by reverse flash chromatography with the following conditions: Column: XBridge C¹⁸ OBD Prep Column, 100Å, 10 μm, 19 mm×250 mm; Mobile Phase A: water (10 mmol/L NH₄HCO₃), Mobile Phase B: CH₃CN; Flow rate:20 mL/min; Gradient:35 B to 70 B in 5.8 min; 210/254 nm to afford (3S)-N-[3-(2-[[(trans)-3-hydroxycyclopentyl]amino]-6-(morpholin-4-yl)pyrimidin-4-yl)-4-methylphenyl]-3-(2,2,2-trifluoroethyl)pyrrolidine-1-carboxamide (180 mg, 29%) as an off-white solid. MS ESI calculated for C₂₇H₃₅F₃N₆O₃[M+H]⁺, 549.27; found 549.30.

### Example 4: (3S)-N-[3-(2-[[(1S,3S)-3-hydroxycyclopentyl]amino]-6-(morpholin-4-yl)pyrimidin-4-yl)-4-methylphenyl]-3-(2,2,2-trifluoroethyl)pyrrolidine-1-carboxamide

### Examples 4 and 5: (3S)-N-[3-(2-[[(1R,3R)-3-hydroxycyclopentyl]amino]-6-(morpholin-4-yl)pyrimidin-4-yl)-4-methylphenyl]-3-(2,2,2-trifluoroethyl)pyrrolidine-1-carboxamide

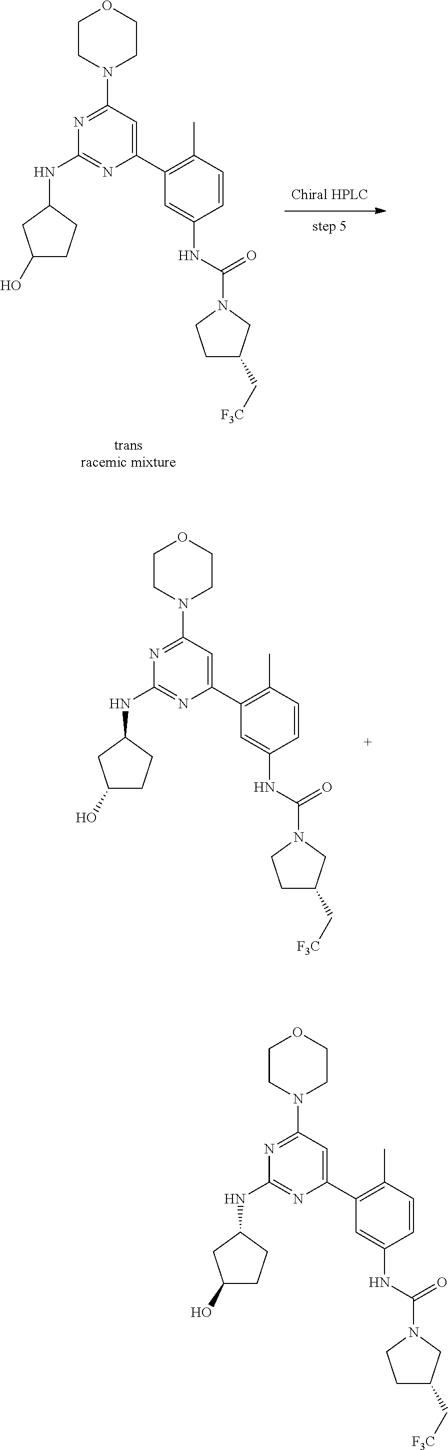 [SMILES: Cc1ccc(NC(=O)N2CC[C@@H](CC(F)(F)F)C2)cc1-c1cc(N2CCOCC2)nc(NC2CCC(O)C2)n1 ; Cc1ccc(NC(=O)N2CC[C@@H](CC(F)(F)F)C2)cc1-c1cc(N2CCOCC2)nc(N[C@H]2CC[C@H](O)C2)n1 ; Cc1ccc(NC(=O)N2CC[C@@H](CC(F)(F)F)C2)cc1-c1cc(N2CCOCC2)nc(N[C@@H]2CC[C@@H](O)C2)n1]

(3S)-N-[3-(2-[[(trans)-3-hydroxycyclopentyl]amino]-6-(morpholin-4-yl)pyrimidin-4-yl)-4-methylphenyl]-3-(2,2,2-trifluoroethyl)pyrrolidine-1-carboxamide (180 mg) was resolved by Chiral-HPLC with the following conditions (Column: Reg-AD, 30×250 mm, 5 um; Mobile Phase A: Hex (8 mmol/L NH₃·MeOH), Mobile Phase B: IPA; Flow rate: 45 mL/min; Gradient: 20 B to 20 B in 25 min; 220/254 nm) to afford 75 mg (44%) as an off-white solid. MS ESI calculated for C₂₇H₃₅F₃N₆O₃[M+H]⁺, 549.61; found 549.30. ¹H NMR (300 MHz, DMSO-d₆) δ 8.11 (s, 1H), 7.46-7.43 (m, 2H), 7.09-7.06 (m, 1H), 6.52-6.50 (m, 1H), 5.99 (s, 1H), 4.41-4.36 (m, 2H), 4.18-4.17 (m, 1H), 3.70-3.43 (m, 10H), 3.35-3.28 (m, 1H), 3.05-2.99 (m, 1H), 2.50-2.32 (m, 3H), 2.25-2.16 (m, 3H), 2.08-1.91 (m, 2H), 1.90-1.75 (m, 2H), 1.74-1.65 (m, 2H), 1.64-1.30 (m, 2H). ¹⁹F NMR (282 MHz, DMSO-d₆) δ-63.39-63.45 (3F), and (3S)-N-[3-(2-[[(1R,3R)-3-hydroxycyclopentyl]amino]-6-(morpholin-4-yl)pyrimidin-4-yl)-4-methylphenyl]-3-(2,2,2-trifluoroethyl)pyrrolidine-1-carboxamide (77 mg, 45%) as an off-white solid.MS ESI calculated for C₂₇H₃₅F₃N₆O₃[M+H]⁺, 549.61; found 549.30. ¹H NMR (300 MHz, DMSO-d₆) δ 8.11 (s, 1H), 7.46-7.43 (m, 2H), 7.09-7.06 (m, 1H), 6.52 (m, 1H), 5.99 (s, 1H), 4.41-4.32 (m, 2H), 4.17 (m, 1H), 3.69-3.64 (m, 5H), 3.55-3.50 (m, 5H), 3.35-3.26 (m, 1H), 3.05-2.99 (m, 1H), 2.51-2.40 (m, 3H), 2.25 (s, 3H), 2.07-1.99 (m, 2H), 1.93-1.85 (m, 2H), 1.70-1.55 (m, 2H), 1.54-1.25 (m, 2H). ¹⁹F NMR (376 MHz, DMSO-d₆) δ-63.35 (3F).

### Example 6: (3S)-N-[3-(2-[[(3-hydroxyoxetan-3-yl)methyl]amino]-6-(morpholin-4-yl)pyridin-4-yl)-4-methylphenyl]-3-(2,2,2-trifluoroethyl)pyrrolidine-1-carboxamide

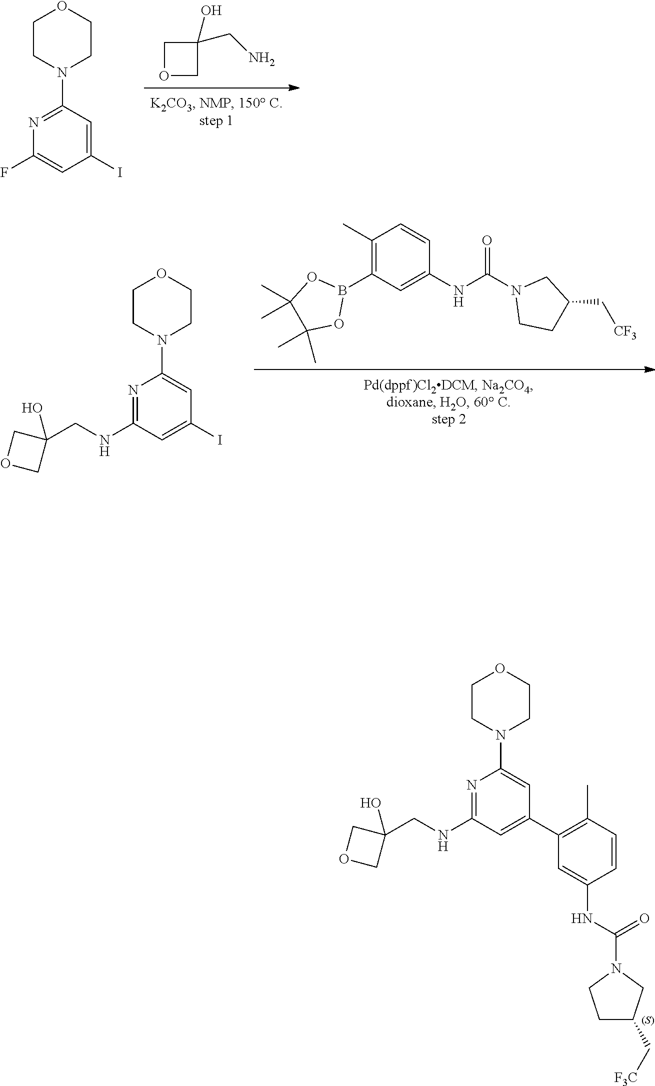

Preparation 6A: 3-([[4-iodo-6-(morpholin-4-yl)pyridin-2-yl]amino]methyl)oxetan-3-ol

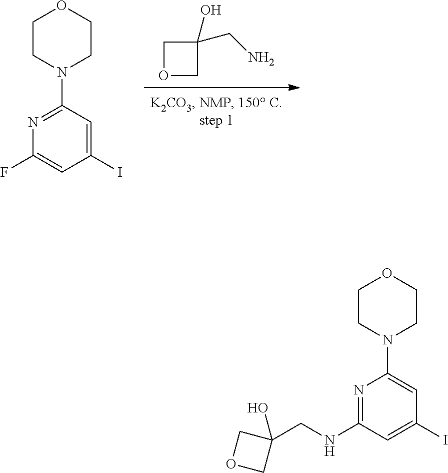 [SMILES: Fc1cc(I)cc(N2CCOCC2)n1 ; NCC1(O)COC1 ; OC1(CNc2cc(I)cc(N3CCOCC3)n2)COC1]

A mixture of 4-(6-fluoro-4-iodopyridin-2-yl)morpholine (500 mg, 1.623 mmol), 3-(aminomethyl)oxetan-3-ol (251 mg, 2.434 mmol) and K₂CO₃ (449 mg, 3.246 mmol) in NMP (15 mL) was stirred for overnight at 150° C. under argon atmosphere. The resulting mixture was concentrated under reduced pressure. The residue was purified by silica gel column chromatography, eluted with DCM/MeOH (20/1) to afford crude product. The residue was purified by reverse flash chromatography with the following conditions: column, C18 silica gel; mobile phase, MeCN in water, 20% to 50% gradient in 30 min; detector, UV 254 nm. to afford 3-([[4-iodo-6-(morpholin-4-yl)pyridin-2-yl]amino]methyl)oxetan-3-ol (148 mg, 23%) as a light brown solid. MS ESI calculated for C₁₃H₁₈IN₃O₃[M+H]⁺, 392.04, found 392.00. ¹H NMR (400 MHz, DMSO-d₆) δ 6.56-6.53 (m, 1H), 6.33 (m, 1H), 6.23 (m, 1H), 5.90 (s, 1H), 4.41-4.36 (m, 4H), 3.66-3.63 (m, 4H), 3.54-3.53 (m, 2H), 3.36-3.34 (m, 4H).

### Example 6: (3S)-N-[3-(2-[[(3-hydroxyoxetan-3-yl)methyl]amino]-6-(morpholin-4-yl)pyridin-4-yl)-4-methylphenyl]-3-(2,2,2-trifluoroethyl)pyrrolidine-1-carboxamide

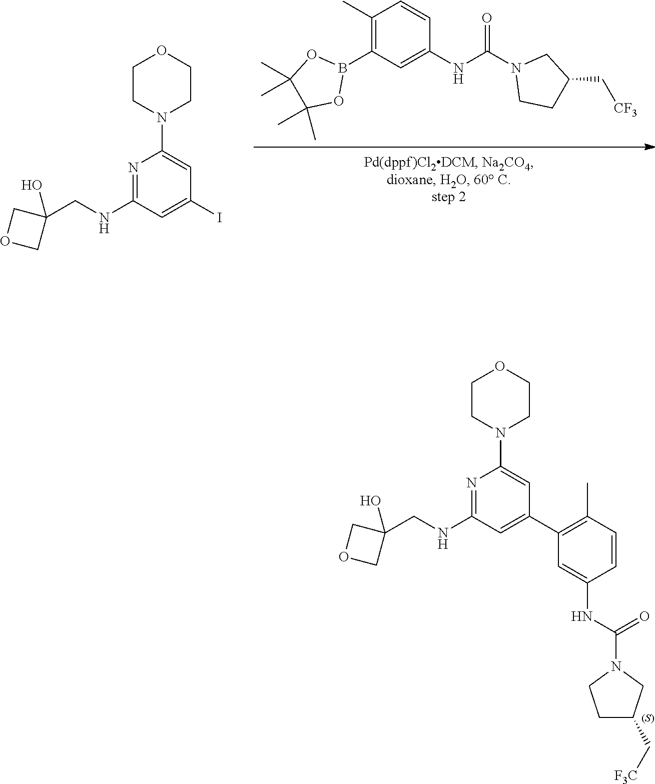

To a stirred solution of 3-([[4-iodo-6-(morpholin-4-yl)pyridin-2-yl]amino]methyl)oxetan-3-ol (140 mg, 0.358 mmol), (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (162 mg, 0.394 mmol) and Na₂CO₃ (114 mg, 1.074 mmol) in dioxane:H₂O=4:1 (2.00 mL) was added Pd(dppf)Cl₂·CH₂Cl₂ (29 mg, 0.036 mmol) at 25° C. under nitrogen atmosphere. The resulting mixture was stirred for 16 h at 60° C. under nitrogen atmosphere. The reaction was quenched by the addition of water (100 mL). The resulting mixture was extracted with EtOAc (3×50 mL). The combined organic layers were washed with saturated brine (3×50 mL), dried over anhydrous Na₂SO₄. After filtration, the filtrate was concentrated under reduced pressure. The residue was purified by silica gel column chromatography, eluted with PE/(EA/EtOH=3:1) (2:1) to afford 150 mg crude product. The residue was purified by reverse flash chromatography with the following conditions: Column: XBridge Shield RP18 OBD Column, 5 um, 19*150 mm; Mobile Phase A:Water(10 MMOL/L NH₄HCO₃), Mobile Phase B: CH₃CN; Flow rate:20 mL/min; Gradient:45 B to 60 B in 4.5 min; 254/210 nm to afford (3S)-N-[3-(2-[[(3-hydroxyoxetan-3-yl)methyl]amino]-6-(morpholin-4-yl)pyridin-4-yl)-4-methylphenyl]-3-(2,2,2-trifluoroethyl)pyrrolidine-1-carboxamide (100 mg, 51%) as an off-white solid. MS ESI calculated for C₂₇H₃₄F₃N₅O₄[M+H]⁺, 550.26, found 550.25. ¹H NMR (300 MHz, DMSO-d₆) δ 8.11 (s, 1H), 7.44-7.35 (m, 2H), 7.11-7.09 (m, 1H), 6.45-6.42 (m, 1H), 6.05 (s, 1H), 5.86-5.81 (m, 2H), 4.46-4.39 (m, 4H), 3.69-3.64 (m, 5H), 3.61-3.59 (m, 2H), 3.55-3.50 (m, 1H), 3.39-3.34 (m, 4H), 3.30-3.27 (m, 1H), 3.05-3.00 (m, 1H), 2.48-2.36 (m, 3H), 2.16 (s, 3H), 2.10-2.07 (m, 1H), 1.71-1.61 (m, 1H). ¹⁹F NMR (376 MHz, DMSO-d₆) δ-63.38 (3F).

### Example 7: (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide

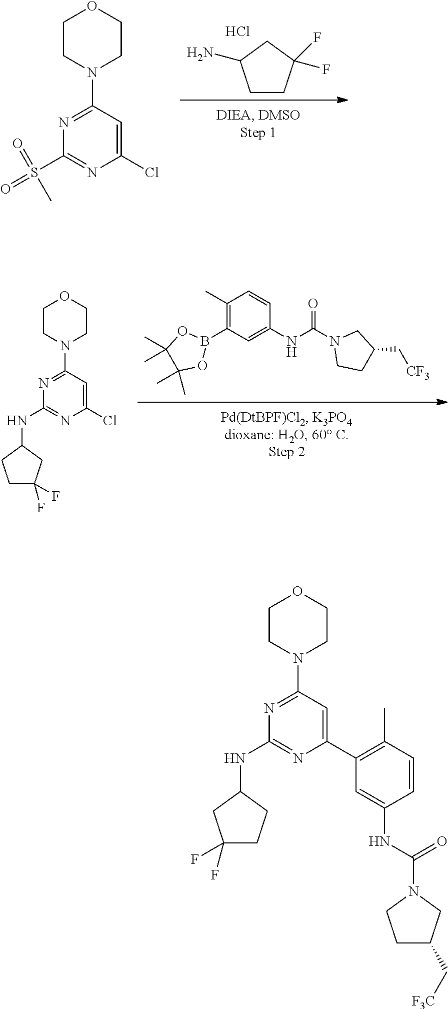 [SMILES: CS(=O)(=O)c1nc(Cl)cc(N2CCOCC2)n1 ; NC1CCC(F)(F)C1 ; Cl ; FC1(F)CCC(Nc2nc(Cl)cc(N3CCOCC3)n2)C1 ; Cc1ccc(NC(=O)N2CC[C@@H](CC(F)(F)F)C2)cc1B1OC(C)(C)C(C)(C)O1 ; Cc1ccc(NC(=O)N2CC[C@@H](CC(F)(F)F)C2)cc1-c1cc(N2CCOCC2)nc(NC2CCC(F)(F)C2)n1]

Preparation 7A: 4-chloro-N-(3,3-difluorocyclopentyl)-6-(morpholin-4-yl)pyrimidin-2-amine

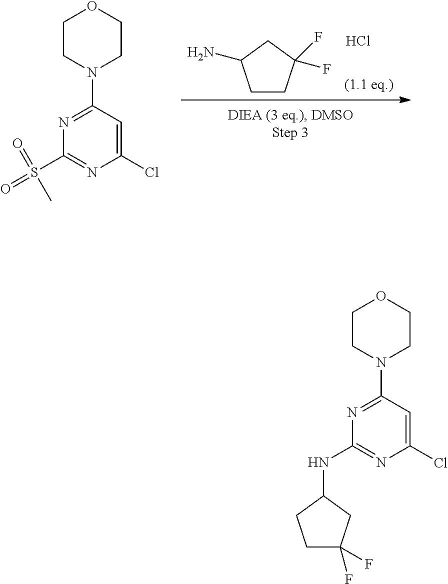 [SMILES: CS(=O)(=O)c1nc(Cl)cc(N2CCOCC2)n1 ; NC1CCC(F)(F)C1 ; Cl ; FC1(F)CCC(Nc2nc(Cl)cc(N3CCOCC3)n2)C1]

To a stirred solution of 4-(6-chloro-2-methanesulfonylpyrimidin-4-yl)morpholine (200 mg, 0.720 mmol) and 3,3-difluorocyclopentan-1-amine hydrochloride (125 mg, 0.79 mmol) in DMSO (4 mL) was added DIEA (279 mg, 2.16 mmol) dropwise at room temperature under nitrogen atmosphere. The resulting mixture was stirred for overnight at 60° C. under nitrogen atmosphere. The resulting mixture was diluted with water (15 mL). The resulting mixture was extracted with EtOAc (3×20 mL). The combined organic layers were washed with brine (15 mL), dried over anhydrous Na₂SO₄. After filtration, the filtrate was concentrated under reduced pressure. The residue was purified by silica gel column chromatography, eluted with hexane/EtOAc (1:1) to afford 4-chloro-N-(3,3-difluorocyclopentyl)-6-(morpholin-4-yl)pyrimidin-2-amine (84 mg, 37%) as a white solid. MS ESI calculated for C₁₃H₁₇ClF₂N₄O [M+H]⁺, 319.11; found 319.15. ¹H NMR (300 MHz, DMSO-d₆) δ 5.93 (s, 1H), 5.16 (s, 1H), 4.47-4.42 (m, 1H), 3.79-3.76 (m, 4H), 3.60-3.57 (m, 4H), 2.70-2.53 (m, 1H), 2.32-1.82 (m, 4H), 1.79-1.73 (m, 1H).

### Example 7: (3S)-N-(3-[2-[(3,3-difluorocyclopentyl)amino]-6-(morpholin-4-yl)pyrimidin-4-yl]-4-methylphenyl)-3-(2,2,2-trifluoroethyl)pyrrolidine-1-carboxamide

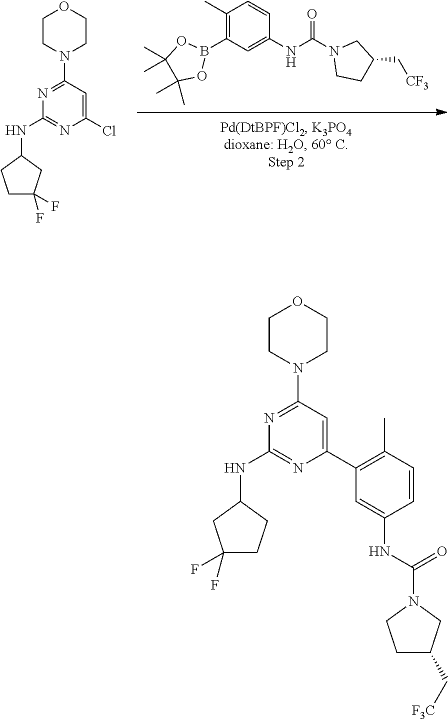 [SMILES: FC1(F)CCC(Nc2nc(Cl)cc(N3CCOCC3)n2)C1 ; Cc1ccc(NC(=O)N2CC[C@@H](CC(F)(F)F)C2)cc1B1OC(C)(C)C(C)(C)O1 ; Cc1ccc(NC(=O)N2CC[C@@H](CC(F)(F)F)C2)cc1-c1cc(N2CCOCC2)nc(NC2CCC(F)(F)C2)n1]

To a stirred solution of 4-chloro-N-(3,3-difluorocyclopentyl)-6-(morpholin-4-yl)pyrimidin-2-amine (160 mg, 0.502 mmol) and (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (248 mg, 0.602 mmol), K₃PO₄ (213 mg, 1.004 mmol) in dioxane (3.2 mL), H₂O (0.8 mL) was added Pd(DtBPF)Cl₂ (33 mg, 0.050 mmol) at room temperature under nitrogen atmosphere. The resulting mixture was stirred for 2 h at 80° C. under nitrogen atmosphere. The resulting mixture was concentrated under vacuum. The residue was purified by silica gel column chromatography, eluted with hexane/(EtOAc:EtOH=3:1) (1:1) to afford crude. The crude product (150 mg) was purified by Prep-HPLC with the following conditions (Column: XSelect CSH Prep C18 OBD Column, 19×250 mm, 5 um; Mobile Phase A: Water (0.1% FA), Mobile Phase B: CH₃CN; Flow rate: 20 mL/min; Gradient: 30 B to 40 B in 6.5 min; 254/210 nm to afford (3S)-N-(3-[2-[(3,3-difluorocyclopentyl)amino]-6-(morpholin-4-yl)pyrimidin-4-yl]-4-methylphenyl)-3-(2,2,2-trifluoroethyl)pyrrolidine-1-carboxamide (85 mg, 30%) as a white solid. MS ESI calculated for C₂₇H₃₃F₅N₆O₂[M+H]⁺, 569.26, found 569.20. ¹H NMR (400 MHz, DMSO-d₆) δ 8.14 (s, 1H), 7.47-7.45 (m, 2H), 7.11-7.09 (m, 2H), 6.07 (s, 1H), 4.37-4.35 (m, 1H), 3.67-3.51 (m, 10H), 3.05-3.00 (m, 1H), 2.44-2.42 (m, 6H), 2.34-2.21 (m, 3H), 2.09-2.08 (m, 4H), 1.79-1.63 (m, 2H). ¹⁹F NMR (376 MHz, DMSO-d6) δ-63.37 (3F), −87.50 (2F).

### Examples 8 and 9: (3S)-N-[3-(2-[[(1R)-3,3-difluorocyclopentyl]amino]-6-(morpholin-4-yl)pyrimidin-4-yl)-4-methylphenyl]-3-(2,2,2-trifluoroethyl)pyrrolidine-1-carboxamide, (3S)-N-[3-(2-[[(1S)-3,3-difluorocyclopentyl]amino]-6-(morpholin-4-yl)pyrimidin-4-yl)-4-methylphenyl]-3-(2,2,2-trifluoroethyl)pyrrolidine-1-carboxamide

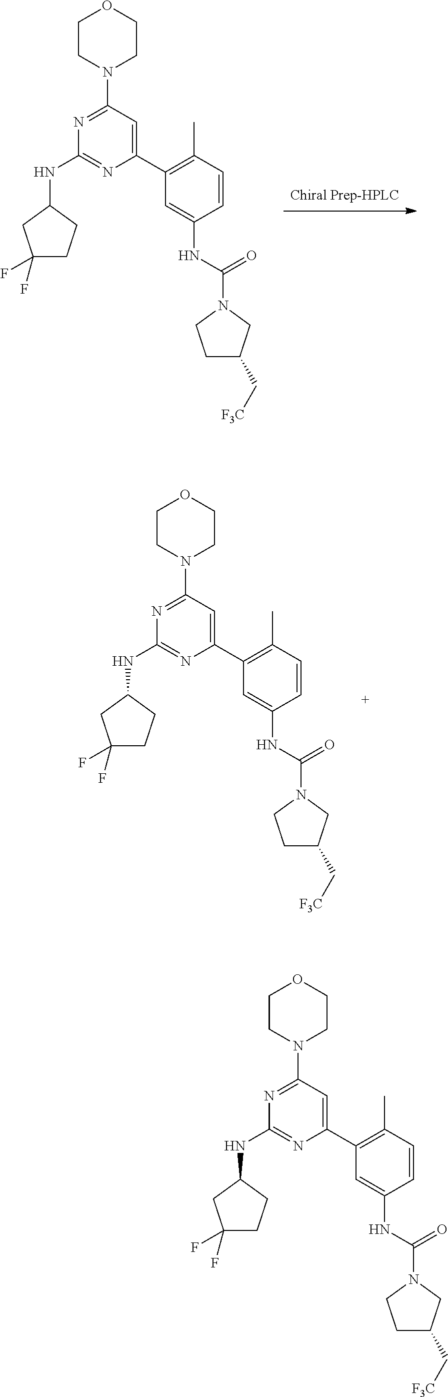 [SMILES: Cc1ccc(NC(=O)N2CC[C@@H](CC(F)(F)F)C2)cc1-c1cc(N2CCOCC2)nc(NC2CCC(F)(F)C2)n1 ; Cc1ccc(NC(=O)N2CC[C@@H](CC(F)(F)F)C2)cc1-c1cc(N2CCOCC2)nc(N[C@@H]2CCC(F)(F)C2)n1 ; Cc1ccc(NC(=O)N2CC[C@@H](CC(F)(F)F)C2)cc1-c1cc(N2CCOCC2)nc(N[C@H]2CCC(F)(F)C2)n1]

(3S)-N-(3-[2-[(3,3-difluorocyclopentyl)amino]-6-(morpholin-4-yl)pyrimidin-4-yl]-4-methylphenyl)-3-(2,2,2-trifluoroethyl)pyrrolidine-1-carboxamide (80 mg) was resolved by Prep-CHIRAL-HPLC with the following conditions (Column: CHIRALPAK ADH, 2×25 cm, 5 um; Mobile Phase A: Hex (8 mmol/L NH₃·MeOH)-HPLC, Mobile Phase B: IPA-HPLC; Flow rate: 20 mL/min; Gradient: 20 B to 20 B in 35 min; 220/254 nm) to afford 33 mg (41%) as a white solid. MS ESI calculated for C₂₇H₃₃F₅N₆O₂ [M+H]⁺, 569.26, found 569.25. ¹H NMR (400 MHz, DMSO-d₆) δ 8.13 (s, 1H), 7.47-7.44 (m, 2H), 7.10-7.08 (m, 2H), 6.06 (s, 1H), 4.36-4.34 (m, 1H), 3.69-3.51 (m, 11H), 3.05-3.00 (m, 1H), 2.47-2.27 (m, 7H), 2.11-2.08 (m, 5H), 1.75-1.66 (m, 2H). ¹⁹F NMR (376 MHz, DMSO-d₆) δ-63.37 (3F), −87.50 (2F), and 33 mg (41%) as a white solid. MS ESI calculated for C₂₇H₃₃F₅N₆O₂ [M+H]⁺, 569.26, found 569.25. ¹H NMR (400 MHz, DMSO-d₆) δ 8.13 (s, 1H), 7.47-7.44 (m, 2H), 7.10-7.08 (m, 1H), 6.93-6.82 (m, 1H), 6.06 (s, 1H), 4.39-4.27 (m, 1H), 3.66-3.55 (m, 11H), 3.05-3.00 (m, 1H), 2.47-2.27 (m, 7H), 2.19-2.03 (m, 5H), 1.79-1.66 (m, 2H). ¹⁹F NMR (376 MHz, DMSO-d₆) δ-63.37 (3F), −87.51 (2F).

### Example 10: (3S)-N-[3-(2-[[(cis-3-hydroxycyclopentyl]amino]-6-(morpholin-4-yl)pyrimidin-4-yl)-4-methylphenyl]-3-(2,2,2-trifluoroethyl)pyrrolidine-1-carboxamide

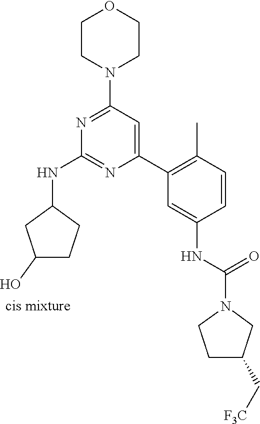 [SMILES: Cc1ccc(NC(=O)N2CC[C@@H](CC(F)(F)F)C2)cc1-c1cc(N2CCOCC2)nc(NC2CCC(O)C2)n1]

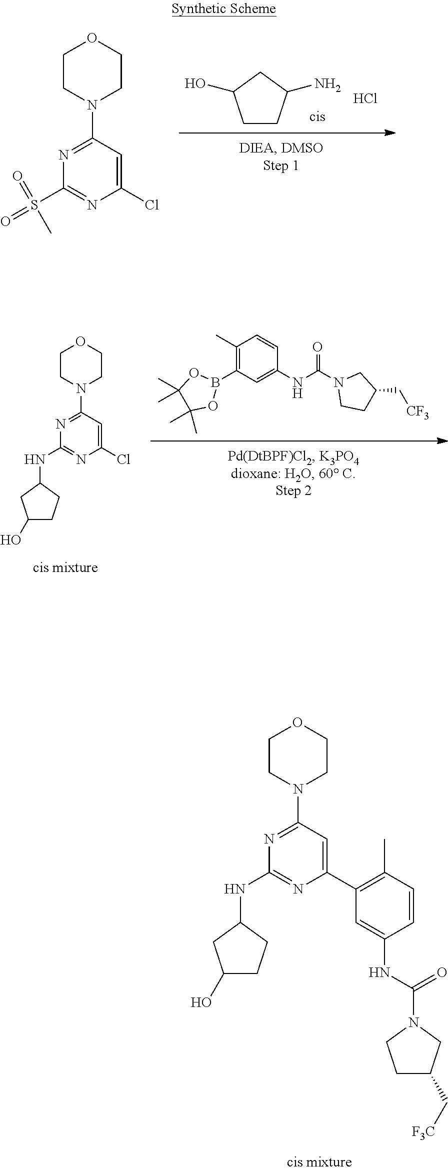 [SMILES: CS(=O)(=O)c1nc(Cl)cc(N2CCOCC2)n1 ; NC1CCC(O)C1 ; Cl ; OC1CCC(Nc2nc(Cl)cc(N3CCOCC3)n2)C1 ; Cc1ccc(NC(=O)N2CC[C@@H](CC(F)(F)F)C2)cc1B1OC(C)(C)C(C)(C)O1 ; Cc1ccc(NC(=O)N2CC[C@@H](CC(F)(F)F)C2)cc1-c1cc(N2CCOCC2)nc(NC2CCC(O)C2)n1]

Preparation 10A: cis-3-[[4-chloro-6-(morpholin-4-yl)pyrimidin-2-yl]amino]cyclopentan-1-ol

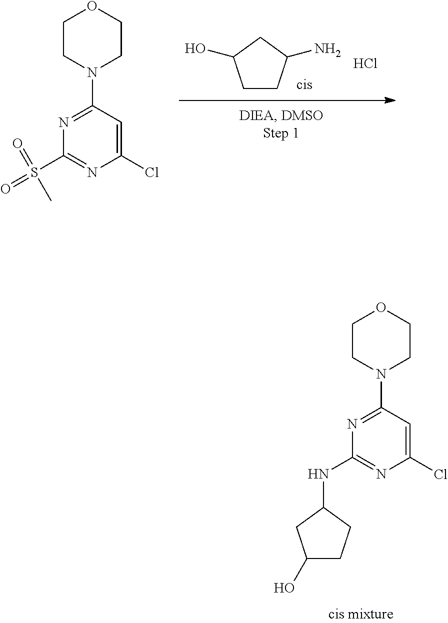 [SMILES: CS(=O)(=O)c1nc(Cl)cc(N2CCOCC2)n1 ; NC1CCC(O)C1 ; Cl ; OC1CCC(Nc2nc(Cl)cc(N3CCOCC3)n2)C1]

To a stirred solution of 4-(6-chloro-2-methanesulfonylpyrimidin-4-yl)morpholine (500 mg, 1.800 mmol) and cis-3-aminocyclopentan-1-ol hydrochloride (273 mg, 1.980 mmol) in DMSO (9 mL) were added DIEA (698 mg, 5.401 mmol) dropwise at room temperature under nitrogen atmosphere. The resulting mixture was stirred for overnight at 60° C. under nitrogen atmosphere. The resulting mixture was diluted with water (30 mL). The resulting mixture was extracted with EtOEt (4×30 mL). The combined organic layers were washed with brine (30 mL), dried over anhydrous Na₂SO₄. After filtration, the filtrate was concentrated under reduced pressure. The residue was purified by silica gel column chromatography, eluted with hexane/(EtOAc:EtOH=3:1) (1:1) to afford (cis)-3-[[4-chloro-6-(morpholin-4-yl)pyrimidin-2-yl]amino]cyclopentan-1-ol (221 mg, 42%) as a white solid. MS ESI calculated for C₁₃H₁₉ClN₄O₂ [M+H]⁺, 299.12; found 299.15 ¹H NMR (300 MHz, chloroform-d) δ 5.88-5.87 (m, 1H), 5.57 (m, 1H), 5.34-5.32 (m, 1H). 4.43-4.35 (m, 2H), 3.78-3.75 (m, 4H), 3.60-3.56 (m, 4H), 2.23-2.07 (m, 2H), 1.94-1.80 (m, 4H).

### Example 10: (3S)-N-[3-(2-[[(cis-3-hydroxycyclopentyl]amino]-6-(morpholin-4-yl)pyrimidin-4-yl)-4-methylphenyl]-3-(2,2,2-trifluoroethyl)pyrrolidine-1-carboxamide

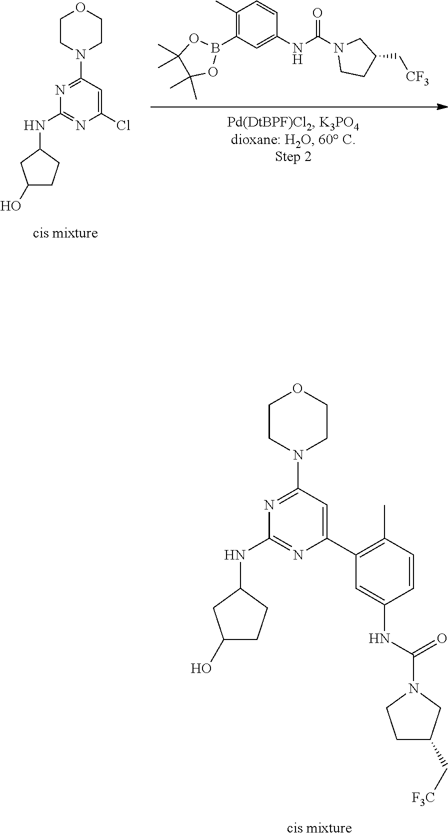 [SMILES: OC1CCC(Nc2nc(Cl)cc(N3CCOCC3)n2)C1 ; Cc1ccc(NC(=O)N2CC[C@@H](CC(F)(F)F)C2)cc1B1OC(C)(C)C(C)(C)O1 ; Cc1ccc(NC(=O)N2CC[C@@H](CC(F)(F)F)C2)cc1-c1cc(N2CCOCC2)nc(NC2CCC(O)C2)n1]

To a stirred mixture of (cis)-3-[[4-chloro-6-(morpholin-4-yl)pyrimidin-2-yl]amino]cyclopentan-1-ol (270 mg, 0.904 mmol) and (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (447 mg, 1.084 mmol), K₃PO₄ (384 mg, 1.807 mmol) in dioxane (5.4 mL), H₂O (1.35 mL) were added Pd(DtBPF)Cl₂ (59 mg, 0.090 mmol) at room temperature under nitrogen atmosphere. The resulting mixture was stirred for 2 h at 80° C. under nitrogen atmosphere. The resulting mixture was concentrated under vacuum. The residue was purified by silica gel column chromatography, eluted with hexane/(EtOAc:EtOH=3:1) (1:1) to afford crude. The crude product (277 mg) was purified by Prep-HPLC with the following conditions (Column: XBridge Shield RP18 OBD Column, 5 μm, 19×150 mm; Mobile Phase A: Water (10 MMOL/L NH₄HCO₃), Mobile Phase B: CH₃CN; Flow rate:20 mL/min; Gradient: 28 B to 40 B in 4.3 min; 254/210 nm to afford (3S)-N-[3-(2-[[(cis)-3-hydroxycyclopentyl]amino]-6-(morpholin-4-yl)pyrimidin-4-yl)-4-methylphenyl]-3-(2,2,2-trifluoroethyl)pyrrolidine-1-carboxamide (165 mg, 33%) as a white solid. MS ESI calculated for C₂₇H₃₅F₃N₆O₃ [M+H]⁺, 549.27; found 549.30. ¹H NMR (300 MHz, DMSO-d₆) δ 8.12 (s, 1H), 7.44-7.42 (m, 2H), 7.08-7.06 (m, 1H), 6.41-6.39 (m, 1H), 6.00 (s, 1H), 4.60-5.59 (m, 1H), 4.17-4.08 (m, 2H), 3.69-3.42 (m, 10H), 3.05-2.99 (m, 1H), 2.46-2.39 (m, 3H), 2.24-2.15 (m, 3H), 2.10-2.07 (m, 3H), 1.88-1.85 (m, 1H), 1.82-1.77 (m, 5H). ¹⁹F NMR (282 MHz, DMSO-d₆) δ-63.35 (3F).

### Examples 11 and 12: (3S)-N-[3-(2-1[[(1S,3R)-3-hydroxycyclopentyl]amino]-6-(morpholin-4-yl)pyrimidin-4-yl)-4-methylphenyl]-3-(2,2,2-trifluoroethyl)pyrrolidine-1-carboxamide, (3S)-N-[3-(2-[[(1R,3S)-3-hydroxycyclopentyl]amino]-6-(morpholin-4-yl)pyrimidin-4-yl)-4-methylphenyl]-3-(2,2,2-trifluoroethyl)pyrrolidine-1-carboxamide

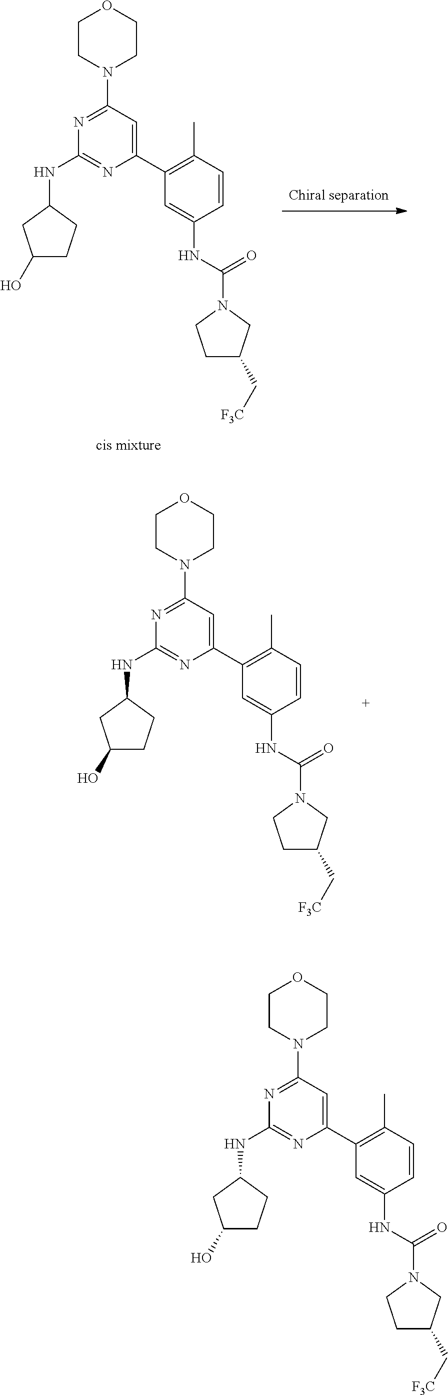 [SMILES: Cc1ccc(NC(=O)N2CC[C@@H](CC(F)(F)F)C2)cc1-c1cc(N2CCOCC2)nc(NC2CCC(O)C2)n1 ; Cc1ccc(NC(=O)N2CC[C@@H](CC(F)(F)F)C2)cc1-c1cc(N2CCOCC2)nc(N[C@H]2CC[C@@H](O)C2)n1 ; Cc1ccc(NC(=O)N2CC[C@@H](CC(F)(F)F)C2)cc1-c1cc(N2CCOCC2)nc(N[C@@H]2CC[C@H](O)C2)n1]

(3S)-N-[3-(2-[[(cis)-3-hydroxycyclopentyl]amino]-6-(morpholin-4-yl)pyrimidin-4-yl)-4-methylphenyl]-3-(2,2,2-trifluoroethyl)pyrrolidine-1-carboxamide (160 mg) was resolved by Prep-CHIRAL-HPLC with the following conditions (Column: CHIRALPAK ADH, 2×25 cm, 5 um; Mobile Phase A: Hex (8 mmol/L NH₃·MeOH)-HPLC, Mobile Phase B:IPA-HPLC; Flow rate: 20 mL/min; Gradient: 30 B to 30 B in 15 min; 220/254 nm) to afford 66 mg (41%) as a white solid. MS ESI calculated for C₂₇H₃₅F₃N₆O₃ [M+H]⁺, 549.27; found 549.30. ¹H NMR (400 MHz, DMSO-d₆) δ 8.15-8.13 (m, 1H), 7.49-7.46 (m, 2H), 7.11-7.08 (m, 1H), 6.52-6.33 (m, 1H), 6.01 (s, 1H), 4.63-4.61 (m, 1H), 4.23-4.02 (m, 2H), 3.79-3.52 (m, 11H), 3.13-3.02 (m, 1H), 2.46-2.44 (m, 3H), 2.39-2.12 (m, 3H), 2.17-2.05 (m, 2H), 1.93-1.86 (m, 1H), 1.73-1.46 (m, 5H). ¹⁹F NMR (376 MHz, DMSO-d₆) δ-63.37 (3F), and 67 mg (42%) as a white solid. MS ESI calculated for C₂₇H₃₅F₃N₆O₃[M+H]⁺, 549.27; found 549.30. ¹H NMR (400 MHz, DMSO-d₆) δ 8.15-8.13 (m, 1H), 7.57-7.40 (m, 2H), 7.11-7.08 (m, 1H), 6.52-6.43 (m, 1H), 6.10-5.96 (m, 1H), 4.63-5.61 (m, 1H), 4.17-4.10 (m, 2H), 3.82-3.41 (m, 11H), 3.10-3.02 (m, 1H), 2.46-2.39 (m, 3H), 2.24-2.19 (m, 3H), 2.17-2.07 (m, 2H), 1.88-1.68 (m, 1H), 1.65-1.46 (m, 5H). ¹⁹F NMR (376 MHz, DMSO-d₆) δ -63.34-63.37 (3F).

### Example 13: (3S)-N-[3-(2-[[(2R)-2-hydroxypropyl]amino]-6-(morpholin-4-yl)pyridin-4-yl)-4-methylphenyl]-3-(2,2,2-trifluoroethyl)pyrrolidine-1-carboxamide

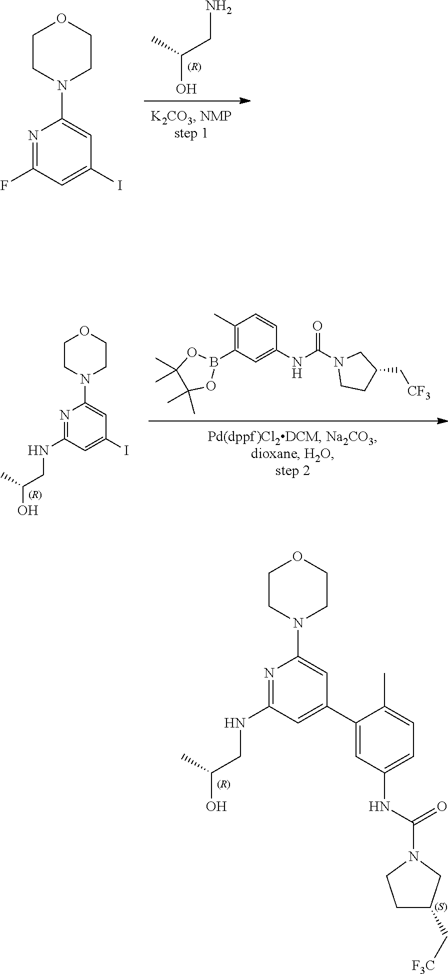

Preparation 13A: (2R)-1-[[4-iodo-6-(morpholin-4-yl)pyridin-2-yl]amino]propan-2-ol

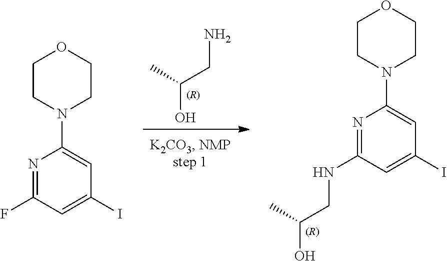

To a stirred solution of 4-(6-fluoro-4-iodopyridin-2-yl)morpholine (400 mg, 1.298 mmol) and K₂CO₃ (359 mg, 2.597 mmol) in NMP (4 mL) was added (R)-1-amino-2-propanol (146 mg, 1.947 mmol) at room temperature under nitrogen atmosphere. The resulting mixture was stirred for 16 h at 150° C. under nitrogen atmosphere. The reaction was quenched by the addition of water (100 mL). The resulting mixture was extracted with EtOAc (3×50 mL). The combined organic layers were washed with saturated brine (3×50 mL), dried over anhydrous Na₂SO₄. After filtration, the filtrate was concentrated under reduced pressure. The residue was purified by silica gel column chromatography, eluted with PE/EtOAc (2:1) to afford (2R)-1-[[4-iodo-6-(morpholin-4-yl)pyridin-2-yl]amino]propan-2-ol (200 mg, 42%) as an off-white solid. MS ESI calculated for C₁₂H₁₈IN₃O₂[M+H]⁺, 364.04, found 363.90. ¹H NMR (400 MHz, DMSO-d₆) δ 6.37 (m, 1H), 6.25 (m, 1H), 6.19 (m, 1H), 4.64-4.63 (m, 1H), 3.77-3.71 (m, 1H), 3.66-3.63 (m, 4H), 3.34-3.33 (m, 4H), 3.17-3.06 (m, 2H), 1.06-1.04 (m, 3H).

### Example 13: (3S)-N-[3-(2-[[(2R)-2-hydroxypropyl]amino]-6-(morpholin-4-yl)pyridin-4-yl)-4-methylphenyl]-3-(2,2,2-trifluoroethyl)pyrrolidine-1-carboxamide

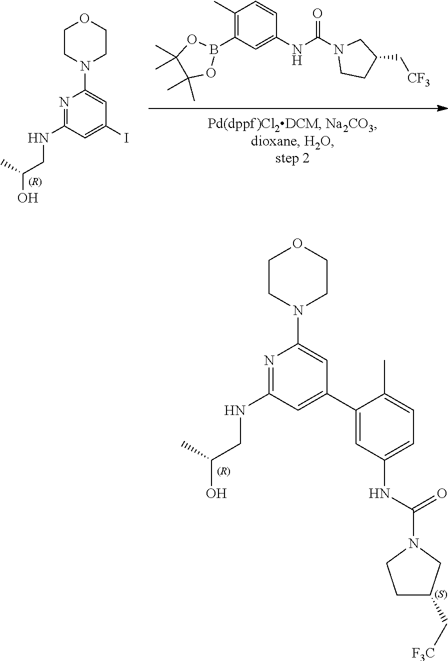

To a stirred solution of (2R)-1-[[4-iodo-6-(morpholin-4-yl)pyridin-2-yl]amino]propan-2-ol (50 mg, 0.138 mmol), (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (60 mg, 0.145 mmol) and Na₂CO₃ (44 mg, 0.413 mmol) in dioxane (0.8 mL) and H₂O (0.2 mL) was added Pd(dppf)Cl₂·CH₂Cl₂ (11 mg, 0.014 mmol) at room temperature under nitrogen atmosphere. The resulting mixture was stirred for 16 h at 60° C. under nitrogen atmosphere. The reaction was quenched by the addition of water (100 mL). The resulting mixture was extracted with EtOAc (3×50 mL). The combined organic layers were washed with saturated brine (3×50 mL), dried over anhydrous Na₂SO₄. After filtration, the filtrate was concentrated under reduced pressure. The residue was purified by silica gel column chromatography, eluted with PE/EA/EtOH (8/3/1) to afford 50 mg crude product. The residue was purified by reverse flash chromatography with the following conditions: Column: XBridge Prep Amide OBD Column, 19×150 mm Sum 13 nm; Mobile Phase A:Water(10 mmol/L NH₄HCO₃), Mobile Phase B: CH₃CN; Flow rate:20 mL/min; Gradient:40 B to 70 B in 4.3 min; 210/254 nm. This resulted in (3S)-N-[3-(2-[[(2R)-2-hydroxypropyl]amino]-6-(morpholin-4-yl)pyridin-4-yl)-4-methylphenyl]-3-(2,2,2-trifluoroethyl)pyrrolidine-1-carboxamide (38 mg, 53%) as an off-white solid. MS ESI calculated for C₂₆H₃₄F₃N₅O₃[M+H]⁺. 522.26, found 522.15. ¹H NMR (400 MHz, DMSO-d₆) δ 8.11 (s, 1H), 7.43-7.40 (m, 1H), 7.34-7.33 (m, 1H), 7.10-7.08 (m, 1H), 6.22-6.19 (m, 1H), 5.78-5.76 (m, 2H), 4.70-4.69 (m, 1H), 3.83-3.77 (m, 1H), 3.69-3.55 (m, 5H), 3.53-3.50 (m, 1H), 3.38-3.36 (m, 4H), 3.31-3.27 (m, 1H), 3.22-3.13 (m, 2H), 3.04-3.00 (m, 1H), 2.48-2.38 (m, 3H), 2.16 (s, 3H), 2.11-2.07 (m, 1H), 1.68-1.63 (m, 1H), 1.09-1.07 (m, 3H). ¹⁹F NMR (376 MHz, DMSO-d₆) δ-63.36 (3F).

### Example 14: (3S)-N-[3-(2-[[(2R)-1-hydroxypropan-2-yl]amino]-6-(morpholin-4-yl)pyridin-4-yl)-4-methylphenyl]-3-(2,2,2-trifluoroethyl)pyrrolidine-1-carboxamide

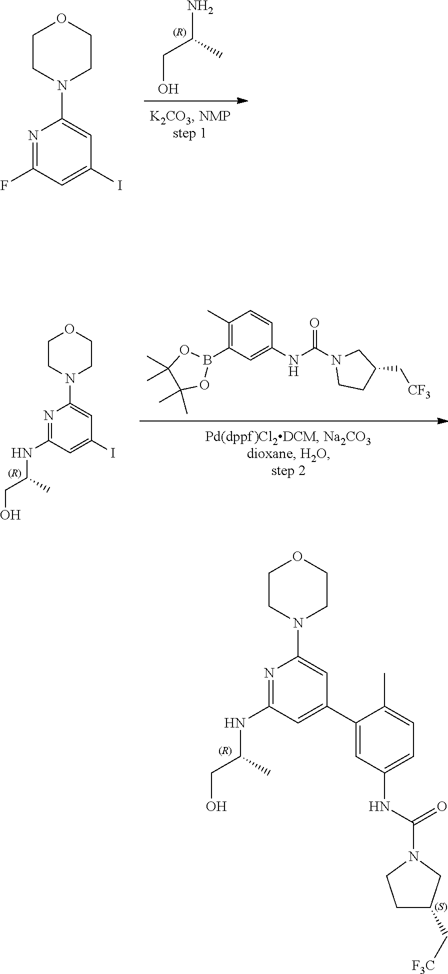

Preparation 14A: (2R)-2-[[4-iodo-6-(morpholin-4-yl)pyridin-2-yl]amino]propan-1-ol

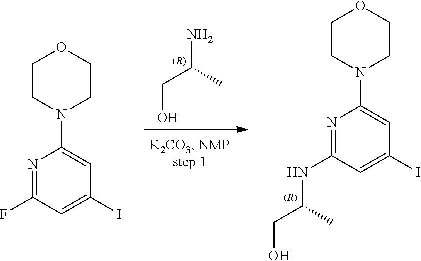

To a stirred solution of 4-(6-fluoro-4-iodopyridin-2-yl)morpholine (300 mg, 0.974 mmol) and K₂CO₃ (269 mg, 1.947 mmol) in NMP (3 mL) was added (R)-(−)-2-amino-1-propanol (110 mg, 1.461 mmol) at room temperature under nitrogen atmosphere. The resulting mixture was stirred for 16 h at 150° C. under nitrogen atmosphere. The reaction was quenched by the addition of water (100 mL). The resulting mixture was extracted with EtOAc (3×50 mL). The combined organic layers were washed with saturated brine (3×50 mL), dried over anhydrous Na₂SO₄. After filtration, the filtrate was concentrated under reduced pressure. The residue was purified by silica gel column chromatography, eluted with PE/EtOAc (2:1) to afford (2R)-2-[[4-iodo-6-(morpholin-4-yl)pyridin-2-yl]amino]propan-1-ol (85 mg, 24%) as an off-white solid. MS ESI calculated for C₁₂H₁₈IN₃O₂[M+H]⁺, 364.04, found 364.10. ¹H NMR (400 MHz, DMSO-d₆) δ 6.23 (s, 1H), 6.18-6.13 (m, 2H), 4.63-4.60 (m, 1H), 3.86-3.80 (m, 1H), 3.66-3.63 (m, 4H), 3.45-3.41 (m, 1H), 3.38-3.34 (m, 4H), 3.28-3.22 (m, 1H), 1.09-1.07 (m, 3H).

### Example 14: (3S)-N-[3-(2-[[(2R)-1-hydroxypropan-2-yl]amino]-6-(morpholin-4-yl)pyridin-4-yl)-4-methylphenyl]-3-(2,2,2-trifluoroethyl)pyrrolidine-1-carboxamide

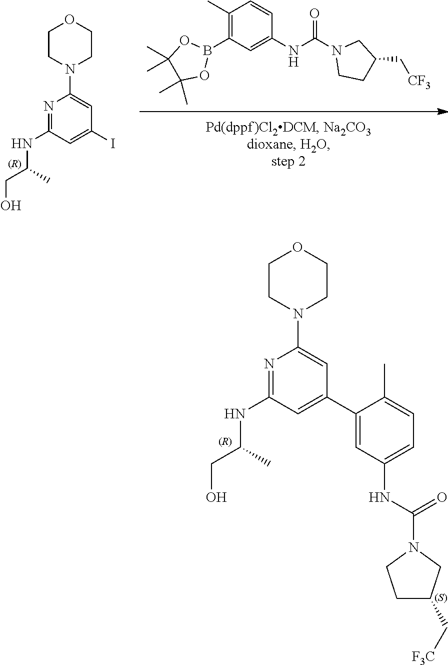

To a stirred solution of (2R)-2-[[4-iodo-6-(morpholin-4-yl)pyridin-2-yl]amino]propan-1-ol (50 mg, 0.138 mmol), (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (57 mg, 0.138 mmol) and Na₂CO₃ (44 mg, 0.413 mmol) in dioxane (0.8 mL) and H₂O (0.2 mL) was added Pd(dppf)Cl₂·CH₂Cl₂ (11 mg, 0.014 mmol) at room temperature under nitrogen atmosphere. The resulting mixture was stirred for 16 h at 60° C. under nitrogen atmosphere. The reaction was quenched by the addition of water (100 mL). The resulting mixture was extracted with EtOAc (3×50 mL). The combined organic layers were washed with saturated brine (3×50 mL), dried over anhydrous Na₂SO₄. After filtration, the filtrate was concentrated under reduced pressure. The residue was purified by silica gel column chromatography, eluted with PE/EA/EtOH (8/3/1) to afford 50 mg crude product. The residue was purified by reverse flash chromatography with the following conditions: Column: XBridge Shield RP18 OBD Column, 30*150 mm, 5 um; Mobile Phase A:Water(10 mmol/l NH₄HCO₃), Mobile Phase B: CH₃CN; Flow rate:20 mL/min; Gradient:35 B to 65 B in 8 min; 210/254 nm. This resulted in (3S)-N-[3-(2-[[(2R)-1-hydroxypropan-2-yl]amino]-6-(morpholin-4-yl)pyridin-4-yl)-4-methylphenyl]-3-(2,2,2-trifluoroethyl)pyrrolidine-1-carboxamide (31 mg, 43%) as an off-white solid. MS ESI calculated for C₂₆H₃₄F₃NsO₃ [M+H]⁺. 522.26, found 522.10. ¹H NMR (400 MHz, DMSO-d₆) δ 8.11 (s, 1H), 7.42-7.40 (m, 1H), 7.34 (m, 1H), 7.10-7.08 (m, 1H), 5.98-5.96 (m, 1H), 5.75 (m, 2H), 4.66-4.64 (m, 1H), 3.91-3.87 (m, 1H), 3.69-3.65 (m, 5H), 3.52-3.47 (m, 2H), 3.38-3.36 (m, 4H), 3.32-3.26 (m, 2H), 3.04-3.00 (m, 1H), 2.48-2.38 (m, 3H), 2.16 (s, 3H), 2.10-2.07 (m, 1H), 1.71-1.61 (m, 1H), 1.13-1.12 (m, 3H). ¹⁹F NMR (376 MHz, DMSO-d₆) δ-63.36 (3F).

### Example 15: (3S)-N-[3-[2-(3-hydroxyazetidin-1-yl)-6-(morpholin-4-yl)pyrimidin-4-yl]-4-methylphenyl]-3-(2,2,2-trifluoroethyl)pyrrolidine-1-carboxamide

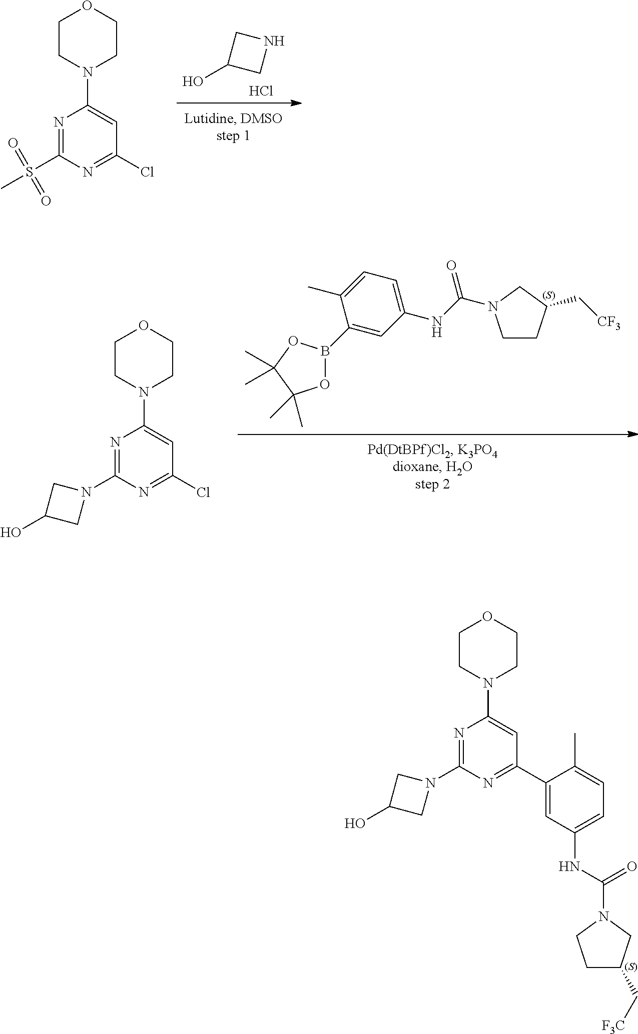

Preparation 15A: 1-[4-chloro-6-(morpholin-4-yl)pyrimidin-2-yl]azetidin-3-ol

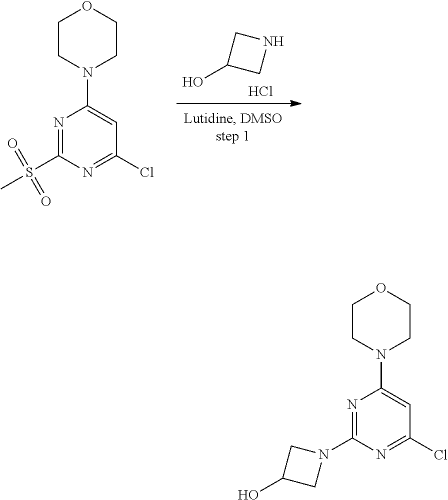 [SMILES: CS(=O)(=O)c1nc(Cl)cc(N2CCOCC2)n1 ; OC1CNC1 ; Cl ; OC1CN(c2nc(Cl)cc(N3CCOCC3)n2)C1]

A mixture of 4-(6-chloro-2-methanesulfonylpyrimidin-4-yl)morpholine (200 mg, 0.720 mmol), azetidin-3-ol hydrochloride (87 mg, 0.792 mmol) and 2,6-Dimethylpyridine (232 mg, 2.160 mmol) in DMSO (2 mL) was stirred for 16 h at 60° C. under nitrogen atmosphere. The reaction was quenched by the addition of Water (20 mL) at room temperature. The resulting mixture was extracted with EtOAc (5×20 mL). The combined organic layers were dried over anhydrous Na₂SO₄. After filtration, the filtrate was concentrated under reduced pressure. The residue was purified by silica gel column chromatography, eluted with PE/EtOAc/EtOH (4/3/1) to afford 1-[4-chloro-6-(morpholin-4-yl)pyrimidin-2-yl]azetidin-3-ol (110 mg, 48%) as an off-white solid. MS ESI calculated for C₁₁H₁₅ClN₄O₂[M+H]⁺, 271.09, found 271.05. ¹H NMR (400 MHz, DMSO-d₆) δ 6.15 (s, 1H), 5.64 (d, J=6.4 Hz, 1H), 4.51-4.44 (m, 1H), 4.15-4.11 (m, 2H), 3.70-3.66 (m, 2H), 3.61-3.59 (m, 4H), 3.52-3.50 (m, 4H).

### Example 15: (3S)-N-[3-[2-(3-hydroxyazetidin-1-yl)-6-(morpholin-4-yl)pyrimidin-4-yl]-4-methylphenyl]-3-(2,2,2-trifluoroethyl)pyrrolidine-1-carboxamide

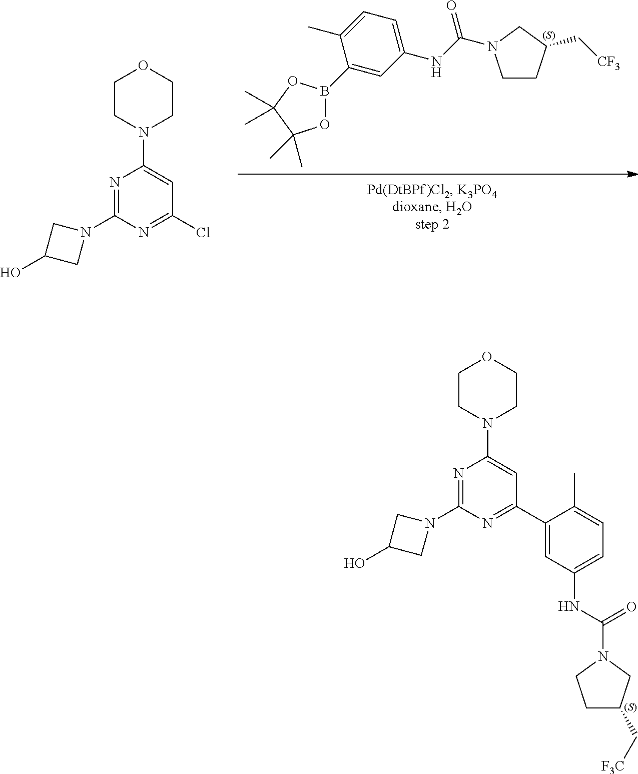

A mixture of 1-[4-chloro-6-(morpholin-4-yl)pyrimidin-2-yl]azetidin-3-ol (50 mg, 0.185 mmol), (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (76 mg, 0.185 mmol), Pd(DtBPF)Cl₂ (12 mg, 0.018 mmol) and K₃PO₄ (78 mg, 0.369 mmol) in dioxane (0.9 mL) and H₂O (0.2 mL) was stirred for 16 h at 80° C. under nitrogen atmosphere. The resulting mixture was concentrated under vacuum.

The residue was purified by silica gel column chromatography, eluted with PE/EtOAc/EtOH(4/3/1), the crude product (50 mg) was purified by Prep-HPLC with the following conditions: (Column: XBridge Prep C18 OBD Column, 19×150 mm 5 um; Mobile Phase A:Water(10 mmol/L NH₄HCO₃), Mobile Phase B: CH₃CN; Flow rate:20 mL/min; Gradient:40 B to 75 B in 4.3 min; 210/254 nm) to afford (3S)-N-[3-[2-(3-hydroxyazetidin-1-yl)-6-(morpholin-4-yl)pyrimidin-4-yl]-4-methylphenyl]-3-(2,2,2-trifluoroethyl)pyrrolidine-1-carboxamide (38 mg, 39%) as an off-white solid. MS ESI calculated for C₂₅H₃₁F₃N₆O₃[M+H]⁺, 521.24, found 521.25. ¹H NMR (400 MHz, DMSO-d₆) δ 8.15 (s, 1H), 7.48-7.45 (m, 2H), 7.10-7.08 (m, 1H), 6.10 (s, 1H), 5.60-5.58 (m, 1H), 4.52-4.50 (m, 1H), 4.18-4.14 (m, 2H), 3.83 (m, 2H), 3.74-3.64 (m, 5H), 3.56-3.51 (m, 5H), 3.31-3.30 (m, 1H), 3.05-3.00 (m, 1H), 2.51-2.41 (m, 3H), 2.24 (s, 3H), 2.08 (m, 1H), 1.75-1.55 (m, 1H). ¹⁹F NMR (376 MHz, DMSO-d₆) δ-63.36 (3F).

### Example 16: (3S)-N-[3-[2-(3-hydroxyazetidin-1-yl)-6-(morpholin-4-yl)pyridin-4-yl]-4-methylphenyl]-3-(2,2,2-trifluoroethyl)pyrrolidine-1-carboxamide

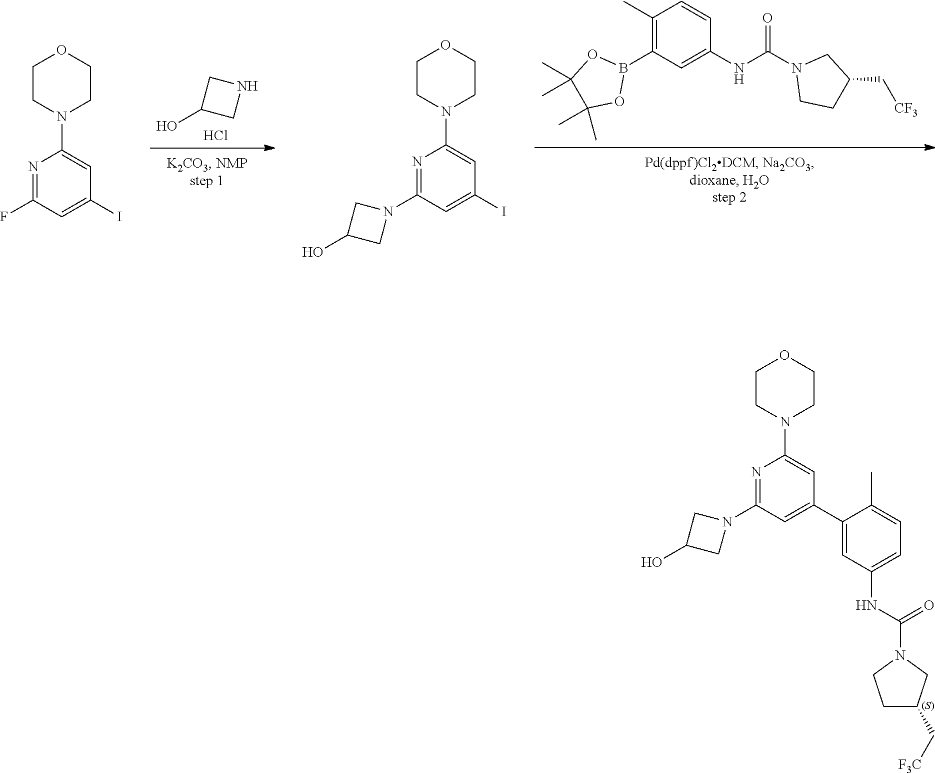

Preparation 16A: 1-[4-iodo-6-(morpholin-4-yl)pyridin-2-yl]azetidin-3-ol

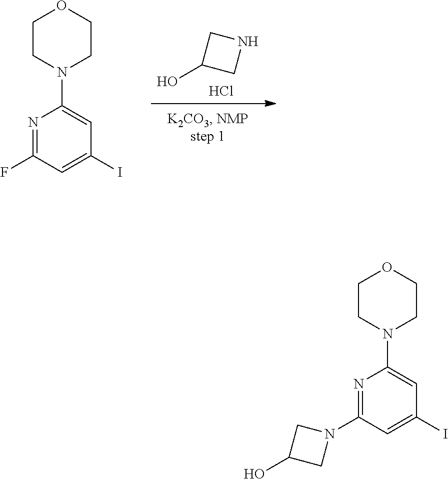 [SMILES: Fc1cc(I)cc(N2CCOCC2)n1 ; OC1CNC1 ; Cl ; OC1CN(c2cc(I)cc(N3CCOCC3)n2)C1]

To a stirred solution of 4-(6-fluoro-4-iodopyridin-2-yl)morpholine (300 mg, 0.974 mmol) and K₂CO₃ (269 mg, 1.947 mmol) in NMP (3 mL) was added azetidin-3-ol hydrochloride (160 mg, 1.461 mmol) at room temperature under nitrogen atmosphere. The resulting mixture was stirred for 16 h at 150° C. under nitrogen atmosphere. The reaction was quenched by the addition of water (100 mL). The resulting mixture was extracted with EtOAc (3×50 mL). The combined organic layers were washed with saturated brine (3×50 mL), dried over anhydrous Na₂SO₄. After filtration, the filtrate was concentrated under reduced pressure. The residue was purified by silica gel column chromatography, eluted with PE/EtOAc (2:1) to afford 1-[4-iodo-6-(morpholin-4-yl)pyridin-2-yl]azetidin-3-ol (90 mg, 26%) as an off-white solid. MS ESI calculated for C₁₂H₁₆IN₃O₂[M+H]⁺, 361.03, found 361.90. ¹H NMR (400 MHz, DMSO-d₆) δ 6.37 (m, 1H), 6.06-6.05 (m, 1H), 5.61-5.59 (m, 1H), 4.53-4.43 (m, 1H), 4.09-4.03 (m, 2H), 3.65-3.58 (m, 6H), 3.37-3.35 (m, 4H).

### Example 16: (3S)-N-[3-[2-(3-hydroxyazetidin-1-yl)-6-(morpholin-4-yl)pyridin-4-yl]-4-methylphenyl]-3-(2,2,2-trifluoroethyl)pyrrolidine-1-carboxamide

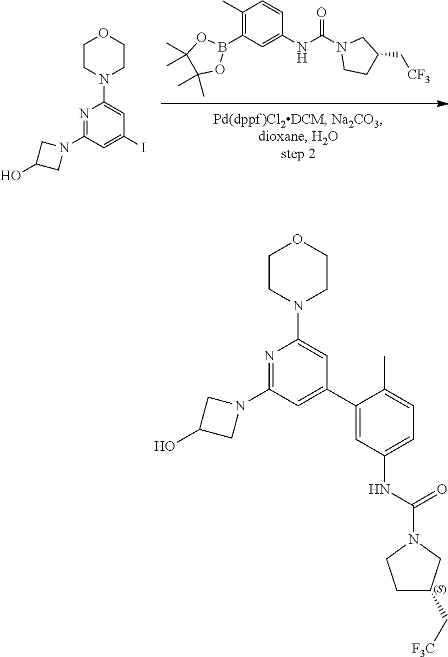

To a stirred solution of 1-[4-iodo-6-(morpholin-4-yl)pyridin-2-yl]azetidin-3-ol (50 mg, 0.138 mmol), (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (60 mg, 0.145 mmol) and Na₂CO₃ (44 mg, 0.415 mmol) in dioxane (0.8 mL) and H₂O (0.2 mL) was added Pd(dppf)Cl₂·CH₂Cl₂ (11 mg, 0.014 mmol) at room temperature under nitrogen atmosphere. The resulting mixture was stirred for 16 h at 60° C. under nitrogen atmosphere. The reaction was quenched by the addition of water (100 mL). The resulting mixture was extracted with EtOAc (3×50 mL). The combined organic layers were washed with saturated brine (3×50 mL), dried over anhydrous Na₂SO₄. After filtration, the filtrate was concentrated under reduced pressure. The residue was purified by silica gel column chromatography, eluted with PE/EA/EtOH (8/3/1) to afford 50 mg crude product. The residue was purified by reverse flash chromatography with the following conditions: Column: XBridge Prep C18 OBD Column, 30*100 mm, 5 um; Mobile Phase A:Water(10 mmol/l NH₄HCO₃), Mobile Phase B: CH₃CN; Flow rate:20 mL/min; Gradient:35 B to 65 B in 8 min; 210/254 nm. This resulted in (3S)-N-[3-[2-(3-hydroxyazetidin-1-yl)-6-(morpholin-4-yl)pyridin-4-yl]-4-methylphenyl]-3-(2,2,2-trifluoroethyl)pyrrolidine-1-carboxamide (33 mg, 46%) as an off-white solid. MS ESI calculated for C₂₆H₃₂F₃N₅O₃[M+H]⁺. 519.25, found 520.15. ¹H NMR (400 MHz, DMSO-d₆) δ 8.12 (s, 1H), 7.45-7.42 (m, 1H), 7.34-7.33 (m, 1H), 7.12-7.10 (m, 1H), 5.93 (s, 1H), 5.61-5.57 (m, 2H), 4.56-4.52 (m, 1H), 4.12-4.08 (m, 2H), 3.69-3.61 (m, 7H), 3.59-3.50 (m, 1H), 3.41-3.39 (m, 4H), 3.31-3.27 (m, 1H), 3.04-3.00 (m, 1H), 2.47-2.38 (m, 2H), 2.16 (s, 3H), 2.10-2.08 (m, 1H), 1.71-1.63 (m, 1H), 1.24 (m, 1H). ¹⁹F NMR (376 MHz, DMSO-d₆) δ-63.36 (3F).

### Example 17: (3R)-N-[3-(2-[[(2R)-1-hydroxypropan-2-yl]amino]-6-(morpholin-4-yl)pyridin-4-yl)-4-methylphenyl]-3-(trifluoromethoxy)pyrrolidine-1-carboxamide

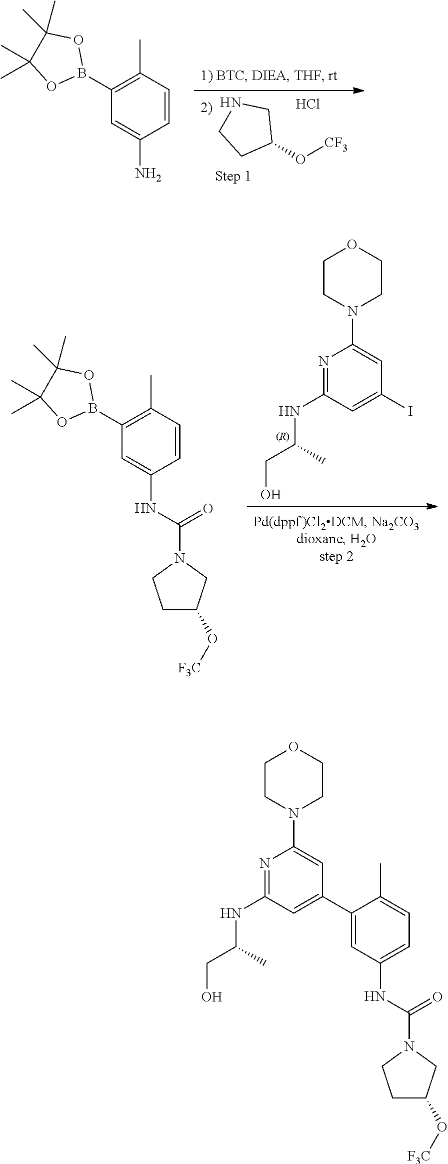

Preparation 17A: (3R)-N-[4-methyl-3-(4,4,5,5-tetramethyl-1,3,2-dioxaborolan-2-yl)phenyl]-3-(trifluoromethoxy)pyrrolidine-1-carboxamide

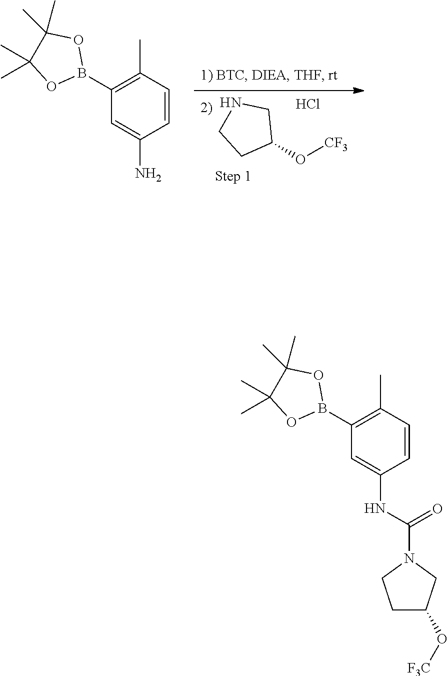 [SMILES: Cc1ccc(N)cc1B1OC(C)(C)C(C)(C)O1 ; FC(F)(F)O[C@@H]1CCNC1 ; Cl ; Cc1ccc(NC(=O)N2CC[C@@H](OC(F)(F)F)C2)cc1B1OC(C)(C)C(C)(C)O1]

To a stirred solution of (3R)-3-(trifluoromethoxy)pyrrolidine hydrochloride (150 mg, 0.64 mmol) and DIEA (415.80 mg, 3.22 mmol) in THF (5 mL) was added triphosgene (76 mg, 0.26 mmol,) at room temperature. The resulting mixture was stirred for 30 min at room temperature. To this was added (3R)-3-(trifluoromethoxy)pyrrolidine hydrochloride (136 mg, 0.708 mmol). The solution was stirred for 1 h at room temperature. The resulting mixture was concentrated under vacuum. The residue was purified by silica gel column chromatography, eluted with EA/PE (0-60%) to afford (3R)-N-[4-methyl-3-(4,4,5,5-tetramethyl-1,3,2-dioxaborolan-2-yl)phenyl]-3-(trifluoromethoxy)pyrrolidine-1-carboxamide (250 mg, 94%) as an off-white solid. MS ESI calculated for C₁₉H₂₆BF₃N₂O₄ [M+H]⁺, 415.19, found 415.25. ¹H NMR (400 MHz, Chloroform-d) δ 7.73-7.71 (m, 1H), 7.46-7.45 (m, 1H), 7.16-7.14 (m, 1H), 6.14 (s, 1H), 4.95-4.92 (m, 1H), 3.80-3.71 (m, 2H), 3.64-3.61 (m, 2H), 2.51 (s, 3H), 2.37-2.20 (m, 2H), 1.37 (s, 12H). ¹⁹F NMR (376 MHz, Chloroform-d) 6-58.70 (3F).

### Example 17: (3R)-N-[3-(2-[[(2R)-1-hydroxypropan-2-yl]amino]-6-(morpholin-4-yl)pyridin-4-yl)-4-methylphenyl]-3-(trifluoromethoxy)pyrrolidine-1-carboxamide

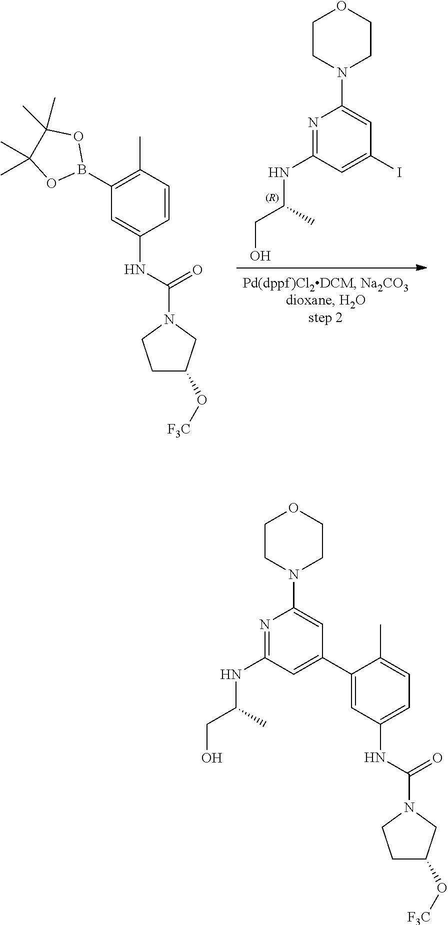

To a stirred solution of (2R)-2-[[4-iodo-6-(morpholin-4-yl)pyridin-2-yl]amino]propan-1-ol (109 mg, 0.300 mmol) in dioxane (2 mL) and H₂O (0.2 mL) were added (3R)-N-[4-methyl-3-(4,4,5,5-tetramethyl-1,3,2-dioxaborolan-2-yl)phenyl]-3-(trifluoromethoxy)pyrrolidine-1-carboxamide (149 mg, 0.360 mmol), Na₂CO₃ (191 mg, 0.900 mmol) and Pd(dppf)Cl₂CH₂Cl₂ (25 mg, 0.030 mmol) at room temperature under nitrogen atmosphere. The resulting mixture was stirred for 1 h at 80° C. under nitrogen atmosphere. The resulting mixture was filtered, the filter cake was washed with EtOAc (3×10 mL). The filtrate was concentrated under reduced pressure. The residue was purified by silica gel column chromatography, eluted with EtOAc in PE (0-82%) to afford crude product as a brown solid. The crude product (201 mg) was purified by Prep-HPLC with the following conditions: Column: Atlantis Prep T3 OBD Column, 19*250 mm 10u; Mobile Phase A:Water(0.1% FA), Mobile Phase B: CH₃CN; Flow rate:20 mL/min; Gradient:25 B to 50 B in 6 min; 210/254 nm to afford (3R)-N-[3-(2-[[(2R)-1-hydroxypropan-2-yl]amino]-6-(morpholin-4-yl)pyridin-4-yl)-4-methylphenyl]-3-(trifluoromethoxy)pyrrolidine-1-carboxamide (83 mg, 53%) as an off-white solid. MS ESI calculated for C₂₅H₃₃F₃N₅O₄[M+H]⁺, 524.25, 526.25, found 524.10.526.10. ¹H NMR (300 MHz, DMSO-d₆) δ 8.24 (s, 1H), 7.47-7.34 (m, 2H), 7.12 (d, J=8.4 Hz, 1H), 5.98 (d, J=7.7 Hz, 1H), 5.77 (s, 2H), 5.16 (s, 1H), 4.65 (s, 1H), 3.90 (t, J=6.5 Hz, 1H), 3.82-3.59 (m, 7H), 3.55-3.37 (m, 6H), 2.18 (s, 6H), 1.14 (d, J=6.6 Hz, 3H).

### Example 18: N-[3-(2-[[(2R)-1-hydroxypropan-2-yl]amino]-6-(morpholin-4-yl)pyridin-4-yl)-4-methylphenyl]-3-(2,2,2-trifluoroethyl)-2,5-dihydropyrrole-1-carboxamide

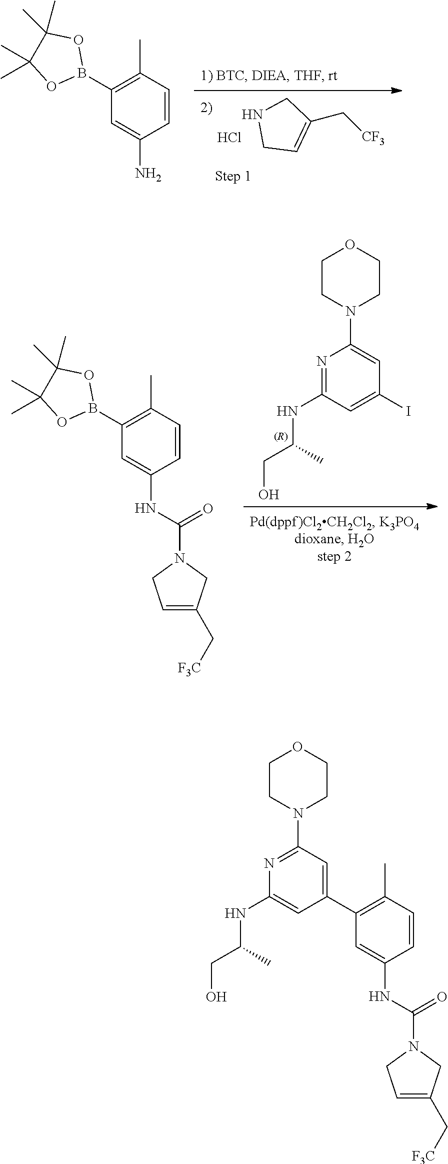

Preparation 18A: N-[4-methyl-3-(4,4,5,5-tetramethyl-1,3,2-dioxaborolan-2-yl)phenyl]-3-(2,2,2-trifluoroethyl)-2,5-dihydropyrrole-1-carboxamide

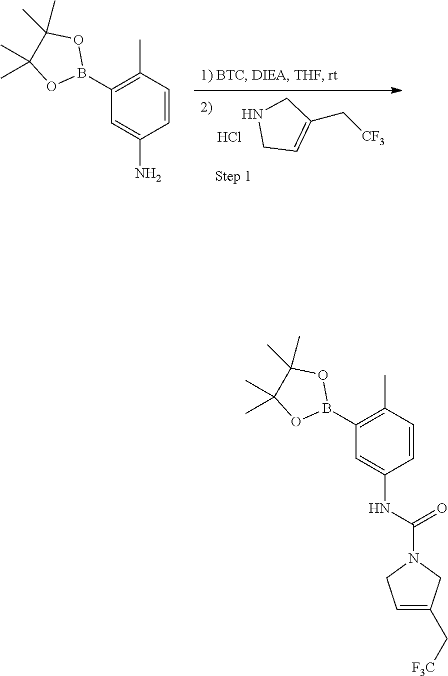 [SMILES: Cc1ccc(N)cc1B1OC(C)(C)C(C)(C)O1 ; Cl ; FC(F)(F)CC1=CCNC1 ; Cc1ccc(NC(=O)N2CC=C(CC(F)(F)F)C2)cc1B1OC(C)(C)C(C)(C)O1]

To a stirred mixture of 4-methyl-3-(4,4,5,5-tetramethyl-1,3,2-dioxaborolan-2-yl)aniline (68 mg, 0.292 mmol, 1.00 equiv) in THE (4 mL) were added DIEA (188 mg, 1.458 mmol), Triphosgene (35 mg, 0.117 mmol) at room temperature. The resulting mixture was stirred for 0.5 h at room temperature. To this was added 3-(2,2,2-trifluoroethyl)-2,5-dihydro-1H-pyrrole hydrochloride (60 mg, 0.321 mmol) in THE (3 mL) was dropwised at room temperature. The resulting mixture was stirred for 2 h at room temperature. The resulting mixture was diluted with MeOH (10 mL) and concentrated under vacuum. The residue was purified by silica gel column chromatography, eluted with hexane/(EtOAc:EtOH=3:1) (1:1) to afford N-[4-methyl-3-(4,4,5,5-tetramethyl-1,3,2-dioxaborolan-2-yl)phenyl]-3-(2,2,2-trifluoroethyl)-2,5-dihydropyrrole-1-carboxamide (100 mg, 84%) as a light brown oil. MS ESI calculated for C₂₀H₂₆BF₃N₂O₃ [M+H]⁺, 411.20, found 411.20. ¹H NMR (300 MHz, DMSO-d₆) δ 8.22 (s, 1H), 7.69 (d, J=2.4 Hz, 1H), 7.64 (d, J=8.4 Hz, 1H), 7.06 (d, J=8.4 Hz, 1H), 5.95 (s, 1H), 4.21 (s, 4H), 3.32 (q, J=11.7 Hz, 2H), 2.40 (s, 3H), 1.31 (s, 12H).

### Example 18: N-[3-(2-[[(2R)-1-hydroxypropan-2-yl]amino]-6-(morpholin-4-yl)pyridin-4-yl)-4-methylphenyl]-3-(2,2,2-trifluoroethyl)-2,5-dihydropyrrole-1-carboxamide

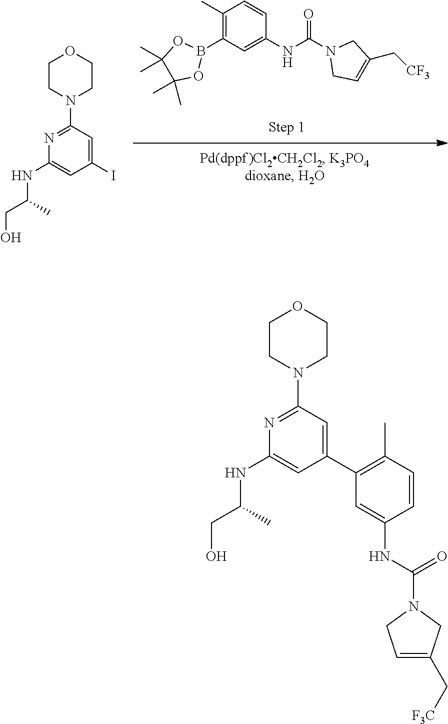 [SMILES: C[C@H](CO)Nc1cc(I)cc(N2CCOCC2)n1 ; Cc1ccc(NC(=O)N2CC=C(CC(F)(F)F)C2)cc1B1OC(C)(C)C(C)(C)O1 ; Cc1ccc(NC(=O)N2CC=C(CC(F)(F)F)C2)cc1-c1cc(N[C@H](C)CO)nc(N2CCOCC2)c1]

To a solution of (2R)-2-[[4-iodo-6-(morpholin-4-yl)pyridin-2-yl]amino]propan-1-ol (132 mg, 0.36 mmol) and N-[4-methyl-3-(4,4,5,5-tetramethyl-1,3,2-dioxaborolan-2-yl)phenyl]-3-(2,2,2-trifluoroethyl)-2,5-dihydropyrrole-1-carboxamide (149 mg, 0.363 mmol) in 1,4-dioxane (2 mL) and H₂O (0.2 mL) were added Pd(dppf)Cl₂·CH₂Cl₂ (30 mg, 0.030 mmol) and K₃PO₄ (231 mg, 1.090 mmol). After stirring for 1 h at 80° C. under a nitrogen atmosphere, the resulting mixture was concentrated under reduced pressure. The residue was purified by silica gel column chromatography, eluted with EtOAc in PE (0-50%) to afford the crude product. The crude product was purified by Prep-HPLC with the following conditions: Column: Spherical C18, 19×150 mm 5 um; Mobile Phase A: Water (0.1% FA), Mobile Phase B: CH₃CN: Flow rate: 20 mL/min; Gradient: 30% B to 60% B in 4.3 min; Detector: UV 210 & 254 nm to afford N-[3-(2-[[(2R)-1-hydroxypropan-2-yl]amino]-6-(morpholin-4-yl)pyridin-4-yl)-4-methylphenyl]-3-(2,2,2-trifluoroethyl)-2,5-dihydropyrrole-1-carboxamide (82 mg, 44%) as a light pink solid. MS ESI calculated for C₂₆H₃₄F₃N₅O₃[M+H]⁺,520.25, found 520.10. ¹H NMR (400 MHz, DMSO-d₆) δ 8.19 (s, 1H), 7.43 (dd, J=8.2, 2.3 Hz, 1H), 7.35 (d, J=2.3 Hz, 1H), 7.11 (d, J=8.3 Hz, 1H), 6.00-5.91 (m, 2H), 5.76 (d, J=1.6 Hz, 2H), 4.64 (t, J=5.5 Hz, 1H), 4.18 (S, 4H) 3.89 (p, J=6.5 Hz, 1H), 3.72-3.64 (m, 4H), 3.50 (dt, J=10.0, 5.0 Hz, 1H), 3.37 (t, J=4.9 Hz, 4H), 3.32-3.25 (m, 2H), 2.17 (s, 3H), 1.13 (d, J=6.5 Hz, 3H). ¹⁹F NMR (376 MHz, DMSO-d₆) δ-63.28 (3F)

### Example 19: (3S)-N-(3-[2-[(2-hydroxyethyl)amino]-6-(morpholin-4-yl)pyridin-4-yl]-4-methylphenyl)-3-(2,2,2-trifluoroethyl)pyrrolidine-1-carboxamide

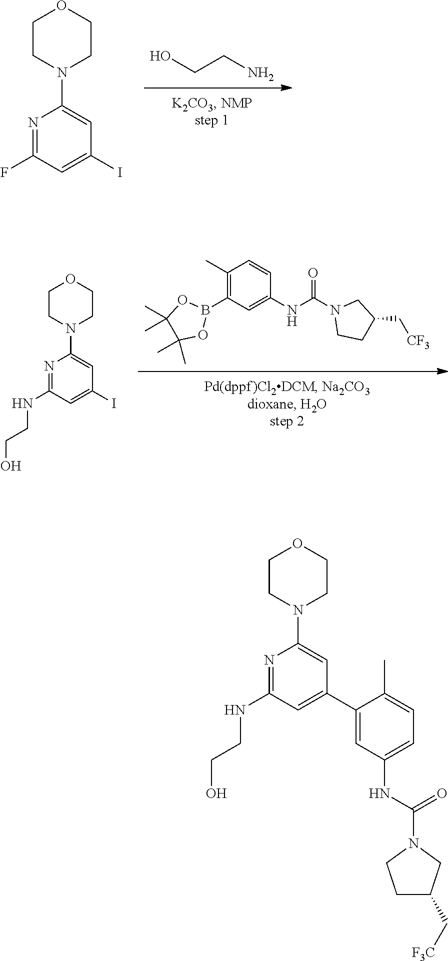

Preparation 19A: 2-[[4-iodo-6-(morpholin-4-yl)pyridin-2-yl]amino]ethanol

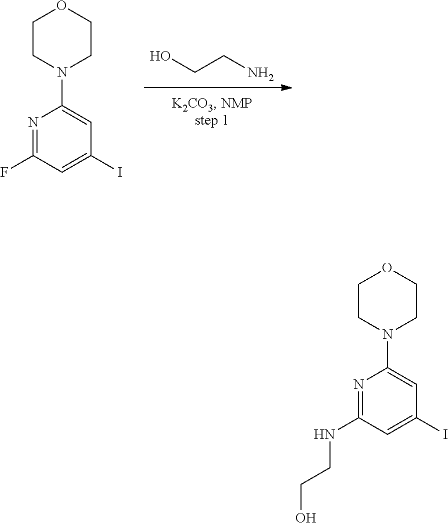

To a stirred mixture of 4-(6-fluoro-4-iodopyridin-2-yl)morpholine (2.00 g, 6.492 mmol) and K₂CO₃ (1.79 g, 12.983 mmol) in NMP (20 mL) was added ethanolamine(595 mg, 9.737 mmol) at room temperature. The resulting mixture was stirred for 16 h at 150° C. The mixture was allowed to cool down to room temperature. The reaction was quenched with Water (20 mL) at room temperature. The resulting mixture was extracted with EtOAc (3×20 mL). The combined organic layers were washed with brine (1×20 mL), dried over anhydrous Na₂SO₄. After filtration, the filtrate was concentrated under reduced pressure. The residue was purified by silica gel column chromatography, eluted with PE/EtOAc (1:1) to afford 2-[[4-iodo-6-(morpholin-4-yl)pyridin-2-yl]amino]ethanol (2 g, 88%) as a yellow oil. MS ESI calculated for C₁₅H₂₀BrN₃O₂[M+H]⁺ 350.03 found 350.00. ¹H NMR (300 MHz, DMSO-d₆) δ 6.23 (m, 2H), 4.64 (m, 1H), 3.66 (t, J=4.6 Hz, 4H), 2.72 (d, J=1.2 Hz, 8H).

### Example 19:(3S)-N-(3-[2-[(2-hydroxyethyl)amino]-6-(morpholin-4-yl)pyridin-4-yl]-4-methylphenyl)-3-(2,2,2-trifluoroethyl)pyrrolidine-1-carboxamide

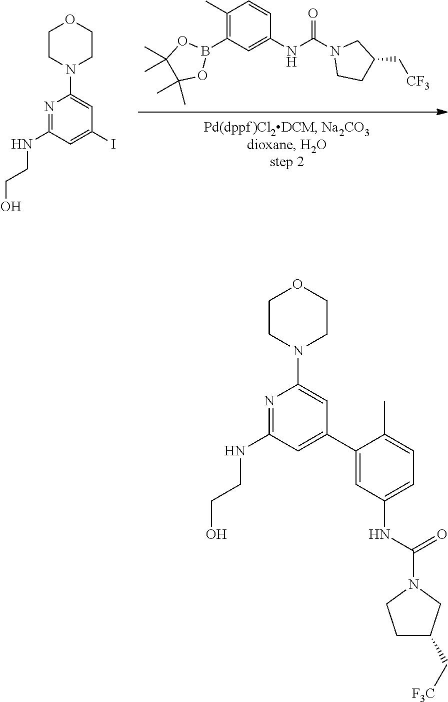 [SMILES: OCCNc1cc(I)cc(N2CCOCC2)n1 ; Cc1ccc(NC(=O)N2CC[C@@H](CC(F)(F)F)C2)cc1B1OC(C)(C)C(C)(C)O1 ; Cc1ccc(NC(=O)N2CC[C@@H](CC(F)(F)F)C2)cc1-c1cc(NCCO)nc(N2CCOCC2)c1]

To a stirred mixture of 2-[[4-iodo-6-(morpholin-4-yl)pyridin-2-yl]amino]ethanol (150 mg, 0.430 mmol) and (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (142 mg, 0.344 mmol) in dioxane (4 mL) and H₂O (1 mL) were added Na₂CO₃ (137 mg, 1.289 mmol) and Pd(dppf)Cl₂ CH₂Cl₂ (35 mg, 0.043 mmol) in portions at 60° C. under nitrogen atmosphere. The reaction was quenched with (20 mL) Water at room temperature. The resulting mixture was extracted with EtOAc (3×60 mL). The combined organic layers were washed with brine (3×10 mL), dried over anhydrous Na₂SO₄. After filtration, the filtrate was concentrated under reduced pressure. The residue was purified by silica gel column chromatography, eluted with PE/EtOAc (1:1) to afford (3S)-N-(3-[2-[(2-hydroxyethyl)amino]-6-(morpholin-4-yl)pyridin-4-yl]-4-methylphenyl)-3-(2,2,2-trifluoroethyl)pyrrolidine-1-carboxamide (95 mg, 43%) as a light green solid. MS ESI calculated for C₁₅H₂₀BrN₃O₂[M+H]⁺ 508.25 found 508.00. ¹H NMR (400 MHz, DMSO-d₆) δ 8.11 (s, 1H), 7.49-7.27 (m, 2H), 7.09 (d, J=8.4 Hz, 1H), 6.21 (s, 1H), 5.76 (s, 2H), 4.66 (t, J=5.5 Hz, 1H), 3.68 (s, 3H), 3.53 (d, J=7.4 Hz, 3H), 3.38 (s, 9H), 3.02 (t, J=9.4 Hz, 1H), 2.44 (s, 3H), 2.12 (d, J=29.5 Hz, 4H), 1.66 (t, J=10.6 Hz, 1H).

### Example 20: N-(3-[2-[(2-hydroxyethyl)amino]-6-(morpholin-4-yl)pyridin-4-yl]-4-methylphenyl)-3-(2,2,2-trifluoroethyl)-2,5-dihydropyrrole-1-carboxamide

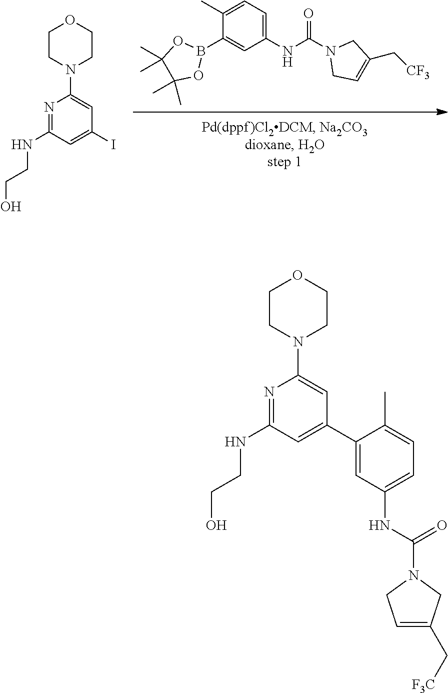 [SMILES: OCCNc1cc(I)cc(N2CCOCC2)n1 ; Cc1ccc(NC(=O)N2CC=C(CC(F)(F)F)C2)cc1B1OC(C)(C)C(C)(C)O1 ; Cc1ccc(NC(=O)N2CC=C(CC(F)(F)F)C2)cc1-c1cc(NCCO)nc(N2CCOCC2)c1]

To a stirred mixture of 2-[[4-iodo-6-(morpholin-4-yl)pyridin-2-yl]amino]ethanol (150 mg, 0.430 mmol) and N-[4-methyl-3-(4,4,5,5-tetramethyl-1,3,2-dioxaborolan-2-yl)phenyl]-3-(2,2,2-trifluoroethyl)-2,5-dihydropyrrole-1-carboxamide (141 mg, 0.344 mmol) in dioxane (4 mL) and H₂O (1 mL) were added Na₂CO₃ (137 mg, 1.289 mmol) and Pd(dppf)Cl₂ CH₂Cl₂ (35 mg, 0.043 mmol) in portions at 60° C. under nitrogen atmosphere. The reaction was quenched by the addition of Water (20 mL) at room temperature. The resulting mixture was extracted with EtOAc (3×60 mL). The combined organic layers were washed with brine (3×10 mL), dried over anhydrous Na₂SO₄. After filtration, the filtrate was concentrated under reduced pressure. The residue was purified by silica gel column chromatography, eluted with PE/EtOAc (1:1) to afford N-(3-[2-[(2-hydroxyethyl)amino]-6-(morpholin-4-yl)pyridin-4-yl]-4-methylphenyl)-3-(2,2,2-trifluoroethyl)-2,5-dihydropyrrole-1-carboxamide (97 mg, 44%) as a light green solid. MS ESI calculated for C₂₅H₃₀F₃N₅O₃ [M+H]⁺ 506.23 found 506.00. ¹H NMR (400 MHz, DMSO-d₆) δ 8.20 (s, 1H), 7.49-7.30 (m, 2H), 7.11 (d, J=8.4 Hz, 1H), 6.23 (s, 1H), 5.94 (s, 1H), 5.77 (s, 2H), 4.66 (s, 1H), 4.20 (s, 4H), 3.68 (s, 4H), 3.54 (s, 2H), 3.36 (d, J=18.2 Hz, 8H), 2.17 (s, 3H).

### Example 21: (3S)-N-[3-[2-(3-hydroxyazetidin-1-yl)-6-(morpholin-4-yl)pyrimidin-4-yl]-4-methylphenyl]-3-(2,2,2-trifluoroethyl)pyrrolidine-1-carboxamide

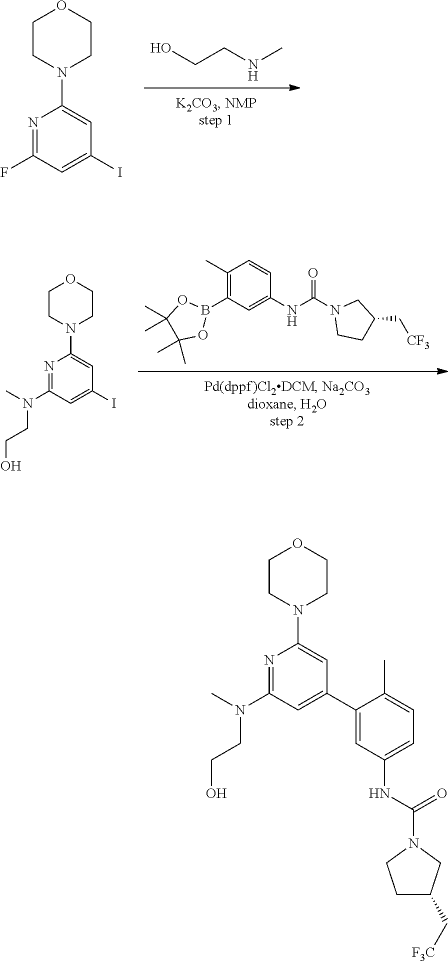

Preparation 21A: 2-[[4-iodo-6-(morpholin-4-yl)pyridin-2-yl](methyl)amino]ethanol

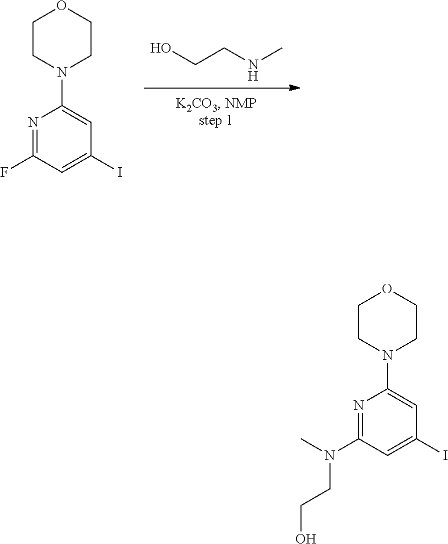

To a stirred mixture of 4-(6-fluoro-4-iodopyridin-2-yl)morpholine(500 mg, 1.623 mmol) and K₂CO₃ (449 mg, 3.246 mmol) in NMP (5 mL) was added methylethanolamine (183 mg, 2.434 mmol) at room temperature. The resulting mixture was stirred for 16 h at 150° C. The mixture was allowed to cool down to room temperature. The reaction was quenched with water (20 mL) at room temperature. The resulting mixture was extracted with EtOAc (3×20 mL). The combined organic layers were washed with brine (20 mL), dried over anhydrous Na₂SO₄. After filtration, the filtrate was concentrated under reduced pressure. The residue was purified by silica gel column chromatography, eluted with PE/EtOAc (1/1) to afford 2-[[4-iodo-6-(morpholin-4-yl)pyridin-2-yl](methyl)amino]ethanol(480 mg, 81%) as a yellow oil. MS ESI calculated for C₁₂H₁₈IN₃O₂[M+H]⁺, 363.95, found 363.95. ¹H NMR (300 MHz, chloroform-d) δ 6.34-6.26 (m, 2H), 3.80 (m, 6H), 3.70 (dd, J=5.5, 4.4 Hz, 2H), 3.47-3.36 (m, 4H), 3.02 (s, 3H).

### Example 21: (3S)-N-[3-[2-(3-hydroxyazetidin-1-yl)-6-(morpholin-4-yl)pyrimidin-4-yl]-4-methylphenyl]-3-(2,2,2-trifluoroethyl)pyrrolidine-1-carboxamide

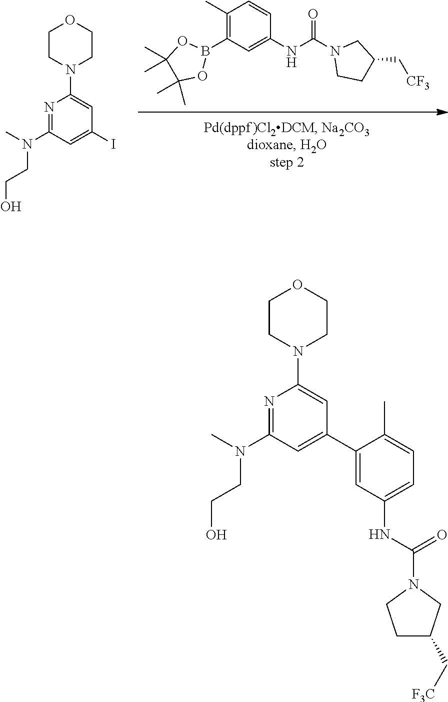 [SMILES: CN(CCO)c1cc(I)cc(N2CCOCC2)n1 ; Cc1ccc(NC(=O)N2CC[C@@H](CC(F)(F)F)C2)cc1B1OC(C)(C)C(C)(C)O1 ; Cc1ccc(NC(=O)N2CC[C@@H](CC(F)(F)F)C2)cc1-c1cc(N(C)CCO)nc(N2CCOCC2)c1]

To a stirred solution of 2-[[4-iodo-6-(morpholin-4-yl)pyridin-2-yl](methyl)amino]ethanol (200 mg, 0.551 mmol) and (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (227 mg, 0.551 mmol) in 1,4-dioxane (2 mL) and H₂O (0.5 mL) were added Na₂CO₃ (175 mg, 1.652 mmol) and Pd(dppf)Cl₂ CH₂Cl₂ (40 mg, 0.055 mmol) at room temperature under nitrogen atmosphere. The resulting mixture was stirred for 16 h at 70° C. under nitrogen atmosphere. The mixture was allowed to cool down to room temperature. The reaction was quenched by the addition of Water (10 mL) at room temperature. The resulting mixture was extracted with EtOAc (3×10 mL). The combined organic layers were washed with brine (10 mL), dried over anhydrous Na₂SO₄. After filtration, the filtrate was concentrated under reduced pressure. The residue was purified by silica gel column chromatography, eluted with PE/EtOAc/EtOH (4/3/1) to afford (3S)-N-(3-[2-[(2-hydroxyethyl)(methyl)amino]-6-(morpholin-4-yl)pyridin-4-yl]-4-methylphenyl)-3-(2,2,2-trifluoroethyl)pyrrolidine-1-carboxamide (172 mg, 60%) as a light brown solid. MS ESI calculated for C₂₆H₃₄F₃N₅O₃[M+H]⁺, 522.15, found 522.15. ¹H NMR (400 MHz, DMSO-d₆) δ 8.12 (s, 1H), 7.44 (dd, J=8.3, 2.3 Hz, 1H), 7.34 (d, J=2.3 Hz, 1H), 7.11 (d, J=8.4 Hz, 1H), 5.84 (d, J=13.6 Hz, 2H), 4.68-4.59 (m, 1H), 3.69 (d, J=5.2 Hz, 5H), 3.61-3.48 (m, 5H), 3.40 (t, J=4.7 Hz, 4H), 3.03 (s, 4H), 2.44 (t, J=10.4 Hz, 3H), 2.17 (s, 3H), 2.09 (s, 1H), 2.01 (s, 1H), 1.73-1.60 (m, 1H). ¹⁹F NMR (376 MHz, DMSO-d₆) δ-63.36 (3F).

### Example 22: (3R)-N-(3-[2-[(2-hydroxyethyl)amino]-6-(morpholin-4-yl)pyridin-4-yl]-4-methylphenyl)-3-(trifluoromethoxy)pyrrolidine-1-carboxamide

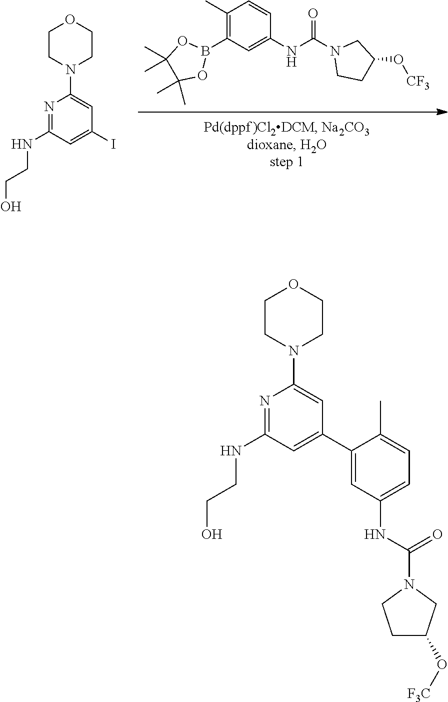 [SMILES: OCCNc1cc(I)cc(N2CCOCC2)n1 ; Cc1ccc(NC(=O)N2CC[C@@H](OC(F)(F)F)C2)cc1B1OC(C)(C)C(C)(C)O1 ; Cc1ccc(NC(=O)N2CC[C@@H](OC(F)(F)F)C2)cc1-c1cc(NCCO)nc(N2CCOCC2)c1]

To a stirred mixture of 2-[[4-iodo-6-(morpholin-4-yl)pyridin-2-yl]amino]ethanol (150 mg, 0.430 mmol) and (3R)-N-[4-methyl-3-(4,4,5,5-tetramethyl-1,3,2-dioxaborolan-2-yl)phenyl]-3-(trifluoromethoxy)pyrrolidine-1-carboxamide (142 mg, 0.344 mmol) in dioxane (4 ml) and H₂O (1 mL) were added Na₂CO₃ (137 mg, 1.289 mmol) and Pd(dppf)Cl₂ CH₂Cl₂ (35 mg, 0.043 mmol) in portions at 60° C. under nitrogen atmosphere. The reaction was quenched by the addition of Water (20 mL) at room temperature. The resulting mixture was extracted with EtOAc (3×60 mL). The combined organic layers were washed with brine (3×10 mL), dried over anhydrous Na₂SO₄. After filtration, the filtrate was concentrated under reduced pressure. The residue was purified by silica gel column chromatography, eluted with PE/EtOAc (1/1) to afford (3R)-N-(3-[2-[(2-hydroxyethyl)amino]-6-(morpholin-4-yl)pyridin-4-yl]-4-methylphenyl)-3-(trifluoromethoxy)pyrrolidine-1-carboxamide (74.3 mg, 34%) as a light green solid. MS ESI calculated for C₂₄H₃₀F₃N₅O₄ [M+H]⁺, 510.22 found 510.00. ¹H NMR (400 MHz, DMSO-d₆) δ 8.24 (s, 1H), 7.53-7.24 (m, 2H), 7.11 (d, J=8.3 Hz, 1H), 6.22 (t, J=5.9 Hz, 1H), 5.77 (s, 2H), 5.15 (s, 1H), 4.66 (t, J=5.5 Hz, 1H), 3.78-3.62 (m, 5H), 3.56 (dd, J=14.2, 8.1 Hz, 3H), 3.46 (d, J=8.9 Hz, 1H), 3.38 (s, 3H), 3.34 (s, 3H), 3.31 (d, J=5.9 Hz, 1H), 2.20-2.00 (m, 5H).

### Example 23: (3R)-N-[3-(2-[[(2R)-2-hydroxypropyl]amino]-6-(morpholin-4-yl)pyridin-4-yl)-4-methylphenyl]-3-(trifluoromethoxy)pyrrolidine-1-carboxamide

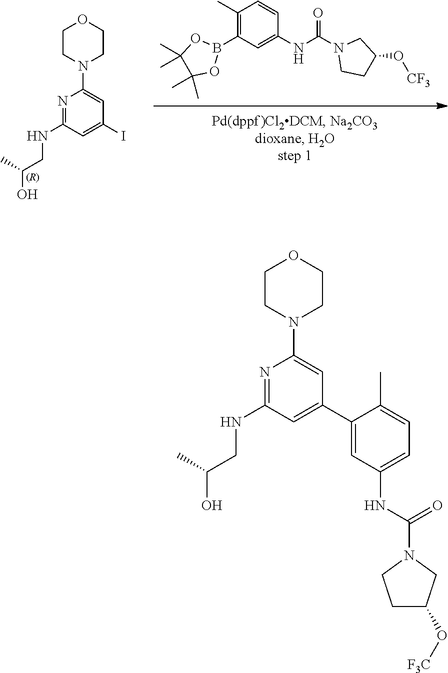

To a stirred solution of (2R)-1-[[4-iodo-6-(morpholin-4-yl)pyridin-2-yl]amino]propan-2-ol (132 mg, 0.363 mmol), (3R)-N-[4-methyl-3-(4,4,5,5-tetramethyl-1,3,2-dioxaborolan-2-yl)phenyl]-3-(trifluoromethoxy)pyrrolidine-1-carboxamide (151 mg, 0.363 mmol) and Na₂CO₃ (116 mg, 1.090 mmol) in dioxane (0.8 mL) and water (0.2 mL) was added Pd(dppf)Cl₂·CH₂Cl₂ (30 mg, 0.036 mmol) at room temperature under nitrogen atmosphere. The resulting mixture was stirred for 16 h at 60° C. under nitrogen atmosphere. The reaction was quenched by the addition of water (100 mL). The resulting mixture was extracted with EtOAc (3×50 mL). The combined organic layers were washed with saturated brine (3×50 mL), dried over anhydrous Na₂SO₄. After filtration, the filtrate was concentrated under reduced pressure. The residue was purified by silica gel column chromatography, eluted with PE/EA/EtOH (8/3/1) to afford 125 mg crude product. The residue was purified by reverse flash chromatography with the following conditions: Column: XBridge Prep C18 OBD Column, 19×150 mm Sum; Mobile Phase A: water (10 mmol/1 NH₄HCO₃), Mobile Phase B: CH₃CN; Flow rate:20 mL/min; Gradient:40 B to 75 B in 4.3 min; 210/254 nm. This resulted in (3R)-N-[3-(2-[[(2R)-2-hydroxypropyl]amino]-6-(morpholin-4-yl)pyridin-4-yl)-4-methylphenyl]-3-(trifluoromethoxy)pyrrolidine-1-carboxamide (102 mg, 54%) as a light green solid. MS ESI calculated for C₂₅H₃₂F₃N₅O₄[M+H]⁺, 524.24, found 524.15. ¹H NMR (400 MHz, DMSO-d₆) δ 8.22 (s, 1H), 7.42-7.33 (m, 2H), 7.12-7.09 (m, 1H), 6.20-6.15 (m, 1H), 5.83-5.76 (m, 2H), 5.14 (s, 1H), 4.69-4.67 (m, 1H), 3.83-3.76 (m, 1H), 3.67-3.55 (m, 7H), 3.48-3.37 (m, 5H), 3.20-3.14 (m, 2H), 2.24-2.16 (m, 5H), 1.09-1.07 (m, 3H). ¹⁹F NMR (376 MHz, DMSO-d₆)δ-56.69 (3F).

### Example 24: N-[3-(2-[[(2R)-2-hydroxypropyl]amino]-6-(morpholin-4-yl)pyridin-4-yl)-4-methylphenyl]-3-(2,2,2-trifluoroethyl)-2,5-dihydropyrrole-1-carboxamide

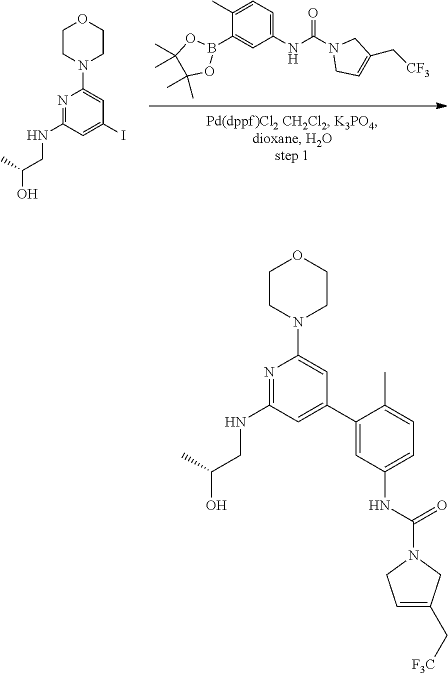 [SMILES: C[C@@H](O)CNc1cc(I)cc(N2CCOCC2)n1 ; Cc1ccc(NC(=O)N2CC=C(CC(F)(F)F)C2)cc1B1OC(C)(C)C(C)(C)O1 ; Cc1ccc(NC(=O)N2CC=C(CC(F)(F)F)C2)cc1-c1cc(NC[C@@H](C)O)nc(N2CCOCC2)c1]

A mixture of (2R)-1-[[4-iodo-6-(morpholin-4-yl)pyridin-2-yl]amino]propan-2-ol (133 mg, 0.366 mmol), N-[4-methyl-3-(4,4,5,5-tetramethyl-1,3,2-dioxaborolan-2-yl)phenyl]-3-(2,2,2-trifluoroethyl)-2,5-dihydropyrrole-1-carboxamid (150 mg, 0.370 mmol), Pd(dppf)Cl₂·CH₂Cl₂ (30 mg, 0.040 mmol) and K₃PO₄ (233 mg, 1.100 mmol) in dioxane (3 mL) and H₂O (0.3 mL) was stirred for 2 hour at 80° C. under N₂ atmosphere. The mixture was cooled. The resulting mixture was added water (5 mL) and extracted with EtOAc (3×10 mL). The combined organic layers were washed with water (2×10 mL), dried over anhydrous Na₂SO₄. After filtration, the filtrate was concentrated under reduced pressure. The residue was purified by silica gel column chromatography, eluted with PE/EtOAc (50-100%) to afford the crude product. The residue was purified by Prep-HPLC with the following conditions: Column: X-Bridge Prep C18 OBD Column, 19×150 mm 5 um; mobile phase A:water (10 mmol/L NH₄HCO₃), mobile phase B: ACN; Flow rate: 20 mL/min; Gradient: 20 B to 55 B in 4.3 min; 210/254 nm to afford N-[3-(2-[[(2R)-2-hydroxypropyl]amino]-6-(morpholin-4-yl)pyridin-4-yl)-4-methylphenyl]-3-(2,2,2-trifluoroethyl)-2,5-dihydropyrrole-1-carboxamide (97 mg, 51%) as a white solid. MS ESI calculated for C₁₉H₂₅BrN₄O₃[M+H]⁺, 437.11, found 437.00. ¹H NMR (400 MHz, CDCL₃) δ 7.37 (dd, J=8.2, 2.4 Hz, 1H), 7.24 (d, J=2.4 Hz, 1H), 7.18 (d, J=8.2 Hz, 1H), 6.13 (s, 1H), 5.88-5.82 (m, 3H), 4.70 (brs, 1H), 4.32-4.30 (m, 4H), 4.06-3.98 (m, 1H), 3.84-3.81 (m, 4H), 3.54-3.43 (m, 5H), 3.31-3.28 (m, 1H), 3.05-2.98 (m, 2H), 2.24 (s, 3H), 1.25 (d, J=6.3 Hz, 3H).

### Example 25: (3S)-N-[3-(2-[[(2R)-2-hydroxypropyl](methyl)amino]-6-(morpholin-4-yl)pyridin-4-yl)-4-methylphenyl]-3-(2,2,2-trifluoroethyl)pyrrolidine-1-carboxamide

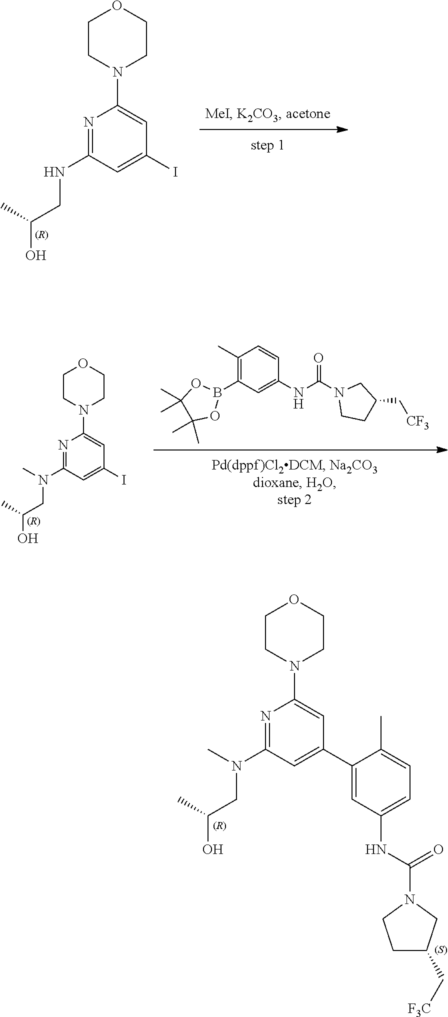

Preparation 25A: (2R)-1-[[4-iodo-6-(morpholin-4-yl)pyridin-2-yl](methyl)amino]propan-2-ol

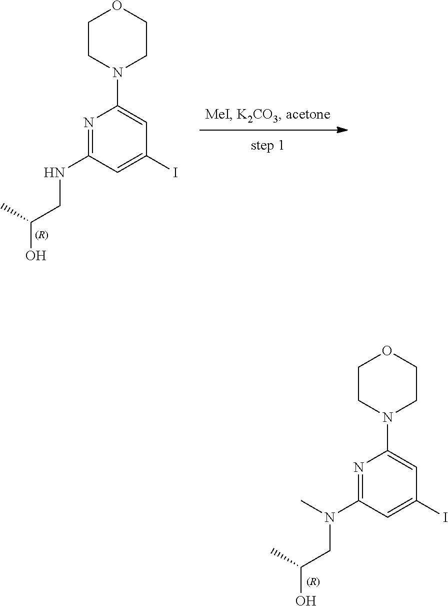

To a stirred solution of (2R)-1-[[4-iodo-6-(morpholin-4-yl)pyridin-2-yl]amino]propan-2-ol (500 mg, 1.377 mmol) and K₂CO₃ (571 mg, 4.130 mmol) in acetone (10 mL) was added methyl iodide (234 mg, 1.652 mmol) at 0° C. under nitrogen atmosphere. The resulting mixture was stirred for 48 h at 60° C. under nitrogen atmosphere. The reaction was quenched by the addition of water (100 mL). The resulting mixture was extracted with EtOAc (3×50 mL). The combined organic layers were washed with saturated brine (3×50 mL), dried over anhydrous Na₂SO₄. After filtration, the filtrate was concentrated under reduced pressure. The residue was purified by silica gel column chromatography, eluted with PE/EtOAc (2/1) to afford (2R)-1-[[4-iodo-6-(morpholin-4-yl)pyridin-2-yl](methyl)amino]propan-2-ol (150 mg, 29%) as an off-white solid. MS ESI calculated for C₁₃H₂₀IN₃O₂[M+H]⁺, 378.06, found 378.10. ¹H NMR (400 MHz, CDCL₃) δ 6.33-6.30 (m, 2H), 4.16-4.09 (m, 1H), 3.81-3.79 (m, 4H), 1.66-1.61 (m, 1H), 3.48-3.42 (m, 5H), 3.04 (s, 3H), 1.23-1.22 (m, 3H).

### Example 25: (3S)-N-[3-(2-[[(2R)-2-hydroxypropyl](methyl)amino]-6-(morpholin-4-yl)pyridin-4-yl)-4-methylphenyl]-3-(2,2,2-trifluoroethyl)pyrrolidine-1-carboxamide

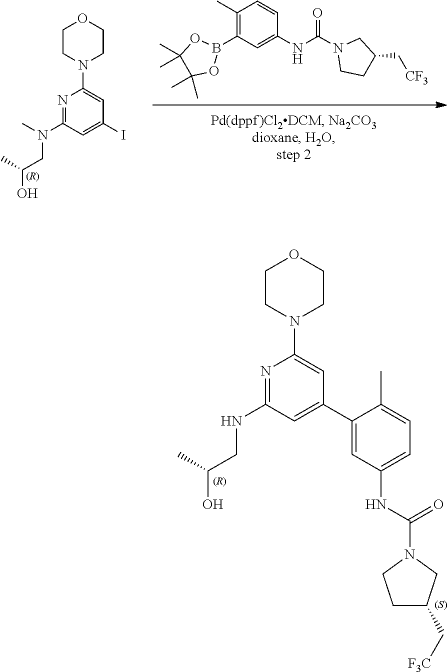

To a stirred solution of (2R)-1-[[4-iodo-6-(morpholin-4-yl)pyridin-2-yl](methyl)amino]propan-2-ol (200 mg, 0.530 mmol), (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (219 mg, 0.530 mmol) and Na₂CO₃ (169 mg, 1.591 mmol) in dioxane (1.6 mL) and H₂O (0.4 mL) was added Pd(dppf)Cl₂·CH₂Cl₂ (43 mg, 0.053 mmol) at room temperature under nitrogen atmosphere. The resulting mixture was stirred for 16 h at 60° C. under nitrogen atmosphere. The reaction was quenched by the addition of water (100 mL). The resulting mixture was extracted with EtOAc (3×50 mL). The combined organic layers were washed with saturated brine (3×50 mL), dried over anhydrous Na₂SO₄. After filtration, the filtrate was concentrated under reduced pressure. The residue was purified by silica gel column chromatography, eluted with PE/EA/EtOH (8/3/1) to afford 70 mg crude product. The residue was purified by reverse flash chromatography with the following conditions: Column: XBridge Prep C18 OBD Column, 19×150 mm Sum; Mobile Phase A:Water(10 mmol/1 NH₄HCO₃), Mobile Phase B: CH₃CN; Flow rate:20 mL/min; Gradient:40 B to 70 B in 4.3 min; 210/254 nm. This resulted in (3S)-N-[3-(2-[[(2R)-2-hydroxypropyl](methyl)amino]-6-(morpholin-4-yl)pyridin-4-yl)-4-methylphenyl]-3-(2,2,2-trifluoroethyl)pyrrolidine-1-carboxamide (144 mg, 51%) as a light green solid. MS ESI calculated for C₂₇H₃₆F₃N₅O₃[M+H]⁺, 536.28, found 536.20. ¹H NMR (400 MHz, DMSO-d₆) δ 8.11 (s, 1H), 7.44-7.40 (m, 1H), 7.33-7.32 (m, 1H), 7.11-7.08 (m, 1H), 5.84-5.79 (m, 2H), 4.66-4.65 (m, 1H), 3.92-3.86 (m, 1H), 3.69-3.63 (m, 5H), 3.55-3.44 (m, 2H), 3.40-3.37 (m, 4H), 3.29-3.26 (m, 2H), 3.02-2.98 (m, 4H), 2.46-2.39 (m, 3H), 2.15-2.06 (m, 4H), 1.71-1.61 (m, 1H), 1.06-1.04 (m, 3H). ¹⁹F NMR (376 MHz, DMSO-d₆) δ-63.35 (3F).

### Example 26: (3S)-N-[3-[2-(3,6-dihydro-2H-pyran-4-yl)-6-[[(2R)-2-hydroxypropyl]amino]pyridin-4-yl]-4-methylphenyl]-3-(2,2,2-trifluoroethyl)pyrrolidine-1-carboxamide

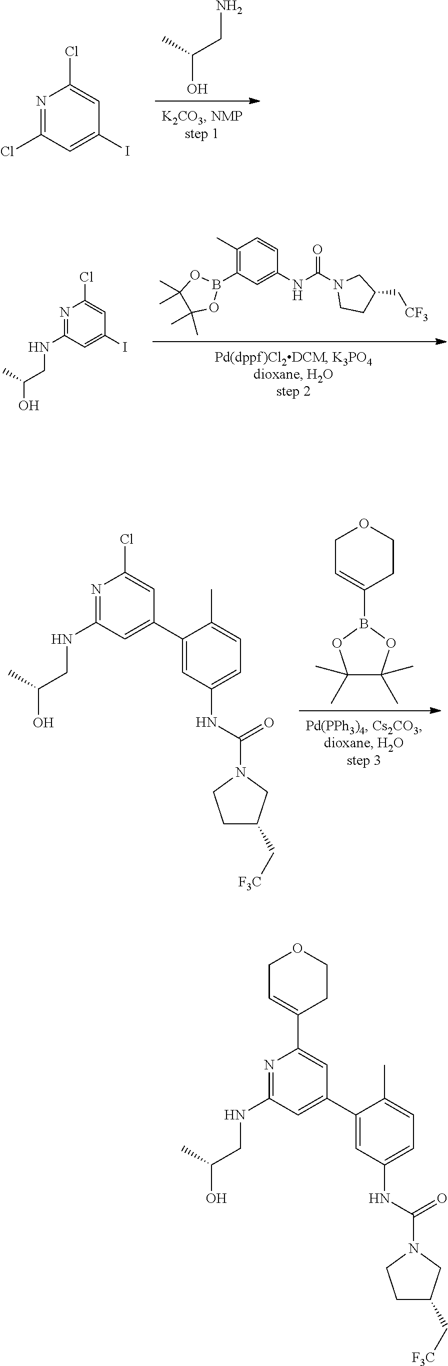

Preparation 26A: (2R)-1-[(6-chloro-4-iodopyridin-2-yl)amino]propan-2-ol

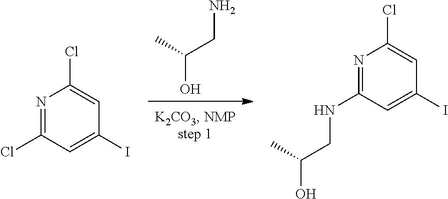

To a stirred solution of 2,6-dichloro-4-iodopyridine (1 g, 3.651 mmol) and K₂CO₃ (1 g, 7.302 mmol) in NMP (20 mL) was added (R)-1-amino-2-propanol (274 mg, 3.651 mmol) at room temperature. The part of resulting mixture was stirred for 16 h at 100° C. The reaction was quenched by the addition of water (70 mL). The resulting mixture was extracted with EtOAc (3×150 mL). The combined organic layers were washed with saturated brine (3×50 mL), dried over anhydrous Na₂SO₄. After filtration, the filtrate was concentrated under reduced pressure. The residue was purified by silica gel column chromatography, eluted with PE/EtOAc (2/1) to afford (2R)-1-[(6-chloro-4-iodopyridin-2-yl)amino]propan-2-ol (410 mg, 36%) as a yellow oil. MS ESI calculated for C₈H₁₀ClIN₂O [M+H]⁺, 312.54, found 312.95. ¹H NMR (300 MHz, chloroform-d) δ 6.96 (d, J=1.0 Hz, 1H), 6.74 (d, J=1.1 Hz, 1H), 5.31 (s, 1H), 3.46 (dd, J=13.7, 3.1 Hz, 2H), 3.19 (dd, J=13.7, 7.7 Hz, 1H), 1.26 (d, J=6.2 Hz, 3H).

Preparation 26B: (3S)-N-[3-(2-chloro-6-[[(2R)-2-hydroxypropyl]amino]pyridin-4-yl)-4-methylphenyl]-3-(2,2,2-trifluoroethyl)pyrrolidine-1-carboxamide

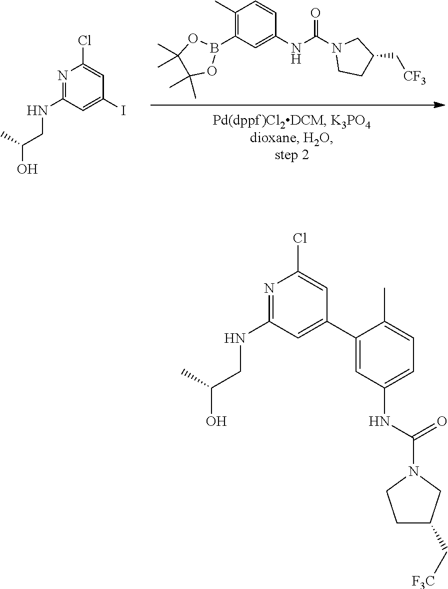 [SMILES: C[C@@H](O)CNc1cc(I)cc(Cl)n1 ; Cc1ccc(NC(=O)N2CC[C@@H](CC(F)(F)F)C2)cc1B1OC(C)(C)C(C)(C)O1 ; Cc1ccc(NC(=O)N2CC[C@@H](CC(F)(F)F)C2)cc1-c1cc(Cl)nc(NC[C@@H](C)O)c1]

To a stirred mixture of (2R)-1-[(6-chloro-4-iodopyridin-2-yl)amino]propan-2-ol (380 mg, 1.216 mmol) and (3S)-N-[4-methyl-3-(4,4,5,5-tetramethyl-1,3,2-dioxaborolan-2-yl)phenyl]-3-(2,2,2-trifluoroethyl)pyrrod-ine-1-carboxamide (501 mg, 1.216 mmol), K₃PO₄ (774 mg, 3.648 mmol) in dioxane (4 mL), H₂O (0.4 mL) was added Pd(dppf)Cl₂ CH₂Cl₂ (99 mg, 0.122 mmol) in portions at room temperature under nitrogen atmosphere. The resulting mixture was stirred for 1 h at 50° C. under nitrogen atmosphere. The resulting mixture was quenched with water (100 mL), and extracted with EtOAc (3×300 mL). The combined organic layers were washed with brine (2×70 mL), dried over anhydrous Na₂SO₄ and concentrated under redunced pressure. The residue was purified by silica gel column chromatography, eluted with 0% to 50% EtOAc in PE to afford (3S)-N-[3-(2-chloro-6-[[(2R)-2-hydroxypropyl]amino]pyridin-4-yl)-4-methylphenyl]-3-(2,2,2-trifluoroethyl)pyrrolidine-1-carboxamide (510 mg, 89%) as a yellow solid. MS ESI calculated for C₂₂H₂₆ClF₃N₄O₂ [M+H]⁺, 471.17, found 471.20. ¹H NMR (300 MHz, DMSO-d₆) δ 8.19 (s, 1H), 7.49 (s, 1H), 7.41 (s, 1H), 7.15 (s, 1H), 7.00 (s, 1H), 6.44 (d, J=1.1 Hz, 2H), 4.75 (s, 1H), 4.05-3.98 (m, 1H), 3.69-3.60 (m, 1H), 3.55-3.51 (m, 1H), 3.33-3.29 (m, 2H), 3.21-3.18 (m, 2H), 2.19 (s, 1H), 2.16-2.05 (m, 2H), 2.01 (d, J=1.1 Hz, 1H), 1.67-1.61 (m, 2H), 1.07 (s, 3H).

### Example 26: (3S)-N-[3-[2-(3,6-dihydro-2H-pyran-4-yl)-6-[[(2R)-2-hydroxypropyl]amino]pyridin-4-yl]-4-methylphenyl]-3-(2,2,2-trifluoroethyl)pyrrolidine-1-carboxamide

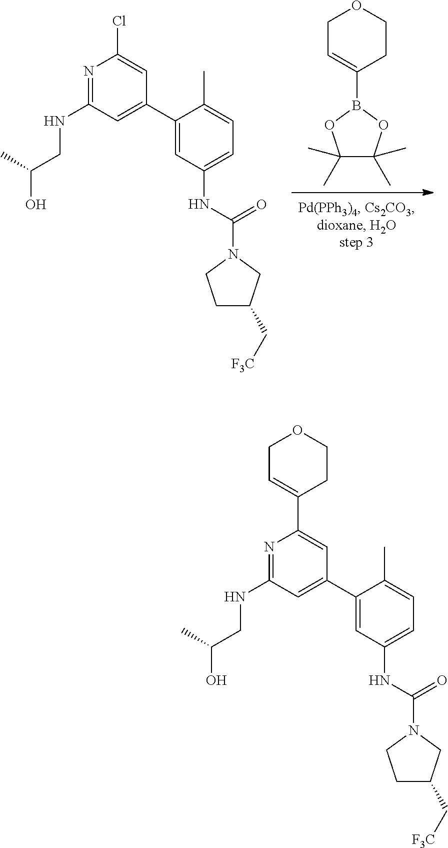

To a stirred mixture of (3S)-N-[3-(2-chloro-6-[[(2R)-2-hydroxypropyl]amino]pyridin-4-yl)-4-methylphenyl]-3-(2,2,2-trifluoroethyl)pyrrolidine-1-carboxamide (430 mg, 0.913 mmol) and 2-(3,6-dihydro-2H-pyran-4-yl)-4,4,5,5-tetramethyl-1,3,2-dioxaborolane (384 mg, 1.826 mmol), Cs₂CO₃ (893 mg, 2.739 mmol) in dioxane (5 mL), H₂O (0.5 mL) was added Pd(dppf)Cl₂ CH₂Cl₂ (75 mg, 0.091 mmol) in portions at room temperature under nitrogen atmosphere. The resulting mixture was stirred for 1 h at 100° C. under nitrogen atmosphere. The resulting mixture was quenched with water (50 mL), and extracted with EtOAc (3×100 mL). The combined organic layers were washed with brine (2×30 mL), dried over anhydrous Na₂SO₄ and concentrated under redunced pressure. The residue was purified by silica gel column chromatography, eluted with 0% to 90% EtOAc in PE to afford (3S)-N-[3-[2-(3,6-dihydro-2H-pyran-4-yl)-6-[[(2R)-2-hydroxypropyl]amino]pyridin-4-yl]-4-methylphenyl]-3-(2,2,2-trifluoroethyl)pyrrolidine-1-carboxamide (310 mg, 65%) as a yellow solid. MS ESI calculated for C₂₇H₃₃F₃N₄O₃ [M+H]⁺, 519.25, found 519.15. ¹H NMR (300 MHz, DMSO-d₆) δ 8.17 (s, 1H), 7.47-7.45 (m, 2H), 7.40 (s, 1H), 7.16-7.13 (m, 1H), 6.73 (s, 1H), 6.53 (s, 1H), 6.36 (s, 1H), 4.84-4.83 (m, 1H), 4.27 (s, 2H), 3.81 (s, 3H), 3.71-3.69 (m, 2H), 3.57-3.31 (m, 4H), 3.07-3.01 (m, 1H), 2.52-2.42 (m, 4H), 2.18-2.10 (m, 4H), 1.70-1.64 (m, 1H), 1.12-1.10 (m, 3H).

### Example 27: (3S)-N-[3-(2-[[(2R)-2-hydroxypropyl]amino]-6-(oxan-4-yl)pyridin-4-yl)-4-methylphenyl]-3-(2,2,2-trifluoroethyl)pyrrolidine-1-crboxamide

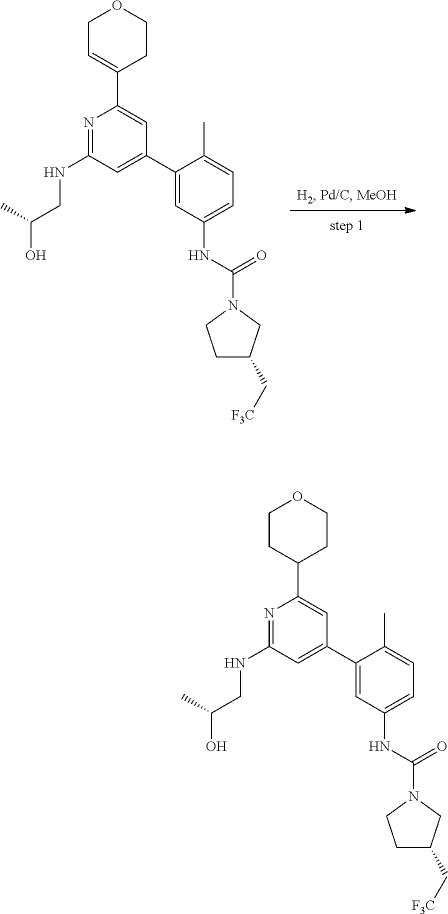 [SMILES: Cc1ccc(NC(=O)N2CC[C@@H](CC(F)(F)F)C2)cc1-c1cc(NC[C@@H](C)O)nc(C2=CCOCC2)c1 ; Cc1ccc(NC(=O)N2CC[C@@H](CC(F)(F)F)C2)cc1-c1cc(NC[C@@H](C)O)nc(C2CCOCC2)c1]

To a solution of (3S)-N-[3-[2-(3,6-dihydro-2H-pyran-4-yl)-6-[[(2R)-2-hydroxypropyl]amino]pyridin-4-yl]-4-methylphenyl]-3-(2,2,2-trifluoroethyl)pyrrolidine-1-carboxamide (150 mg, 0.289 mmol) in MeOH (2 mL), CH₃COOH (0.1 mL) was added Pd/C (92 mg, 0.868 mmol) under nitrogen atmosphere. The mixture was hydrogenated at room temperature for 2 h under hydrogen atmosphere using a hydrogen balloon, filtered through a Celite pad and concentrated under reduced pressure. The crude product (200 mg) was purified by Prep-HPLC with the following conditions (Column: XBridge Prep C18 OBD Column, 150 mm 5 um; Mobile Phase A:Water(10 mmol/L NH₄HCO₃), Mobile Phase B: CH₃CN; Flow rate:20 mL/min; Gradient:20 B to 50 B in 4.3 min; 210/254 nm) to afford (3S)-N-[3-(2-[[(2R)-2-hydroxypropyl]amino]-6-(oxan-4-yl)pyridin-4-yl)-4-methylphenyl]-3-(2,2,2-trifluoroethyl)pyrrolidine-1-carboxamide (115 mg, 76%) as a white solid. MS ESI calculated for C₂₇H₃₅F₃N₄O₃[M+H]⁺, 521.27, found 521.15. ¹H NMR (300 MHz, DMSO-d₆) δ 8.14 (s, 1H), 7.41 (s, 2H), 7.36-7.35 (m, 1H), 7.13-7.10 (m, 1H), 6.29-6.27 (m, 2H), 4.84 (s, 1H), 3.95 (s, 2H), 3.91 (s, 1H), 3.41 (s, 1H), 3.37-3.33 (m, 3H), 3.27-3.25 (m, 2H), 3.10-2.98 (m, 1H), 2.78 (s, 1H), 2.50-2.42 (m, 4H), 2.15-2.10 (m, 4H), 1.75-1.71 (m, 5H), 1.09-1.07 (m, 3H).

### Example 28: N-[3-(2-[[(2R)-2-hydroxypropyl]amino]-6-(morpholin-4-yl)pyridin-4-yl)-4-methylphenyl]-3-(trifluoromethyl)-2,5-dihydropyrrole-1-carboxamide

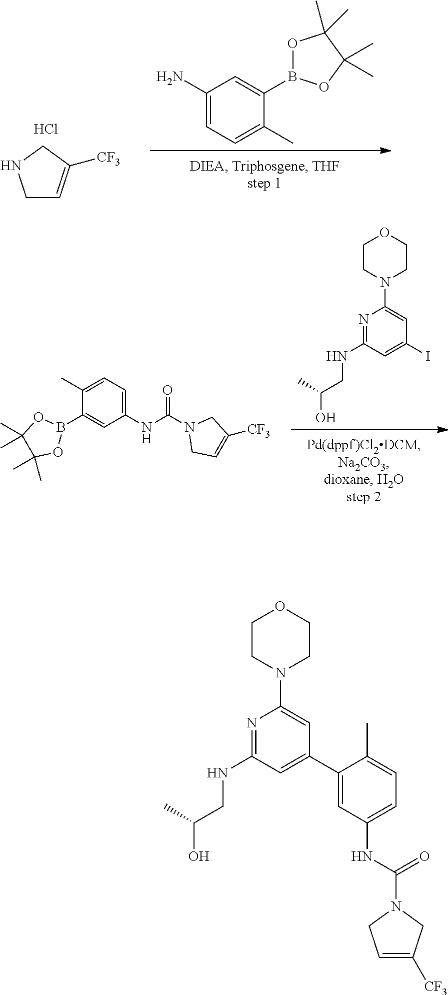 [SMILES: Cl ; Cc1ccc(N)cc1B1OC(C)(C)C(C)(C)O1 ; FC(F)(F)C1=CCNC1 ; Cc1ccc(NC(=O)N2CC=C(C(F)(F)F)C2)cc1B1OC(C)(C)C(C)(C)O1 ; C[C@@H](O)CNc1cc(I)cc(N2CCOCC2)n1 ; Cc1ccc(NC(=O)N2CC=C(C(F)(F)F)C2)cc1-c1cc(NC[C@@H](C)O)nc(N2CCOCC2)c1]

Preparation 28A: N-[4-methyl-3-(4,4,5,5-tetramethyl-1,3,2-dioxaborolan-2-yl)phenyl]-3-(trifluoromethyl)-2,5-dihydropyrrole-1-carboxamide

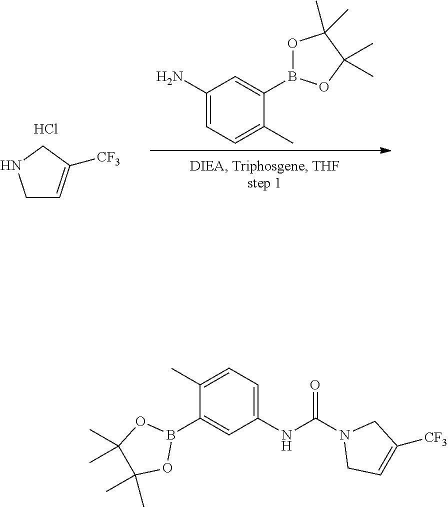 [SMILES: Cl ; Cc1ccc(N)cc1B1OC(C)(C)C(C)(C)O1 ; FC(F)(F)C1=CCNC1 ; Cc1ccc(NC(=O)N2CC=C(C(F)(F)F)C2)cc1B1OC(C)(C)C(C)(C)O1]

To a solution of 4-methyl-3-(4,4,5,5-tetramethyl-1,3,2-dioxaborolan-2-yl)aniline (435 mg, 1.867 mmol) and triphosgene (246 mg, 0.830 mmol) in THF (40 mL) was added DIEA (1.81 mL, 13.977 mmol) at room temperature under nitrogen atmosphere. The mixture was stirred for 30 min at room temperature. To this was added 3-(trifluoromethyl)-2,5-dihydro-1H-pyrrole hydrochloride (180 mg, 1.037 mmol) and the mixture was stirred for 3 h at room temperature. The resulting mixture was concentrated under reduced pressure. The residue was purified by silica gel column chromatography, eluted with PE/EtOAc (2/1) to afford N-[4-methyl-3-(4,4,5,5-tetramethyl-1,3,2-dioxaborolan-2-yl)phenyl]-3-(trifluoromethyl)-2,5-dihydropyrrole-1-carboxamide (500 mg, 61%) as a yellow oil. MS ESI calculated for C₁₉H₂₄F₃N₂O₃[M+H]⁺, 397.25, found 397.25. ¹H NMR (400 MHz, CDCl₃-d) δ 7.71 (dd, J=8.3, 2.5 Hz, 1H), 7.48 (d, J=2.6 Hz, 1H), 7.15 (d, J=8.3 Hz, 1H), 6.45-6.40 (m, 1H), 6.16 (s, 1H), 4.44 (s, 4H), 2.50 (s, 3H), 1.36 (s, 12H). ¹⁹F NMR (376 MHz, CDCl₃-d) 6-65.69 (3F).

### Example 28: N-[3-(2-[[(2R)-2-hydroxypropyl]amino]-6-(morpholin-4-yl)pyridin-4-yl)-4-methylphenyl]-3-(trifluoromethyl)-2,5-dihydropyrrole-1-carboxamide

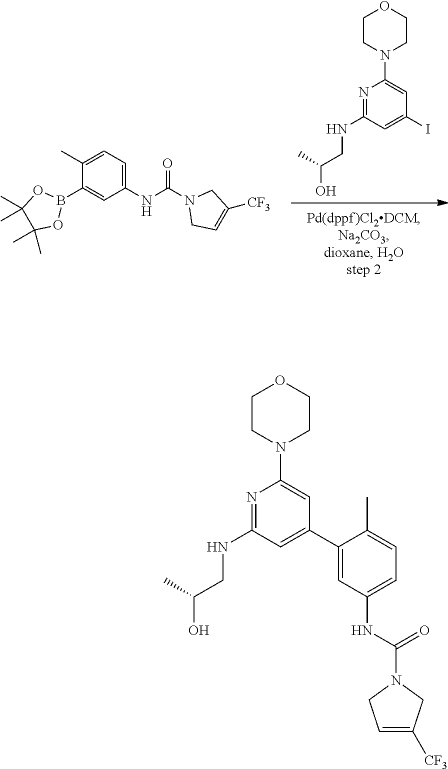 [SMILES: Cc1ccc(NC(=O)N2CC=C(C(F)(F)F)C2)cc1B1OC(C)(C)C(C)(C)O1 ; C[C@@H](O)CNc1cc(I)cc(N2CCOCC2)n1 ; Cc1ccc(NC(=O)N2CC=C(C(F)(F)F)C2)cc1-c1cc(NC[C@@H](C)O)nc(N2CCOCC2)c1]

To a stirred solution of (2R)-1-[[4-iodo-6-(morpholin-4-yl)pyridin-2-yl]amino]propan-2-ol (160 mg, 0.441 mmol) and N-[4-methyl-3-(4,4,5,5-tetramethyl-1,3,2-dioxaborolan-2-yl)phenyl]-3-(trifluoromethyl)-2,5-dihydropyrrole-1-carboxamide (174 mg, 0.441 mmol) in 1,4-dioxane (2 mL) and H₂O (0.5 mL) were added Pd(dppf)Cl₂—CH₂Cl₂ (36 mg, 0.044 mmol) and Na₂CO₃ (140 mg, 1.322 mmol). The resulting mixture was stirred for 16 h at 60° C. under nitrogen atmosphere. The mixture was allowed to cool down to room temperature. The reaction was quenched by the addition of water (10 mL) at room temperature. The resulting mixture was extracted with EtOAc (3×10 mL). The combined organic layers were washed with brine (10 mL), dried over anhydrous Na₂SO₄. After filtration, the filtrate was concentrated under reduced pressure. The residue was purified by silica gel column chromatography, eluted with PE/EtOAc (1/2) to afford N-[3-(2-[[(2R)-2-hydroxypropyl]amino]-6-(morpholin-4-yl)pyridin-4-yl)-4-methylphenyl]-3-(trifluoromethyl)-2,5-dihydropyrrole-1-carboxamide (116 mg, 44%) as a light green solid. MS ESI calculated for C₂₅H₃₀F₃N₅O₃ [M+H]⁺, 506.05, found 506.05. ¹H NMR (300 MHz, DMSO-d₆) δ 8.31 (s, 1H), 7.45 (dd, J=8.2, 2.3 Hz, 1H), 7.37 (d, J=2.3 Hz, 1H), 7.14 (d, J=8.3 Hz, 1H), 6.75 (d, J=2.6 Hz, 1H), 6.23 (s, 1H), 5.79 (d, J=6.5 Hz, 2H), 4.70 (s, 1H), 4.41 (s, 4H), 3.81 (s, 1H), 3.70 (t, J=4.8 Hz, 4H), 3.39 (t, J=4.8 Hz, 4H), 3.23-3.09 (m, 2H), 2.19 (s, 3H), 1.10 (d, J=6.2 Hz, 3H). ¹⁹F NMR (282 MHz, DMSO-d₆) δ-63.98 (3F).

### Example 29: N-[3-(2-[[(2R)-1-hydroxypropan-2-yl]amino]-6-(morpholin-4-yl)pyridin-4-yl)-4-methylphenyl]-3-(trifluoromethyl)-2,5-dihydropyrrole-1-carboxamide

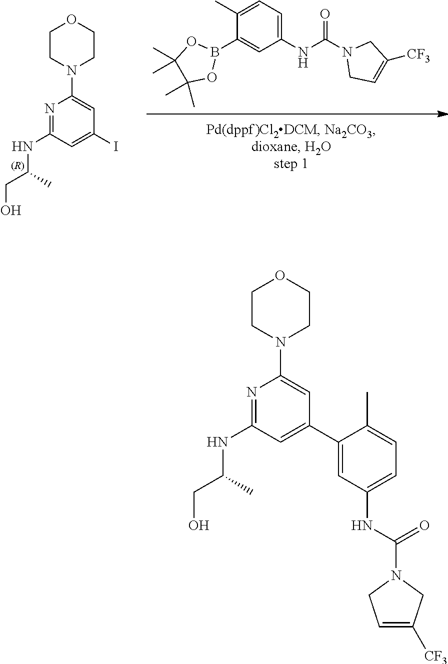

To a stirred solution of (2R)-2-[[4-iodo-6-(morpholin-4-yl)pyridin-2-yl]amino]propan-1-ol (150 mg, 0.413 mmol) and N-[4-methyl-3-(4,4,5,5-tetramethyl-1,3,2-dioxaborolan-2-yl)phenyl]-3-(trifluoromethyl)-2,5-dihydropyrrole-1-carboxamide (164 mg, 0.413 mmol) in 1,4-dioxane (2 mL) and H₂O (0.4 mL) were added Na₂CO₃ (131 mg, 1.239 mmol) and Pd(dppf)Cl₂·CH₂Cl₂ (34 mg, 0.041 mmol). The resulting mixture was stirred for 16 h at 60° C. under nitrogen atmosphere. The mixture was allowed to cool down to room temperature. The reaction was quenched by the addition of water (10 mL) at room temperature. The resulting mixture was extracted with EtOAc (3×10 mL). The combined organic layers were washed with brine (10 mL), dried over anhydrous Na₂SO₄. After filtration, the filtrate was concentrated under reduced pressure. The residue was purified by silica gel column chromatography, eluted with PE/EtOAc (1/2) to afford N-[3-(2-[[(2R)-1-hydroxypropan-2-yl]amino]-6-(morpholin-4-yl)pyridin-4-yl)-4-methylphenyl]-3-(trifluoromethyl)-2,5-dihydropyrrole-1-carboxamide (116 mg, 56%) as a light yellow solid. MS ESI calculated for C₂₅H₃₀F₃N₅O₃ [M+H]⁺, 506.10, found 506.10. ¹H NMR (400 MHz, DMSO-d₆) δ 8.29 (s, 1H), 7.45-7.40 (m, 1H), 7.36 (d, J=2.3 Hz, 1H), 7.13 (d, J=8.4 Hz, 1H), 6.76-672 (m, 1H), 5.99 (s, 1H), 5.76 (s, 2H), 4.65 (s, 1H), 4.39 (s, 4H), 3.93-3.85 (m, 1H), 3.71-3.64 (m, 4H), 3.49 (dd, J=10.3, 4.9 Hz, 1H), 3.37 (t, J=4.8 Hz, 4H), 3.34-3.33 (m, 1H), 2.17 (s, 3H), 1.13 (d, J=6.5 Hz, 3H). ¹⁹F NMR (376 MHz, DMSO-d₆) δ-63.99 (3F).

### Example 30: (3S)-N-[3-(2-[[(2S)-1-hydroxypropan-2-yl]amino]-6-(morpholin-4-yl)pyridin-4-yl)-4-methylphenyl]-3-(2,2,2-trifluoroethyl)pyrrolidine-1-carboxamide

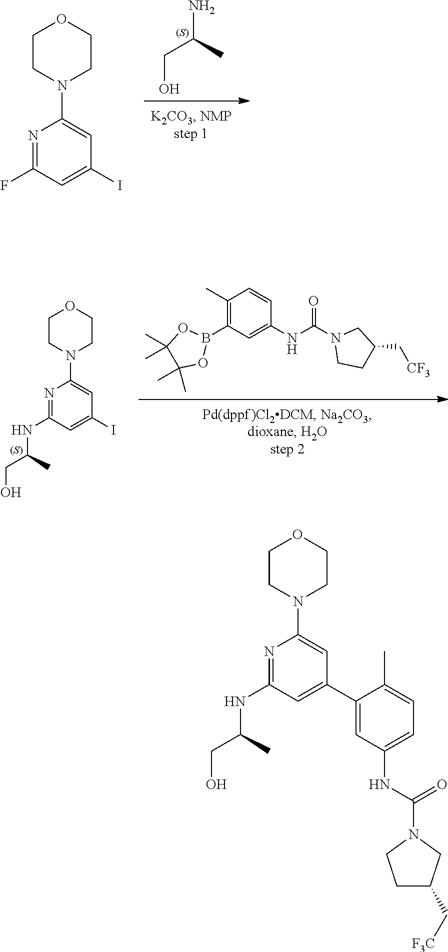

Preparation 30A: (2S)-2-[[4-iodo-6-(morpholin-4-yl)pyridin-2-yl]amino]propan-1-ol

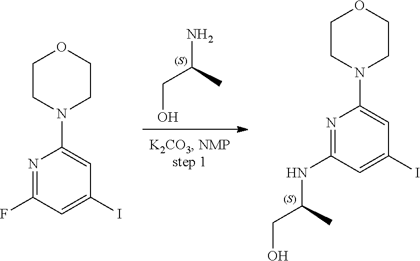

To a stirred solution of 4-(6-fluoro-4-iodopyridin-2-yl)morpholine (300 mg, 0.974 mmol) and K₂CO₃ (269 mg, 1.947 mmol) in NMP (3 mL) was added (2S)-2-aminopropan-1-ol (110 mg, 1.461 mmol) at room temperature under nitrogen atmosphere. The resulting mixture was stirred for 16 h at 150° C. under nitrogen atmosphere. The reaction was quenched by the addition of water (100 mL). The resulting mixture was extracted with EtOAc (3×50 mL). The combined organic layers were washed with brine (3×50 mL), dried over anhydrous Na₂SO₄. After filtration, the filtrate was concentrated under reduced pressure. The residue was purified by silica gel column chromatography, eluted with PE/EtOAc (1/1) to afford (2S)-2-[[4-iodo-6-(morpholin-4-yl)pyridin-2-yl]amino]propan-1-ol (198 mg, 55%) as an off-white solid. MS ESI calculated for C₁₂H₁₈IN₃O₂[M+H]⁺, 364.04, found 364.10. ¹H NMR (400 MHz, CDCL₃) δ 6.29-6.25 (m, 2H), 4.32-4.28 (m, 1H), 4.02 (s, 1H), 3.81-3.72 (m, 5H), 3.58-3.54 (m, 1H), 3.45-3.43 (m, 4H), 1.23-1.22 (m, 3H).

### Example 30: (3S)-N-[3-(2-[[(2S)-1-hydroxypropan-2-yl]amino]-6-(morpholin-4-yl)pyridin-4-yl)-4-methylphenyl]-3-(2,2,2-trifluoroethyl)pyrrolidine-1-carboxamide

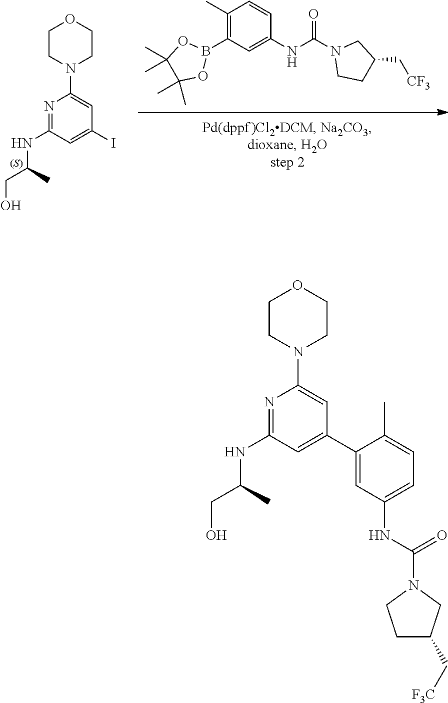

A mixture of (2S)-2-[[4-iodo-6-(morpholin-4-yl)pyridin-2-yl]amino]propan-1-ol (200 mg, 0.551 mmol), (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (227 mg, 0.551 mmol), Na₂CO₃ (175 mg, 1.652 mmol), 1,4-dioxane (1.6 mL), H₂O (0.4 mL) and Pd(dppf)Cl₂·CH₂Cl₂ (45 mg, 0.055 mmol) was stirred for 16 h at 60° C. under nitrogen atmosphere. The reaction was quenched by the addition of water (100 mL). The resulting mixture was extracted with EtOAc (3×50 mL). The combined organic layers were washed with saturated brine (3×50 mL), dried over anhydrous Na₂SO₄. After filtration, the filtrate was concentrated under reduced pressure. The residue was purified by silica gel column chromatography, eluted with PE/EtOAc/EtOH=(8/3/1) followed by reverse flash chromatography with the following conditions: Column: SunFire Prep C18 OBD Column, 19×150 mm 5 μm 10 nm; Mobile Phase A: water (0.05% TFA), Mobile Phase B: CH₃CN; Flow rate:20 mL/min; Gradient:25 B to 65 B in 5.3 min; 254/210 nm. This resulted in (3S)-N-[3-(2-[[(2S)-1-hydroxypropan-2-yl]amino]-6-(morpholin-4-yl)pyridin-4-yl)-4-methylphenyl]-3-(2,2,2-trifluoroethyl)pyrrolidine-1-carboxamide (111 mg, 37%) as an off-white solid. MS ESI calculated for C₂₆H₃₄F₃N₅O₃[M+H]⁺, 522.26, found 522.30. ¹H NMR (400 MHz, DMSO-d₆) δ 8.11 (s, 1H), 7.42-7.40 (m, 1H), 7.34-7.33 (m, 1H), 7.10-7.08 (m, 1H), 5.98-5.96 (m, 1H), 5.75 (m, 2H), 4.65 (s, 1H), 3.90-3.87 (m, 1H), 3.69-3.65 (m, 5H), 3.55-3.48 (m, 2H), 3.38-3.34 (m, 5H), 3.04-2.99 (m, 1H), 2.48-2.41 (m, 3H), 2.16 (s, 3H), 2.10-2.07 (m, 1H), 1.70-1.60 (m, 1H), 1.13-1.12 (m, 3H). ¹⁹F NMR (376 MHz, DMSO-d₆) δ-63.37 (3F).

### Example 31: (3S)-N-[3-(2-[[(2R)-2-hydroxypropyl]methyl)amino]-6-(morpholin-4-yl)pyridin-4-yl)-4-methylphenyl]-3-(2,2,2-trifluoroethyl)pyrrolidine-1-carboxamide

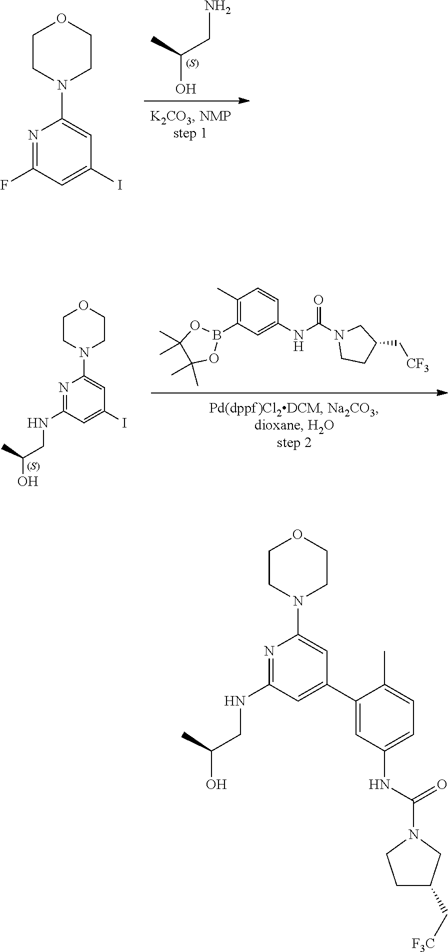

Preparation 31A: (2R)-1-[[4-iodo-6-(morpholin-4-yl)pyridin-2-yl](methyl)amino]propan-2-ol

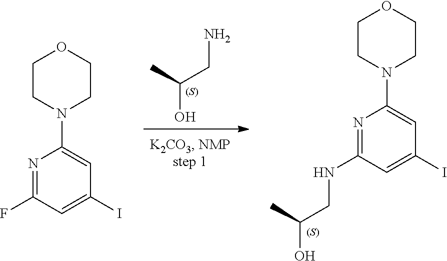

A mixture of 4-(6-fluoro-4-iodopyridin-2-yl)morpholine (300 mg, 0.974 mmol), K₂CO₃ (269 mg, 1.947 mmol), NMP (3 mL) and (2S)-1-aminopropan-2-ol (110 mg, 1.461 mmol) was stirred for 16 h at 150° C. under nitrogen atmosphere. The reaction was quenched by the addition of water (100 mL). The resulting mixture was extracted with EtOAc (3×50 mL). The combined organic layers were washed with saturated brine (3×50 mL), dried over anhydrous Na₂SO₄. After filtration, the filtrate was concentrated under reduced pressure. The residue was purified by silica gel column chromatography, eluted with PE/EtOAc (1/1) to afford (2S)-1-[[4-iodo-6-(morpholin-4-yl)pyridin-2-yl]amino]propan-2-ol (222 mg, 63%) as an off-white solid. MS ESI calculated for C₁₂H₁₈IN₃O₂[M+H]⁺, 364.04, found 364.10. ¹H NMR (400 MHz, chloroform-d) δ 6.29-6.25 (m, 2H), 4.65 (s, 1H), 4.05-3.97 (m, 1H), 3.81-3.78 (m, 4H), 3.47-3.41 (m, 5H), 3.25-3.18 (m, 1H), 1.25-1.23 (m, 3H).

### Example 31: (3S)-N-[3-(2-[[(2R)-2-hydroxypropyl](methyl)amino]-6-(morpholin-4-yl)pyridin-4-yl)-4-methylphenyl]-3-(2,2,2-trifluoroethyl)pyrrolidine-1-carboxamide

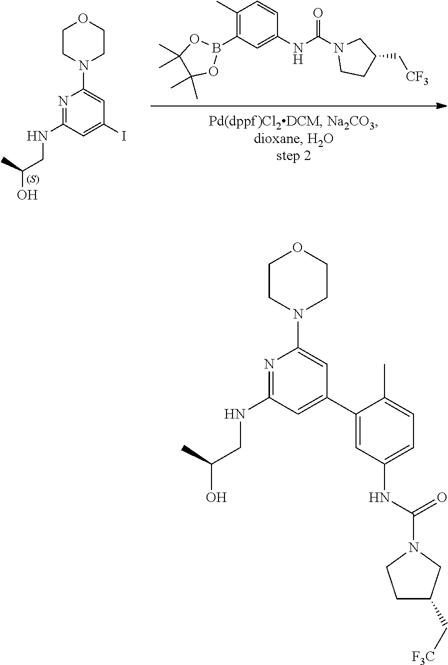

A mixture of (2S)-1-[[4-iodo-6-(morpholin-4-yl)pyridin-2-yl]amino]propan-2-ol (200 mg, 0.551 mmol), (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (227 mg, 0.551 mmol), Na₂CO₃ (175 mg, 1.652 mmol), dioxane:H₂O=4:1 (2 mL) and Pd(dppf)Cl₂·CH₂Cl₂ (45 mg, 0.055 mmol) was stirred for 16 h at 60° C. under nitrogen atmosphere. The reaction was quenched by the addition of water (100 mL).

The resulting mixture was extracted with EtOAc (3×50 mL). The combined organic layers were washed with saturated brine (3×50 mL), dried over anhydrous Na₂SO₄. After filtration, the filtrate was concentrated under reduced pressure. The residue was purified by silica gel column chromatography, eluted with PE/EA/EtOH=(8/3/1) followed by reverse flash chromatography with the following conditions: Column: SunFire Prep C18 OBD Column, 19×150 mm Sum 10 nm; Mobile Phase A: water (0.1% FA), Mobile Phase B: CH₃CN; Flow rate:20 mL/min; Gradient:25 B to 65 B in 5.3 min; 254/210 nm. This resulted in (3S)-N-[3-(2-[[(2S)-2-hydroxypropyl]amino]-6-(morpholin-4-yl)pyridin-4-yl)-4-methylphenyl]-3-(2,2,2-trifluoroethyl)pyrrolidine-1-carboxamide (145 mg, 49%) as an off-white solid. MS ESI calculated for C₂₆H₃₄F₃N₅O₃[M+H]⁺, 522.26, found 522.25. ¹H NMR (400 MHz, DMSO-d₆) δ 8.11 (s, 1H), 7.43-7.40 (m, 1H), 7.34-7.33 (m, 1H), 7.10-7.08 (m, 1H), 6.22-6.19 (m, 1H), 5.78-5.76 (m, 2H), 4.70 (s, 1H), 3.82-3.77 (m, 1H), 3.69-3.65 (m, 5H), 3.55-3.50 (m, 1H), 3.38-3.36 (m, 4H), 3.23-3.11 (m, 2H), 3.04-2.99 (m, 1H), 2.48-2.36 (m, 3H), 2.16 (s, 3H), 2.11-2.07 (m, 1H), 1.70-1.60 (m, 1H), 1.09-1.07 (m, 3H). ¹⁹F NMR (376 MHz, DMSO-d₆) δ-63.37 (3F).

### Example 32: (3S)-N-[3-[2-(3,6-dihydro-2H-pyran-4-yl)-6-[[(2R)-1-hydroxypropan-2-yl]amino]pyridin-4-yl]-4-methylphenyl]-3-(2,2,2-trifluoroethyl)pyrrolidine-1-carboxamide

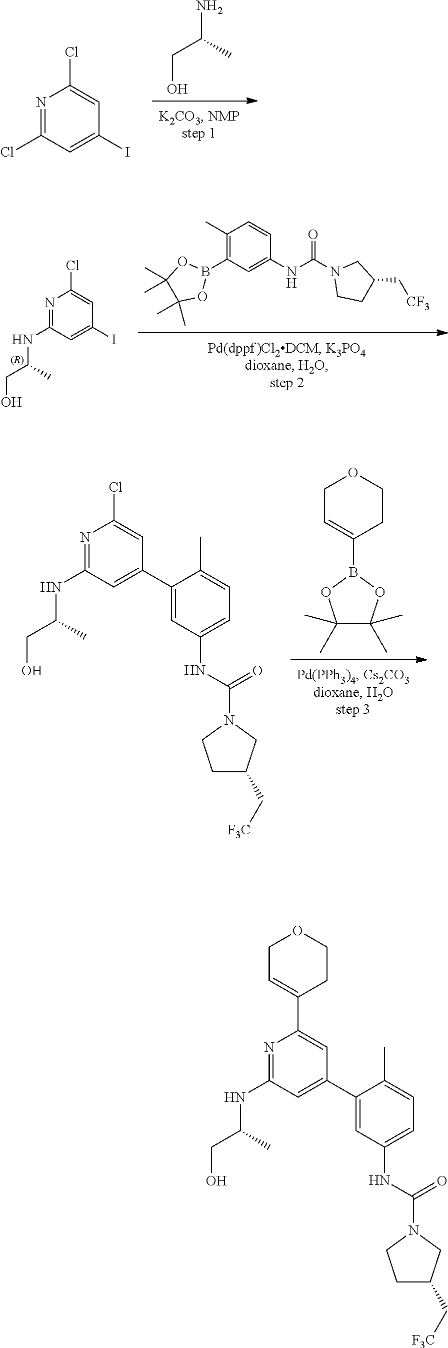

Preparation 32A: (2R)-2-[(6-chloro-4-iodopyridin-2-yl)amino]propan-1-ol

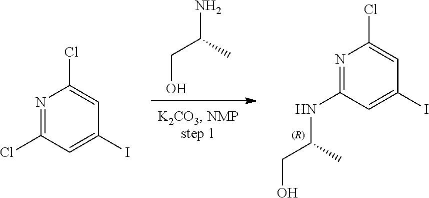

To a stirred solution of 2,6-dichloro-4-iodopyridine (1 g, 3.651 mmol) and K₂CO₃ (1 g, 7.302 mmol) in NMP (20 mL) was added (R)-(−)-2-amino-1-propanol (274 mg, 3.651 mmol) at room temperature. The part of resulting mixture was stirred for 16 h at 100° C. The reaction was quenched by the addition of water (70 mL). The resulting mixture was extracted with EtOAc (3×150 mL). The combined organic layers were washed with saturated brine (3×50 mL), dried over anhydrous Na₂SO₄. After filtration, the filtrate was concentrated under reduced pressure. The residue was purified by silica gel column chromatography, eluted with PE/EtOAc (2/1) to afford (2R)-2-[(6-chloro-4-iodopyridin-2-yl)amino]propan-1-ol (240 mg, 21%) as a yellow oil. MS ESI calculated for C₈H₁₀ClIN₂O [M+H]⁺, 312.95, found 312.95. ¹H NMR (300 MHz, chloroform-d) δ 6.97 (d, J=1.0 Hz, 1H), 6.77 (d, J=1.1 Hz, 1H), 4.90 (s, 1H), 3.76 (dd, J=13.7, 3.1 Hz, 2H), 3.61 (dd, J=13.7, 7.7 Hz, 1H), 1.28 (d, J=6.2 Hz, 3H).

Preparation 32B: (3S)-N-[3-(2-chloro-6-[[(2R)-1-hydroxypropan-2-yl]amino]pyridin-4-yl)-4-methylphenyl]-3-(2,2,2-trifluoroethyl)pyrrolidine-1-carboxamide

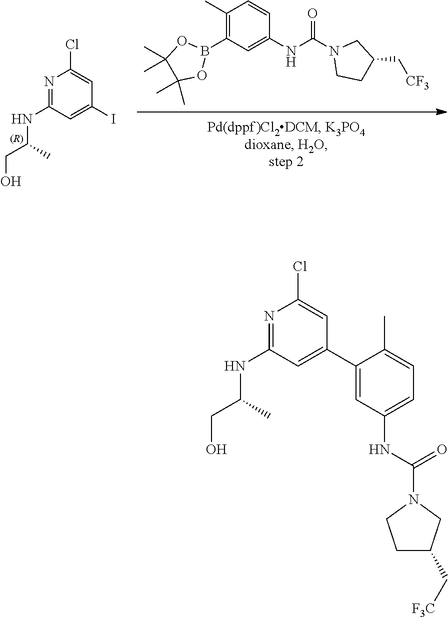

To a stirred mixture of (2R)-2-[(6-chloro-4-iodopyridin-2-yl)amino]propan-1-ol (230 mg, 0.736 mmol) and (3S)-N-[4-methyl-3-(4,4,5,5-tetramethyl-1,3,2-dioxaborolan-2-yl)phenyl]-3-(2,2,2-trifluoroethyl)pyrrol-idine-1-carboxamide (303 mg, 0.736 mmol), K₃PO₄ (469 mg, 2.208 mmol) in dioxane (3 mL), H₂O (0.3 mL) was added Pd(dppf)Cl₂ CH₂Cl₂ (60 mg, 0.074 mmol) in portions at room temperature under nitrogen atmosphere. The resulting mixture was stirred for 1 h at 50° C. under nitrogen atmosphere. The resulting mixture was quenched with water (50 mL), and extracted with EtOAc (3×100 mL). The combined organic layers were washed with brine (2×30 mL), dried over anhydrous Na₂SO₄ and concentrated under reduced pressure. The residue was purified by silica gel column chromatography, eluted with 0% to 90% EtOAc in PE to afford (3S)-N-[3-(2-chloro-6-[[(2R)-1-hydroxypropan-2-yl]amino]pyridin-4-yl)-4-methylphenyl]-3-(2,2,2-trifluoroethyl)pyrrolidine-1-carboxamide (310 mg, 89%) as a yellow solid. MS ESI calculated for C₂₂H₂₆ClF₃N₄O₂ [M+H]⁺, 471.17, found 471.05. ¹H NMR (300 MHz, DMSO-d₆) δ 8.17 (s, 1H), 7.48-7.45 (m, 1H), 7.40-7.34 (m, 1H), 7.15-7.12 (m, 1H), 6.87-6.81 (m, 1H), 6.41 (s, 1H), 7.41 (s, 1H), 6.36 (s, 1H), 4.04-4.02 (m, 1H), 3.90 (s, 1H), 3.67 (s, 1H), 3.52 (s, 2H), 3.36-3.25 (m, 2H), 3.03 (s, 1H), 2.51-2.34 (m, 3H), 2.17 (s, 3H), 2.13-2.05 (m, 1H), 1.73-1.59 (m, 1H), 1.26-1.10 (m, 4H).

### Example 32: (3S)-N-[3-[2-(3,6-dihydro-2H-pyran-4-yl)-6-[[(2R)-1-hydroxypropan-2-yl]amino]pyridin-4-yl]-4-methylphenyl]-3-(2,2,2-trifluoroethyl)pyrrolidine-1-carboxamide

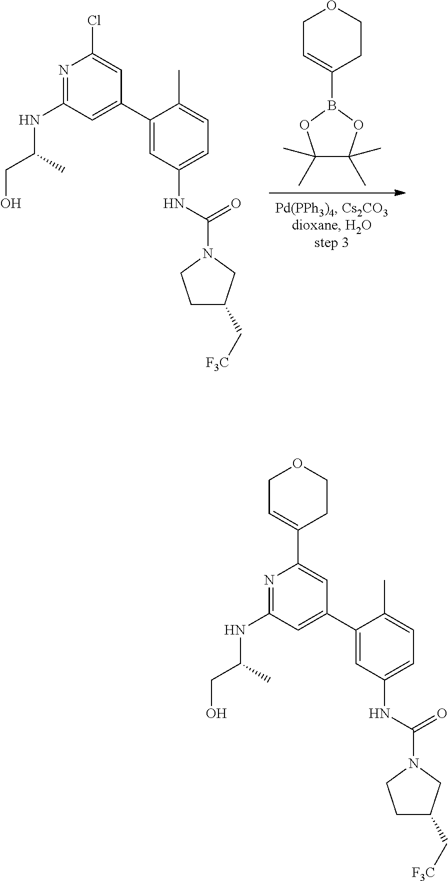

To a stirred mixture of (3S)-N-[3-(2-chloro-6-[[(2R)-1-hydroxypropan-2-yl]amino]pyridin-4-yl)-4-methylphenyl]-3-(2,2,2-trifluoroethyl)pyrrolidine-1-carboxamide (310 mg, 0.658 mmol) and 2-(3,6-dihydro-2H-pyran-4-yl)-4,4,5,5-tetramethyl-1,3,2-dioxaborolane (277 mg, 1.317 mmol), Cs₂CO₃ (643 mg, 1.975 mmol) in dioxane (4 mL), H₂O (0.4 mL) was added Pd(dppf)Cl₂ CH₂Cl₂ (54 mg, 0.066 mmol) in portions at room temperature under nitrogen atmosphere. The resulting mixture was stirred for 12 h at 100° C. under nitrogen atmosphere. The resulting mixture was quenched with water (50 mL), and extracted with EtOAc (3×100 mL). The combined organic layers were washed with brine (2×30 mL), dried over anhydrous Na₂SO₄ and concentrated under redunced pressure. The residue was purified by silica gel column chromatography, eluted with 0% to 90% EtOAc in PE to afford (3S)-N-[3-[2-(3,6-dihydro-2H-pyran-4-yl)-6-[[(2R)-1-hydroxypropan-2-yl]amino]pyridin-4-yl]-4-methylphenyl]-3-(2,2,2-trifluoroethyl)pyrrolidine-1-carboxamide (130 mg, 38%) as a yellow solid. MS ESI calculated for C₂₇H₃₃F₃N₄O₃ [M+H]⁺, 519.25, found 519.15. ¹H NMR (300 MHz, DMSO-d₆) δ 8.15 (s, 1H), 7.45-7.39 (m, 2H), 7.14-7.12 (m, 1H), 6.71 (s, 1H), 6.50 (s, 1H), 6.31 (s, 2H), 4.85-4.83 (m, 1H), 4.25-4.24 (m, 2H), 4.07 (s, 1H), 3.81 (s, 2H), 3.67-3.66 (m, 1H), 3.52-3.50 (m, 2H), 3.33-3.29 (m, 2H), 3.02 (s, 1H), 2.51-2.43 (m, 5H), 2.41-2.17 (m, 4H), 1.75-1.70 (m, 1H), 1.16-1.14 (m, 3H).

### Example 33: (3S)-N-[3-(2-[[(2R)-1-hydroxypropan-2-yl]amino]-6-(oxan-4-yl)pyridin-4-yl)-4-methylphenyl]-3-(2,2,2-trifluoroethyl)pyrrolidine-1-carboxamide

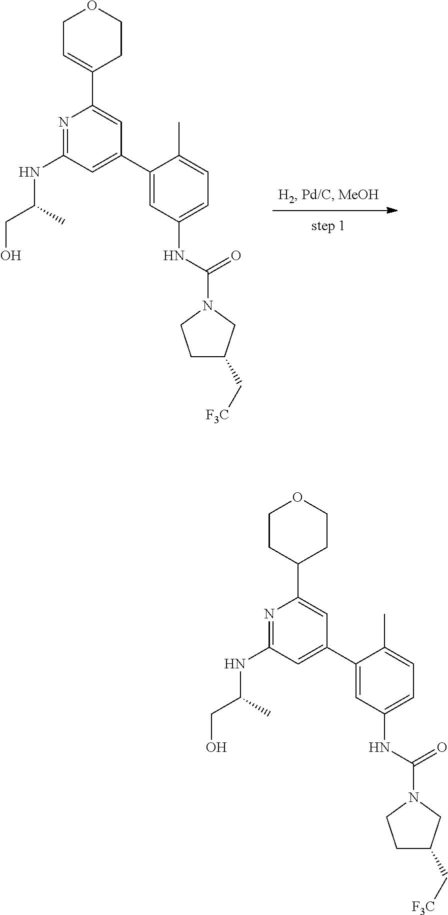 [SMILES: Cc1ccc(NC(=O)N2CC[C@@H](CC(F)(F)F)C2)cc1-c1cc(N[C@H](C)CO)nc(C2=CCOCC2)c1 ; Cc1ccc(NC(=O)N2CC[C@@H](CC(F)(F)F)C2)cc1-c1cc(N[C@H](C)CO)nc(C2CCOCC2)c1]

To a solution of (3S)-N-[3-[2-(3,6-dihydro-2H-pyran-4-yl)-6-[[(2R)-1-hydroxypropan-2-yl]amino]pyridin-4-yl]-4-methylphenyl]-3-(2,2,2-trifluoroethyl)pyrrolidine-1-carboxamide (130 mg, 0.251 mmol) in MeOH (2 mL), CH₃COOH (0.1 mL) was added Pd/C (80 mg, 0.752 mmol) under nitrogen atmosphere. The mixture was hydrogenated at room temperature for 2 h under hydrogen atmosphere using a hydrogen balloon. The reaction mixture was filtered through a celite pad and concentrated under reduced pressure. The crude product (200 mg) was purified by Prep-HPLC with the following conditions (Column: XBridge Prep C18 OBD Column, 150 mm Sum; Mobile Phase A: water (10 mmoL/L NH₄HCO₃), Mobile Phase B: CH₃CN; Flow rate:20 mL/min; Gradient:20 B to 50 B in 4.3 min; 210/254 nm) to afford (3S)-N-[3-(2-[[(2R)-1-hydroxypropan-2-yl]amino]-6-(oxan-4-yl)pyridin-4-yl)-4-methylphenyl]-3-(2,2,2-trifluoroethyl)pyrrolidine-1-carboxamide (63 mg, 48%) as a white solid. MS ESI calculated for C₂₇H₃₅F₃N₄O₃[M+H]⁺, 521.27, found 521.15. ¹H NMR (300 MHz, DMSO-d₆) δ 8.13 (s, 1H), 7.43-7.41 (m, 2H), 7.36 (s, 1H), 6.26-6.19 (m, 3H), 4.77 (s, 1H), 3.95-3.91 (m, 3H), 3.63 (s, 1H), 3.55-3.25 (m, 5H), 3.04-2.98 (m, 1H), 2.76 (s, 1H), 2.50-2.39 (m, 4H), 2.27-2.06 (m, 4H), 1.78-1.61 (m, 5H), 1.23-1.13 (m, 3H).

### Example 34: (S)-N-(3-(2-(((1S,3R)-3-hydroxy-3-methylcyclobutyl)amino)-6-morpholinopyridin-4-yl)-4-methylphenyl)-3-(2,2,2-trifluoroethyl)pyrrolidine-1-carboxamide

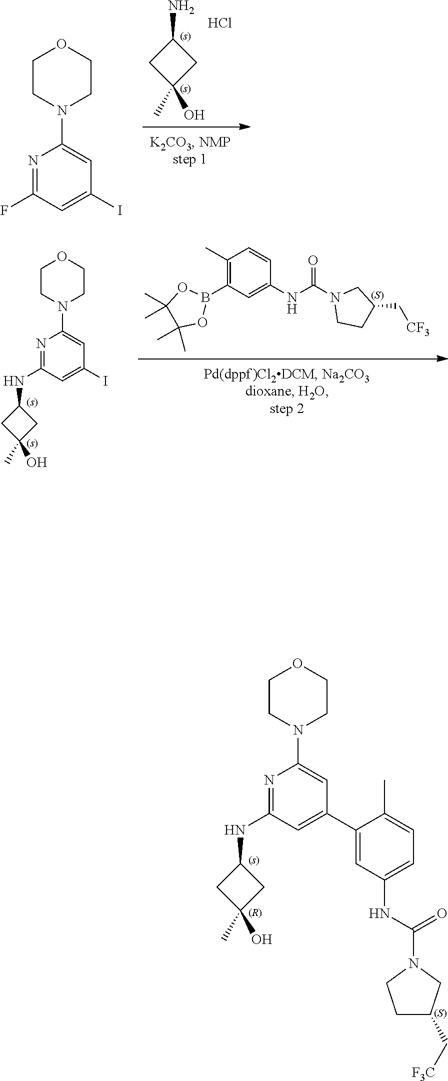

Preparation 34A: (1S,3S)-3-((4-iodo-6-morpholinopyridin-2-yl)amino)-1-methylcyclobutan-1-ol

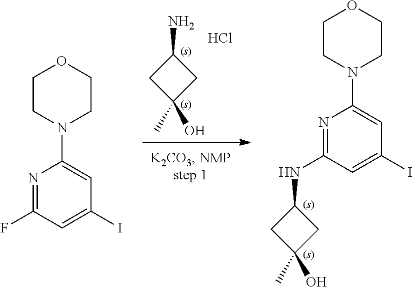

A mixture of 4-(6-fluoro-4-iodopyridin-2-yl)morpholine (300 mg, 0.974 mmol) and (1S,3S)-3-amino-1-methylcyclobutan-1-ol hydrochloride (134 mg, 0.974 mmol), K₂CO₃ (269 mg, 1.947 mmol) in NMP (3 mL) was stirred for 16 h at 150° C. under nitrogen atmosphere. The reaction was quenched with Water (2×8 mL) at room temperature. The resulting mixture was extracted with EtOAc (2×8 mL). The combined organic layers were washed with brine (10 mL), dried over anhydrous Na₂SO₄. After filtration, the filtrate was concentrated under reduced pressure. The residue was purified by silica gel column chromatography, eluted with PE/EtOAc/EtOH (4/3/1) to afford (1S,3S)-3-((4-iodo-6-morpholinopyridin-2-yl)amino)-1-methylcyclobutan-1-ol (270 mg, 64%) as an off-white solid. MS ESI calculated for C₁₄H₂AlN₃O₂[M+H]⁺, 390.06, found 390.05. ¹H NMR (300 MHz, CDCL₃) δ 6.32 (s, 1H), 6.11 (s, 1H), 4.48 (m, 1H), 3.81-3.78 (m, 4H), 3.73-3.66 (m, 1H), 3.46-3.42 (m, 4H), 2.65-2.58 (m, 1H), 2.01-1.95 (m, 2H), 1.45 (s, 3H), 1.31-1.26 (m, 1H), 0.91-0.86 (m, 1H).

### Example 34: (S)-N-(3-(2-(((1S,3R)-3-hydroxy-3-methylcyclobutyl)amino)-6-morpholinopyridin-4-yl)-4-methylphenyl)-3-(2,2,2-trifluoroethyl)pyrrolidine-1-carboxamide

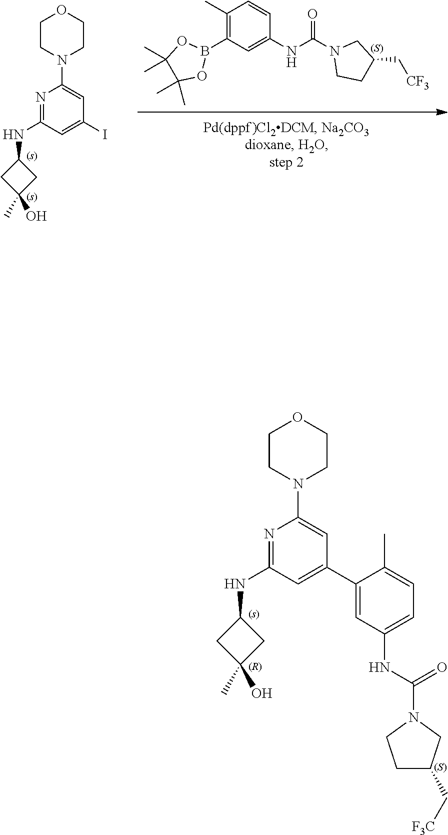

A mixture of (1S,3S)-3-((4-iodo-6-morpholinopyridin-2-yl)amino)-1-methylcyclobutan-1-ol (142 mg, 0.364 mmol), (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (150 mg, 0.364 mmol), Pd(dppf)Cl₂·CH₂Cl₂ (30 mg, 0.036 mmol) and Na₂CO₃ (116 mg, 1.092 mmol) in dioxane (1.2 mL) and H₂O (0.3 mL) was stirred for 16 h at 60° C. under nitrogen atmosphere. The reaction was quenched by the addition of water (3 mL) at room temperature. The resulting mixture was extracted with EtOAc (2×10 mL). The combined organic layers were washed with brine (5 mL), dried over anhydrous Na₂SO₄. After filtration, the filtrate was concentrated under reduced pressure. The residue was purified by silica gel column chromatography, eluted with PE/EtOAc/EtOH (4/3/1), the crude product was purified by reverse flash chromatography with the following conditions: Column: SunFire Prep C18 OBD Column, 19×150 mm Sum 10 nm; Mobile Phase A: water (0.1% FA), Mobile Phase B: CH₃CN; Flow rate:20 mL/min; Gradient:35 B to 70 B in 4.3 min; 210/254 nm. This resulted in (S)-N-(3-(2-(((1S,3R)-3-hydroxy-3-methylcyclobutyl)amino)-6-morpholinopyridin-4-yl)-4-methylphenyl)-3-(2,2,2-trifluoroethyl)pyrrolidine-1-carboxamide (117 mg, 58%) as an off-white solid. MS ESI calculated for C₂₈H₃₆F₃N₅O₃[M+H]⁺, 548.28, found 548.20. ¹H NMR (300 MHz, DMSO-d₆) δ 8.13-8.11 (m, 1H), 7.42-7.40 (m, 1H), 7.33 (s, 1H), 7.10-7.07 (m, 1H), 6.44-6.32 (m, 1H), 5.77-5.69 (m, 2H), 4.90-4.87 (m, 1H), 3.89-3.60 (m, 6H), 3.55-3.49 (m, 1H), 3.37-3.29 (m, 5H), 3.07-2.99 (m, 1H), 2.51-2.34 (m, 5H), 2.20-1.94 (m, 4H), 1.90-1.81 (m, 2H), 1.80-1.50 (m, 1H), 1.45-1.05 (m, 3H). ¹⁹F NMR (282 MHz, DMSO-d₆) δ-63.35(3F).

### Example 35: (S)-N-(3-(2-(((1R,3S)-3-hydroxy-3-methylcyclobutyl)amino)-6-morpholinopyridin-4-yl)-4-methylphenyl)-3-(2,2,2-trifluoroethyl)pyrrolidine-1-carboxamide

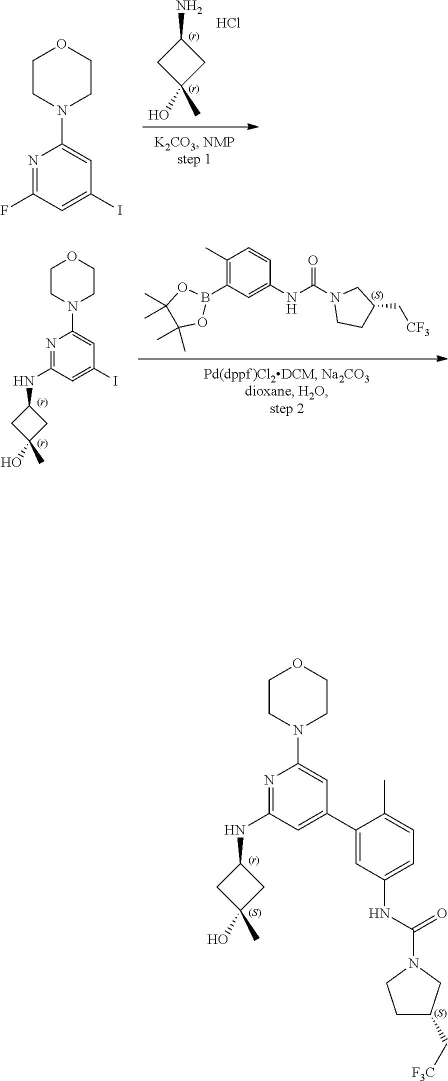

Preparation 35A: (1R,3R)-3-((4-iodo-6-morpholinopyridin-2-yl)amino)-1-methylcyclobutan-1-ol

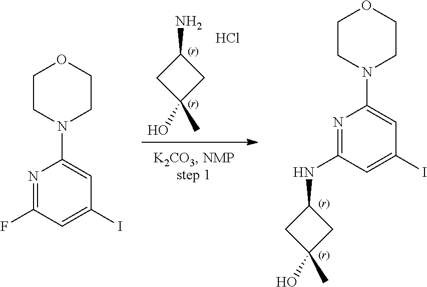

A mixture of 4-(6-fluoro-4-iodopyridin-2-yl)morpholine (300 mg, 0.974 mmol) and (trans)-3-amino-1-methylcyclobutan-1-ol hydrochloride (201 mg, 1.461 mmol), K₂CO₃ (269 mg, 1.947 mmol) in NMP (3 mL) was stirred for 16 h at 150° C. under nitrogen atmosphere. The reaction was quenched by the addition of Water (30 mL) at room temperature. The resulting mixture was extracted with EtOAc (2×20 mL). The combined organic layers were washed with brine (10 mL), dried over anhydrous Na₂SO₄. After filtration, the filtrate was concentrated under reduced pressure. The residue was purified by silica gel column chromatography, eluted with PE/EtOAc/EtOH=(4/3/1) to afford (1R,3R)-3-((4-iodo-6-morpholinopyridin-2-yl)amino)-1-methylcyclobutan-1-ol (370 mg, 88%) as an off-white solid. MS ESI calculated for C₁₄H₂AlN₃O₂ [M+H]⁺, 390.06, found 390.05. ¹H NMR (300 MHz, chloroform-d) δ 6.33-6.32 (m, 1H), 6.11-6.10 (s, 1H), 4.47-4.45 (m, 1H), 4.23-4.11 (m, 1H), 3.80-3.77 (m, 4H), 3.45-3.42 (m, 4H), 2.60-2.53 (m, 1H), 2.00-1.93 (m, 2H), 1.46 (s, 3H), 1.31-1.26 (m, 1H), 0.91-0.88 (m, 1H).

### Example 35: (S)-N-(3-(2-(((1R,3S)-3-hydroxy-3-methylcyclobutyl)amino)-6-morpholinopyridin-4-yl)-4-methylphenyl)-3-(2,2,2-trifluoroethyl)pyrrolidine-1-carboxamide

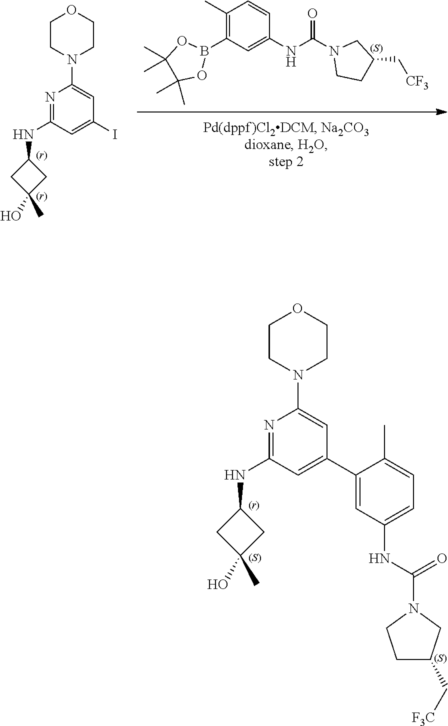

A mixture of (1R,3R)-3-((4-iodo-6-morpholinopyridin-2-yl)amino)-1-methylcyclobutan-1-ol (142 mg, 0.364 mmol), (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (150 mg, 0.364 mmol), Pd(dppf)Cl₂·CH₂Cl₂ (30 mg, 0.036 mmol) and Na₂CO₃ (116 mg, 1.092 mmol) in dioxane (1.2 mL) and H₂O (0.3 mL) was stirred for 16 h at 60° C. under hydrogen atmosphere. The reaction was quenched by the addition of water (5 mL) at room temperature. The resulting mixture was extracted with EtOAc (2×10 mL). The combined organic layers were washed with brine (5 mL), dried over anhydrous Na₂SO₄. After filtration, the filtrate was concentrated under reduced pressure. The residue was purified by silica gel column chromatography, eluted with PE/EtOAc/EtOH=(4/3/1), the crude product was purified by reverse flash chromatography with the following conditions: Column: SunFire Prep C18 OBD Column, 19×150 mm Sum 10 nm; Mobile Phase A: water (0.1% FA), Mobile Phase B: CH₃CN; Flow rate:20 mL/min; Gradient:35 B to 70 B in 4.3 min; 210/254 nm. This resulted in (S)-N-(3-(2-(((1R,3S)-3-hydroxy-3-methylcyclobutyl)amino)-6-morpholinopyridin-4-yl)-4-methylphenyl)-3-(2,2,2-trifluoroethyl)pyrrolidine-1-carboxamide (114 mg, 56%) as an off-white solid. MS ESI calculated for C₂₈H₃₆F₃N₅O₃[M+H]⁺, 548.28, found 548.15. ¹H NMR (300 MHz, DMSO-d₆) δ 8.13-8.11 (m, 1H), 7.43-7.40 (m, 1H), 7.33 (s, 1H), 7.10-7.08 (m, 1H), 6.42 (m, 1H), 5.77 (s, 1H), 5.64 (s, 1H), 4.83-4.23 (m, 1H), 4.45-4.05 (m, 1H), 3.80-3.67 (m, 5H), 3.57-3.55 (m, 1H), 3.52-3.38 (m, 5H), 3.10-2.99 (m, 1H), 2.46-2.32 (m, 5H), 2.29-2.08 (m, 4H), 1.94-1.84 (m, 2H), 1.72-1.62 (m, 1H), 1.27-1.06 (m, 3H). ¹⁹F NMR (282 MHz, DMSO-d₆) δ-63.35-63.42(3F).

### Example 36: (S)-N-(3-(2-(((1S,4R)-4-hydroxy-4-methylcyclohexyl)amino)-6-morpholinopyridin-4-yl)-4-methylphenyl)-3-(2,2,2-trifluoroethyl)pyrrolidine-1-carboxamide

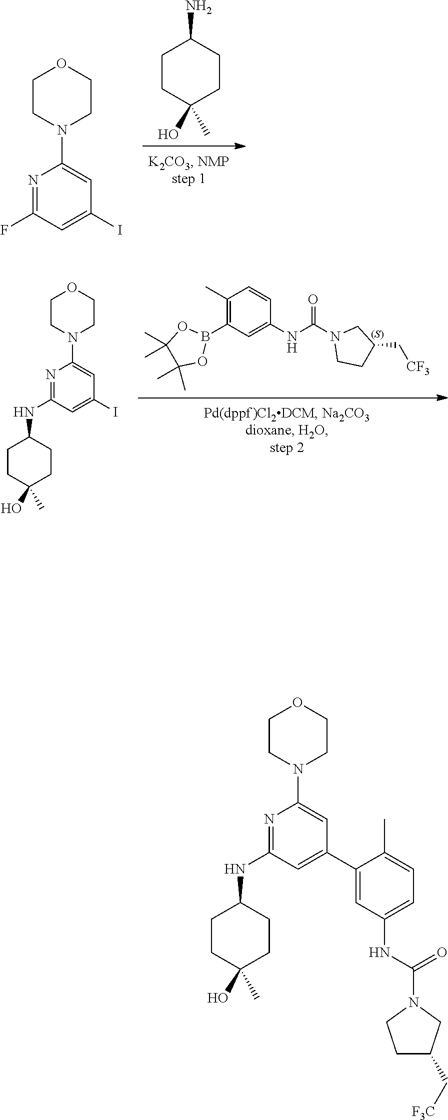

Preparation 36A: (1S,4S)-4-((4-iodo-6-morpholinopyridin-2-yl)amino)-1-methylcyclohexan-1-ol

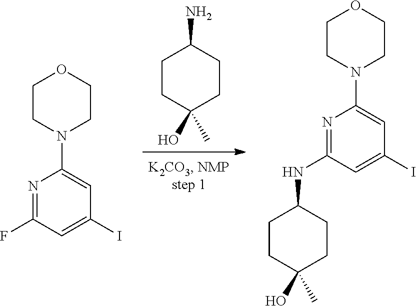

To a stirred mixture of 4-(6-fluoro-4-iodopyridin-2-yl)morpholine (300 mg, 0.974 mmol) and K₂CO₃ (407 mg, 2.921 mmol) in NMP (3 mL) was added (cis)-4-amino-1-methylcyclohexan-1-ol (189 mg, 1.461 mmol) at room temperature. The resulting mixture was stirred for 16 h at 150° C. The mixture was allowed to cool down to room temperature. The residue was purified by reverse flash chromatography with the following conditions: column, C18 silica gel; mobile phase, CH₃CN in water, 30% to 70% gradient in 20 min; detector, UV 254 nm to afford (1S,4S)-4-((4-iodo-6-morpholinopyridin-2-yl)amino)-1-methylcyclohexan-1-ol (290 mg, 71%) as a light brown solid. MS ESI calculated for C₁₆H₂₄IN₃O₂ 418.09, found 417.95. ¹H NMR (300 MHz, chloroform-d) δ 6.26 (s, 1H), 6.16 (s, 1H), 4.24 (d, J=8.0 Hz, 1H), 3.84-3.75 (m, 4H), 3.45-3.42 (m, 5H), 2.00-1.40 (m, 8H), 1.29 (s, 3H), 1.13 (s, 1H).

### Example 36: (S)-N-(3-(2-(((1S,4R)-4-hydroxy-4-methylcyclohexyl)amino)-6-morpholinopyridin-4-yl)-4-methylphenyl)-3-(2,2,2-trifluoroethyl)pyrrolidine-1-carboxamide

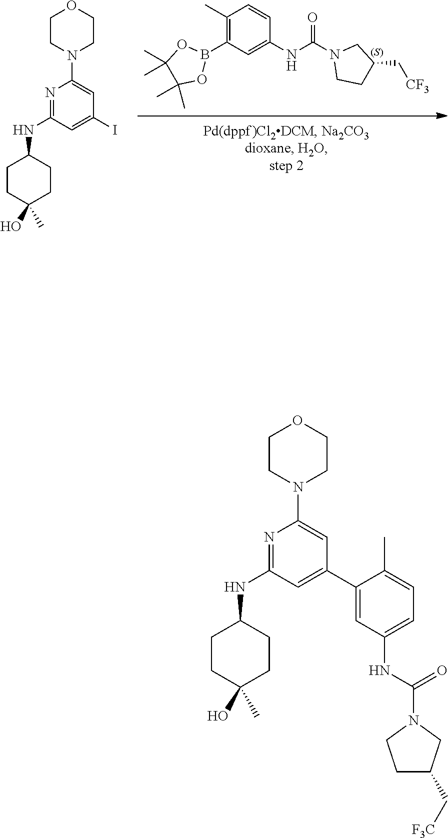

To a stirred mixture of (1S,4S)-4-((4-iodo-6-morpholinopyridin-2-yl)amino)-1-methylcyclohexan-1-ol (150 mg, 0.359 mmol) and (3S)-N-[4-methyl-3-(4,4,5-trimethyl-1,3,2-dioxaborolan-2-yl)phenyl]-3-(2,2,2-trifluoroethyl)pyrrolidine-1-carboxamide (143 mg, 0.359 mmol) in 1,4-dioxane (4 mL) and H₂O (1 mL) were added Na₂CO₃ (114 mg, 1.078 mmol) and Pd(dppf)Cl₂·DCM (29 mg, 0.036 mmol) at 70° C. under nitrogen atmosphere. The reaction was quenched by the addition of water (20 mL) at room temperature. The resulting mixture was extracted with EtOAc (3×50 mL). The combined organic layers were washed with brine (2×5 mL), dried over anhydrous Na₂SO₄. After filtration, the filtrate was concentrated under reduced pressure. The residue was purified by silica gel column chromatography, eluted with PE/EtOAc (1:1) to afford (S)-N-(3-(2-(((1S,4R)-4-hydroxy-4-methylcyclohexyl)amino)-6-morpholinopyridin-4-yl)-4-methylphenyl)-3-(2,2,2-trifluoroethyl)pyrrolidine-1-carboxamide (98 mg, 47%) as a light green solid. MS ESI calculated for C₃₀H₄₀F₃N₅O₃, 576.31 found 576.20. ¹H NMR (300 MHz, DMSO-d₆) δ 8.12 (s, 1H), 7.41 (dd, J=8.2, 2.3 Hz, 1H), 7.33 (d, J=2.3 Hz, 1H), 7.09 (d, J=8.4 Hz, 1H), 6.07 (d, J=7.4 Hz, 1H), 5.73 (s, 2H), 4.02 (s, 1H), 3.68 (dd, J=5.9, 3.7 Hz, 5H), 3.52 (td, J=9.4, 8.3, 5.3 Hz, 2H), 3.37 (s, 3H), 3.02 (t, J=9.4 Hz, 1H), 2.44 (dd, J=12.6, 8.1 Hz, 3H), 2.16 (s, 3H), 2.09 (d, J=11.9 Hz, 1H), 1.69 (t, J=7.9 Hz, 3H), 1.56 (p, J=9.9, 8.8 Hz, 5H), 1.43-1.28 (m, 3H), 1.24 (s, 1H), 1.12 (s, 3H).

### Example 37: (S)-N-(3-(2-(((1R,4S)-4-hydroxy-4-methylcyclohexyl)amino)-6-morpholinopyridin-4-yl)-4-methylphenyl)-3-(2,2,2-trifluoroethyl)pyrrolidine-1-carboxamide

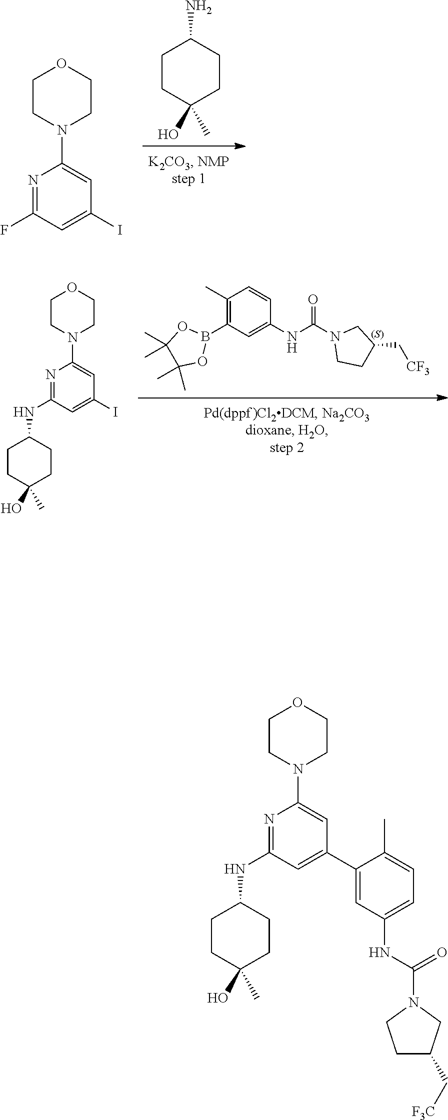

Preparation 37A: (1R,4R)-4-((4-iodo-6-morpholinopyridin-2-yl)amino)-1-methylcyclohexan-1-ol

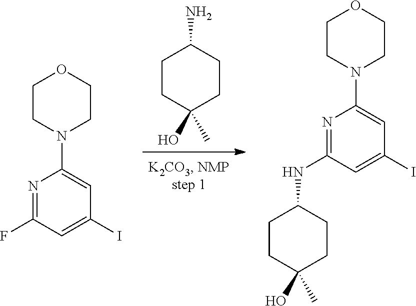

To a stirred mixture of 4-(6-fluoro-4-iodopyridin-2-yl)morpholine (300 mg, 0.974 mmol) and K₂CO₃ (404 mg, 2.921 mmol) in NMP (3 mL) was added (1R,4R)-4-amino-1-methylcyclohexan-1-ol (189 mg, 1.461 mmol) at room temperature. The resulting mixture was stirred for 16 h at 150° C. The mixture was allowed to cool down to room temperature. The residue was purified by reverse flash chromatography with the following conditions: column, C18 silica gel; mobile phase, CH₃CN in water, 30% to 70% gradient in 20 min; detector, UV 254 nm to afford (1R,4R)-4-((4-iodo-6-morpholinopyridin-2-yl)amino)-1-methylcyclohexan-1-ol (320 mg, 79%) as a light brown solid. MS ESI calculated for C₁₆H₂₄IN₃O₂[M+H]⁺ 418.09 found 418.00. ¹H NMR (300 MHz, chloroform-d) δ 6.28 (s, 1H), 6.18 (s, 1H), 4.27 (s, 1H), 3.84-3.75 (m, 4H), 3.63-3.39 (m, 5H), 2.02-1.36 (m, 8H), 1.31 (s, 3H), 1.27 (s, 1H).

### Example 37: (S)-N-(3-(2-(((1R,4S)-4-hydroxy-4-methylcyclohexyl)amino)-6-morpholinopyridin-4-yl)-4-methylphenyl)-3-(2,2,2-trifluoroethyl)pyrrolidine-1-carboxamide

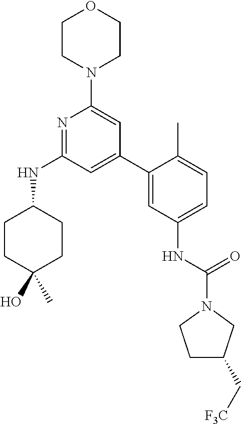 [SMILES: Cc1ccc(NC(=O)N2CC[C@@H](CC(F)(F)F)C2)cc1-c1cc(N[C@H]2CC[C@](C)(O)CC2)nc(N2CCOCC2)c1]

A mixture of (1R,4R)-4-((4-iodo-6-morpholinopyridin-2-yl)amino)-1-methylcyclohexan-1-ol (220 mg, 0.527 mmol), (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (217 mg, 0.527 mmol), 1,4-dioxane (5.3 mL), H₂O (1.3 mL), Na₂CO₃ (168 mg, 1.582 mmol) and Pd(dppf)Cl₂·DCM (43 mg, 0.053 mmol) was stirred for 2 h at 70° C. under nitrogen atmosphere. The reaction was quenched by the addition of water (20 mL) at room temperature. The resulting mixture was extracted with EtOAc (3×50 mL). The combined organic layers were washed with brine (2×5 mL), dried over anhydrous Na₂SO₄. After filtration, the filtrate was concentrated under reduced pressure. The residue was purified by silica gel column chromatography, eluted with PE/EtOAc (1/1) to afford (S)-N-(3-(2-(((1R,4S)-4-hydroxy-4-methylcyclohexyl)amino)-6-morpholinopyridin-4-yl)-4-methylphenyl)-3-(2,2,2-trifluoroethyl)pyrrolidine-1-carboxamide (153 mg, 50%) as a light green solid. MS ESI calculated for C₃₀H₄₀F₃N₅O₃ [M+H]⁺ 576.31, found 576.15. ¹H NMR (300 MHz, DMSO-d₆) δ 8.12 (s, 1H), 7.41 (dd, J=8.3, 2.3 Hz, 1H), 7.34 (d, J=2.3 Hz, 1H), 7.09 (d, J=8.4 Hz, 1H), 6.08 (d, J=7.3 Hz, 1H), 5.74 (d, J=4.4 Hz, 2H), 4.20 (s, 1H), 3.67 (q, J=8.3, 6.4 Hz, 6H), 3.57-3.50 (m, 1H), 3.44-3.27 (m, 5H), 3.02 (t, J=9.4 Hz, 1H), 2.47 (d, J=19.5 Hz, 3H), 2.18-2.16 (m, 4H), 1.93-1.81 (m, 2H), 1.75-1.53 (m, 3H), 1.41 (h, J=10.4 Hz, 4H), 1.14 (s, 3H).

### Example 38: (3S)-N-[3-[2-(3-hydroxy-3-methylazetidin-1-yl)-6-(morpholin-4-yl)pyridin-4-yl]-4-methylphenyl]-3-(2,2,2-trifluoroethyl)pyrrolidine-1-carboxamide

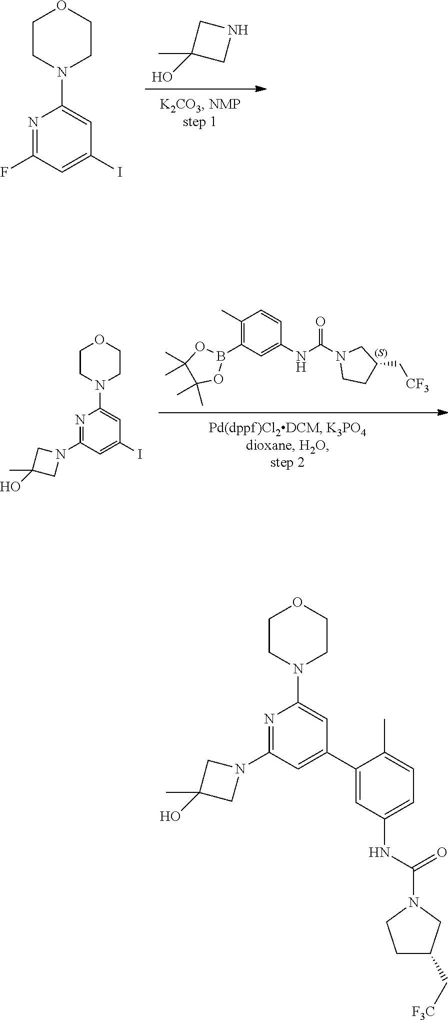

Preparation 38A: 1-[4-iodo-6-(morpholin-4-yl)pyridin-2-yl]-3-methylazetidin-3-ol

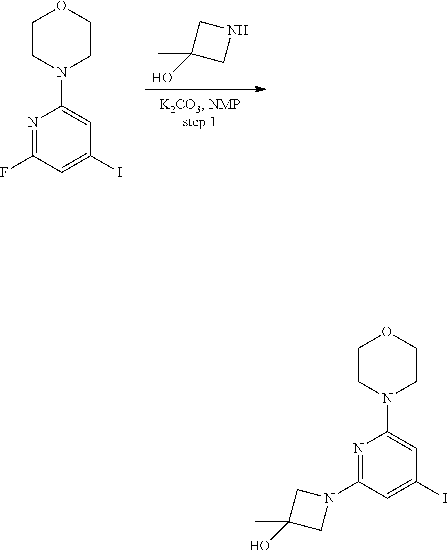

To a stirred solution/mixture of 4-(6-fluoro-4-iodopyridin-2-yl)morpholine (300 mg, 0.974 mmol) and 3-methylazetidin-3-ol (127 mg, 1.461 mmol) in NMP (3 mL) was added K₂CO₃ (404 mg, 3.00 equiv) at room temperature. The resulting mixture was stirred for 16 h at 150° C. The residue was purified by reverse flash chromatography with the following conditions: column, C18 silica gel; mobile phase, MeOH in water, 10% to 50% gradient in 30 min; detector, UV 254 nm. to afford 1-[4-iodo-6-(morpholin-4-yl)pyridin-2-yl]-3-methylazetidin-3-ol (270 mg, 74%) as an off-white solid. MS ESI calculated for C₁₃H₁₈IN₃O₂, [M+H]⁺, 376.21, found 376.00. ¹H NMR (300 MHz, chloroform-d) δ 6.32 (s, 1H), 6.10 (s, 1H), 3.89 (q, J=8.6 Hz, 4H), 3.83-3.75 (m, 4H), 3.50-3.41 (m, 4H), 1.60 (s, 3H)

### Example 38: (3S)-N-[3-[2-(3-hydroxy-3-methylazetidin-1-yl)-6-(morpholin-4-yl)pyridin-4-yl]-4-methylphenyl]-3-(2,2,2-trifluoroethyl)pyrrolidine-1-carboxamide

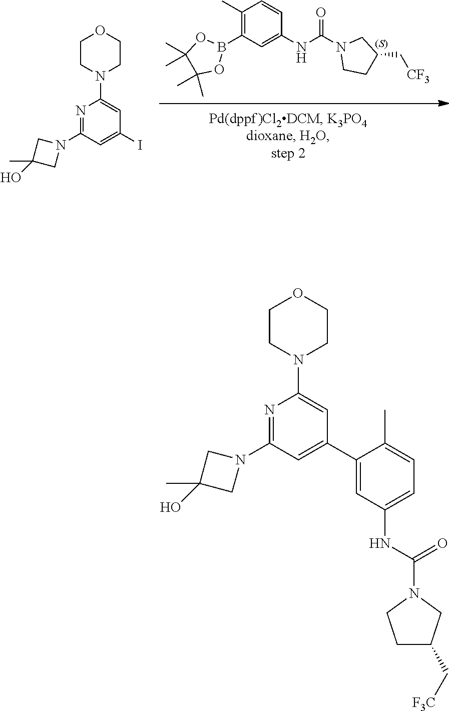

A mixture of 1-[4-iodo-6-(morpholin-4-yl)pyridin-2-yl]-3-methylazetidin-3-ol (150 mg, 0.400 mmol) and (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (181 mg, 0.440 mmol), 1,4-dioxane (1.6 mL), H₂O (0.4 mL), K₃PO₄ (255 mg, 1.199 mmol) and Pd(dppf)Cl₂·DCM (43 mg, 0.053 mmol) at room temperature under N₂ atmosphere. The resulting mixture was stirred for 1 h at 80° C. under N₂ atmosphere. The resulting mixture was concentrated under reduced pressure. The residue was purified by silica gel column chromatography, eluted with PE/EtOAc (1/1) to afford (3S)-N-[3-[2-(3-hydroxy-3-methylazetidin-1-yl)-6-(morpholin-4-yl)pyridin-4-yl]-4-methylphenyl]-3-(2,2,2-trifluoroethyl)pyrrolidine-1-carboxamide (79 mg, 37%) as an off-white solid. MS ESI calculated for C₂₇H₃₄F₃N₅O₃ [M+H]⁺, 534.26, found 534.15. ¹H NMR (400 MHz, DMSO-d₆) δ 8.12 (s, 1H), 7.43 (dd, J=8.3, 2.3 Hz, 1H), 7.33 (d, J=2.4 Hz, 1H), 7.10 (d, J=8.4 Hz, 1H), 5.93 (d, J=1.0 Hz, 1H), 5.61 (d, J=0.9 Hz, 1H), 5.49 (s, 1H), 3.80-3.62 (m, 9H), 3.52 (ddd, J=10.4, 8.3, 2.3 Hz, 1H), 3.40 (t, J=4.9 Hz, 4H), 3.32 (d, J=2.5 Hz, 1H), 3.02 (t, J=9.5 Hz, 1H), 2.45 (d, J=11.3 Hz, 1H), 2.41 (s, 2H), 2.15 (s, 3H), 2.12-2.00 (m, 1H), 1.65 (p, J=9.9 Hz, 1H), 1.43 (s, 3H). ¹⁹F NMR (376 MHz, DMSO-d₆) δ-63.12.

### Example 39: (3S)-N-[3-[2-(isopropylamino)-6-(morpholin-4-yl)pyridin-4-yl]-4-methylphenyl]-3-(2,2,2-trifluoroethyl)pyrrolidine-1-carboxamide

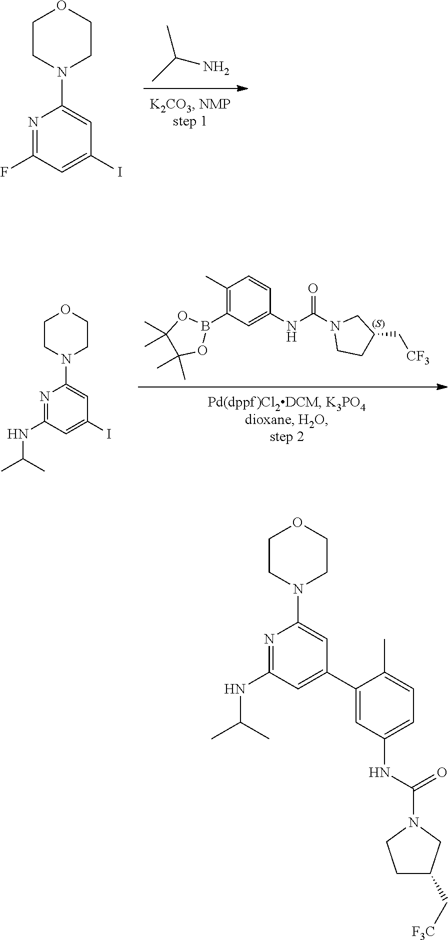

Preparation 39A: 4-iodo-N-isopropyl-6-(morpholin-4-yl)pyridin-2-amine

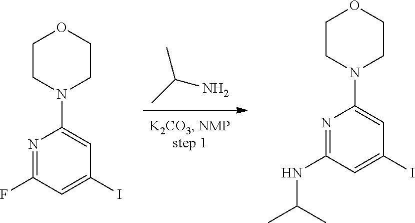

A mixture of 4-(6-fluoro-4-iodopyridin-2-yl)morpholine (300 mg, 0.974 mmol), isopropylamine (86 mg, 1.461 mmol), NMP (3 mL) and K₂CO₃ (404 mg, 2.921 mmol) was stirred for 16 h at 150° C. The resulting mixture was concentrated under reduced pressure. The residue was purified by silica gel column chromatography, eluted with PE/EtOAc (0-50%) to afford 4-iodo-N-isopropyl-6-(morpholin-4-yl)pyridin-2-amine (210 mg, 62%) as a white solid. MS ESI calculated for C₁₂H₁₈IN₃O [M+H]⁺, 348.05, found 348.10. ¹H NMR (300 MHz, chloroform-d) δ 6.27 (s, 1H), 6.16 (s, 1H), 4.17 (d, J=7.7 Hz, 1H), 3.92-3.74 (m, 5H), 3.48-3.39 (m, 4H), 1.22 (d, J=6.4 Hz, 6H).

### Example 39: (3S)-N-[3-[2-(isopropylamino)-6-(morpholin-4-yl)pyridin-4-yl]-4-methylphenyl]-3-(2,2,2-trifluoroethyl)pyrrolidine-1-carboxamide

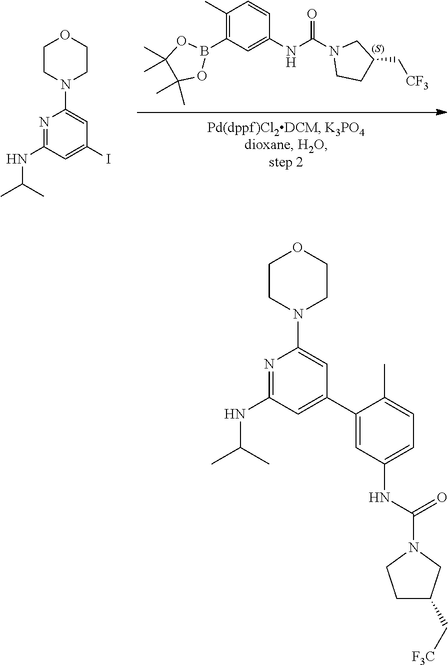

A mixture of 4-iodo-N-isopropyl-6-(morpholin-4-yl)pyridin-2-amine (150 mg, 0.432 mmol), (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (196 mg, 0.475 mmol), 1,4-dioxane (2 mL), H₂O (0.5 mL), K₃PO₄ (275 mg, 1.296 mmol) and Pd(dppf)Cl₂·DCM (43 mg, 0.053 mmol) was stirred for 1 h at 80° C. under N₂ atmosphere. The resulting mixture was concentrated under reduced pressure. The residue was purified by silica gel column chromatography, eluted with PE/EtOAc (1/1) to afford (3S)-N-[3-[2-(isopropylamino)-6-(morpholin-4-yl)pyridin-4-yl]-4-methylphenyl]-3-(2,2,2-trifluoroethyl)pyrrolidine-1-carboxamide (86 mg, 40%)as an off-white solid. MS ESI calculated for C₂₆H₃₄F₃N₅O₂[M+H]⁺, 506.27, found 506.15. ¹H NMR (400 MHz, DMSO-d₆) δ 8.13 (d, J=17.1 Hz, 1H), 7.41 (dd, J=8.3, 2.3 Hz, 1H), 7.33 (d, J=2.4 Hz, 1H), 7.09 (d, J=8.4 Hz, 1H), 6.05 (d, J=7.5 Hz, 1H), 5.72 (dd, J=8.5, 1.0 Hz, 2H), 3.96 (h, J=6.6 Hz, 1H), 3.71-3.62 (m, 5H), 3.57-3.47 (m, 1H), 3.36-3.30 (m, 5H), 3.02 (t, J=9.4 Hz, 1H), 2.50-2.42 (m, 1H), 2.41-2.39 (m, 2H), 2.16 (s, 3H), 2.08 (s, 1H), 1.67(q, J=10.5, 10.1 Hz, 1H), 1.15 (d, J=6.5 Hz, 6H).

### Example 40: (3S)-N-[3-[2-(tert-butylamino)-6-(morpholin-4-yl)pyridin-4-yl]-4-methylphenyl]-3-(2,2,2-trifluoroethyl)pyrrolidine-1-carboxamide

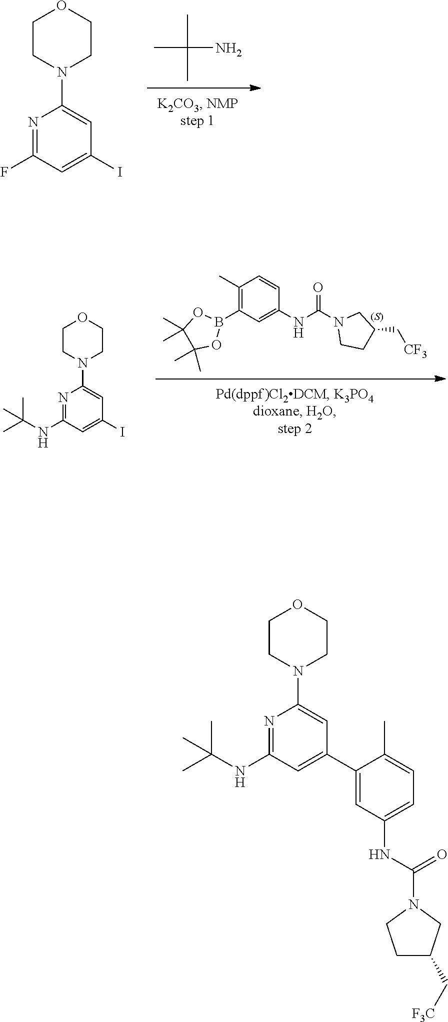

Preparation 40A: N-tert-butyl-4-iodo-6-(morpholin-4-yl)pyridin-2-amine

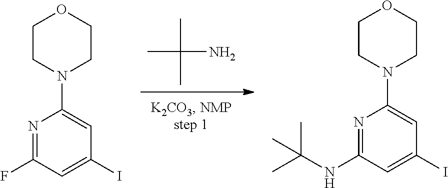

A mixture of 4-(6-fluoro-4-iodopyridin-2-yl)morpholine (300 mg, 0.974 mmol), 3-methylazetidin-3-ol (127 mg, 1.461 mmol), NMP (3 mL) and K₂CO₃ (404 mg, 2.921 mmol) was stirred for 16 h at 150° C. The reaction was monitored by LCMS. The residue was purified by silica gel column chromatography, eluted with PE/EtOAc (1/1) to afford N-tert-butyl-4-iodo-6-(morpholin-4-yl)pyridin-2-amine (120 mg, 34%) as a white solid. MS ESI calculated for C₁₃H₂₀IN₃O [M+H]⁺, 362.07, found 362.05. ¹H NMR (400 MHz, DMSO-d₆) δ 6.28 (d, J=1.0 Hz, 1H), 6.19-6.11 (m, 2H), 3.66 (t, J=4.9 Hz, 4H), 3.44-3.29 (m, 4H), 1.34 (s, 9H).

### Example 40: (3S)-N-[3-[2-(tert-butylamino)-6-(morpholin-4-yl)pyridin-4-yl]-4-methylphenyl]-3-(2,2,2-trifluoroethyl)pyrrolidine-1-carboxamide

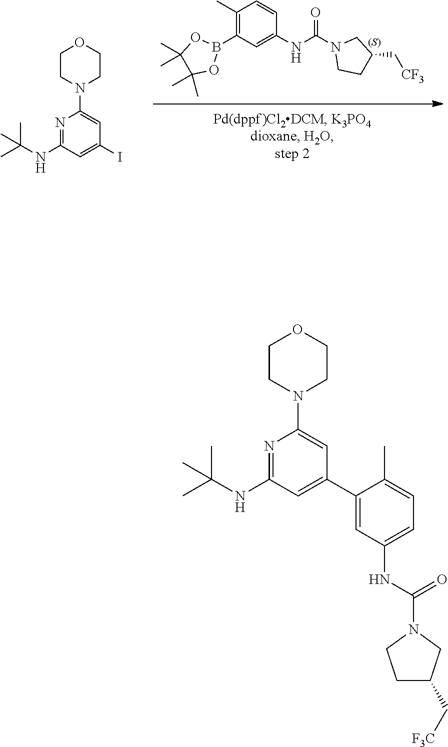

To a stirred mixture of N-tert-butyl-4-iodo-6-(morpholin-4-yl)pyridin-2-amine (100 mg, 0.277 mmol) and (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (126 mg, 0.305 mmol) in dioxane (1 mL) H₂O (0.2 mL), were added K₃PO₄ (176 mg, 0.831 mmol) and Pd(dppf)Cl₂·CH₂Cl₂ (23 mg, 0.028 mmol) in portions at room temperature under N₂ atmosphere. The resulting mixture was stirred for 1 h at 80° C. under N₂ atmosphere. The resulting mixture was concentrated under reduced pressure. The residue was purified by silica gel column chromatography, eluted with PE/EtOAc (1:1). The residue was purified by reverse flash chromatography with the following conditions: Column: XBridge Prep Phenyl OBD Column, 19×150 mm 5 μm 13 nm; Mobile Phase A:Water(10 mmol/1 NH₄HCO₃), Mobile Phase B: CH₃CN; Flow rate:20 mL/min; Gradient:30 B to 70 B in 4.2 min; 254/210 nm. The fractions contained desired product were combined and concentrated to afford (3S)-N-[3-[2-(tert-butylamino)-6-(morpholin-4-yl)pyridin-4-yl]-4-methylphenyl]-3-(2,2,2-trifluoroethyl)pyrrolidine-1-carboxamide (86 mg, 60%) as a white soild. MS ESI calculated for C₂₇H₃₆F₃N₅O₂[M+H]⁺, 520.28, found 520.15. ¹H NMR (400 MHz, DMSO-d₆) δ 8.10 (s, 1H), 7.40 (dd, J=8.2, 2.3 Hz, 1H), 7.34 (d, J=2.3 Hz, 1H), 7.09 (d, J=8.3 Hz, 1H), 5.98 (s, 1H), 5.81 (s, 1H), 5.73 (s, 1H), 3.69 (q, J=8.0, 6.3 Hz, 5H), 3.57-3.48 (m, 1H), 3.37 (s, 1H), 3.32 (s, 1H), 3.02 (t, J=9.4 Hz, 1H), 2.45 (d, J=11.3 Hz, 1H), 2.41 (s, 2H), 2.16 (s, 3H), 2.12-2.05 (m, 1H), 1.70-1.61 (m, 1H), 1.40 (s, 9H). ¹⁹F NMR (376 MHz, DMSO-d₆) δ-63.37 (3F).

### Example 41: (3S)-N-(3-[2-[(1-hydroxy-2-methylpropan-2-yl)amino]-6-(morpholin-4-yl)pyridin-4-yl]-4-methylphenyl)-3-(2,2,2-trifluoroethyl)pyrrolidine-1-carboxamide

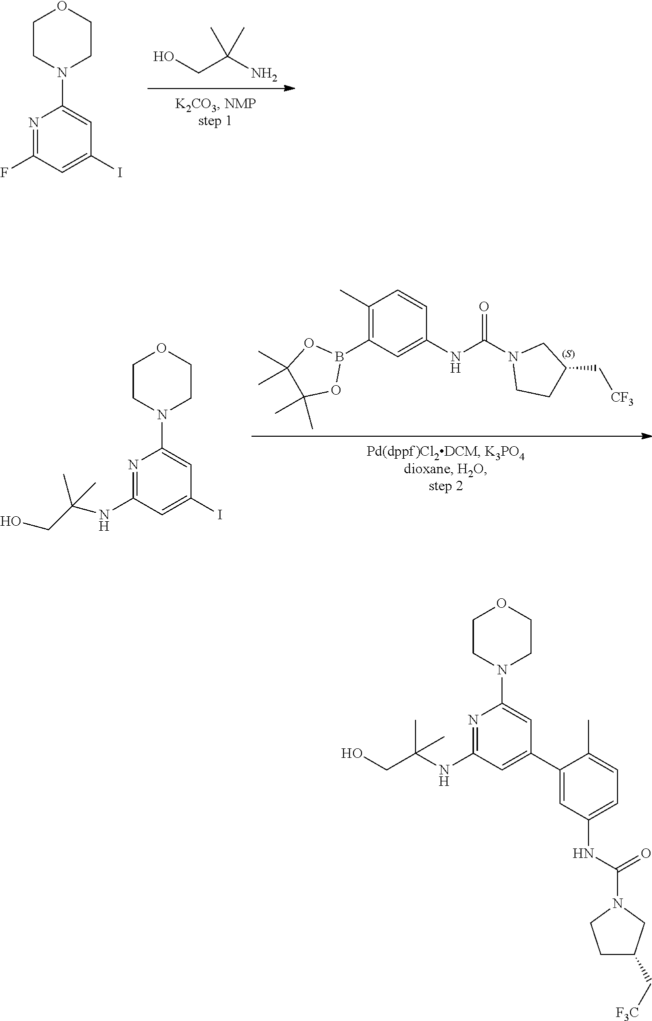

Preparation 41A: 2-[[4-iodo-6-(morpholin-4-yl)pyridin-2-yl]amino]-2-methylpropan-1-ol

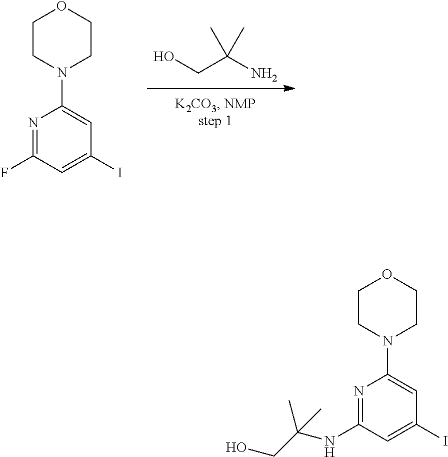

To a stirred solution of 4-(6-fluoro-4-iodopyridin-2-yl)morpholine (300 mg, 0.974 mmol) and 2-amino-2-methyl-1-propanol (130 mg, 1.461 mmol) in NMP (3 mL) was added K₂CO₃ (404 mg, 2.922 mmol) in portions at room temperature. The resulting mixture was stirred for 16 h at 150° C. The residue was purified by silica gel column chromatography, eluted with PE/EtOAc (0-50%) to afford 2-[[4-iodo-6-(morpholin-4-yl)pyridin-2-yl]amino]-2-methylpropan-1-ol (160 mg, 44%) as a light blue solid. MS ESI calculated for C₁₃H₂AlN₃O₂[M+H]⁺, 378.06, found 378.10. ¹H NMR (400 MHz, DMSO-d₆) δ 6.67 (s, 1H), 6.49 (s, 1H), 3.87 (s, 2H), 3.67 (t, J=4.7 Hz, 4H), 3.42 (t, J=4.8 Hz, 4H), 1.57 (s, 2H), 1.05 (s, 6H).

### Example 41: (3S)-N-(3-[2-[(1-hydroxy-2-methylpropan-2-yl)amino]-6-(morpholin-4-yl)pyridin-4-yl]-4-methylphenyl)-3-(2,2,2-trifluoroethyl)pyrrolidine-1-carboxamide

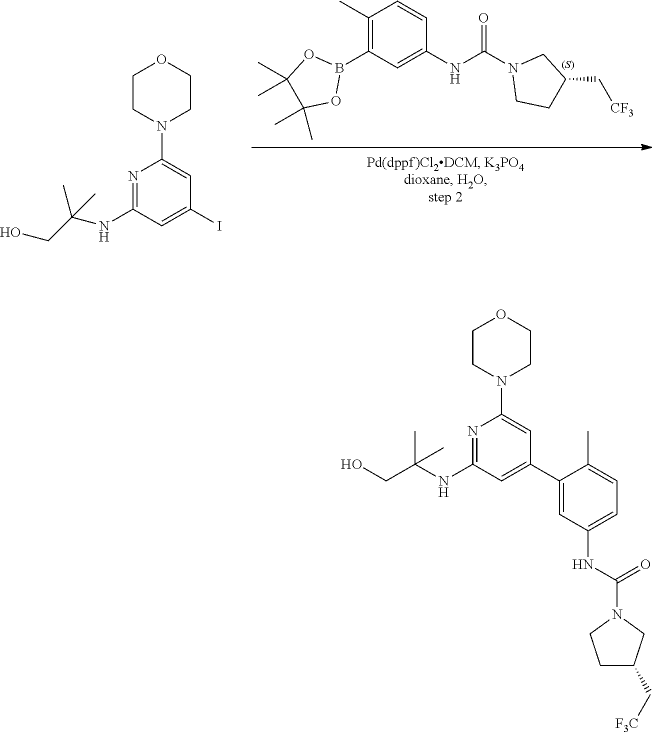

To a stirred mixture of 2-[[4-iodo-6-(morpholin-4-yl)pyridin-2-yl]amino]-2-methylpropan-1-ol (100 mg, 0.265 mmol) and (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (120 mg, 0.292 mmol) in dioxane (2 mL), H₂O (0.5 mL), were added K₃PO₄ (169 mg, 0.795 mmol) and Pd(dppf)Cl₂·CH₂Cl₂ (22 mg, 0.027 mmol) in portions at room temperature under N₂ atmosphere. The resulting mixture was stirred for 1 h at 80° C. under N₂ atmosphere. The resulting mixture was concentrated under reduced pressure. The residue was purified by silica gel column chromatography, eluted with EA. The residue was purified by reverse flash chromatography with the following conditions: Column: XBridge Prep Phenyl OBD Column, 19×150 mm Sum 13 nm; Mobile Phase A:Water(10 mmol/1 NH₄HCO₃), Mobile Phase B: CH₃CN; Flow rate:20 mL/min; Gradient:40 B to 75 B in 4.3 min; 254/210 nm. The fractions contained desired product were combined and concentrated to afford (3S)-N-(3-[2-[(1-hydroxy-2-methylpropan-2-yl)amino]-6-(morpholin-4-yl)pyridin-4-yl]-4-methylphenyl)-3-(2,2,2-trifluoroethyl)pyrrolidine-1-carboxamide (31 mg, 22%) as a light blue soild. MS ESI calculated for C₂₇H₃₆F₃N₅O₂[M+H]⁺, 536.28, found 536.15. ¹H NMR (400 MHz, DMSO-d₆) δ 8.11 (s, 1H), 7.40 (dd, J=8.2, 2.4 Hz, 1H), 7.35 (d, J=2.3 Hz, 1H), 7.09 (d, J=8.3 Hz, 1H), 5.82 (d, J=14.0 Hz, 2H), 5.75 (s, 1H), 4.90 (t, J=5.7 Hz, 1H), 3.73-3.62 (m, 5H), 3.58-3.48 (m, 3H), 3.32 (dd, J=16.9, 10.3 Hz, 3H), 3.02 (t, J=9.4 Hz, 1H), 2.51-2.34 (m, 2H), 2.17 (s, 3H), 2.08 (t, J=5.8 Hz, 1H), 1.67 (q, J=10.0 Hz, 1H), 1.32 (s, 6H). ¹⁹F-NMR (376 MHz, DMSO-d₆) δ-63.37 (3F).

### Example 42: (3S)-N-[4-methyl-3-[2-(morpholin-4-yl)-6-(oxetan-3-ylamino)pyridin-4-yl]phenyl]-3-(2,2,2-trifluoroethyl)pyrrolidine-1-carboxamide

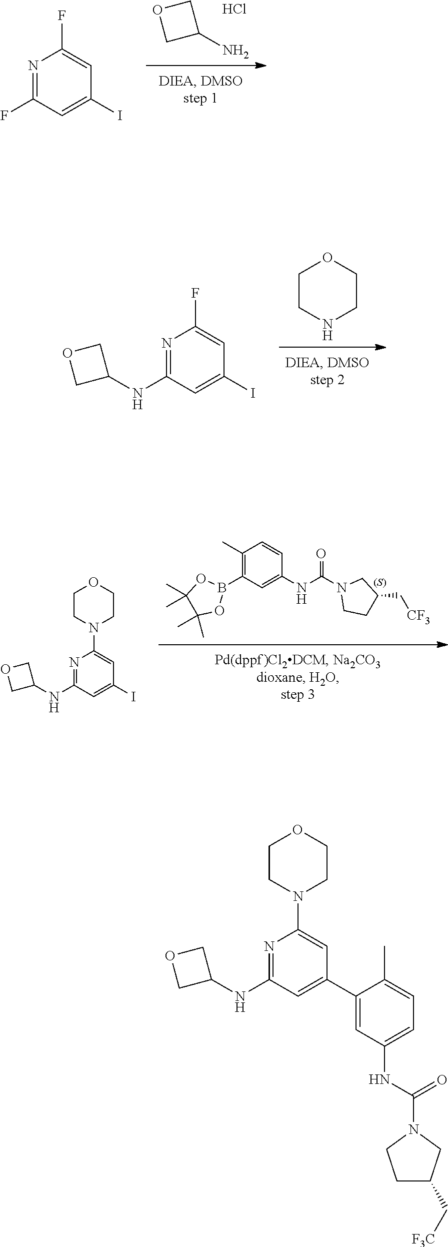

Preparation 42A: 6-fluoro-4-iodo-N-(oxetan-3-yl)pyridin-2-amine

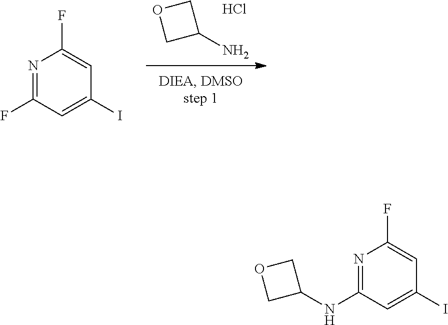 [SMILES: Fc1cc(I)cc(F)n1 ; NC1COC1 ; Cl ; Fc1cc(I)cc(NC2COC2)n1]

To a stirred mixture of 2,6-difluoro-4-iodopyridine (300 mg, 1.245 mmol) and oxetan-3-amine hydrochloride (150 mg, 1.369 mmol), DIEA (354 mg, 2.739 mmol) in DMSO (4 mL) at room temperature under nitrogen atmosphere. The resulting mixture was stirred for 3 h at 70° C. under nitrogen atmosphere. No further workup and used in next step directly. MS ESI calculated for C₈H₈FIN₂O [M+H]⁺, 294.97, found 295.05.

Preparation 42B: 4-iodo-6-(morpholin-4-yl)-N-(oxetan-3-yl)pyridin-2-amine

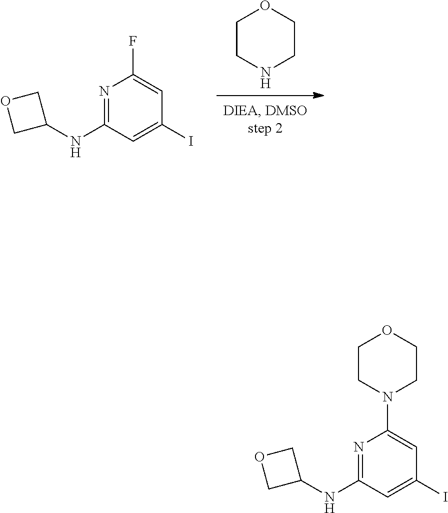 [SMILES: Fc1cc(I)cc(NC2COC2)n1 ; C1COCCN1 ; Ic1cc(NC2COC2)nc(N2CCOCC2)c1]

To a stirred mixture of 6-fluoro-4-iodo-N-(oxetan-3-yl)pyridin-2-amine (300 mg, 1.020 mmol) and morpholine (444 mg, 5.101 mmol), DIEA (145 mg, 1.122 mmol) in DMSO (4.5 mL) at room temperature under nitrogen atmosphere. The resulting mixture was stirred for 16 h at 70° C. under nitrogen atmosphere. The resulting mixture was diluted with water (50 mL). The resulting mixture was extracted with EtOAc (3×60 mL). The combined organic layers were washed with brine (2×50 mL), dried over anhydrous Na₂SO₄ After filtration, the filtrate was concentrated under reduced pressure. The residue was purified by silica gel column chromatography, eluted with PE/EtOAc/EtOH=(4/3/1) to afford 4-iodo-6-(morpholin-4-yl)-N-(oxetan-3-yl)pyridin-2-amine (395 mg, 90%) as an off-white solid. MS ESI calculated for C₁₂H₁₆IN₃O₂[M+H]⁺, 362.03, found 362.00. ¹H NMR (400 MHz, chloroform-d) δ 6.35-6.34 (m, 1H), 6.15 (s, 1H), 4.99-4.95 (m, 2H), 4.57-4.55 (m, 2H), 3.81-3.87 (m, 4H), 3.86-3.43 (m, 5H).

### Example 42: (3S)-N-[4-methyl-3-[2-(morpholin-4-yl)-6-(oxetan-3-ylamino)pyridin-4-yl]phenyl]-3-(2,2,2-trifluoroethyl)pyrrolidine-1-carboxamide

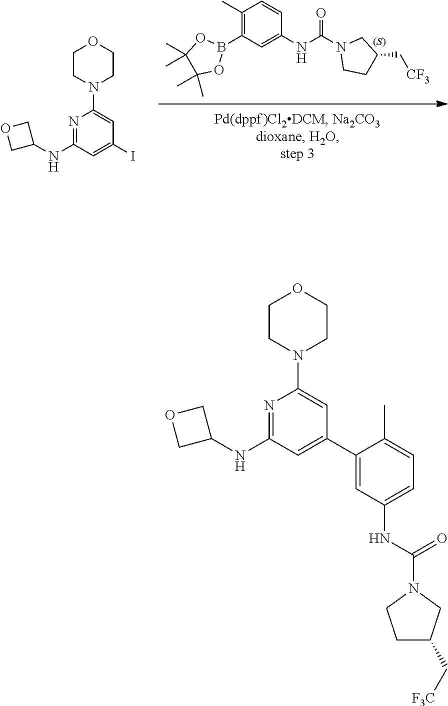

A mixture of 4-iodo-6-(morpholin-4-yl)-N-(oxetan-3-yl)pyridin-2-amine (200 mg, 0.554 mmol), (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (228 mg, 0.554 mmol), Na₂CO₃ (176 mg, 1.661 mmol), dioxane (1.6 mL), H₂O (0.4 mL) and Pd(dppf)Cl₂·CH₂Cl₂ (45 mg, 0.055 mmol) was stirred for 16 h at 60° C. The resulting mixture was diluted with H₂O (20 mL). The resulting mixture was extracted with EtOAc (3×20 mL). The combined organic layers were washed with brine (2×15 mL), dried over anhydrous Na₂SO₄. After filtration, the filtrate was concentrated under reduced pressure. The residue was purified by silica gel column chromatography, eluted with PE/EtOAc/EtOH=(4/3/1) followed by Prep-HPLC with the following conditions (Column: SunFire Prep C18 OBD Column, 19×150 mm Sum 10 nm; Mobile Phase A: water (0.1% FA), Mobile Phase B: CH₃CN; Flow rate:20 mL/min; Gradient:20 B to 50 B in 4.3 min; 210/254 nm to afford (3S)-N-[4-methyl-3-[2-(morpholin-4-yl)-6-(oxetan-3-ylamino)pyridin-4-yl]phenyl]-3-(2,2,2-trifluoroethyl)pyrrolidine-1-carboxamide (160 mg, 55%) as a white solid. MS ESI calculated for C₂₆H₃₂F₃N₅O₃[M+H]⁺, 520.25, found 520.15. ¹H NMR (400 MHz, DMSO-d₆) δ 8.13 (s, 1H), 7.54-7.34 (m, 2H), 7.11-7.01 (m, 2H), 5.83-5.74 (m, 2H), 4.87-4.77 (m, 3H), 4.48-4.32 (m, 2H), 3.76-3.60 (m, 6H), 3.54-3.21 (m, 5H), 3.04-2.99 (m, 1H), 2.45-2.41 (m, 3H), 2.34-2.07 (m, 4H), 1.68-1.63 (m, 1H). ¹⁹F NMR (376 MHz, DMSO-d₆) δ-63.36 (3F).

### Example 43: (3S)-N-[4-methyl-3-[2-(morpholin-4-yl)-6-[(3R)-oxolan-3-ylamino]pyridin-4-yl]phenyl]-3-(2,2,2-trifluoroethyl)pyrrolidine-1-carboxamide

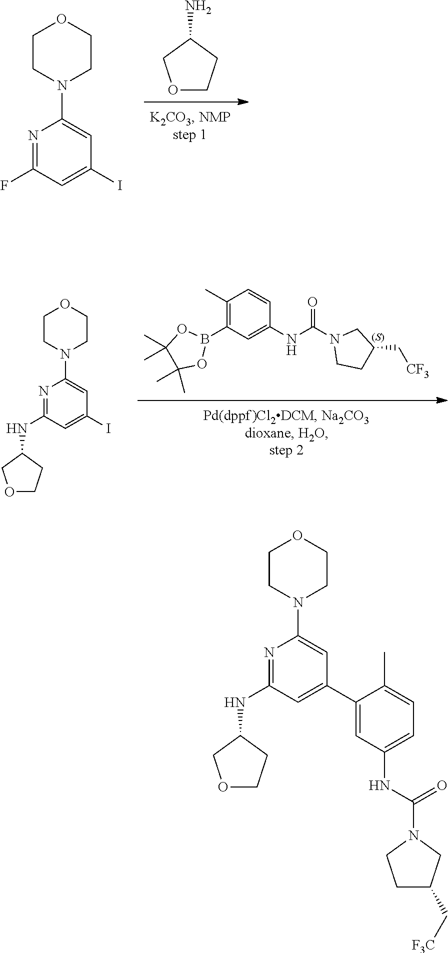

Preparation 43A: 4-iodo-6-(morpholin-4-yl)-N-[(3R)-oxolan-3-yl]pyridin-2-amine

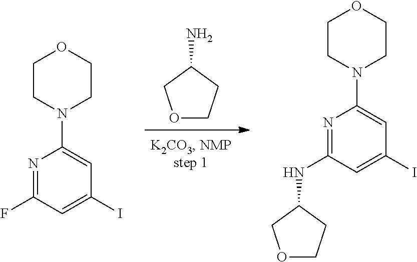 [SMILES: Fc1cc(I)cc(N2CCOCC2)n1 ; N[C@@H]1CCOC1 ; Ic1cc(N[C@@H]2CCOC2)nc(N2CCOCC2)c1]

To a stirred mixture of 4-(6-fluoro-4-iodopyridin-2-yl)morpholine (300 mg, 0.974 mmol) and K₂CO₃ (404 mg, 2.921 mmol) in NMP (3 mL) was added (3R)-oxolan-3-amine (127 mg, 1.461 mmol) at room temperature. The resulting mixture was stirred for 16 h at 150° C. The mixture was allowed to cool down to room temperature. The residue was purified by reverse flash chromatography with the following conditions: column, C18 silica gel; mobile phase, CH₃CN in water, 30% to 70% gradient in 20 min; detector, UV 254 nm to afford 4-iodo-6-(morpholin-4-yl)-N-[(3R)-oxolan-3-yl]pyridin-2-amine (230 mg, 63%) as a brown solid. MS ESI calculated for C₁₃H₁₈IN₃O₂[M+H]⁺ 376.04 found 375.90. ¹H NMR (300 MHz, chloroform-d) δ 6.32 (d, J=1.0 Hz, 1H), 6.20 (d, J=1.0 Hz, 1H), 4.47-4.28 (m, 2H), 4.04-3.91 (m, 2H), 3.91-3.81 (m, 1H), 3.85-3.75 (m, 4H), 3.68 (dd, J=9.2, 3.3 Hz, 1H), 3.56-3.40 (m, 4H), 2.38-2.20 (m, 1H), 1.92-1.79 (m, 1H).

### Example 43: (3S)-N-[4-methyl-3-[2-(morpholin-4-yl)-6-[(3R)-oxolan-3-ylamino]pyridin-4-yl]phenyl]-3-(2,2,2-trifluoroethyl)pyrrolidine-1-carboxamide

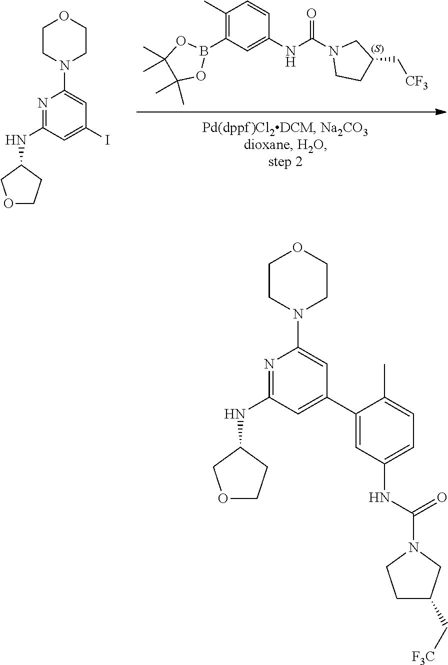

To a stirred mixture of 4-iodo-6-(morpholin-4-yl)-N-[(3R)-oxolan-3-yl]pyridin-2-amine (130 mg, 0.346 mmol,) and (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (142.84 mg, 0.346 mmol,) in 1,4-dioxane (4 mL) and H₂O (1 mL) were added Na₂CO₃ (110 mg, 1.039 mmol) and Pd(dppf)Cl₂·CH₂Cl₂ (29 mg, 0.035 mmol) at 70° C. under nitrogen atmosphere. The reaction was quenched by the addition of water (10 mL) at room temperature. The resulting mixture was extracted with EtOAc (3×40 mL). The combined organic layers were washed with brine (2×4 mL), dried over anhydrous Na₂SO₄. After filtration, the filtrate was concentrated under reduced pressure. The residue was purified by silica gel column chromatography, eluted with PE/EtOAc (1:1) to afford (3S)-N-[4-methyl-3-[2-(morpholin-4-yl)-6-[(3R)-oxolan-3-ylamino]pyridin-4-yl]phenyl]-3-(2,2,2-trifluoroethyl)pyrrolidine-1-carboxamide (55 mg, 30%) as a light green solid. MS ESI calculated for C₂₇H₃₄F₃N₅O₄ [M+H]⁺ 534.26 found 534.10. ¹H NMR (300 MHz, DMSO-d₆) δ 8.12 (s, 1H), 7.49-7.30 (m, 2H), 7.10 (d, J=8.3 Hz, 1H), 6.53 (s, 1H), 5.79 (d, J=6.6 Hz, 2H), 4.33 (s, 1H), 3.92 (dd, J=8.7, 6.0 Hz, 1H), 3.84 (dd, J=8.3, 7.0 Hz, 1H), 3.77-3.64 (m, 6H), 3.59-3.46 (m, 2H), 3.39 (d, J=4.8 Hz, 4H), 3.29 (dd, J=10.1, 6.7 Hz, 1H), 3.02 (t, J=9.3 Hz, 1H), 2.44 (dd, J=12.8, 7.7 Hz, 3H), 2.36-1.94 (m, 5H), 1.89-1.74 (m, 1H), 1.67 (q, J=9.8 Hz, 1H).

### Example 44: (3S)-N-[4-methyl-3-[2-(morpholin-4-yl)-6-[(3S)-oxolan-3-ylamino]pyridin-4-yl]phenyl]-3-(2,2,2-trifluoroethyl)pyrrolidine-1-carboxamide (FT002912 PH-FNT-KIN-04-1601-0)

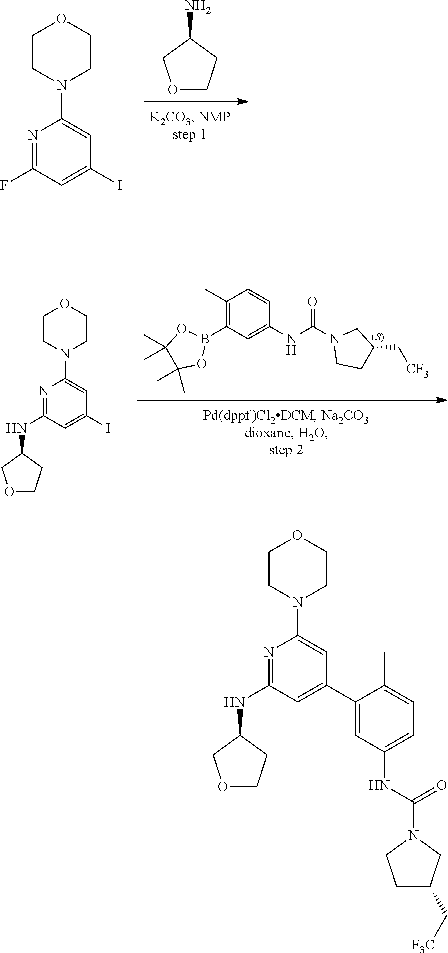

Preparation 44A: 4-iodo-6-(morpholin-4-yl)-N-[(3S)-oxolan-3-yl]pyridin-2-amine

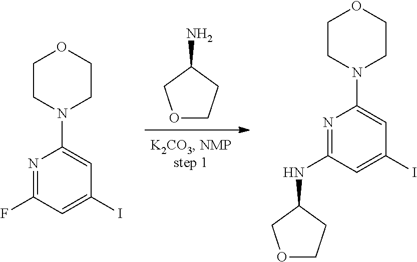 [SMILES: Fc1cc(I)cc(N2CCOCC2)n1 ; N[C@H]1CCOC1 ; Ic1cc(N[C@H]2CCOC2)nc(N2CCOCC2)c1]

To a stirred mixture of 4-(6-fluoro-4-iodopyridin-2-yl)morpholine (300 mg, 0.974 mmol) and K₂CO₃ (404 mg, 2.921 mmol) in NMP (3 mL) was added (3S)-oxolan-3-amine (127 mg, 1.461 mmol) at room temperature. The resulting mixture was stirred for 16 h at 150° C. The reaction was quenched by the addition of water (20 mL) at room temperature. The resulting mixture was extracted with EtOAc (3×50 mL). The combined organic layers were washed with brine (2×5 mL), dried over anhydrous Na₂SO₄. After filtration, the filtrate was concentrated under reduced pressure. The residue was purified by silica gel column chromatography eluted with PE/EtOAc (1/1) to afford 4-iodo-6-(morpholin-4-yl)-N-[(3S)-oxolan-3-yl]pyridin-2-amine (304 mg, 83%) as a light green solid. MS ESI calculated for C₁₃H₁₈IN₃O₂ 376.04, found 375.95. ¹H NMR (400 MHz, chloroform-d) δ 6.32 (d, J=1.1 Hz, 1H), 6.20 (d, J=1.0 Hz, 1H), 4.42 (s, 1H), 3.98 (dd, J=9.0, 5.7 Hz, 2H), 3.86 (td, J=8.4, 5.6 Hz, 1H), 3.80 (t, J=4.9 Hz, 4H), 3.69 (dd, J=9.1, 3.2 Hz, 1H), 3.45 (s, 4H), 2.36-2.22 (m, 1H), 1.60 (s, 1H), 1.28 (s, 1H).

### Example 44: (3S)-N-[4-methyl-3-[2-(morpholin-4-yl)-6-[(3S)-oxolan-3-ylamino]pyridin-4-yl]phenyl]-3-(2,2,2-trifluoroethyl)pyrrolidine-1-carboxamide

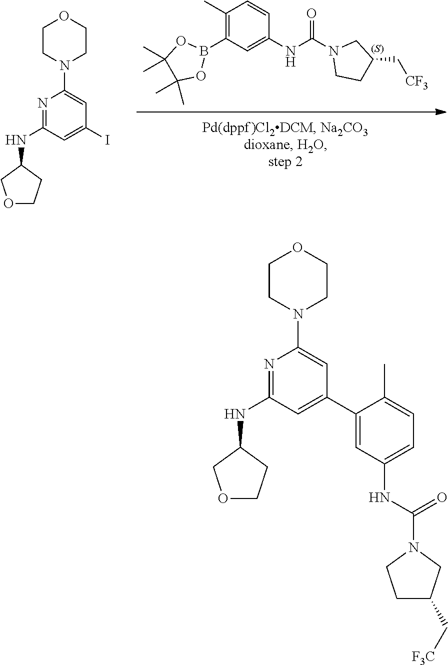

To a stirred mixture of 4-iodo-6-(morpholin-4-yl)-N-[(3S)-oxolan-3-yl]pyridin-2-amine (150 mg, 0.400 mmol) and (3R)-N-[4-methyl-3-(4,4,5,5-tetramethyl-1,3,2-dioxaborolan-2-yl)phenyl]-3-(2,2,2-trifluoroethyl)cyclopentane-1-carboxamide (132 mg, 0.321 mmol) in 1,4-dioxane (4 mL) and H₂O (1 mL) were added Na₂CO₃ (127 mg, 1.199 mmol) and Pd(dppf)Cl₂·CH₂Cl₂ (33 mg, 0.040 mmol) at 150° C. under nitrogen atmosphere. The reaction was quenched by the addition of water (20 mL) at room temperature. The resulting mixture was extracted with EtOAc (3×60 mL). The combined organic layers were washed with brine (2×20 mL), dried over anhydrous Na₂SO₄. After filtration, the filtrate was concentrated under reduced pressure. The residue was purified by Prep-HPLC with the following conditions: Column: SunFire Prep C18 OBD Column, 19×150 mm Sum 10 nm; Mobile Phase A:Water(0.1% FA), Mobile Phase B: CH₃CN; Flow rate:20 mL/min; Gradient:35 B to 70 B in 5.3 min; 254/210 nm to afford (3S)-N-[4-methyl-3-[2-(morpholin-4-yl)-6-[(3S)-oxolan-3-ylamino]pyridin-4-yl]phenyl]-3-(2,2,2-trifluoroethyl)pyrrolidine-1-carboxamide (61 mg, 29%) as a light green solid. MS ESI calculated for 534.26, found 534.15. ¹H NMR (400 MHz, DMSO-d₆) δ 8.11 (s, 1H), 7.51-7.25 (m, 2H), 7.10 (d, J=8.3 Hz, 1H), 6.51 (d, J=5.8 Hz, 1H), 5.88-5.64 (m, 2H), 4.33 (s, 1H), 3.92 (dd, J=8.7, 6.0 Hz, 1H), 3.82 (q, J=7.5 Hz, 1H), 3.68 (t, J=4.9 Hz, 6H), 3.51 (dd, J=8.7, 3.9 Hz, 3H), 3.39 (d, J=5.4 Hz, 5H), 3.02 (t, J=9.4 Hz, 1H), 2.44 (t, J=10.9 Hz, 2H), 2.16 (s, 4H), 2.12-2.04 (m, 1H), 1.80 (dq, J=12.3, 6.0 Hz, 1H), 1.67 (q, J=10.0 Hz, 1H).

### Example 45: (3S)-N-[4-methyl-3-[2-(morpholin-4-yl)-6-[(3R)-oxan-3-ylamino]pyridin-4-yl]phenyl]-3-(2,2,2-trifluoroethyl)pyrrolidine-1-carboxamide

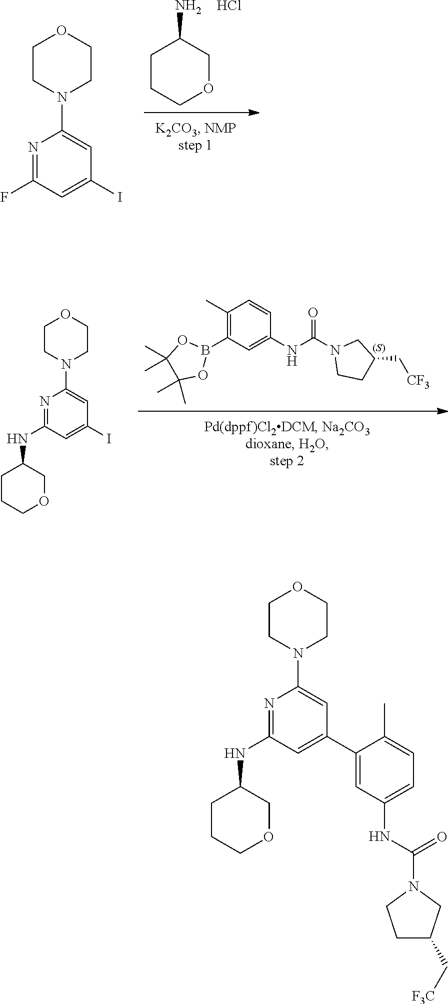

Preparation 45A: 4-iodo-6-(morpholin-4-yl)-N-[(3R)-oxan-3-yl]pyridin-2-amine

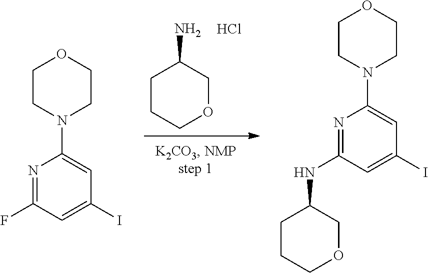 [SMILES: Fc1cc(I)cc(N2CCOCC2)n1 ; N[C@@H]1CCCOC1 ; Cl ; Ic1cc(N[C@@H]2CCCOC2)nc(N2CCOCC2)c1]

To a stirred mixture of 4-(6-fluoro-4-iodopyridin-2-yl)morpholine (300 mg, 0.974 mmol) and K₂CO₃ (673 mg, 4.869 mmol), (3R)-oxan-3-amine hydrochloride (670 mg, 4.869 mmol) in NMP (3 mL) at room temperature under nitrogen atmosphere. The resulting mixture was stirred for 16 h at 150° C. under nitrogen atmosphere. The resulting mixture was diluted with water (50 mL). The resulting mixture was extracted with EtOAc (3×50 mL). The combined organic layers were washed with brine (2×50 mL), dried over anhydrous Na₂SO₄. After filtration, the filtrate was concentrated under reduced pressure. The residue was purified by silica gel column chromatography, eluted with hexane/EtOAc/EtOH (4/3/1) to afford 4-iodo-6-(morpholin-4-yl)-N-[(3R)-oxan-3-yl]pyridin-2-amine (322 mg, 85%) as a white solid. MS ESI calculated for C₁₄H₂₀IN₃O₂[M+H]⁺, 390.06, found 389.95.

### Example 45: (3S)-N-[4-methyl-3-[2-(morpholin-4-yl)-6-[(3R)-oxan-3-ylamino]pyridin-4-yl]phenyl]-3-(2,2,2-trifluoroethyl)pyrrolidine-1-carboxamide

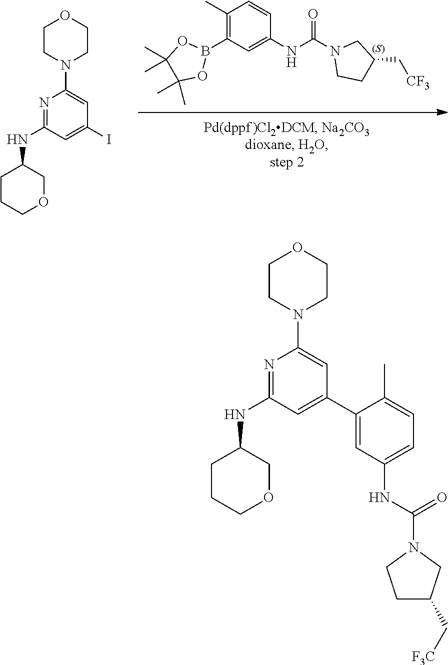

To a stirred mixture of 4-iodo-6-(morpholin-4-yl)-N-[(3R)-oxan-3-yl]pyridin-2-amine (150 mg, 0.385 mmol) and (3R)-N-[4-methyl-3-(4,4,5,5-tetramethyl-1,3,2-dioxaborolan-2-yl)phenyl]-3-(2,2,2-trifluoroethyl)cyclopentane-1-carboxamide (127 mg, 0.308 mmol) in 1,4-dioxane (4 mL) and H₂O (1 mL) were added Na₂CO₃ (122 mg, 1.156 mmol) and Pd(dppf)Cl₂·CH₂Cl₂ (31 mg, 0.039 mmol) was stirred for 2 h at 150° C. under nitrogen atmosphere. The reaction was quenched by the addition of water (20 mL) at room temperature. The resulting mixture was extracted with EtOAc (3×60 mL). The combined organic layers were washed with brine (2×10 mL), dried over anhydrous Na₂SO₄. After filtration, the filtrate was concentrated under reduced pressure. The residue was purified by silica gel column chromatography, eluted with PE/EtOAc (1:1) to afford (3S)-N-[4-methyl-3-[2-(morpholin-4-yl)-6-[(3R)-oxan-3-ylamino]pyridin-4-yl]phenyl]-3-(2,2,2-trifluoroethyl)pyrrolidine-1-carboxamide (25 mg, 12%) as a light green solid. MS ESI calculated for C₂₈H₃₆F₃N₅O₃[M+H]⁺, 547.28 found 548.15. ¹H NMR (400 MHz, DMSO-d₆) δ 8.13 (s, 1H), 7.49-7.29 (m, 2H), 7.10 (d, J=8.3 Hz, 1H), 6.20 (brs, 1H), 5.81 (s, 2H), 3.92 (s, 1H), 3.81 (s, 1H), 3.73-3.62 (m, 6H), 3.52 (ddd, J=10.3, 8.3, 2.3 Hz, 1H), 3.36-3.30 (m, 8H), 2.48-2.32 (m, 3H), 2.16 (s, 3H), 2.11-2.06 (m, 1H), 1.92-1.90 (m, 1H), 1.76-1.59 (m, 4H).

### Example 46: (3S)-N-[4-methyl-3-[2-(morpholin-4-yl)-6-(oxan-4-ylamino)pyridin-4-yl]phenyl]-3-(2,2,2-trifluoroethyl)pyrrolidine-1-carboxamide

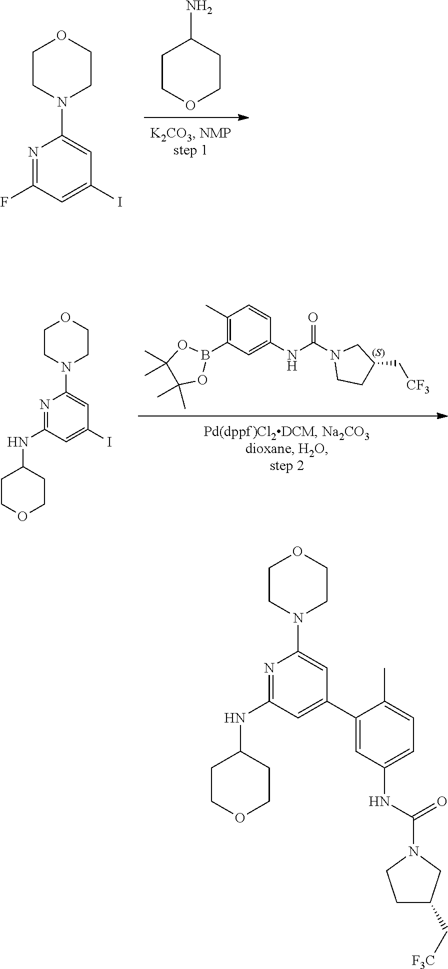

Preparation 46A: 4-iodo-6-(morpholin-4-yl)-N-(oxan-4-yl)pyridin-2-amine

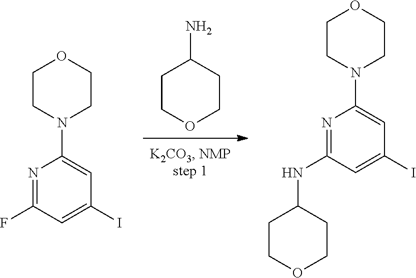 [SMILES: Fc1cc(I)cc(N2CCOCC2)n1 ; NC1CCOCC1 ; Ic1cc(NC2CCOCC2)nc(N2CCOCC2)c1]

To a stirred mixture of 4-(6-fluoro-4-iodopyridin-2-yl)morpholine (300 mg, 0.974 mmol) and oxan-4-amine (541.70 mg, 5.355 mmol) in NMP (3 mL) was added K₂CO₃ (269.15 mg, 1.947 mmol) at 60° C. under nitrogen atmosphere. The reaction was quenched by the addition of water (10 mL) at room temperature. The resulting mixture was extracted with EtOAc (3×50 mL). The combined organic layers were washed with brine (2×5 mL), dried over anhydrous Na₂SO₄. After filtration, the filtrate was concentrated under reduced pressure. The residue was purified by silica gel column chromatography, eluted with PE/EtOAc (1:1) to afford 4-iodo-6-(morpholin-4-yl)-N-(oxan-4-yl)pyridin-2-amine (150 mg, 40%) as a light green solid. MS ESI calculated for C₁₄H₂₀IN₃O₂[M+H]⁺, 390.06, found 389.95 ¹H NMR (400 MHz, DMSO-d₆) δ 6.38 (d, J=7.3 Hz, 1H), 6.23 (d, J=1.0 Hz, 1H), 6.20 (d, J=1.0 Hz, 1H), 3.84 (dt, J=11.7, 3.7 Hz, 2H), 3.68-3.61 (m, 4H), 3.44-3.30 (m, 4H), 1.84 (d, J=12.2 Hz, 2H), 1.49-1.30 (m, 2H), 1.24 (s, 1H), 0.89 (dd, J=29.8, 6.5 Hz, 2H).

### Example 46: (3S)-N-[4-methyl-3-[2-(morpholin-4-yl)-6-(oxan-4-ylamino)pyridin-4-yl]phenyl]-3-(2,2,2-trifluoroethyl)pyrrolidine-1-carboxamide

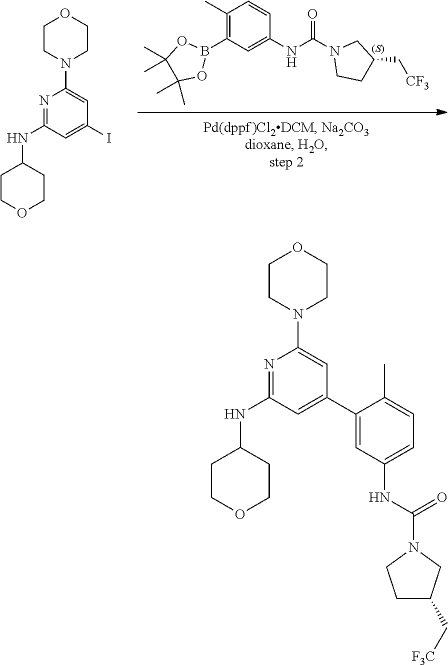

To a stirred mixture of 4-iodo-6-(morpholin-4-yl)-N-(oxan-4-yl)pyridin-2-amine (150 mg, 0.385 mmol) and (3R)-N-[4-methyl-3-(4,4,5,5-tetramethyl-1,3,2-dioxaborolan-2-yl)phenyl]-3-(2,2,2-trifluoroethyl)cyclopentane-1-carboxamide (127 mg, 0.308 mmol) in 1,4-dioxane (4 mL) and H₂O (1 mL) were added Na₂CO₃ (122 mg, 1.156 mmol) and Pd(dppf)Cl₂·CH₂Cl₂ (31 mg, 0.039 mmol) at 150° C. under nitrogen atmosphere. The reaction was quenched by the addition of water (20 mL) at room temperature. The resulting mixture was extracted with EtOAc (3×60 mL). The combined organic layers were washed with brine (2×20 mL), dried over anhydrous Na₂SO₄. After filtration, the filtrate was concentrated under reduced pressure. The residue was purified by Prep-HPLC with the following conditions: Column: SunFire Prep C18 OBD Column, 19×150 mm Sum 10 nm; Mobile Phase A:Water(0.1% FA), Mobile Phase B: CH₃CN; Flow rate:20 mL/min; Gradient:30 B to 75 B in 5.5 min; 254/210 nm to afford (3S)-N-[4-methyl-3-[2-(morpholin-4-yl)-6-(oxan-4-ylamino)pyridin-4-yl]phenyl]-3-(2,2,2-trifluoroethyl)pyrrolidine-1-carboxamide (89 mg, 42%) as a light green solid. MS ESI calculated for C₂₈H₃₆F₃N₅O₃[M+H]⁺, 548.28, found 548.15. ¹H NMR (400 MHz, DMSO-d₆) δ 8.13 (d, J=14.5 Hz, 1H), 7.41 (dd, J=8.3, 2.4 Hz, 1H), 7.34 (d, J=2.3 Hz, 1H), 7.09 (d, J=8.4 Hz, 1H), 6.21 (d, J=7.4 Hz, 1H), 5.76 (s, 2H), 3.95-3.76 (m, 4H), 3.76-3.59 (m, 6H), 3.52 (t, J=8.8 Hz, 1H), 3.45-3.39 (m, 5H), 3.02 (t, J=9.5 Hz, 1H), 2.44 (t, J=11.0 Hz, 3H), 2.16 (s, 3H), 2.08 (s, 1H), 1.90 (d, J=13.2 Hz, 2H), 1.66 (p, J=10.1 Hz, 1H), 1.52-1.32 (m, 2H).

### Example 47: (S)-N-(3-(2-(((1S,3R)-3-hydroxycyclobutyl)amino)-6-morpholinopyridin-4-yl)-4-methylphenyl)-3-(2,2,2-trifluoroethyl)pyrrolidine-1-carboxamide

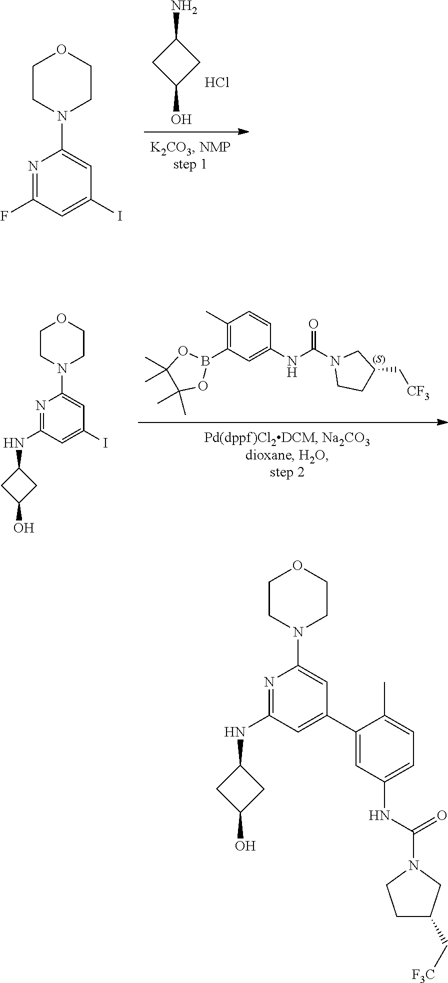

Preparation 47A: (1S,3S)-3-((4-iodo-6-morpholinopyridin-2-yl)amino)cyclobutan-1-ol

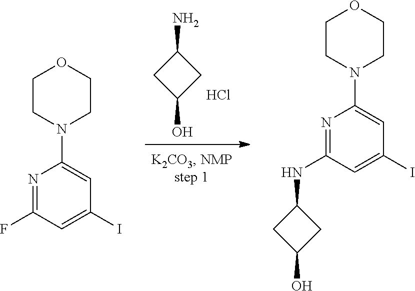 [SMILES: Fc1cc(I)cc(N2CCOCC2)n1 ; N[C@H]1C[C@@H](O)C1 ; Cl ; O[C@H]1C[C@@H](Nc2cc(I)cc(N3CCOCC3)n2)C1]

A mixture of 4-(6-fluoro-4-iodopyridin-2-yl)morpholine (300 mg, 0.974 mmol), (cis)-3-aminocyclobutan-1-ol hydrochloride (181 mg, 1.461 mmol) and K₂CO₃ (404 mg, 2.921 mmol) in NMP (5 mL) was stirred for 16 h at 150° C. under nitrogen atmosphere. The mixture was allowed to cool down to room temperature. The reaction was quenched by the addition of water (50 mL) at room temperature. The resulting mixture was extracted with EtOAc (3×30 mL). The combined organic layers were washed with brine (50 mL), dried over anhydrous Na₂SO₄. After filtration, the filtrate was concentrated under reduced pressure The residue was purified by silica gel column chromatography, eluted with EtOAc in PE (80%) to afford (1S,3S)-3-((4-iodo-6-morpholinopyridin-2-yl)amino)cyclobutan-1-ol (300 mg, 80%) as an off-white solid. MS ESI calculated for C₁₃H₁₈IN₃O₂[M+H]⁺, 376.04, found 375.95. ¹H NMR (400 MHz, chloroform-d) δ 6.31 (s, 1H), 6.12 (s, 1H), 4.45-4.44 (m, 1H), 4.17-4.10 (m, 1H), 3.80-3.78 (m, 4H), 3.68-3.62 (m, 1H), 3.46-3.43 (m, 4H), 2.92-2.85 (m, 1H), 1.83-1.81 (m, 4H).

### Example 47: (S)-N-(3-(2-(((1S,3R)-3-hydroxycyclobutyl)amino)-6-morpholinopyridin-4-yl)-4-methylphenyl)-3-(2,2,2-trifluoroethyl)pyrrolidine-1-carboxamide

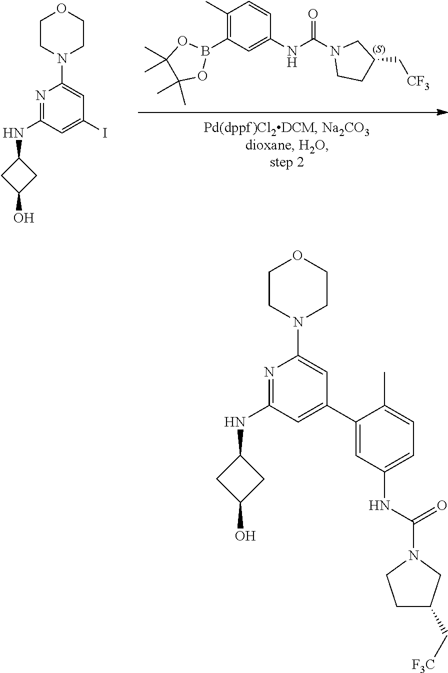

A mixture of (1S,3S)-3-((4-iodo-6-morpholinopyridin-2-yl)amino)cyclobutan-1-ol (200 mg, 0.533 mmol), (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (242 mg, 0.586 mmol), Pd(dppf)Cl₂·CH₂Cl₂ (43 mg, 0.053 mmol), Na₂CO₃ (169 mg, 1.599 mmol), 1,4-dioxane (6 mL) and water (1.5 mL) was stirred for 16 h at 60° C. under nitrogen atmosphere. The reaction mixture was cooled to room temperature and concentrated under reduced pressure. The residue was purified by silica gel column chromatography, eluted with EA/EtOH (3/1) in PE (65%) followed by trituration with DCM/Hexane (1/5) to afford (S)-N-(3-(2-(((1S,3R)-3-hydroxycyclobutyl)amino)-6-morpholinopyridin-4-yl)-4-methylphenyl)-3-(2,2,2-trifluoroethyl)pyrrolidine-1-carboxamide (214 mg, 74%) as a light yellow solid. MS ESI calculated for C₂₇H₃₄F₃N₅O₃ [M+H]⁺, 534.26, found 534.20. ¹H NMR (400 MHz, DMSO-d₆) δ 8.12 (s, 1H), 7.42-7.40 (m, 1H), 7.40 (s, 1H), 7.33-7.32 (m, 1H), 7.10-7.08 (m, 1H), 5.76 (s, 1H), 5.68 (s, 1H), 5.00 (s, 1H), 3.86-3.84 (m, 1H), 3.69-3.65 (m, 6H), 3.52-3.50 (m, 1H), 3.38-3.29 (m, 5H), 3.04-2.99 (m, 1H), 2.63-2.41 (m, 5H), 2.15-2.07 (m, 4H), 1.74-1.66 (m, 3H). ¹⁹F NMR (376 MHz, DMSO-d₆) δ-63.37 (3F).

### Example 48: (S)-N-(3-(2-(((1R,3S)-3-hydroxycyclobutyl)amino)-6-morpholinopyridin-4-yl)-4-methylphenyl)-3-(2,2,2-trifluoroethyl)pyrrolidine-1-carboxamide

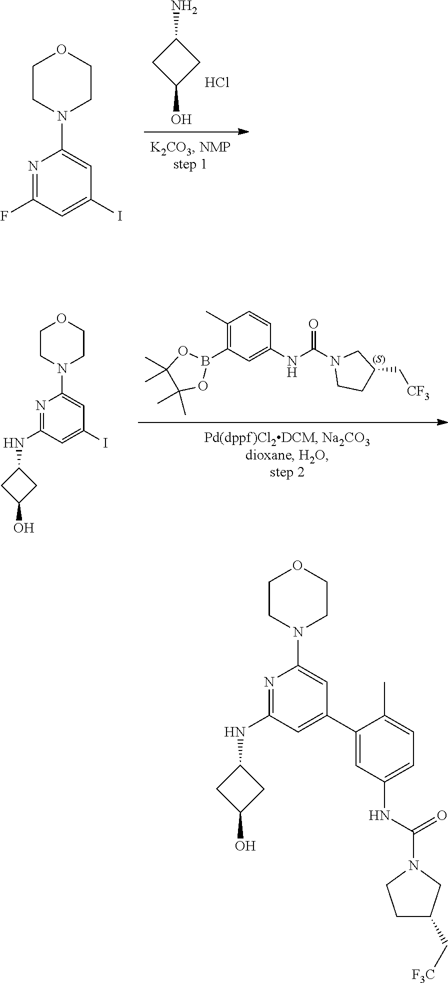

Preparation 48A: (1R,3R)-3-((4-iodo-6-morpholinopyridin-2-yl)amino)cyclobutan-1-ol

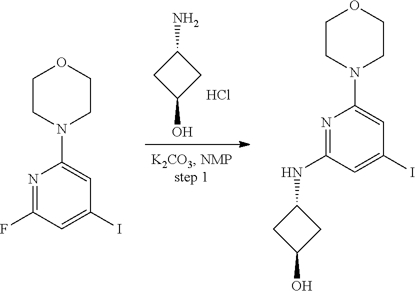 [SMILES: Fc1cc(I)cc(N2CCOCC2)n1 ; N[C@H]1C[C@H](O)C1 ; Cl ; O[C@H]1C[C@H](Nc2cc(I)cc(N3CCOCC3)n2)C1]

A mixture of 4-(6-fluoro-4-iodopyridin-2-yl)morpholine (300 mg, 0.974 mmol), (1R,3R)-3-aminocyclobutan-1-ol hydrochloride (181 mg, 1.461 mmol) and K₂CO₃ (404 mg, 2.921 mmol) in NMP (5 mL) was stirred for 16 h at 150° C. under nitrogen atmosphere. The mixture was allowed to cool down to room temperature. The reaction was quenched by the addition of water (50 mL) at room temperature. The resulting mixture was extracted with EtOAc (3×30 mL). The combined organic layers were washed with brine (50 mL), dried over anhydrous Na₂SO₄. After filtration, the filtrate was concentrated under reduced pressure The residue was purified by silica gel column chromatography, eluted with EtOAc in PE (80%) to afford (1R,3R)-3-((4-iodo-6-morpholinopyridin-2-yl)amino)cyclobutan-1-ol (270 mg, 74%) as a light yellow solid. MS ESI calculated for C₁₃H₁₈IN₃O₂ [M+H]⁺, 376.04, found 375.90. ¹H NMR (400 MHz, DMSO-d₆) δ 6.63-6.60 (m, 1H), 6.22 (s, 1H), 6.11 (s, 1H), 4.96 (s, 1H), 4.29-4.13 (m, 2H), 3.65-3.63 (m, 4H), 3.41-3.33 (m, 4H), 2.16-2.06 (m, 4H).

### Example 48: (S)-N-(3-(2-(((1R,3S)-3-hydroxycyclobutyl)amino)-6-morpholinopyridin-4-yl)-4-methylphenyl)-3-(2,2,2-trifluoroethyl)pyrrolidine-1-carboxamide

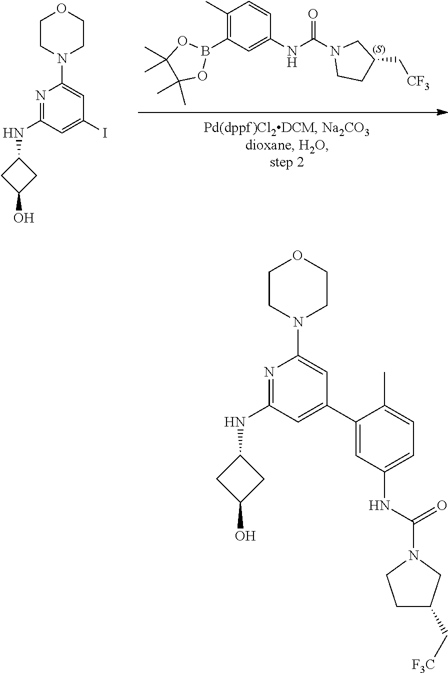

A mixture of (1R,3R)-3-((4-iodo-6-morpholinopyridin-2-yl)amino)cyclobutan-1-ol (200 mg, 0.533 mmol), (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (242 mg, 0.586 mmol), Pd(dppf)Cl₂·CH₂Cl₂ (43 mg, 0.053 mmol), Na₂CO₃ (169 mg, 1.599 mmol), 1,4-dioxane (6 mL) and water (1.5 mL). The resulting mixture was stirred for 16 h at 60° C. under nitrogen atmosphere. The reaction mixture was cooled to room temperature and concentrated under reduced pressure. The residue was purified by silica gel column chromatography, eluted with EA/EtOH (3/1) in PE (65%) followed by trituration with DCM/Hexane (1/5) to afford (S)-N-(3-(2-(((1R,3S)-3-hydroxycyclobutyl)amino)-6-morpholinopyridin-4-yl)-4-methylphenyl)-3-(2,2,2-trifluoroethyl)pyrrolidine-1-carboxamide (190 mg, 65%) as a brown yellow solid. MS ESI calculated for C₂₇H₃₄F₃N₅O₃[M+H]⁺, 534.26, found 534.30. ¹H NMR (400 MHz, DMSO-d₆) δ 8.12 (s, 1H), 7.42-7.41 (m, 1H), 7.40 (s, 1H), 7.33-7.32 (m, 1H), 7.10-7.08 (m, 1H), 5.78 (s, 1H), 5.63 (s, 1H), 4.97-4.95 (m, 1H), 4.31-4.18 (m, 2H), 3.69-3.50 (m, 6H), 3.39-3.29 (m, 5H), 3.04-2.99 (m, 1H), 2.50-2.41 (m, 3H), 2.16-2.08 (m, 8H), 1.68-1.66 (m, 1H). ¹⁹F NMR (376 MHz, DMSO-d₆) δ-63.37 (3F).

### Example 49: (3S)-N-[3-(2-[[(2R)-1-hydroxybutan-2-yl]amino]-6-(morpholin-4-yl)pyridin-4-yl)-4-methylphenyl]-3-(2,2,2-trifluoroethyl)pyrrolidine-1-carboxamide

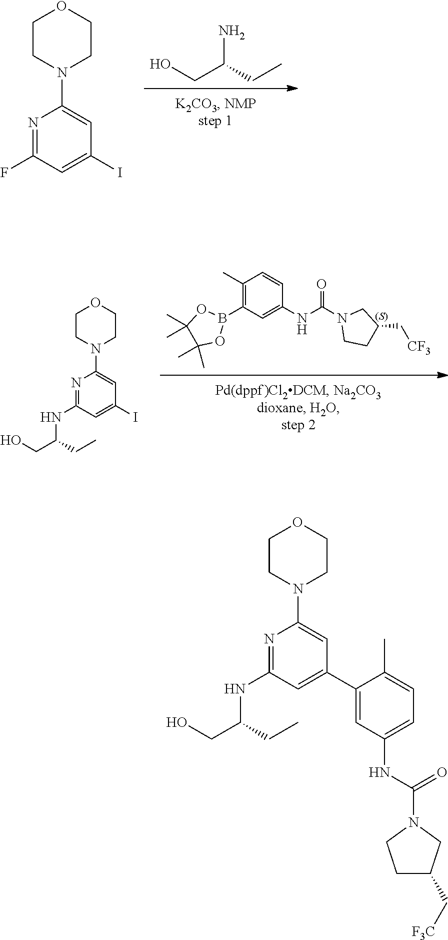

Preparation 49A: (2R)-2-[[4-iodo-6-(morpholin-4-yl)pyridin-2-yl]amino]butan-1-ol

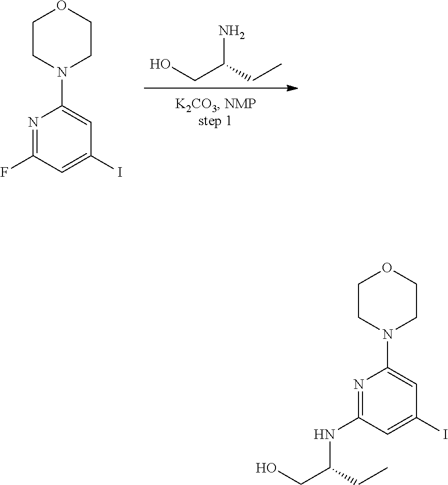

A mixture of 4-(6-fluoro-4-iodopyridin-2-yl)morpholine (300 mg, 0.974 mmol) and K₂CO₃ (269 mg, 1.947 mmol), NMP (3 mL) and (2R)-2-aminobutan-1-ol (130 mg, 1.461 mmol) was stirred for 16 h at 150° C. under nitrogen atmosphere. The reaction was quenched by the addition of water (100 mL). The resulting mixture was extracted with EtOAc (3×50 mL). The combined organic layers were washed with saturated brine (3×50 mL), dried over anhydrous Na₂SO₄. After filtration, the filtrate was concentrated under reduced pressure. The residue was purified by silica gel column chromatography, eluted with PE/EtOAc (2/1) to afford (2R)-2-[[4-iodo-6-(morpholin-4-yl)pyridin-2-yl]amino]butan-1-ol (90 mg, 24%) as an off-white solid. MS ESI calculated for C₁₃H₂₀IN₃O₂[M+H]⁺, 378.06, found 377.90. ¹H NMR (300 MHz, CDCL₃) δ 6.29-6.26 (m, 2H), 4.23 (s, 1H), 3.81-3.76 (m, 6H), 3.62-3.55 (m, 1H), 3.45-3.42 (m, 4H), 3.14 (s, 1H), 1.77-1.63 (m, 1H), 1.57-1.43 (m, 1H), 1.03-0.96 (m, 3H).

### Example 49: (3S)-N-[3-(2-[[(2R)-1-hydroxybutan-2-yl]amino]-6-(morpholin-4-yl)pyridin-4-yl)-4-methylphenyl]-3-(2,2,2-trifluoroethyl)pyrrolidine-1-carboxamide

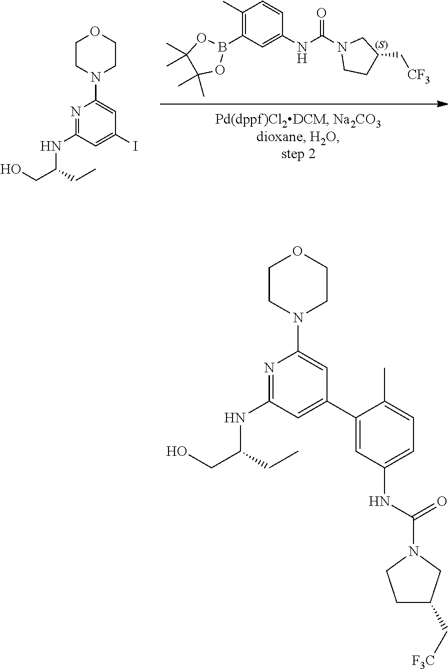

A mixture of (2R)-2-[[4-iodo-6-(morpholin-4-yl)pyridin-2-yl]amino]butan-1-ol (90 mg, 0.239 mmol), (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (98 mg, 0.239 mmol), Na₂CO₃ (76 mg, 0.716 mmol), dioxane:H₂O=4:1 (1 mL) and Pd(dppf)Cl₂·CH₂Cl₂ (19 mg, 0.024 mmol) was stirred for 16 h at 60° C. under nitrogen atmosphere. The reaction was quenched by the addition of water (100 mL).

The resulting mixture was extracted with EtOAc (3×50 mL). The combined organic layers were washed with saturated brine (3×50 mL), dried over anhydrous Na₂SO₄. After filtration, the filtrate was concentrated under reduced pressure. The residue was purified by silica gel column chromatography, eluted with PE/EA/EtOH (8/3/1) to afford 70 mg crude product. The residue was purified by reverse flash chromatography with the following conditions: Column: SunFire Prep C18 OBD Column, 19×150 mm 5 um 10 nm; Mobile Phase A: water (0.1% FA), Mobile Phase B: CH₃CN; Flow rate:20 mL/min; Gradient:25 B to 65 B in 5.1 min; 254/210 nm. This resulted in (3S)-N-[3-(2-[[(2R)-1-hydroxybutan-2-yl]amino]-6-(morpholin-4-yl)pyridin-4-yl)-4-methylphenyl]-3-(2,2,2-trifluoroethyl)pyrrolidine-1-carboxamide (59 mg, 46%) as an off-white solid. MS ESI calculated for C₂₇H₃₆F₃N₅O₃ [M+H]⁺. 536.28, found 536.15. ¹H NMR (400 MHz, DMSO-d₆) δ 8.11 (s, 1H), 7.42-7.40 (m, 1H), 7.34-7.33 (m, 1H), 7.10-7.08 (m, 1H), 5.95-5.93 (m, 1H), 5.78-5.73 (m, 2H), 4.59 (s, 1H), 3.75-3.64 (m, 6H), 3.55-3.48 (m, 2H), 3.38-3.36 (m, 4H), 3.31-3.27 (m, 2H), 3.04-2.99 (m, 1H), 2.48-2.38 (m, 3H), 2.16 (s, 3H), 2.12-2.07 (m, 1H), 1.70-1.60 (m, 2H), 1.48-1.37 (m, 1H), 0.91-0.88 (m, 3H). ¹⁹F NMR (376 MHz, DMSO-d₆) δ-63.37 (3F).

### Example 50: (3S)-N-[3-(2-[[(2R)-1-methoxypropan-2-yl]amino]-6-(morpholin-4-yl)pyridin-4-yl)-4-methylphenyl]-3-(2,2,2-trifluoroethyl)pyrrolidine-1-carboxamide

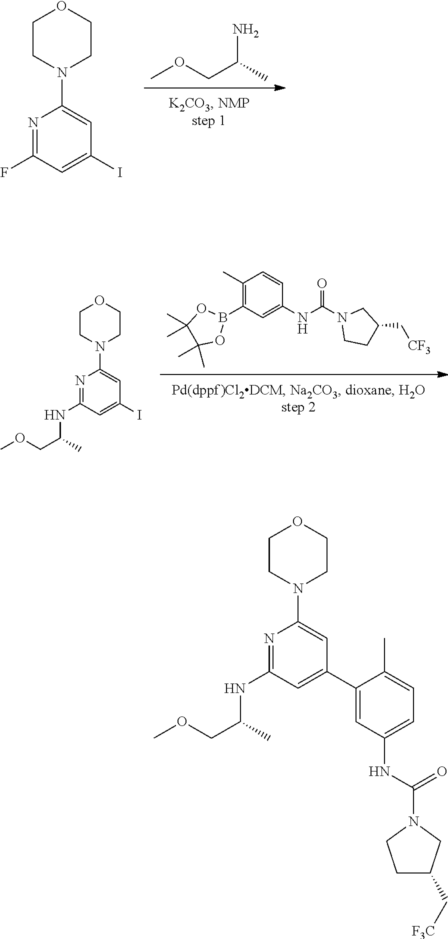

Preparation 50A: 4-iodo-N-[(2R)-1-methoxypropan-2-yl]-6-(morpholin-4-yl)pyridin-2-amine

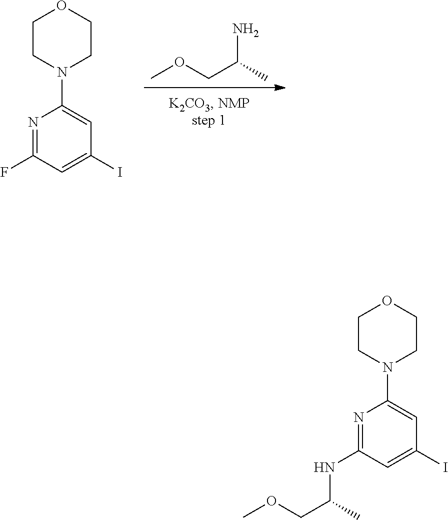

A mixture of 4-(6-fluoro-4-iodopyridin-2-yl)morpholine (300 mg, 0.974 mmol) and K₂CO₃ (269 mg, 1.947 mmol), NMP (3 mL) and (2R)-1-methoxypropan-2-amine (434 mg, 4.869 mmol) was stirred for 16 h at 150° C. under nitrogen atmosphere. The reaction was quenched by the addition of water (100 mL). The resulting mixture was extracted with EtOAc (3×50 mL). The combined organic layers were washed with saturated brine (3×50 mL), dried over anhydrous Na₂SO₄. After filtration, the filtrate was concentrated under reduced pressure. The residue was purified by silica gel column chromatography, eluted with PE/EtOAc (2:1) to afford 4-iodo-N-[(2R)-1-methoxypropan-2-yl]-6-(morpholin-4-yl)pyridin-2-amine (150 mg, 41%) as an off-white solid. MS ESI calculated for C₁₃H₂₀IN₃O₂[M+H]⁺, 378.06, found 377.95. ¹H NMR (400 MHz, CDCL₃) δ 6.27 (m, 1H), 6.20 (s, 1H), 4.45-4.43 (m, 1H), 4.00-3.97 (m, 1H), 3.84-3.78 (m, 4H), 3.53-3.27 (m, 8H), 1.28-1.23 (m, 3H).

### Example 50: (3S)-N-[3-(2-[[(2R)-1-methoxypropan-2-yl]amino]-6-(morpholin-4-yl)pyridin-4-yl)-4-methylphenyl]-3-(2,2,2-trifluoroethyl)pyrrolidine-1-carboxamide

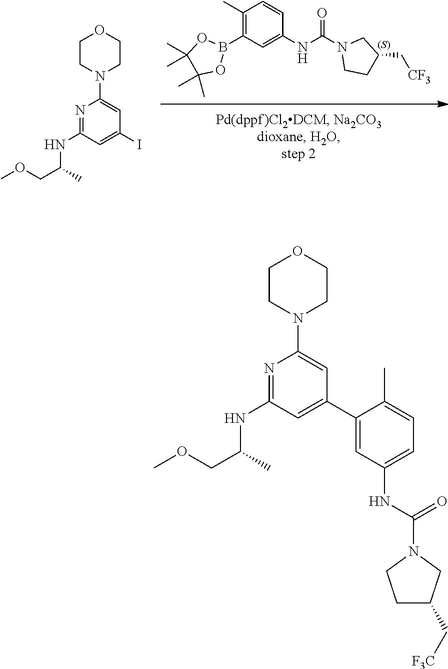

A mixtue of 4-iodo-N-[(2R)-1-methoxypropan-2-yl]-6-(morpholin-4-yl)pyridin-2-amine (150 mg, 0.398 mmol), (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (172 mg, 0.418 mmol), Na₂CO₃ (126 mg, 1.193 mmol), dioxane:H₂O=4:1 (2 mL) was added Pd(dppf)Cl₂·CH₂Cl₂ (32 mg, 0.040 mmol) was stirred for 16 h at 60° C. under nitrogen atmosphere. The reaction was quenched by the addition of water (100 mL). The resulting mixture was extracted with EtOAc (3×50 mL). The combined organic layers were washed with saturated brine (3×50 mL), dried over anhydrous Na₂SO₄. After filtration, the filtrate was concentrated under reduced pressure. The residue was purified by silica gel column chromatography, eluted with PE/EA/EtOH (8/3/1) to afford 70 mg crude product. The residue was purified by reverse flash chromatography with the following conditions: Column: Welch Xtimate C18, 21.2*250 mm, 5 um; Mobile Phase A: water (0.1% FA), Mobile Phase B: CH₃CN; Flow rate:20 mL/min; Gradient:20 B to 60 B in 5.5 min; 254/210 nm; This resulted in (3S)-N-[3-(2-[[(2R)-1-methoxypropan-2-yl]amino]-6-(morpholin-4-yl)pyridin-4-yl)-4-methylphenyl]-3-(2,2,2-trifluoroethyl)pyrrolidine-1-carboxamide (41 mg, 19%) as an off-white solid. MS ESI calculated for C₂₇H₃₆F₃N₅O₃ [M+H]⁺. 536.28, found 536.20. ¹H NMR (400 MHz, DMSO-d₆) δ 8.13 (s, 1H), 7.44-7.36 (m, 2H), 7.12-7.09 (m, 1H), 6.12-6.09 (m, 1H), 5.77 (s, 2H), 4.14-4.10 (m, 1H), 3.77-3.69 (m, 6H), 3.57-3.36 (m, 5H), 3.29 (s, 4H), 3.27-3.22 (m, 1H), 3.07-3.00 (m, 1H), 32.48-2.41 (m, 3H), 2.17 (s, 3H), 2.08-2.03 (m, 1H), 1.71-1.64 (m, 1H), 1.16-1.14 (m, 3H). ¹⁹F NMR (376 MHz, DMSO-d₆) δ-63.36 (3F).

### Example 51: (3S)-N-[3-(2-[[(2S)-2-methoxypropyl]amino]-6-(morpholin-4-yl)pyridin-4-yl)-4-methylphenyl]-3-(2,2,2-trifluoroethyl)pyrrolidine-1-carboxamide

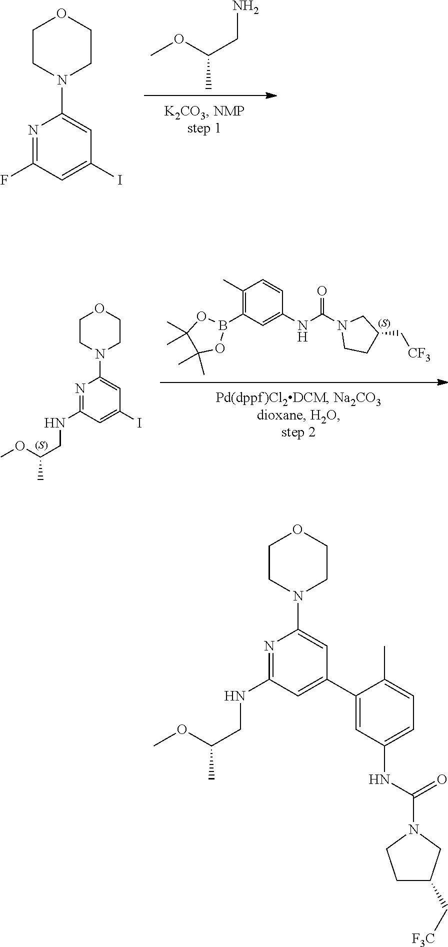

Preparation 51A: 4-iodo-N-[(2S)-2-methoxypropyl]-6-(morpholin-4-yl)pyridin-2-amine

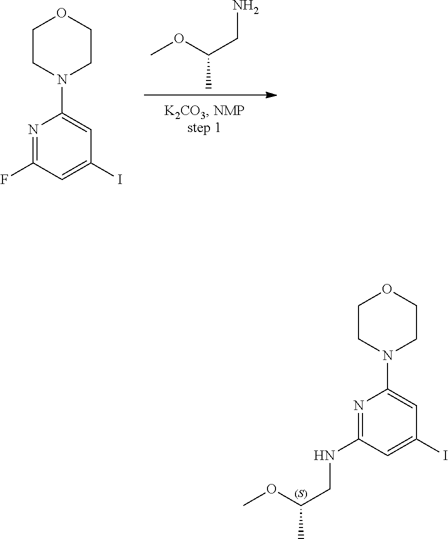

A mixture of 4-(6-fluoro-4-iodopyridin-2-yl) morpholine (300 mg, 0.974 mmol) and (2S)-2-methoxypropan-1-amine (87 mg, 0.974 mmol), NMP (3 mL) and K₂CO₃ (269 mg, 1.947 mmol) was stirred for 16 h at 60° C. under nitrogen atmosphere. The reaction was quenched by the addition of water (15 mL) at room temperature. The resulting mixture was extracted with EtOAc (3×30 mL). The combined organic layers were washed with brine (2×8 mL), dried over anhydrous Na₂SO₄. After filtration, the filtrate was concentrated under reduced pressure. The residue was purified by silica gel column chromatography, eluted with PE/EtOAc (1/1) to afford 4-iodo-N-[(2S)-2-methoxypropyl]-6-(morpholin-4-yl)pyridin-2-amine (120 mg, crude) as a light green soild, which was used for next step without further purification. MS ESI calculated for C₁₃H₂₀IN₃O₂ 378.06, found 378.00.

### Example 51: (3S)-N-[3-(2-[[(2S)-2-methoxypropyl]amino]-6-(morpholin-4-yl)pyridin-4-yl)-4-methylphenyl]-3-(2,2,2-trifluoroethyl)pyrrolidine-1-carboxamide

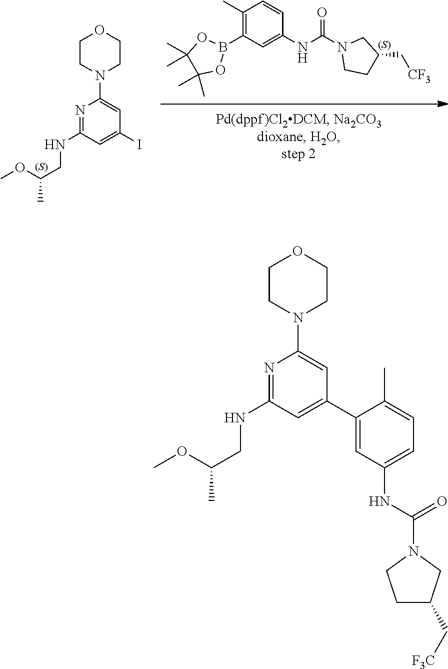

A mixture of 4-iodo-N-[(2S)-2-methoxypropyl]-6-(morpholin-4-yl)pyridin-2-amine (112 mg, 0.297 mmol), (3R)-N-[4-methyl-3-(4,4,5,5-tetramethyl-1,3,2-dioxaborolan-2-yl)phenyl]-3-(2,2,2-trifluoroethyl)cyclopentane-1-carboxamide (98 mg, 0.238 mmol), 1,4-dioxane (4 mL), H₂O (1 mL), Na₂CO₃ (94 mg, 0.891 mmol) and Pd(dppf)Cl₂·CH₂Cl₂ (24 mg, 0.030 mmol) was stirred at 70° C. under nitrogen atmosphere. The reaction was quenched by the addition of water (10 mL) at room temperature. The resulting mixture was extracted with EtOAc (3×30 mL). The combined organic layers were washed with brine (2×8 mL), dried over anhydrous Na₂SO₄. After filtration, the filtrate was concentrated under reduced pressure. The residue was purified by silica gel column chromatography, eluted with PE/EtOAc (1/1) to afford (3S)-N-[3-(2-[[(2S)-2-methoxypropyl]amino]-6-(morpholin-4-yl)pyridin-4-yl)-4-methylphenyl]-3-(2,2,2-trifluoroethyl)pyrrolidine-1-carboxamide (22 mg, %) as a light green solid. MS ESI calculated for C₂₇H₃₆F₃N₅O₃ 536.28, found 536.00. ¹H NMR (400 MHz, DMSO-d₆) δ 8.12 (s, 1H), 7.42 (dd, J=8.3, 2.3 Hz, 1H), 7.33 (d, J=2.4 Hz, 1H), 7.09 (d, J=8.4 Hz, 1H), 6.29 (t, J=5.9 Hz, 1H), 5.78 (d, J=15.3 Hz, 2H), 3.71-3.62 (m, 5H), 3.57-3.45 (m, 2H), 3.38 (t, J=4.9 Hz, 4H), 3.32 (s, 6H), 3.02 (t, J=9.4 Hz, 1H), 2.50 (s, 3H), 2.16 (s, 3H), 2.12-2.05 (m, 1H), 1.64 (q, J=10.2 Hz, 1H), 1.10 (d, J=6.2 Hz, 3H).

### Example 52: (3S)-N-[4-methyl-3-[2-(morpholin-4-yl)-6-[(3S)-oxan-3-ylamino]pyridin-4-yl]phenyl]-3-(2,2,2-trifluoroethyl)pyrrolidine-1-carboxamide

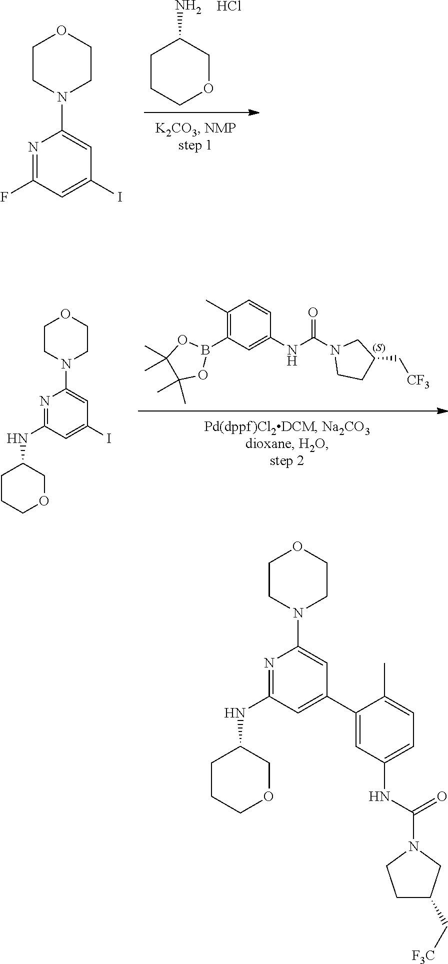

Preparation 52A: 4-iodo-6-(morpholin-4-yl)-N-[(3S)-oxan-3-yl]pyridin-2-amine

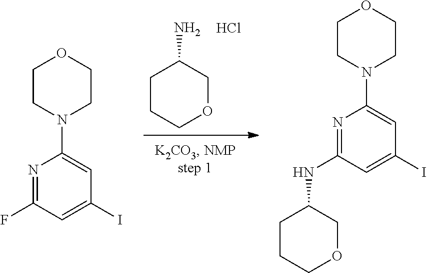 [SMILES: Fc1cc(I)cc(N2CCOCC2)n1 ; N[C@H]1CCCOC1 ; Cl ; Ic1cc(N[C@H]2CCCOC2)nc(N2CCOCC2)c1]

To a stirred mixture of 4-(6-fluoro-4-iodopyridin-2-yl)morpholine (300 mg, 0.974 mmol) and K₂CO₃ (673 mg, 4.869 mmol), (3S)-oxan-3-amine hydrochloride (670 mg, 4.869 mmol) in NMP (3 mL) at room temperature under nitrogen atmosphere. The resulting mixture was stirred for 16 h at 150° C. under nitrogen atmosphere. The resulting mixture was diluted with water (50 mL).

The resulting mixture was extracted with EtOAc (3×50 mL). The combined organic layers were washed with brine (2×50 mL), dried over anhydrous Na₂SO₄. After filtration, the filtrate was concentrated under reduced pressure. The residue was purified by silica gel column chromatography, eluted with hexane/EtOAc/EtOH (4/3/1) to afford 4-iodo-6-(morpholin-4-yl)-N-[(3S)-oxan-3-yl]pyridin-2-amine (260 mg, 69%) as a white solid. MS ESI calculated for C₁₄H₂₀IN₃O₂[M+H]⁺, 390.06, found 389.95. ¹H NMR (300 MHz, chloroform-d) δ 6.33-6.32 (m, 1H), 6.11-6.10 (s, 1H), 4.47-4.45 (m, 1H), 4.23-4.11 (m, 1H), 3.80-3.77 (m, 4H), 3.45-3.42 (m, 4H), 2.60-2.53 (m, 1H), 2.00-1.93 (m, 2H), 1.46 (s, 3H), 1.31-1.26 (m, 1H), 0.91-0.88 (m, 1H).

### Example 52: (3S)-N-[4-methyl-3-[2-(morpholin-4-yl)-6-[(3S)-oxan-3-ylamino]pyridin-4-yl]phenyl]-3-(2,2,2-trifluoroethyl)pyrrolidine-1-carboxamide

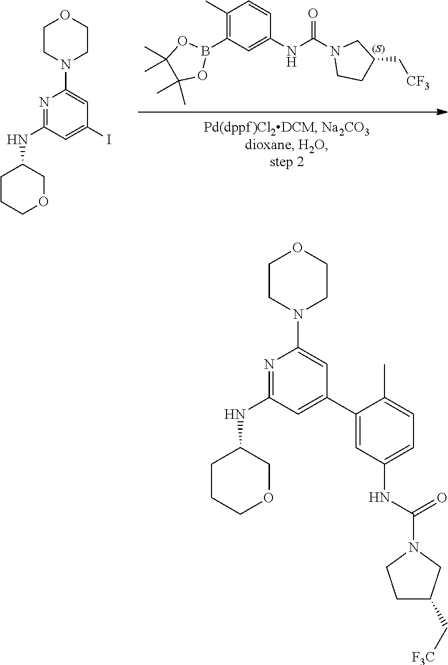

A mixture of 4-iodo-6-(morpholin-4-yl)-N-[(3S)-oxan-3-yl]pyridin-2-amine (200 mg, 0.514 mmol), (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (212 mg, 0.514 mmol), dioxane (2 mL), H₂O (0.5 mL), Pd(dppf)Cl₂·CH₂Cl₂ (42 mg, 0.051 mmol) and Na₂CO₃ (163 mg, 1.541 mmol) was stirred for 3 h at 80° C. under nitrogen atmosphere. The mixture was allowed to cool down to rt. The resulting mixture was concentrated under vacuum. The residue was purified by silica gel column chromatography, eluted with PE/EtOAc (0% to 80%) followed by Prep-HPLC with the following conditions Column: SunFire Prep C18 OBD Column, 19?150 mm Sum 10 nm; Mobile Phase A: water (0.1% FA), Mobile Phase B: CH₃CN; Flow rate:20 mL/min; Gradient:25 B to 65 B in 5.3 min; 254/210 nm to afford (3S)-N-[4-methyl-3-[12-(morpholin-4-yl)-6-[(3S)-oxan-3-ylamino]pyridin-4-yl]phenyl]-3-(2,2,2-trifluoroethyl)pyrrolidine-1-carboxamide (120 mg, 43%) as a off-white solid. MS ESI calculated for C₂₈H₃₆F₃N₅O₃ [M+H]⁺, 548.28, found 548.10. ¹H NMR (400 MHz, DMSO-d₆)δ 8.11 (s, 1H), 7.41 (dd, J=8.3, 2.3 Hz, 1H), 7.34 (d, J=2.4 Hz, 1H), 7.09 (d, J=8.3 Hz, 1H), 6.19 (s, 1H), 5.78 (d, J=4.9 Hz, 2H), 3.94 (dd, J=10.8, 4.0 Hz, 1H), 3.82 (dt, J=13.4, 6.3 Hz, 1H), 3.77-3.62 (m, 6H), 3.52 (ddd, J=10.5, 8.3, 2.3 Hz, 1H), 3.41-3.25 (m, 6H), 3.05 (dt, J=24.7, 9.5 Hz, 2H), 2.51-2.34 (m, 3H), 2.16 (s, 3H), 2.08 (dd, J=9.6, 3.5 Hz, 1H), 1.99-1.90 (m, 1H), 1.75-1.50 (in, 4H).

### Example 53: (3S)-N-(4-methyl-3-[2-[(3-methyloxolan-3-yl)amino]-6-(morpholin-4-yl)pyridin-4-yl]phenyl)-3-(2,2,2-trifluoroethyl)pyrrolidine-1-carboxamide

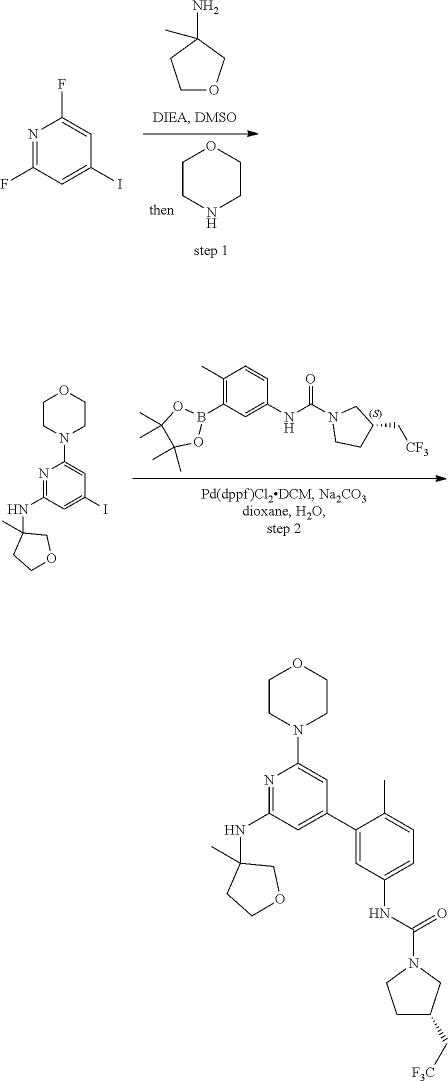

Preparation 53A: 4-iodo-N-(3-methyloxolan-3-yl)-6-(morpholin-4-yl)pyridin-2-amine

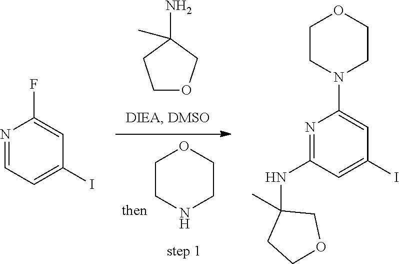

A mixture of 2,6-difluoro-4-iodopyridine (300 mg, 1.245 mmol), 3-methyloxolan-3-amine (138 mg, 1.369 mmol), DMSO (3 mL) and DIEA (0.48 mL, 3.691 mmol) was stirred for 4 h at 110° C. To the above mixture was added morpholine (361 mg, 4.150 mmol) at room temperature. The resulting mixture was stirred for 16 h at 110° C. The mixture was allowed to cool down to room temperature and quenched with water (50 mL). The resulting mixture was extracted with EtOAc (3×10 mL). The combined organic layers were washed with brine (10 mL), dried over anhydrous Na₂SO₄. After filtration, the filtrate was concentrated under reduced pressure. The residue was purified by silica gel column chromatography, eluted with PE/EtOAc (4:1) to afford 4-iodo-N-(3-methyloxolan-3-yl)-6-(morpholin-4-yl)pyridin-2-amine (230 mg, 47%) as a yellow solid. MS ESI calculated for C₁₄H₂₀IN₃O₂[M+H]⁺, 390.05, found 390.05. ¹H NMR (300 MHz, chloroform-d) δ 6.28 (d, J=1.0 Hz, 1H), 6.20 (d, J=1.0 Hz, 1H), 4.39 (s, 1H), 4.00-3.91 (m, 3H), 3.82-3.76 (m, 5H), 3.51-3.37 (m, 4H), 2.41-2.31 (m, 1H), 2.03-1.93 (m, 1H), 1.57 (s, 3H).

### Example 53: (3S)-N-(4-methyl-3-[2-[(3-methyloxolan-3-yl)amino]-6-(morpholin-4-yl)pyridin-4-yl]phenyl)-3-(2,2,2-trifluoroethyl)pyrrolidine-1-carboxamide

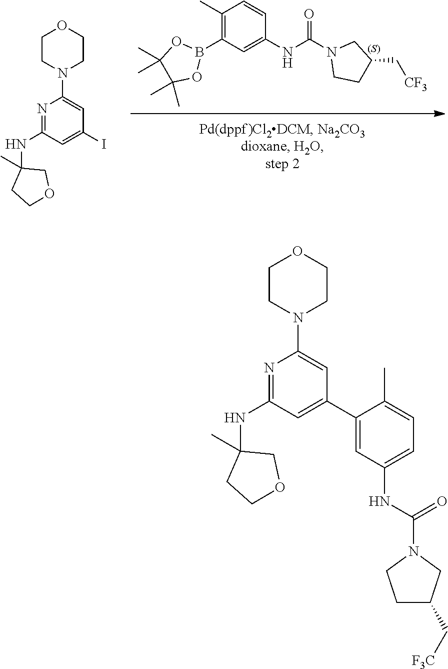

A mixture of 4-iodo-N-(3-methyloxolan-3-yl)-6-(morpholin-4-yl)pyridin-2-amine (230 mg, 0.591 mmol), (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (244 mg, 0.591 mmol), 1,4-dioxane (3 mL), H₂O (0.6 mL), Na₂CO₃ (188 mg, 1.773 mmol) and Pd(dppf)Cl₂·CH₂Cl₂ (48 mg, 0.059 mmol) was stirred for 16 h at 60° C. under nitrogen atmosphere. The mixture was allowed to cool down to room temperature. The reaction was quenched by the addition of water (10 mL) at room temperature.The resulting mixture was extracted with EtOAc (3×10 mL). The combined organic layers were washed with brine (10 mL), dried over anhydrous Na₂SO₄. After filtration, the filtrate was concentrated under reduced pressure. The residue was purified by silica gel column chromatography, eluted with PE/EtOAc (1:2) to afford (3S)-N-(4-methyl-3-[2-[(3-methyloxolan-3-yl)amino]-6-(morpholin-4-yl)pyridin-4-yl]phenyl)-3-(2,2,2-trifluoroethyl)pyrrolidine-1-carboxamide (200 mg, 62%) as a light pink solid. MS ESI calculated for C₂₈H₃₆F₃N₅O₃[M+H]⁺, 548.15, found 548.15. ¹H NMR (400 MHz, DMSO-d₆) δ 8.11 (s, 1H), 7.40 (dd, J=8.3, 2.3 Hz, 1H), 7.35 (d, J=2.3 Hz, 1H), 7.09 (d, J=8.4 Hz, 1H), 6.45 (s, 1H), 5.80 (dd, J=10.3, 1.0 Hz, 2H), 3.95 (d, J=8.5 Hz, 1H), 3.86-3.74 (m, 2H), 3.73-3.67 (m, 6H), 3.57-3.44 (m, 1H), 3.39-3.22 (m, 5H), 3.02 (t, J=9.4 Hz, 1H), 2.49-2.36 (m, 3H), 2.33-2.22 (m, 1H), 2.16 (s, 3H), 2.08 (d, J=11.2 Hz, 1H), 1.95-1.86 (m, 1H), 1.70-1.61 (m, 1H), 1.50 (s, 3H). ¹⁹F NMR (376 MHz, DMSO-d₆) δ-63.61 (3F).

### Example 54: (3S)-N-[3-(2-[[1-(hydroxymethyl)cyclobutyl]amino]-6-(morpholin-4-yl)pyridin-4-yl)-4-methylphenyl]-3-(2,2,2-trifluoroethyl)pyrrolidine-1-carboxamide

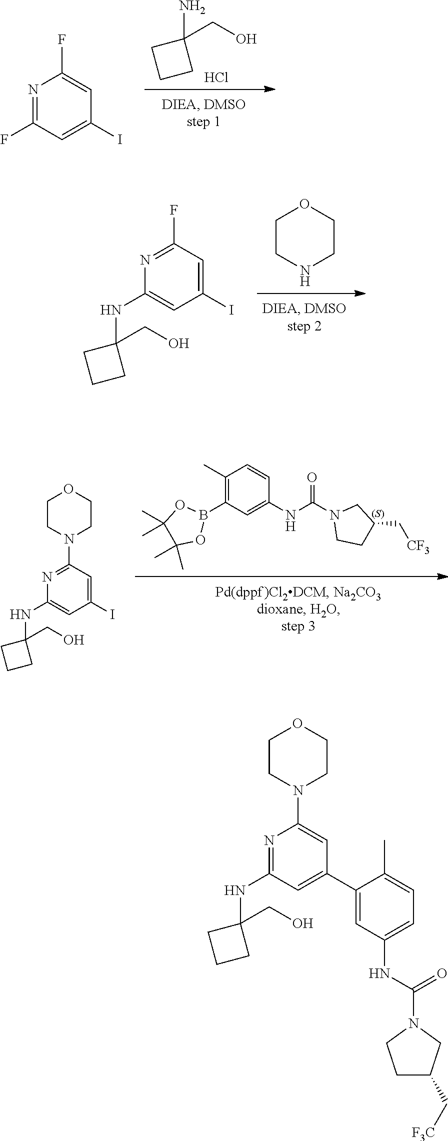

Preparation 54A: [1-[(6-fluoro-4-iodopyridin-2-yl)amino]cyclobutyl]methanol

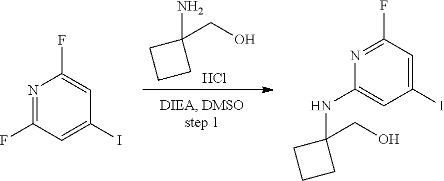 [SMILES: Fc1cc(I)cc(F)n1 ; NC1(CO)CCC1 ; Cl ; OCC1(Nc2cc(I)cc(F)n2)CCC1]

To a mixture of 2,6-difluoro-4-iodopyridine (550 mg, 2.282 mmol), (1-aminocyclobutyl)methanol hydrochloride (345 mg, 2.511 mmol) and DMSO (6 mL) was added DIEA (944 mg, 7.304 mmol) at 0° C. under nitrogen atmosphere. The resulting mixture was stirred for 3 h at 70° C. under nitrogen atmosphere. The reaction was quenched by the addition of water (100 mL). The resulting mixture was extracted with EtOAc (3×50 mL). The combined organic layers were washed with saturated brine (3×50 mL), dried over anhydrous Na₂SO₄. After filtration, the filtrate was concentrated under reduced pressure. The residue was purified by silica gel column chromatography, eluted with PE/EtOAc (2/1) to afford [1-[(6-fluoro-4-iodopyridin-2-yl)amino]cyclobutyl]methanol (320 mg, 43%) as an off-white solid. MS ESI calculated for C₁₀H₁₂FIN₂O [M+H]⁺, 323.00, found 322.85. ¹H NMR (300 MHz, chloroform-d) δ 6.60-6.55 (m, 2H), 3.86 (s, 2H), 2.35-2.25 (m, 2H), 2.17-2.07 (m, 2H), 2.06-1.83 (m, 2H).

Preparation 54B: (1-[[4-iodo-6-(morpholin-4-yl)pyridin-2-yl]amino]cyclobutyl)methanol

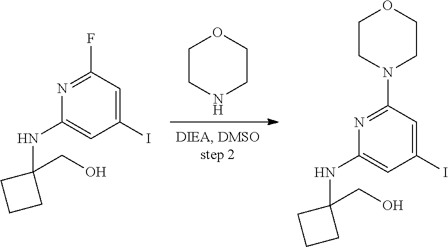 [SMILES: OCC1(Nc2cc(I)cc(F)n2)CCC1 ; C1COCCN1 ; OCC1(Nc2cc(I)cc(N3CCOCC3)n2)CCC1]

To a stirred solution of [1-[(6-fluoro-4-iodopyridin-2-yl)amino]cyclobutyl]methanol (210 mg, 0.652 mmol) and morpholine (125 mg, 1.434 mmol) in DMSO (2.1 mL) was added DIEA (185 mg, 1.434 mmol) at 0° C. under nitrogen atmosphere. The resulting mixture was stirred for 16 h at 110° C. under nitrogen atmosphere. The reaction was quenched by the addition of water (100 mL). The resulting mixture was extracted with EtOAc (3×50 mL). The combined organic layers were washed with saturated brine (3×50 mL), dried over anhydrous Na₂SO₄. After filtration, the filtrate was concentrated under reduced pressure. The residue was purified by silica gel column chromatography, eluted with PE/EtOAc (2/1) to afford (1-[[4-iodo-6-(morpholin-4-yl)pyridin-2-yl]amino]cyclobutyl)methanol (195 mg, 77%) as a light yellow oil. MS ESI calculated for C₁₄H₂₀IN₃O₂[M+H]⁺. 390.06, found 389.95. ¹H NMR (300 MHz, DMSO-d₆) δ 6.45 (s, 1H), 6.24-6.18 (m, 2H), 4.71 (s, 1H), 3.66-3.63 (m, 6H), 3.32-3.29 (m, 4H), 2.13-2.08 (m, 4H), 1.84-1.66 (m, 2H).

### Example 54: (3S)-N-[3-(2-[[1-(hydroxymethyl)cyclobutyl]amino]-6-(morpholin-4-yl)pyridin-4-yl)-4-methylphenyl]-3-(2,2,2-trifluoroethyl)pyrrolidine-1-carboxamide

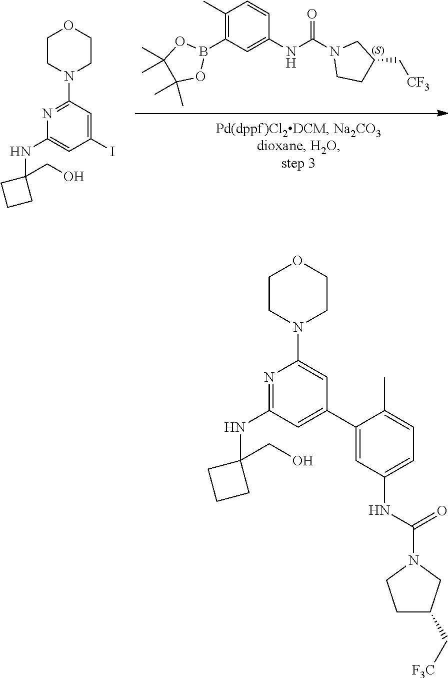

A mixture of (1-[[4-iodo-6-(morpholin-4-yl)pyridin-2-yl]amino]cyclobutyl)methanol (190 mg, 0.488 mmol), (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (201 mg, 0.488 mmol), Na₂CO₃ (155.21 mg, 1.464 mmol), dioxane:H₂O=4:1 (2 mL) and Pd(dppf)Cl₂·CH₂Cl₂ (40 mg, 0.049 mmol) was stirred for 16 h at 60° C. under nitrogen atmosphere. The reaction was quenched by the addition of water (100 mL). The resulting mixture was extracted with EtOAc (3×50 mL). The combined organic layers were washed with saturated brine (3×50 mL), dried over anhydrous Na₂SO₄. After filtration, the filtrate was concentrated under reduced pressure. The residue was purified by silica gel column chromatography, eluted with PE/EA/EtOH (8/3/1) to afford 200 mg crude product followed by reverse flash chromatography with the following conditions: column, C18 silica gel; mobile phase, CH₃CN in water (10 mmol/l NH₄HCO₃), 10% to 80% gradient in 30 min; detector, UV 254 nm. This resulted in (3S)-N-[3-(2-[[1-(hydroxymethyl)cyclobutyl]amino]-6-(morpholin-4-yl)pyridin-4-yl)-4-methylphenyl]-3-(2,2,2-trifluoroethyl)pyrrolidine-1-carboxamide (160 mg, 60%) as an off-white solid. MS ESI calculated for C₂₈H₃₆F₃N₅O₃[M+H]⁺. 548.28, found 548.15.

¹H NMR (400 MHz, DMSO-d₆) δ 8.11 (s, 1H), 7.41-7.35 (m, 2H), 7.11-7.08 (m, 1H), 6.29 (s, 1H), 5.75 (s, 2H), 4.86-4.82 (m, 1H), 3.69-3.67 (m, 8H), 3.56-3.50 (m, 1H), 3.32 (s, 3H), 3.05-2.99 (m, 1H), 2.47-2.40 (m, 3H), 2.16-2.13 (m, 9H), 1.83-1.59 (m, 3H). ¹⁹F NMR (376 MHz, DMSO-d₆) δ-63.36 (3F).

### Example 55: (3S)-N-[3-(2-[[3-(hydroxymethyl)oxetan-3-yl]amino]-6-(morpholin-4-yl)pyridin-4-yl)-4-methylphenyl]-3-(2,2,2-trifluoroethyl)pyrrolidine-1-carboxamide

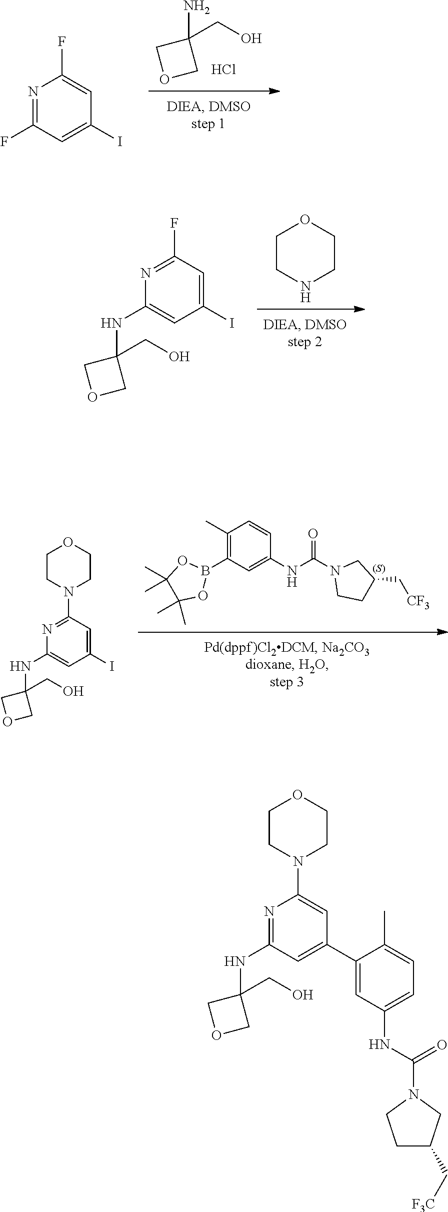

Preparation 55A: [3-[(6-fluoro-4-iodopyridin-2-yl)amino]oxetan-3-yl]methanol

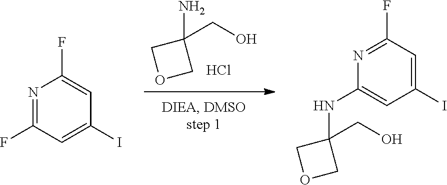 [SMILES: Fc1cc(I)cc(F)n1 ; NC1(CO)COC1 ; Cl ; OCC1(Nc2cc(I)cc(F)n2)COC1]

To a stirred mixture of 2,6-difluoro-4-iodopyridine (1.00 g, 4.150 mmol) and (3-aminooxetan-3-yl)methanol (0.51 g, 4.980 mmol) in DMSO (8 mL) was added DIEA (1.34 g, 10.374 mmol) in portions at room temperature under nitrogen atmosphere. The resulting mixture was stirred for 16 h at 100° C. under nitrogen atmosphere. The resulting mixture was diluted with EtOAc (100 mL). The combined organic layers were washed with brine (2×50 mL), dried over anhydrous Na₂SO₄. After filtration, the filtrate was concentrated under reduced pressure to afford (3-((6-fluoro-4-iodopyridin-2-yl)amino)oxetan-3-yl)methanol (1 g, crude) as light yellow oil. The crude product was used for next step without further purification. MS ESI calculated for C₉H₁₀FIN₂O₂[M+H]⁺, 324.98, found 324.95.

Preparation 55B: (3-[[4-iodo-6-(morpholin-4-yl)pyridin-2-yl]amino]oxetan-3-yl)methanol

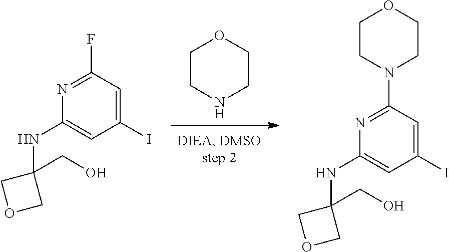 [SMILES: OCC1(Nc2cc(I)cc(F)n2)COC1 ; C1COCCN1 ; OCC1(Nc2cc(I)cc(N3CCOCC3)n2)COC1]

To a stirred mixture of [3-[(6-fluoro-4-iodopyridin-2-yl)amino]oxetan-3-yl]methanol (600 mg, 1.851 mmol, 1.00 equiv) and morpholine (177 mg, 2.036 mmol) in DMSO (4 mL) was added DIEA (526 mg, 4.073 mmol) in portions at room temperature under nitrogen atmosphere. The resulting mixture was stirred for 16 h at 100° C. under nitrogen atmosphere. The resulting mixture was extracted with EtOAc (3×300 mL). The combined organic layers were washed with brine (2×500 mL), dried over anhydrous Na₂SO₄. After filtration, the filtrate was concentrated under reduced pressure. The residue was purified by silica gel column chromatography, eluted with PE/EtOAc (2:1) to afford (3-[[4-iodo-6-(morpholin-4-yl)pyridin-2-yl]amino]oxetan-3-yl)methanol (300 mg, 41.42%) as a white solid. MS ESI calculated for C₁₃H₁₈IN₃O₃[M+H]⁺, 392.04, found 392.04. ¹H NMR (400 MHz, Chloroform-d) δ 7.28 (s, 1H), 6.34 (d, J=1.0 Hz, 1H), 6.28 (s, 1H), 4.73 (d, J=6.4 Hz, 1H), 4.63 (d, J=6.5 Hz, 2H), 4.14 (s, 1H), 3.81-3.77 (m, 2H), 3.37 (s, 2H), 3.13 (s, 1H), 1.59 (s, 2H), 1.34-1.23 (m, 2H), 0.93-0.81 (m, 2H).

### Example 55: (3S)-N-[3-(2-[[3-(hydroxymethyl)oxetan-3-yl]amino]-6-(morpholin-4-yl)pyridin-4-yl)-4-methylphenyl]-3-(2,2,2-trifluoroethyl)pyrrolidine-1-carboxamide

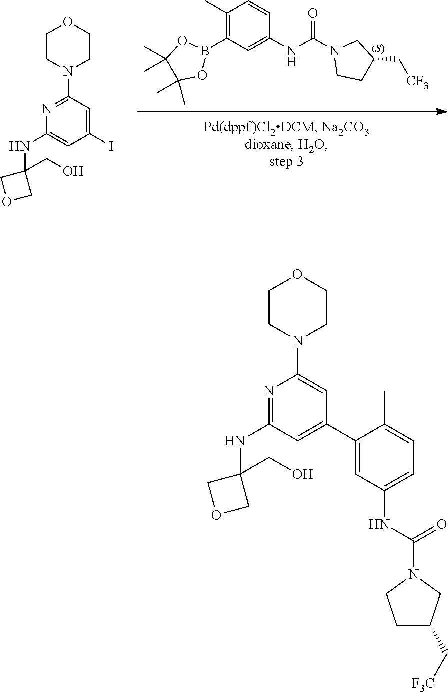

A mixture of (3-[[4-iodo-6-(morpholin-4-yl)pyridin-2-yl]amino]oxetan-3-yl)methanol (250 mg, 0.639 mmol), (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (263 mg, 0.639 mmol), H₂O (0.75 mL) and 1,4-dioxane (3 mL), Pd(dppf)Cl₂·CH₂Cl₂ (52 mg, 0.064 mmol) and Na₂CO₃ (203 mg, 1.917 mmol) was stirred for 2 h at 60° C. under nitrogen atmosphere. The reaction was quenched by the addition of water (100 mL). The resulting mixture was extracted with EtOAc (3×50 mL). The combined organic layers were washed with saturated brine (3×50 mL), dried over anhydrous Na₂SO₄. After filtration, the filtrate was concentrated under reduced pressure. The residue was purified by silica gel column chromatography, eluted with EtOAc followed by reverse phase flash chromatography with the following conditions: Column: Spherical C18, 20-40 μm, 40 g; Mobile Phase A: water (5 mM NH₄HCO₃); Mobile Phase B: CH₃CN; Flow rate: 60 mL/min; Gradient: 5%-5% B, 10 min, 5%-40% B, 35 min, 40% B-98% B gradient in 10 min; Detector: 220 nm to afford (3S)-N-[3-(2-[[3-(hydroxymethyl)oxetan-3-yl]amino]-6-(morpholin-4-yl)pyridin-4-yl)-4-methylphenyl]-3-(2,2,2-trifluoroethyl)pyrrolidine-1-carboxamide (186 mg, 53%) as a light blue solid. MS ESI calculated for C₂₇H₃₄F₃N₅O₄[M+H]⁺, 550.26, found 550.3. ¹H NMR (400 MHz, DMSO-d₆) δ 8.12 (s, 1H), 7.43-7.33 (m, 2H), 7.10 (d, J=8.3 Hz, 1H), 6.81 (s, 1H), 5.87-5.73 (m, 2H), 5.03 (t, J=5.7 Hz, 1H), 4.56 (q, J=6.1 Hz, 4H), 3.81 (d, J=5.8 Hz, 2H), 3.66 (dd, J=5.9, 3.9 Hz, 5H), 3.52 (ddd, J=10.4, 8.4, 2.3 Hz, 1H), 3.32 (s, 5H), 3.02 (t, J=9.4 Hz, 1H), 2.44 (t, J=10.8 Hz, 3H), 2.16 (s, 3H), 2.09 (q, J=5.3, 4.7 Hz, 1H), 1.65 (p, J=10.0 Hz, 1H).

### Example 56: (3S)-N-(4-methyl-3-[2-[(3-methyloxetan-3-yl)amino]-6-(morpholin-4-yl)pyridin-4-yl]phenyl)-3-(2,2,2-trifluoroethyl)pyrrolidine-1-carboxamide

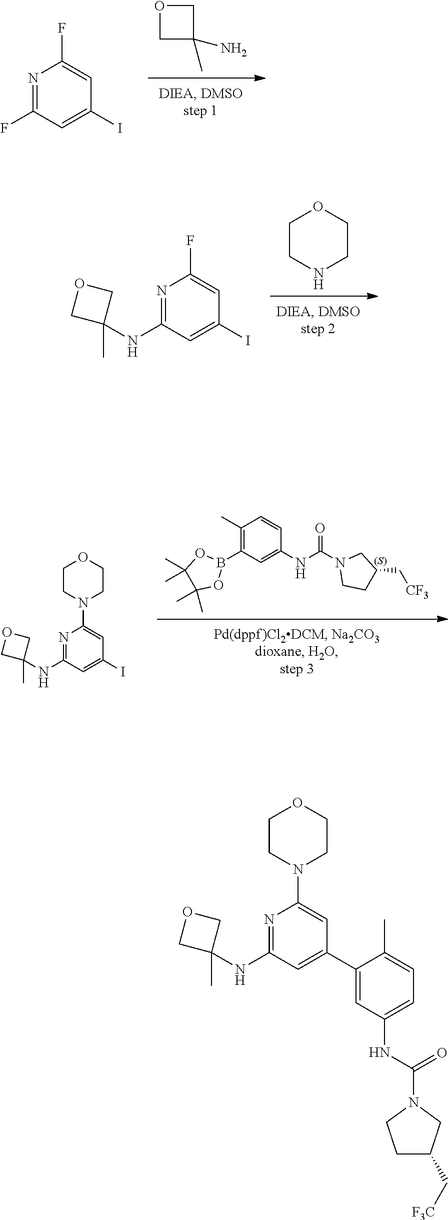

Preparation 56A: 6-fluoro-4-iodo-N-(3-methyloxetan-3-yl)pyridin-2-amine

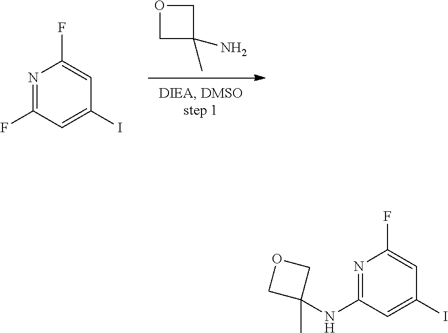

A solution of 2,6-difluoro-4-iodopyridine (500 mg, 2.075 mmol), 3-methyloxetan-3-amine (181 mg, 2.075 mmol) and DIEA (295 mg, 2.282 mmol) in DMSO (5 mL) was stirred for 16 h at 70° C. under nitrogen atmosphere. The resulting mixture was cooled down to room temperature and diluted with water (50 mL) and extracted with EtOAc (3×30 mL). The combined organic layers was washed with brine (100 mL), dried over anhydrous Na₂SO₄. After filtration, the filtrate was concentrated under reduced pressure. The residue was purified by silica gel column chromatography, eluted with EtOAc in PE (50%) to afford 6-fluoro-4-iodo-N-(3-methyloxetan-3-yl)pyridin-2-amine (450 mg, 70%) as an off-white solid. MS ESI calculated for C9H₁₀FIN₂O [M+H]⁺, 309.09, found 308.85. ¹H NMR (400 MHz, DMSO-d₆) δ 7.62 (s, 1H), 6.74-6.73 (m, 1H), 6.59-6.58 (m, 1H), 4.59-4.58 (m, 2H), 4.39-4.38 (m, 2H), 1.58 (s, 3H). ¹⁹F NMR (376 MHz, DMSO-d₆) δ-69.76 (1F).

Preparation 56B: 4-iodo-N-(3-methyloxetan-3-yl)-6-(morpholin-4-yl)pyridin-2-amine

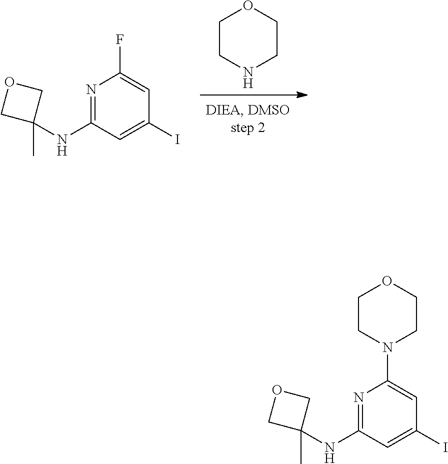 [SMILES: CC1(Nc2cc(I)cc(F)n2)COC1 ; C1COCCN1 ; CC1(Nc2cc(I)cc(N3CCOCC3)n2)COC1]

A solution of 6-fluoro-4-iodo-N-(3-methyloxetan-3-yl)pyridin-2-amine (400 mg, 1.298 mmol), morpholine (124 mg, 1.428 mmol) and DIEA (335 mg, 2.597 mmol) in DMSO (4 mL) was stirred for 16 h at 100° C. under nitrogen atmosphere. The resulting mixture was cooled down to room temperature and diluted with water (50 mL) and extracted with EtOAc (3×30 mL). The combined organic layers was washed with brine (100 mL), dried over anhydrous Na₂SO₄. After filtration, the filtrate was concentrated under reduced pressure. The residue was purified by silica gel column chromatography, eluted with EtOAc in PE (50%) to afford 4-iodo-N-(3-methyloxetan-3-yl)-6-(morpholin-4-yl)pyridin-2-amine (270 mg, 55%) as a light yellow solid. MS ESI calculated for C₁₃H₁₈IN₃O₂[M+H]⁺, 376.21, found 375.90. ¹H NMR (400 MHz, DMSO-d₆) δ 6.90 (s, 1H), 6.25 (s, 1H), 6.20 (s, 1H), 4.63-4.60 (m, 2H), 4.36-4.34 (m, 2H), 3.65-3.62 (m, 4H), 3.31-3.28 (m, 4H), 1.59 (s, 3H).

### Example 56: (3S)-N-(4-methyl-3-[2-[(3-methyloxetan-3-yl)amino]-6-(morpholin-4-yl)pyridin-4-yl]phenyl)-3-(2,2,2-trifluoroethyl)pyrrolidine-1-carboxamide

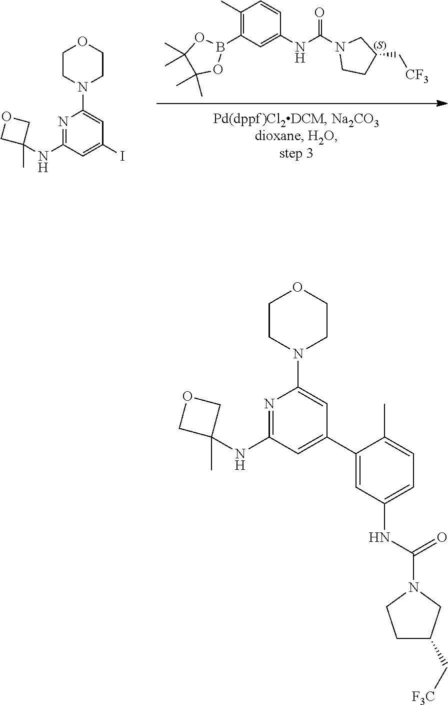

A mixture of 4-iodo-N-(3-methyloxetan-3-yl)-6-(morpholin-4-yl)pyridin-2-amine (200 mg, 0.533 mmol), (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (242 mg, 0.586 mmol), Pd(dppf)Cl₂·CH₂Cl₂ (43 mg, 0.053 mmol), Na₂CO₃ (169 mg, 1.599 mmol), 1,4-dioxane (6 mL) and H₂O (1.5 mL) was stirred for 16 h at 60° C. under nitrogen atmosphere. The reaction mixture was cooled to room temperature and concentrated under reduced pressure. The residue was purified by silica gel column chromatography, eluted with EA/EtOH (3/1) in PE (58%) followed by prep-HPLC with the following conditions: Column: XBridge Prep Phenyl OBD Column, 19×150 mm 5 μm 13 nm; Mobile Phase A: water (10 mmol/L NH₄HCO₃), Mobile Phase B: acetonitrile; Flow rate:20 mL/min; Gradient:40 B to 65 B in 5.5 min; 254/210 nm to afford (3S)-N-(4-methyl-3-[2-[(3-methyloxetan-3-yl)amino]-6-(morpholin-4-yl)pyridin-4-yl]phenyl)-3-(2,2,2-trifluoroethyl)pyrrolidine-1-carboxamide (97 mg, 33%) as an off-white solid. MS ESI calculated for C₂₇H₃₄F₃N₅O₃ [M+H]⁺, 534.26, found 534.25. ¹H NMR (400 MHz, DMSO-d₆) δ 8.12 (s, 1H), 7.42-7.35 (m, 2H), 7.11-7.09 (m, 1H), 6.77 (s, 1H), 5.81 (s, 1H), 5.73 (s, 1H), 4.70-4.69 (m, 2H), 4.40-4.38 (m, 2H), 3.68-3.64 (m, 5H), 3.52-3.50 (m, 1H), 3.35-3.29 (m, 5H), 3.04-2.99 (m, 1H), 2.52-2.41 (m, 3H), 2.16 (s, 3H), 2.06-2.04 (m, 1H), 1.75-1.60 (m, 4H). ¹⁹F NMR (376 MHz, DMSO-d₆) δ-63.37 (3F).

### Example 57: (3S)-N-(4-methyl-3-[2-[(4-methyloxan-4-yl)amino]-6-(morpholin-4-yl)pyridin-4-yl]phenyl)-3-(2,2,2-trifluoroethyl)pyrrolidine-1-carboxamide

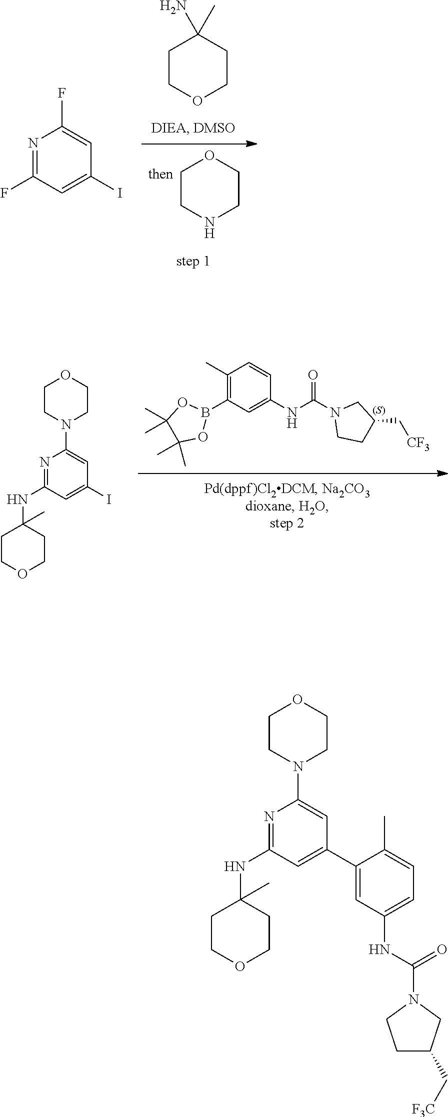

Preparation 57A: 4-iodo-N-(4-methyloxan-4-yl)-6-(morpholin-4-yl)pyridin-2-amine

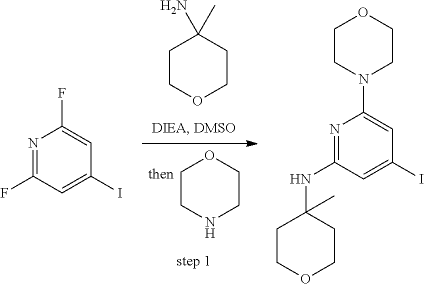

A solution of 2,6-difluoro-4-iodopyridine (200 mg, 0.830 mmol), 4-methyloxan-4-amine (105 mg, 0.913 mmol), DMSO (2 mL) and DIEA (0.32 mL, 2.461 mmol) was stirred for 4 h at 110° C. To this was added morpholine (361 mg, 4.150 mmol). The resulting mixture was stirred for 16 h at 110° C. The mixture was allowed to cool down to room temperature and quenched with water (10 mL). The resulting mixture was extracted with EtOAc (3×10 mL). The combined organic layers were washed with brine (10 mL), dried over anhydrous Na₂SO₄. After filtration, the filtrate was concentrated under reduced pressure. The residue was purified by silica gel column chromatography, eluted with PE/EtOAc (4:1) to afford 4-iodo-N-(4-methyloxan-4-yl)-6-(morpholin-4-yl)pyridin-2-amine (220 mg, 66%) as a yellow solid. MS ESI calculated for C₁₅H₂₂IN₃O₂[M+H]⁺, 404.10, found 404.10. ¹H NMR (300 MHz, CDCl₃-d) δ 6.23 (dd, J=10.0, 1.0 Hz, 2H), 4.05 (s, 1H), 3.86-3.67 (m, 11H), 3.43-3.35 (m, 5H), 2.10 (t, J=12.3 Hz, 3H).

### Example 57: (3S)-N-(4-methyl-3-[2-[(4-methyloxan-4-yl)amino]-6-(morpholin-4-yl)pyridin-4-yl]phenyl)-3-(2,2,2-trifluoroethyl)pyrrolidine-1-carboxamide

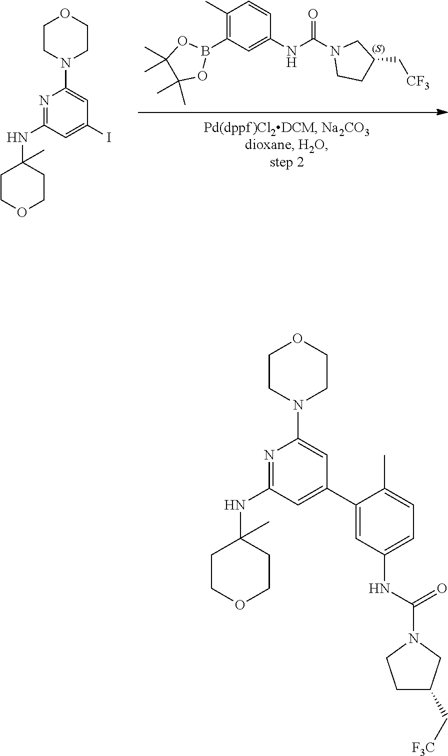

A mixture of 4-iodo-N-(4-methyloxan-4-yl)-6-(morpholin-4-yl)pyridin-2-amine (220 mg, 0.546 mmol), (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (225 mg, 0.546 mmol), 1,4-dioxane (2 mL), H₂O (0.4 mL), Na₂CO₃ (173 mg, 1.637 mmol) and Pd(dppf)Cl₂·CH₂Cl₂ (44 mg, 0.055 mmol) was stirred for 16 h at 60° C. under nitrogen atmosphere. The mixture was allowed to cool down to room temperature. The reaction was quenched by the addition of water (10 mL) at room temperature. The resulting mixture was extracted with EtOAc (3×10 mL). The combined organic layers were washed with brine (10 mL), dried over anhydrous Na₂SO₄. After filtration, the filtrate was concentrated under reduced pressure. The residue was purified by silica gel column chromatography, eluted with PE/EtOAc (1/2) to afford (3S)-N-(4-methyl-3-[2-[(4-methyloxan-4-yl)amino]-6-(morpholin-4-yl)pyridin-4-yl]phenyl)-3-(2,2,2-trifluoroethyl)pyrrolidine-1-carboxamide (158 mg, 52%) as a light yellow solid. MS ESI calculated for C₂₉H₃₈F₃N₅O₃[M+H]⁺, 562.15, found 562.15. ¹H NMR (400 MHz, DMSO-d₆) δ 8.11 (s, 1H), 7.42 (dd, J=8.3, 2.4 Hz, 1H), 7.35 (d, J=2.3 Hz, 1H), 7.09 (d, J=8.4 Hz, 1H), 5.97 (s, 1H), 5.91 (d, J=0.9 Hz, 1H), 5.75 (d, J=1.0 Hz, 1H), 3.72-3.54 (m, 9H), 3.52-3.49 (m, 1H), 3.36-3.32 (m, 5H), 3.02 (t, J=9.5 Hz, 1H), 2.47-2.35 (m, 3H), 2.21 (s, 1H), 2.17 (s, 4H), 2.09 (dd, J=12.4, 6.2 Hz, 1H), 1.73-1.53 (m, 3H), 1.46 (s, 3H). ¹⁹F NMR (376 MHz, DMSO-d₆) δ-63.36 (3F).

### Example 58: (3S)-N-[3-(2-[[1-(hydroxymethyl)cyclopropyl]amino]-6-(morpholin-4-yl)pyridin-4-yl)-4-methylphenyl]-3-(2,2,2-trifluoroethyl)pyrrolidine-1-carboxamide

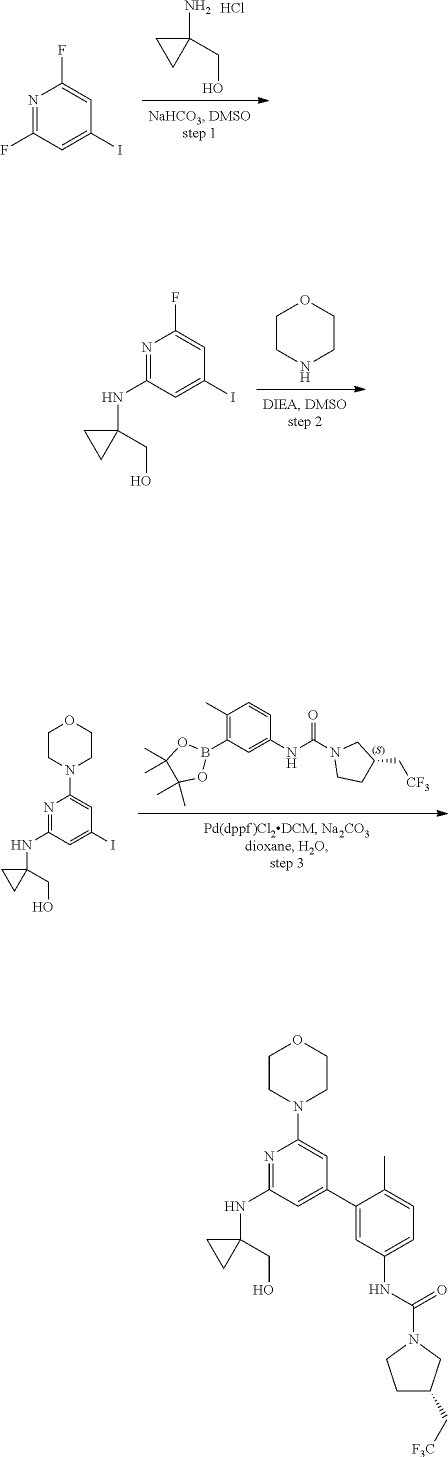

Preparation 58A: [1-[(6-fluoro-4-iodopyridin-2-yl)amino]cyclopropyl]methanol

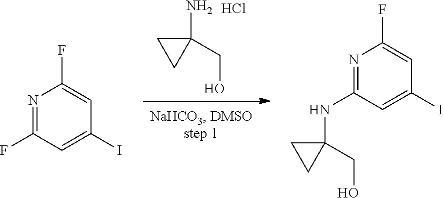 [SMILES: Fc1cc(I)cc(F)n1 ; NC1(CO)CC1 ; Cl ; OCC1(Nc2cc(I)cc(F)n2)CC1]

A mixture of 2,6-difluoro-4-iodopyridine (500 mg, 2.075 mmol), (1-aminocyclopropyl)methanol hydrochloride (282 mg, 2.282 mmol) and NaHCO₃ (523 mg, 6.225 mmol) in DMSO (5 mL) was stirred for 3 h at 100° C. under nitrogen atmosphere. The resulting mixture was cooled down to room temperature and diluted with water (50 mL) and extracted with EtOAc (3×30 mL). The combined organic layers was washed with brine (100 mL), dried over anhydrous Na₂SO₄. After filtration, the filtrate was concentrated under reduced pressure. The residue was purified by silica gel column chromatography, eluted with EtOAc in PE (58%) to afford [1-[(6-fluoro-4-iodopyridin-2-yl)amino]cyclopropyl]methanol (575 mg, 90%) as light yellow oil. MS ESI calculated for C₉H₁₀FIN₂O [M+H]⁺, 309.09, found 308.90. ¹H NMR (400 MHz, DMSO-d₆) δ 7.42 (s, 1H), 6.84 (s, 1H), 6.59 (s, 1H), 4.77 (brs, 1H), 3.48-3.42 (m, 2H), 0.80-0.74 (m, 2H), 0.61-0.55 (m, 2H). ¹⁹F NMR (376 MHz, DMSO-d₆) δ-70.00 (1F).

Preparation 58B: (1-[[4-iodo-6-(morpholin-4-yl)pyridin-2-yl]amino]cyclopropyl)methanol

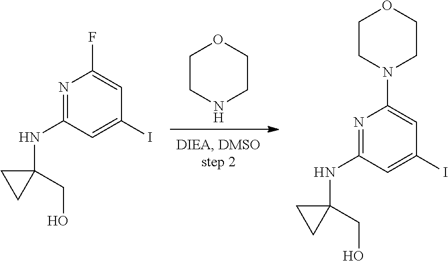 [SMILES: OCC1(Nc2cc(I)cc(F)n2)CC1 ; C1COCCN1 ; OCC1(Nc2cc(I)cc(N3CCOCC3)n2)CC1]

A solution of [1-[(6-fluoro-4-iodopyridin-2-yl)amino]cyclopropyl]methanol (575 mg, 1.866 mmol), morpholine (179 mg, 2.053 mmol) and DIEA (482 mg, 3.733 mmol) in DMSO (6 mL) was stirred for 16 h at 100° C. under nitrogen atmosphere. The resulting mixture was cooled down to room temperature and quenched with water (60 mL) and extracted with EtOAc (3×30 mL). The combined organic layers was washed with brine (100 mL), dried over anhydrous Na₂SO₄. After filtration, the filtrate was concentrated under reduced pressure. The residue was purified by silica gel column chromatography, eluted with EtOAc in PE (76%) to afford (1-[[4-iodo-6-(morpholin-4-yl)pyridin-2-yl]amino]cyclopropyl)methanol (315 mg, 45%) as a light yellow solid. MS ESI calculated for C₁₃H₁₈IN₃O₂ [M+H]⁺, 376.21, found 375.90. ¹H NMR (400 MHz, DMSO-d₆) δ 6.60 (s, 1H), 6.29-6.27 (m, 2H), 4.68 (brs, 1H), 3.64-3.62 (m, 4H), 3.50-3.41 (m, 2H), 3.33-3.30 (m, 4H), 0.75-0.72 (m, 2H), 0.57-0.55 (m, 2H).

### Example 58: (3S)-N-[3-(2-[[1-(hydroxymethyl)cyclopropyl]amino]-6-(morpholin-4-yl)pyridin-4-yl)-4-methylphenyl]-3-(2,2,2-trifluoroethyl)pyrrolidine-1-carboxamide

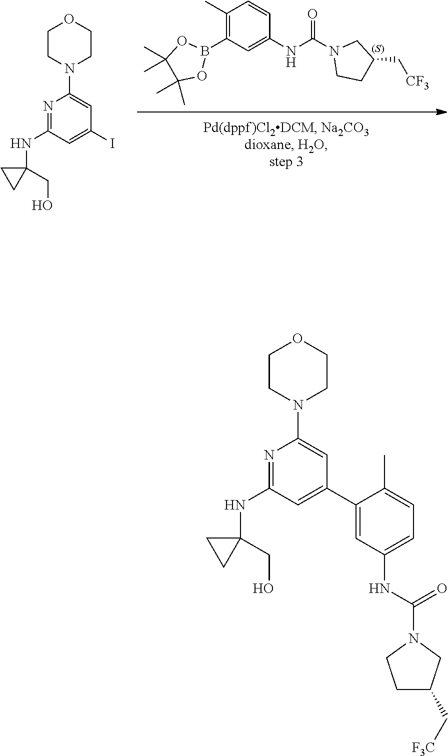

A mixture of (1-[[4-iodo-6-(morpholin-4-yl)pyridin-2-yl]amino]cyclopropyl)methanol (200 mg, 0.533 mmol), (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (242 mg, 0.586 mmol), Pd(dppf)Cl₂·CH₂Cl₂ (43 mg, 0.053 mmol), Na₂CO₃ (169 mg, 1.599 mmol), 1,4-dioxane (6 mL) and water (1.5 mL) was stirred for 16 h at 60° C. under nitrogen atmosphere. The reaction mixture was cooled to room temperature and concentrated under reduced pressure. The residue was purified by silica gel column chromatography, eluted with EA/EtOH (3/1) in PE (60%) followed by prep-HPLC with the following conditions: Column: XBridge Prep Phenyl OBD Column, 19×150 mm Sum 13 nm; Mobile Phase A: water (10 mmol/L NH₄HCO₃), Mobile Phase B: acetonitrile; Flow rate:20 mL/min; Gradient:45 B to 65 B in 5.5 min; 254/210 nm to afford (3S)-N-[3-(2-[[1-(hydroxymethyl)cyclopropyl]amino]-6-(morpholin-4-yl)pyridin-4-yl)-4-methylphenyl]-3-(2,2,2-trifluoroethyl)pyrrolidine-1-carboxamide (151 mg, 51%) as an off-white solid. MS ESI calculated for C₂₇H₃₄F₃N₅O₃ [M+H]⁺, 534.59, found 534.25. ¹H NMR (400 MHz, DMSO-d₆) δ 8.12 (s, 1H), 7.43-7.33 (m, 2H), 7.11-7.09 (m, 1H), 6.43 (s, 1H), 5.85-5.83 (m, 2H), 4.75-4.74 (m, 1H), 3.68-3.66 (m, 5H), 3.52-3.49 (m, 3H), 3.38-3.30 (m, 5H), 3.03-3.02 (m, 1H), 2.52-2.44 (m, 3H), 2.16 (s, 3H), 2.08-2.06 (m, 1H), 1.78-1.65 (m, 1H), 0.76-0.75 (m, 2H), 0.63-0.62 (m, 2H). ¹⁹F NMR (376 MHz, DMSO-d₆) δ-63.36 (3F).

### Examples 59 and 60: (S)-N-(3-(2-(((1R,3S)-3-hydroxy-1-methylcyclobutyl)amino)-6-morpholinopyridin-4-yl)-4-methylphenyl)-3-(2,2,2-trifluoroethyl)pyrrolidine-1-carboxamide, (S)-N-(3-(2-(((1S,3R)-3-hydroxy-1-methylcyclobutyl)amino)-6-morpholinopyridin-4-yl)-4-methylphenyl)-3-(2,2,2-trifluoroethyl)pyrrolidine-1-carboxamide

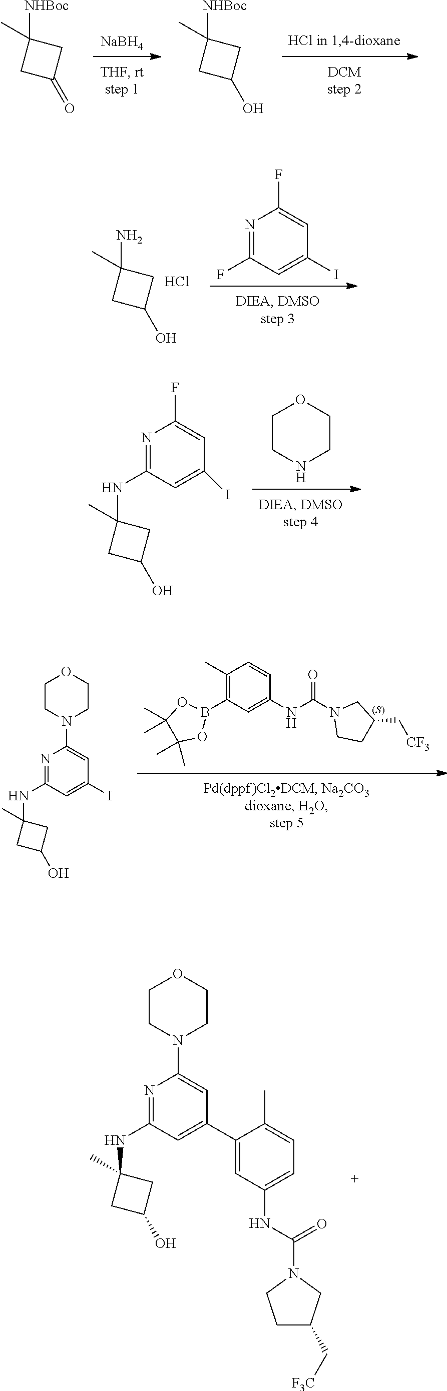

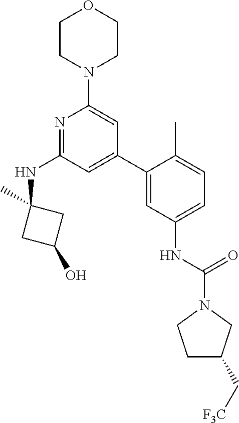 [SMILES: Cc1ccc(NC(=O)N2CC[C@@H](CC(F)(F)F)C2)cc1-c1cc(N[C@]2(C)C[C@H](O)C2)nc(N2CCOCC2)c1]

Preparation 59A tert-buty (3-hydroxy-1-methylcyclobutyl) carbamate

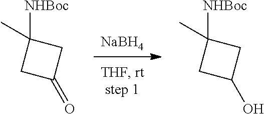

To a solution of tert-butyl N-(1-methyl-3-oxocyclobutyl) carbamate (2.00 g, 10.04 mmol) in THF (20 mL) was added NaBH₄ (0.76 g, 20.07 mmol) dropwise at 0° C. under nitrogen atmosphere. The resulting mixture was stirred for 16 h at room temperature. The reaction was quenched with sat. NH₄C1 (100 mL) at 0° C. The resulting mixture was extracted with EtOAc (3×100 mL). The combined organic layers were washed with brine (50 mL), dried over anhydrous Na₂SO₄. After filtration, the filtrate was concentrated under reduced pressure. The resulting mixture was used in the next step directly without further purification. ¹H NMR (400 MHz, chloroform-d) δ 4.72 (s, 1H), 4.17-4.10 (m, 2H), 1.99-1.86 (m, 2H), 1.30 (d, J=20.8 Hz, 9H).

Preparation 59B: 3-amino-3-methylcyclobutan-1-ol hydrochloride

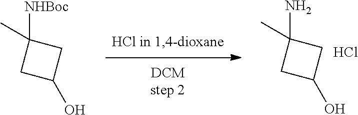

To a solution tert-butyl (3-hydroxy-1-methylcyclobutyl) carbamate (1.50 g, 7.46 mmol) in DCM (4 mL) was added HCl (gas) in 1,4-dioxane (2 mL, 65.82 mmol, 4 M) at 0° C. The resulting mixture was stirred for 1 h at room temperature. The resulting mixture was concentrated under reduced pressure. The resulting mixture was used in the next step directly without further purification. ¹H NMR (400 MHz, DMSO-d₆) δ 8.37 (s, 2H), 4.43-4.35 (m, 1H), 4.04 (p, J=7.1 Hz, 1H), 2.26 (ddt, J=9.3, 7.1, 2.3 Hz, 2H), 2.17-2.08 (m, 2H), 1.33 (s, 3H).

Preparation 59C: 3-((6-fluoropyridin-2-yl)amino)-3-methylcyclobutan-1-ol

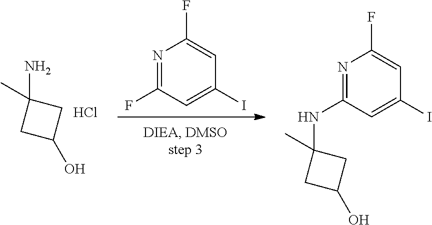

To a solution of 3-amino-3-methylcyclobutan-1-ol hydrochloride (1.3 g, 9.489 mmol) in DMSO (13 mL) were added 2,6-difluoro-4-iodopyridine (1.37 g, 5.67 mmol) and DIEA (6.10 g, 47.23 mmol) at room temperature under. The resulting mixture was stirred for 16 h at 110° C. The reaction was quenched by the addition of water (130 mL) at room temperature. The resulting mixture was extracted with EtOAc (3×100 mL). The combined organic layers were washed with brine (200 mL), dried over anhydrous Na₂SO₄. After filtration, the filtrate was concentrated under reduced pressure. The residue was purified by silica gel column chromatography, eluted with PE/EtOAc (3:1) to afford 3-((6-fluoropyridin-2-yl)amino)-3-methylcyclobutan-1-ol (800 mg, 26%) as a light yellow oil. MS ESI calculated for C₁₀H₁₃FN₂O [M+H]⁺, 323.00, found 323.05. ¹H NMR (400 MHz, chloroform-d) δ 6.55-6.45 (m, 2H), 4.63 (d, J=53.3 Hz, 1H), 4.23 (p, J=6.9 Hz, 1H), 4.13-4.03 (m, 1H), 2.51-2.43 (m, 2H), 2.37-2.25 (m, 2H), 1.34-1.22 (m, 3H).

Preparation 59D: 3-((4-iodo-6-morpholinopyridin-2-yl)amino)-3-methylcyclobutan-1-ol

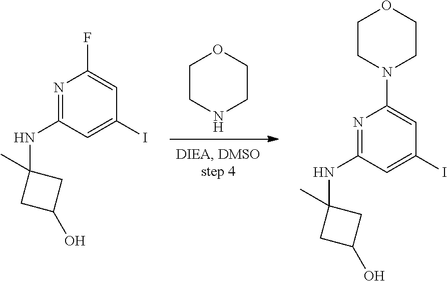 [SMILES: CC1(Nc2cc(I)cc(F)n2)CC(O)C1 ; C1COCCN1 ; CC1(Nc2cc(I)cc(N3CCOCC3)n2)CC(O)C1]

To a solution of 3-((6-fluoro-4-iodopyridin-2-yl)amino)-3-methylcyclobutan-1-ol (980 mg, 3.043 mmol) in DMSO (10 mL) was added DIEA (786 mg, 6.085 mmol) at room temperature. The resulting mixture was stirred for 16 h at 110° C. The reaction was quenched with water (100 mL). The resulting mixture was extracted with EtOAc (3×50 mL). The combined organic layers were washed with brine (100 mL), dried over anhydrous Na₂SO₄. After filtration, the filtrate was concentrated under reduced pressure. The residue was purified by silica gel column chromatography, eluted with PE/EtOAc (1:1) to afford 3-((4-iodo-6-morpholinopyridin-2-yl)amino)-3-methylcyclobutan-1-ol (200 mg, 17%) as a light yellow oil. MS ESI calculated for C₁₄H₂₀IN₃O₂[M+H]⁺, 390.06, found 389.95. ¹H NMR (400 MHz, DMSO-d₆) δ 6.17 (d, J=1.1 Hz, 1H), 6.14 (d, J=1.0 Hz, 1H), 4.03 (q, J=7.1 Hz, 3H), 3.90 (t, J=7.3 Hz, 1H), 3.64 (dd, J=5.8, 3.9 Hz, 4H), 2.39 (ddd, J=12.0, 6.0, 2.6 Hz, 2H), 2.23-2.20 (m, 2H), 1.21 (s, 3H).

### Examples 59 and 60: (S)-N-(3-(2-(((1R,3S)-3-hydroxy-1-methylcyclobutyl)amino)-6-morpholinopyridin-4-yl)-4-methylphenyl)-3-(2,2,2-trifluoroethyl)pyrrolidine-1-carboxamide, (S)-N-(3-(2-(((1S,3R)-3-hydroxy-1-methylcyclobutyl)amino)-6-morpholinopyridin-4-yl)-4-methylphenyl)-3-(2,2,2-trifluoroethyl)pyrrolidine-1-carboxamide

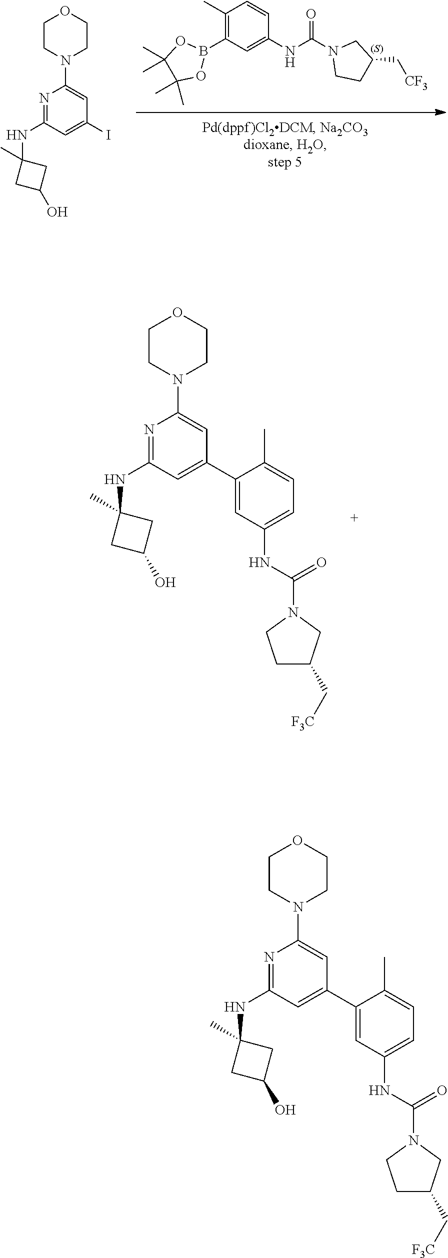

A mixture of 3-((4-iodo-6-morpholinopyridin-2-yl)amino)-3-methylcyclobutan-1-ol (200 mg, 0.514 mmol), (3S)-N-[4-methyl-3-(4,4,5,5-tetramethyl-1,3,2-dioxaborolan-2-yl)phenyl]-3-(2,2,2-trifluoroethyl) pyrrolidine-1-carboxamide (212 mg, 0.514 mmol), Pd(dppf)Cl₂·CH₂Cl₂ (42 mg, 0.051 mmol) and Na₂CO₃ (163 mg, 1.541 mmol) in dioxane (2 mL) and H₂O (0.2 mL) was stirred for 3 h at 80° C. under nitrogen atmosphere. The reaction mixture was concentrated under reduced pressure. The residue was purified by silica gel column chromatography, eluted with PE/EtOAc (1/2). The resulting mixture was resolved by chiral HPLC with following conditions: Column: CHIRALPAK IE, 2*25 cm, 5 um; Mobile Phase A:Hex (0.5% 2 M NH₃-MeOH), Mobile Phase B:EtOH; Flow rate:18 mL/min; A:B=70:30; 220/254 nm to afforde 14.7 mg (9%) as a light blue solid. MS ESI calculated for C₂₈H₃₆F₃N₅O₃[M+H]⁺, 548.28, found 548.25. ¹H NMR (400 MHz, DMSO-d₆) δ 8.10 (s, 1H), 7.48-7.32 (m, 2H), 7.09 (d, J=8.3 Hz, 1H), 6.24 (s, 1H), 5.73 (d, J=28.8 Hz, 2H), 4.91 (d, J=5.8 Hz, 1H), 4.16 (h, J=6.7 Hz, 1H), 3.75-3.60 (m, 5H), 3.53 (ddd, J=10.4, 8.2, 2.3 Hz, 1H), 3.36 (d, J=9.6 Hz, 4H), 3.32-3.25 (m, 1H), 3.02 (t, J=9.4 Hz, 1H), 2.63 (ddd, J=10.0, 7.3, 2.6 Hz, 2H), 2.43 (dd, J=15.0, 7.1 Hz, 3H), 2.17 (s, 4H), 1.89-1.77 (m, 2H), 1.77-1.59 (m, 1H), 1.49 (s, 3H). ¹⁹F NMR (376 MHz, DMSO-d₆) δ-63.36, and 50.5 mg (30%) as a light blue solid. MS ESI calculated for C₂₈H₃₆F₃N₅O₃[M+H]⁺, 548.28, found 548.25. ¹H NMR (300 MHz, DMSO-d₆) δ 8.11 (s, 1H), 7.49-7.30 (m, 2H), 7.09 (d, J=8.3 Hz, 1H), 6.42 (s, 1H), 5.72 (d, J=19.3 Hz, 2H), 4.93 (d, J=6.2 Hz, 1H), 4.03 (h, J=7.1 Hz, 1H), 3.80-3.62 (m, 5H), 3.53 (ddd, J=10.5, 8.3, 2.4 Hz, 1H), 3.39 (s, 1H), 3.37-3.21 (m, 3H), 3.03 (t, J=9.3 Hz, 1H), 2.50-2.34 (m, 5H), 2.17 (s, 3H), 2.05 (t, J=10.2 Hz, 3H), 1.85-1.55 (m, 1H), 1.42 (s, 3H). ¹⁹F NMR (282 MHz, DMSO-d₆) δ-63.36, −63.43.

### Example 61: (3S)-N-[3-(2-[[(2R,3R)-3-hydroxybutan-2-yl]amino]-6-(morpholin-4-yl)pyridin-4-yl)-4-methylphenyl]-3-(2,2,2-trifluoroethyl)pyrrolidine-1-carboxamide

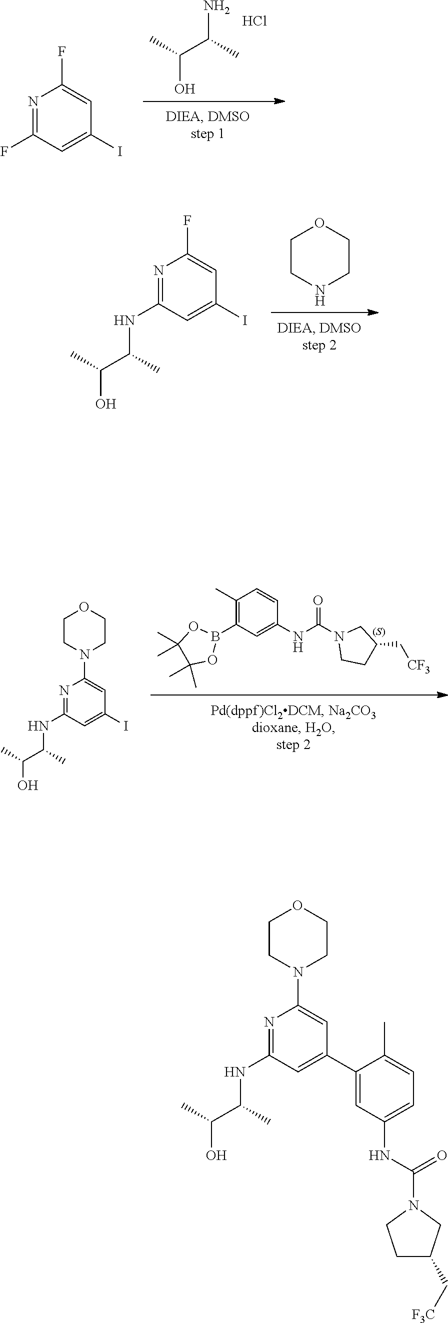

Preparation 61A: (2R,3R)-3-[(6-fluoro-4-iodopyridin-2-yl)amino]butan-2-ol

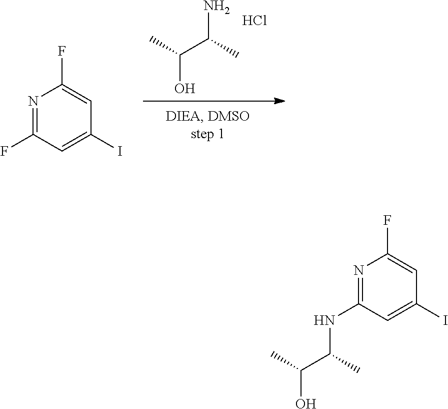

A solution of 2,6-difluoro-4-iodopyridine (300 mg, 1.245 mmol), DMSO (3 mL), (2R,3R)-3-aminobutan-2-ol (133 mg, 1.494 mmol) and DIEA (804 mg, 6.225 mmol) was stirred for 2 h at 70° C. The resulting mixture was quenched with H₂O (100 mL) and extracted with EtOAc (4×50 mL). The combined organic layers were washed with brine (4×50 mL), dried over anhydrous Na₂SO₄. After filtration, the filtrate was concentrated under reduced pressure. The residue was purified by silica gel column chromatography, eluted with PE/EtOAc (1:1) to afford (2R,3R)-3-[(6-fluoro-4-iodopyridin-2-yl)amino]butan-2-ol (172 mg, 44%) as a light yellow oil. MS ESI calculated for C₉H₁₂FIN₂O [M+H]⁺, 311.11, found 310.90. ¹H NMR (400 MHz, chloroform-d) δ 6.65 (s, 1H), 6.52 (m, 1H), 4.78 (s, 1H), 3.80 (m, 2H), 2.19 (s, 1H), 1.32-1.19 (m, 6H).

Preparation 61B: (2R,3R)-3-[[4-iodo-6-(morpholin-4-yl)pyridin-2-yl]amino]butan-2-ol

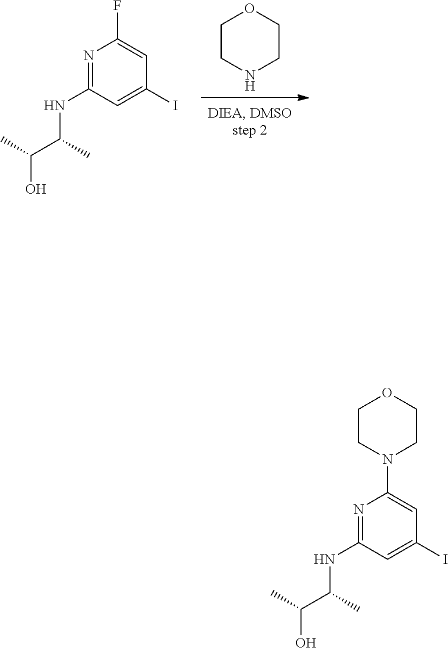 [SMILES: C[C@@H](O)[C@@H](C)Nc1cc(I)cc(F)n1 ; C1COCCN1 ; C[C@@H](O)[C@@H](C)Nc1cc(I)cc(N2CCOCC2)n1]

A mixture of (2R,3R)-3-[(6-fluoro-4-iodopyridin-2-yl)amino]butan-2-ol (110 mg, 0.355 mmol), DMSO (2 mL), morpholine (102 mg, 1.171 mmol) and DIEA (55 mg, 0.426 mmol) was stirred for 16 h at 70° C. The resulting mixture was quenched with H₂O (100 mL) and extracted with EtOAc (4×50 mL). The combined organic layers were washed with brine (4×50 mL), dried over anhydrous Na₂SO₄. After filtration, the filtrate was concentrated under reduced pressure. The residue was purified by silica gel column chromatography, eluted with 39% EtOAc in PE to afford (2R,3R)-3-[[4-iodo-6-(morpholin-4-yl)pyridin-2-yl]amino]butan-2-ol (110 mg, 67%) as a dark yellow oil. MS ESI calculated for C₁₃H₂₀IN₃O₂[M+H]⁺, 379.06, found 379.00. C₁₃H₂₀IN₃O₂: ¹H NMR (400 MHz, chloroform-d) δ 7.28 (s, 1H), 6.28 (s, 1H), 6.24 (s, 1H), 4.44-4.20 (m, 1H), 3.79 (s, 4H), 3.72 (m, 1H), 3.43 (s, 4H), 2.07 (s, 1H), 1.29-1.20 (m, 6H).

### Example 61: (3S)-N-[3-(2-[[(2R,3R)-3-hydroxybutan-2-yl]amino]-6-(morpholin-4-yl)pyridin-4-yl)-4-methylphenyl]-3-(2,2,2-trifluoroethyl)pyrrolidine-1-carboxamide

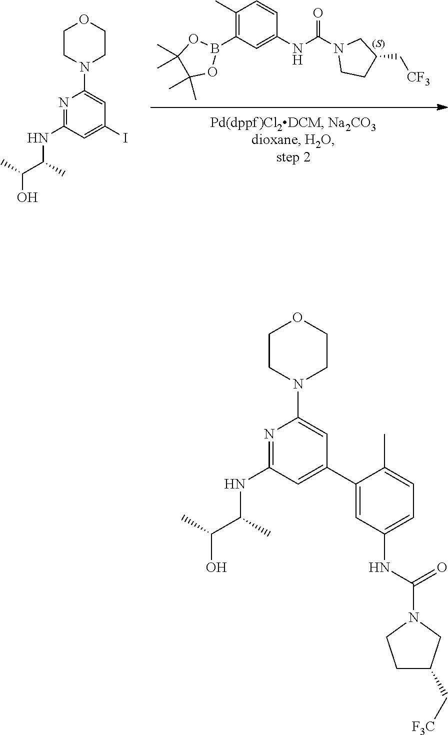

A mixture of (2R,3R)-3-[[4-iodo-6-(morpholin-4-yl)pyridin-2-yl]amino]butan-2-ol (110 mg, 0.292 mmol) and (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (120 mg, 0.292 mmol), 1,4-dioxane (2 mL), H₂O (0.5 mL), Na₂CO₃ (93 mg, 0.875 mmol) and Pd(dppf)Cl₂·CH₂Cl₂ (24 mg, 0.029 mmol) was stirred for 2 h at 80° C. under nitrogen atmosphere. The resulting mixture was concentrated under reduced pressure. The residue was purified by silica gel column chromatography, eluted with EtOAc followed by reverse phase flash with the following conditions (column, C₁₈ silica gel; mobile phase, MeCN in water, 5% to 95% gradient in 10 min; detector, UV 254 nm) to afford (3S)-N-[3-(2-[[(2R,3R)-3-hydroxybutan-2-yl]amino]-6-(morpholin-4-yl)pyridin-4-yl)-4-methylphenyl]-3-(2,2,2-trifluoroethyl)pyrrolidine-1-carboxamide (59 mg, 38%) as a white solid. MS ESI calculated for C₂₇H₃₆F₃N₅O₃ [M+H]⁺, 536.61, found 536.15. ¹H NMR (300 MHz, DMSO-d₆) δ 8.11 (s, 1H), 7.41 (m, 1H), 7.33 (s, 1H), 7.09 (s, 1H), 5.86 (s, 1H), 5.79 (s, 1H), 5.73 (s, 1H), 4.59 (s, 1H), 3.87 (s, 1H), 3.78-3.62 (m, 6H), 3.57-3.47 (m, 1H), 3.37 (sz, 4H), 3.31-3.25 (m, 1H), 3.02 (m, 1H), 2.50 (s, 3H), 2.16 (s, 3H), 2.12-1.97 (m, 1H), 1.66 (m, 1H), 1.06 (m, 6H). ¹⁹F NMR (376 MHz, DMSO-d₆) δ-63.4 (3F).

### Example 62: (3S)-N-(3-[2-[(1-cyanopropan-2-yl)amino]-6-(morpholin-4-yl)pyridin-4-yl]-4-methylphenyl)-3-(2,2,2-trifluoroethyl)pyrrolidine-1-carboxamide

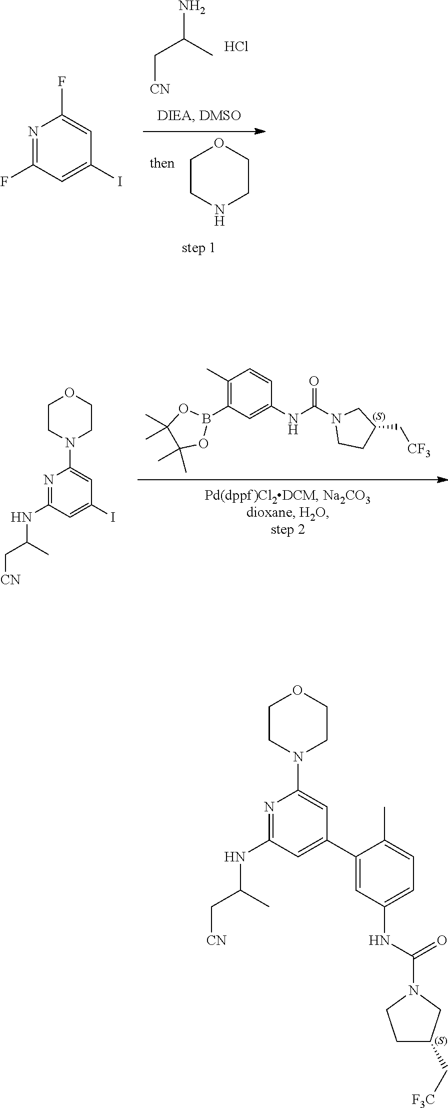

Preparation 62A:3-[[4-iodo-6-(morpholin-4-yl)pyridin-2-yl]amino]butanenitrile

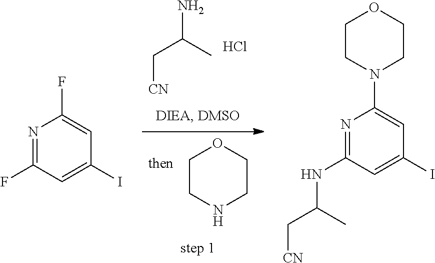

A solution of 2,6-difluoro-4-iodopyridine (300 mg, 1.245 mmol), DMSO (4 mL), 3-aminobutanenitrile hydrochloride (165 mg, 1.369 mmol) and DIEA (354 mg, 2.739 mmol) was stirred for 3 h at 70° C. To above stirred mixture was added morpholine (357 mg, 4.097 mmol). The resulting mixture was stirred for 16 h at 100° C. The resulting mixture was diluted with water (50 mL). The resulting mixture was extracted with EtOAc (3×50 mL). The combined organic layers were washed with brine (2×30 mL), dried over anhydrous Na₂SO₄. After filtration, the filtrate was concentrated under reduced pressure. The residue was purified by silica gel column chromatography, eluted with PE/EtOAc/EtOH=(4/3/1) to afford 3-[[4-iodo-6-(morpholin-4-yl)pyridin-2-yl]amino]butanenitrile (351 mg, 75%) as off-white solid. MS ESI calculated for C₁₃H₁₇IN₄O [M+H]⁺, 373.04, found 372.90. ¹H NMR (300 MHz, chloroform-d) δ 6.45-6.40 (m, 1H), 6.23-6.22 (m, 1H), 4.25-4.22 (m, 1H), 3.84-3.78 (m, 5H), 3.54-3.37 (m, 4H), 2.84-2.64 (m, 2H), 1.47-1.42 (m, 3H).

### Example 62: (3S)-N-(3-[2-[(1-cyanopropan-2-yl)amino]-6-(morpholin-4-yl)pyridin-4-yl]-4-methylphenyl)-3-(2,2,2-trifluoroethyl)pyrrolidine-1-carboxamide

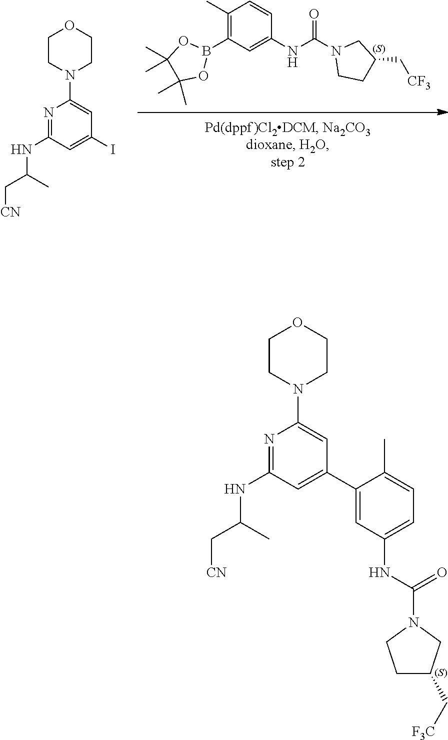

A mixture of 3-[[4-iodo-6-(morpholin-4-yl)pyridin-2-yl]amino]butanenitrile (150 mg, 0.403 mmol), (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (166 mg, 0.403 mmol), Na₂CO₃ (128 mg, 1.209 mmol), dioxane (4 mL), H₂O (1 mL) and Pd(dppf)Cl₂·CH₂Cl₂ (33 mg, 0.040 mmol) was stirred for 16 h at 60° C. under nitrogen atmosphere. The resulting mixture was diluted with water (50 mL) and extracted with EtOAc (3×50 mL). The combined organic layers were washed with brine (2×40 mL), dried over anhydrous Na₂SO₄. After filtration, the filtrate was concentrated under reduced pressure. The residue was purified by silica gel column chromatography, eluted with hexane/EtOAc/EtOH=4/3/1 followed by reverse flash chromatography with the following conditions: column, C₁₈ silica gel; mobile phase, MeCN in water, 5% to 95% gradient in 10 min; detector, UV 254 nm to afford (3S)-N-(3-[2-[(1-cyanopropan-2-yl)amino]-6-(morpholin-4-yl)pyridin-4-yl]-4-methylphenyl)-3-(2,2,2-trifluoroethyl)pyrrolidine-1-carboxamide (149 mg, 69% as a white solid. MS ESI calculated for C₂₇H₃₃F₃N₆O₂[M+H]⁺, 531.26, found 531.15. ¹H NMR (300 MHz, DMSO-d₆) δ 8.01 (s, 1H), 7.43-7.33 (m, 2H), 7.11-7.08 (m, 1H), 6.53-6.51 (m, 1H), 5.82-5.81 (m, 2H), 4.16-4.15 (m, 1H), 3.68-3.49 (m, 5H), 3.52-3.49 (m, 1H), 3.39-3.33 (m, 4H), 3.04-3.02 (m, 1H), 2.98-2.72 (m, 2H), 2.50-2.42 (m, 3H), 2.18-2.01 (m, 5H), 2.71-2.59 (m, 1H), 1.29-1.22 (m, 3H). ¹⁹F NMR (282 MHz, DMSO-d₆) δ-63.36 (3F).

### Examples 63 and 64: (S)-N-(3-(2-(((1S,4R)-4-hydroxy-1-methylcyclohexyl)amino)-6-morpholinopyridin-4-yl)-4-methylphenyl)-3-(2,2,2-trifluoroethyl)pyrrolidine-1-carboxamide, (S)-N-(3-(2-(((1R,4S)-4-hydroxy-1-methylcyclohexyl)amino)-6-morpholinopyridin-4-yl)-4-methylphenyl)-3-(2,2,2-trifluoroethyl)pyrrolidine-1-carboxamide

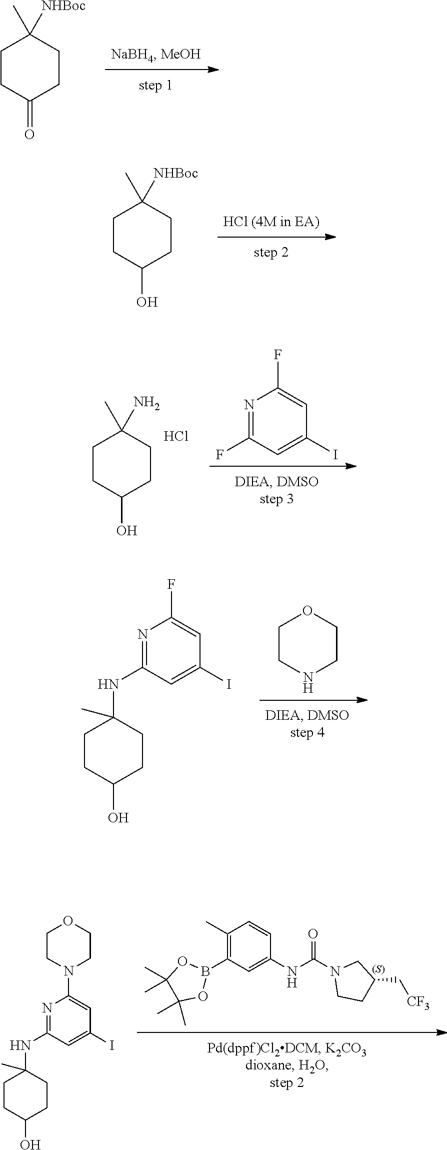

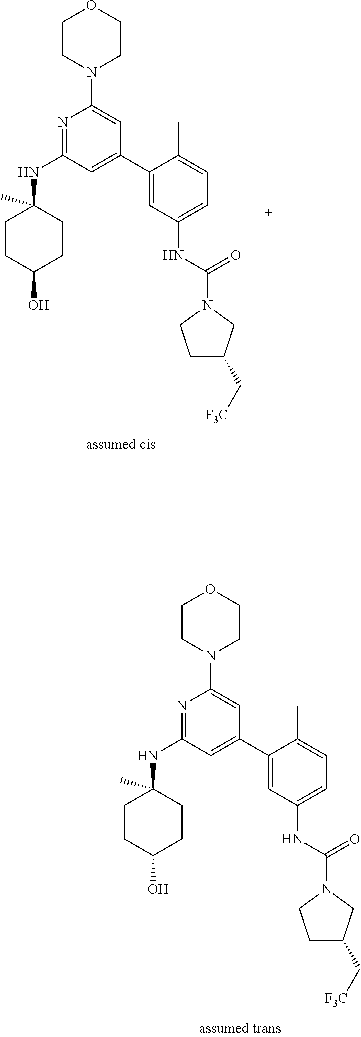 [SMILES: Cc1ccc(NC(=O)N2CC[C@@H](CC(F)(F)F)C2)cc1-c1cc(N[C@]2(C)CC[C@H](O)CC2)nc(N2CCOCC2)c1 ; Cc1ccc(NC(=O)N2CC[C@@H](CC(F)(F)F)C2)cc1-c1cc(N[C@]2(C)CC[C@@H](O)CC2)nc(N2CCOCC2)c1]

Preparation 63A: tert-butyl N-(4-hydroxy-1-methylcyclohexyl)carbamate

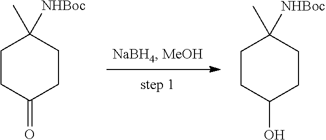 [SMILES: CC1(NC(=O)OC(C)(C)C)CCC(=O)CC1 ; CC1(NC(=O)OC(C)(C)C)CCC(O)CC1]

To a solution of tert-butyl N-(1-methyl-4-oxocyclohexyl)carbamate (700 mg, 3.080 mmol) in MeOH (10 mL) was added NaBH₄ (174 mg, 4.619 mmol) in ports at room temperature. The mixture was stirred at room temperature for 2 h. The reaction was quenched by the addition of water (50 mL) at 0° C. The resulting mixture was extracted with EtOAc (3×50 mL). The combined organic layers were washed with brine (3×50 mL), dried over anhydrous Na₂SO₄.

After filtration, the filtrate was concentrated under reduced pressure. This resulted in tert-butyl N-(4-hydroxy-1-methylcyclohexyl)carbamate (600 mg, 85%) as colorless oil. MS ESI calculated for C₁₂H₂₃NO₃ [M+H]⁺, 230.17 found N/A. ¹H NMR (400 MHz, chloroform-d) δ 3.65 (td, J=10.1, 4.8 Hz, 1H), 1.47 (s, 3H), 1.46-1.40 (m, 8H), 1.44 (s, 9H).

Preparation 63B: 4-amino-4-methylcyclohexan-1-ol hydrochloride

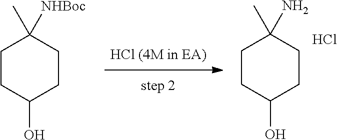

To a solution of tert-butyl N-(4-hydroxy-1-methylcyclohexyl)carbamate (600 mg, 2.616 mmol) in EtOAc (5 mL) was added HCl (gas) in EtOAc (10 mL) at room temperature. The mixture was stirred at room temperature for 2 h. The resulting mixture was concentrated under reduced pressure. This resulted in 4-amino-4-methylcyclohexan-1-ol hydrochloride (400 mg, 92%) as a white solid. The crude product was used in the next step directly without further purification. MS ESI calculated for C₇H₁₅NO [M+H]⁺, 130.12 found N/A. ¹H NMR (400 MHz, methanol-d₄) δ 3.84 (td, J=6.1, 3.1 Hz, 1H), 1.93-1.66 (m, 8H), 1.37 (s, 3H).

Preparation 63C: 4-[(6-fluoro-4-iodopyridin-2-yl)amino]-4-methylcyclohexan-1-ol

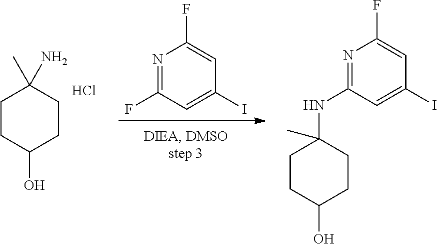

To a solution of 4-(6-fluoro-4-iodopyridin-2-yl)morpholine (744 mg, 2.415 mmol) and 4-amino-4-methylcyclohexan-1-ol hydrochloride (500 mg, 3.018 mmol) in DMSO (10 mL) was added DIEA (1.95 g, 15.091 mmol) at room temperature. The mixture was stirred at 100° C. for 3 h. The mixture was allowed to cool down to room temperature. The reaction was quenched by the addition of water (50 mL) at room temperature. The resulting mixture was extracted with EtOAc (3×50 mL). The combined organic layers were washed with brine (3×50 mL), dried over anhydrous Na₂SO₄. After filtration, the filtrate was concentrated under reduced pressure. MS ESI calculated for C₁₂H₁₆FIN₂O [M+H]⁺, 351.03, found 350.95. ¹H NMR (400 MHz, chloroform-d) δ 6.67-6.61 (m, 1H), 6.51 (dd, J=2.9, 1.0 Hz, 1H), 3.69 (td, J=9.3, 4.2 Hz, 1H), 2.33-2.25 (m, 2H), 1.94-1.78 (m, 3H), 1.49 (d, J=20.6 Hz, 3H), 1.44 (s, 3H).

Preparation 63D: 4-[[4-iodo-6-(morpholin-4-yl)pyridin-2-yl]amino]-4-methylcyclohexan-1-ol

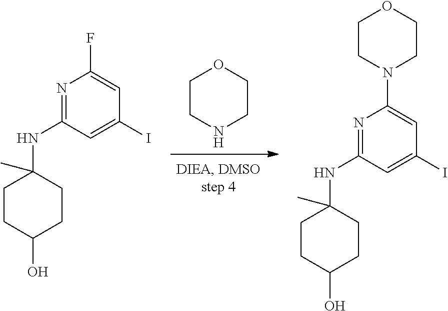 [SMILES: CC1(Nc2cc(I)cc(F)n2)CCC(O)CC1 ; C1COCCN1 ; CC1(Nc2cc(I)cc(N3CCOCC3)n2)CCC(O)CC1]

To a solution of 4-[(6-fluoro-4-iodopyridin-2-yl)amino]-4-methylcyclohexan-1-ol (130 mg, 0.371 mmol) and morpholine (48 mg, 0.557 mmol) in DMSO (5 mL) was added DIEA (96 mg, 0.742 mmol) at room temperature. The mixture was stirred at 110° C. for 16 h. The resulting solution was purified by Prep-HPLC with the following conditions (Column: XBridge Shield RP18 OBD Column, 30*150 mm, 5 um; Mobile Phase A: water (10 mmoL/L NH₄HCO₃), Mobile Phase B: CH₃CN; Flow rate:60 mL/min; Gradient:35 B to 75 B in 25 min) to afford 4-[[4-iodo-6-(morpholin-4-yl)pyridin-2-yl]amino]-4-methylcyclohexan-1-ol (80 mg, 52%) as a light yellow solid. MS ESI calculated for C₁₆H₂₄IN₃O₂[M+H]⁺, 418.09, found 418.00. ¹H NMR (400 MHz, chloroform-d) δ 6.60-6.44 (m, 1H), 6.32 (dd, J=9.8, 1.2 Hz, 1H), 3.85 (q, J=3.9, 2.7 Hz, 5H), 3.54 (dd, J=5.7, 3.8 Hz, 4H), 2.18 (d, J=13.6 Hz, 1H), 1.99-1.76 (m, 3H), 1.70-1.44 (m, 2H), 1.42 (s, 3H), 1.32-1.30 (m, 2H).

### Examples 63 and 64: (S)-N-(3-(2-(((1S,4R)-4-hydroxy-1-methylcyclohexyl)amino)-6-morpholinopyridin-4-yl)-4-methylphenyl)-3-(2,2,2-trifluoroethyl)pyrrolidine-1-carboxamide, (S)-N-(3-(2-(((1R,4S)-4-hydroxy-1-methylcyclohexyl)amino)-6-morpholinopyridin-4-yl)-4-methylphenyl)-3-(2,2,2-trifluoroethyl)pyrrolidine-1-carboxamide

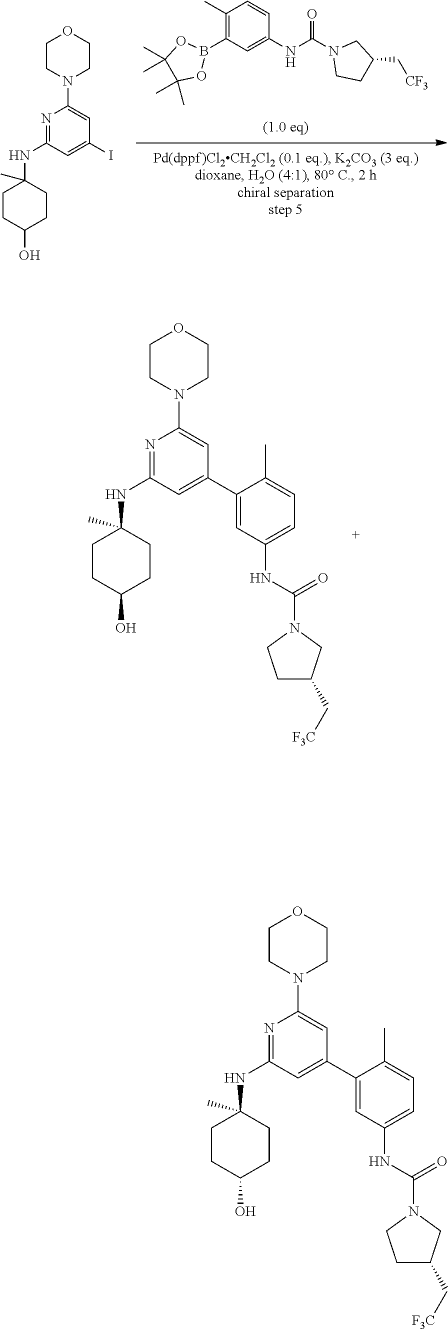 [SMILES: CC1(Nc2cc(I)cc(N3CCOCC3)n2)CCC(O)CC1 ; Cc1ccc(NC(=O)N2CC[C@@H](CC(F)(F)F)C2)cc1B1OC(C)(C)C(C)(C)O1 ; Cc1ccc(NC(=O)N2CC[C@@H](CC(F)(F)F)C2)cc1-c1cc(N[C@]2(C)CC[C@H](O)CC2)nc(N2CCOCC2)c1 ; Cc1ccc(NC(=O)N2CC[C@@H](CC(F)(F)F)C2)cc1-c1cc(N[C@]2(C)CC[C@@H](O)CC2)nc(N2CCOCC2)c1]

To a solution of 4-[[4-iodo-6-(morpholin-4-yl)pyridin-2-yl]amino]-4-methylcyclohexan-1-ol (130 mg, 0.312 mmol) and (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (128 mg, 0.312 mmol) in 1,4-dioxane (10 mL) and H₂O (1 mL) were added Pd(dppf)Cl₂·CH₂Cl₂ (25 mg, 0.031 mmol) and K₂CO₃ (129 mg, 0.935 mmol) at room temperature. The mixture was stirred at 80° C. for 2 h under nitrogen atmosphere. The resulting mixture was concentrated under vacuum. The crude product was purified by Prep-HPLC with the following conditions (Column: XBridge Shield RP18 OBD Column, 30*150 mm, 5 um; Mobile Phase A: water (10 mmoL/L NH₄HCO₃), Mobile Phase B: CH₃CN; Flow rate:60 mL/min; Gradient:35 B to 75 B in 25 min;) to afford racemic (40 mg) as a white solid. The mixture (40 mg) was separated by Chiral-HPLC with the following conditions: Column: CHIRALPAK IH, 2.0*25 cm, 5 um; Mobile Phase A: MeOH (0.1% DEA), Mobile Phase B: CO₂; Flow rate: 50 mL/min; Gradient: 50% B. The first peak afforded 8 mg (4%) as a white solid. MS ESI calculated for C₃₀H₄₀F₃N₅O₃ [M+H]⁺, 576.31, found 576.15. ¹H NMR (400 MHz, methanol-d₄) δ 7.27 (dt, J=4.1, 2.4 Hz, 2H), 7.14 (d, J=8.9 Hz, 1H), 5.87 (d, J=16.9 Hz, 2H), 3.84-3.75 (m, 6H), 3.63 (ddd, J=10.5, 8.4, 2.3 Hz, 1H), 3.44 (h, J=6.5 Hz, 5H), 3.18-3.09 (m, 1H), 2.55 (s, 1H), 2.47-2.32 (m, 2H), 2.23 (s, 4H), 2.05 (t, J=9.4 Hz, 2H), 1.97-1.67 (m, 5H), 1.54 (q, J=8.4, 7.1 Hz, 2H), 1.49 (s, 3H). ¹⁹F NMR (376 MHz, chloroform-d) 6-66.49 (3F). The second peak afforded 33 mg (18%) as a white solid. MS ESI calculated for C₃₀H₄₀F₃N₅O₃ [M+H]⁺, 576.31, found 576.15. ¹H NMR (400 MHz, methanol-d₄) δ 7.31-7.25 (m, 2H), 7.15 (d, J=9.0 Hz, 1H), 5.93 (s, 1H), 5.84 (s, 1H), 3.84-3.75 (m, 5H), 3.68-3.53 (m, 2H), 3.49-3.38 (m, 5H), 3.13 (t, J=9.7 Hz, 1H), 2.65-2.30 (m, 5H), 2.24 (s, 4H), 1.97-1.67 (m, 3H), 1.68-1.53 (m, 2H), 1.46 (s, 3H), 1.44-1.29 (m, 2H). ¹⁹F NMR (376 MHz, chloroform-d) δ-66.48 (3F).

### Example 65: (3S)-N-(3-[2-[(1,3-dihydroxypropan-2-yl)amino]-6-(morpholin-4-yl)pyridin-4-yl]-4-methylphenyl)-3-(2,2,2-trifluoroethyl)pyrrolidine-1-carboxamide

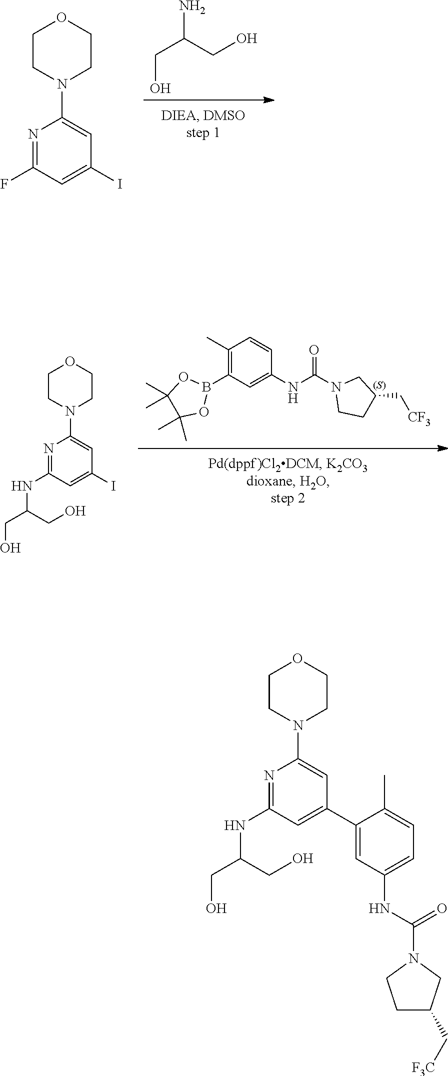

Preparation 65A: 2-((4-iodo-6-morpholinopyridin-2-yl)amino)propane-1,3-diol

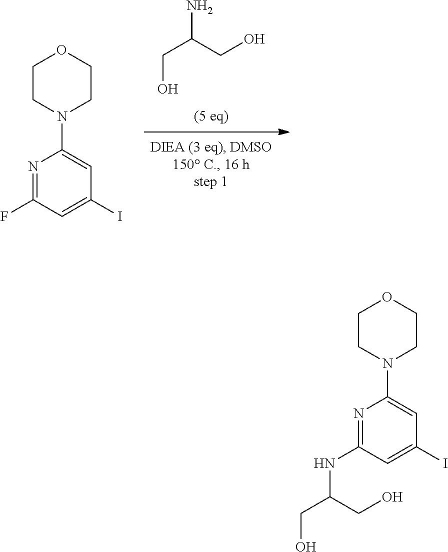 [SMILES: Fc1cc(I)cc(N2CCOCC2)n1 ; NC(CO)CO ; OCC(CO)Nc1cc(I)cc(N2CCOCC2)n1]

To a stirred mixture of 4-(6-fluoro-4-iodopyridin-2-yl)morpholine (300 mg, 0.974 mmol) and K₂CO₃ (404 mg, 2.921 mmol) in NMP (4 mL) was added 2-aminopropane-1,3-diol (443 mg, 4.869 mmol) at room temperature. The reaction mixture was stirred for 16 h at 150° C. The resulting mixture was diluted with water (50 mL). The resulting mixture was extracted with EtOAc (3×20 mL). The combined organic layers were washed with brine (3×50 mL), dried over anhydrous Na₂SO₄. After filtration, the filtrate was concentrated under reduced pressure. The residue was purified by reverse phase chromatography, eluted with 55% CH₃CN in water (with 0.5% NH₄HCO₃). The fractions containing the desired product were collected and concentrated under reduced pressure to afford 2-((4-iodo-6-morpholinopyridin-2-yl)amino)propane-1,3-diol (110 mg, 25%) as a pink solid. MS ESI calculated for C₁₂H₁₈IN₃O₃[M+H]⁺, 380.04 found 380.05. ¹H NMR (400 MHz, DMSO-d₆) δ 6.29 (d, J=1.0 Hz, 1H), 6.19 (d, J=1.0 Hz, 1H), 4.55 (t, J=5.5 Hz, 2H), 3.64 (t, J=4.9 Hz, 4H), 3.52-3.40 (m, 4H), 3.34 (d, J=4.9 Hz, 4H), 1.06 (t, J=7.0 Hz, 1H).

### Example 65: (3S)-N-(3-[2-[(1,3-dihydroxypropan-2-yl)amino]-6-(morpholin-4-yl)pyridin-4-yl]-4-methylphenyl)-3-(2,2,2-trifluoroethyl)pyrrolidine-1-carboxamide

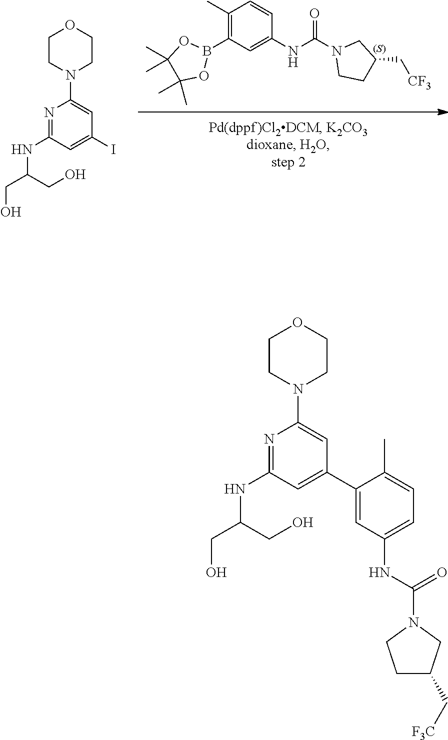

To a stirred mixture of 2-((4-iodo-6-morpholinopyridin-2-yl)amino)propane-1,3-diol (80 mg, 0.211 mmol), K₂CO₃ (87 mg, 0.633 mmol) and (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (104 mg, 0.253 mmol) in dioxane (2 mL) and H₂O (0.5 mL) was added Pd(dppf)Cl₂·CH₂Cl₂ (17 mg, 0.021 mmol) at room temperature. The reaction mixture was stirred for 2 h at 80° C. under N₂ atmosphere. The resulting mixture was diluted with water (10 mL). The resulting mixture was extracted with EtOAc (3×50 mL). The combined organic layers were washed with brine (2×50 mL), dried over anhydrous Na₂SO₄. After filtration, the filtrate was concentrated under reduced pressure. The residue was purified by silica gel column chromatography, eluted with 50% PE in EA. The fractions containing the desired product were collected and concentrated under reduced pressure. The crude was purified by reverse phase chromatography, eluted with 50% CH₃CN in water (with 10 mmol/mL NH₄HCO₃). The fractions containing the desired product were collected and concentrated under reduced pressure to afford (3S)-N-(3-[2-[(1,3-dihydroxypropan-2-yl)amino]-6-(morpholin-4-yl)pyridin-4-yl]-4-methylphenyl)-3-(2,2,2-trifluoroethyl)pyrrolidine-1-carboxamide (105 mg, 98%) as an off-white solid. MS ESI calculated for C₂₆H₃₄F₃N₅O₄[M+H]⁺, 538.26 found 538.35. ¹H NMR (400 MHz, DMSO-d₆) δ 8.11 (s, 1H), 7.41 (dd, J=8.3, 2.3 Hz, 1H), 7.34 (d, J=2.3 Hz, 1H), 7.09 (d, J=8.4 Hz, 1H), 5.86 (d, J=7.6 Hz, 1H), 5.81 (d, J=0.9 Hz, 1H), 5.76 (d, J=1.1 Hz, 1H), 4.58 (t, J=5.5 Hz, 2H), 3.82 (d, J=6.7 Hz, 1H), 3.67 (q, J=8.3, 6.4 Hz, 5H), 3.58-3.45 (m, 5H), 3.37 (d, J=9.6 Hz, 4H), 3.30 (d, J=10.0 Hz, 1H), 3.02 (t, J=9.4 Hz, 1H), 2.44 (t, J=10.7 Hz, 3H), 2.16 (s, 3H), 2.08 (d, J=3.7 Hz, 1H), 1.66 (p, J=9.9 Hz, 1H).

### Example 66: (3S)-N-[3-[2-amino-6-(morpholin-4-yl)pyridin-4-yl]-4-methylphenyl]-3-(2,2,2-trifluoroethyl)pyrrolidine-1-carboxamide

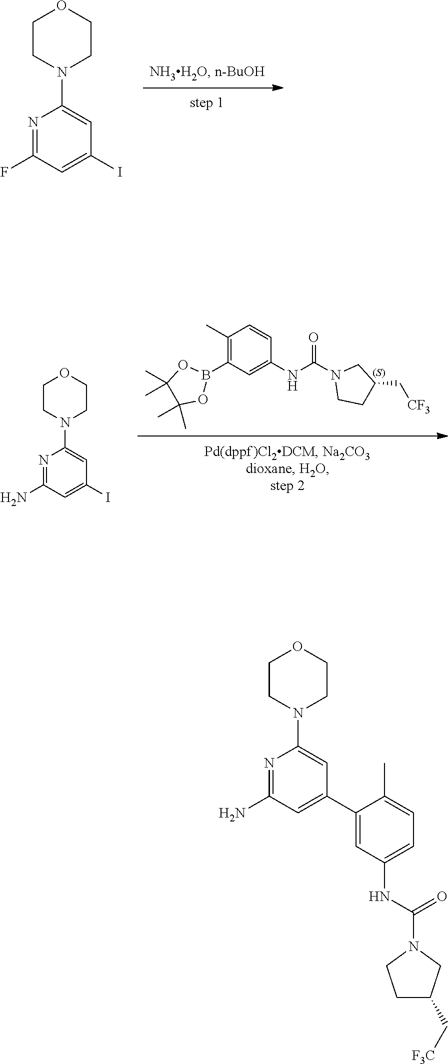

Preparation 66A: 4-iodo-6-(morpholin-4-yl)pyridin-2-amine

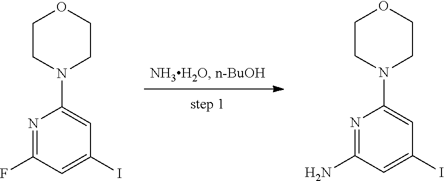 [SMILES: Fc1cc(I)cc(N2CCOCC2)n1 ; Nc1cc(I)cc(N2CCOCC2)n1]

A solution of 4-(6-fluoro-4-iodopyridin-2-yl)morpholine (2.00 g, 6.492 mmol) and NH₄OH (140 mL) in n-BuOH (20 mL) was stirred for 16 h at 120° C. The mixture was allowed to cool down to room temperature. The resulting mixture was extracted with EtOAc (3×50 mL). The combined organic layers were washed with brine (3×50 mL), dried over anhydrous Na₂SO₄. After filtration, the filtrate was concentrated under reduced pressure. The residue was purified by silica gel column chromatography, eluted with PE/EtOAc (1/1) to afford 4-iodo-6-(morpholin-4-yl)pyridin-2-amine (0.70 g, 35%) as a light yellow oil. MS ESI calculated for C₉H₁₂IN₃O [M+H]⁺, 306.00, found 305.95. ¹H NMR (300 MHz, chloroform-d) δ 6.32 (dd, J=15.3, 1.0 Hz, 2H), 4.21 (s, 2H), 3.82-3.73 (m, 4H), 3.48-3.39 (m, 4H).

### Example 66: (3S)-N-[3-[2-amino-6-(morpholin-4-yl)pyridin-4-yl]-4-methylphenyl]-3-(2,2,2-trifluoroethyl)pyrrolidine-1-carboxamide

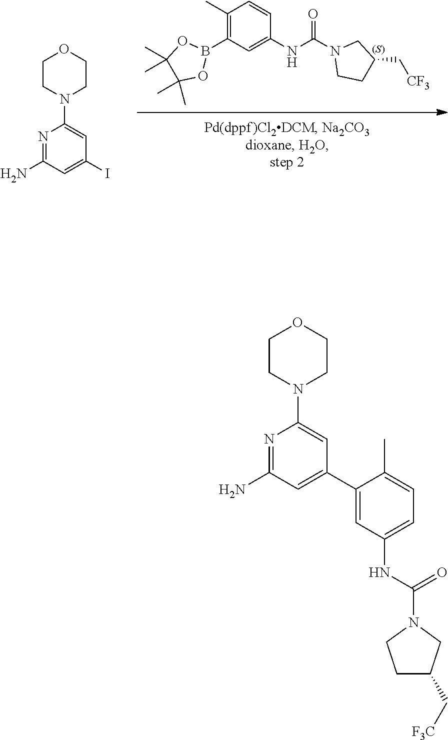

To a stirred solution of 4-iodo-6-(morpholin-4-yl)pyridin-2-amine (100 mg, 0.328 mmol) and (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (135 mg, 0.328 mmol) in 1,4-dioxane (1 mL) and H₂O (0.2 mL) were added Na₂CO₃ (104 mg, 0.983 mmol) and Pd(dppf)Cl₂·CH₂Cl₂ (27 mg, 0.033 mmol) at room temperature. The resulting mixture was stirred for 2 h at 80° C. under nitrogen atmosphere. The mixture was allowed to cool down to room temperature. The reaction was quenched by the addition of water (50 mL) at room temperature. The resulting mixture was extracted with EtOAc (3×50 mL). The combined organic layers were washed with brine (3×50 mL), dried over anhydrous Na₂SO₄. After filtration, the filtrate was concentrated under reduced pressure. The residue was purified by silica gel column chromatography, eluted with PE/EtOAc (1/1) to afford (3S)-N-[3-[2-amino-6-(morpholin-4-yl)pyridin-4-yl]-4-methylphenyl]-3-(2,2,2-trifluoroethyl)pyrrolidine-1-carboxamide (73 mg, 48%) as a light yellow solid. MS ESI calculated for C₂₃H₂₈F₃N₅O₂ [M+H]⁺, 464.22, found 464.10. ¹H NMR (400 MHz, DMSO-d₆) δ 8.11 (s, 1H), 7.42 (dd, J=8.3, 2.4 Hz, 1H), 7.34 (d, J=2.4 Hz, 1H), 7.09 (d, J=8.4 Hz, 1H), 5.77 (dd, J=17.6, 1.0 Hz, 2H), 5.64 (s, 2H), 3.71-3.63 (m, 5H), 3.55-3.49 (m, 1H), 3.41-3.35 (m, 3H), 3.32-3.26 (m, 2H), 3.02 (t, J=9.4 Hz, 1H), 2.51-2.36 (m, 3H), 2.16 (s, 3H), 2.08 (d, J=10.6 Hz, 1H), 1.67 (q, J=10.4, 10.0 Hz, 1H). ¹⁹F NMR (376 MHz, DMSO-d₆) δ-63.37 (3F).

### Example 67: (3S)-N-[4-methyl-3-[2-(methylamino)-6-(morpholin-4-yl)pyridin-4-yl]phenyl]-3-(2,2,2-trifluoroethyl)pyrrolidine-1-carboxamide

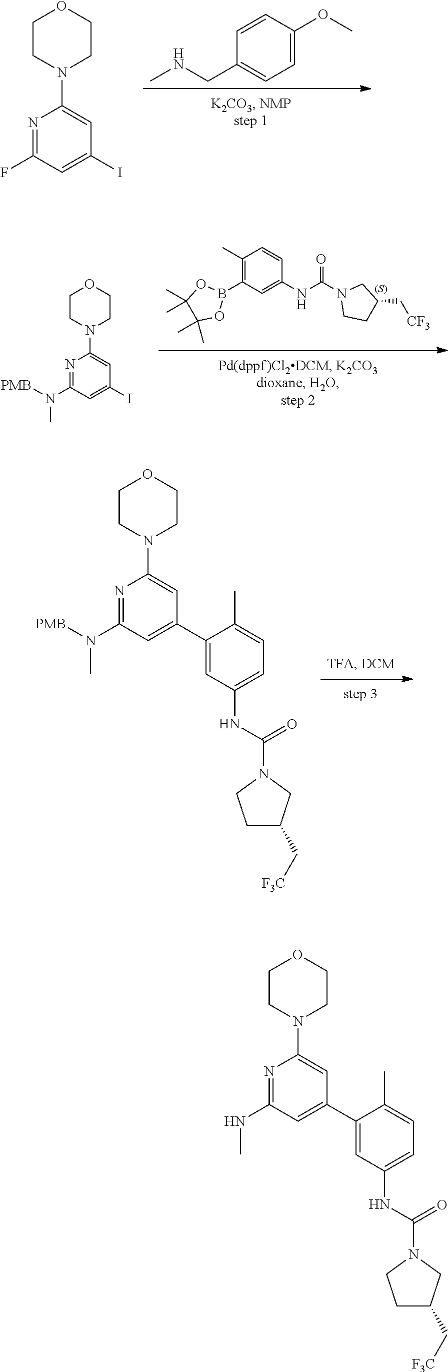

Preparation 67A: 4-iodo-N-[(4-methoxyphenyl)methyl]-N-methyl-6-(morpholin-4-yl)pyridin-2-amine

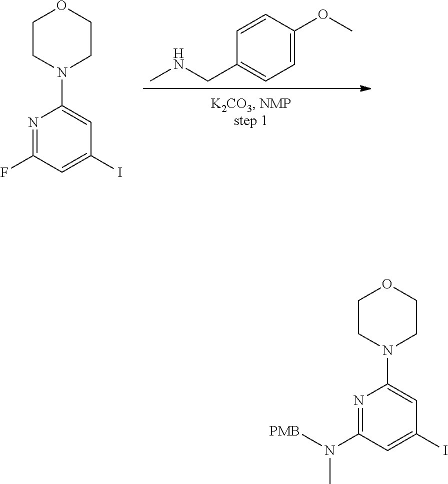

To a stirred mixture of 4-(6-fluoro-4-iodopyridin-2-yl)morpholine (320 mg, 0.974 mmol) and [(4-methoxyphenyl)methyl](methyl)amine (740 mg, 4.869 mmol) in NMP (3 mL) was added K₂CO₃(670 mg, 4.869 mmol) in portions at room temperature. The resulting mixture was stirred for 16 h at 150° C. The resulting mixture was extracted with EtOAc (3×50 mL). The combined organic layers were washed with brine (3×50 mL), dried over anhydrous Na₂SO₄. After filtration, the filtrate was concentrated under reduced pressure. The residue was purified by silica gel column chromatography, eluted with PE/EtOAc (5/1) to afford 4-iodo-N-[(4-methoxyphenyl)methyl]-N-methyl-6-(morpholin-4-yl)pyridin-2-amine (170 mg, 39%) as a yellow solid. MS ESI calculated for C₁₈H₂₂IN₃O₂[M+H]⁺, 440.08 found 440.05. ¹H NMR (300 MHz, methanol-d₄) δ 7.16 (d, J=8.5 Hz, 2H), 6.86 (d, J=8.5 Hz, 2H), 6.35 (s, 1H), 4.70 (s, 2H), 3.81 (d, J=5.0 Hz, 7H), 3.46 (t, J=4.9 Hz, 3H), 2.96 (s, 3H).

Preparation 67B: (3S)-N-[3-(2-[[(4-methoxyphenyl)methyl](methyl)amino]-6-(morpholin-4-yl)pyridin-4-yl)-4-methylphenyl]-3-(2,2,2-trifluoroethyl)pyrrolidine-1-carboxamide

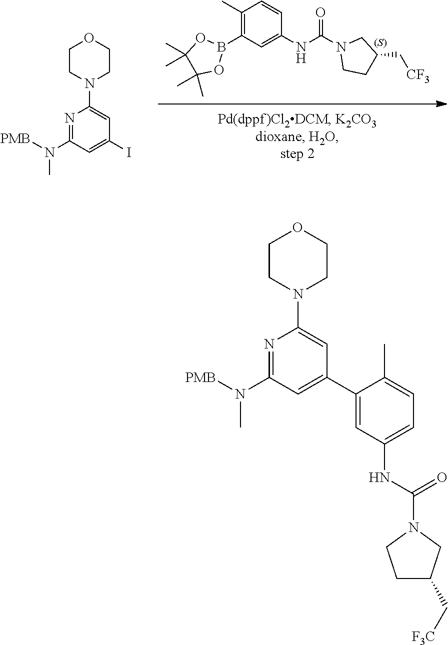

To a stirred mixture of 4-iodo-N-[(4-methoxyphenyl)methyl]-N-methyl-6-(morpholin-4-yl)pyridin-2-amine (170 mg, 0.387 mmol) and (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (160 mg, 0.387 mmol) and K₂CO₃ (160 mg, 1.161 mmol) in dioxane (1.6 mL) and H₂O (0.4 mL) was added Pd(dppf)Cl₂·CH₂Cl₂ (31 mg, 0.039 mmol) The resulting mixture was stirred for 2 h at 80° C. under nitrogen atmosphere. The resulting mixture was concentrated under reduced pressure. The residue was purified by Prep-TLC (PE/EtOAc=1/1) to afford (3S)-N-[3-(2-[[(4-methoxyphenyl)methyl](methyl)amino]-6-(morpholin-4-yl)pyridin-4-yl)-4-methylphenyl]-3-(2,2,2-trifluoroethyl)pyrrolidine-1-carboxamide (240 mg, 93%) as a light yellow solid. MS ESI calculated for C₃₂H₃₈F₃N₅O₃[M+H]⁺, 598.29 found 598.30. ¹H NMR (400 MHz, chloroform-d) δ 7.37 (d, J=8.3 Hz, 1H), 7.19 (dd, J=15.4, 8.2 Hz, 3H), 6.87 (d, J=8.5 Hz, 2H), 6.10 (s, 1H), 5.97 (s, 1H), 4.77 (s, 2H), 3.84 (d, J=19.9 Hz, 7H), 3.64 (t, J=8.9 Hz, 1H), 3.57-3.40 (m, 4H), 3.06 (s, 2H), 2.28 (t, J=9.5 Hz, 3H), 1.81-1.73 (m, 1H).

### Example 67: (3S)-N-[4-methyl-3-[2-(methylamino)-6-(morpholin-4-yl)pyridin-4-yl]phenyl]-3-(2,2,2-trifluoroethyl)pyrrolidine-1-carboxamide

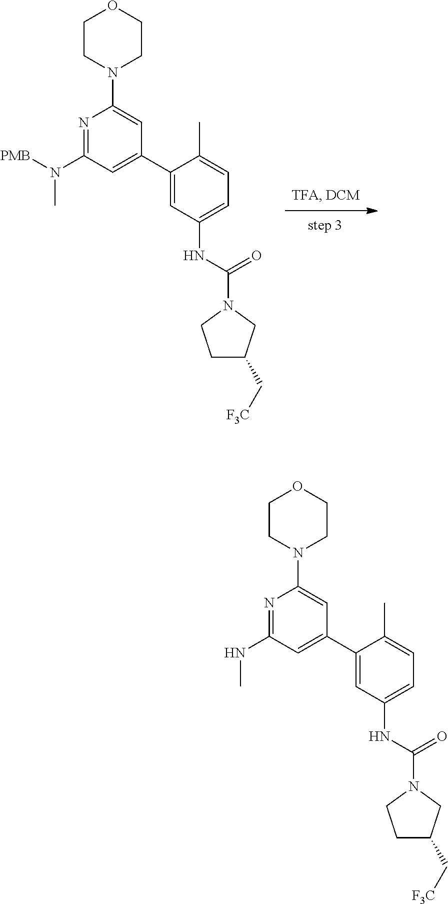

A mixture of (3S)-N-[3-(2-[[(4-methoxyphenyl)methyl](methyl)amino]-6-(morpholin-4-yl)pyridin-4-yl)-4-methylphenyl]-3-(2,2,2-trifluoroethyl)pyrrolidine-1-carboxamide (190 mg, 0.318 mmol) and TFA (1.5 mL) in DCM (1.5 mL) was stirred for 16 h at room temperature. The resulting mixture was concentrated under reduced pressure. The residue was purified by silica gel column chromatography, eluted with PE/EtOAc (1/1) to afford (3S)-N-[4-methyl-3-[2-(methylamino)-6-(morpholin-4-yl)pyridin-4-yl]phenyl]-3-(2,2,2-trifluoroethyl)pyrrolidine-1-carboxamide (90 mg, 59%) as an off-white solid. MS ESI calculated for C₂₄H₃₀F₃N₅O₂ [M+H]⁺, 478.24, found 478.05. ¹H NMR (300 MHz, methanol-d₄) δ 7.34-7.25 (m, 2H), 7.15 (d, J=8.6 Hz, 1H), 5.89 (d, J=1.3 Hz, 1H), 5.82 (d, J=1.2 Hz, 1H), 3.84-3.75 (m, 5H), 3.64 (t, J=9.8 Hz, 1H), 3.50-3.39 (m, 5H), 3.20-3.07 (m, 1H), 2.91-2.84 (m, 3H), 2.55 (s, 1H), 2.48-2.29 (m, 2H), 2.23 (s, 4H), 1.77 (p, J=10.9, 10.2 Hz, 1H). ¹⁹F NMR (376 MHz, methanol-d₄) δ-63.36 (3F).

### Example 68: (3S)-N-[3-(2-[[(2R)-1-hydroxypropan-2-yl]amino]-6-(3-oxomorpholin-4-yl)pyridin-4-yl)-4-methylphenyl]-3-(2,2,2-trifluoroethyl)pyrrolidine-1-carboxamide

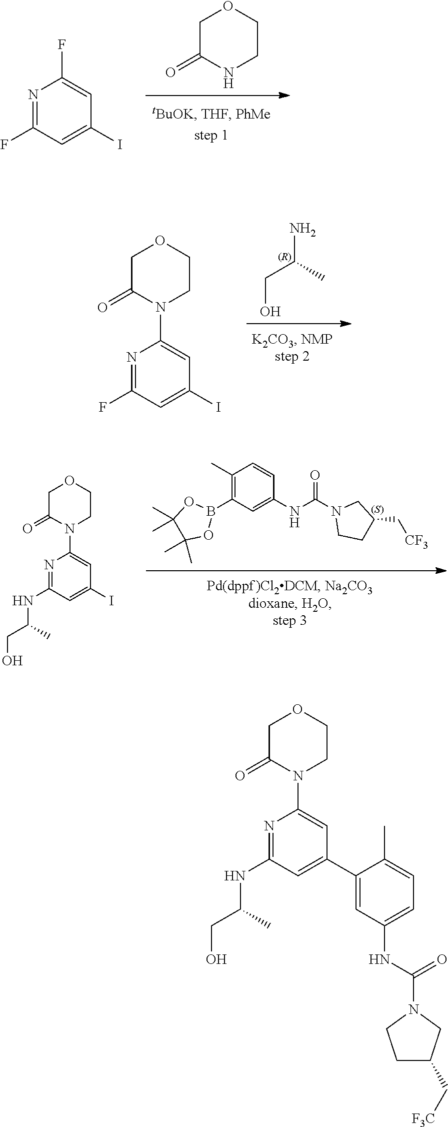

Preparation 68A: 4-(6-fluoro-4-iodopyridin-2-yl) morpholin-3-one

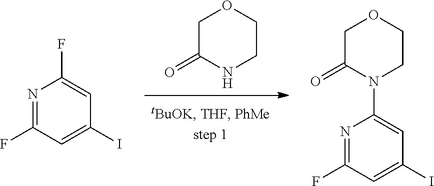 [SMILES: Fc1cc(I)cc(F)n1 ; O=C1COCCN1 ; O=C1COCCN1c1cc(I)cc(F)n1]

To a solution of morpholin-3-one (0.92 g, 9.13 mmol) in toluene (20 mL) was added t-BuOK in THF (8.3 mL, 8.30 mmol, 1 M) dropwise at 0° C. under nitrogen atmosphere. To the above mixture was added 2,6-difluoro-4-iodopyridine (2.00 g, 8.30 mmol) at room temperature. The resulting mixture was stirred for 5 h at 80° C. The reaction was quenched with water (20 mL) at 0° C. The resulting mixture was extracted with EtOAc (3×50 mL). The combined organic layers were washed with brine (3×50 mL), dried over anhydrous Na₂SO₄. After filtration, the filtrate was concentrated under reduced pressure. The residue was purified by silica gel column chromatography, eluted with hexane/EtOAc (1/1) to afford 4-(6-fluoro-4-iodopyridin-2-yl) morpholin-3-one (2.00 g, 74%) as a white solid. MS ESI calculated for C₉H₈FIN₂O₂[M+H]⁺, 322.96, found 323.00. ¹H NMR (400 MHz, chloroform-d) δ 8.63 (t, J=1.2 Hz, 1H), 7.15 (dd, J=3.3, 1.0 Hz, 1H), 4.36 (s, 2H), 4.03 (s, 4H).

Preparation 68B: 4-(6-[[(2R)-1-hydroxypropan-2-yl]amino]-4-iodopyridin-2-yl) morpholin-3-one

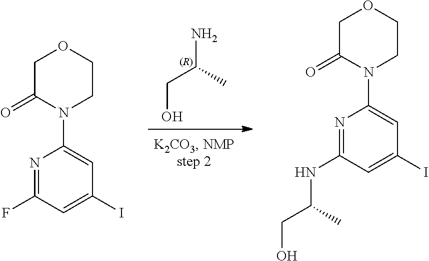

To a solution of 4-(6-fluoro-4-iodopyridin-2-yl) morpholin-3-one (1.00 g, 3.10 mmol) and (R)-(-)-2-amino-1-propanol (0.35 g, 4.66 mmol) in NMP (10 mL) was added K₂CO₃ (0.86 g, 6.21 mmol). The resulting mixture was stirred for 16 h at 100° C. The reaction was quenched by the addition of water (100 mL) at room temperature. The resulting mixture was extracted with EtOAc (3×60 mL). The combined organic layers were washed with brine (3×100 mL), dried over anhydrous Na₂SO₄. After filtration, the filtrate was concentrated under reduced pressure. The residue was purified by silica gel column chromatography, eluted with PE/EtOAc (1/1) to afford 4-(6-[[(2R)-1-hydroxypropan-2-yl]amino]-4-iodopyridin-2-yl) morpholin-3-one (0.40 g, crude) as a yellow oil. MS ESI calculated for C₁₂H₁₆IN₃O₃[M+H]⁺, 378.02; found 378.00.

### Example 68: (3S)-N-[3-(2-[[(2R)-1-hydroxypropan-2-yl]amino]-6-(3-oxomorpholin-4-yl)pyridin-4-yl)-4-methylphenyl]-3-(2,2,2-trifluoroethyl)pyrrolidine-1-carboxamide

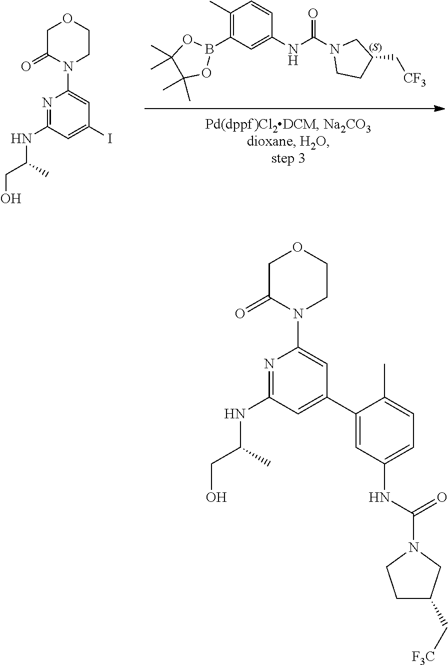

A mixture of 4-(6-[[(2R)-1-hydroxypropan-2-yl]amino]-4-iodopyridin-2-yl) morpholin-3-one (150 mg, 0.398 mmol), (3S)-N-[4-methyl-3-(4,4,5,5-tetramethyl-1,3,2-dioxaborolan-2-yl)phenyl]-3-(2,2,2-trifluoroethyl) pyrrolidine-1-carboxamide (164 mg, 0.398 mmol), Pd(dppf)Cl₂·CH₂Cl₂ (32 mg, 0.040 mmol), Na₂CO₃ (126 mg, 1.193 mmol), dioxane (2 mL) and H₂O (0.2 mL). The mixture was stirred for 2 h at 80° C. under nitrogen atmosphere. The reaction mixture was concentrated under reduced pressure. The residue was purified by reverse phase chromatography with the following conditions: (Column: Spherical C18, 20-40 um, 40 g; Mobile Phase A: Water (10 mmol/mL NH₄HCO₃), Mobile Phase B: CH₃CN; Flow rate: 40 mL/min; 30%-60% within 20 min). The collected fractions were combined and concentrated under reduced pressure to afford (3S)-N-[3-(2-[[(2R)-1-hydroxypropan-2-yl]amino]-6-(3-oxomorpholin-4-yl)pyridin-4-yl)-4-methylphenyl]-3-(2,2,2-trifluoroethyl)pyrrolidine-1-carboxamide (45 mg, 21%) as a white solid. MS ESI calculated for C₂₆H₃₂F₃N₅O₄[M+H]⁺, 536.25 found 536.24. ¹H NMR (300 MHz, DMSO-d₆) δ 8.17 (s, 1H), 7.49-7.33 (m, 2H), 7.17-7.06 (m, 2H), 6.44 (d, J=7.6 Hz, 1H), 6.26 (d, J=1.2 Hz, 1H), 4.69 (t, J=5.6 Hz, 1H), 4.21 (s, 2H), 3.97 (s, 5H), 3.68 (dd, J=10.3, 6.7 Hz, 1H), 3.59-3.46 (m, 2H), 3.32-3.25 (m, 2H), 3.03 (t, J=9.3 Hz, 1H), 2.45 (q, J=9.5, 7.6 Hz, 3H), 2.18 (s, 4H), 1.73-1.56 (m, 1H), 1.16 (d, J=6.5 Hz, 3H). ¹⁹F NMR (276 MHz, DMSO-d₆)δ-63.37 (3F).

### Example 69: (3S)-N-[3-[2-acetamido-6-(morpholin-4-yl)pyridin-4-yl]-4-methylphenyl]-3-(2,2,2-trifluoroethyl)pyrrolidine-1-carboxamide

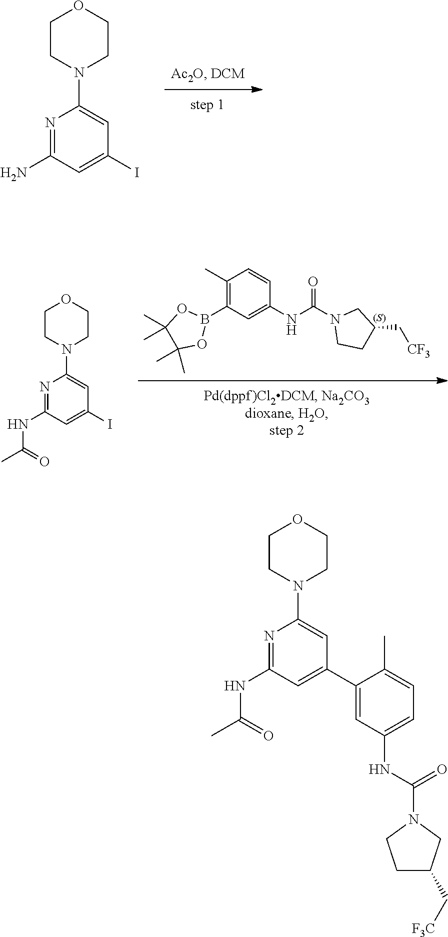

Preparation 69A: N-[4-iodo-6-(morpholin-4-yl)pyridin-2-yl]acetamide

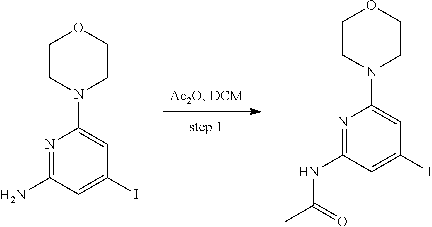 [SMILES: Nc1cc(I)cc(N2CCOCC2)n1 ; CC(=O)Nc1cc(I)cc(N2CCOCC2)n1]

To a stirred solution of 4-iodo-6-(morpholin-4-yl)pyridin-2-amine (200 mg, 0.655 mmol) in DCM (2 mL) was added 4-iodo-6-(morpholin-4-yl)pyridin-2-amine (200 mg, 0.655 mmol) at room temperature. The resulting mixture was stirred for 2 h at room temperature. The reaction was quenched by the addition of MeOH (1 mL) at room temperature. The resulting mixture was concentrated under reduced pressure. The residue was purified by silica gel column chromatography, eluted with PE/EtOAc (1/1 to 0/1) to afford N-[4-iodo-6-(morpholin-4-yl)pyridin-2-yl]acetamide (210 mg, 92%) as a white solid. MS ESI calculated for C₁₁H₁₄IN₃O₂ [M+H]⁺, 348.01, found 348.00. ¹H NMR (300 MHz, chloroform-d) δ 7.93 (s, 1H), 7.53 (s, 1H), 6.75 (d, J=1.1 Hz, 1H), 3.83-3.73 (m, 4H), 3.48-3.39 (m, 4H), 2.18 (s, 3H).

### Example 69: (3S)-N-[3-[2-acetamido-6-(morpholin-4-yl)pyridin-4-yl]-4-methylphenyl]-3-(2,2,2-trifluoroethyl)pyrrolidine-1-carboxamide

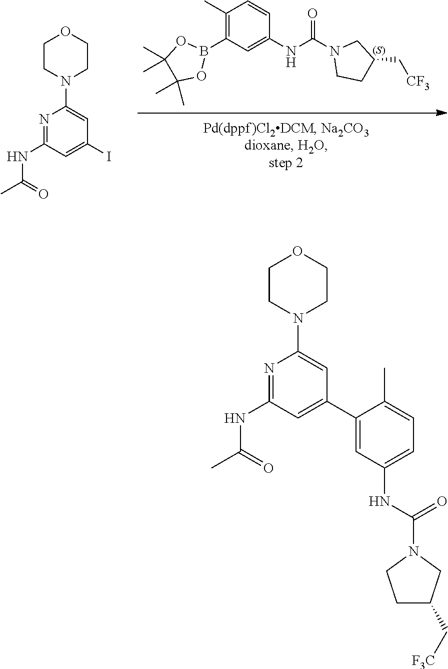

To a stirred solution of N-[4-iodo-6-(morpholin-4-yl)pyridin-2-yl]acetamide (200 mg, 0.576 mmol) and (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (237 mg, 0.576 mmol), 1,4-dioxane (2 mL) and H₂O (0.4 mL) were added Na₂CO₃ (183 mg, 1.728 mmol) and Pd(dppf)Cl₂·CH₂Cl₂(47 mg, 0.058 mmol). The resulting mixture was stirred for 2 h at 80° C. under nitrogen atmosphere. The mixture was allowed to cool down to room temperature. The reaction was quenched by the addition of water (50 mL) at room temperature. The resulting mixture was extracted with EtOAc (3×50 mL). The combined organic layers were washed with brine (3×50 mL), dried over anhydrous Na₂SO₄. After filtration, the filtrate was concentrated under reduced pressure. The residue was purified by silica gel column chromatography, eluted with PE/EtOAc (1/2) to afford (3S)-N-[3-[2-acetamido-6-(morpholin-4-yl)pyridin-4-yl]-4-methylphenyl]-3-(2,2,2-trifluoroethyl)pyrrolidine-1-carboxamide(89 mg, 31%) as a light pink solid. MS ESI calculated for C₂₅H₃₀F₃N₅O₃ [M+H]⁺, 506.23, found 506.10. ¹H NMR (400 MHz, DMSO-d₆) δ 10.04 (s, 1H), 8.16 (s, 1H), 7.47 (dd, J=8.3, 2.4 Hz, 1H), 7.37 (d, J=2.4 Hz, 2H), 7.14 (d, J=8.4 Hz, 1H), 6.40 (d, J=1.1 Hz, 1H), 3.73-3.63 (m, 5H), 3.58-3.44 (m, 5H), 3.35-3.25 (m, 1H), 3.02 (t, J=9.5 Hz, 1H), 2.51-2.36 (m, 3H), 2.16 (s, 3H), 2.08 (s, 4H), 1.72-1.60 (m, 1H). ¹⁹F NMR (376 MHz, DMSO-d₆) δ-66.37 (3F).

### Example 70: (S)-N-(3-(3-fluoro-2-(((R)-1-hydroxypropan-2-yl)amino)-6-morpholinopyridin-4-yl)-4-methylphenyl)-3-(2,2,2-trifluoroethyl)pyrrolidine-1-carboxamide

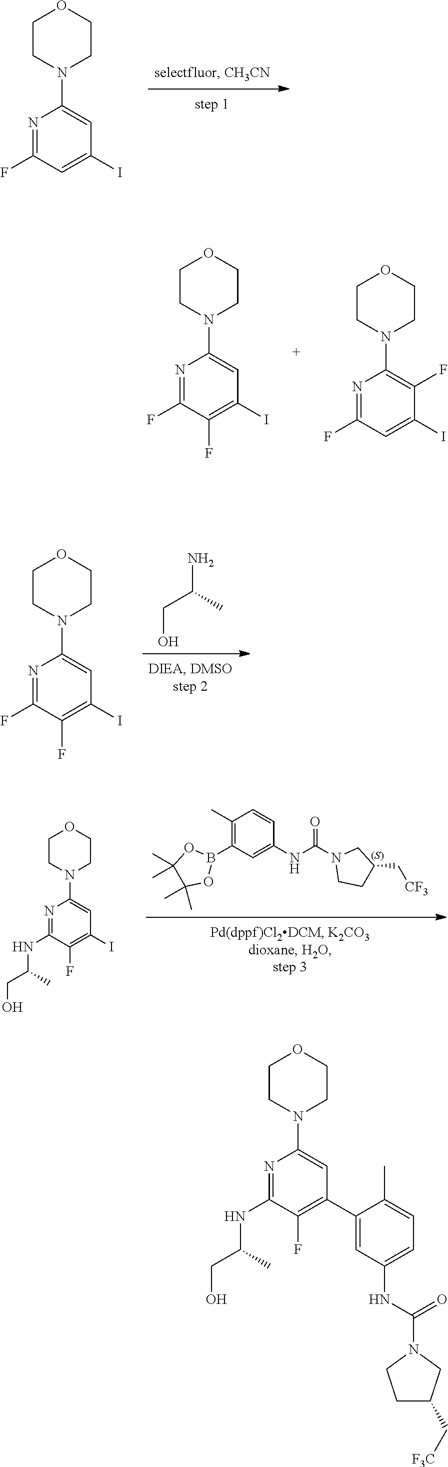

Preparation 70A: 4-(5,6-difluoro-4-iodopyridin-2-yl)morpholine and 4-(3,6-difluoro-4-iodopyridin-2-yl)morpholine

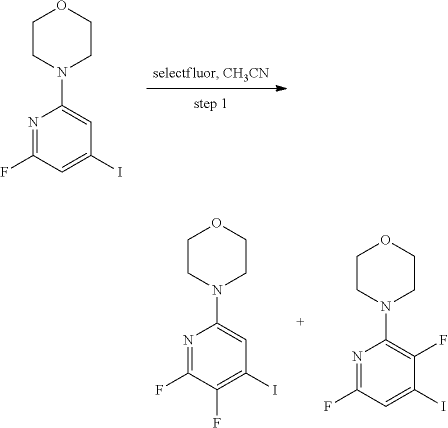 [SMILES: Fc1cc(I)cc(N2CCOCC2)n1 ; Fc1nc(N2CCOCC2)cc(I)c1F ; Fc1cc(I)c(F)c(N2CCOCC2)n1]

To a solution of 4-(6-fluoro-4-iodopyridin-2-yl)morpholine (6.00 g, 19.475 mmol) in MeCN (240 mL) was added selectfluor (6.9 g, 19.475 mmol) in portions at room atmosphere. The mixture was stirred for 16 h at room atmosphere. The resulting mixture was quenched with water (300 mL), and extracted with EtOAc (3×500 mL). The combined organic layers were washed with brine (2×150 mL), dried over anhydrous Na₂SO₄ and concentrated under redunced pressure. The residue was purified by silica gel column chromatography, eluted with EtOAc in PE (0˜90%) to afford 4-(5,6-difluoro-4-iodopyridin-2-yl)morpholine (700 mg, 11%) as a white solid. MS ESI calculated for C₉H₉F₂IN₂O [M+H]⁺, 326.97, found 326.90. ¹H NMR (400 MHz, Chloroform-d) δ 6.75 (t, J=1.8 Hz, 1H), 3.84-3.76 (m, 4H), 3.43 (dd, J=5.8, 4.1 Hz, 4H). Also afforded 4-(3,6-difluoro-4-iodopyridin-2-yl)morpholine (3.2 g, 50%) as a white solid. MS ESI calculated for C₉H₉F₂IN₂O [M+H]⁺, 326.97, found 326.95. ¹H NMR (400 MHz, Chloroform-d) δ 6.68 (dd, J=4.2, 2.3 Hz, 1H), 3.82-3.80 (m, 4H), 3.59-3.56 (m, 4H).

Preparation 70B: (R)-2-((3-fluoro-4-iodo-6-morpholinopyridin-2-yl)amino)propan-1-ol

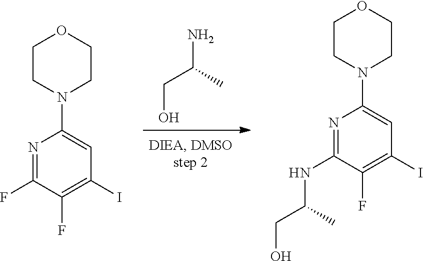

A mixture of 4-(5,6-difluoro-4-iodopyridin-2-yl)morpholine (300 mg, 0.920 mmol), (R)-(−)-2-amino-1-propanol (138 mg, 1.840 mmol) and K₂CO₃ (254 mg, 1.840 mmol) in NMP (5 mL) at room temperture, the mixture was stirred at 70° C. for 16 h. The mixture was allowed to cool down to room temperature. The reaction was quenched with water (50 mL) at room temperature. The resulting mixture was extracted with EtOAc (50 mL). The combined organic layers were washed with brine (3×50 mL), dried over anhydrous Na₂SO₄. After filtration, the filtrate was concentrated under reduced pressure. The residue was purified by Prep-TLC (PE/EtOAc/EtOH=12/3/1) to afford (2R)-2-[[3-fluoro-4-iodo-6-(morpholin-4-yl)pyridin-2-yl]amino]propan-1-ol (224 mg, 64%) as a yellow oil. MS ESI calculated for C₁₂H₁₇FIN₃O₂[M+H]⁺, 382.03, found 381.95. ¹H NMR (400 MHz, chloroform-d) δ 6.17 (d, J=2.4 Hz, 1H), 4.28-4.20 (m, 1H), 3.84-3.80 (m, 4H), 3.80-3.75 (m, 1H), 3.62 (dd, J=10.7, 7.0 Hz, 1H), 3.37 (dd, J=5.9, 3.9 Hz, 4H), 1.31-1.28 (m, 3H).

### Example 70: (S)-N-(3-(3-fluoro-2-(((R)-1-hydroxypropan-2-yl)amino)-6-morpholinopyridin-4-yl)-4-methylphenyl)-3-(2,2,2-trifluoroethyl)pyrrolidine-1-carboxamide

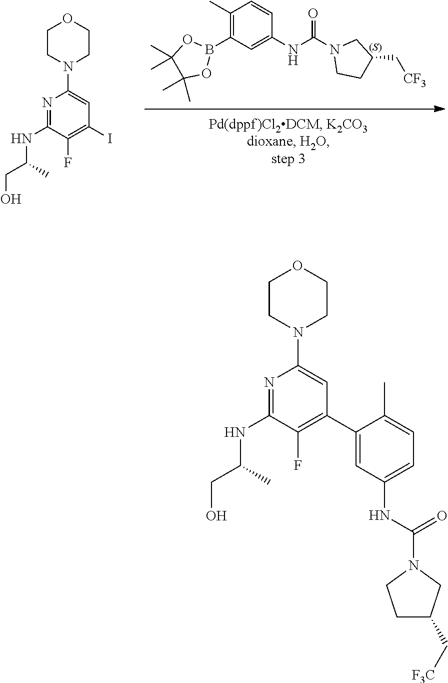

A mixture of (2R)-2-[[3-fluoro-4-iodo-6-(morpholin-4-yl)pyridin-2-yl]amino]propan-1-ol (200 mg, 0.525 mmol), (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (238 mg, 0.577 mmol), Pd(dppf)Cl₂. CH₂Cl₂ (43 mg, 0.052 mmol) and K₂CO₃ (217 mg, 1.574 mmol) in dioxane (8 mL) and H₂O (2 mL) stirred at 80° C. for 2 h. The mixture was allowed to cool down to room temperature. The resulting mixture was concentrated under reduced pressure. The mixture was purified by Prep-TLC (PE/EtOAc/EtOH=4:3:1) followed by reverse flash chromatography with the following Column: C18 Column 120 g; Mobile Phase A: water (10 mmoL NH₄HCO₃), Mobile Phase B: CH₃CN; Flow rate: 60 mL/min; Gradient: 40% to 65% B in 30 min; 254/220 nm. The fractions containing the desired product was collected at 55% B and concentrated under reduced pressure to afford (3S)-N-[3-(3-fluoro-2-[[(2R)-1-hydroxypropan-2-yl]amino]-6-(morpholin-4-yl)pyridin-4-yl)-4-methylphenyl]-3-(2,2,2-trifluoroethyl)pyrrolidine-1-carboxamide (128 mg, 45%) as a white solid. MS ESI calculated for C₂₆H₃₃F₄N₅O₃[M+H]⁺, 540.58, found 540.30. ¹H NMR (400 MHz, DMSO-d₆) δ 8.15 (s, 1H), 7.45 (dd, J=8.3, 2.4 Hz, 1H), 7.37 (d, J=2.3 Hz, 1H), 7.14 (d, J=8.4 Hz, 1H), 5.89 (dd, J=7.7, 1.8 Hz, 1H), 5.63 (d, J=3.0 Hz, 1H), 4.67 (t, J=5.7 Hz, 1H), 4.07 (p, J=6.4 Hz, 1H), 3.71-3.66 (m, 4H), 3.64 (s, 1H), 3.57-3.46 (m, 2H), 3.41-3.35 (m, 1H), 3.32-3.23 (m, 4H), 3.02 (t, J=9.4 Hz, 1H), 2.50-2.35 (m, 4H), 2.09 (s, 4H), 1.66 (p, J=9.9 Hz, 1H), 1.16 (d, J=6.6 Hz, 3H). ¹⁹F NMR (376 MHz, DMSO-d₆) δ-63.37 (3F), −158.76 (1F).

### Example 71: (3S)-N-[3-(3-fluoro-6-[[(2R)-1-hydroxypropan-2-yl]amino]-2-(morpholin-4-yl)pyridin-4-yl)-4-methylphenyl]-3-(2,2,2-trifluoroethyl)pyrrolidine-1-carboxamide

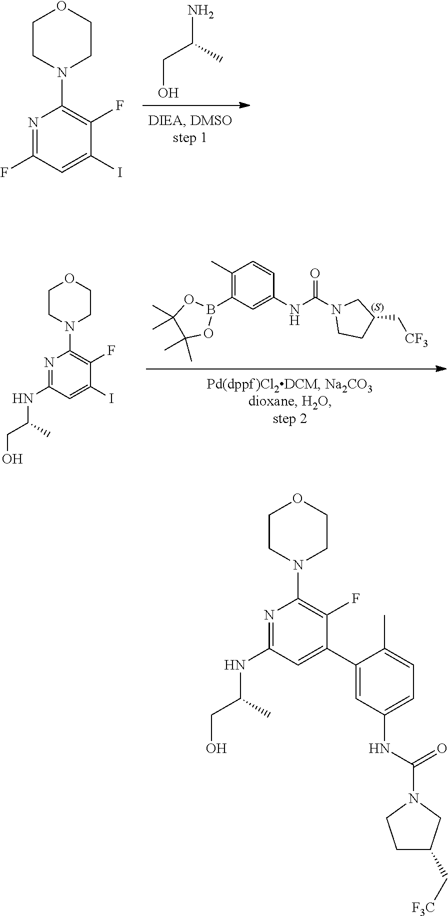

Preparation 71A: (2R)-2-[[5-fluoro-4-iodo-6-(morpholin-4-yl)pyridin-2-yl]amino]propan-1-ol

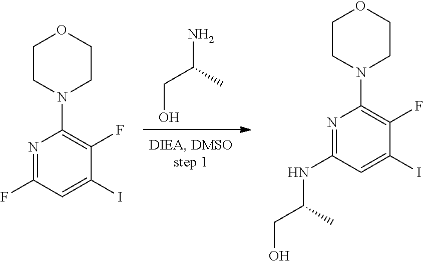

To a stirred solution of 4-(3,6-difluoro-4-iodopyridin-2-yl)morpholine (900 mg, 2.760 mmol) and (R)-(−)-2-amino-1-propanol (829 mg, 11.040 mmol) in NMP (15 mL) were added K₂CO₃ (763 mg, 5.520 mmol) at room temperature. The resulting mixture was stirred for 16 h at 150° C. The mixture was allowed to cool down to room temperature. The resulting mixture was extracted with EtOAc (3×50 mL). The combined organic layers were washed with water (3×100 mL), dried over anhydrous Na₂SO₄. After filtration, the filtrate was concentrated under reduced pressure. The residue was purified by reverse phase flash chromatography with the following conditions: Column: Spherical C18, 20-40 um, 330 g; Mobile Phase A: water (10 mM NH₄HCO₃); Mobile Phase B: acetonitrile; Flow rate: 80 mL/min; Gradient: 50%-65% B in 20 min; Detector: 254 nm. The fractions containing desired product were collected at 55% B and concentrated under reduced pressure to afford (2R)-2-[[5-fluoro-4-iodo-6-(morpholin-4-yl)pyridin-2-yl]amino]propan-1-ol (175 mg, 16%) as a light yellow solid. MS ESI calculated for C₁₂H₁₆FIN₂O₃[M+H]⁺, 382.04; found 381.95. ¹H NMR (300 MHz, chloroform-d) δ 6.27 (d, 1H), 4.11 (s, 1H), 3.90 (s, 1H), 3.85-3.75 (m, 4H), 3.54 (m, 1H), 3.47-3.39 (m, 3H), 1.21 (d, 3H). ¹⁹F NMR (376 MHz, chloroform-d) 6-127.54.

### Example 71: (3S)-N-[3-(3-fluoro-6-[[(2R)-1-hydroxypropan-2-yl]amino]-2-(morpholin-4-yl)pyridin-4-yl)-4-methylphenyl]-3-(2,2,2-trifluoroethyl)pyrrolidine-1-carboxamide

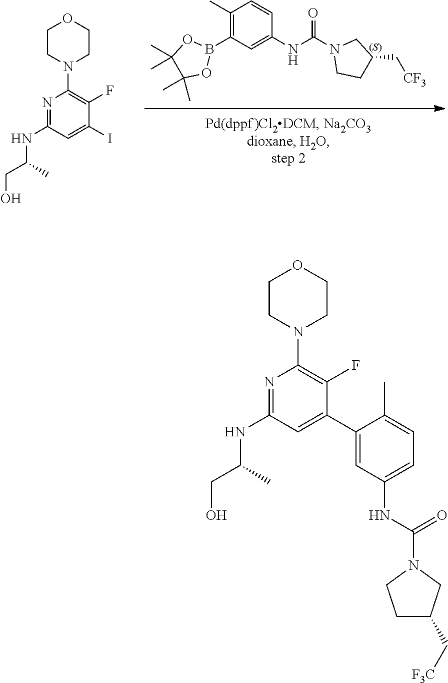

To a stirred mixture of (2R)-2-[[5-fluoro-4-iodo-6-(morpholin-4-yl)pyridin-2-yl]amino]propan-1-ol (230 mg, 0.603 mmol) and (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (298 mg, 0.724 mmol), Na₂CO₃ (191 mg, 1.810 mmol) in dioxane (4 mL), H₂O (1 mL) was added Pd(dppf)Cl₂·CH₂Cl₂ (49 mg, 0.060 mmol). The resulting mixture was stirred for 16 h at 100° C. under nitrogen atmosphere. The resulting mixture was quenched with water (20 mL), and extracted with EtOAc (3×100 mL). The combined organic layers were washed with brine (3×50 mL), dried over anhydrous Na₂SO₄ and concentrated under reduced pressure. The residue was purified by silica gel column chromatography, eluted with 0% to 50% EtOAc in PE to afford (3S)-N-[3-(3-fluoro-6-[[(2R)-1-hydroxypropan-2-yl]amino]-2-(morpholin-4-yl)pyridin-4-yl)-4-methylphenyl]-3-(2,2,2-trifluoroethyl)pyrrolidine-1-carboxamide (84 mg, 25%) as a yellow solid. MS ESI calculated for C₂₆H₃₃F₄N₅O₃[M+H]⁺, 540.26; found 540.25. ¹H NMR (300 MHz, DMSO-d₆) δ 8.15 (s, 1H), 7.49-7.34 (m, 2H), 7.13 (m, 1H), 5.99 (m, 1H), 5.78 (m, 1H), 4.64 (m, 1H), 3.99-3.59 (m, 7H), 3.58-3.45 (m, 2H), 3.32-3.22 (m, 5H), 3.03 (m, 1H), 2.44 (m, 3H), 2.08 (s, 4H), 1.66 (m, 1H), 1.12 (m, 3H). ¹⁹F NMR (282 MHz, DMSO-d₆) δ-63.36 (3F), −151.86 (1F).

### Example 72: (S)-N-(3-(2-(trans-3-hydroxy-2-methylazetidin-1-yl)-6-morpholinopyridin-4-yl)-4-methylphenyl)-3-(2,2,2-trifluoroethyl)pyrrolidine-1-carboxamide

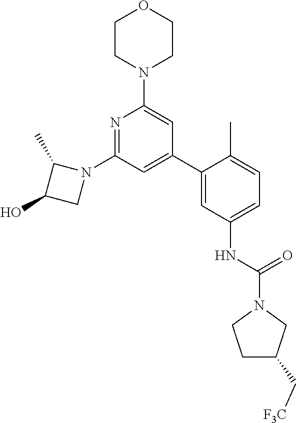 [SMILES: Cc1ccc(NC(=O)N2CC[C@@H](CC(F)(F)F)C2)cc1-c1cc(N2CCOCC2)nc(N2C[C@@H](O)[C@@H]2C)c1]

### Example 73: (S)-N-(3-(2-(cis-3-hydroxy-2-methylazetidin-1-yl)-6-morpholinopyridin-4-yl)-4-methylphenyl)-3-(2,2,2-trifluoroethyl)pyrrolidine-1-carboxamide

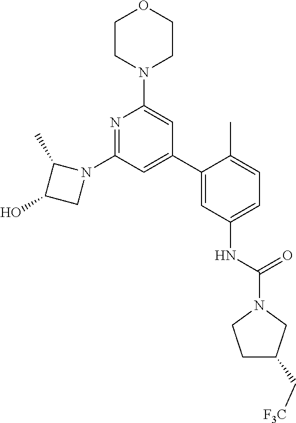 [SMILES: Cc1ccc(NC(=O)N2CC[C@@H](CC(F)(F)F)C2)cc1-c1cc(N2CCOCC2)nc(N2C[C@H](O)[C@@H]2C)c1]

### Synthetic Scheme

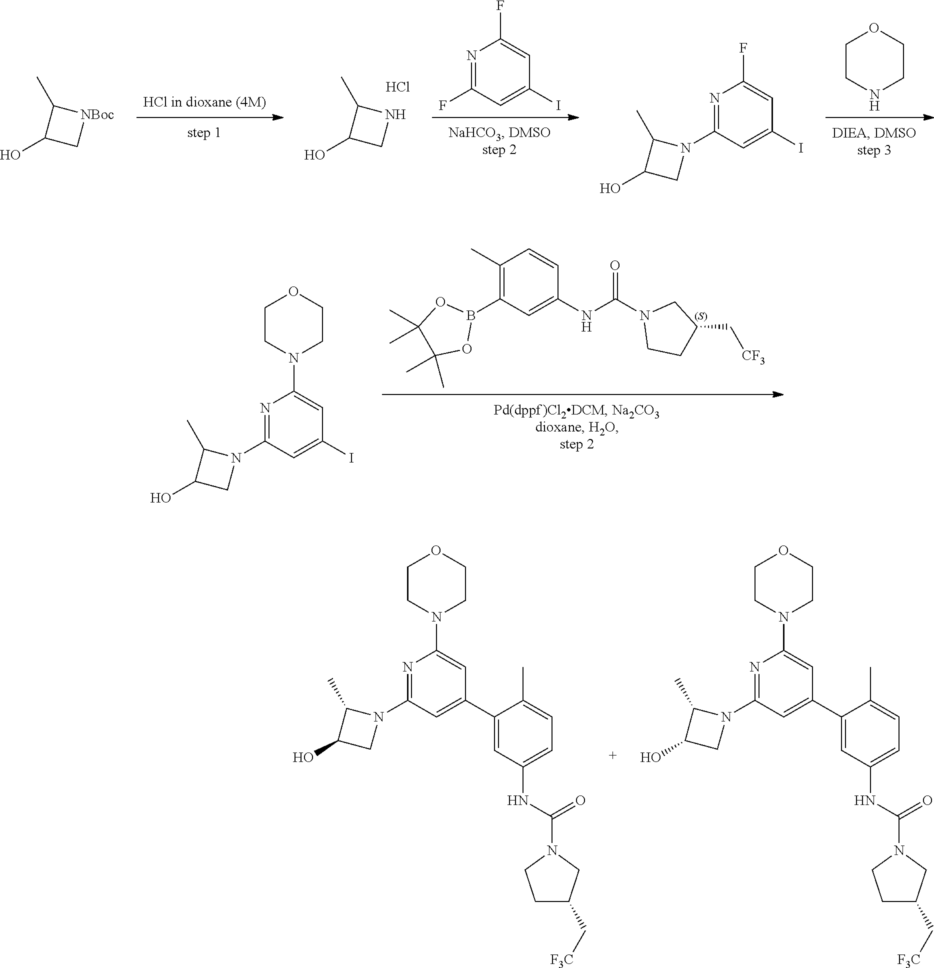

Preparation 72A: 2-methylazetidin-3-ol hydrochloride

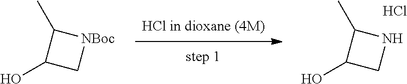

To a stirred solution of tert-butyl 3-hydroxy-2-methylazetidine-1-carboxylate (450 mg, 2.403 mmol) in 1,4-dioxane (4.5 mL) was added HCl (gas) in 1,4-dioxane (4.5 mL) dropwise at room temperature. The resulting solution was stirred for 16 h at room temperature. The reaction mixture was concentrated under reduced pressure to afford 2-methylazetidin-3-ol hydrochloride (400 mg, crude) as yellow oil. The crude product was used directly for the next step without any other purification.

Preparation 72B: 1-(6-fluoro-4-iodopyridin-2-yl)-2-methylazetidin-3-ol

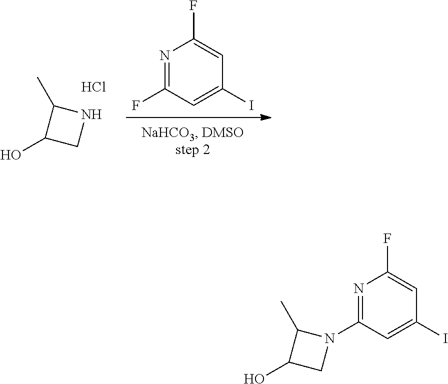

A mixture of 2,6-difluoro-4-iodopyridine (690 mg, 2.863 mmol), 2-methylazetidin-3-ol hydrochloride (389 mg, 3.150 mmol) and NaHCO₃ (721 mg, 8.590 mmol) in DMSO (7 mL) was stirred for 3 h at 100° C. The resulting mixture was cooled down to room temperature and diluted with water (100 mL) and extracted with EtOAc (3×50 mL). The combined organic layer was washed with brine (3×100 mL), dried over anhydrous Na₂SO₄. After filtration, the filtrate was concentrated under reduced pressure. The residue was purified by silica gel column chromatography, eluted with EtOAc in PE (40% to 50%) to afford 1-(6-fluoro-4-iodopyridin-2-yl)-2-methylazetidin-3-ol (270 mg, 33%) (mixture of cis and trans) as off-white semi-solid. MS ESI calculated for C₉H₁₀FIN₂O [M+H]⁺, 308.98, found 308.85. ¹H NMR (400 MHz, DMSO-d₆) δ 6.93-6.68 (m, 0.5H), 6.62 (ddt, J=4.2, 2.6, 1.4 Hz, 1H), 6.57 (t, J=1.4 Hz, 0.5H), 5.59 (dd, J=57.6, 6.2 Hz, 1H), 4.69-4.35 (m, 2H), 4.18-3.89 (m, 1H), 3.79-3.42 (m, 1H), 1.48-1.23 (m, 3H).

Preparation 72C: 1-[4-iodo-6-(morpholin-4-yl)pyridin-2-yl]-2-methylazetidin-3-ol

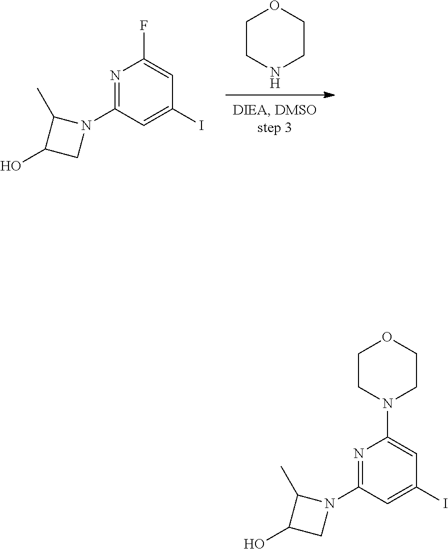 [SMILES: CC1C(O)CN1c1cc(I)cc(F)n1 ; C1COCCN1 ; CC1C(O)CN1c1cc(I)cc(N2CCOCC2)n1]

A solution of 1-(6-fluoro-4-iodopyridin-2-yl)-2-methylazetidin-3-ol (275 mg, 0.893 mmol), morpholine (85 mg, 0.982 mmol) and DIEA (230 mg, 1.785 mmol) in DMSO (3 mL) was stirred for 16 h at 100° C. The resulting mixture was cooled down to room temperature. The resulting solution was quenched with water (50 mL) and extracted with EtOAc (3×30 mL). The combined organic layer was washed with brine (1×50 mL), dried over anhydrous Na₂SO₄. After filtration, the filtrate was concentrated under reduced pressure. The residue was purified by silica gel column chromatography, eluted with EtOAc in PE (48% to 60%) to afford 1-[4-iodo-6-(morpholin-4-yl)pyridin-2-yl]-2-methylazetidin-3-ol (170 mg, 36%) (mixture of cis and trans) as a light yellow solid. MS ESI calculated for C₁₃H₁₈IN₃O₂ [M+H]⁺, 376.04, found 375.95. ¹H NMR (400 MHz, chloroform-d) δ 6.34 (dd, J=17.3, 1.2 Hz, 1H), 6.09 (dd, J=17.7, 1.0 Hz, 1H), 4.62 (t, J=5.4 Hz, 1H), 4.53-4.34 (m, 1H), 4.25-4.03 (m, 1H), 3.80 (q, J=5.2 Hz, 5H), 3.54-3.36 (m, 4H), 1.49 (dd, J=29.7, 6.5 Hz, 3H).

### Example 72: (S)-N-(3-(2-(trans-3-hydroxy-2-methylazetidin-1-yl)-6-morpholinopyridin-4-yl)-4-methylphenyl)-3-(2,2,2-trifluoroethyl)pyrrolidine-1-carboxamide

### Example 73: (S)-N-(3-(2-(cis-3-hydroxy-2-methylazetidin-1-yl)-6-morpholinopyridin-4-yl)-4-methylphenyl)-3-(2,2,2-trifluoroethyl)pyrrolidine-1-carboxamide

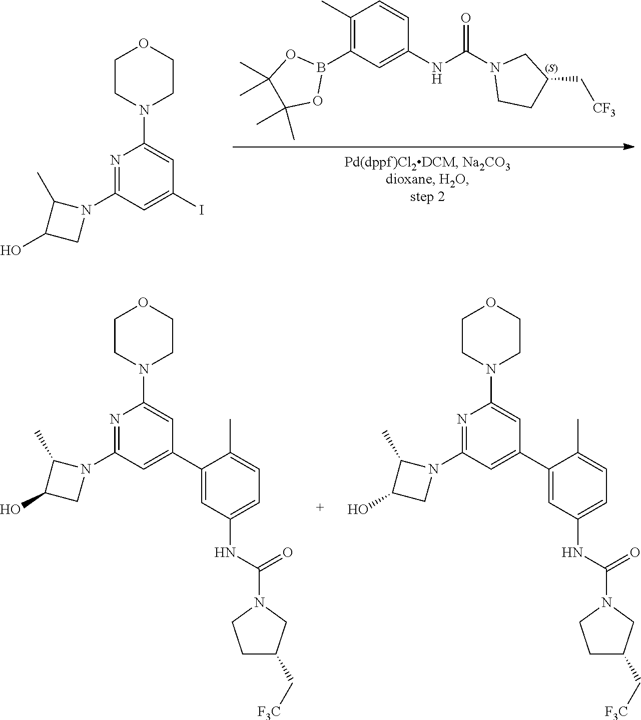

A mixture of 1-[4-iodo-6-(morpholin-4-yl)pyridin-2-yl]-2-methylazetidin-3-ol (170 mg, 0.453 mmol), (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (205 mg, 0.498 mmol), Pd(dppf)Cl₂·CH₂Cl₂ (37 mg, 0.045 mmol), Na₂CO₃ (144 mg, 1.359 mmol), 1,4-dioxane (4 mL) and H₂O (1 mL) was stirred for 16 h at 60° C. The reaction mixture was cooled to room temperature and concentrated under reduced pressure. The residue was purified by silica gel column chromatography, eluted with PE/EA/EtOH (12/3/1). The crude product was purified by prep-HPLC with the following conditions: Column: Prep C₁₈ OBD Column, 19×150 mm Sum 10 nm; Mobile Phase A: water (0.5% TFA), Mobile Phase B: acetonitrile; Flow rate:20 mL/min; Gradient: 35%-50% B in 4.3 min; to afford (S)-N-(3-(2-(trans-3-hydroxy-2-methylazetidin-1-yl)-6-morpholinopyridin-4-yl)-4-methylphenyl)-3-(2,2,2-trifluoroethyl)pyrrolidine-1-carboxamide (73 mg, 30%) as a light yellow solid. MS ESI calculated for C₂₇H₃4F₃N₅O₃ [M+H]⁺, 534.26, found 534.25. ¹H NMR (400 MHz, DMSO-d₆) δ 8.12 (s, 1H), 7.43-7.41 (m, 1H), 7.32-7.31 (m, 1H), 7.11-7.09 (m, 1H), 5.90 (s, 1H), 5.54 (s, 1H), 5.38-5.37 (m, 1H), 4.47-4.45 (m, 1H), 4.32-4.29 (m, 1H), 4.00-3.96 (m, 1H), 3.69-3.63 (m, 6H), 3.52-3.50 (m, 1H), 3.40-3.30 (m, 5H), 3.04-2.99 (m, 1H), 2.52-2.41 (m, 3H), 2.15-2.08 (m, 4H), 1.66-1.65 (m, 1H), 1.32 (d, J=6.8 Hz, 3H). ¹⁹F NMR (376 MHz, DMSO-d₆) δ-63.36 (3F).

Also afforded (S)-N-(3-(2-(cis-3-hydroxy-2-methylazetidin-1-yl)-6-morpholinopyridin-4-yl)-4-methylphenyl)-3-(2,2,2-trifluoroethyl)pyrrolidine-1-carboxamide (24 mg, 10%) as a light yellow solid. MS ESI calculated for C₂₇H₃₄F₃N₅O₃ [M+H]⁺, 534.26, found 534.25. ¹H NMR (300 MHz, DMSO-d₆) δ 8.10 (s, 1H), 7.44-7.40 (m, 1H), 7.32-7.31 (m, 1H), 7.11-7.08 (m, 1H), 5.96 (s, 1H), 5.60 (s, 1H), 5.38-5.37 (m, 1H), 4.08-3.94 (m, 2H), 3.89-3.85 (m, 1H), 3.76-3.65 (m, 5H), 3.54-3.49 (m, 2H), 3.39-3.25 (m, 5H), 3.04-3.00 (m, 1H), 2.49-2.42 (m, 3H), 2.15-2.05 (m, 4H), 1.71-1.61 (m, 1H), 1.42 (d, J=6.3 Hz, 3H). ¹⁹F NMR (376 MHz, DMSO-d₆) δ-63.12 (3F).

### Example 74: (3S)-N-[4-methyl-3-[2-(morpholin-4-yl)-6-[[(2S)-1,1,1-trifluoro-3-hydroxypropan-2-yl]amino]pyridin-4-yl]phenyl]-3-(2,2,2-trifluoroethyl)pyrrolidine-1-carboxamide

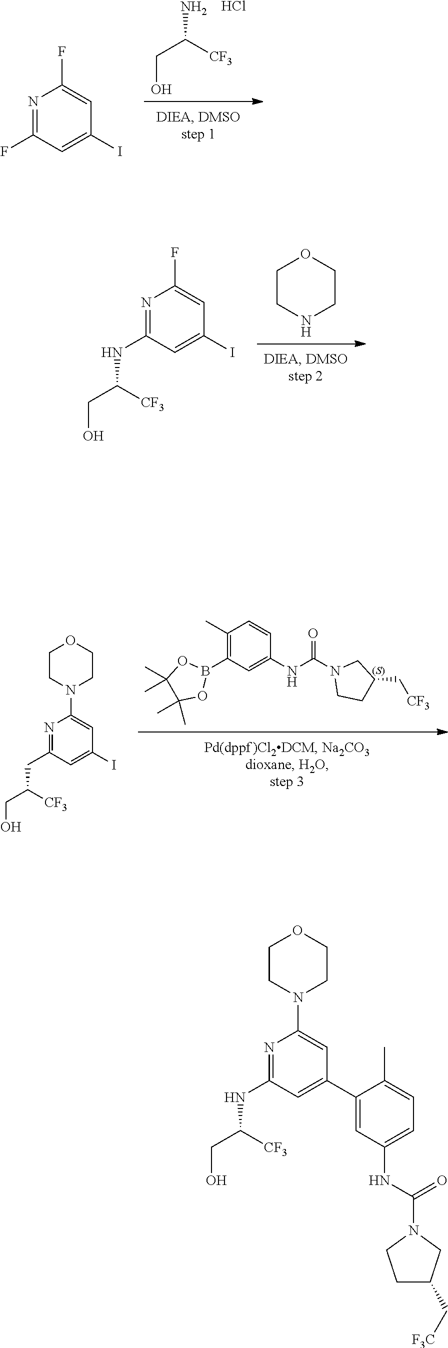

Preparation 74A: (2S)-3,3,3-trifluoro-2-[(6-fluoro-4-iodopyridin-2-yl)amino]propan-1-ol

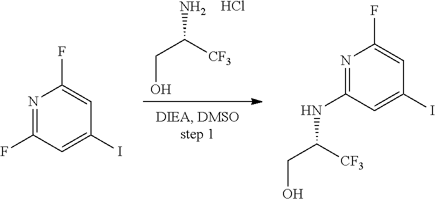 [SMILES: Cl ; Fc1cc(I)cc(F)n1 ; N[C@@H](CO)C(F)(F)F ; OC[C@H](Nc1cc(I)cc(F)n1)C(F)(F)F]

To a stirred mixture of 2,6-difluoro-4-iodopyridine (300 mg, 1.245 mmol) and (2S)-2-amino-3,3,3-trifluoropropan-1-ol (209 mg, 1.618 mmol) in DMSO (4 mL) was added K₂CO₃ (516 mg, 3.735 mmol) at room temperature. The resulting mixture was stirred for 2 h at 70° C. The resulting mixture was extracted with EtOAc (3×100 mL). The combined organic layers were washed with brine (3×100 mL), dried over anhydrous Na₂SO₄. After filtration, the filtrate was concentrated under reduced pressure. The residue was purified by silica gel column chromatography, eluted with EtOAc in PE (0% to 30%) to afford (2S)-3,3,3-trifluoro-2-[(6-fluoro-4-iodopyridin-2-yl)amino]propan-1-ol (20 mg, 5%) as a white solid. MS ESI calculated for C₈H₇F₄IN₂O [M+H]⁺, 350.95, found 350.95. ¹H NMR (300 MHz, chloroform-d) δ 6.80 (s, 1H), 6.68 (d, J=2.7 Hz, 1H), 5.15 (d, J=9.5 Hz, 1H), 4.90-4.74 (m, 1H), 4.07 (dd, J=12.0, 3.7 Hz, 1H).

Preparation 74B: (2S)-3,3,3-trifluoro-2-[[4-iodo-6-(morpholin-4-yl)pyridin-2-yl]amino]propan-1-ol

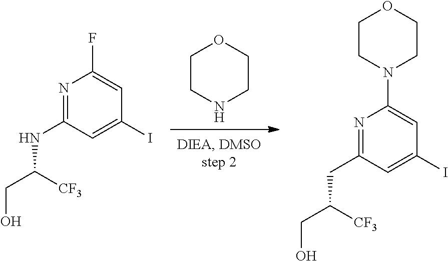 [SMILES: OC[C@H](Nc1cc(I)cc(F)n1)C(F)(F)F ; C1COCCN1 ; OC[C@H](Cc1cc(I)cc(N2CCOCC2)n1)C(F)(F)F]

To a stirred solution of (2S)-3,3,3-trifluoro-2-[(6-fluoro-4-iodopyridin-2-yl)amino]propan-1-ol (20 mg, 0.057 mmol) and morpholine (5 mg, 0.063 mmol) in DMSO (2 mL) was added K₂CO₃(24 mg, 0.171 mmol) at room temperature. The resulting mixture was stirred for 16 h at 70° C. The mixture was allowed to cool down to room temperature. The resulting mixture was diluted with water (20 mL) and extracted with EtOAc (3×30 mL). The combined organic layers were washed with brine (3×30 mL), dried over anhydrous Na₂SO₄. After filtration, the filtrate was concentrated under reduced pressure. The residue was purified by Prep-TLC (PE/EtOAc=2/1) to afford (2S)-3,3,3-trifluoro-2-[[4-iodo-6-(morpholin-4-yl)pyridin-2-yl]amino]propan-1-ol (10 mg, 17%) as a white solid. MS ESI calculated for C₁₂H₁₅F₃IN₃O₂[M+H]⁺, 418.02, found 417.85. ¹H NMR (300 MHz, chloroform-d) δ 6.55 (d, J=8.9 Hz, 2H), 4.54 (dd, J=11.2, 3.9 Hz, 1H), 4.36-4.25 (m, 1H), 3.88-3.74 (m, 4H), 3.66 (d, J=3.9 Hz, 1H), 3.54-3.38 (m, 4H).

### Example 74: (3S)-N-[4-methyl-3-[2-(morpholin-4-yl)-6-[[(2S)-1,1,1-trifluoro-3-hydroxypropan-2-yl]amino]pyridin-4-yl]phenyl]-3-(2,2,2-trifluoroethyl)pyrrolidine-1-carboxamide

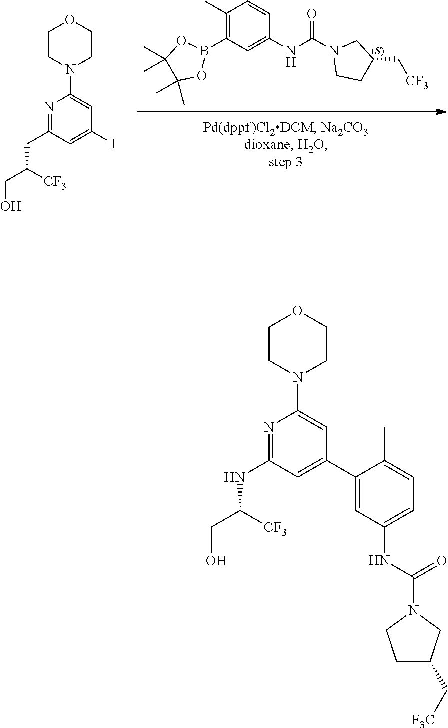

To a stirred mixture of (2S)-3,3,3-trifluoro-2-[[4-iodo-6-(morpholin-4-yl)pyridin-2-yl]amino]propan-1-ol (20 mg, 0.048 mmol) and (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (21 mg, 0.053 mmol), dioxane (1 mL) and H₂O (0.25 mL) were added Pd(dppf)Cl₂·CH₂Cl₂ (4 mg, 0.005 mmol) and Na₂CO₃ (15 mg, 0.144 mmol) at room temperature. The resulting mixture was stirred for 16 h at 60° C. under nitrogen atmosphere. The resulting mixture was concentrated under vacuum. The residue was purified by silica gel column chromatography, eluted with PE/EtOAc (1/1). The residue was purified by Prep-TLC (PE/EA/EtOH=8/3/1) to afford (3S)-N-[4-methyl-3-[2-(morpholin-4-yl)-6-[[(2S)-1,1,1-trifluoro-3-hydroxypropan-2-yl]amino]pyridin-4-yl]phenyl]-3-(2,2,2-trifluoroethyl)pyrrolidine-1-carboxamide (7 mg, 15%) as a white solid. MS ESI calculated for C₂₆H₃₁F₆N₅O₃[M+H]⁺, 575.23, found 575.56. ¹H NMR (400 MHz, DMSO-d₆) δ 8.12 (s, 1H), 7.42 (dd, J=8.2, 2.4 Hz, 1H), 7.36 (d, J=2.3 Hz, 1H), 7.10 (d, J=8.4 Hz, 1H), 6.70 (d, J=9.0 Hz, 1H), 5.94 (d, J=1.0 Hz, 1H), 5.88 (d, J=1.0 Hz, 1H), 5.12 (t, J=5.7 Hz, 1H), 4.99-4.88 (m, 1H), 3.77-3.62 (m, J=6.2 Hz, 7H), 3.52 (t, J=2.1 Hz, 1H), 3.47-3.34 (m, 5H), 3.02 (t, J=9.4 Hz, 1H), 2.49-2.34 (m, 3H), 2.16 (s, 3H), 2.12-2.05 (m, 1H), 1.73-1.59 (m, 1H). ¹⁹F NMR (376 MHz, DMSO-d₆) δ-64.96 (3F), −73.37 (3F).

### Example 75: (3S)-N-[3-[2-(2-hydroxy-2-methylpropanamido)-6-(morpholin-4-yl)pyridin-4-yl]-4-methylphenyl]-3-(2,2,2-trifluoroethyl)pyrrolidine-1-carboxamide

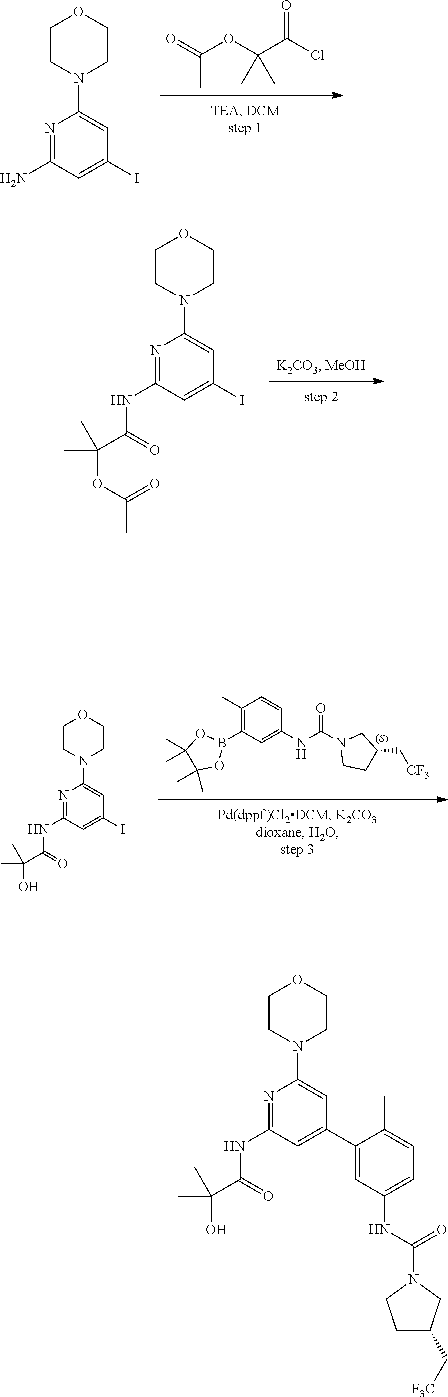

Preparation 75A: 1-[[4-iodo-6-(morpholin-4-yl)pyridin-2-yl]carbamoyl]-1-methylethyl acetate

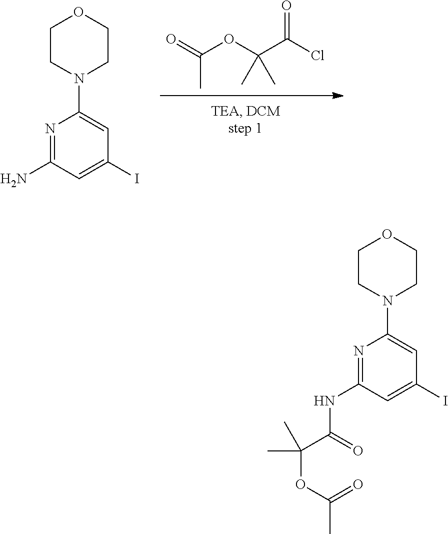

To a stirred mixture of 4-iodo-6-(morpholin-4-yl)pyridin-2-amine (250 mg, 0.819 mmol) and 1-chloro-2-methyl-1-oxopropan-2-yl acetate (202 mg, 1.229 mmol) in DCM (3 mL) was added TEA (248 mg, 2.458 mmol) at room temperature. The reaction mixture was stirred for 6 h at room temperature. The resulting mixture was diluted with water (20 mL). The resulting mixture was extracted with EtOAc (3×20 mL). The combined organic layers were washed with brine (2×30 mL), dried over anhydrous Na₂SO₄. After filtration, the filtrate was concentrated under reduced pressure to afford 1-[[4-iodo-6-(morpholin-4-yl)pyridin-2-yl]carbamoyl]-1-methylethyl acetate (400 mg, crude) as a yellow solid. MS ESI calculated for C₁₅H₂₀lN₃O₄[M+H]⁺, 434.05, found 434.00.

Preparation 75B: 2-hydroxy-N-[4-iodo-6-(morpholin-4-yl)pyridin-2-yl]-2-methylpropanamide

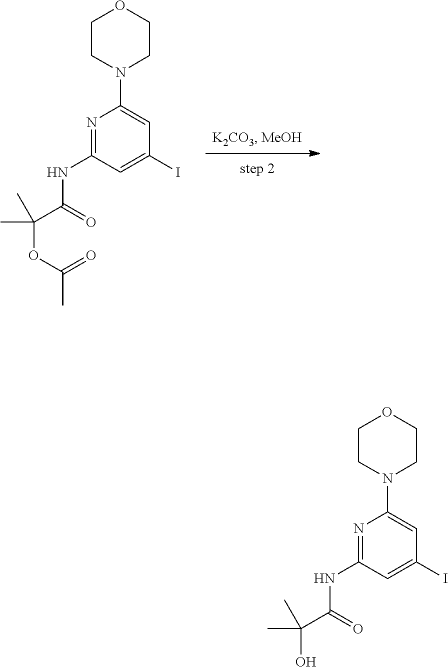 [SMILES: CC(=O)OC(C)(C)C(=O)Nc1cc(I)cc(N2CCOCC2)n1 ; CC(C)(O)C(=O)Nc1cc(I)cc(N2CCOCC2)n1]

To a stirred mixture of 1-[[4-iodo-6-(morpholin-4-yl)pyridin-2-yl]carbamoyl]-1-methylethyl acetate (370 mg, 0.854 mmol) in MeOH (4 mL) was added K₂CO₃ (354 mg, 2.562 mmol) at room temperature. The reaction mixture was stirred for 16 h at room temperature. The resulting mixture was diluted with water (30 mL). The resulting mixture was extracted with EtOAc (3×40 mL). The combined organic layers were washed with brine (3×40 mL), dried over anhydrous Na₂SO₄. After filtration, the filtrate was concentrated under reduced pressure. The residue was purified by reverse phase chromatography, eluted with 65% to 80% CH₃CN in water (with 0.5% NH₄HCO₃). The fractions containing the desired product were collected and concentrated under reduced pressure to afford 2-hydroxy-N-[4-iodo-6-(morpholin-4-yl)pyridin-2-yl]-2-methylpropanamide (180 mg, 53%) as an off white solid. MS ESI calculated for C₁₃H₁₈IN₃O₃[M+H]⁺, 392.04, found 391.95; ¹H NMR (400 MHz, DMSO-d₆) δ 9.09 (s, 1H), 7.81 (d, J=1.0 Hz, 1H), 6.96 (d, J=1.1 Hz, 1H), 6.03 (s, 1H), 3.66 (dd, J=5.8, 3.9 Hz, 4H), 3.43 (dd, J=5.7, 3.9 Hz, 4H), 1.34 (s, 6H).

### Example 75: (3S)-N-[3-[2-(2-hydroxy-2-methylpropanamido)-6-(morpholin-4-yl)pyridin-4-yl]-4-methylphenyl]-3-(2,2,2-trifluoroethyl)pyrrolidine-1-carboxamide

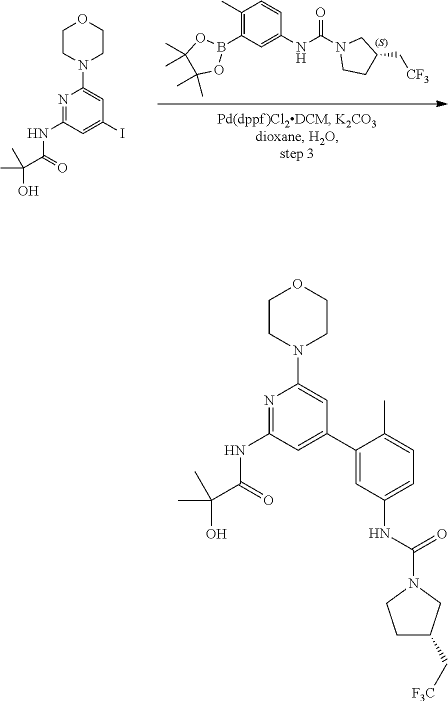

To a stirred mixture of 2-hydroxy-N-[4-iodo-6-(morpholin-4-yl)pyridin-2-yl]-2-methylpropanamide (130 mg, 0.332 mmol), (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (164 mg, 0.399 mmol) and K₂CO₃ (137 mg, 0.997 mmol) in dioxane (2 mL) and H₂O (0.5 mL) was added Pd(dppf)Cl₂·CH₂Cl₂ (27 mg, 0.033 mmol). The reaction mixture was stirred for 2 h at 80° C. under N₂ atmosphere. The resulting mixture was diluted with water (20 mL). The resulting mixture was extracted with EtOAc (3×30 mL). The combined organic layers were washed with brine (2×30 mL), dried over anhydrous Na₂SO₄. After filtration, the filtrate was concentrated under reduced pressure. The residue was purified by silica gel column chromatography, eluted with 45% PE in EA. The fractions containing the desired product were collected and concentrated under reduced pressure. The crude was purified by reverse phase chromatography, eluted with CH₃CN in water (with 10 mmol/mL NH₄HCO₃). The fractions containing the desired product were collected and concentrated under reduced pressure to afford (3S)-N-[3-[2-(2-hydroxy-2-methylpropanamido)-6-(morpholin-4-yl)pyridin-4-yl]-4-methylphenyl]-3-(2,2,2-trifluoroethyl)pyrrolidine-1-carboxamide (138 mg, 73%) as a white solid. MS ESI calculated for C₂₇H₃₄F₃N₅O₄[M+H]⁺, 550.26, found 550.25; ¹H NMR (400 MHz, DMSO-d₆) δ 9.16 (s, 1H), 8.18 (s, 1H), 7.49 (dd, J=8.3, 2.3 Hz, 1H), 7.40 (dd, J=6.3, 1.6 Hz, 2H), 7.15 (d, J=8.4 Hz, 1H), 6.49 (d, J=1.1 Hz, 1H), 6.03 (s, 1H), 3.73-3.63 (m, 5H), 3.58-3.43 (m, 5H), 3.33-3.26 (m, 1H), 3.03 (t, J=9.4 Hz, 1H), 2.49-2.33 (m, 3H), 2.18 (s, 3H), 2.08 (q, J=4.5, 3.8 Hz, 1H), 1.66 (dq, J=11.9, 9.7 Hz, 1H), 1.35 (s, 6H).

### Example 76: (3S)-N-[2-fluoro-5-(2-[[(2R)-1-hydroxypropan-2-yl]amino]-6-(morpholin-4-yl)pyridin-4-yl)-4-methylphenyl]-3-(2,2,2- trifluoroethyl)pyrrolidine-1-carboxamide

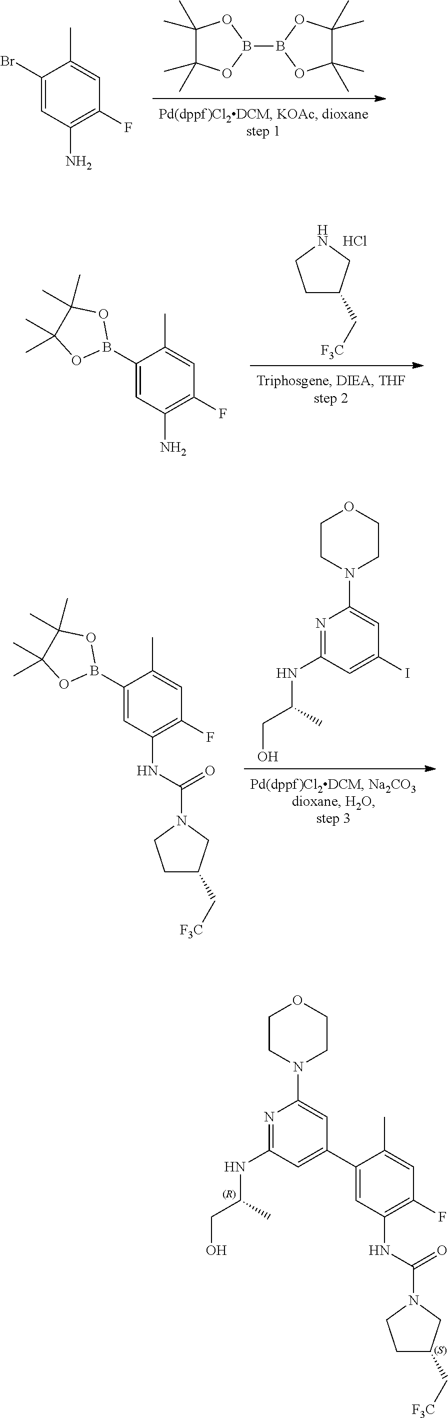

Preparation 76A: 2-fluoro-4-methyl-5-(4,4,5,5-tetramethyl-1,3,2-dioxaborolan-2-yl)aniline

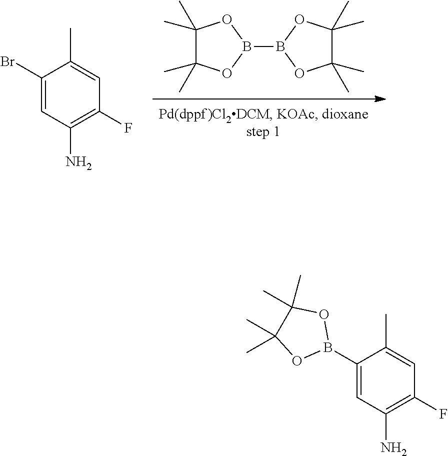

A mixture of 5-bromo-2-fluoro-4-methylaniline (50 g, 245.048 mmol), 4,4,5,5-tetramethyl-2-(tetramethyl-1,3,2-dioxaborolan-2-yl)-1,3,2-dioxaborolane (75 g, 294.057 mmol), KOAc (72 g, 735.143 mmol,) and Pd(dppf)Cl₂·CH₂Cl₂ (10 g, 12.252 mmol) in 1,4-dioxane (600 mL) was stirred for 16 h at 100° C. under argon atmosphere. The resulting mixture was concentrated under reduced pressure. The residue was purified by silica gel column chromatography, eluted with EtOAc in PE (0-30%) to afford 2-fluoro-4-methyl-5-(4,4,5,5-tetramethyl-1,3,2-dioxaborolan-2-yl)aniline (57 g, 93%) as an off-white solid. MS ESI calculated for C₁₃H₁₉BFNO₂ [M+H]⁺, 252.15, found 252.10.

Preparation 76B: (3S)-N-[2-fluoro-4-methyl-5-(4,4,5,5-tetramethyl-1,3,2-dioxaborolan-2-yl)phenyl]-3-(2,2,2-trifluoroethyl)pyrrolidine-1-carboxamide

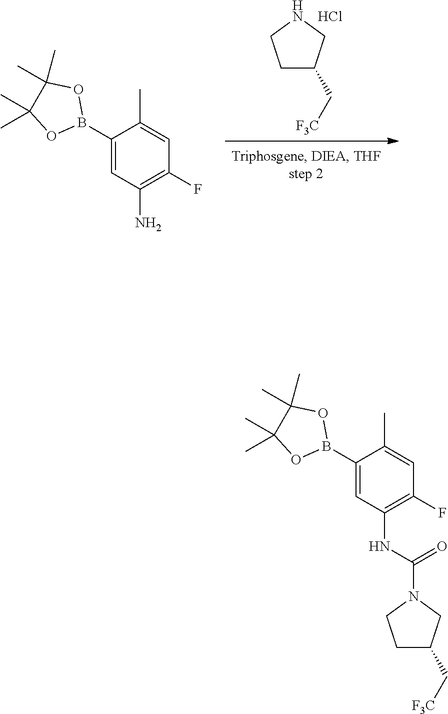 [SMILES: Cl ; Cc1cc(F)c(N)cc1B1OC(C)(C)C(C)(C)O1 ; FC(F)(F)C[C@@H]1CCNC1 ; Cc1cc(F)c(NC(=O)N2CC[C@@H](CC(F)(F)F)C2)cc1B1OC(C)(C)C(C)(C)O1]

A mixture of 2-fluoro-4-methyl-5-(4,4,5,5-tetramethyl-1,3,2-dioxaborolan-2-yl)aniline (2.00 g, 7.965 mmol, 1.00 equiv), DIEA (5.15 g, 0.040 mmol, 5 equiv) and Triphosgene (0.95 g, 0.003 mmol, 0.4 equiv) in THF (19 mL) was stirred for 30 min at room temperature under nitrogen atmosphere. To the above mixture was added (3S)-3-(2,2,2-trifluoroethyl)pyrrolidine hydrochloride (1.51 g, 0.008 mmol, 1.00 equiv). The resulting mixture was stirred for additional 3 h at room temperature. The resulting mixture was concentrated under vacuum. The residue was purified by silica gel column chromatography, eluted with PE/EtOAc/EtOH (4/3/1) to afford (3S)-N-[2-fluoro-4-methyl-5-(4,4,5,5-tetramethyl-1,3,2-dioxaborolan-2-yl)phenyl]-3-(2,2,2-trifluoroethyl)pyrrolidine-1-carboxamide (3.2 g, 75%) as an off-white solid. MS ESI calculated for C₂₀H₂₇BF₄N₂O₃ [M+H]⁺, 431.21, found 431.25. ¹H NMR (400 MHz, Chloroform-d) δ 8.38 (d, J=9.3 Hz, 1H), 6.88 (dd, J=12.3, 0.8 Hz, 1H), 6.20 (d, J=3.2 Hz, 1H), 3.83 (dd, J=9.9, 7.5 Hz, 1H), 3.67-3.63 (m, 1H), 3.46 (td, J=9.7, 6.8 Hz, 1H), 3.13 (t, J=9.5 Hz, 1H), 2.66-2.51 (m, 1H), 2.46 (s, 3H), 2.35-2.17 (m, 3H), 1.78-1.74 (m, 1H), 1.33 (s, 12H).

### Example 76: (3S)-N-[2-fluoro-5-(2-[[(2R)-1-hydroxypropan-2-yl]amino]-6-(morpholin-4-yl)pyridin-4-yl)-4-methylphenyl]-3-(2,2,2-trifluoroethyl)pyrrolidine-1-carboxamide

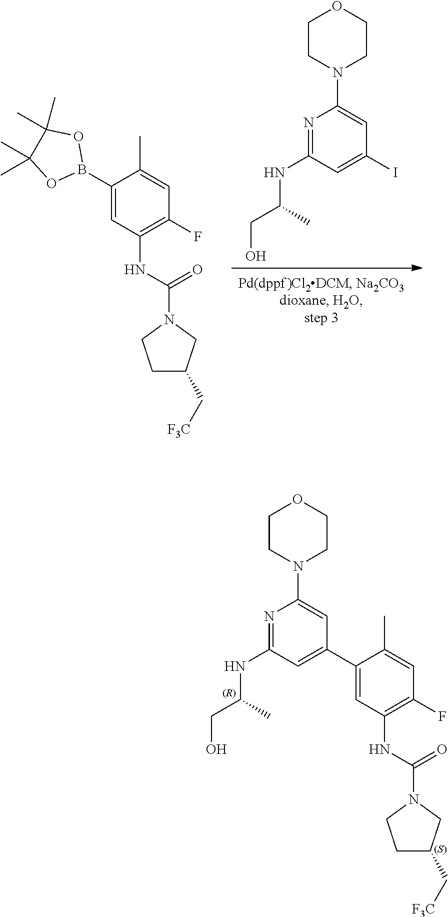

To a stirred solution of (2R)-2-[[4-iodo-6-(morpholin-4-yl)pyridin-2-yl]amino]propan-1-ol (130 mg, 0.358 mmol) and (3S)-N-[2-fluoro-4-methyl-5-(4,4,5,5-tetramethyl-1,3,2-dioxaborolan-2-yl)phenyl]-3-(2,2,2-trifluoroethyl)pyrrolidine-1-carboxamide (154 mg, 0.358 mmol) in 1,4-dioxane (2 mL) and H₂O (0.4 mL) were added Na₂CO₃ (113 mg, 1.074 mmol) and Pd(dppf)Cl₂·CH₂Cl₂ (29 mg, 0.036 mmol) at room temperature under nitrogen atmosphere. The resulting mixture was stirred for 16 h at 60° C. under nitrogen atmosphere. The mixture was allowed to cool down to room temperature. The reaction was quenched by the addition of water (20 mL) at room temperature. The resulting mixture was extracted with EtOAc (3×20 mL). The combined organic layers were washed with brine (3×20 mL), dried over anhydrous Na₂SO₄. After filtration, the filtrate was concentrated under reduced pressure. The residue was purified by silica gel column chromatography, eluted with PE/EtOAc (1/1) to afford (3S)-N-[2-fluoro-5-(2-[[(2R)-1-hydroxypropan-2-yl]amino]-6-(morpholin-4-yl)pyridin-4-yl)-4-methylphenyl]-3-(2,2,2-trifluoroethyl)pyrrolidine-1-carboxamide (105 mg, 54%) as a light yellow solid. MS ESI calculated for C₂₆H₃₃F₄N₅O₃[M+H]⁺, 540.25, found 540.15. ¹H NMR (400 MHz, DMSO-d₆) δ 7.86 (s, 1H), 7.29 (d, J=8.3 Hz, 1H), 7.10 (d, J=11.7 Hz, 1H), 5.99 (d, J=7.5 Hz, 1H), 5.77-5.71 (m, 2H), 4.64 (t, J=5.6 Hz, 1H), 3.92-3.84 (m, 1H), 3.67 (dd, J=5.7, 3.9 Hz, 5H), 3.57-3.44 (m, 2H), 3.41-3.32 (m, 5H), 3.03 (t, J=9.4 Hz, 1H), 2.51-2.35 (m, 3H), 2.20 (s, 3H), 2.13-2.05 (m, 1H), 1.74-1.62 (m, 1H), 1.24 (s, 1H), 1.12 (d, J=6.6 Hz, 3H). ¹⁹F NMR (376 MHz, DMSO-d₆) δ-63.39 (3F), −125.64 (1F).

### Example 77: N-[2-fluoro-5-(2-[[(2R)-1-hydroxypropan-2-yl]amino]-6-(morpholin-4-yl)pyridin-4-yl)-4-methylphenyl]-3-(2,2,2-trifluoroethyl)-2,5-dihydropyrrole-1-carboxamide

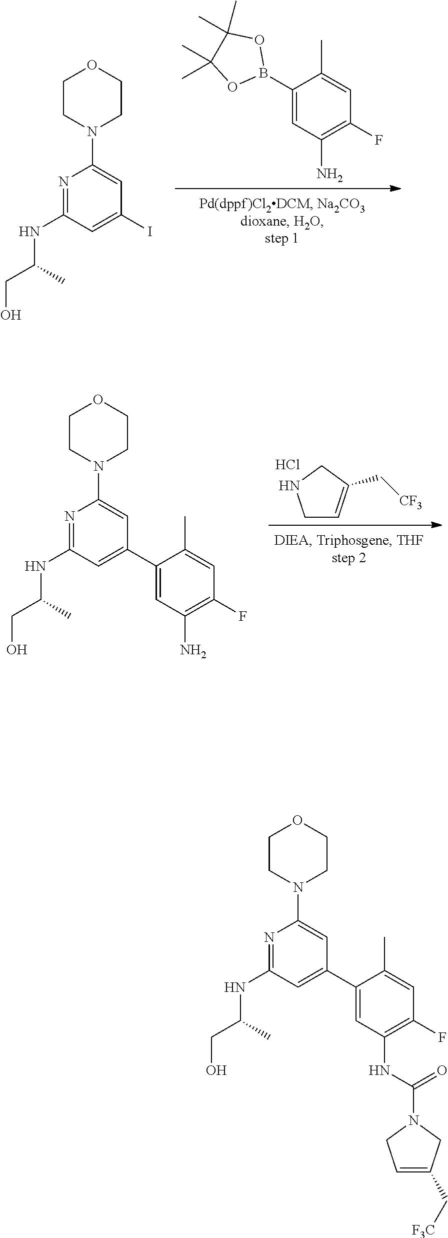

Preparation 77A: (2R)-2-[[4-(5-amino-4-fluoro-2-methylphenyl)-6-(morpholin-4-yl)pyridin-2-yl]amino]propan-1-ol

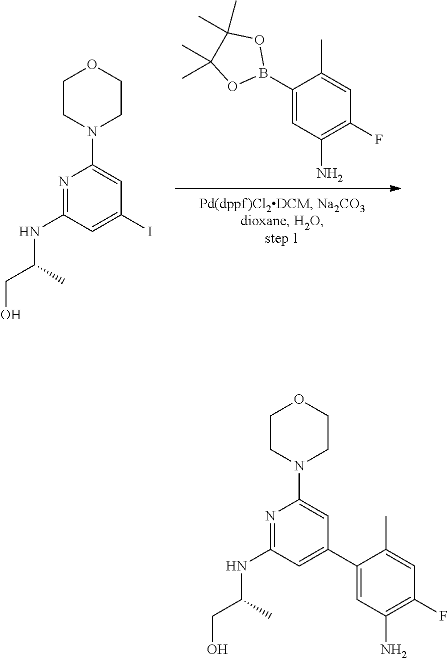 [SMILES: C[C@H](CO)Nc1cc(I)cc(N2CCOCC2)n1 ; Cc1cc(F)c(N)cc1B1OC(C)(C)C(C)(C)O1 ; Cc1cc(F)c(N)cc1-c1cc(N[C@H](C)CO)nc(N2CCOCC2)c1]

To a stirred solution of (2R)-2-[[4-iodo-6-(morpholin-4-yl)pyridin-2-yl]amino]propan-1-ol (200 mg, 0.551 mmol) and 2-fluoro-4-methyl-5-(4,4,5,5-tetramethyl-1,3,2-dioxaborolan-2-yl)aniline (138 mg, 0.551 mmol) in 1,4-dioxane (2 mL) and H₂O (0.4 mL) were added Na₂CO₃ (175 mg, 1.652 mmol) and Pd(dppf)Cl₂ CH₂Cl₂(45 mg, 0.055 mmol) at room temperature under nitrogen atmosphere. The resulting mixture was stirred for 1 h at 80° C. under nitrogen atmosphere. The mixture was allowed to cool down to room temperature. The reaction was quenched by the addition of water (20 mL) at room temperature. The resulting mixture was extracted with EtOAc (3×50 mL). The combined organic layers were washed with brine (3×50 mL), dried over anhydrous Na₂SO₄. After filtration, the filtrate was concentrated under reduced pressure. The residue was purified by silica gel column chromatography, eluted with PE/EtOAc/EtOH(6/3/1) to afford (2R)-2-[[4-(5-amino-4-fluoro-2-methylphenyl)-6-(morpholin-4-yl)pyridin-2-yl]amino]propan-1-ol (140 mg, 38%) as an off-white solid. MS ESI calculated for C₁₉H₂₅FN₄O₂ [M+H]⁺, 361.20, found 361.15. ¹H NMR (300 MHz, chloroform-d) δ 6.88 (d, J=11.9 Hz, 1H), 6.64 (d, J=9.2 Hz, 1H), 5.81 (d, J=14.5 Hz, 2H), 4.08 (s, 1H), 3.88-3.73 (m, 5H), 3.68-3.53 (m, 3H), 3.49 (s, 4H), 2.17 (s, 3H), 1.25 (d, J=6.7 Hz, 3H). ¹⁹F NMR (282 MHz, chloroform-d) δ-136.65.

### Example 77: N-[2-fluoro-5-(2-[[(2R)-1-hydroxypropan-2-yl]amino]-6-(morpholin-4-yl)pyridin-4-yl)-4-methylphenyl]-3-(2,2,2-trifluoroethyl)-2,5-dihydropyrrole-1-carboxamide

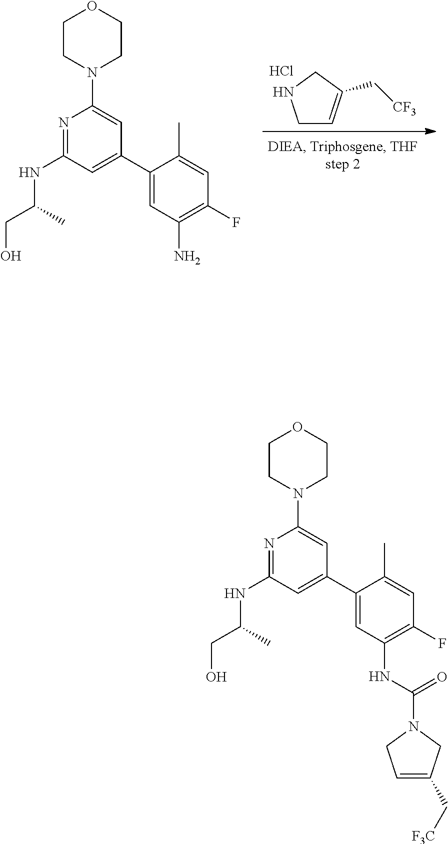

A mixture of (2R)-2-[[4-(5-amino-4-fluoro-2-methylphenyl)-6-(morpholin-4-yl)pyridin-2-yl]amino]propan-1-ol (60 mg, 0.166 mmol), DIEA (0.14 mL, 1.084 mmol) and triphosgene (20 mg, 0.067 mmol) in THF (6 mL) was stirred for 30 min at room temperature. To the above mixture was added 3-(2,2,2-trifluoroethyl)-2,5-dihydro-1H-pyrrole hydrochloride (31 mg, 0.166 mmol) at room temperature. The resulting mixture was stirred for additional 3 h at room temperature. The resulting mixture was concentrated under vacuum. The residue was purified by silica gel column chromatography, eluted with (PE/EtOAc/EtOH=8/3/1) to afford mixture. The residue was purified by reverse flash chromatography with the following conditions: column, C18 silica gel; mobile phase, CH₃CN in water (0.1% NH₄HCO₃), 10% to 50% gradient in 20 min; detector, UV 254 nm to afford N-[2-fluoro-5-(2-[[(2R)-1-hydroxypropan-2-yl]amino]-6-(morpholin-4-yl)pyridin-4-yl)-4-methylphenyl]-3-(2,2,2-trifluoroethyl)-2,5-dihydropyrrole-1-carboxamide (5 mg, 6%) as a white solid. MS ESI calculated for C₂₆H₃₁F₄N₅O₃[M+H]⁺, 538.24, found 538.05. ¹H NMR (400 MHz, DMSO-d₆) δ 7.97 (s, 1H), 7.31 (d, J=8.3 Hz, 1H), 7.11 (d, J=11.7 Hz, 1H), 5.99 (d, J=7.5 Hz, 1H), 5.93 (s, 1H), 5.77-5.72 (m, 2H), 4.64 (t, J=5.5 Hz, 1H), 4.20 (s, 4H), 3.94-3.85 (m, 1H), 3.67 (t, J=4.7 Hz, 4H), 3.53-3.45 (m, 1H), 3.37 (t, J=4.9 Hz, 4H), 3.28 (d, J=13.8 Hz, 3H), 2.21 (s, 3H), 1.12 (d, J=6.6 Hz, 3H). ¹⁹F NMR (376 MHz, DMSO-d₆) δ-66.30 (3F), −125.50 (1F).

### Example 78: (3S)-N-[3-[2-(2,5-dihydrofuran-3-yl)-6-[[(2R)-1-hydroxypropan-2-yl]amino]pyridin-4-yl]-4-methylphenyl]-3-(2,2,2-trifluoroethyl)pyrrolidine-1-carboxamide

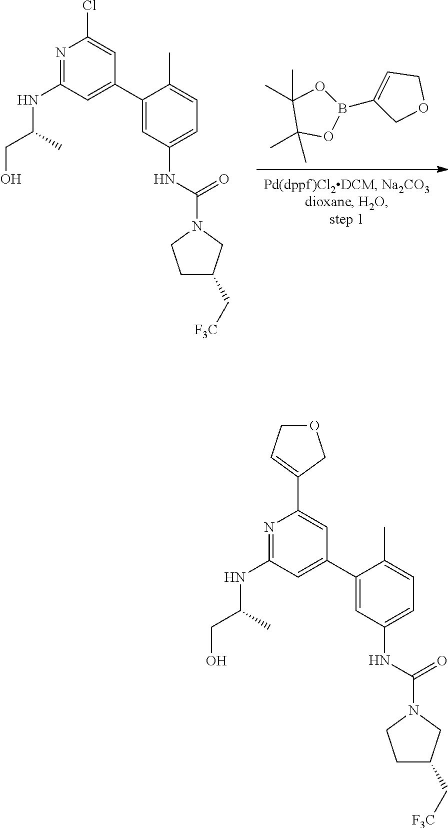 [SMILES: Cc1ccc(NC(=O)N2CC[C@@H](CC(F)(F)F)C2)cc1-c1cc(Cl)nc(N[C@H](C)CO)c1 ; CC1(C)OB(C2=CCOC2)OC1(C)C ; Cc1ccc(NC(=O)N2CC[C@@H](CC(F)(F)F)C2)cc1-c1cc(N[C@H](C)CO)nc(C2=CCOC2)c1]

To a stirred mixture of (3S)-N-[3-(2-chloro-6-[[(2R)-1-hydroxypropan-2-yl]amino]pyridin-4-yl)-4-methylphenyl]-3-(2,2,2-trifluoroethyl)pyrrolidine-1-carboxamide (500 mg, 1.062 mmol) and 2-(2,5-dihydrofuran-3-yl)-4,4,5,5-tetramethyl-1,3,2-dioxaborolane (416 mg, 2.124 mmol) in 1,4-dioxane (6 mL) and H₂O (0.6 mL) were added Pd(dppf)Cl₂·CH₂Cl₂ (87 mg, 0.106 mmol) and Na₂CO₃ (338 mg, 3.185 mmol). The resulting mixture was stirred for 2 h at 80° C. under nitrogen atmosphere. The resulting mixture was concentrated under reduced pressure. The residue was purified by silica gel column chromatography, eluted with (PE/EtOAc/EtOH=12/3/1). The residue was purified by reverse phase flash chromatography with the following conditions: Column: Spherical C18, 20-40 um, 120 g; Mobile Phase A: water (5 mM NH₄HCO₃); Mobile Phase B: CH₃CN; Flow rate: 50 mL/min; Gradient: 35%-55% B gradient in 30 min; Detector: 220 nm. The fractions containing the desired product were collected at 46% B and concentrated under reduced pressure to afford (3S)-N-[3-[2-(2,5-dihydrofuran-3-yl)-6-[[(2R)-1-hydroxypropan-2-yl]amino]pyridin-4-yl]-4-methylphenyl]-3-(2,2,2-trifluoroethyl)pyrrolidine-1-carboxamide (320 mg, 60%) as a white solid. MS ESI calculated for C₂₆H₃₁F₃N₄O₃[M+H]⁺, 505.23 found 505.25. ¹H NMR (300 MHz, DMSO-d₆) δ 8.16 (s, 1H), 7.54-7.33 (m, 2H), 7.14 (d, J=8.4 Hz, 1H), 6.67-6.56 (m, 2H), 6.32 (t, J=2.0 Hz, 2H), 4.91 (td, J=5.0, 2.1 Hz, 2H), 4.75 (td, J=5.1, 2.1 Hz, 3H), 4.05-3.86 (m, 1H), 3.75-3.61 (m, 1H), 3.52 (dt, J=8.8, 2.5 Hz, 2H), 3.36 (s, 2H), 3.03 (t, J=9.3 Hz, 1H), 2.49-2.31 (m, 3H), 2.17 (s, 3H), 2.14-2.03 (m, 1H), 1.68 (d, J=10.4 Hz, 1H), 1.15 (d, J=6.6 Hz, 3H). ¹⁹F NMR (376 MHz, DMSO-d₆) δ-63.363 (3F).

### Example 79: (3S)-N-[3-(2-[[(2R)-1-hydroxypropan-2-yl]amino]-6-(oxolan-3-yl)pyridin-4-yl)-4-methylphenyl]-3-(2,2,2-trifluoroethyl)pyrrolidine-1-carboxamide

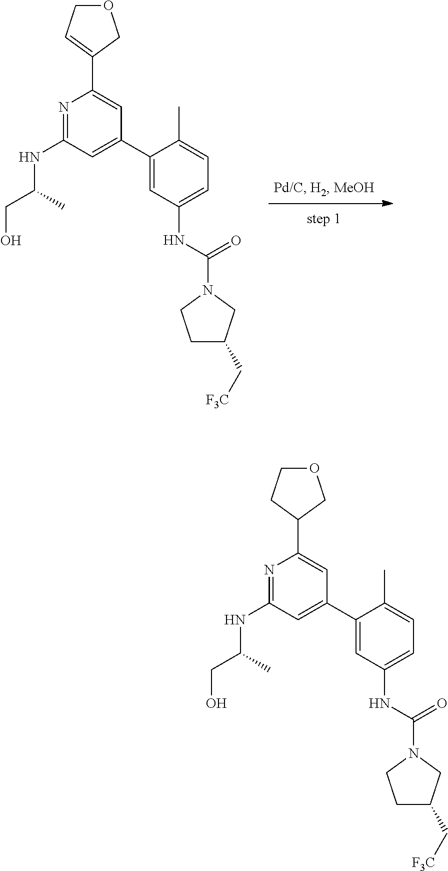 [SMILES: Cc1ccc(NC(=O)N2CC[C@@H](CC(F)(F)F)C2)cc1-c1cc(N[C@H](C)CO)nc(C2=CCOC2)c1 ; Cc1ccc(NC(=O)N2CC[C@@H](CC(F)(F)F)C2)cc1-c1cc(N[C@H](C)CO)nc(C2CCOC2)c1]

To a stirred mixture of (3S)-N-[3-[2-(2,5-dihydrofuran-3-yl)-6-[[(2R)-1-hydroxypropan-2-yl]amino]pyridin-4-yl]-4-methylphenyl]-3-(2,2,2-trifluoroethyl)pyrrolidine-1-carboxamide (100 mg, 0.198 mmol) in MeOH (5 mL) was added Pd/C (21 mg, 0.198 mmol). The resulting mixture was stirred for 16 h at room temperature under hydrogen atmosphere. The resulting mixture was filtered. The filter cake was washed with MeOH (3×20 mL). The filtrate was concentrated under reduced pressure. The residue was purified by reverse phase flash chromatography with the following conditions: Column: Spherical C18, 20-40 um, 120 g; Mobile Phase A: Water (plus 5 mmol/mL NH₄HCO₃); Mobile Phase B: CH₃CN; Flow rate: 60 mL/min; Gradient: 35%-55% B gradient in 25 min; Detector: 220 nm. The fractions containing the desired product were collected at 48% B and concentrated under reduced pressure to afford (3S)-N-[3-(2-[[(2R)-1-hydroxypropan-2-yl]amino]-6-(oxolan-3-yl)pyridin-4-yl)-4-methylphenyl]-3-(2,2,2-trifluoroethyl)pyrrolidine-1-carboxamide (79 mg, 79%) as a white solid. MS ESI calculated for C₂₆H₃₃F₃N₄O₃[M+H]⁺, 507.25 found 507.30. ¹H NMR (300 MHz, DMSO-d₆) δ 8.13 (s, 1H), 7.44 (dd, J=8.3, 2.3 Hz, 1H), 7.37 (d, J=2.3 Hz, 1H), 7.12 (d, J=8.3 Hz, 1H), 6.33 (d, J=1.3 Hz, 1H), 6.30-6.17 (m, 2H), 4.73 (t, J=5.5 Hz, 1H), 4.03 (t, J=7.8 Hz, 1H), 3.99-3.61 (m, 5H), 3.60-3.43 (m, 2H), 3.40-3.34 (m, 3H), 3.03 (t, J=9.3 Hz, 1H), 2.44 (t, J=10.1 Hz, 3H), 2.25-2.00 (m, 6H), 1.66 (p, J=9.8 Hz, 1H), 1.15 (d, J=6.5 Hz, 3H). ¹⁹F NMR (376 MHz, DMSO-d₆) δ-63.360 (3F).

### Example 80: (3S)-N-[3-(2-[[(2S)-1-hydroxypropan-2-yl]amino]-6-(1H-pyrazol-4-yl)pyridin-4-yl)-4-methylphenyl]-3-(2,2,2-trifluoroethyl)pyrrolidine-1-carboxamide

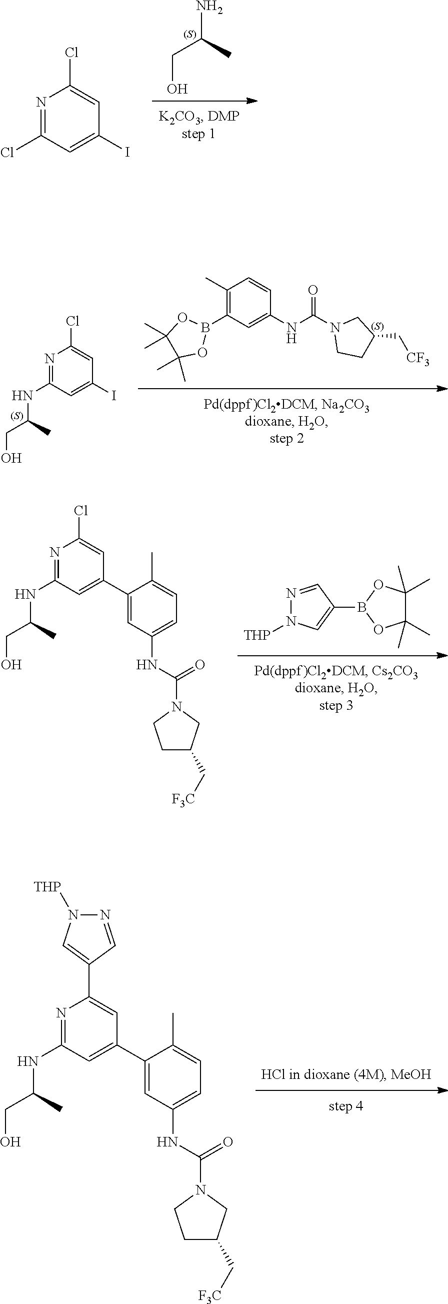

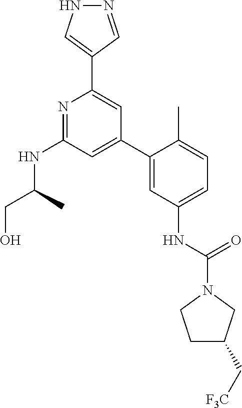 [SMILES: Cc1ccc(NC(=O)N2CC[C@@H](CC(F)(F)F)C2)cc1-c1cc(N[C@@H](C)CO)nc(-c2cn[nH]c2)c1]

Preparation 80A: (2S)-2-[(6-chloro-4-iodopyridin-2-yl)amino]propan-1-ol

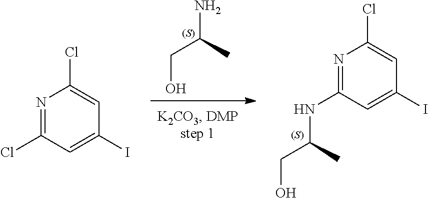

To a stirred solution of 2,6-dichloro-4-iodopyridine (5.00 g, 18.256 mmol) and K₂CO₃ (5.05 g, 36.540 mmol) in NMP (100 mL) was added (2S)-2-aminopropan-1-ol (1.37 g, 18.256 mmol) at rt ° C. The resulting mixture was stirred for 2 h at 150° C. The reaction was quenched by the addition of water (1200 mL). The resulting mixture was extracted with EtOAc (3×1000 mL). The combined organic layers were washed with saturated brine (2×500 mL), dried over anhydrous Na₂SO₄. After filtration, the filtrate was concentrated under reduced pressure. The residue was purified by silica gel column chromatography, eluted with PE/EtOAc (2/1) to afford (2S)-2-[(6-chloro-4-iodopyridin-2-yl)amino]propan-1-ol (1.3 g, 23%) as a yellow oil. MS ESI calculated for C8H10ClIN2O [M+H]⁺, 312.95, found 313.05. ¹H NMR (400 MHz, Chloroform-d) δ 7.05 (s, 1H), 6.97 (s, 1H), 4.02-3.94 (m, 1H), 3.78-3.74 (m, 1H), 3.63-3.58 (m, 1H), 1.26 (d, J=10.8 Hz, 3H).

Preparation 80B: (3S)-N-[3-(2-chloro-6-[[(2S)-1-hydroxypropan-2-yl]amino]pyridin-4-yl)-4-methylphenyl]-3-(2,2,2-trifluoroethyl)pyrrolidine-1-carboxamide

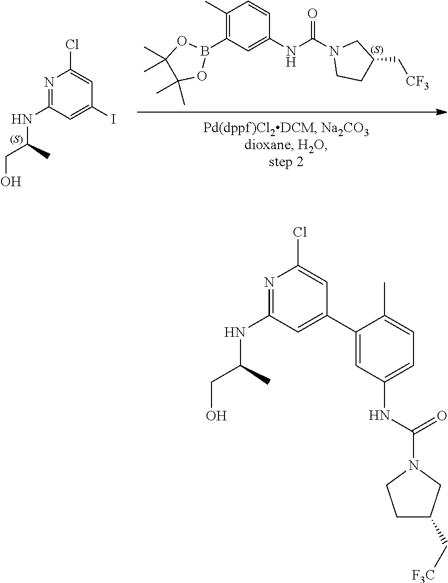

A mixture of (2S)-2-[(6-chloro-4-iodopyridin-2-yl)amino]propan-1-ol (300 mg, 0.960 mmol), (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (395 mg, 0.960 mmol), Pd(dppf)Cl₂·CH₂Cl₂ (78 mg, 0.096 mmol) and Na₂CO₃ (305 mg, 2.880 mmol) in dioxane (3 mL) and H₂O (0.75 mL) was stirred for 16 h at 60° C. under nitrogen atmosphere. The reaction was quenched by the addition of water (50 mL) at room temperature. The resulting mixture was extracted with EtOAc (2×100 mL). The combined organic layers were washed with brine (3×50 mL), dried over anhydrous Na₂SO₄. After filtration, the filtrate was concentrated under reduced pressure. The residue was purified by silica gel column chromatography, eluted with (PE/EtOAc/EtOH=8/3/1) to afford (3S)-N-[3-(2-chloro-6-[[(2S)-1-hydroxypropan-2-yl]amino]pyridin-4-yl)-4-methylphenyl]-3-(2,2,2-trifluoroethyl)pyrrolidine-1-carboxamide (410 mg, 85%) as a brown solid. MS ESI calculated for C₂₂H₂₆ClF₃N₄O₂ [M+H]⁺, 471.17, found 471.20. ¹H NMR (400 MHz, chloroform-d) δ 7.31-7.30 (m, 1H), 7.19-7.16 (m, 1H), 6.57 (s, 1H), 6.27-6.23 (m, 2H), 4.77 (s, 1H), 4.13-3.81 (m, 1H), 3.79-3.74 (m, 2H), 3.67-3.58 (m, 2H), 3.49-3.40 (m, 1H), 3.15-3.09 (m, 1H), 2.57-2.55 (m, 1H), 2.34-2.24 (m, 5H), 1.82-1.72 (m, 1H), 1.31-1.25 (m, 3H).

Preparation 80C: (3S)-N-[3-(2-chloro-6-[[(2S)-1-hydroxypropan-2-yl]amino]pyridin-4-yl)-4-methylphenyl]-3-(2,2,2-trifluoroethyl)pyrrolidine-1-carboxamide

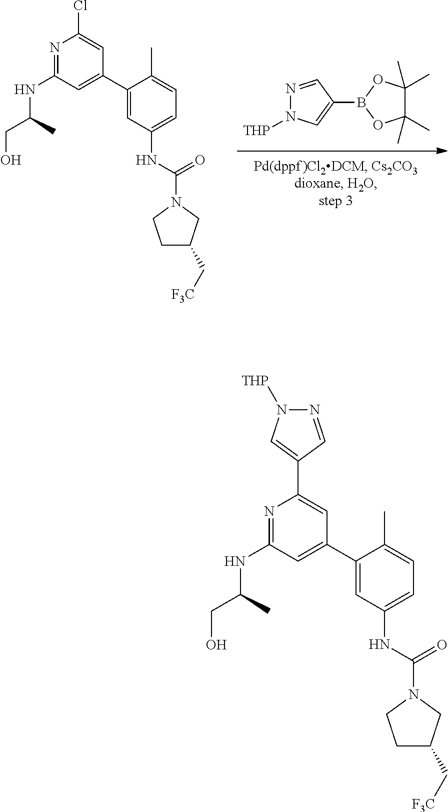

A mixture of (3S)-N-[3-(2-chloro-6-[[(2S)-1-hydroxypropan-2-yl]amino]pyridin-4-yl)-4-methylphenyl]-3-(2,2,2-trifluoroethyl)pyrrolidine-1-carboxamide (300 mg, 0.637 mmol), 1-(oxan-2-yl)-4-(4,4,5,5-tetramethyl-1,3,2-dioxaborolan-2-yl)pyrazole (354 mg, 1.274 mmol), Pd(dppf)Cl₂·CH₂Cl₂ (52 mg, 0.064 mmol) and Cs₂CO₃ (622 mg, 1.911 mmol) in dioxane (3 mL) and H₂O (0.3 mL) was stirred for 2 h at 80° C. under nitrogen atmosphere. The reaction was quenched by the addition of water (50 mL) at room temperature. The resulting mixture was extracted with EtOAc (3×100 mL). The combined organic layers were washed with brine (3×50 mL), dried over anhydrous Na₂SO₄. After filtration, the filtrate was concentrated under reduced pressure. The residue was purified by silica gel column chromatography, eluted with (PE/EtOAc/EtOH=8/3/1) to afford (3S)-N-[3-(2-[[(2S)-1-hydroxypropan-2-yl]amino]-6-[1-(oxan-2-yl)pyrazol-4-yl]pyridin-4-yl)-4-methylphenyl]-3-(2,2,2-trifluoroethyl)pyrrolidine-1-carboxamide (330 mg, 79%) as a light brown solid. MS ESI calculated for C₃₀H₃₇F₃N₆O₃ [M+H]⁺, 587.29, found 587.30. ¹H NMR (300 MHz, chloroform-d) δ 8.22 (s, 1H), 7.95 (s, 1H), 7.40-7.36 (m, 2H), 7.21-7.19 (m, 1H), 6.79 (s, 1H), 6.28-6.26 (m, 2H), 5.43-5.39 (m, 1H), 4.18-4.07 (m, 2H), 3.85-3.84 (m, 1H), 3.81-3.62 (m, 5H), 3.50-3.41 (m, 1H), 3.16-3.10 (m, 1H), 2.57 (m, 1H), 2.34-2.26 (m, 3H), 2.10-2.07 (m, 2H), 1.82-1.62 (m, 6H), 1.31-1.26 (m, 3H). ¹⁹F NMR (282 MHz, chloroform-d) δ-64.58-65.56 (3F).

### Example 80: (3S)-N-[3-(2-[[(2S)-1-hydroxypropan-2-yl]amino]-6-(1H-pyrazol-4-yl)pyridin-4-yl)-4-methylphenyl]-3-(2,2,2-trifluoroethyl)pyrrolidine-1-carboxamide

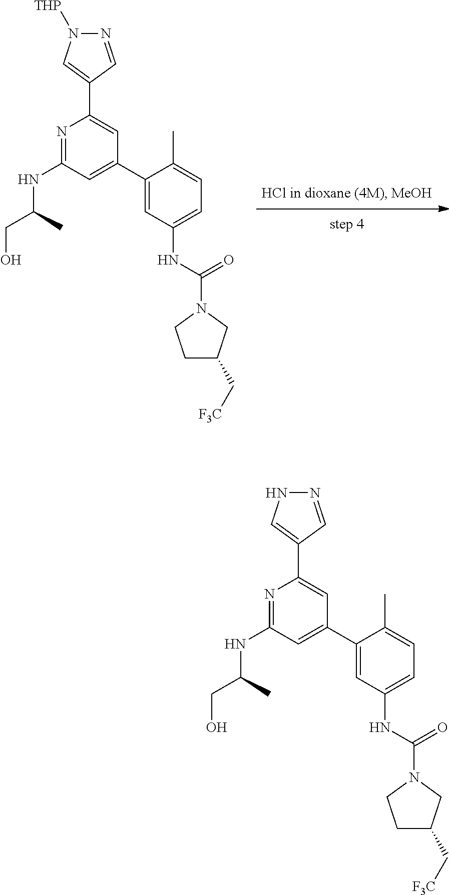

To a stirred mixture of (3S)-N-[3-(2-[[(2S)-1-hydroxypropan-2-yl]amino]-6-[1-(oxan-2-yl)pyrazol-4-yl]pyridin-4-yl)-4-methylphenyl]-3-(2,2,2-trifluoroethyl)pyrrolidine-1-carboxamide (330 mg, 0.563 mmol) in MeOH (3 mL) and THF (4 mL) was added HCl (gas) in 1,4-dioxane (1 mL) dropwise at 0° C. The resulting mixture was stirred for 30 min at room temperature. The reaction was quenched by the addition of sat. NaHCO₃ (aq.) (10 mL) at 0° C. The resulting mixture was extracted with EtOAc (2×20 mL). The combined organic layers were washed with brine (3×50 mL), dried over anhydrous Na₂SO₄. After filtration, the filtrate was concentrated under reduced pressure. The residue was purified by reverse flash chromatography with the following conditions: Mobile Phase A: water (10 mmol/L NH₄HCO₃), Mobile Phase B: CH₃CN; Flow rate:20 mL/min; Gradient:40 B to 65 B in 20 min. This resulted in (3S)-N-[3-(2-[[(2S)-1-hydroxypropan-2-yl]amino]-6-(1H-pyrazol-4-yl)pyridin-4-yl)-4-methylphenyl]-3-(2,2,2-trifluoroethyl)pyrrolidine-1-carboxamide (168 mg, 59%) as an off-white solid. MS ESI calculated for C₂₅H₂₉F₃N₆O₂[M+H]⁺, 503.23; found 503.30. ¹H NMR (400 MHz, DMSO-d₆) δ 12.91 (s, 1H), 8.19-8.15 (m, 2H), 7.98 (s, 1H), 7.48-7.40 (m, 2H), 7.15-7.13 (m, 1H), 6.75 (s, 1H), 6.23-6.21 (m, 2H), 4.89-4.86 (m, 1H), 4.03-4.00 (m, 1H), 3.70-3.65 (m, 1H), 3.58-3.51 (m, 2H), 3.38-3.28 (m, 3H), 3.05-3.00 (m, 1H), 2.53-2.50 (m, 2H), 2.20 (s, 3H), 2.15-2.05 (m, 1H), 1.75-1.55 (m, 1H), 1.20 (s, 3H). ¹⁹F NMR (282 MHz, DMSO-d₆) δ-63.36 (3F).

### Example 81: (3S)-N-[3-(6-[[(2R)-1-hydroxypropan-2-yl]amino]-[2,4′-bipyridin]-4-yl)-4-methylphenyl]-3-(2,2,2-trifluoroethyl)pyrrolidine-1-carboxamide

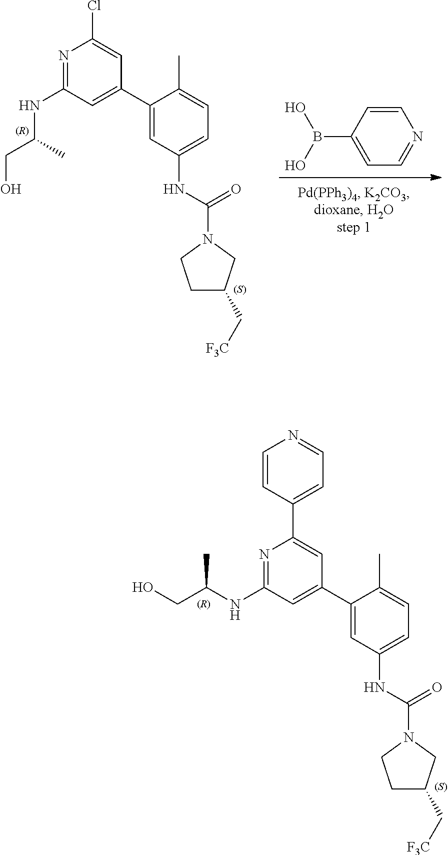

To a stirred mixture of (3S)-N-[3-(2-chloro-6-[[(2R)-1-hydroxypropan-2-yl]amino]pyridin-4-yl)-4-methylphenyl]-3-(2,2,2-trifluoroethyl)pyrrolidine-1-carboxamide (150 mg, 0.319 mmol), pyridin-4-ylboronic acid (58 mg, 0.478 mmol) and K₂CO₃ (88 mg, 0.637 mmol) in dioxane (2 mL) and H₂O (0.5 mL) was added Pd(PPh₃)₄(37 mg, 0.032 mmol) at room temperature. The reaction mixture was stirred for 2 h at 80° C. under nitrogen atmosphere. The resulting mixture was diluted with water (50 mL). The resulting mixture was extracted with EtOAc (3×50 mL). The combined organic layers were washed with brine (2×50 mL), dried over anhydrous Na₂SO₄.

After filtration, the filtrate was concentrated under reduced pressure. The residue was purified by silica gel column chromatography, eluted with (EA/PE/EtOH=12/3/1). The fractions containing the desired product were collected and concentrated under reduced pressure. The residue was purified by reverse phase chromatography, eluted with CH₃CN in water (with 10 mmol/mL NH₄HCO₃). The fractions containing the desired product were collected and concentrated under reduced pressure to afford (3S)-N-[3-(6-[[(2R)-1-hydroxypropan-2-yl]amino]-[2,4′-bipyridin]-4-yl)-4-methylphenyl]-3-(2,2,2-trifluoroethyl)pyrrolidine-1-carboxamide (141 mg, 86%) as a white solid. MS ESI calculated for C₂₇H₃₀F₃N₅O₂ [M+H]⁺, 514.24, found 514.20; ¹H NMR (400 MHz, DMSO-d₆) δ 8.67-8.61 (m, 2H), 8.18 (s, 1H), 8.06-8.00 (m, 2H), 7.52-7.43 (m, 2H), 7.20-7.13 (m, 2H), 6.60 (d, J=7.6 Hz, 1H), 6.51 (d, J=1.1 Hz, 1H), 4.76 (t, J=5.6 Hz, 1H), 4.16 (p, J=6.4 Hz, 1H), 3.67 (dd, J=10.2, 6.9 Hz, 1H), 3.63-3.48 (m, 2H), 3.40 (dt, J=10.4, 6.0 Hz, 1H), 3.32-3.27 (m, 1H), 3.03 (t, J=9.4 Hz, 1H), 2.48-2.36 (m, 3H), 2.22 (s, 3H), 2.09 (h, J=6.3 Hz, 1H), 1.66 (p, J=9.8 Hz, 1H), 1.21 (d, J=6.6 Hz, 3H). ¹⁹F NMR (282 MHz, DMSO-d₆) δ-63.37 (3F).

### Example 82: (3S)-N-[3-(2′-amino-6-[[(2R)-1-hydroxypropan-2-yl]amino]-[2,4′-bipyridin]-4-yl)-4-methylphenyl]-3-(2,2,2-trifluoroethyl)pyrrolidine-1-carboxamide

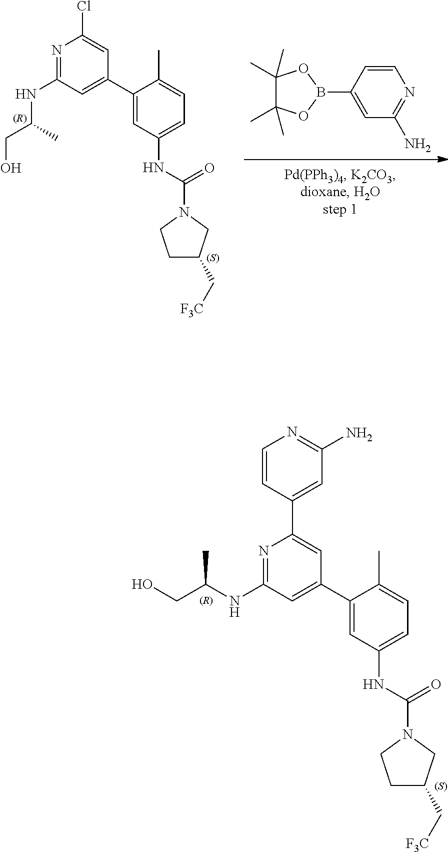

To a stirred mixture of (3S)-N-[3-(2-chloro-6-[[(2R)-1-hydroxypropan-2-yl]amino]pyridin-4-yl)-4-methylphenyl]-3-(2,2,2-trifluoroethyl)pyrrolidine-1-carboxamide (150 mg, 0.319 mmol), 4-(4,4,5,5-tetramethyl-1,3,2-dioxaborolan-2-yl)pyridin-2-amine (84 mg, 0.382 mmol) and K₂CO₃(88 mg, 0.637 mmol) in dioxane (2 ml) and H₂O (0.5 ml) was added Pd(PPh₃)₄(37 mg, 0.032 mmol). The reaction mixture was stirred for 2 h at 80° C. under N₂ atmosphere. The resulting mixture was diluted with water (40 mL). The resulting mixture was extracted with EtOAc (3×40 mL). The combined organic layers were washed with brine (3×40 mL), dried over anhydrous Na₂SO₄. After filtration, the filtrate was concentrated under reduced pressure. The residue was purified by silica gel column chromatography, eluted with (EA/PE/EtOH=12/3/1). The fractions containing the desired product were collected and concentrated under reduced pressure. The residue was purified by reverse phase chromatography, eluted with CH₃CN in water (with 10 mmol/mL NH₄HCO₃). The fractions containing the desired product were collected and concentrated under reduced pressure to afford (3S)-N-[3-(2′-amino-6-[[(2R)-1-hydroxypropan-2-yl]amino]-[2,4′-bipyridin]-4-yl)-4-methylphenyl]-3-(2,2,2-trifluoroethyl)pyrrolidine-1-carboxamide (101 mg, 58%) as a white solid. MS ESI calculated for C₂₇H₃₁F₃N₆O₂[M+H]⁺, 529.25, found 529.30; ¹H NMR (400 MHz, DMSO-d₆) δ 8.17 (s, 1H), 7.95 (dd, J=5.2, 0.8 Hz, 1H), 7.51-7.42 (m, 2H), 7.19-7.07 (m, 3H), 6.90 (d, J=1.2 Hz, 1H), 6.51-6.44 (m, 2H), 5.91 (s, 2H), 4.75 (t, J=5.5 Hz, 1H), 4.14 (p, J=6.4 Hz, 1H), 3.67 (dd, J=10.2, 6.9 Hz, 1H), 3.59-3.50 (m, 2H), 3.40 (dt, J=10.4, 5.9 Hz, 1H), 3.29 (d, J=6.7 Hz, 1H), 3.03 (t, J=9.4 Hz, 1H), 2.49-2.36 (m, 3H), 2.21 (s, 3H), 2.13-2.05 (m, 1H), 1.66 (p, J=10.1 Hz, 1H), 1.20 (d, J=6.6 Hz, 3H).

### Example 83: (3S)-N-[3-(2′-cyano-6-[[(2R)-1-hydroxypropan-2-yl]amino]-[2,4′-bipyridin]-4-yl)-4-methylphenyl]-3-(2,2,2-trifluoroethyl)pyrrolidine-1-carboxamide

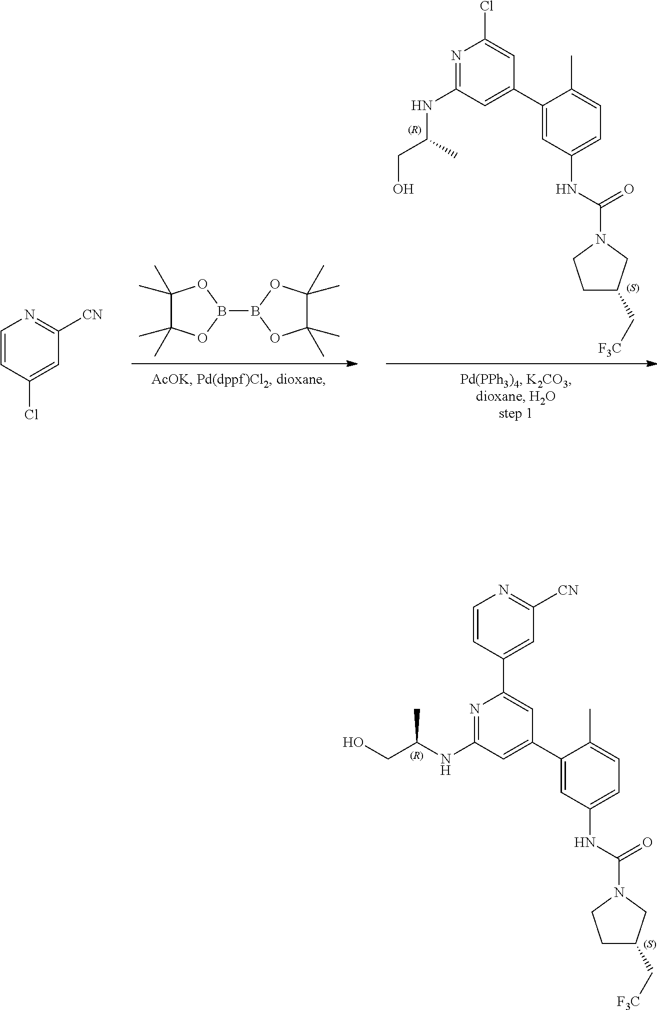

To a solution of 4-chloropyridine-2-carbonitrile (0.50 g, 3.61 mmol) and bis(pinacolato)diboron (1.83 g, 7.21 mmol) in 1,4-dioxane (10 mL) were added AcOK (1.06 g, 10.83 mmol) and Pd(dppf)Cl₂ (0.53 g, 0.72 mmol). After stirring for 2 h at 100° C. under a nitrogen atmosphere. To above solution were added (3S)-N-[3-(2-chloro-6-[[(2R)-1-hydroxypropan-2-yl]amino]pyridin-4-yl)-4-methylphenyl]-3-(2,2,2-trifluoroethyl)pyrrolidine-1-carboxamide (0.30 g, 0.65 mmol), 4-(4,4,5,5-tetramethyl-1,3,2-dioxaborolan-2-yl)pyridine-2-carbonitrile (0.30 g, 1.30 mmol), H₂O (0.4 mL), Pd(PPh₃)₄(0.07 mg, 0.06 mmol) and K₂CO₃ (0.18 g, 1.30 mmol) at room temperature under nitrogen atmosphere. After stirring for 2 h at 80° C. The resulting mixture was concentrated under reduced pressure. The residue was purified by Prep-TLC, eluted with (PE/EtOAc/EtOH=12/3/1). The residue was purified by reverse phase flash chromatography with the following conditions: Column: Spherical C18, 20-40 um, 120 g; Mobile Phase A: Water (plus 5 mmol/mL NH₄HCO₃); Mobile Phase B: CH₃CN; Flow rate: 80 mL/min; Gradient: 38%-60% B gradient in 25 min; Detector: 220 nm. The fractions containing the desired product were collected at 54% B to afford (3S)-N-[3-(2-cyano-6-[[(2R)-1-hydroxypropan-2-yl]amino]-[2,4-bipyridin]-4-yl)-4-methylphenyl]-3-(2,2,2-trifluoroethyl)pyrrolidine-1-carboxamide (0.12 g, 33%) as a light yellow solid. MS ESI calculated for C₂₈H₂₉F₃N₆O₂[M+H]⁺, 539.23, found 539.10. ¹H NMR (400 MHz, DMSO-d₆) δ 8.81 (dd, J=5.2, 0.8 Hz, 1H), 8.67 (dd, J=1.8, 0.8 Hz, 1H), 8.40 (dd, J=5.2, 1.8 Hz, 1H), 8.19 (s, 1H), 7.52-7.44 (m, 2H), 7.34 (d, J=1.1 Hz, 1H), 7.18 (d, J=8.2 Hz, 1H), 6.71 (d, J=7.6 Hz, 1H), 6.57 (d, J=1.1 Hz, 1H), 4.76 (t, J=5.6 Hz, 1H), 4.18 (p, J=6.4 Hz, 1H), 3.68 (dd, J=10.2, 6.9 Hz, 1H), 3.63-3.48 (m, 2H), 3.39 (dt, J=10.4, 6.1 Hz, 1H), 3.30 (dd, J=10.0, 6.7 Hz, 1H), 3.03 (t, J=9.4 Hz, 1H), 2.51-2.34 (m, 3H), 2.22 (s, 3H), 2.09 (d, J=10.3 Hz, 1H), 1.66 (p, J=9.7 Hz, 1H), 1.22 (d, J=6.6 Hz, 3H). ¹⁹F NMR (376 MHz, DMSO-d₆) δ-63.367 (3F).

### Examples 84 and 85: (3S)-N-[3-(2-[[(2R)-1-hydroxypropan-2-yl]amino]-6-[(1R,6S)-3-oxabicyclo[4.1.0]heptan-6-yl]pyridin-4-yl)-4-methylphenyl]-3-(2,2,2-trifluoroethyl)pyrrolidine-1-carboxamide, (3S)-N-[3-(2-[[(2R)-1-hydroxypropan-2-yl]amino]-6-[(1S,6R)-3-oxabicyclo[4.1.0]heptan-6-yl]pyridin-4-yl)-4-methylphenyl]-3-(2,2,2-trifluoroethyl)pyrrolidine-1-carboxamide

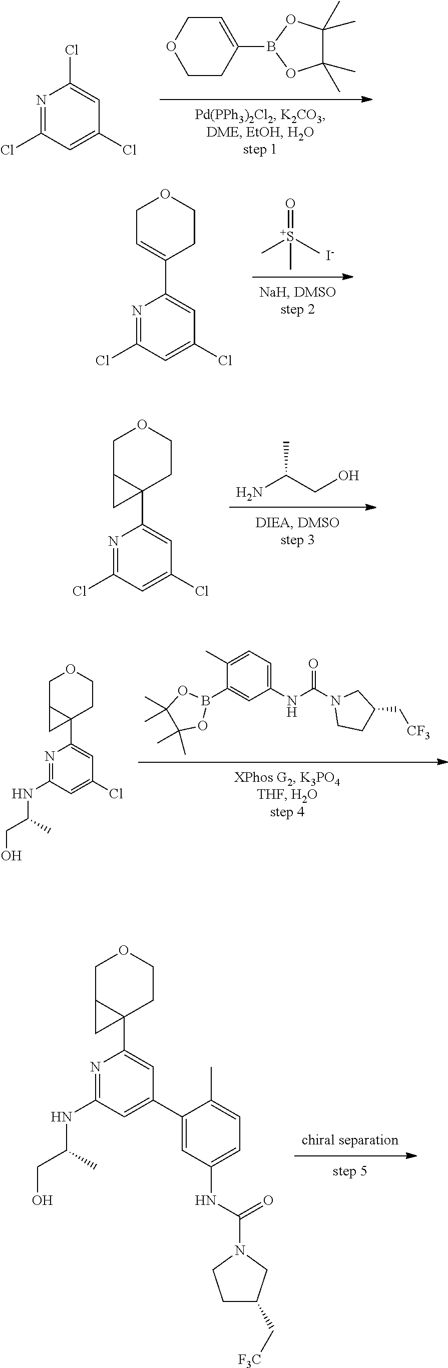

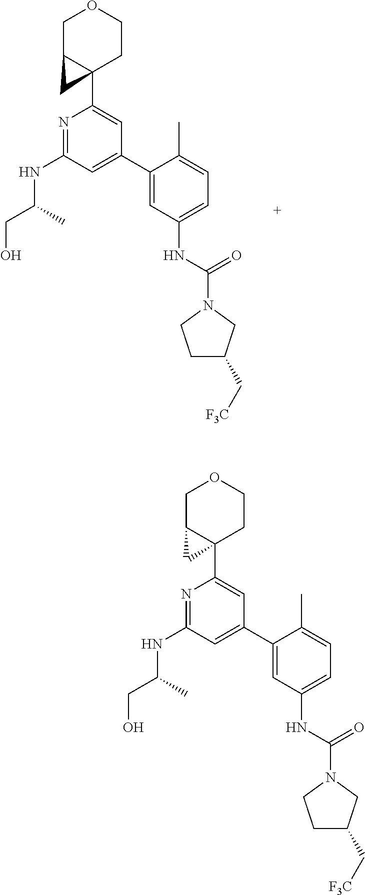 [SMILES: Cc1ccc(NC(=O)N2CC[C@@H](CC(F)(F)F)C2)cc1-c1cc(N[C@H](C)CO)nc([C@@]23CCOC[C@@H]2C3)c1 ; Cc1ccc(NC(=O)N2CC[C@@H](CC(F)(F)F)C2)cc1-c1cc(N[C@H](C)CO)nc([C@]23CCOC[C@H]2C3)c1]

Preparation 84A: 2,4-dichloro-6-(3,6-dihydro-2H-pyran-4-yl)pyridine

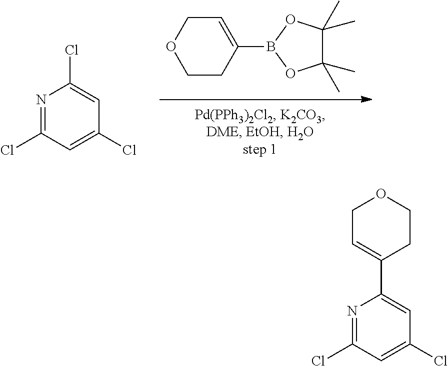

To a stirred solution of 2,4,6-trichloropyridine (5.00 g, 27.41 mmol), 2-(3,6-dihydro-2H-pyran-4-yl)-4,4,5,5-tetramethyl-1,3,2-dioxaborolane (5.76 g, 27.41 mmol) and K₂CO₃ (7.58 g, 54.82 mmol) in DME/EtOH/H₂O=2/1/2 (50 mL) was added Pd(PPh₃)₂Cl₂ (0.96 g, 1.37 mmol) at room temperature under nitrogen atmosphere. The resulting mixture was stirred for 16 h at 80° C. under nitrogen atmosphere. The reaction was quenched by the addition of water (100 mL). The resulting mixture was extracted with EtOAc (3×50 mL). The combined organic layers were washed with saturated brine (3×50 mL), dried over anhydrous Na₂SO₄. After filtration, the filtrate was concentrated under reduced pressure. The residue was purified by silica gel column chromatography, eluted with PE/EtOAc (2/1) to afford 2,4-dichloro-6-(3,6-dihydro-2H-pyran-4-yl)pyridine (2.77 g, 44%) as an off-white solid. MS ESI calculated for C₁₀H₉Cl₂NO [M+H]⁺, 230.01, found 229.90. ¹H NMR (300 MHz, chloroform-d) δ 7.26-7.25 (m, 1H), 7.23-7.22 (m, 1H), 6.86-6.82 (m, 1H), 4.40-4.37 (m, 2H), 3.95-3.91 (m, 2H), 2.60-2.54 (m, 2H).

Preparation 84B: 2,4-dichloro-6-[3-oxabicyclo[4.1.0]heptan-6-yl]pyridine

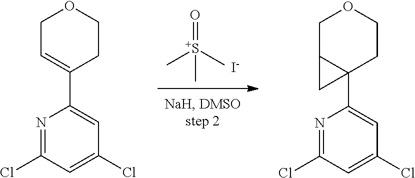

To a stirred solution of trimethylsulfoxonium iodide (7.65 g, 34.77 mmol) in DMSO (20 mL) was added NaH (1.40 g, 35.03 mmol, 60%) in portions at 0° C. under nitrogen atmosphere. The resulting mixture was stirred for 30 min at room temperature under nitrogen atmosphere. To the above mixture was added 2,4-dichloro-6-(3,6-dihydro-2H-pyran-4-yl)pyridine (2.00 g, 8.69 mmol) at room temperature. The resulting mixture was stirred for additional 16 h at 60° C. The reaction was quenched by the addition of water (100 mL). The resulting mixture was extracted with EtOAc (3×50 mL). The combined organic layers were washed with saturated brine (3×50 mL), dried over anhydrous Na₂SO₄. After filtration, the filtrate was concentrated under reduced pressure. The residue was purified by silica gel column chromatography, eluted with PE/EtOAc (4/1) to afford 2,4-dichloro-6-[3-oxabicyclo[4.1.0]heptan-6-yl]pyridine (1.7 g, 80%) as an off-white solid. MS ESI calculated for C₁₁Hn₁₁Cl₂NO [M+H]⁺. 244.02, found 243.95. ¹H NMR (300 MHz, chloroform-d) δ 7.14-7.13 (m, 2H), 4.04-3.93 (m, 2H), 3.68-3.61 (m, 1H), 3.50-3.41 (m, 1H), 2.49-2.41 (m, 1H), 2.15-2.06 (m, 1H), 1.82-1.75 (m. 1H), 1.36-1.32 (m, 1H), 1.15-1.11 (m, 1H).

Preparation 84C: (2R)-2-[(4-chloro-6-[3-oxabicyclo[4.1.0]heptan-6-yl]pyridin-2-yl)amino]propan-1-ol

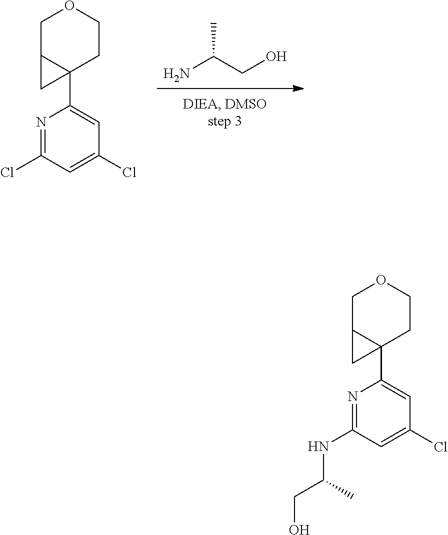

To a stirred solution of 2,4-dichloro-6-[3-oxabicyclo[4.1.0]heptan-6-yl]pyridine (1.40 g, 5.73 mmol) and (R)-(−)-2-amino-1-propanol (0.86 g, 11.47 mmol) in DMSO (15 mL) was added DIEA (1.48 g, 11.47 mmol) dropwise at room temperature under nitrogen atmosphere. The resulting mixture was stirred for 16 h at 130° C. under nitrogen atmosphere. The reaction was quenched by the addition of water (100 mL). The resulting mixture was extracted with EtOAc (3×50 mL). The combined organic layers were washed with saturated brine (3×50 mL), dried over anhydrous Na₂SO₄. After filtration, the filtrate was concentrated under reduced pressure. The residue was purified by reverse flash chromatography with the following conditions: column, C₁₈ silica gel; mobile phase, CH₃CN in water (0.1% TFA), 5% to 95% gradient in 40 min; detector, UV 254 nm; to afford (2R)-2-[(4-chloro-6-[3-oxabicyclo[4.1.0]heptan-6-yl]pyridin-2-yl)amino]propan-1-ol (0.21 g, 13%) as an off-white solid. MS ESI calculated for C₁₄H₁₉ClN₂O₂ [M+H]⁺. 283.11, found 283.20. ¹H NMR (300 MHz, DMSO-d₆) δ 6.44-6.42 (m, 2H), 6.31 (m, 1H), 4.65 (s, 1H), 3.91-3.74 (m, 3H), 3.50-3.36 (m, 3H), 3.29-3.22 (m, 1H), 2.42-2.34 (m, 1H), 1.92-1.83 (m, 1H), 1.67-1.61 (m, 1H), 1.22-1.19 (m, 1H), 1.09-1.07 (m, 3H), 0.90-0.85 (m, 1H).

Preparation 84D: (3S)-N-[3-(2-[[(2R)-1-hydroxypropan-2-yl]amino]-6-[3-oxabicyclo[4.1.0]heptan-6-yl]pyridin-4-yl)-4-methylphenyl]-3-(2,2,2-trifluoroethyl)pyrrolidine-1-carboxamide

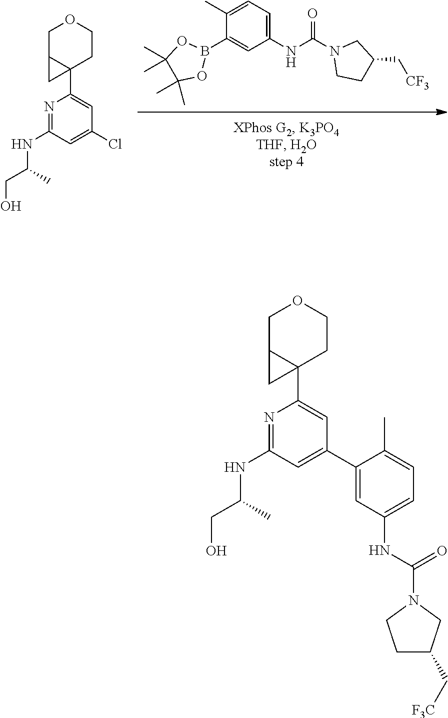 [SMILES: C[C@H](CO)Nc1cc(Cl)cc(C23CCOCC2C3)n1 ; Cc1ccc(NC(=O)N2CC[C@@H](CC(F)(F)F)C2)cc1B1OC(C)(C)C(C)(C)O1 ; Cc1ccc(NC(=O)N2CC[C@@H](CC(F)(F)F)C2)cc1-c1cc(N[C@H](C)CO)nc(C23CCOCC2C3)c1]

To a stirred solution of (2R)-2-[(4-chloro-6-[3-oxabicyclo[4.1.0]heptan-6-yl]pyridin-2-yl)amino]propan-1-ol (310 mg, 1.096 mmol), (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (452 mg, 1.096 mmol) and K₃PO₄ (465 mg, 2.193 mmol) in THF/H₂O=10/1 (4 mL) was added XPhos Pd G2 (86 mg, 0.110 mmol) at room temperature under nitrogen atmosphere. The resulting mixture was stirred for 16 h at 80° C. under nitrogen atmosphere. The reaction was quenched by the addition of water (100 mL). The resulting mixture was extracted with EtOAc (3×50 mL). The combined organic layers were washed with saturated brine (3×50 mL), dried over anhydrous Na₂SO₄. After filtration, the filtrate was concentrated under reduced pressure. The residue was purified by silica gel column chromatography, eluted with (PE/EA/EtOH=8/3:1) to afford (3S)-N-[3-(2-[[(2R)-1-hydroxypropan-2-yl]amino]-6-[3-oxabicyclo[4.1.0]heptan-6-yl]pyridin-4-yl)-4-methylphenyl]-3-(2,2,2-trifluoroethyl)pyrrolidine-1-carboxamide (350 mg, 60%) as a light yellow solid. MS ESI calculated for C₂₈H₃₅F₃N₄O₃[M+H]⁺. 533.27, found 533.15. ¹H NMR (300 MHz, DMSO-d₆) δ 8.14 (s, 1H), 7.46-7.43 (m, 1H), 7.37-7.36 (m, 1H), 7.14-7.11 (m, 1H), 6.34 (s, 1H), 6.19 (m, 2H), 4.70 (m, 1H), 3.97-3.92 (m, 2H), 3.82-3.78 (m, 1H), 3.71-3.65 (m, 1H), 3.57-3.47 (m, 3H), 3.42-3.35 (m, 2H), 3.31-3.28 (m, 1H), 3.06-3.00 (m, 1H), 2.24 (m, 3H), 2.17-2.10 (m, 4H), 2.00-1.89 (m, 1H), 1.74-1.63 (m, 3H), 1.28-1.27 (m, 1H), 1.15-1.12 (m, 3H), 0.93-0.88 (m, 1H). ¹⁹F NMR (282 MHz, DMSO-d₆) δ-63.36 (3F).

### Examples 84 and 85: (3S)-N-[3-(2-[[(2R)-1-hydroxypropan-2-yl]amino]-6-[(1R,6S)-3-oxabicyclo[4.1.0]heptan-6-yl]pyridin-4-yl)-4-methylphenyl]-3-(2,2,2-trifluoroethyl)pyrrolidine-1-carboxamide, (3S)-N-[3-(2-[[(2R)-1-hydroxypropan-2-yl]amino]-6-[(1S,6R)-3-oxabicyclo[4.1.0]heptan-6-yl]pyridin-4-yl)-4-methylphenyl]-3-(2,2,2-trifluoroethyl)pyrrolidine-1-carboxamide

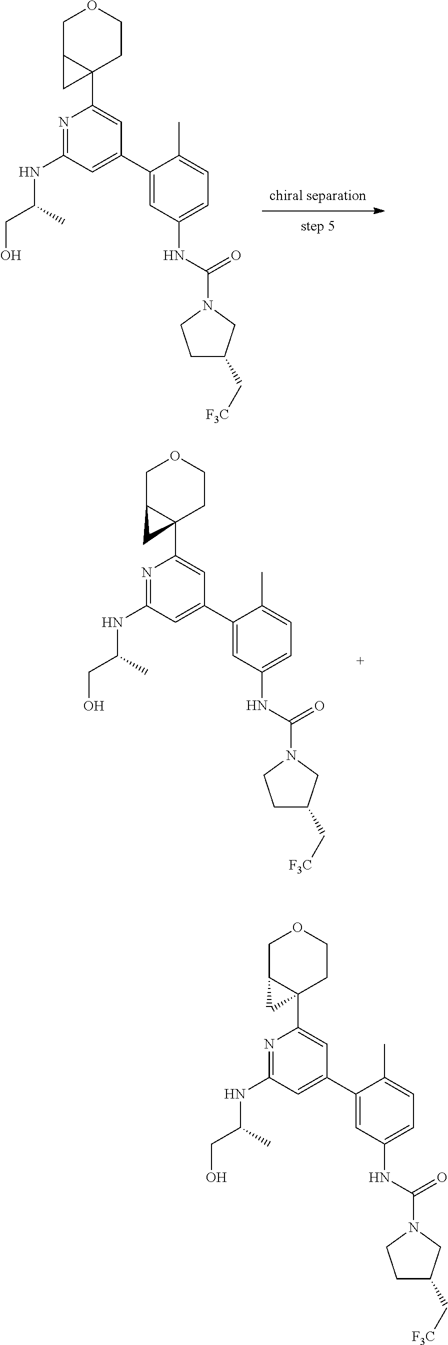 [SMILES: Cc1ccc(NC(=O)N2CC[C@@H](CC(F)(F)F)C2)cc1-c1cc(N[C@H](C)CO)nc(C23CCOCC2C3)c1 ; Cc1ccc(NC(=O)N2CC[C@@H](CC(F)(F)F)C2)cc1-c1cc(N[C@H](C)CO)nc([C@@]23CCOC[C@@H]2C3)c1 ; Cc1ccc(NC(=O)N2CC[C@@H](CC(F)(F)F)C2)cc1-c1cc(N[C@H](C)CO)nc([C@]23CCOC[C@H]2C3)c1]

The crude product ((3S)-N-[3-(2-[[(2R)-1-hydroxypropan-2-yl]amino]-6-[3-oxabicyclo[4.1.0]heptan-6-yl]pyridin-4-yl)-4-methylphenyl]-3-(2,2,2-trifluoroethyl)pyrrolidine-1-carboxamide (350 mg, 0.657 mmol) was resolved by Chiral-Prep-HPLC with the following conditions (Column: CHIRAL ART Cellulose-SB, 2*25 cm, 5 um; Mobile Phase A:Hex (0.5% 2M NH₃-MeOH)-HPLC, Mobile Phase B:EtOH-HPLC; Flow rate:20 mL/min; Gradient:20 B to 20 B in 19 min; 254/220 nm). The first peak afforded (3S)-N-[3-(2-[[(2R)-1-hydroxypropan-2-yl]amino]-6-[(1R,6S)-3-oxabicyclo[4.1.0]heptan-6-yl]pyridin-4-yl)-4-methylphenyl]-3-(2,2,2-trifluoroethyl)pyrrolidine-1-carboxamide (139 mg, 40%) as an off-white solid. MS ESI calculated for C₂₈H₃₅F₃N₄O₃[M+H]⁺. 533.27, found 533.20. ¹H NMR (400 MHz, DMSO-d₆) δ 8.12 (s, 1H), 7.45-7.35 (m, 2H), 7.12-7.10 (m, 1H), 6.33-6.32 (m, 1H), 6.17-6.14 (m, 2H), 4.69-4.65 (m, 1H), 3.95-3.90 (m, 2H), 3.80-3.77 (m, 1H), 3.69-3.63 (m, 1H), 3.52-3.45 (m, 3H), 3.38-3.34 (m, 2H), 3.31-3.25 (m, 1H), 3.04-2.99 (m, 1H), 2.46-2.40 (m, 3H), 2.15-2.05 (m, 4H), 1.96-1.87 (m, 1H), 1.73-1.63 (m, 3H), 1.26-1.22 (m, 1H), 1.13-1.10 (m, 3H), 0.90-0.88 (m, 1H). ¹⁹F NMR (376 MHz, DMSO-d₆) δ-63.36 (3F).

The second peak afforded (3S)-N-[3-(2-[[(2R)-1-hydroxypropan-2-yl]amino]-6-[(1S,6R)-3-oxabicyclo[4.1.0]heptan-6-yl]pyridin-4-yl)-4-methylphenyl]-3-(2,2,2-trifluoroethyl)pyrrolidine-1-carboxamide (150 mg, 43%) as an off-white solid. MS ESI calculated for C₂₈H₃₅F₃N₄O₃[M+H]⁺. 533.27, found 533.20. ¹H NMR (400 MHz, DMSO-d₆) δ 8.12 (s, 1H), 7.44-7.35 (m, 2H), 7.12-7.10 (m, 1H), 6.33 (m, 1H), 6.17-6.15 (m, 2H), 4.70-4.66 (m, 1H), 3.95-3.90 (m, 2H), 3.80-3.77 (m, 1H), 3.69-3.63 (m, 1H), 3.52-3.45 (m, 3H), 3.38-3.35 (m, 2H), 3.31-3.28 (m, 1H), 3.04-2.99 (m, 1H), 2.46-2.40 (m, 3H), 2.15-2.07 (m, 4H), 1.96-1.87 (m, 1H), 1.73-1.63 (m, 3H), 1.26-1.22 (m, 1H), 1.13-1.11 (m, 3H), 0.90-0.87 (m, 1H). ¹⁹F NMR (376 MHz, DMSO-d₆) δ-63.35 (3F).

### Example 86: (3S)-N-[3-(2-[[(2R)-2,3-dihydroxypropyl]amino]-6-(morpholin-4-yl)pyridin-4-yl)-4-methylphenyl]-3-(2,2,2-trifluoroethyl)pyrrolidine-1-carboxamide

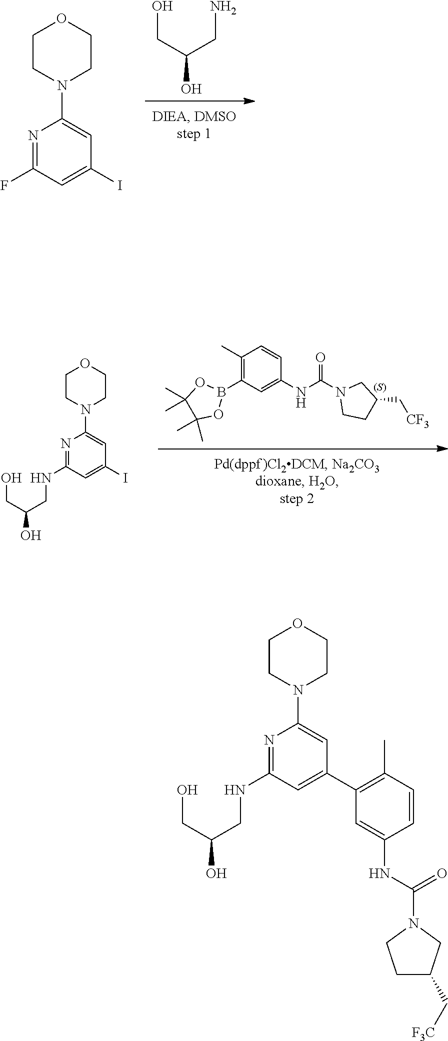

Preparation 86A: (2R)-3-[[4-iodo-6-(morpholin-4-yl)pyridin-2-yl]amino]propane-1,2-diol

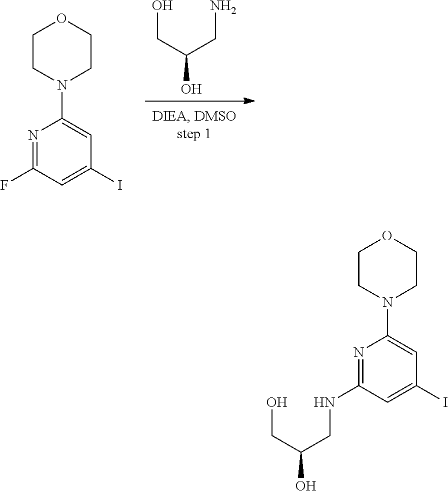 [SMILES: Fc1cc(I)cc(N2CCOCC2)n1 ; NC[C@@H](O)CO ; OC[C@H](O)CNc1cc(I)cc(N2CCOCC2)n1]

To a stirred solution of 4-(6-fluoro-4-iodopyridin-2-yl)morpholine (600 mg, 1.947 mmol) and (2R)-3-aminopropane-1,2-diol (266 mg, 2.921 mmol) in DMSO (6 mL) was added DIEA (1 mL, 7.874 mmol) under nitrogen atmosphere. The resulting mixture was stirred for 16 h at 80° C. under nitrogen atmosphere. The resulting mixture was concentrated under reduced pressure. The resulting mixture was extracted with EtOAc (3×50 mL). The combined organic layers were washed with brine (3×50 mL), dried over anhydrous Na₂SO₄. After filtration, the filtrate was concentrated under reduced pressure. The residue was purified by silica gel column chromatography, eluted with (EA/PE/EtOH=12/3/1) to afford (2R)-3-[[4-iodo-6-(morpholin-4-yl)pyridin-2-yl]amino]propane-1,2-diol (350 mg, 47%) as a light yellow solid. MS ESI calculated for C₁₂H₁₈IN₃O₃ [M+H]⁺, 380.19, found 380.00. ¹H NMR (400 MHz, chloroform-d) δ 6.258-6.21 (m, 2H), 4.70 (s, 1H), 3.89-3.80 (m, 1H), 3.80-3.74 (m, 4H), 3.67-3.55 (m, 2H), 3.67-3.55 (m, 2H), 3.42-3.36 (m, 4H).

### Example 86: (3S)-N-[3-(2-[[(2R)-2,3-dihydroxypropyl]amino]-6-(morpholin-4-yl)pyridin-4-yl)-4-methylphenyl]-3-(2,2,2-trifluoroethyl)pyrrolidine-1-carboxamide

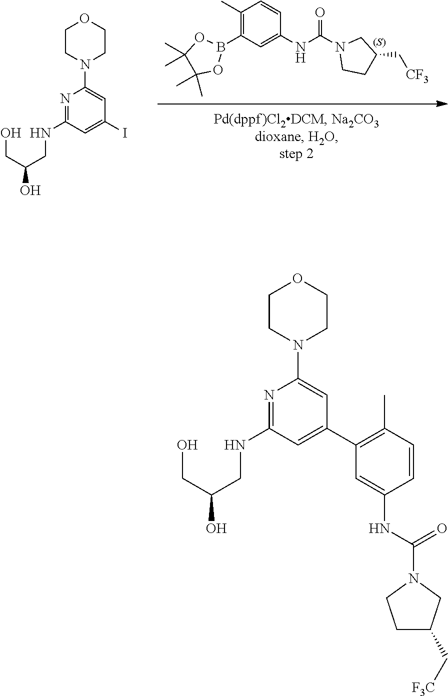

To a stirred solution of (2R)-3-[[4-iodo-6-(morpholin-4-yl)pyridin-2-yl]amino]propane-1,2-diol (200 mg, 0.527 mmol) and (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (217 mg, 0.527 mmol) in 1,4-dioxane (2 mL) and H₂O (0.4 mL) were added Na₂CO₃ (167 mg, 1.582 mmol) and Pd(dppf)Cl₂·CH₂Cl₂ (43 mg, 0.053 mmol). The resulting mixture was stirred for 16 h at 60° C. under nitrogen atmosphere. The mixture was allowed to cool down to room temperature. The reaction was quenched by the addition of water (50 mL) at room temperature. The resulting mixture was extracted with EtOAc (3×50 mL). The combined organic layers were washed with brine (3×50 mL), dried over anhydrous Na₂SO₄. After filtration, the filtrate was concentrated under reduced pressure. The residue was purified by silica gel column chromatography, eluted with (EtOAc/PE/EtOH=12/3/1) to afford (3S)-N-[3-(2-[[(2R)-2,3-dihydroxypropyl]amino]-6-(morpholin-4-yl)pyridin-4-yl)-4-methylphenyl]-3-(2,2,2-trifluoroethyl)pyrrolidine-1-carboxamide (186 mg, 66%) as an off-white solid. MS ESI calculated for C₂₆H₃₄F₃N₅O₄[M+H]⁺, 537.26, found 537.26. ¹H NMR (400 MHz, DMSO-d₆) δ 8.11 (s, 1H), 7.45-7.38 (m, 1H), 7.33 (d, J=2.4 Hz, 1H), 7.09 (d, J=8.4 Hz, 1H), 6.17 (d, J=6.1 Hz, 1H), 5.81-5.74 (m, 2H), 4.80 (d, J=4.8 Hz, 1H), 4.53 (t, J=5.7 Hz, 1H), 3.71-3.58 (m, 6H), 3.57-3.48 (m, 1H), 3.44-3.25 (m, 8H), 3.19-3.08 (m, 1H), 3.02 (t, J=9.4 Hz, 1H), 2.51-2.34 (m, 3H), 2.16 (s, 3H), 2.08 (d, J=6.9 Hz, 1H), 1.73-1.58 (m, 1H). ¹⁹F NMR (376 MHz, DMSO-d₆) δ-63.24 (3F).

### Example 87: (3S)-N-[3-(2-[[(2S)-2,3-dihydroxypropyl]amino]-6-(morpholin-4-yl)pyridin-4-yl)-4-methylphenyl]-3-(2,2,2-trifluoroethyl)pyrrolidine-1-carboxamide

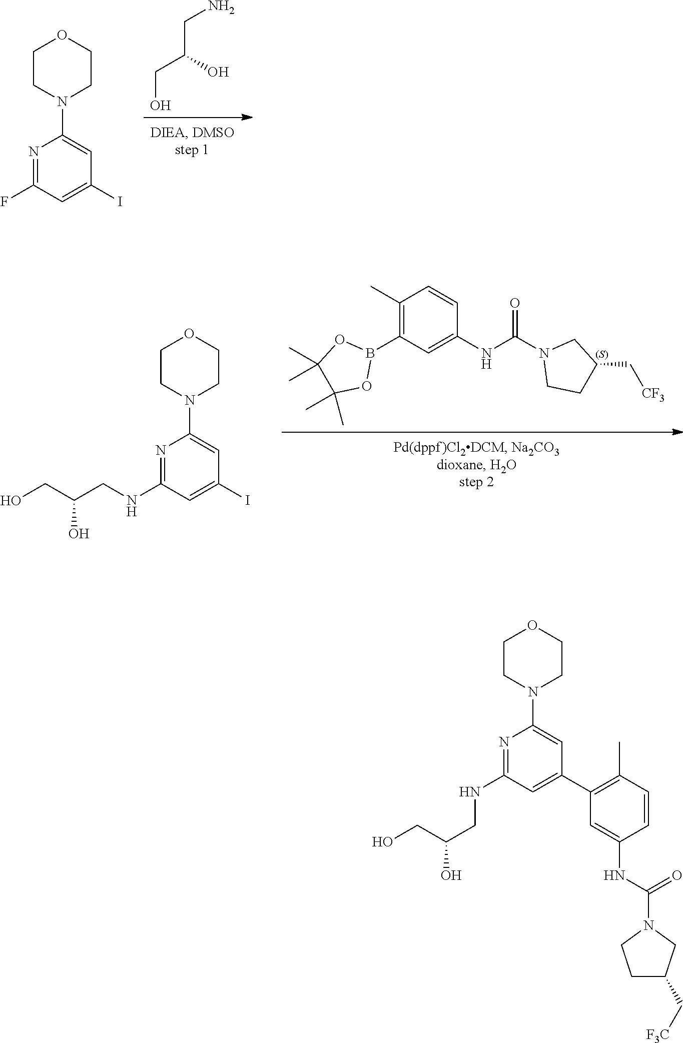

Preparation 87A: (2S)-3-[[4-iodo-6-(morpholin-4-yl)pyridin-2-yl]amino]propane-1,2-diol

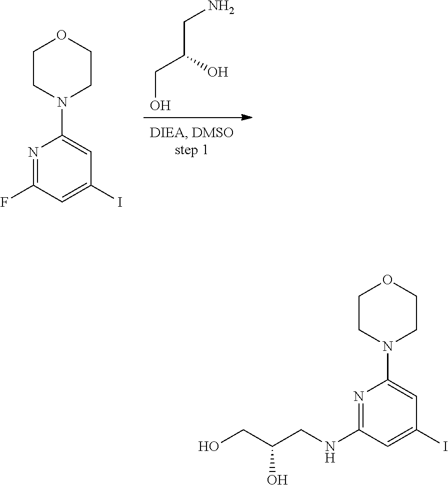 [SMILES: Fc1cc(I)cc(N2CCOCC2)n1 ; NC[C@H](O)CO ; OC[C@@H](O)CNc1cc(I)cc(N2CCOCC2)n1]

To a stirred solution of 4-(6-fluoro-4-iodopyridin-2-yl)morpholine (600 mg, 1.947 mmol) and 3-aminopropane-1,2-diol (266 mg, 2.921 mmol) in DMSO (6 mL) was added DIEA (1 mL, 5.841 mmol). The resulting mixture was stirred for 16 h at 80° C. The resulting mixture was extracted with EtOAc (3×50 mL). The combined organic layers were washed with brine (3×50 mL), dried over anhydrous Na₂SO₄. After filtration, the filtrate was concentrated under reduced pressure. The residue was purified by silica gel column chromatography, eluted with PE/EtOAc (1/1) to afford (2S)-3-[[4-iodo-6-(morpholin-4-yl)pyridin-2-yl]amino]propane-1,2-diol (400 mg, 54%) as a light yellow solid. MS ESI calculated for C₁₂H₁₈IN₃O₃ [M+H]⁺, 380.19, found 380.05. ¹H NMR (400 MHz, chloroform-d) δ 6.30-6.23 (m, 2H), 4.72 (t, J=6.1 Hz, 1H), 3.91-3.82 (m, 1H), 3.82-3.75 (m, 4H), 3.69-3.57 (m, 2H), 3.57-3.45 (m, 2H), 3.45-3.38 (m, 4H).

### Example 87: (3S)-N-[3-(2-[[(2S)-2,3-dihydroxypropyl]amino]-6-(morpholin-4-yl)pyridin-4-yl)-4-methylphenyl]-3-(2,2,2-trifluoroethyl)pyrrolidine-1-carboxamide

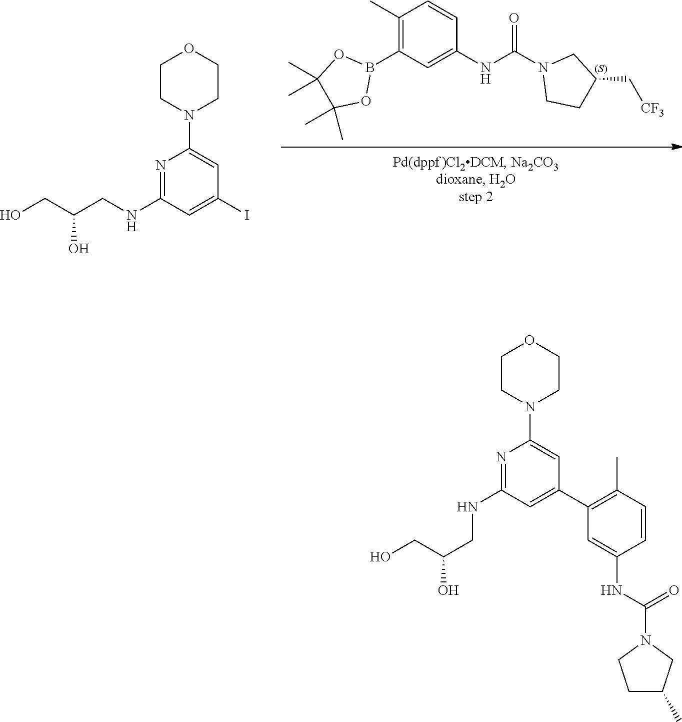

To a stirred solution of (2S)-3-[[4-iodo-6-(morpholin-4-yl)pyridin-2-yl]amino]propane-1,2-diol (200 mg, 0.527 mmol) and (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (217 mg, 0.527 mmol) in 1,4-dioxane (2 mL) and H₂O (0.4 mL) were added Na₂CO₃ (168 mg, 1.582 mmol) and Pd(dppf)Cl₂·CH₂Cl₂ (43 mg, 0.053 mmol) at room temperature under nitrogen atmosphere. The resulting mixture was stirred for 16 h at 60° C. under nitrogen atmosphere. The mixture was allowed to cool down to room temperature. The reaction was quenched by the addition of water (50 mL) at room temperature. The resulting mixture was extracted with EtOAc (3×50 mL). The combined organic layers were washed with brine (3×50 mL), dried over anhydrous Na₂SO₄. After filtration, the filtrate was concentrated under reduced pressure. The residue was purified by silica gel column chromatography, eluted with (PE/EtOAc/EtOH=12/3/1) to afford (3S)-N-[3-(2-[[(2S)-2,3-dihydroxypropyl]amino]-6-(morpholin-4-yl)pyridin-4-yl)-4-methylphenyl]-3-(2,2,2-trifluoroethyl)pyrrolidine-1-carboxamide (178 mg, 63%) as an light pink solid. MS ESI calculated for C₂₆H₃₄F₃N₅O₄[M+H]⁺, 538.26, found 538.10. ¹H NMR (400 MHz, DMSO-d₆) δ 8.10 (s, 1H), 7.44-7.37 (m, 1H), 7.33 (d, J=2.4 Hz, 1H), 7.09 (d, J=8.6 Hz, 1H), 6.17 (s, 1H), 5.81-5.74 (m, 2H), 4.79 (d, J=4.7 Hz, 1H), 4.52 (t, J=5.7 Hz, 1H), 3.70-3.59 (m, 6H), 3.52 (t, J=8.5 Hz, 1H), 3.43-3.33 (m, 7H), 3.18-3.07 (m, 1H), 3.01 (t, J=9.4 Hz, 1H), 2.44 (d, J=11.2 Hz, 1H), 2.40 (s, 3H), 2.15 (s, 3H), 2.12-2.04 (m, 1H), 1.72-1.58 (m, 1H). ¹⁹F NMR (376 MHz, DMSO-d₆) δ-63.36 (3F).

### Examples 88 and 89: (3S)-N-[3-(2-[[(2R)-1-hydroxypropan-2-yl]amino]-6-[(1S,5R)-3-oxabicyclo[3.1.0]hexan-1-yl]pyridin-4-yl)-4-methylphenyl]-3-(2,2,2-trifluoroethyl)pyrrolidine-1-carboxamide, (3S)-N-[3-(2-[[(2R)-1-hydroxypropan-2-yl]amino]-6-[(1R,5S)-3-oxabicyclo[3.1.0]hexan-1-yl]pyridin-4-yl)-4-methylphenyl]-3-(2,2,2-trifluoroethyl)pyrrolidine-1-carboxamide

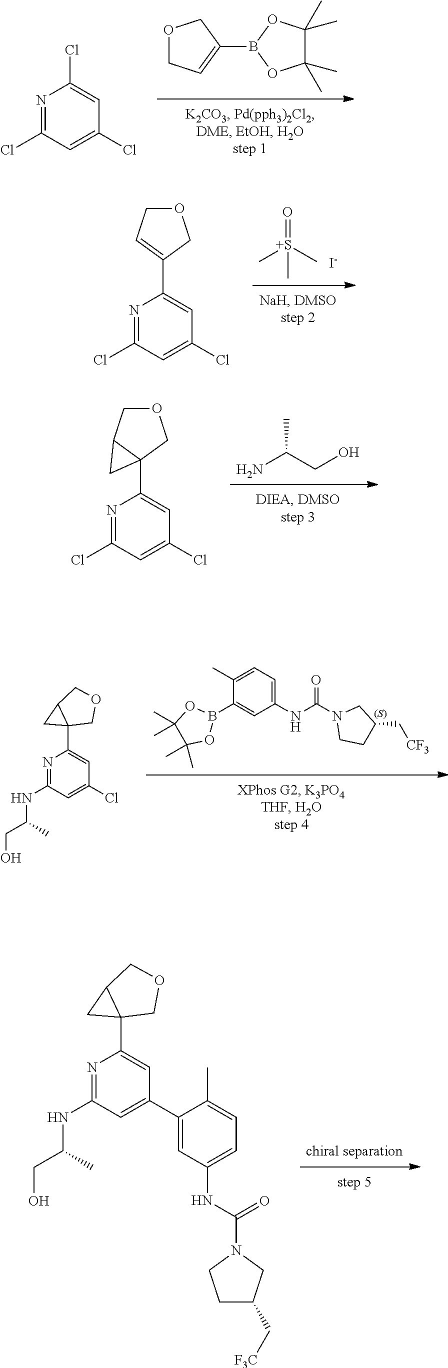

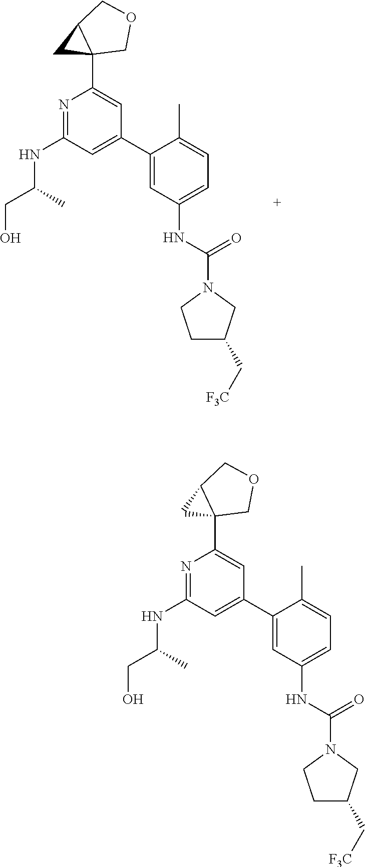 [SMILES: Cc1ccc(NC(=O)N2CC[C@@H](CC(F)(F)F)C2)cc1-c1cc(N[C@H](C)CO)nc([C@@]23COC[C@@H]2C3)c1 ; Cc1ccc(NC(=O)N2CC[C@@H](CC(F)(F)F)C2)cc1-c1cc(N[C@H](C)CO)nc([C@]23COC[C@H]2C3)c1]

Preparation 88A: 2,4-dichloro-6-(2,5-dihydrofuran-3-yl)pyridine

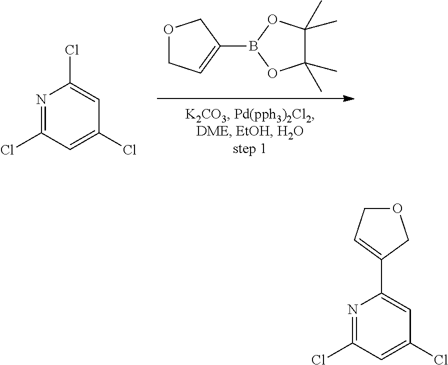

To a stirred solution of 2,4,6-trichloropyridine (5.50 g, 30.15 mmol), 2-(2,5-dihydrofuran-3-yl)-4,4,5,5-tetramethyl-1,3,2-dioxaborolane (5.91 g, 30.15 mmol) and K₂CO₃ (8.38 g, 60.60 mmol) in DME/EtOH/H₂O=2/1/2 (60 mL) was added Pd(PPh₃)₂Cl₂ (1.06 g, 1.51 mmol). The resulting mixture was stirred for 16 h at 80° C. under nitrogen atmosphere. The reaction was quenched by the addition of water (100 mL). The resulting mixture was extracted with EtOAc (3×50 mL). The combined organic layers were washed with saturated brine (3×50 mL), dried over anhydrous Na₂SO₄. After filtration, the filtrate was concentrated under reduced pressure. The residue was purified by silica gel column chromatography, eluted with PE/EtOAc (2/1) to afford 2,4-dichloro-6-(2,5-dihydrofuran-3-yl)pyridine (2.55 g, 39%) as an off-white solid. MS ESI calculated for C₉H₇Cl₂NO [M+H]⁺, 215.99, found 215.95. ¹H NMR (300 MHz, chloroform-d) δ 7.26-7.23 (m, 2H), 6.74-6.71 (m, 1H), 5.07-5.03 (m, 2H), 3.93-3.89 (m, 2H).

Preparation 88B: 2,4-dichloro-6-[3-oxabicyclo[3.1.0]hexan-1-yl]pyridine

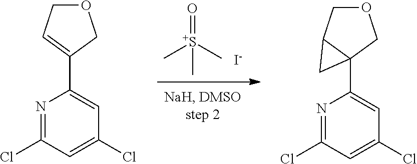

To a stirred solution of trimethylsulfoxonium iodide (10.39 g, 47.21 mmol) in DMSO (30 mL) was added NaH (1.89 g, 47.25 mmol) in portions at 0° C. The resulting mixture was stirred for 30 min at room temperature under nitrogen atmosphere. To the above mixture was added 2,4-dichloro-6-(2,5-dihydrofuran-3-yl)pyridine (2.55 g, 11.80 mmol). The resulting mixture was stirred for additional 16 h at 60° C. The reaction was quenched by the addition of water (100 mL). The resulting mixture was extracted with EtOAc (3×50 mL). The combined organic layers were washed with saturated brine (3×50 mL), dried over anhydrous Na₂SO₄. After filtration, the filtrate was concentrated under reduced pressure. The residue was purified by silica gel column chromatography, eluted with PE/EtOAc (4/1) to afford 2,4-dichloro-6-[3-oxabicyclo[3.1.0]hexan-1-yl]pyridine (2.28 g, 84%) as an off-white solid. MS ESI calculated for C₁₀H₉Cl₂NO [M+H]⁺. 230.01, found 229.95. ¹H NMR (300 MHz, chloroform-d) δ 7.18-7.17 (m, 1H), 6.97-6.96 (m, 1H), 4.14 (s, 2H), 3.95-3.86 (m, 2H), 2.21-2.16 (m, 1H), 1.46-1.42 (m, 1H), 1.28-1.19 (m, 1H).

Preparation 88C: (2R)-2-[(4-chloro-6-[3-oxabicyclo[3.1.0]hexan-1-yl]pyridin-2-yl)amino]propan-1-ol

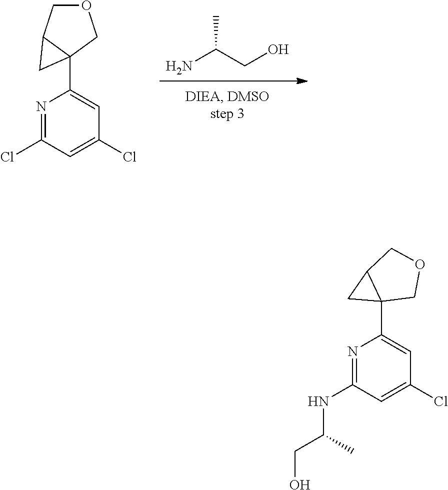

To a stirred solution of 2,4-dichloro-6-[3-oxabicyclo[3.1.0]hexan-1-yl]pyridine (2.28 g, 9.91 mmol) and (R)-(−)-2-amino-1-propanol (1.49 g, 19.82 mmol) in DMSO (25 mL) was added DIEA (2.56 g, 19.82 mmol) dropwise at room temperature. The resulting mixture was stirred for 16 h at 130° C. The reaction was quenched by the addition of water (100 mL). The resulting mixture was extracted with EtOAc (3×50 mL). The combined organic layers were washed with saturated brine (3×50 mL), dried over anhydrous Na₂SO₄. After filtration, the filtrate was concentrated under reduced pressure. The residue was purified by reverse flash chromatography with the following conditions: column, C18 silica gel; mobile phase, CH₃CN in water (0.1% TFA), 5% to 95% gradient in 40 min; detector, UV 254 nm; to afford (2R)-2-[(4-chloro-6-[3-oxabicyclo[3.1.0]hexan-1-yl]pyridin-2-yl)amino]propan-1-ol (0.38 g, 14%) as an off-white solid. MS ESI calculated for C₁₃H₁₇ClN₂O₂[M+H]⁺. 269.10, found 269.00. ¹H NMR (300 MHz, chloroform-d) δ 6.38-6.36 (m, 1H), 6.31-6.30 (m, 1H), 4.72 (s, 1H), 4.09 (s, 2H), 4.04-3.84 (m, 3H), 3.78-3.73 (m, 1H), 3.61-3.54 (m, 1H), 2.14-2.07 (m, 1H), 1.38-1.31 (m, 1H), 1.28-1.24 (m, 4H), 1.11-1.07 (m, 1H).

Preparation 88D: (3S)-N-[3-(2-[[(2R)-1-hydroxypropan-2-yl]amino]-6-[3-oxabicyclo[3.1.0]hexan-1-yl]pyridin-4-yl)-4-methylphenyl]-3-(2,2,2-trifluoroethyl)pyrrolidine-1-carboxamide

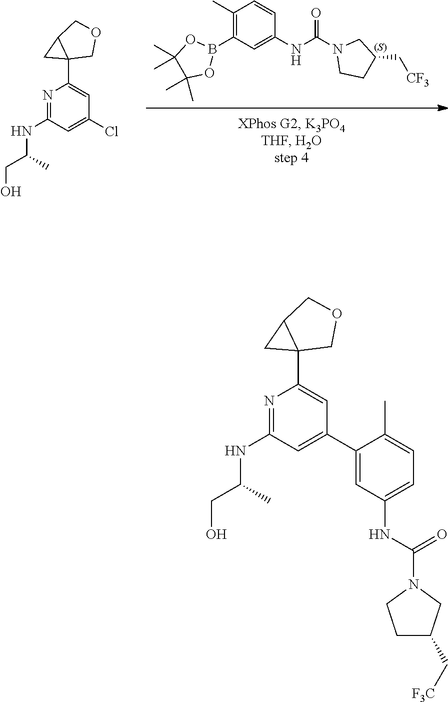

To a stirred solution of (2R)-2-[(4-chloro-6-[3-oxabicyclo[3.1.0]hexan-1-yl]pyridin-2-yl)amino]propan-1-ol (380 mg, 1.414 mmol), (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (583 mg, 1.414 mmol) and K₃PO₄ (600 mg, 2.828 mmol) in THF/H₂O=10/1 (4 mL) was added XPhos Pd G2 (111 mg, 0.141 mmol) at room temperature under nitrogen atmosphere. The resulting mixture was stirred for 16 h at 80° C. under nitrogen atmosphere. The reaction was quenched by the addition of water (100 mL). The resulting mixture was extracted with EtOAc (3×50 mL). The combined organic layers were washed with saturated brine (3×50 mL), dried over anhydrous Na₂SO₄. After filtration, the filtrate was concentrated under reduced pressure. The residue was purified by silica gel column chromatography, eluted with (EA/PE/EtOH=8/3/1) to afford (3S)-N-[3-(2-[[(2R)-1-hydroxypropan-2-yl]amino]-6-[3-oxabicyclo[3.1.0]hexan-1-yl]pyridin-4-yl)-4-methylphenyl]-3-(2,2,2-trifluoroethyl)pyrrolidine-1-carboxamide (420 mg, 57%) as a light yellow solid. MS ESI calculated for C₂₇H₃₃F₃N₄O₃[M+H]⁺. 519.25, found 519.25. ¹H NMR (300 MHz, chloroform-d) δ 7.31-7.29 (m, 1H), 7.19-7.16 (m, 1H), 6.34-6.32 (m, 1H), 6.24-6.22 (m, 2H), 4.59 (s, 1H), 4.18-4.04 (m, 3H), 3.93-3.75 (m, 4H), 3.67-3.55 (m, 2H), 3.49-3.40 (m, 1H), 3.15-3.08 (m, 1H), 2.62-2.49 (m, 1H), 2.34-2.11 (m, 7H), 1.81-1.68 (m, 2H), 1.40-1.30 (m, 1H), 1.27-1.24 (m, 3H), 1.09-1.05 (m, 1H). ¹⁹F NMR (282 MHz, chloroform-d) δ-64.94 (3F).

### Examples 88 and 89: (3S)-N-[3-(2-[[(2R)-1-hydroxypropan-2-yl]amino]-6-[(1S,5R)-3-oxabicyclo[3.1.0]hexan-1-yl]pyridin-4-yl)-4-methylphenyl]-3-(2,2,2-trifluoroethyl)pyrrolidine-1-carboxamide, (3S)-N-[3-(2-[[(2R)-1-hydroxypropan-2-yl]amino]-6-[(1R,5S)-3-oxabicyclo[3.1.0]hexan-1-yl]pyridin-4-yl)-4-methylphenyl]-3-(2,2,2-trifluoroethyl)pyrrolidine-1-carboxamide

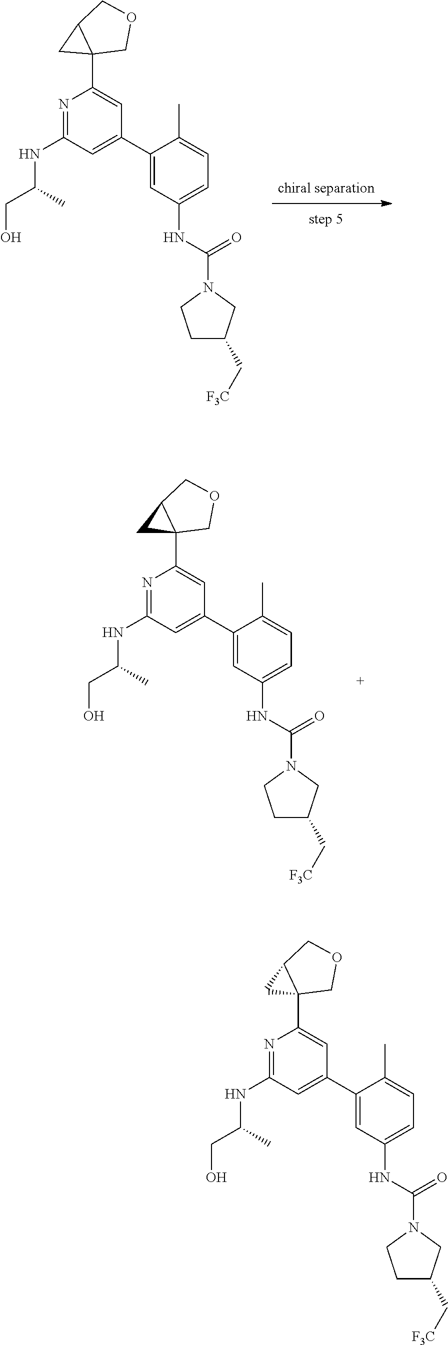 [SMILES: Cc1ccc(NC(=O)N2CC[C@@H](CC(F)(F)F)C2)cc1-c1cc(N[C@H](C)CO)nc(C23COCC2C3)c1 ; Cc1ccc(NC(=O)N2CC[C@@H](CC(F)(F)F)C2)cc1-c1cc(N[C@H](C)CO)nc([C@@]23COC[C@@H]2C3)c1 ; Cc1ccc(NC(=O)N2CC[C@@H](CC(F)(F)F)C2)cc1-c1cc(N[C@H](C)CO)nc([C@]23COC[C@H]2C3)c1]

The crude product ((3S)-N-[3-(2-[[(2R)-1-hydroxypropan-2-yl]amino]-6-[3-oxabicyclo[3.1.0]hexan-1-yl]pyridin-4-yl)-4-methylphenyl]-3-(2,2,2-trifluoroethyl)pyrrolidine-1-carboxamide (420 mg, 0.810 mmol) was purified by Chiral-Prep-HPLC with the following conditions (Column: CHIRAL ART Cellulose-SB, 30*250 mm, 5 um; Mobile Phase A:CO₂, Mobile Phase B:IPA (0.5% 2 M NH₃-MeOH); Flow rate:80 mL/min; Gradient:40% B; 220 nm). The first peak afforded 169 mg (40%) as an off-white solid. MS ESI calculated for C₂₇H₃₃F₃N₄O₃ [M+H]⁺. 519.25, found 519.10. ¹H NMR (400 MHz, chloroform-d) δ 7.28 (m, 1H), 7.17-7.14 (m, 1H), 6.31-6.30 (m, 1H), 6.21-6.19 (m, 2H), 4.99-4.56 (m, 2H), 4.09-4.02 (m, 3H), 3.91-3.73 (m, 4H), 3.62-3.53 (m, 2H), 3.44-3.40 (m, 1H), 3.13-3.07 (m, 1H), 2.60-2.49 (m, 1H), 2.32-2.09 (m, 7H), 1.80-1.67 (m, 2H), 1.39-1.35 (m, 1H), 1.24-1.22 (m, 3H), 1.08-1.05 (m, 1H). ¹⁹F NMR (376 MHz, chloroform-d) δ-64.95 (3F).

The second peak afforded 119 mg (28%) as an off-white solid. MS ESI calculated for C₂₇H₃₃F₃N₄O₃[M+H]⁺. 519.25, found 519.10. ¹H NMR (400 MHz, chloroform-d) δ 7.31-7.30 (m, 1H), 7.20-7.17 (m, 1H), 6.35 (m, 1H), 6.25-6.19 (m, 2H), 4.63 (m, 2H), 4.11-4.04 (m, 3H), 3.94-3.75 (m, 4H), 3.64-3.56 (m, 2H), 3.47-3.43 (m, 1H), 3.19-3.10 (m, 1H), 2.58-2.55 (m, 1H), 2.32-2.14 (m, 7H), 1.83-1.72 (m, 2H), 1.35-1.31 (m, 1H), 1.28-1.24 (m, 3H), 1.09-1.06 (m, 1H). ¹⁹F NMR (376 MHz, chloroform-d) δ-64.95 (3F).

### Example 90 and 91: (S)-N-(3-(6-(((R)-2-hydroxypropyl)amino)-2-morpholinopyrimidin-4-yl)-4-methylphenyl)-3-(2,2,2-trifluoroethyl)pyrrolidine-1-carboxamide & (R)-N-(3-(6-(((R)-2-hydroxypropyl)amino)-2-morpholinopyrimidin-4-yl)-4-methylphenyl)-3-(2,2,2-trifluoroethyl)pyrrolidine-1-carboxamide

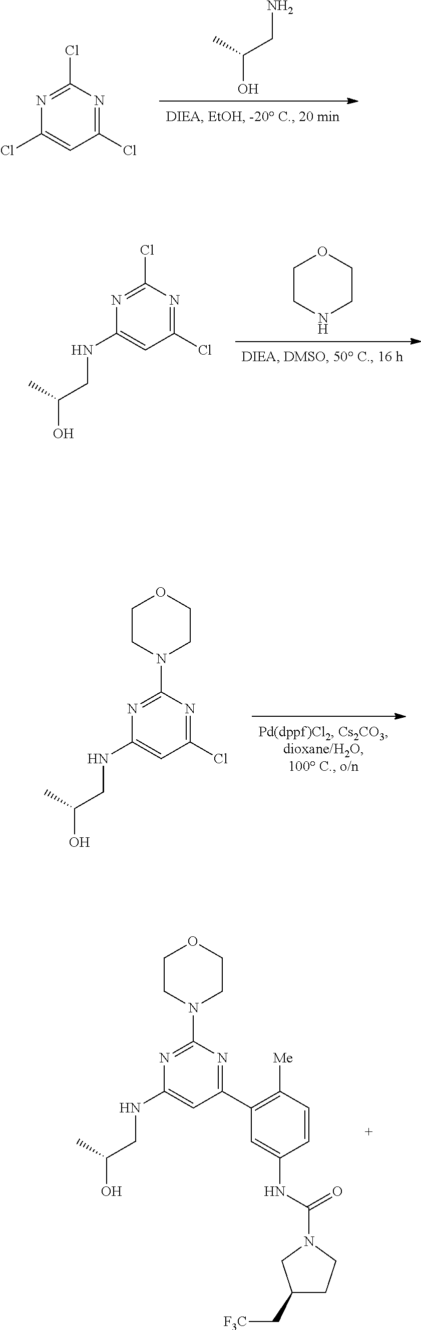

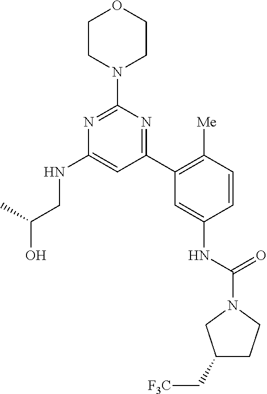 [SMILES: Cc1ccc(NC(=O)N2CC[C@H](CC(F)(F)F)C2)cc1-c1cc(NC[C@@H](C)O)nc(N2CCOCC2)n1]

Step 1: To a stirred solution of 2,4,6-trichloropyrimidine (4.00 g, 21.85 mmol) in EtOH (70 mL) was added (R)-1-aminopropan-2-ol (1.63 g, 21.85 mmol) and DIEA (2.82 g, 21.85 mmol) at −20° C. The reaction mixture was stirred at −20° C. for 20 min. The resulting mixture was diluted with water (150 mL) and extracted with EA (300 mL×3). The combined organic layers was washed with brine (100 mL×4), dried over anhydrous Na₂SO₄ and filtered. The filtrate was concentrated under reduced pressure. The residue was purified by FCC (PE:EA=5:1) to afford (R)-1-((2,6-dichloropyrimidin-4-yl)amino)propan-2-ol (2.30 g, 48%) as an off-white solid. MS Calcd.: 221, MS Found: 222 ([M+H]⁺).
Step 2: To a stirred solution of (R)-1-((2,6-dichloropyrimidin-4-yl)amino)propan-2-ol (1.00 g, 4.52 mmol) in DMSO (5 mL) were added morpholine (396 mg, 4.52 mmol) and DIEA (875 mg, 6.78 mmol) at rt. The reaction mixture was stirred at 50° C. for 16 h. The resulting mixture was cooled to rt, diluted with water (10 mL) and extracted with EA (10 mL×3). The combined organic layers was washed with brine (5 mL×4), dried over anhydrous Na₂SO₄ and filtered. The filtrate was concentrated under reduced pressure. The residue was purified by FCC (PE:EA=1:2) to afford (R)-1-((6-chloro-2-morpholinopyrimidin-4-yl)amino)propan-2-ol (1.07 g, 86%) as a white solid. MS Calcd.: 272, MS Found: 273 ([M+H]⁺).
Step 3: To a solution of (R)-1-((6-chloro-2-morpholinopyrimidin-4-yl)amino)propan-2-ol (600 mg, 2.20 mmol), N-(4-methyl-3-(4,4,5,5-tetramethyl-1,3,2-dioxaborolan-2-yl)phenyl)-3-(2,2,2-trifluoroethyl)pyrrolidine-1-carboxamide (996 mg, 2.42 mmol) and Cs₂CO₃ (1.79 g, 2.49 mmol) in dioxane (40 mL) and water (4 mL) was added Pd(dppf)Cl₂ (80 mg, 0.11 mmol) at room temperature under nitrogen atmosphere. The reaction mixture was stirred at 100° C. overnight.

The mixture was cooled down to room temperature, filtered and concentrated. The residue was purified by chiral prep-SFC (Column: Chiralpak IG 5 μm*20*250 mm; Mobile Phase: CO₂: EtOH=70:30 at 50 g/min; Temp: 40° C.; Wavelength: 214 nm, Back pressure: 100 bar) to afford 183.8 mg (16%), RT=7.13 min, ee>98%), ¹H NMR (400 MHz, DMSO-d₆): δ 8.13 (s, 1H), 7.49 (d, J=2.4 Hz, 1H), 7.42 (dd, J=2.0, 8.4 Hz, 1H), 7.07 (d, J=8.4 Hz, 2H), 5.90 (s, 1H), 4.71 (br s, 1H), 3.80-3.79 (m, 1H), 3.69-3.63 (m, 9H), 3.55-3.50 (m, 1H), 3.34-3.20 (m, 3H), 3.02 (t, J=9.2 Hz, 1H), 2.49-2.38 (m, 3H), 2.25 (s, 3H), 2.09-2.07 (m, 1H), 1.68-1.63 (m, 1H), 1.08 (d, J=6.4 Hz, 3H) and 196.7 mg, (17%), RT=6.31 min, ee>98%), 1H NMR (400 MHz, DMSO-d₆): δ 8.13 (s, 1H), 7.49 (d, J=2.4 Hz, 1H), 7.42 (dd, J=2.4, 8.4 Hz, 1H), 7.08-7.03 (m, 2H), 5.90 (s, 1H), 4.71 (d, J=4.0 Hz, 1H), 3.81-3.78 (m, 1H), 3.69-3.63 (m, 9H), 3.55-3.50 (m, 1H), 3.34-3.20 (m, 3H), 3.02 (t, J=9.2 Hz, 1H), 2.49-2.38 (m, 3H), 2.25 (s, 3H), 2.09-2.07 (m, 1H), 1.68-1.63 (m, 1H), 1.08 (d, J=6.4 Hz, 3H). MS Calcd.: 522, MS Found: 523 ([M+H]⁺).

### Example 92: (S)-N-(3-(2-(((R)-2-hydroxypropyl)amino)-6-morpholinopyrimidin-4-yl)-4-methylphenyl)-3-(2,2,2-trifluoroethyl)pyrrolidine-1-carboxamide

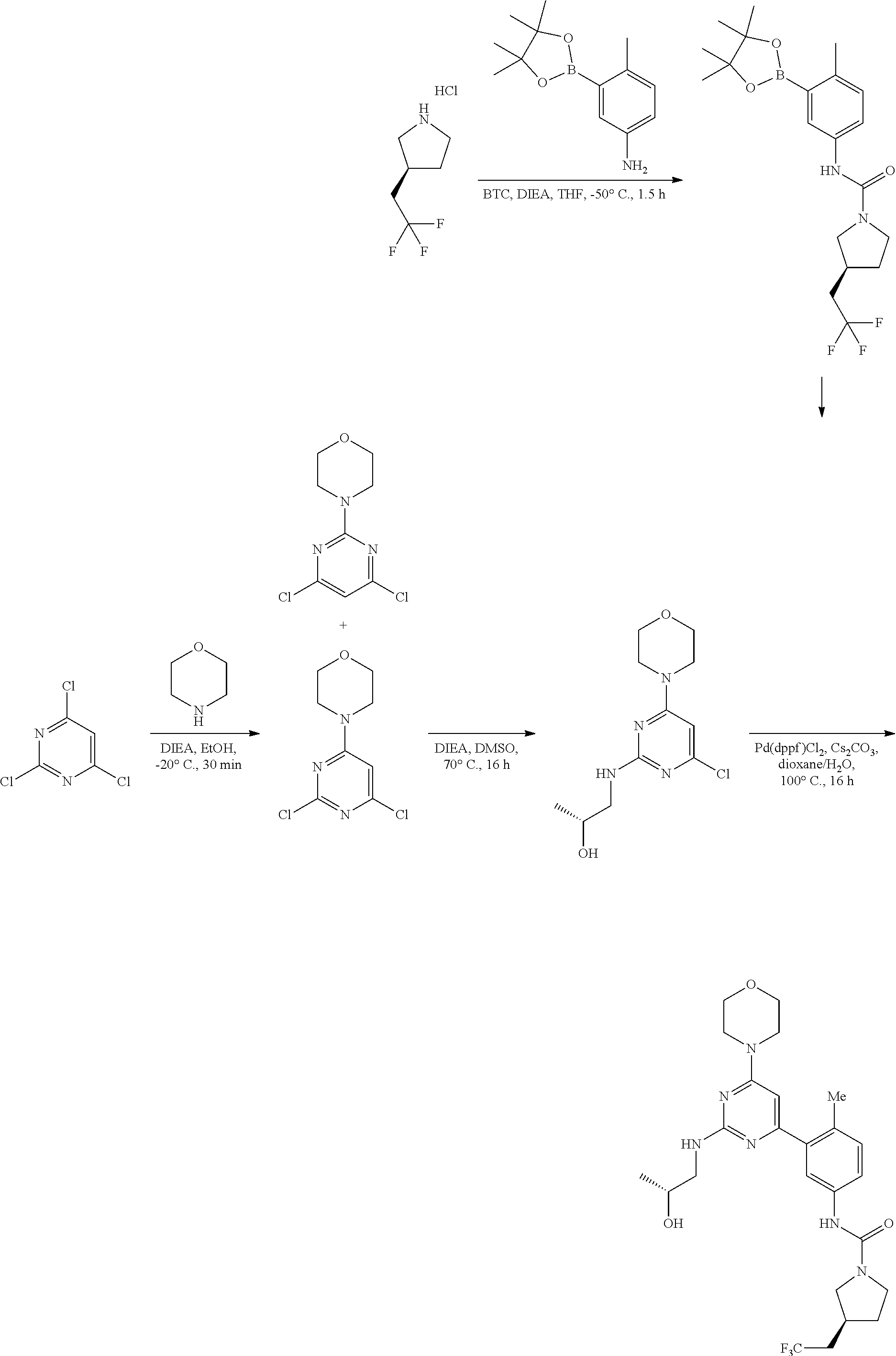

Step 1: To a solution of 4-methyl-3-(4,4,5,5-tetramethyl-1,3,2-dioxaborolan-2-yl)aniline (3.80 g, 16.31 mmol) and DIEA (10.5 g, 81.50 mmol) in THF (100 mL) was added BTC (1.9 g, 6.40 mmol) at −50° C. under nitrogen atmosphere. The mixture was stirred at −50° C. for 30 min. Then (S)-3-(2,2,2-trifluoroethyl)pyrrolidine hydrochloride (3.1 g, 16.40 mmol) was added to the mixture which was stirred at −50° C. for another 1 h. The residue was concentrated and purified by FCC (PE:EA=1:1) to afford (S)-N-(4-methyl-3-(4,4,5,5-tetramethyl-1,3,2-dioxaborolan-2-yl)phenyl)-3-(2,2,2-trifluoroethyl)pyrrolidine-1-carboxamide (3.85 g, 57%) as a white solid. MS Calcd.: 412, MS Found: 413 ([M+H]⁺).
Step 2: To a stirred solution of 2,4,6-trichloropyrimidine (5.00 g, 27.47 mmol) in EtOH (70 mL) was added morpholine (2.39 g, 27.47 mmol) and DIEA (3.51 g, 27.47 mmol) at −20° C. The reaction mixture was stirred at −20° C. for 0.5 h. The resulting mixture was diluted with water (150 mL) and extracted with EA (300 mL×3). The combined organic layers was washed with brine (100 mL×4), dried over anhydrous Na2SO4 and filtered. The filtrate was concentrated under reduced pressure. The residue was purified by FCC (PE:EA=10:1) to afford 4-(4,6-dichloropyrimidin-2-yl)morpholine (1.13 g, 17%) and 4-(2,6-dichloropyrimidin-4-yl)morpholine (3.58 g, 54%) as an off-white solid. MS Calcd.: 233, MS Found: 234 ([M+H]⁺).
Step 3: To a stirred solution of 4-(2,6-dichloropyrimidin-2-yl)morpholine (600 mg, 2.36 mmol) in DMSO (5 mL) were added (R)-1-aminopropan-2-ol (250 mg, 3.33 mmol) and DIEA (496 mg, 3.84 mmol) at rt. The reaction mixture was stirred at 70° C. for 16 h. The resulting mixture was cooled to rt, diluted with water (10 mL) and extracted with EA (10 mL×3). The combined organic layers was washed with brine (5 mL×4), dried over anhydrous Na₂SO₄ and filtered. The filtrate was concentrated under reduced pressure. The residue was purified by FCC (PE:EA=1:1) to afford (R)-1-((4-chloro-6-morpholinopyrimidin-2-yl)amino)propan-2-ol (400 mg, 57%) as a white solid. MS Calcd.: 272, MS Found: 273 ([M+H]⁺).
Step 4: To a solution of (R)-1-((4-chloro-6-morpholinopyrimidin-2-yl)amino)propan-2-ol (33 mg, 0.12 mmol), (S)-N-(4-methyl-3-(4,4,5,5-tetramethyl-1,3,2-dioxaborolan-2-yl)phenyl)-3-(2,2,2-trifluoroethyl)pyrrolidine-1-carboxamide (50 mg, 0.12 mmol) and Cs₂CO₃ (98 mg, 0.30 mmol) in dioxane (10 mL) and water (2 mL) was added Pd(dppf)Cl₂ (9 mg, 0.012 mmol) at room temperature under nitrogen atmosphere. The reaction mixture was stirred at 100° C. for 16 h. The mixture was cooled down to room temperature, filtered and concentrated. The residue was purified by prep-HPLC to afford (S)-N-(3-(2-(((R)-2-hydroxypropyl)amino)-6-morpholinopyrimidin-4-yl)-4-methylphenyl)-3-(2,2,2-trifluoroethyl)pyrrolidine-1-carboxamide (21 mg, 33%) as a white solid. ¹H NMR (400 MHz, DMSO-d₆): δ 8.12 (s, 1H), 7.45-7.43 (m, 2H), 7.07 (dd, J=3.6, 4.8 Hz, 1H), 6.40 (s, 1H), 6.02 (s, 1H), 4.76-4.71 (m, 1H), 3.76-3.50 (m, 12H), 3.29-3.14 (m, 2H), 3.02 (t, J=9.2 Hz, 1H), 2.46-2.33 (m, 3H), 2.24 (s, 3H), 2.11-2.07 (m, 1H), 1.70-1.63 (m, 1H), 1.04 (d, J=6.0 Hz, 3H).MS Calcd.: 522, MS Found: 523 ([M+H]⁺).

### Example 93: (S)-N-(3-(6-(((S)-2-hydroxypropyl)amino)-2-morpholinopyrimidin-4-yl)-4-methylphenyl)-3-(2,2,2-trifluoroethyl)pyrrolidine-1-carboxamide

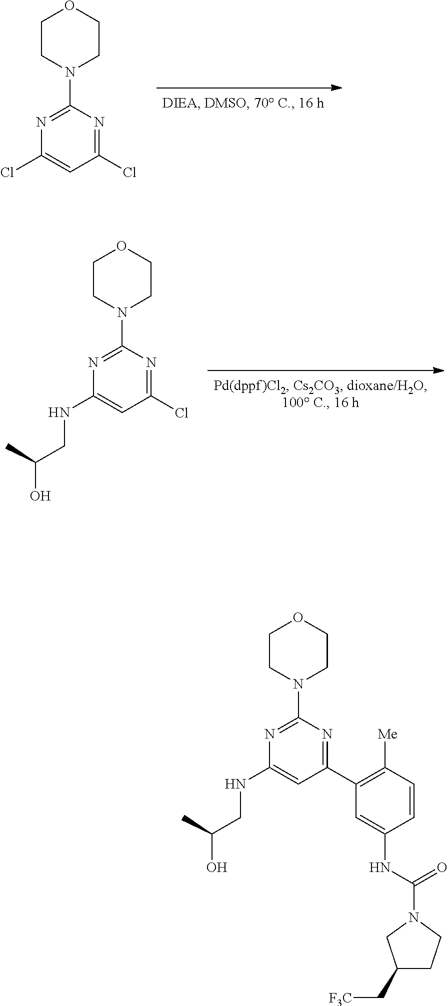 [SMILES: Clc1cc(Cl)nc(N2CCOCC2)n1 ; C[C@H](O)CNc1cc(Cl)nc(N2CCOCC2)n1 ; Cc1ccc(NC(=O)N2CC[C@@H](CC(F)(F)F)C2)cc1-c1cc(NC[C@H](C)O)nc(N2CCOCC2)n1]

Step 1: To a stirred solution of 4-(4,6-dichloropyrimidin-2-yl)morpholine (300 mg, 1.28 mmol) in DMSO (5 mL) was added (S)-1-aminopropan-2-ol (96 mg, 1.28 mmol) and DIEA (248 mg, 1.92 mmol) at rt. The reaction mixture was stirred at 70° C. for 16 h. The resulting mixture was cooled to rt, diluted with water (10 mL) and extracted with EA (10 mL×3). The combined organic layers was washed with brine (5 mL×4), dried over anhydrous Na2SO4 and filtered. The filtrate was concentrated under reduced pressure. The residue was purified by FCC (PE:EA=1:1) to afford (S)-1-((6-chloro-2-morpholinopyrimidin-4-yl)amino)propan-2-ol (200 mg, 57%) as a white solid. MS Calcd.: 272, MS Found: 273 ([M+H]⁺).
Step 2: To a solution of (S)-1-((6-chloro-2-morpholinopyrimidin-4-yl)amino)propan-2-ol (33 mg, 0.12 mmol), (S)-N-(4-methyl-3-(4,4,5,5-tetramethyl-1,3,2-dioxaborolan-2-yl)phenyl)-3-(2,2,2-trifluoroethyl)pyrrolidine-1-carboxamide (50 mg, 0.12 mmol) and Cs₂CO₃ (98 mg, 0.30 mmol) in dioxane (10 mL) and water (2 mL) was added Pd(dppf)Cl₂ (4 mg, 0.006 mmol) at room temperature under nitrogen atmosphere. The reaction mixture was stirred at 100° C. for 16 h. The mixture was cooled down to room temperature, filtered and concentrated. The residue was purified by prep-HPLC to afford (S)-N-(3-(6-(((S)-2-hydroxypropyl)amino)-2-morpholinopyrimidin-4-yl)-4-methylphenyl)-3-(2,2,2-trifluoroethyl)pyrrolidine-1-carboxamide (15.7 mg, 24%) as a white solid. 1H NMR (400 MHz, DMSO-d₆): δ 8.13 (d, J=1.6 Hz, 1H), 7.49 (d, J=2.4 Hz, 1H), 7.42 (dd, J=2.0, 8.0 Hz, 1H), 7.08-7.05 (m, 2H), 5.90 (s, 1H), 4.70 (d, J=4.4 Hz, 1H), 3.81-3.78 (m, 1H), 3.69-3.63 (m, 9H), 3.55-3.50 (m, 1H), 3.29-3.24 (m, 3H), 3.02 (t, J=9.2 Hz, 1H), 2.49-2.38 (m, 3H), 2.25 (s, 3H), 2.10-2.07 (m, 1H), 1.68-1.63 (m, 1H), 1.07 (d, J=6.0 Hz, 3H). MS Calcd.: 522, MS Found: 523 ([M+H]⁺).

### Example 94: (S)-N-(3-(6-((2-hydroxyethyl)amino)-2-morpholinopyrimidin-4-yl)-4-methylphenyl)-3-(2,2,2-trifluoroethyl)pyrrolidine-1-carboxamide

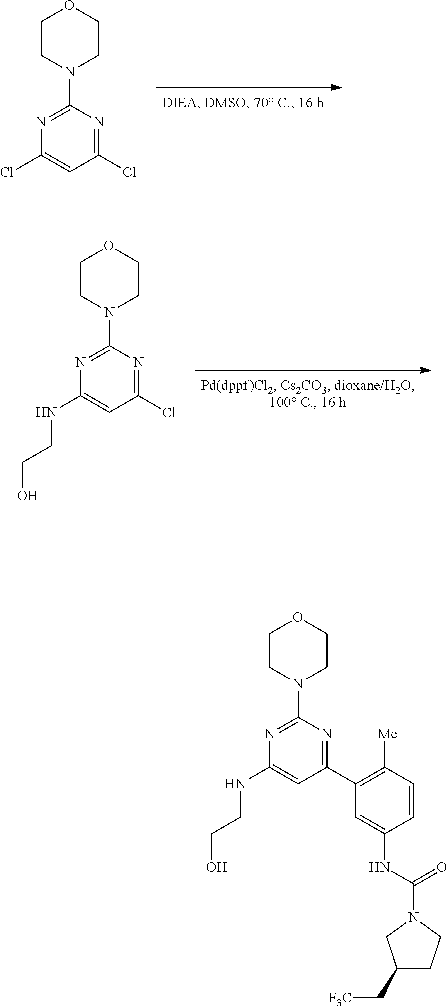 [SMILES: Clc1cc(Cl)nc(N2CCOCC2)n1 ; OCCNc1cc(Cl)nc(N2CCOCC2)n1 ; Cc1ccc(NC(=O)N2CC[C@@H](CC(F)(F)F)C2)cc1-c1cc(NCCO)nc(N2CCOCC2)n1]

Step 1: To a stirred solution of 4-(4,6-dichloropyrimidin-2-yl)morpholine (300 mg, 1.28 mmol) in DMSO (5 mL) were added 2-aminoethanol (102 mg, 1.17 mmol) and DIEA (248 mg, 1.92 mmol) at rt. The reaction mixture was stirred at 70° C. for 16 h. The resulting mixture was cooled to rt, diluted with water (10 mL) and extracted with EA (10 mL×3). The combined organic layers was washed with brine (5 mL×4), dried over anhydrous Na₂SO₄ and filtered. The filtrate was concentrated under reduced pressure. The residue was purified by FCC (PE:EA=1:1) to afford 2-((6-chloro-2-morpholinopyrimidin-4-yl)amino)ethanol (325 mg, 98%) as a white solid. MS Calcd.: 258, MS Found: 259 ([M+H]⁺).
Step 2: To a solution of 2-((6-chloro-2-morpholinopyrimidin-4-yl)amino)ethanol (31 mg, 0.12 mmol), (S)-N-(4-methyl-3-(4,4,5,5-tetramethyl-1,3,2-dioxaborolan-2-yl)phenyl)-3-(2,2,2-trifluoroethyl)pyrrolidine-1-carboxamide (50 mg, 0.12 mmol) and Cs₂CO₃ (98 mg, 0.30 mmol) in dioxane (10 mL) and water (2 mL) was added Pd(dppf)Cl₂ (9 mg, 0.012 mmol) at room temperature under nitrogen atmosphere. The reaction mixture was stirred at 100° C. for 16 h. The mixture was cooled down to room temperature, filtered and concentrated. The residue was purified by prep-HPLC to afford (S)-N-(3-(6-((2-hydroxyethyl)amino)-2-morpholinopyrimidin-4-yl)-4-methylphenyl)-3-(2,2,2-trifluoroethyl)pyrrolidine-1-carboxamide (17 mg, 27.4%) as a white solid. ¹H NMR (400 MHz, DMSO-d₆): δ 8.13 (s, 1H), 7.49 (d, J=2.4 Hz, 1H), 7.42 (dd, J=2.0, 8.8 Hz, 1H), 7.07 (d, J=8.4 Hz, 1H), 5.88 (s, 1H), 4.69 (t, J=5.2 Hz, 1H), 3.69-3.63 (m, 9H), 3.55-3.51 (m, 3H), 3.36-3.27 (m, 3H), 3.02 (t, J=9.6 Hz, 1H), 2.49-2.38 (m, 3H), 2.25 (s, 3H), 2.09-2.07 (m, 1H), 1.68-1.63 (m, 1H). MS Calcd.: 508, MS Found: 509 ([M+H]⁺).

### Example 95: (S)-N-(3-(6-(((S)-1-hydroxypropan-2-yl)amino)-2-morpholinopyrimidin-4-yl)-4-methylphenyl)-3-(2,2,2-trifluoroethyl)pyrrolidine-1-carboxamide

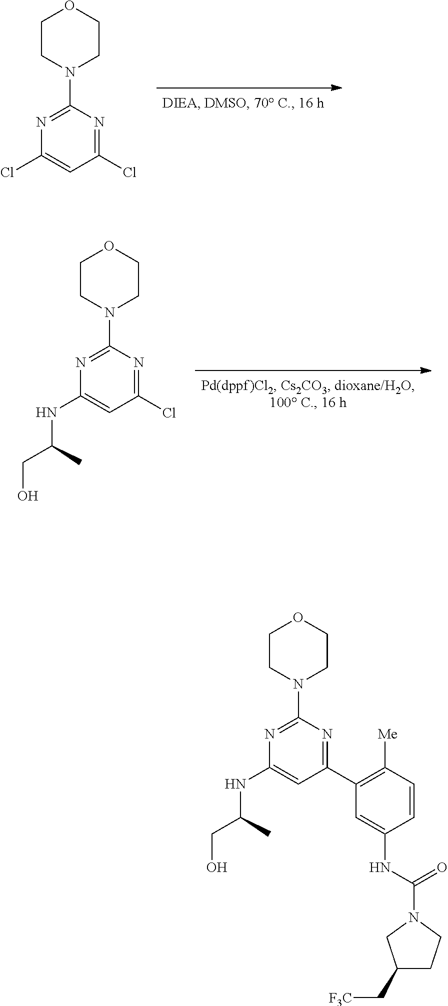 [SMILES: Clc1cc(Cl)nc(N2CCOCC2)n1 ; C[C@@H](CO)Nc1cc(Cl)nc(N2CCOCC2)n1 ; Cc1ccc(NC(=O)N2CC[C@@H](CC(F)(F)F)C2)cc1-c1cc(N[C@@H](C)CO)nc(N2CCOCC2)n1]

Step 1: To a stirred solution of 4-(4,6-dichloropyrimidin-2-yl)morpholine (300 mg, 1.28 mmol) in DMSO (5 mL) were added (S)-2-aminopropan-1-ol (125 mg, 1.66 mmol) and DIEA (248 mg, 1.92 mmol) at rt. The reaction mixture was stirred at 70° C. for 16 h. The resulting mixture was diluted with water (10 mL) and extracted with EA (10 mL×3). The combined organic layers was washed with brine (5 mL×4), dried over anhydrous Na₂SO₄ and filtered. The filtrate was concentrated under reduced pressure. The residue was purified by FCC (PE:EA=1:1) to afford (S)-2-((6-chloro-2-morpholinopyrimidin-4-yl)amino)propan-1-ol (270 mg, 77%) as a white solid. MS Calcd.: 272, MS Found: 273 ([M+H]⁺).
Step 2: To a solution of (S)-2-((6-chloro-2-morpholinopyrimidin-4-yl)amino)propan-1-ol (33 mg, 0.12 mmol), (S)-N-(4-methyl-3-(4,4,5,5-tetramethyl-1,3,2-dioxaborolan-2-yl)phenyl)-3-(2,2,2-trifluoroethyl)pyrrolidine-1-carboxamide (50 mg, 0.12 mmol) and Cs₂CO₃ (98 mg, 0.30 mmol) in dioxane (10 mL) and water (2 mL) was added Pd(dppf)Cl₂ (4 mg, 0.006 mmol) at room temperature under nitrogen atmosphere. The reaction mixture was stirred at 100° C. for 16 h. The mixture was cooled down to room temperature, filtered and concentrated. The residue was purified by prep-HPLC to afford (S)-N-(3-(6-(((S)-1-hydroxypropan-2-yl)amino)-2-morpholinopyrimidin-4-yl)-4-methylphenyl)-3-(2,2,2-trifluoroethyl)pyrrolidine-1-carboxamide (11.4 mg, 18%) as a white solid. ¹H NMR (400 MHz, DMSO-d₆): δ 8.12 (s, 1H), 7.50 (s, 1H), 7.41 (d, J=8.4 Hz, 1H), 7.08-6.81 (m, 2H), 5.85 (s, 1H), 4.70-4.69 (m, 1H), 4.03 (br s, 1H), 3.69-3.63 (m, 9H), 3.55-3.45 (m, 1H), 3.38-3.27 (m, 3H), 3.02 (t, J=9.2 Hz, 1H), 2.49-2.38 (m, 3H), 2.26 (s, 3H), 2.10-2.07 (m, 1H), 1.68-1.63 (m, 1H), 1.13 (d, J=6.8 Hz, 3H). MS Calcd.: 522, MS Found: 523 ([M+H]⁺).

### Example 96: (S)-N-(3-(6-((2-hydroxy-2-methylpropyl)amino)-2-morpholinopyrimidin-4-yl)-4-methylphenyl)-3-(2,2,2-trifluoroethyl)pyrrolidine-1-carboxamide

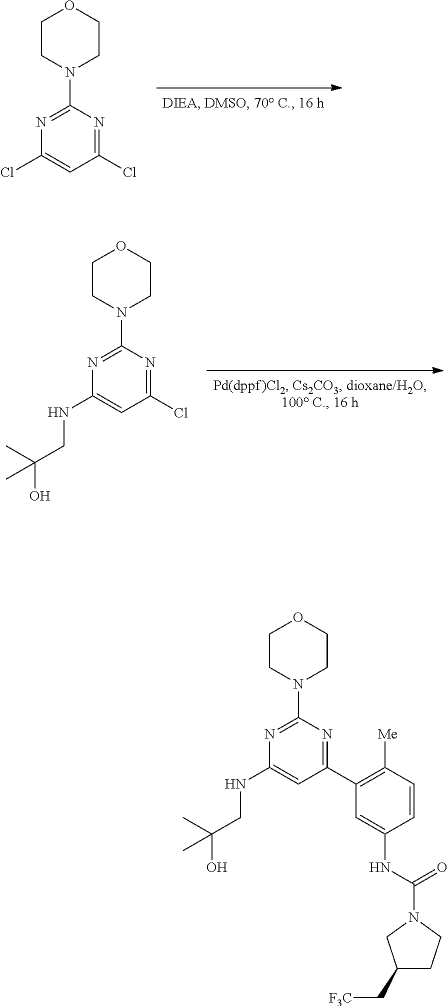 [SMILES: Clc1cc(Cl)nc(N2CCOCC2)n1 ; CC(C)(O)CNc1cc(Cl)nc(N2CCOCC2)n1 ; Cc1ccc(NC(=O)N2CC[C@@H](CC(F)(F)F)C2)cc1-c1cc(NCC(C)(C)O)nc(N2CCOCC2)n1]

Step 1: To a stirred solution of 4-(4,6-dichloropyrimidin-2-yl)morpholine (200 mg, 0.85 mmol) in DMSO (5 mL) were added 1-amino-2-methylpropan-2-ol (91 mg, 1.03 mmol) and DIEA (165 mg, 1.28 mmol) at rt. The reaction mixture was stirred at 70° C. for 16 h. The resulting mixture was diluted with water (10 mL) and extracted with EA (10 mL×3). The combined organic layers was washed with brine (5 mL×4), dried over anhydrous Na₂SO₄ and filtered. The filtrate was concentrated under reduced pressure. The residue was purified by FCC (PE:EA=2:1) to afford 1-((6-chloro-2-morpholinopyrimidin-4-yl)amino)-2-methylpropan-2-ol (150 mg, 61%) as a white solid. MS Calcd.: 286, MS Found: 287 ([M+H]⁺).
Step 2: To a solution of 1-((6-chloro-2-morpholinopyrimidin-4-yl)amino)-2-methylpropan-2-ol (35 mg, 0.12 mmol), (S)-N-(4-methyl-3-(4,4,5,5-tetramethyl-1,3,2-dioxaborolan-2-yl)phenyl)-3-(2,2,2-trifluoroethyl)pyrrolidine-1-carboxamide (50 mg, 0.12 mmol) and Cs₂CO₃ (98 mg, 0.30 mmol) in dioxane (10 mL) and water (2 mL) was added Pd(dppf)Cl2 (4 mg, 0.006 mmol) at room temperature under nitrogen atmosphere. The reaction mixture was stirred at 100° C. for 16 h. The mixture was cooled down to room temperature, filtered and concentrated. The residue was purified by prep-HPLC to afford (S)-N-(3-(6-((2-hydroxy-2-methylpropyl)amino)-2-morpholinopyrimidin-4-yl)-4-methylphenyl)-3-(2,2,2-trifluoroethyl)pyrrolidine-1-carboxamide (7.9 mg, 12%) as a white solid. 1H NMR (400 MHz, DMSO-d₆): δ 8.13 (s, 1H), 7.50 (d, J=2.0 Hz, 1H), 7.43 (dd, J=2.4, 8.4 Hz, 1H), 7.08-6.64 (m, 2H), 5.98 (s, 1H), 4.53 (s, 1H), 3.69-3.50 (m, 10H), 3.37-3.23 (m, 3H), 3.02 (t, J=9.2 Hz, 1H), 2.46-2.33 (m, 3H), 2.26 (s, 3H), 2.11-2.07 (m, 1H), 1.70-1.60 (m, 1H), 1.07 (s, 6H). MS Calcd.: 536, MS Found: 537 ([M+H]⁺).

### Example 97: (S)-N-(3-(2-((2-hydroxyethyl)amino)-6-morpholinopyrimidin-4-yl)-4-methylphenyl)-3-(2,2,2-trifluoroethyl)pyrrolidine-1-carboxamide formate

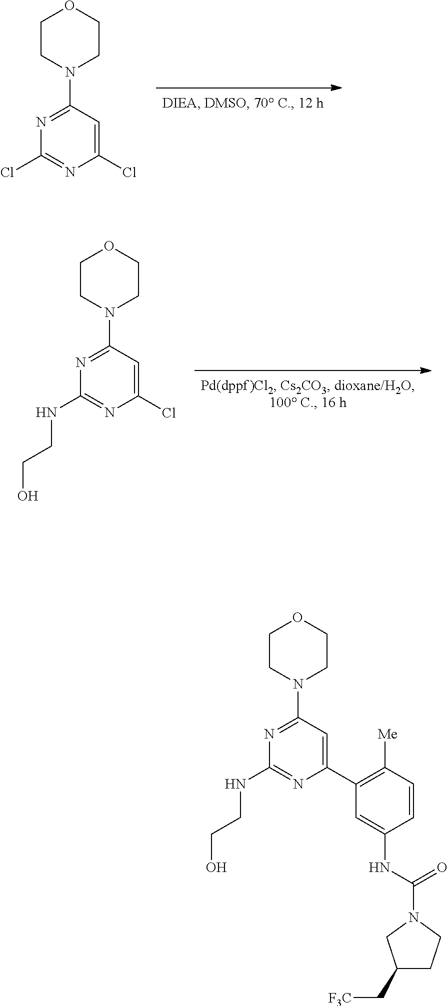 [SMILES: Clc1cc(N2CCOCC2)nc(Cl)n1 ; OCCNc1nc(Cl)cc(N2CCOCC2)n1 ; Cc1ccc(NC(=O)N2CC[C@@H](CC(F)(F)F)C2)cc1-c1cc(N2CCOCC2)nc(NCCO)n1]

Step 1: To a stirred solution of 4-(2,6-dichloropyrimidin-4-yl)morpholine (500 mg, 2.14 mmol) in DMSO (5 mL) were added 2-aminoethanol (170 mg, 2.78 mmol) and DIEA (415 mg, 3.21 mmol) at rt. The reaction mixture was stirred at 70° C. for 12 h. The resulting mixture was diluted with water (10 mL) and extracted with EA (10 mL×3). The combined organic layers was washed with brine (5 mL×4), dried over anhydrous Na2SO4 and filtered. The filtrate was concentrated under reduced pressure. The residue was purified by FCC (PE:EA=2:1) to afford 2-((4-chloro-6-morpholinopyrimidin-2-yl)amino)ethanol (360 mg, 65%) as a white solid. MS Calcd.: 258, MS Found: 259 ([M+H]⁺).
Step 2: To a solution of 2-((4-chloro-6-morpholinopyrimidin-2-yl)amino)ethanol (32 mg, 0.12 mmol), (S)-N-(4-methyl-3-(4,4,5,5-tetramethyl-1,3,2-dioxaborolan-2-yl)phenyl)-3-(2,2,2-trifluoroethyl)pyrrolidine-1-carboxamide (50 mg, 0.12 mmol) and Cs₂CO₃ (98 mg, 0.30 mmol) in dioxane (10 mL) and water (2 mL) was added Pd(dppf)Cl₂ (9 mg, 0.012 mmol) at room temperature under nitrogen atmosphere. The reaction mixture was stirred at 100° C. for 16 h. The mixture was cooled down to room temperature, filtered and concentrated. The residue was purified by prep-HPLC to afford (S)-N-(3-(2-((2-hydroxyethyl)amino)-6-morpholinopyrimidin-4-yl)-4-methylphenyl)-3-(2,2,2-trifluoroethyl)pyrrolidine-1-carboxamide formate (15.8 mg, 25%) as a yellow solid. ¹H NMR (400 MHz, DMSO-d₆): δ 8.17 (s, 1H), 8.12 (s, 1H), 7.45-7.43 (m, 2H), 7.07 (d, J=9.2 Hz, 1H), 6.43 (br s, 1H), 6.02 (s, 1H), 4.62 (br, 0.5H), 3.69-3.64 (m, 5H), 3.54-3.46 (m, 7H), 3.41-3.30 (m, 3H), 3.02 (t, J=9.2 Hz, 1H), 2.46-2.36 (m, 3H), 2.24 (s, 3H), 2.10-2.07 (m, 1H), 1.68-1.63 (m, 1H). MS Calcd.: 508, MS Found: 509 ([M+H]⁺).

### Example 98: (S)-N-(3-(2-(((R)-1-hydroxypropan-2-yl)amino)-6-morpholinopyrimidin-4-yl)-4-methylphenyl)-3-(2,2,2-trifluoroethyl)pyrrolidine-1-carboxamide

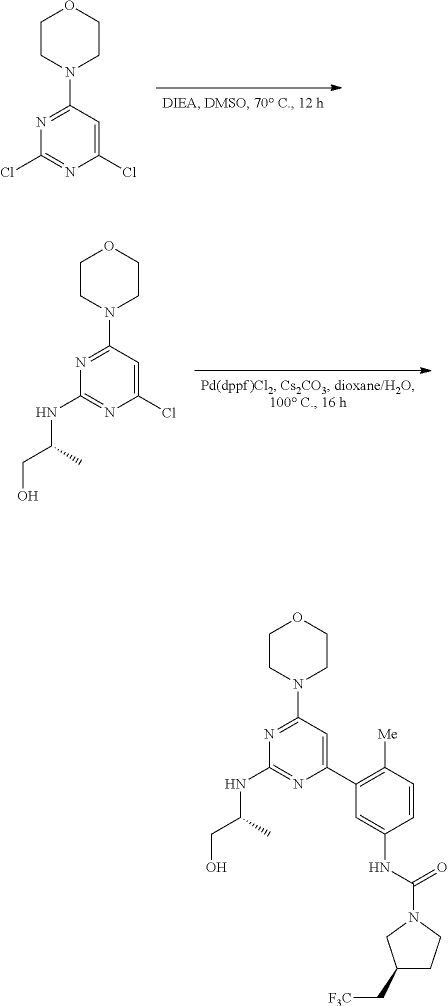 [SMILES: Clc1cc(N2CCOCC2)nc(Cl)n1 ; C[C@H](CO)Nc1nc(Cl)cc(N2CCOCC2)n1 ; Cc1ccc(NC(=O)N2CC[C@@H](CC(F)(F)F)C2)cc1-c1cc(N2CCOCC2)nc(N[C@H](C)CO)n1]

Step 1: To a stirred solution of 4-(2,6-dichloropyrimidin-4-yl)morpholine (500 mg, 2.14 mmol) in DMSO (5 mL) were added (R)-2-aminopropan-1-ol (210 mg, 2.78 mmol) and DIEA (415 mg, 3.21 mmol) at rt. The reaction mixture was stirred at 70° C. for 12 h. The resulting mixture was cooled to rt, diluted with water (10 mL) and extracted with EA (10 mL×3). The combined organic layers was washed with brine (5 mL×4), dried over anhydrous Na₂SO₄ and filtered. The filtrate was concentrated under reduced pressure. The residue was purified by FCC (PE:EA=1:1) to afford (R)-2-((4-chloro-6-morpholinopyrimidin-2-yl)amino)propan-1-ol (180 mg, 31%) as a yellow solid. MS Calcd.: 272, MS Found: 273 ([M+H]⁺).
Step 2: To a solution of (R)-2-((4-chloro-6-morpholinopyrimidin-2-yl)amino)propan-1-ol (32 mg, 0.12 mmol), (S)-N-(4-methyl-3-(4,4,5,5-tetramethyl-1,3,2-dioxaborolan-2-yl)phenyl)-3-(2,2,2-trifluoroethyl)pyrrolidine-1-carboxamide (50 mg, 0.12 mmol) and Cs₂CO₃ (98 mg, 0.30 mmol) in dioxane (10 mL) and water (2 mL) was added Pd(dppf)Cl₂ (9 mg, 0.012 mmol) at room temperature under nitrogen atmosphere. The reaction mixture was stirred at 100° C. for 16 h. The mixture was cooled down to room temperature, filtered and concentrated. The residue was purified by prep-HPLC to afford (S)-N-(3-(2-(((R)-1-hydroxypropan-2-yl)amino)-6-morpholinopyrimidin-4-yl)-4-methylphenyl)-3-(2,2,2-trifluoroethyl)pyrrolidine-1-carboxamide (4.18 mg, 6%) as a pink solid. ¹H NMR (400 MHz, DMSO-d₆): δ 8.12 (s, 1H), 7.46-7.43 (m, 2H), 7.08 (d, J=8.0 Hz, 1H), 6.17-6.15 (m, 1H), 6.02 (s, 1H), 4.64 (br s, 1H), 3.98-3.91 (m, 1H), 3.69-3.59 (m, 5H), 3.54-3.43 (m, 6H), 3.31-3.29 (m, 2H), 3.04-2.98 (m, 1H), 2.46-2.32 (m, 3H), 2.25 (s, 3H), 2.10-2.07 (m, 1H), 1.68-1.63 (m, 1H), 1.11 (d, J=6.8 Hz, 3H). MS Calcd.: 522, MS Found: 523 ([M+H]⁺).

### Example 99: (S)-N-(3-(2-((2-hydroxy-2-methylpropyl)amino)-6-morpholinopyrimidin-4-yl)-4-methylphenyl)-3-(2,2,2-trifluoroethyl)pyrrolidine-1-carboxamide

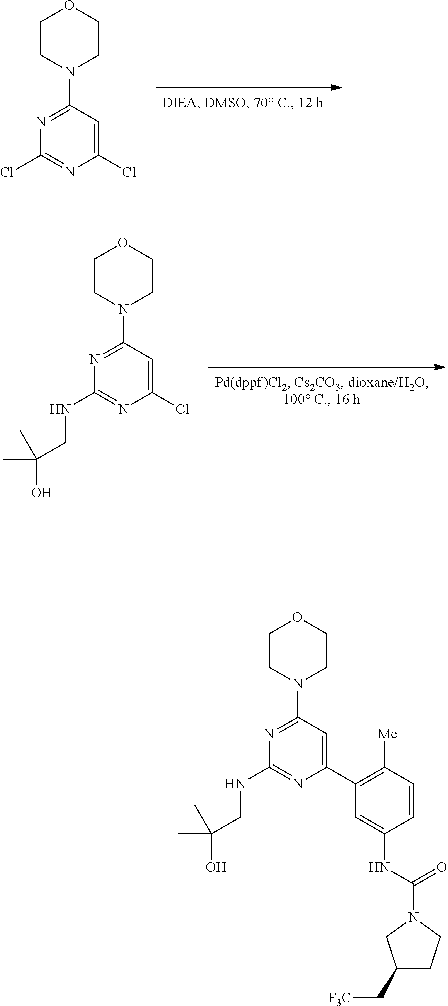 [SMILES: Clc1cc(N2CCOCC2)nc(Cl)n1 ; CC(C)(O)CNc1nc(Cl)cc(N2CCOCC2)n1 ; Cc1ccc(NC(=O)N2CC[C@@H](CC(F)(F)F)C2)cc1-c1cc(N2CCOCC2)nc(NCC(C)(C)O)n1]

Step 1: To a stirred solution of 4-(2,6-dichloropyrimidin-4-yl)morpholine (500 mg, 2.14 mmol) in DMSO (5 mL) were added 1-amino-2-methylpropan-2-ol (250 mg, 2.78 mmol) and DIEA (415 mg, 3.21 mmol) at rt. The reaction mixture was stirred at 70° C. for 12 h. The resulting mixture was diluted with water (10 mL) and extracted with EA (10 mL×3). The combined organic layers was washed with brine (5 mL×4), dried over anhydrous Na₂SO₄ and filtered. The filtrate was concentrated under reduced pressure. The residue was purified by FCC (PE:EA=1:1) to afford 1-((4-chloro-6-morpholinopyrimidin-2-yl)amino)-2-methylpropan-2-ol (280 mg, 46%) as a yellow solid. MS Calcd.: 286, MS Found: 287 ([M+H]⁺).
Step 2: To a solution of 1-((4-chloro-6-morpholinopyrimidin-2-yl)amino)-2-methylpropan-2-ol (39 mg, 0.12 mmol), (S)-N-(4-methyl-3-(4,4,5,5-tetramethyl-1,3,2-dioxaborolan-2-yl)phenyl)-3-(2,2,2-trifluoroethyl)pyrrolidine-1-carboxamide (50 mg, 0.12 mmol) and Cs₂CO₃ (98 mg, 0.30 mmol) in dioxane (10 mL) and water (2 mL) was added Pd(dppf)Cl₂ (9 mg, 0.012 mmol) at room temperature under nitrogen atmosphere. The reaction mixture was stirred at 100° C. for 16 h. The mixture was cooled down to room temperature, filtered and concentrated. The residue was purified by prep-HPLC to afford (S)-N-(3-(2-((2-hydroxy-2-methylpropyl)amino)-6-morpholinopyrimidin-4-yl)-4-methylphenyl)-3-(2,2,2-trifluoroethyl)pyrrolidine-1-carboxamide (14.3 mg, 22%) as a yellow solid. 1H NMR (400 MHz, DMSO-d₆): δ 8.13 (s, 1H), 7.46-7.44 (m, 2H), 7.08 (d, J=9.2 Hz, 1H), 6.26 (br s, 1H), 6.03 (s, 1H), 3.69-3.64 (m, 5H), 3.55-3.50 (m, 5H), 3.30-3.25 (m, 3H), 3.02 (t, J=9.2 Hz, 1H), 2.48-2.38 (m, 3H), 2.24 (s, 3H), 2.10-2.07 (m, 1H), 1.68-1.65 (m, 1H), 1.04 (s, 6H). MS Calcd.: 536, MS Found: 537 ([M+H]⁺).

### Example 100: (S)-N-(3-(6-(((R)-1-hydroxypropan-2-yl)amino)-2-morpholinopyrimidin-4-yl)-4-methylphenyl)-3-(2,2,2-trifluoroethyl)pyrrolidine-1-carboxamide formate

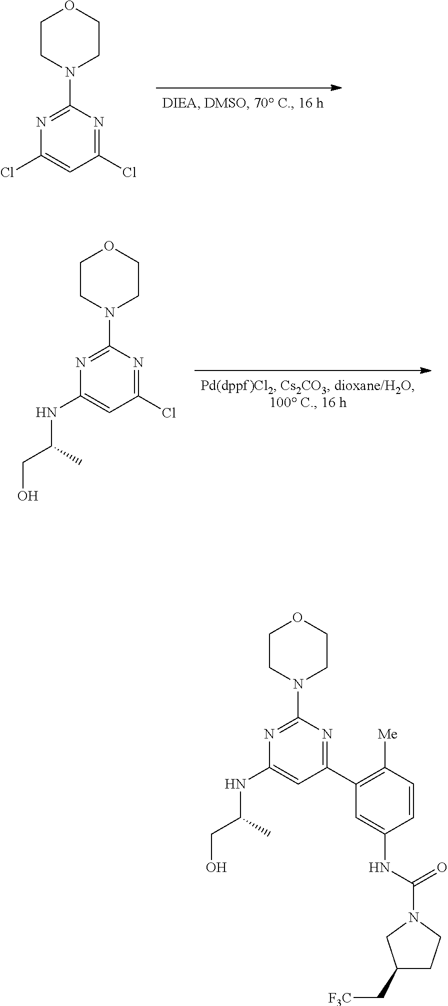 [SMILES: Clc1cc(Cl)nc(N2CCOCC2)n1 ; C[C@H](CO)Nc1cc(Cl)nc(N2CCOCC2)n1 ; Cc1ccc(NC(=O)N2CC[C@@H](CC(F)(F)F)C2)cc1-c1cc(N[C@H](C)CO)nc(N2CCOCC2)n1]

Step 1: To a stirred solution of 4-(4,6-dichloropyrimidin-2-yl)morpholine (300 mg, 1.28 mmol) in DMSO (5 mL) were added (R)-2-aminopropan-1-ol (125 mg, 1.66 mmol) and DIEA (248 mg, 1.92 mmol) at rt. The reaction mixture was stirred at 70° C. for 16 h. The resulting mixture was diluted with water (10 mL) and extracted with EA (10 mL×3). The combined organic layers was washed with brine (5 mL×4), dried over anhydrous Na₂SO₄ and filtered. The filtrate was concentrated under reduced pressure. The residue was purified by FCC (PE:EA=1:1) to afford (R)-2-((6-chloro-2-morpholinopyrimidin-4-yl)amino)propan-1-ol (270 mg, 77%) as a white solid. MS Calcd.: 272, MS Found: 273 ([M+H]⁺).
Step 2: To a solution of (R)-2-((6-chloro-2-morpholinopyrimidin-4-yl)amino)propan-1-ol (66 mg, 0.24 mmol), (S)-N-(4-methyl-3-(4,4,5,5-tetramethyl-1,3,2-dioxaborolan-2-yl)phenyl)-3-(2,2,2-trifluoroethyl)pyrrolidine-1-carboxamide (100 mg, 0.24 mmol) and Cs₂CO₃ (198 mg, 0.61 mmol) in dioxane (10 mL) and water (2 mL) was added Pd(dppf)Cl₂ (9 mg, 0.012 mmol) at room temperature under nitrogen atmosphere. The reaction mixture was stirred at 100° C. for 16 h. The mixture was cooled down to room temperature, filtered and concentrated. The residue was purified by prep-HPLC to afford (S)-N-(3-(6-(((R)-1-hydroxypropan-2-yl)amino)-2-morpholinopyrimidin-4-yl)-4-methylphenyl)-3-(2,2,2-trifluoroethyl)pyrrolidine-1-carboxamide formate (29.4 mg, 23%) as a white solid. ¹H NMR (400 MHz, DMSO-d₆): δ 8.21 (s, 0.3H), 8.12 (s, 1H), 7.50 (d, J=2.4 Hz, 1H), 7.41 (dd, J=2.0, 8.0 Hz, 1H), 7.06 (d, J=4.4 Hz, 1H), 6.82 (br s, 1H), 5.86 (s, 1H), 4.70 (br s, 1H), 3.98 (br s, 1H), 3.69-3.63 (m, 9H), 3.55-3.46 (m, 2H), 3.34-3.27 (m, 2H), 3.02 (t, J=9.2 Hz, 1H), 2.49-2.41 (m, 3H), 2.26 (s, 3H), 2.10-2.07 (m, 1H), 1.68-1.63 (m, 1H), 1.13 (d, J=6.8 Hz, 3H). MS Calcd.: 522, MS Found: 523 ([M+H]⁺).

### Example 101: (S)-N-(3-(2-(((S)-1-hydroxypropan-2-yl)amino)-6-morpholinopyrimidin-4-yl)-4-methylphenyl)-3-(2,2,2-trifluoroethyl)pyrrolidine-1-carboxamide

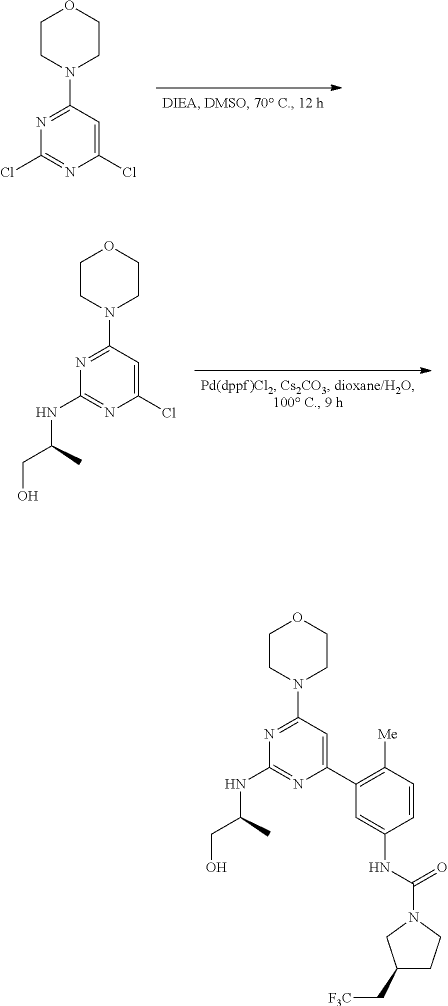 [SMILES: Clc1cc(N2CCOCC2)nc(Cl)n1 ; C[C@@H](CO)Nc1nc(Cl)cc(N2CCOCC2)n1 ; Cc1ccc(NC(=O)N2CC[C@@H](CC(F)(F)F)C2)cc1-c1cc(N2CCOCC2)nc(N[C@@H](C)CO)n1]

Step1: To a stirred solution of 4-(2,6-dichloropyrimidin-4-yl)morpholine (1.0 g, 4.28 mmol) in DMSO (15 mL) were added (S)-2-aminopropan-1-ol (420 mg, 5.56 mmol) and DIEA (830 mg, 6.42 mmol) at rt. The reaction mixture was stirred at 70° C. for 12 h. The resulting mixture was cooled to rt, diluted with water (30 mL) and extracted with EA (15 mL×3). The combined organic layers was washed with brine (15 mL×4), dried over anhydrous Na₂SO₄ and filtered. The filtrate was concentrated under reduced pressure. The residue was purified by FCC (PE:EA=1:1) to afford (S)-2-((4-chloro-6-morpholinopyrimidin-2-yl)amino)propan-1-ol (580 mg, 50%) as a white solid. MS Calcd.: 272, MS Found: 273 ([M+H]⁺).
Step 2: To a solution of (S)-2-((4-chloro-6-morpholinopyrimidin-2-yl)amino)propan-1-ol (50 mg, 0.18 mmol), (S)-N-(4-methyl-3-(4,4,5,5-tetramethyl-1,3,2-dioxaborolan-2-yl)phenyl)-3-(2,2,2-trifluoroethyl)pyrrolidine-1-carboxamide (75 mg, 0.18 mmol) and Cs₂CO₃ (150 mg, 0.46 mmol) in dioxane (10 mL) and water (2 mL) was added Pd(dppf)Cl₂ (27 mg, 0.036 mmol) at room temperature under nitrogen atmosphere. The reaction mixture was stirred at 100° C. for 9 h. The mixture was cooled down to room temperature, filtered and concentrated. The residue was purified by prep-HPLC to afford (S)-N-(3-(2-(((R)-1-hydroxypropan-2-yl)amino)-6-morpholinopyrimidin-4-yl)-4-methylphenyl)-3-(2,2,2-trifluoroethyl)pyrrolidine-1-carboxamide (7.3 mg, 7.6%) as a white solid. ¹H NMR (400 MHz, DMSO-d₆): δ 8.12 (s, 1H), 7.46-7.43 (m, 2H), 7.07 (d, J=8.4 Hz, 1H), 6.16 (br s, 1H), 6.01 (s, 1H), 4.63-4.62 (m, 1H), 3.92-3.91 (m, 1H), 3.69-3.64 (m, 5H), 3.59-3.43 (m, 6H), 3.31-3.24 (m, 2H), 3.02 (t, J=9.2 Hz, 1H), 2.46-2.32 (m, 3H), 2.25 (s, 3H), 2.11-2.07 (m, 1H), 1.70-1.60 (m, 1H), 1.11 (d, J=6.4 Hz, 3H). MS Calcd.: 522, MS Found: 523 ([M+H]⁺).

### Examples 102 & 103: (R)-N-(3-(2-((S)-3-hydroxypyrrolidin-1-yl)-6-morpholinopyridin-4-yl)-4-methylphenyl)-3-(2,2,2-trifluoroethyl)pyrrolidine-1-carboxamide & (S)-N-(3-(2-((S)-3-hydroxypyrrolidin-1-yl)-6-morpholinopyridin-4-yl)-4-methylphenyl)-3-(2,2,2-trifluoroethyl)pyrrolidine-1-carboxamide

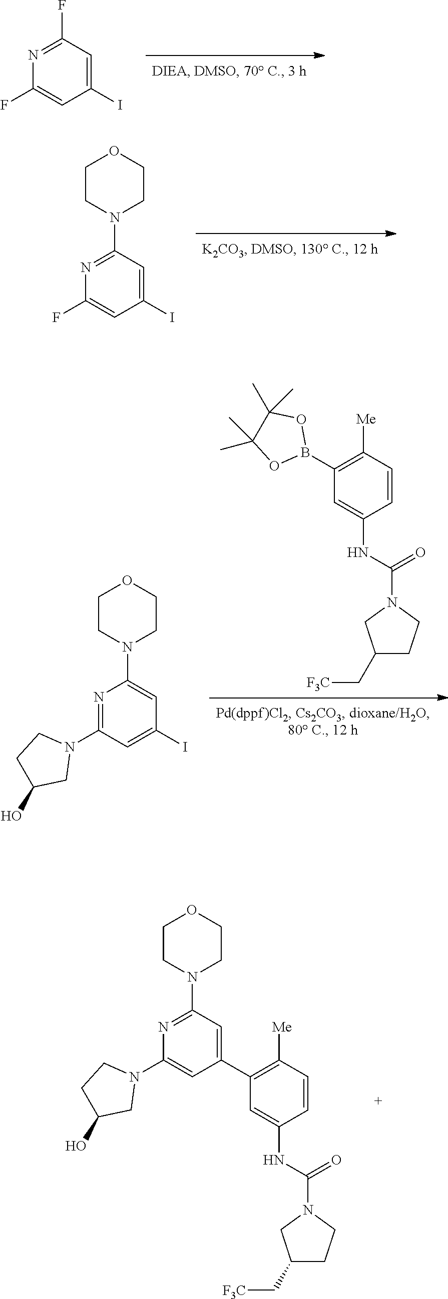

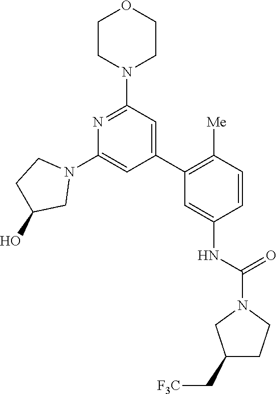 [SMILES: Cc1ccc(NC(=O)N2CC[C@@H](CC(F)(F)F)C2)cc1-c1cc(N2CCOCC2)nc(N2CC[C@H](O)C2)c1]

Step 1: To a stirred solution of 2,6-difluoro-4-iodopyridine (16.00 g, 66.40 mmol) in DMSO (240 mL) were added morpholine (5.49 mL, 63.04 mmol) and DIEA (12.07 mL, 93.40 mmol) at room temperature under nitrogen atmosphere. The reaction mixture was stirred at 70° C. for 3 h. The resulting mixture was cooled to rt, diluted with water (150 mL) and extracted with EA (300 mL×3). The combined organic layers was washed with brine (100 mL×4), dried over anhydrous Na₂SO₄, filtered and concentrated under reduced pressure. The residue was purified by silica gel column chromatography eluting with 30% EA in PE to afford 4-(6-fluoro-4-iodopyridin-2-yl)morpholine (17.60 g, 86%) as an off-white solid. MS Calcd.: 308, MS Found: 309 ([M+H]⁺).
Step 2: To a stirred solution of 4-(6-fluoro-4-iodopyridin-2-yl)morpholine (600 mg, 1.95 mmol) in DMSO (15 mL) was added (S)-pyrrolidin-3-ol (254 mg, 2.92 mmol) and K₂CO₃ (538 mg, 3.89 mmol) at rt. The reaction mixture was stirred at 130° C. for 12 h. The resulting mixture was cooled to rt, diluted with water (30 mL) and extracted with EA (15 mL×3). The combined organic layers was washed with brine (15 mL×4), dried over anhydrous Na₂SO₄ and filtered. The filtrate was concentrated under reduced pressure. The residue was purified by FCC (PE:EA=5:1) to afford (S)-1-(4-iodo-6-morpholinopyridin-2-yl)pyrrolidin-3-ol (500 mg, 68%) as a white solid. MS Calcd.: 375, MS Found: 376 ([M+H]⁺).
Step 3: To a solution of (S)-1-(4-iodo-6-morpholinopyridin-2-yl)pyrrolidin-3-ol (300 mg, 0.80 mmol), N-(4-methyl-3-(4,4,5,5-tetramethyl-1,3,2-dioxaborolan-2-yl)phenyl)-3-(2,2,2-trifluoroethyl)pyrrolidine-1-carboxamide (330 mg, 0.80 mmol) and Cs₂CO₃ (650 mg, 1.99 mmol) in dioxane (40 mL) and water (4 mL) was added Pd(dppf)Cl₂ (60 mg, 0.08 mmol) at room temperature under nitrogen atmosphere. The reaction mixture was stirred at 80° C. for 12 h. The mixture was cooled down to room temperature, filtered and concentrated. The residue was purified by chiral prep-SFC (Column: Chiralpak IC 5 μm*20*250 mm; Mobile Phase: CO₂: MeOH=65: 35; Temp: 40° C.; Wavelength: 214 nm, Back pressure: 100 bar) to afford (R)-N-(3-(2-((S)-3-hydroxypyrrolidin-1-yl)-6-morpholinopyridin-4-yl)-4-methylphenyl)-3-(2,2,2-trifluoroethyl)pyrrolidine-1-carboxamide (111.4 mg, 26%, RT=7.31 min, ee>98%) and (S)-N-(3-(2-((S)-3-hydroxypyrrolidin-1-yl)-6-morpholinopyridin-4-yl)-4-methylphenyl)-3-(2,2,2-trifluoroethyl)pyrrolidine-1-carboxamide (114.6 mg, 27%, RT=6.45 min, ee>98%) as a white solid. (R)-N-(3-(2-((S)-3-hydroxypyrrolidin-1-yl)-6-morpholinopyridin-4-yl)-4-methylphenyl)-3-(2,2,2-trifluoroethyl)pyrrolidine-1-carboxamide: ¹H NMR (400 MHz, DMSO-d₆): δ 8.10 (s, 1H), 7.42 (dd, J=2.4, 8.4 Hz, 1H), 7.33 (d, J=2.0 Hz, 1H), 7.10 (d, J=8.4 Hz, 1H), 5.83 (s, 1H), 5.63 (s, 1H), 4.88 (d, J=4.0 Hz, 1H), 4.35 (br s, 1H), 3.68-3.64 (m, 5H), 3.54-3.39 (m, 8H), 3.29-3.27 (m, 2H), 3.02 (t, J=9.2 Hz, 1H), 2.49-2.36 (m, 3H), 2.16 (s, 3H), 2.09-1.95 (m, 2H), 1.87-1.85 (m, 1H), 1.68-1.63 (m, 1H). (S)-N-(3-(2-((S)-3-hydroxypyrrolidin-1-yl)-6-morpholinopyridin-4-yl)-4-methylphenyl)-3-(2,2,2-trifluoroethyl)pyrrolidine-1-carboxamide: ¹H NMR (400 MHz, DMSO-d₆): δ 8.10 (s, 1H), 7.42 (dd, J=2.4, 8.4 Hz, 1H), 7.33 (d, J=2.4 Hz, 1H), 7.10 (d, J=8.4 Hz, 1H), 5.83 (s, 1H), 5.63 (s, 1H), 4.88 (d, J=3.6 Hz, 1H), 4.35 (br s, 1H), 3.69-3.64 (m, 5H), 3.54-3.39 (m, 8H), 3.29-3.27 (m, 2H), 3.02 (t, J=9.2 Hz, 1H), 2.49-2.37 (m, 3H), 2.16 (s, 3H), 2.11-1.96 (m, 2H), 1.87-1.85 (m, 1H), 1.68-1.62 (m, 1H). MS Calcd.: 533, MS Found: 534 ([M+H]⁺).

### Example 104: (S)-N-(3-(2-(((S)-2-hydroxypropyl)amino)-6-morpholinopyrimidin-4-yl)-4-methylphenyl)-3-(2,2,2-trifluoroethyl)pyrrolidine-1-carboxamide

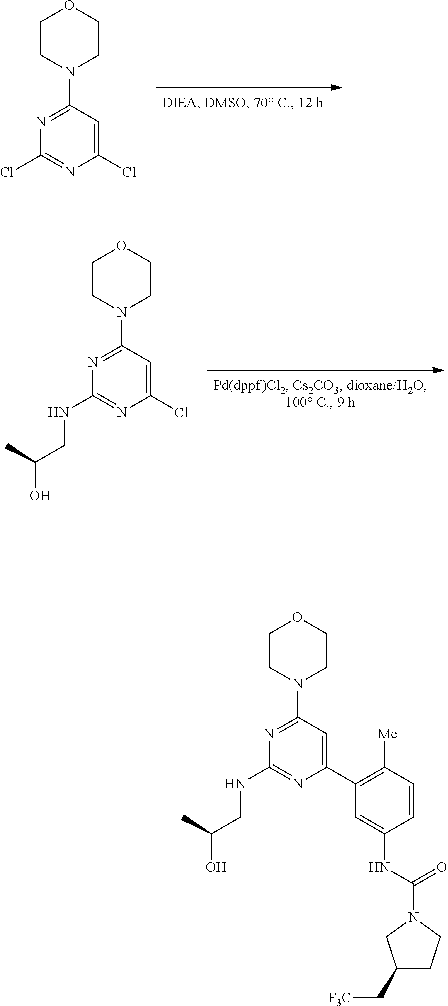 [SMILES: Clc1cc(N2CCOCC2)nc(Cl)n1 ; C[C@H](O)CNc1nc(Cl)cc(N2CCOCC2)n1 ; Cc1ccc(NC(=O)N2CC[C@@H](CC(F)(F)F)C2)cc1-c1cc(N2CCOCC2)nc(NC[C@H](C)O)n1]

Step 1: To a stirred solution of 4-(2,6-dichloropyrimidin-4-yl)morpholine (500 mg, 2.14 mmol) in DMSO (15 mL) were added (S)-1-aminopropan-2-ol (210 mg, 2.78 mmol) and DIEA (400 mg, 3.21 mmol) at rt. The reaction mixture was stirred at 70° C. for 12 h. The resulting mixture was cooled to rt, diluted with water (30 mL) and extracted with EA (15 mL×3). The combined organic layers were washed with brine (15 mL×4), dried over anhydrous Na₂SO₄ and filtered. The filtrate was concentrated under reduced pressure. The residue was purified by FCC (PE:EA=1:1) to afford (S)-1-((4-chloro-6-morpholinopyrimidin-2-yl)amino)propan-2-ol (260 mg, 45%) as a white solid. MS Calcd.: 272, MS Found: 273 ([M+H]⁺).
Step 2: To a solution of (S)-1-((4-chloro-6-morpholinopyrimidin-2-yl)amino)propan-2-ol (50 mg, 0.18 mmol), (S)-N-(4-methyl-3-(4,4,5,5-tetramethyl-1,3,2-dioxaborolan-2-yl)phenyl)-3-(2,2,2-trifluoroethyl)pyrrolidine-1-carboxamide (75 mg, 0.18 mmol) and Cs₂CO₃ (150 mg, 0.46 mmol) in dioxane (10 mL) and water (2 mL) was added Pd(dppf)Cl₂ (27 mg, 0.036 mmol) at room temperature under nitrogen atmosphere. The reaction mixture was stirred at 100° C. for 9 h. The mixture was cooled down to room temperature, filtered and concentrated. The residue was purified by prep-HPLC to afford (S)-N-(3-(2-(((S)-2-hydroxypropyl)amino)-6-morpholinopyrimidin-4-yl)-4-methylphenyl)-3-(2,2,2-trifluoroethyl)pyrrolidine-1-carboxamide (7.6 mg, 8%) as a white solid. ¹H NMR (400 MHz, DMSO-d₆): δ 8.12 (s, 1H), 7.45-7.43 (m, 2H), 7.09-7.06 (m, 1H), 6.40-6.37 (m, 1H), 6.02 (s, 1H), 4.74-4.70 (m, 1H), 3.78-3.76 (m, 1H), 3.69-3.64 (m, 5H), 3.55-3.50 (m, 5H), 3.26-3.14 (m, 3H), 3.02 (t, J=9.2 Hz, 1H), 2.46-2.38 (m, 3H), 2.24 (s, 3H), 2.10-2.04 (m, 1H), 1.70-1.60 (m, 1H), 1.04 (d, J=6.0 Hz, 3H). MS Calcd.: 522, MS Found: 523 ([M+H]⁺).

### Example 105: (3S)-N-(3-[2-[(3R)-3-aminopyrrolidin-1-yl]-6-(morpholin-4-yl)pyridin-4-yl]-4-methylphenyl)-3-(2,2,2-trifluoroethyl)pyrrolidine-1-carboxamide

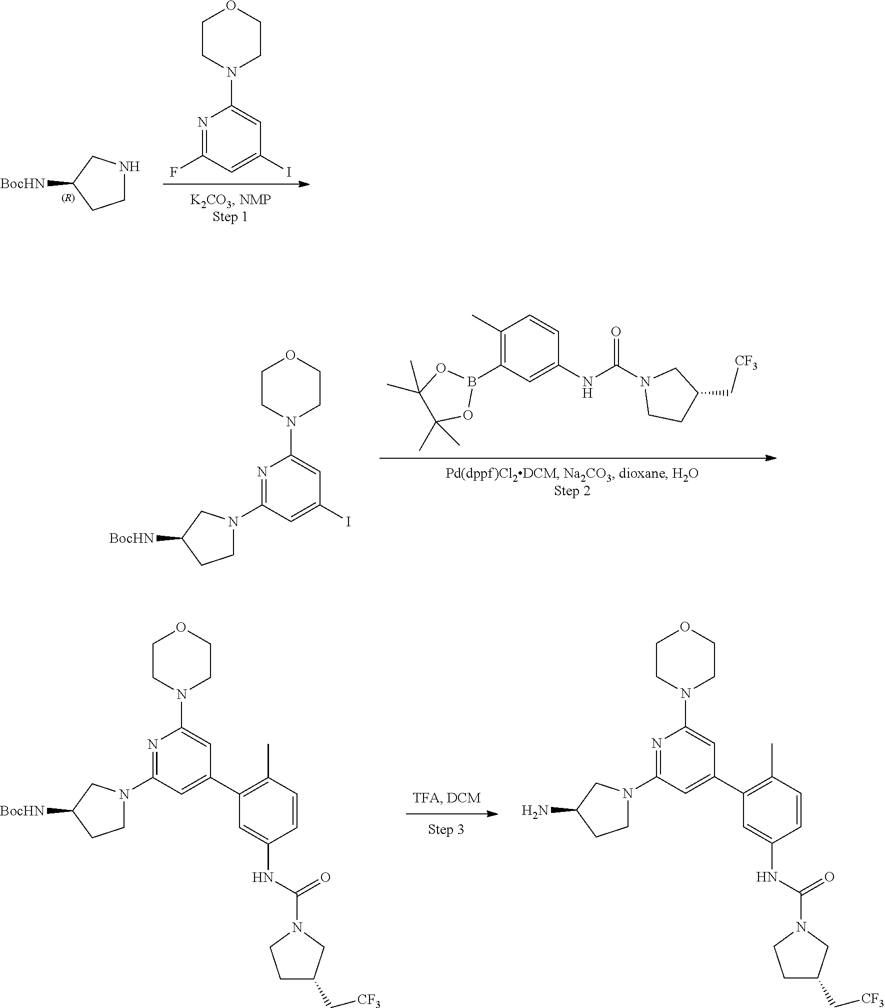

Preparation 105A: tert-butyl N-[(3R)-1-[4-iodo-6-(morpholin-4-yl)pyridin-2-yl]pyrrolidin-3-yl]carbamate

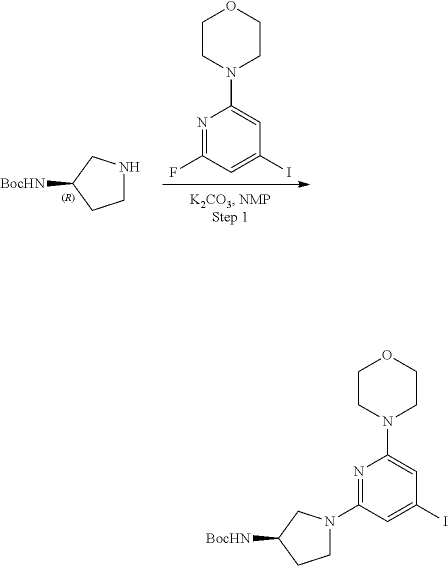

A mixture of 4-(6-fluoro-4-iodopyridin-2-yl)morpholine (500 mg, 1.623 mmol), tert-butyl N-[(3R)-pyrrolidin-3-yl]carbamate (453 mg, 2.434 mmol) and K₂CO₃ (449 mg, 3.246 mmol9) in NMP (5 mL) was stirred at 150° C. for 2 h. The reaction was quenched by the addition of water (100 mL). The resulting mixture was extracted with EtOAc (3×50 mL). The combined organic layers were washed with brine (3×20 mL), dried over anhydrous Na₂SO₄. After filtration, the filtrate was concentrated under reduced pressure. The residue was purified by silica gel column chromatography, eluted with PE/EtOAc (5/1) to afford tert-butyl N-[(3R)-1-[4-iodo-6-(morpholin-4-yl)pyridin-2-yl]pyrrolidin-3-yl]carbamate (588 mg, 76%) as a white solid. MS ESI calculated for C₁₈H₂₇IN₄O₃ [M+H]⁺, 475.11; found 475.10. ¹H NMR (400 MHz, Chloroform-d) δ 6.28 (d, J=1.2 Hz, 1H), 6.16 (d, J=1.2 Hz, 1H), 4.71-4.67 (m, 1H), 4.35-4.25 (m, 1H), 3.82-3.76 (m, 4H), 3.70-3.68 (m, 1H), 3.55-3.44 (m, 6H), 3.34-3.24 (m, 1H), 2.24-2.20 (m, 1H), 1.93-1.91 (m, 1H), 1.47 (s, 9H).

Preparation 105B: tert-butyl N-[(3S)-1-(4-[2-methyl-5-[(3S)-3-(2,2,2-trifluoroethyl)pyrrolidine-1-carbonylamino]phenyl]-6-(morpholin-4-yl)pyridin-2-yl)pyrrolidin-3-yl]carbamate

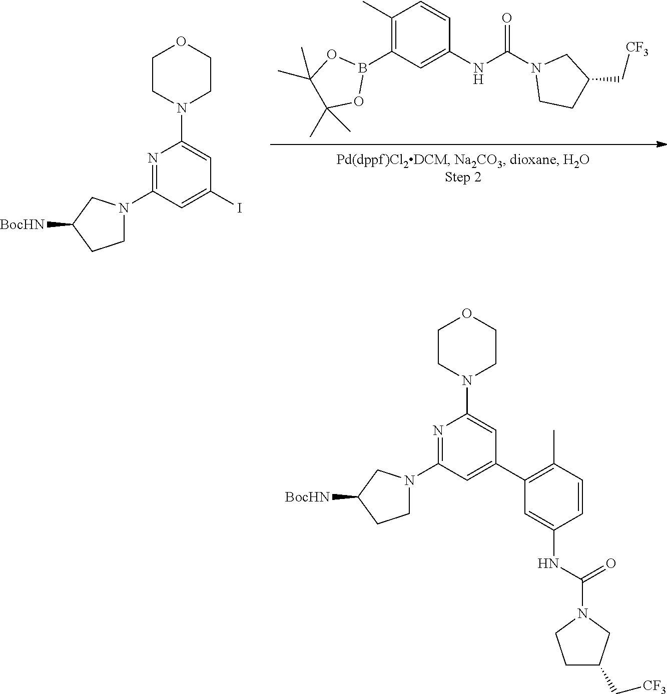 [SMILES: CC(C)(C)OC(=O)N[C@@H]1CCN(c2cc(I)cc(N3CCOCC3)n2)C1 ; Cc1ccc(NC(=O)N2CC[C@@H](CC(F)(F)F)C2)cc1B1OC(C)(C)C(C)(C)O1 ; Cc1ccc(NC(=O)N2CC[C@@H](CC(F)(F)F)C2)cc1-c1cc(N2CCOCC2)nc(N2CC[C@@H](NC(=O)OC(C)(C)C)C2)c1]

A mixture of tert-butyl N-[(3S)-1-[4-iodo-6-(morpholin-4-yl)pyridin-2-yl]pyrrolidin-3-yl]carbamate (173 mg, 0.364 mmol), (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (150 mg, 0.364 mmol), Pd(dppf)Cl₂·CH₂Cl₂ (30 mg, 0.036 mmol) and Na₂CO₃ (116 mg, 1.092 mmol) in dioxane (4 mL) and H₂O (1 mL) was stirred at 60° C. for 16 h under nitrogen atmosphere. The mixture was allowed to cool down to room temperature. The reaction was quenched by the addition of water (100 mL). The resulting mixture was extracted with EtOAc (3×50 mL). The combined organic layers were washed with brine (3×20 mL), dried over anhydrous Na₂SO₄. After filtration, the filtrate was concentrated under reduced pressure. The residue was purified by Prep-TLC with PE/EtOAc (1/1) to afford tert-butyl N-[(3S)-1-(4-[2-methyl-5-[(3S)-3-(2,2,2-trifluoroethyl)pyrrolidine-1-carbonylamino]phenyl]-6-(morpholin-4-yl)pyridin-2-yl)pyrrolidin-3-yl]carbamate (200 mg, 87%) as a light yellow solid. MS ESI calculated for C₃₂H₄₃F₃N₆O₄[M+H]⁺, 633.33, found 633.10. ¹H NMR (400 MHz, Chloroform-d) δ 7.42-7.41 (m, 1H), 7.19-7.17 (m, 2H), 6.14 (s, 1H), 5.87 (s, 1H), 5.73 (s, 1H), 4.77-4.73 (m, 1H), 4.34-4.32 (m, 1H), 3.85-3.80 (m, 5H), 3.74-3.72 (m, 1H), 3.66-3.63 (m, 1H), 3.57-3.45 (m, 7H), 3.38-3.36 (m, 1H), 3.13-3.11 (m, 1H), 2.56-2.52 (m, 1H), 2.31-2.21 (m, 7H), 1.95-1.93 m, 1H), 1.79-1.71 (m, 1H), 1.47 (s, 9H).

### Example 105: (3S)-N-(3-[2-[(3R)-3-aminopyrrolidin-1-yl]-6-(morpholin-4-yl)pyridin-4-yl]-4-methylphenyl)-3-(2,2,2-trifluoroethyl)pyrrolidine-1-carboxamide

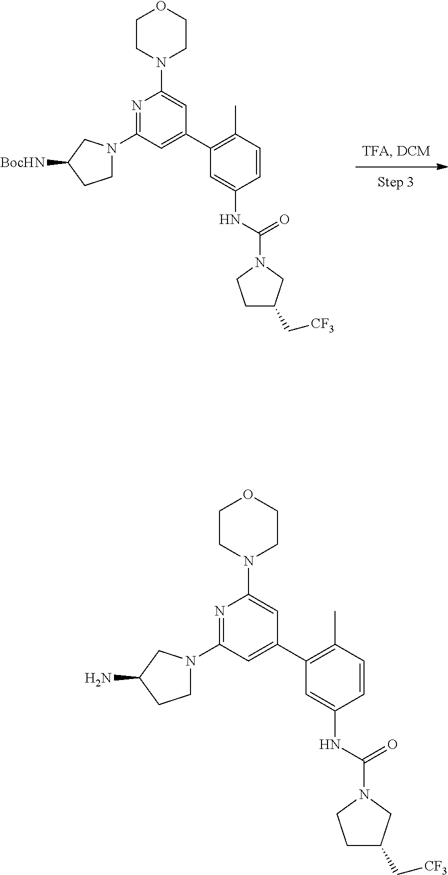 [SMILES: Cc1ccc(NC(=O)N2CC[C@@H](CC(F)(F)F)C2)cc1-c1cc(N2CCOCC2)nc(N2CC[C@@H](NC(=O)OC(C)(C)C)C2)c1 ; Cc1ccc(NC(=O)N2CC[C@@H](CC(F)(F)F)C2)cc1-c1cc(N2CCOCC2)nc(N2CC[C@@H](N)C2)c1]

A mixture of tert-butyl N-[(3R)-1-(4-[2-methyl-5-[(3S)-3-(2,2,2-trifluoroethyl)pyrrolidine-1-carbonylamino]phenyl]-6-(morpholin-4-yl)pyridin-2-yl)pyrrolidin-3-yl]carbamate (233 mg, 0.368 mmol) and TFA (1 mL) in DCM (5 mL) was stirred for 1 h at room temperature. The reaction mixture was concentrated under reduced pressure and basified to pH 8 with saturated NaHCO₃.

The resulting mixture was extracted with EtOAc (3×50 mL). The combined organic layers were washed with brine (3×20 mL), dried over anhydrous Na₂SO₄. After filtration, the filtrate was concentrated under reduced pressure. The residue was purified by reverse flash chromatography with the following conditions: Column: C18 Column 120 g; Mobile Phase A: water (10 mmol/L NH₄HCO₃), Mobile Phase B: CH₃CN; Flow rate: 60 mL/min; Gradient: 30% B to 50% B; 254/220 nm to afford (3S)-N-(3-[2-[(3R)-3-aminopyrrolidin-1-yl]-6-(morpholin-4-yl)pyridin-4-yl]-4-methylphenyl)-3-(2,2,2-trifluoroethyl)pyrrolidine-1-carboxamide (94 mg, 48%) as a white solid. MS ESI calculated for C₂₇H₃₅F₃N₆O₂ [M+H]⁺, 533.28, found 533.40. ¹H NMR (400 MHz, DMSO-d₆) δ 8.12 (s, 1H), 7.43 (dd, J=8.4, 2.4 Hz, 1H), 7.34 (d, J=2.4 Hz, 1H), 7.10 (d, J=8.4 Hz, 1H), 5.83 (s, 1H), 5.61 (s, 1H), 3.72-3.63 (m, 5H), 3.57-3.45 (m, 4H), 3.42-3.26 (m, 6H), 3.03-3.00 (m, 2H), 2.48-2.35 (m, 3H), 2.17 (s, 3H), 2.05-2.00 (m, 2H), 1.84-1.58 (m, 4H). ¹⁹F NMR (376 MHz, DMSO-d₆) δ-63.37 (3F).

### Example 106: (3S)-N-(3-[2-[(3S)-3-aminopyrrolidin-1-yl]-6-(morpholin-4-yl)pyridin-4-yl]-4-methylphenyl)-3-(2,2,2-trifluoroethyl)pyrrolidine-1-carboxamide

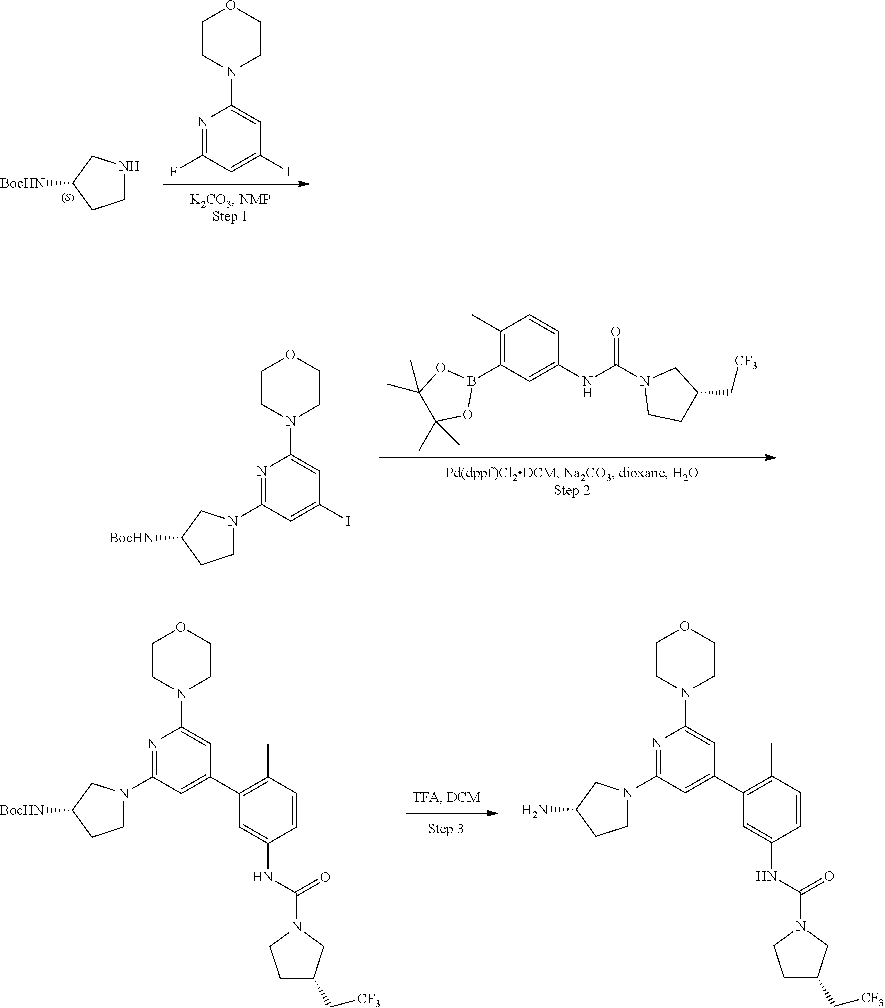

Preparation 106A: tert-butyl N-[(3S)-1-[4-iodo-6-(morpholin-4-yl)pyridin-2-yl]pyrrolidin-3-yl]carbamate

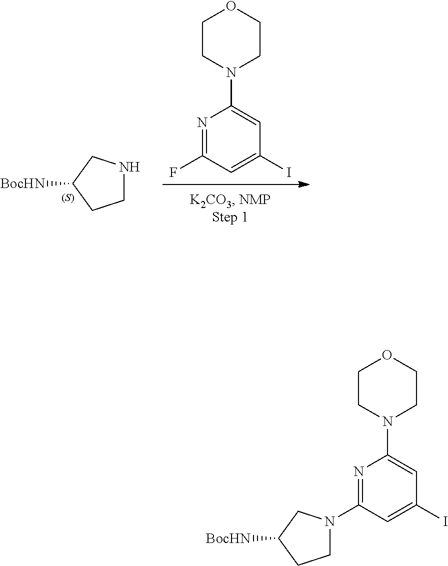

A mixture of 4-(6-fluoro-4-iodopyridin-2-yl)morpholine (500 mg, 1.623 mmol), tert-butyl N-[(3S)-pyrrolidin-3-yl]carbamate (453 mg, 2.434 mmol) and K₂CO₃ (449 mg, 3.246 mmol) in NMP (5 mL) was stirred at 150° C. for 2 h. The reaction was quenched by the addition of water (100 mL) at room temperature. The resulting mixture was extracted with EtOAc (3×50 mL). The combined organic layers were washed with brine (3×20 mL), dried over anhydrous Na₂SO₄. After filtration, the filtrate was concentrated under reduced pressure. The residue was purified by silica gel column chromatography, eluted with PE/EtOAc (5/1) to afford tert-butyl N-[(3S)-1-[4-iodo-6-(morpholin-4-yl)pyridin-2-yl]pyrrolidin-3-yl]carbamate (577 mg, 75%) as a white solid. MS ESI calculated for C₁₈H₂₇IN₄O₃[M+H]⁺, 475.11, found 475.15. ¹H NMR (400 MHz, Chloroform-d) δ 6.28 (d, J=1.2 Hz, 1H), 6.16 (d, J=1.2 Hz, 1H), 4.71-4.67 (s, 1H), 4.35-4.25 (m, 1H), 3.82-3.76 (m, 4H), 3.70-3.68 (m, 1H), 3.55-3.48 (m, 1H), 3.48-3.44 (m, 4H), 3.34-3.24 (m, 1H), 2.24-2.20 (m, 1H), 1.93-1.91 (m, 1H), 1.47 (s, 9H).

Preparation 106B: tert-butyl N-[(3S)-1-(4-[2-methyl-5-[(3S)-3-(2,2,2-trifluoroethyl)pyrrolidine-1-carbonylamino]phenyl]-6-(morpholin-4-yl)pyridin-2-yl)pyrrolidin-3-yl]carbamate

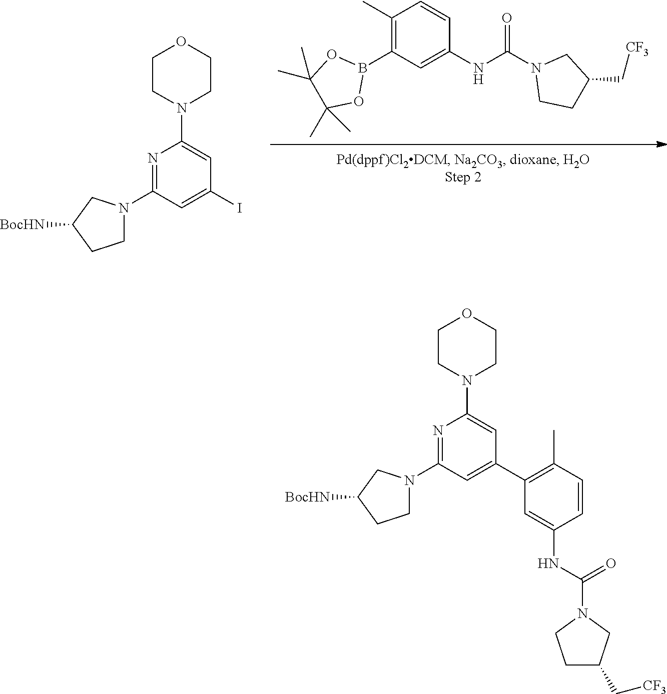 [SMILES: CC(C)(C)OC(=O)N[C@H]1CCN(c2cc(I)cc(N3CCOCC3)n2)C1 ; Cc1ccc(NC(=O)N2CC[C@@H](CC(F)(F)F)C2)cc1B1OC(C)(C)C(C)(C)O1 ; Cc1ccc(NC(=O)N2CC[C@@H](CC(F)(F)F)C2)cc1-c1cc(N2CCOCC2)nc(N2CC[C@H](NC(=O)OC(C)(C)C)C2)c1]

A mixture of tert-butyl N-[(3S)-1-[4-iodo-6-(morpholin-4-yl)pyridin-2-yl]pyrrolidin-3-yl]carbamate (173 mg, 0.364 mmol), (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (150 mg, 0.364 mmol), Pd(dppf)Cl₂·CH₂Cl₂ (30 mg, 0.036 mmol) and Na₂CO₃ (116 mg, 1.092 mmol) in dioxane (4 mL) and H₂O (1 mL) was stirred at 60° C. for 3 h under nitrogen atmosphere. The resulting mixture was concentrated under reduced pressure. The residue was purified by Prep-TLC with PE/EtOAc (1/1) to afford tert-butyl N-[(3S)-1-(4-[2-methyl-5-[(3S)-3-(2,2,2-trifluoroethyl)pyrrolidine-1-carbonylamino]phenyl]-6-(morpholin-4-yl)pyridin-2-yl)pyrrolidin-3-yl]carbamate (200 mg, 87%) as a light yellow solid. MS ESI calculated for C₃₂H₄₃F₃N₆O₄[M+H]⁺, 633.33, found 633.10. ¹H NMR (400 MHz, Chloroform-d) δ 7.42-7.41 (m, 1H), 7.19-7.17 (m, 2H), 6.14 (s, 1H), 5.87 (s, 1H), 5.73 (s, 1H), 4.77-4.73 (m, 1H), 4.34-4.32 (m, 1H), 3.85-3.80 (m, 5H), 3.74-3.72 (m, 1H), 3.66-3.63 (m, 1H), 3.57-3.45 (m, 6H), 3.38-3.36 (m, 2H), 3.13-3.11 (m, 1H), 2.56-2.52 (m, 1H), 2.31-2.21 (m, 7H), 1.95-1.93 m, 1H), 1.79-1.71 (m, 1H), 1.47 (s, 9H).

### Example 106: (3S)-N-(3-[2-[(3S)-3-aminopyrrolidin-1-yl]-6-(morpholin-4-yl)pyridin-4-yl]-4-methylphenyl)-3-(2,2,2-trifluoroethyl)pyrrolidine-1-carboxamide

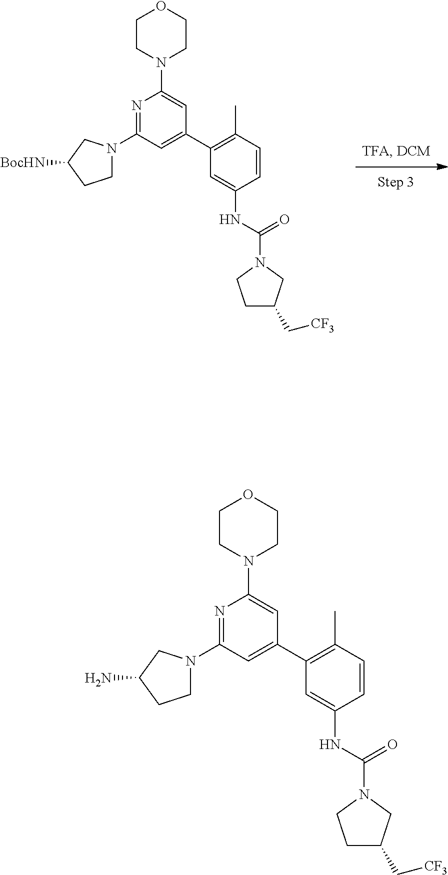 [SMILES: Cc1ccc(NC(=O)N2CC[C@@H](CC(F)(F)F)C2)cc1-c1cc(N2CCOCC2)nc(N2CC[C@H](NC(=O)OC(C)(C)C)C2)c1 ; Cc1ccc(NC(=O)N2CC[C@@H](CC(F)(F)F)C2)cc1-c1cc(N2CCOCC2)nc(N2CC[C@H](N)C2)c1]

A mixture of tert-butyl N-[(3S)-1-(4-[2-methyl-5-[(3S)-3-(2,2,2-trifluoroethyl)pyrrolidine-1-carbonylamino]phenyl]-6-(morpholin-4-yl)pyridin-2-yl)pyrrolidin-3-yl]carbamate (223 mg, 0.368 mmol) and TFA (1 mL) in DCM (5 mL) was stirred for 1 h at room temperature. The reaction mixture was concentrated under reduced pressure and basified to pH 8 with saturated NaHCO₃. The resulting mixture was extracted with EtOAc (50 mL). The combined organic layers were washed with brine (3×20 mL), dried over anhydrous Na₂SO₄. After filtration, the filtrate was concentrated under reduced pressure. The residue was purified by reverse flash chromatography with the following conditions: Column: C18 Column 120 g; Mobile Phase A: water(10 mmol/L NH₄HCO₃), Mobile Phase B: CH₃CN; Flow rate: 60 mL/min; Gradient: 30% B to 50% B in 25 min; 254/220 nm to afford (3S)-N-(3-[2-[(3S)-3-aminopyrrolidin-1-yl]-6-(morpholin-4-yl)pyridin-4-yl]-4-methylphenyl)-3-(2,2,2-trifluoroethyl)pyrrolidine-1-carboxamide (81 mg, 48%) as a white solid. MS ESI calculated for C₂₇H₃₅F₃N₆O₂ [M+H]⁺, 533.28, found 533.15. ¹H NMR (400 MHz, DMSO-d₆) δ 8.12 (s, 1H), 7.43 (dd, J=8.4, 2.4 Hz, 1H), 7.34 (d, J=2.4 Hz, 1H), 7.10 (d, J=8.4 Hz, 1H), 5.83 (s, 1H), 5.61 (s, 1H), 3.72-3.63 (m, 5H), 3.57-3.45 (m, 4H), 3.42-3.26 (m, 6H), 3.03-3.00 (m, 2H), 2.48-2.35 (m, 3H), 2.17 (s, 3H), 2.05-2.00 (m, 2H), 1.94-1.60 (m, 4H). ¹⁹F NMR (376 MHz, DMSO-d₆) δ-63.37 (3F).

### Example 107: (3S)-N-(3-[2-[(3R)-3-aminopiperidin-1-yl]-6-(morpholin-4-yl)pyridin-4-yl]-4-methylphenyl)-3-(2,2,2-trifluoroethyl)pyrrolidine-1-carboxamide

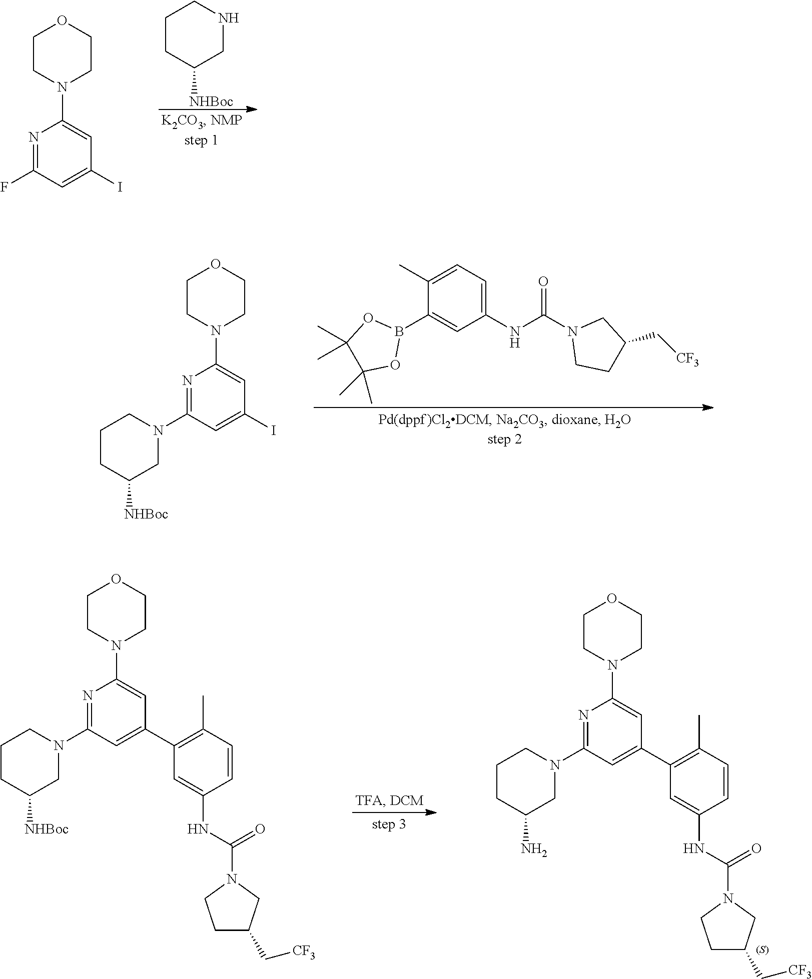

Preparation 107A: tert-butyl N-[(3R)-1-[4-iodo-6-(morpholin-4-yl)pyridin-2-yl]piperidin-3-yl]carbamate

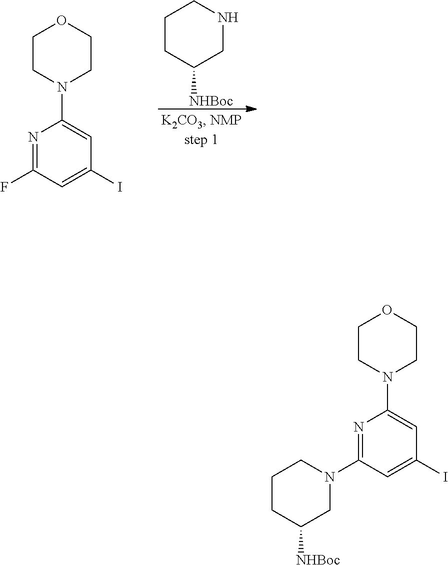 [SMILES: Fc1cc(I)cc(N2CCOCC2)n1 ; CC(C)(C)OC(=O)N[C@@H]1CCCNC1 ; CC(C)(C)OC(=O)N[C@@H]1CCCN(c2cc(I)cc(N3CCOCC3)n2)C1]

A mixture of 4-(6-fluoro-4-iodopyridin-2-yl)morpholine (500 mg, 1.623 mmol), tert-butyl N-[(3R)-piperidin-3-yl]carbamate (486 mg, 2.435 mmol) in NMP (5 mL) and K₂CO₃ (449 mg, 3.246 mmol) was stirred for 2 h at 150° C. The reaction was quenched by the addition of water (30 mL). The resulting mixture was extracted with EtOAc (3×40 mL). The combined organic layers were washed with brine (30 mL), dried over anhydrous Na₂SO₄. After filtration, the filtrate was concentrated under reduced pressure.The residue was purified by silica gel column chromatography, eluted with PE/EtOAc (3/1) to afford tert-butyl N-[(3R)-1-[4-iodo-6-(morpholin-4-yl)pyridin-2-yl]piperidin-3-yl]carbamate (450 mg, 57%) as a white solid. MS ESI calculated for C₁₉H₂₉IN₄O₃[M+H]⁺, 489.13; found 488.95.

Preparation 107B: tert-butyl N-[(3R)-1-(4-[2-methyl-5-[(3S)-3-(2,2,2-trifluoroethyl)pyrrolidine-1-carbonylamino]phenyl]-6-(morpholin-4-yl)pyridin-2-yl)piperidin-3-yl]carbamate

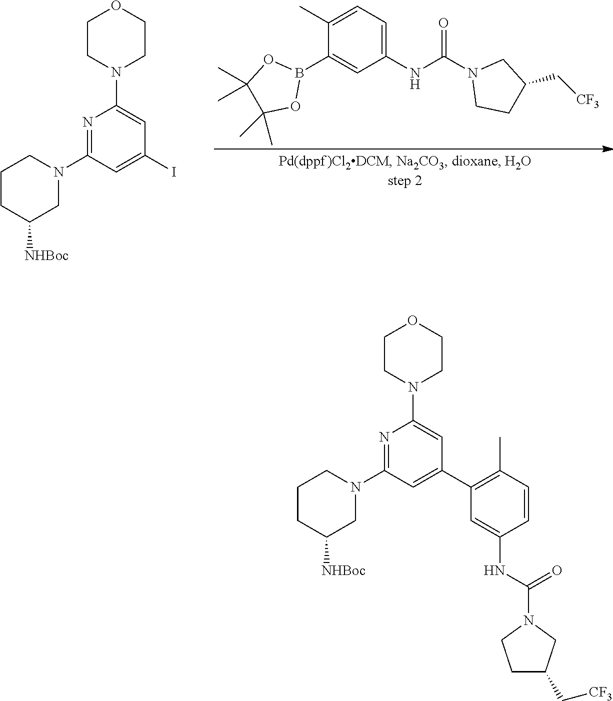 [SMILES: CC(C)(C)OC(=O)N[C@@H]1CCCN(c2cc(I)cc(N3CCOCC3)n2)C1 ; Cc1ccc(NC(=O)N2CC[C@@H](CC(F)(F)F)C2)cc1B1OC(C)(C)C(C)(C)O1 ; Cc1ccc(NC(=O)N2CC[C@@H](CC(F)(F)F)C2)cc1-c1cc(N2CCOCC2)nc(N2CCC[C@@H](NC(=O)OC(C)(C)C)C2)c1]

A mixture of tert-butyl N-[(3R)-1-[4-iodo-6-(morpholin-4-yl)pyridin-2-yl]piperidin-3-yl]carbamate (200 mg, 0.410 mmol), (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (169 mg, 0.410 mmol), 1,4-dioxane (2 mL), H₂O (0.4 mL), Na₂CO₃ (130 mg, 1.229 mmol) and Pd(dppf)Cl₂·CH₂Cl₂ (39 mg, 0.041 mmol) was stirred for 16 h at 80° C. under nitrogen atmosphere. The reaction was quenched by the addition of water (25 mL) at room temperature. The resulting mixture was extracted with EtOAc (3×25 mL). The combined organic layers were washed with brine (15 mL), dried over anhydrous Na₂SO₄. After filtration, the filtrate was concentrated under reduced pressure. The residue was purified by silica gel column chromatography, eluted with PE/EtOAc (1/1) to afford tert-butyl N-[(3R)-1-(4-[2-methyl-5-[(3S)-3-(2,2,2-trifluoroethyl)pyrrolidine-1-carbonylamino]phenyl]-6-(morpholin-4-yl)pyridin-2-yl)piperidin-3-yl]carbamate (200 mg, 76%) as a light brown solid. MS ESI calculated for C₃₃H₄₅F₃N₆O₄[M+H]⁺, 647.35, found 647.40.

### Example 107: (3S)-N-(3-[2-[(3R)-3-aminopiperidin-1-yl]-6-(morpholin-4-yl)pyridin-4-yl]-4-methylphenyl)-3-(2,2,2-trifluoroethyl)pyrrolidine-1-carboxamide

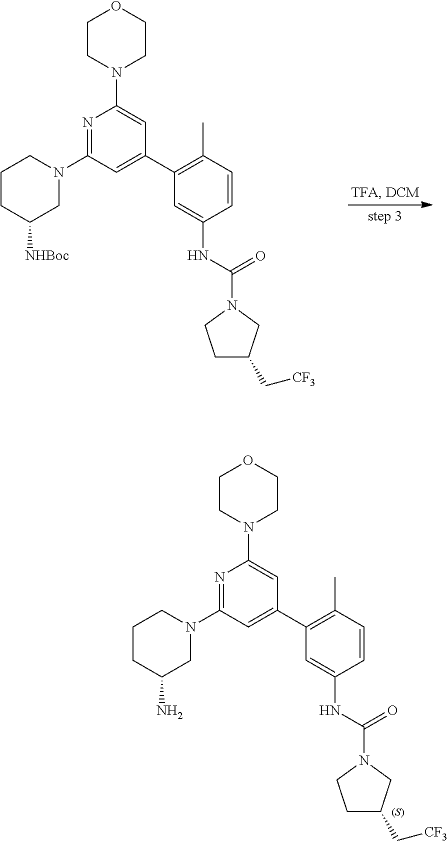

To a stirred solution of tert-butyl N-[(3R)-1-(4-[2-methyl-5-[(3S)-3-(2,2,2-trifluoroethyl)pyrrolidine-1-carbonylamino]phenyl]-6-(morpholin-4-yl)pyridin-2-yl)piperidin-3-yl]carbamate (200 mg, 0.309 mmol) in DCM (3 mL) was added TFA (1 mL) at room temperature. The resulting mixture was stirred for 1 h at room temperature. The resulting mixture was concentrated under reduced pressure. The mixture was basified to pH 8 with saturated NaHCO₃. The resulting mixture was extracted with DCM (3×20 mL). The combined organic layers were washed with brine (2×10 mL), dried over anhydrous Na₂SO₄. After filtration, the filtrate was concentrated under reduced pressure. The residue was purified by silica gel column chromatography, eluted with CH₂Cl₂/MeOH (10/1) to afford (3S)-N-(3-[2-[(3R)-3-aminopiperidin-1-yl]-6-(morpholin-4-yl)pyridin-4-yl]-4-methylphenyl)-3-(2,2,2-trifluoroethyl)pyrrolidine-1-carboxamide (94 mg, 55%) as a light yellow solid. MS ESI calculated for C₂₈H₃₇F₃N₆O₂ [M+H]⁺, 547.29, found 547.15. ¹H NMR (400 MHz, DMSO-d₆) δ 8.14 (s, 1H), 7.43 (dd, J=8.4, 2.4 Hz, 1H), 7.35 (d, J=2.4 Hz, 1H), 7.11 (d, J=8.4 Hz, 1H), 6.00 (s, 1H), 5.92 (s, 1H), 4.15-4.12 (m, 1H), 4.07-4.03 (m, 1H), 3.72-3.63 (m, 4H), 3.53-3.50 (m, 2H), 3.44-3.37 (m, 4H), 3.31-3.30 (m, 1H), 3.02-3.00 (m, 1H), 2.85-2.75 (m, 2H), 2.79-2.71 (m, 1H), 2.51-2.34 (m, 3H), 2.16 (s, 3H), 2.14-2.03 (m, 1H), 1.88-1.86 (m, 1H), 1.74-1.58 (m, 2H), 1.53-1.37 (m, 1H), 1.37-1.24 (m, 3H). ¹⁹F NMR (282 MHz, Chloroform-d) δ-64.92 (3F).

### Example 108: (3S)-N-(3-[2-[(3S)-3-aminopiperidin-1-yl]-6-(morpholin-4-yl)pyridin-4-yl]-4-methylphenyl)-3-(2,2,2-trifluoroethyl)pyrrolidine-1-carboxamide

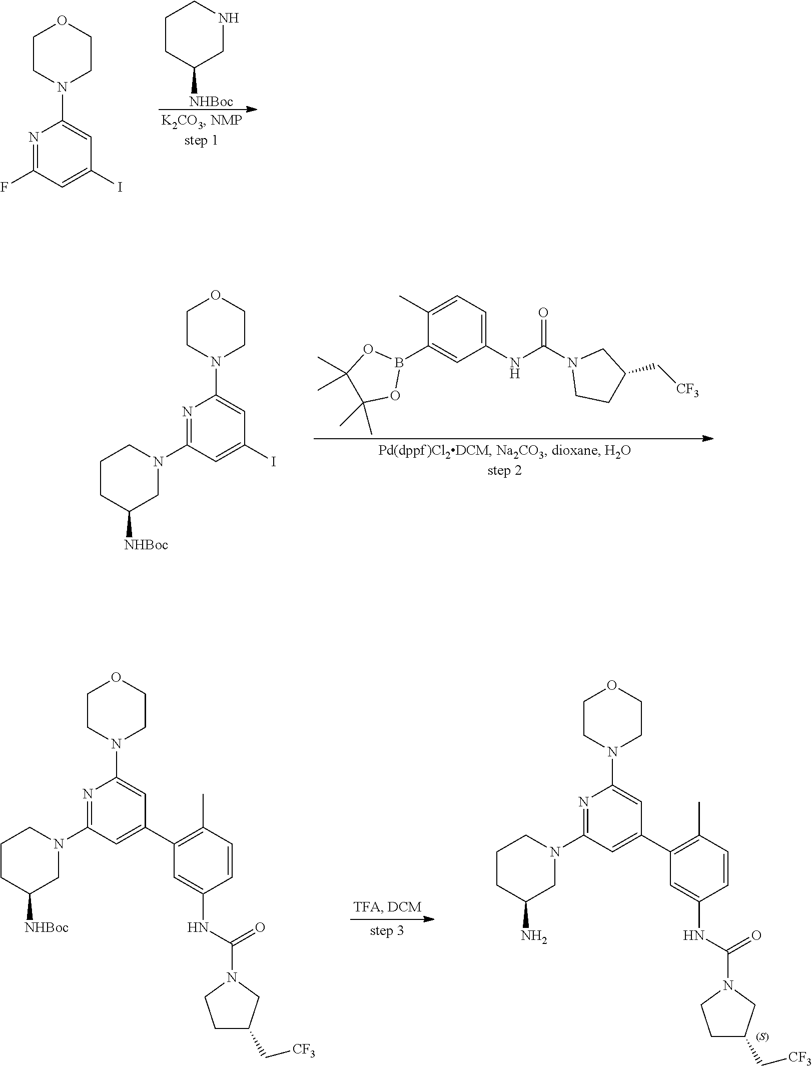

Preparation 108A: tert-butyl N-[(3S)-1-[4-iodo-6-(morpholin-4-yl)pyridin-2-yl]piperidin-3-yl]carbamate

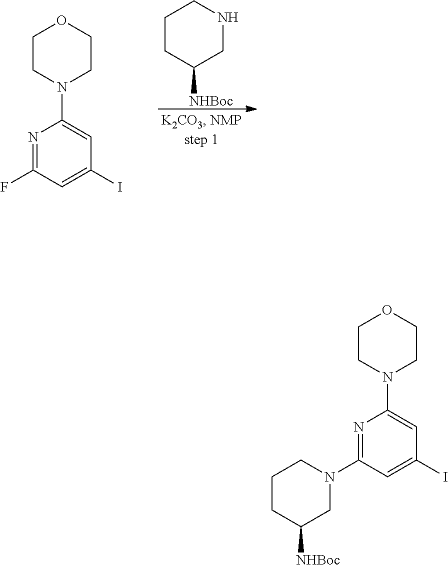 [SMILES: Fc1cc(I)cc(N2CCOCC2)n1 ; CC(C)(C)OC(=O)N[C@H]1CCCNC1 ; CC(C)(C)OC(=O)N[C@H]1CCCN(c2cc(I)cc(N3CCOCC3)n2)C1]

A mixture of 4-(6-fluoro-4-iodopyridin-2-yl)morpholine (500 mg, 1.623 mmol), tert-butyl N-[(3S)-piperidin-3-yl]carbamate(486 mg, 2.435 mmol) in NMP (5 mL) and K₂CO₃ (449 mg, 3.246 mmol) was stirred for 2 h at 150° C. The reaction was quenched by the addition of water (30 mL). The resulting mixture was extracted with EtOAc (3×40 mL). The combined organic layers were washed with brine (30 mL), dried over anhydrous Na₂SO₄. After filtration, the filtrate was concentrated under reduced pressure.The residue was purified by silica gel column chromatography, eluted with PE/EtOAc (3/1) to afford tert-butyl N-[(3S)-1-[4-iodo-6-(morpholin-4-yl)pyridin-2-yl]piperidin-3-yl]carbamate (480 mg, 61%) as a white solid. MS ESI calculated for C₁₉H₂₉IN₄O₃[M+H]⁺, 489.13; found 488.95. ¹H NMR (300 MHz, Chloroform-d) δ 6.42 (s, 1H), 6.31 (s, 1H), 4.86-4.83 (m, 1H), 3.80-3.75 (m, 4H), 3.62-3.56 (m, 2H), 3.46-3.42 (m, 6H), 1.85-1.63 (m, 4H), 1.46 (s, 9H).

Preparation 108B: tert-butyl N-[(3S)-1-(4-[2-methyl-5-[(3S)-3-(2,2,2-trifluoroethyl)pyrrolidine-1-carbonylamino]phenyl]-6-(morpholin-4-yl)pyridin-2-yl)piperidin-3-yl]carbamate

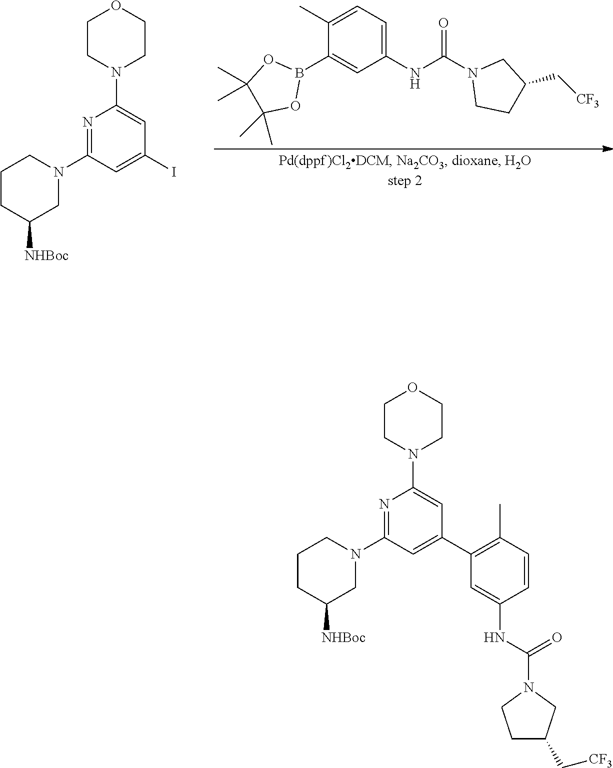 [SMILES: CC(C)(C)OC(=O)N[C@H]1CCCN(c2cc(I)cc(N3CCOCC3)n2)C1 ; Cc1ccc(NC(=O)N2CC[C@@H](CC(F)(F)F)C2)cc1B1OC(C)(C)C(C)(C)O1 ; Cc1ccc(NC(=O)N2CC[C@@H](CC(F)(F)F)C2)cc1-c1cc(N2CCOCC2)nc(N2CCC[C@H](NC(=O)OC(C)(C)C)C2)c1]

A mixture of tert-butyl N-[(3S)-1-[4-iodo-6-(morpholin-4-yl)pyridin-2-yl]piperidin-3-yl]carbamate (200 mg, 0.410 mmol), (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (169 mg, 0.410 mmol), 1,4-dioxane (2 mL), H₂O (0.4 mL), Na₂CO₃ (130 mg, 1.229 mmol) and Pd(dppf)Cl₂·CH₂Cl₂ (39 mg, 0.041 mmol) was stirred for 16 h at 80° C. under nitrogen atmosphere. The reaction was quenched by the addition of water (25 mL). The resulting mixture was extracted with EtOAc (3×25 mL). The combined organic layers were washed with brine (15 mL), dried over anhydrous Na₂SO₄. After filtration, the filtrate was concentrated under reduced pressure. The residue was purified by silica gel column chromatography, eluted with PE/EtOAc/EtOH (8/3/1) to afford tert-butyl N-[(3S)-1-(4-[2-methyl-5-[(3S)-3-(2,2,2-trifluoroethyl)pyrrolidine-1-carbonylamino]phenyl]-6-(morpholin-4-yl)pyridin-2-yl)piperidin-3-yl]carbamate (210 mg, 79%) as a light brown solid. MS ESI calculated for C₃₃H₄₅F₃N₆O₄[M+H]⁺, 647.35, found 647.45. ¹H NMR (400 MHz, Chloroform-d) δ 7.43-7.40 (m, 1H), 7.21-7.14 (m, 2H), 6.12 (s, 1H), 6.02 (s, 1H), 5.92 (s, 1H), 4.99-4.94 (m, 1H), 3.85-3.77 (m, 6H), 3.64-3.62 (m, 2H), 3.56-3.39 (m, 8H), 3.15-3.10 (m, 1H), 2.56-2.53 (m, 1H), 2.37-2.22 (m, 5H), 1.85-1.58 (m, 5H), 1.45 (s, 9H).

### Example 108: (3S)-N-(3-[2-[(3S)-3-aminopiperidin-1-yl]-6-(morpholin-4-yl)pyridin-4-yl]-4-methylphenyl)-3-(2,2,2-trifluoroethyl)pyrrolidine-1-carboxamide

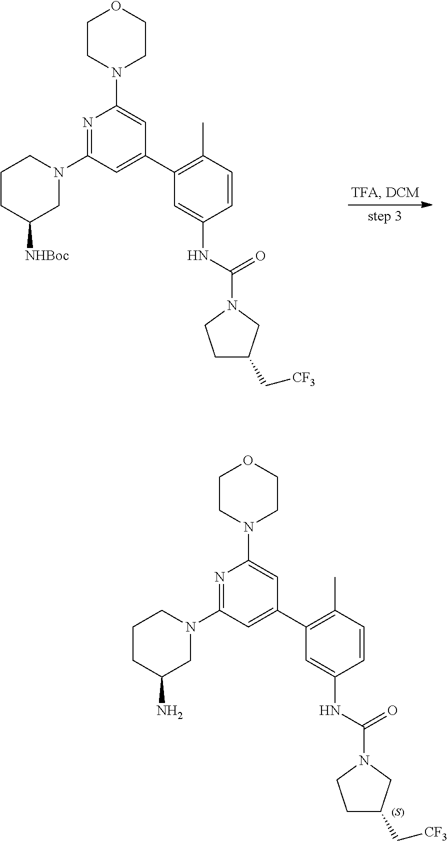

To a stirred solution of tert-butyl N-[(3S)-1-(4-[2-methyl-5-[(3S)-3-(2,2,2-trifluoroethyl)pyrrolidine-1-carbonylamino]phenyl]-6-(morpholin-4-yl)pyridin-2-yl)piperidin-3-yl]carbamate (200 mg, 0.309 mmol) in DCM (3 mL) was added TFA (1 mL). The resulting mixture was stirred for 1 h at room temperature. The resulting mixture was concentrated under reduced pressure. The mixture was basified to pH 8 with saturated NaHCO₃. The resulting mixture was extracted with DCM (3×20 mL). The combined organic layers were washed with brine (2×10 mL), dried over anhydrous Na₂SO₄. After filtration, the filtrate was concentrated under reduced pressure. The residue was purified by silica gel column chromatography, eluted with CH₂Cl₂/MeOH (10/1) to afford (3S)-N-(3-[2-[(3S)-3-aminopiperidin-1-yl]-6-(morpholin-4-yl)pyridin-4-yl]-4-methylphenyl)-3-(2,2,2-trifluoroethyl)pyrrolidine-1-carboxamide (107 mg, 63%) as a light yellow solid. MS ESI calculated for C₂₈H₃₇F₃N₆O₂ [M+H]⁺, 547.29, found 547.15. ¹H NMR (400 MHz, DMSO-d₆) δ 8.14 (s, 1H), 7.43 (dd, J=8.4, 2.0 Hz, 1H), 7.35 (d, J=2.0 Hz, 1H), 7.11 (d, J=8.4 Hz, 1H), 5.99 (s, 1H), 5.91 (s, 1H), 4.15-4.05 (m, 2H), 3.72-3.63 (m, 5H), 3.53-3.50 (m, 1H), 3.44-3.37 (m, 4H), 3.31-3.29 (m, 1H), 3.02-3.00 (m, 1H), 2.85-2.75 (m, 2H), 2.79-2.71 (m, 1H), 2.51-2.34 (m, 3H), 2.16 (s, 3H), 2.14-2.03 (m, 1H), 1.88-1.86 (m, 1H), 1.74-1.58 (m, 2H), 1.53-1.37 (m, 1H), 1.37-1.24 (m, 2H). ¹⁹F NMR (282 MHz, Chloroform-d) δ-64.93 (3F).

### Example 109: (3S)-N-[4-methyl-3-[2-(morpholin-4-yl)-6-(2-oxopyrrolidin-1-yl)pyridin-4-yl]phenyl]-3-(2,2,2-trifluoroethyl)pyrrolidine-1-carboxamide

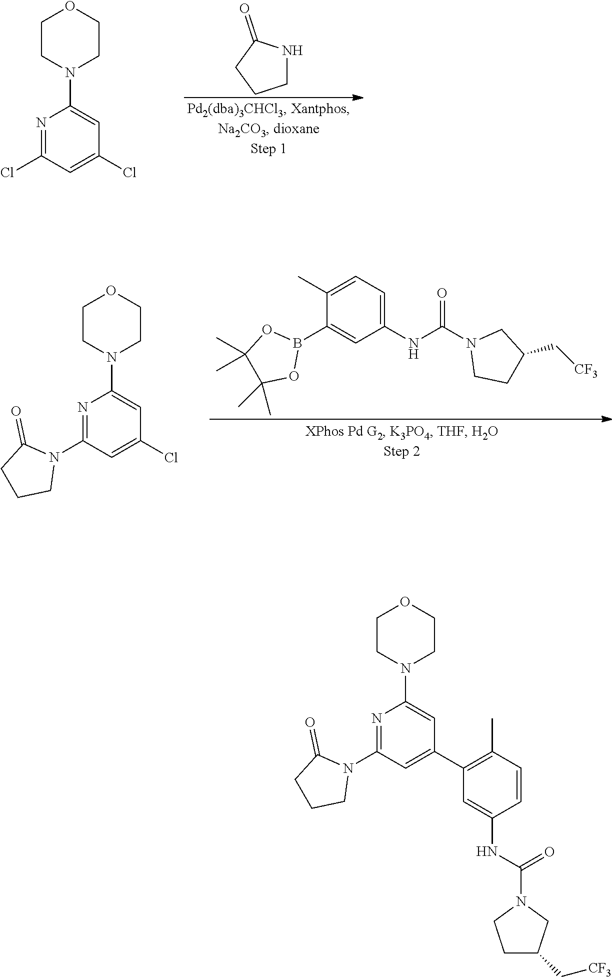 [SMILES: Clc1cc(Cl)nc(N2CCOCC2)c1 ; O=C1CCCN1 ; O=C1CCCN1c1cc(Cl)cc(N2CCOCC2)n1 ; Cc1ccc(NC(=O)N2CC[C@@H](CC(F)(F)F)C2)cc1B1OC(C)(C)C(C)(C)O1 ; Cc1ccc(NC(=O)N2CC[C@@H](CC(F)(F)F)C2)cc1-c1cc(N2CCOCC2)nc(N2CCCC2=O)c1]

Preparation 109A: 1-[4-chloro-6-(morpholin-4-yl)pyridin-2-yl]pyrrolidin-2-one

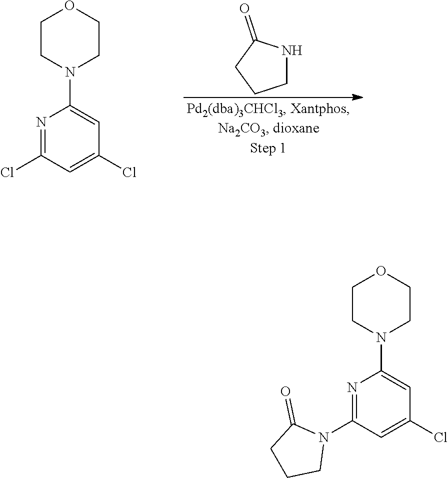 [SMILES: Clc1cc(Cl)nc(N2CCOCC2)c1 ; O=C1CCCN1 ; O=C1CCCN1c1cc(Cl)cc(N2CCOCC2)n1]

A mixture of 4-(4,6-dichloropyridin-2-yl)morpholine (1.00 g, 4.29 mmol) and pyrrolidone (0.55 g, 6.435 mmol), 1,4-dioxane (20 mL), Na₂CO₃ (0.91 g, 8.58 mmol), XantPhos (0.50 g, 0.86 mmol) and Pd₂(dba)₃·CHCl₃ (0.44 g, 0.43 mmol) was stirred for 16 h at 100° C. under nitrogen atmosphere. The reaction was quenched by the addition of water (100 mL). The resulting mixture was extracted with EtOAc (3×100 mL). The combined organic layers were washed with brine (2×100 mL), dried over anhydrous Na₂SO₄. After filtration, the filtrate was concentrated under reduced pressure. The residue was purified by silica gel column chromatography, eluted with PE/EtOAc/EtOH (9/3/1) to afford 1-[4-chloro-6-(morpholin-4-yl)pyridin-2-yl]pyrrolidin-2-one (690 mg, 57%) as a light yellow solid. MS ESI calculated for C₁₃H₁₆ClN₃O₂[M+H]⁺, 282.09, found 282.15. ¹H NMR (400 MHz, Chloroform-d) δ 7.86 (d, J=1.2 Hz, 1H), 6.38 (d, J=1.2 Hz, 1H), 4.10-4.02 (m, 2H), 3.86-3.79 (m, 4H), 3.52-3.45 (m, 4H), 2.66-2.64 (m, 2H), 2.17-2.05 (m, 2H).

### Example 109: (3S)-N-[4-methyl-3-[2-(morpholin-4-yl)-6-(2-oxopyrrolidin-1-yl)pyridin-4-yl]phenyl]-3-(2,2,2-trifluoroethyl)pyrrolidine-1-carboxamide

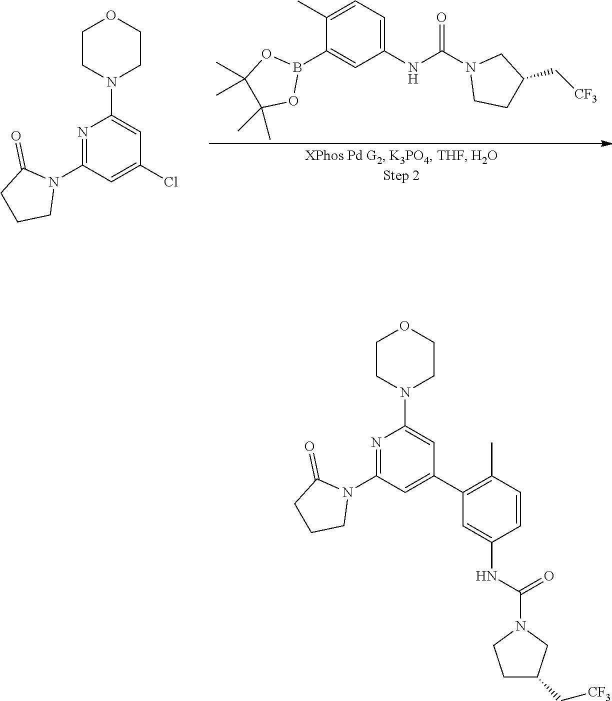 [SMILES: O=C1CCCN1c1cc(Cl)cc(N2CCOCC2)n1 ; Cc1ccc(NC(=O)N2CC[C@@H](CC(F)(F)F)C2)cc1B1OC(C)(C)C(C)(C)O1 ; Cc1ccc(NC(=O)N2CC[C@@H](CC(F)(F)F)C2)cc1-c1cc(N2CCOCC2)nc(N2CCCC2=O)c1]

A mixture of 1-[4-chloro-6-(morpholin-4-yl)pyridin-2-yl]pyrrolidin-2-one (150 mg, 0.532 mmol,), (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (219 mg, 0.532 mmol), 1,4-dioxane (2 mL), H₂O (0.4 mL), K₃PO₄ (38 mg, 0.355 mmol) and 2^{nd} XPhos Precatalyst (14 mg, 0.018 mmol) was stirred for 16 h at 80° C. under nitrogen atmosphere. The mixture was allowed to cool down to room temperature. The reaction was quenched by the addition of water (10 mL). The resulting mixture was extracted with EtOAc (3×20 mL). The combined organic layers were washed with brine (20 mL), dried over anhydrous Na₂SO₄. After filtration, the filtrate was concentrated under reduced pressure. The residue was purified by silica gel column chromatography, eluted with PE/EtOAc/EtOH (4/3/1) to afford (3S)-N-[4-methyl-3-[2-(morpholin-4-yl)-6-(2-oxopyrrolidin-1-yl)pyridin-4-yl]phenyl]-3-(2,2,2-trifluoroethyl)pyrrolidine-1-carboxamide (117.7 mg, 42%) as a white solid. MS ESI calculated for C₂₇H₃₂F₃N₅O₃ [M+H]⁺, 532.25, found 532.20. ¹H NMR (400 MHz, DMSO-d₆) δ 8.17 (s, 1H), 7.58 (s, 1H), 7.47 (dd, J=8.4, 2.4 Hz, 1H), 7.38 (d, J=2.4 Hz, 1H), 7.14 (d, J=8.4 Hz, 1H), 6.44 (s, 1H), 4.02 (t, J=7.2 Hz, 2H), 3.74-3.63 (m, 5H), 3.58-3.43 (m, 5H), 3.34-3.32 (m, 1H), 3.04-3.02 (m, 1H), 2.61-2.51 (m, 2H), 2.50-2.30 (m, 3H), 2.16 (s, 3H), 2.13-1.95 (m, 3H), 1.66-1.64 (m, 1H). ¹⁹F NMR (376 MHz, DMSO-d₆) δ-63.37 (3F).

### Example 110: (3S)-N-(3-[2-[(3R)-3-hydroxypyrrolidin-1-yl]-6-(morpholin-4-yl)pyridin-4-yl]-4-methylphenyl)-3-(2,2,2-trifluoroethyl)pyrrolidine-1-carboxamide

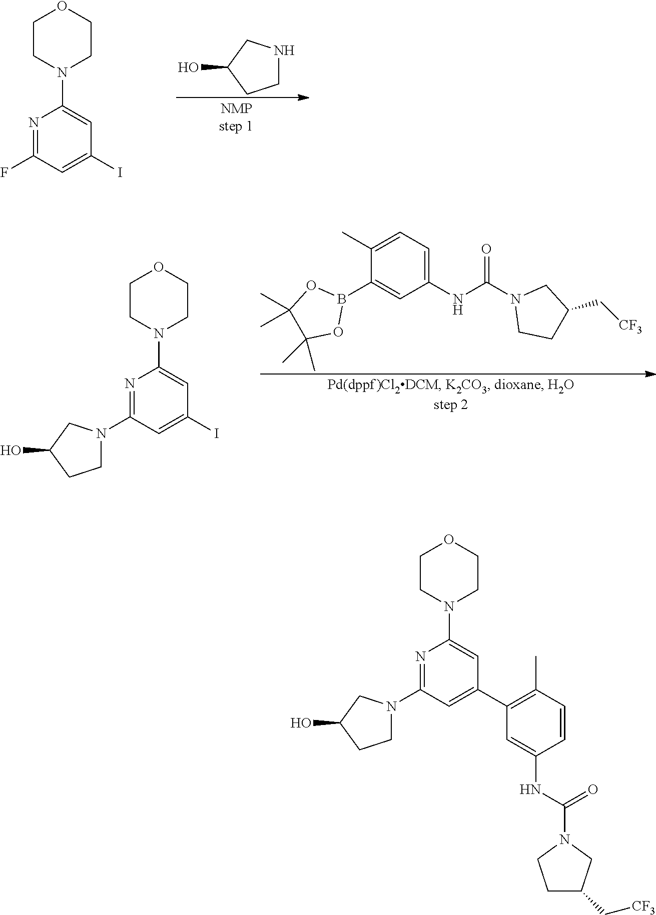 [SMILES: Fc1cc(I)cc(N2CCOCC2)n1 ; O[C@@H]1CCNC1 ; O[C@@H]1CCN(c2cc(I)cc(N3CCOCC3)n2)C1 ; Cc1ccc(NC(=O)N2CC[C@@H](CC(F)(F)F)C2)cc1B1OC(C)(C)C(C)(C)O1 ; Cc1ccc(NC(=O)N2CC[C@@H](CC(F)(F)F)C2)cc1-c1cc(N2CCOCC2)nc(N2CC[C@@H](O)C2)c1]

Preparation 110A: (3R)-1-[4-iodo-6-(morpholin-4-yl)pyridin-2-yl]pyrrolidin-3-ol

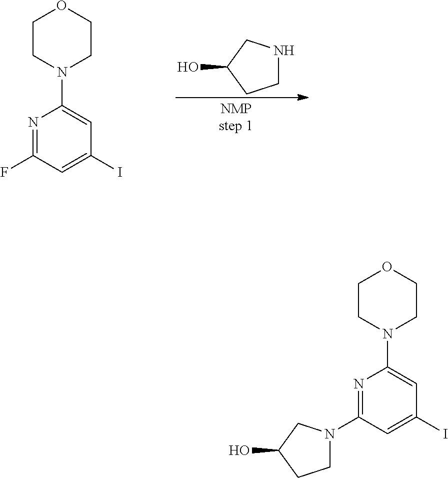 [SMILES: Fc1cc(I)cc(N2CCOCC2)n1 ; O[C@@H]1CCNC1 ; O[C@@H]1CCN(c2cc(I)cc(N3CCOCC3)n2)C1]

A mixture of 4-(6-fluoro-4-iodopyridin-2-yl)morpholine (500 mg, 1.623 mmol) and (3R)-pyrrolidin-3-ol (212 mg, 2.434 mmol) in NMP (6 mL) and K₂CO₃ (448 mg, 3.246 mmol) was stirred for 2 h at 150° C. under nitrogen atmosphere. The reaction was quenched by the addition of water (100 mL). The resulting mixture was extracted with EtOAc (3×300 mL). The combined organic layers were washed with brine (3×200 mL), dried over anhydrous Na₂SO₄. After filtration, the filtrate was concentrated under reduced pressure. The residue was purified by silica gel column chromatography, eluted with PE/EtOAc (1/1) to afford (3R)-1-[4-iodo-6-(morpholin-4-yl)pyridin-2-yl]pyrrolidin-3-ol (572 mg, 94%) as an off-white solid. MS ESI calculated for C₁₃H₁₈IN₃O₂[M+H]⁺, 376.04, found 376.00. ¹H NMR (400 MHz, DMSO-d₆) δ 6.30 (s, 1H), 6.10 (s, 1H), 4.90 (d, J=3.6 Hz, 1H), 4.38-4.28 (m, 1H), 3.65-3.63 (m, 4H), 3.45-3.33 (m, 7H), 3.23-3.21 (m, 1H), 2.00-1.95 (m, 1H), 1.89-1.78 (m, 1H).

### Example 110: (3S)-N-(3-[2-[(3R)-3-hydroxypyrrolidin-1-yl]-6-(morpholin-4-yl)pyridin-4-yl]-4-methylphenyl)-3-(2,2,2-trifluoroethyl)pyrrolidine-1-carboxamide

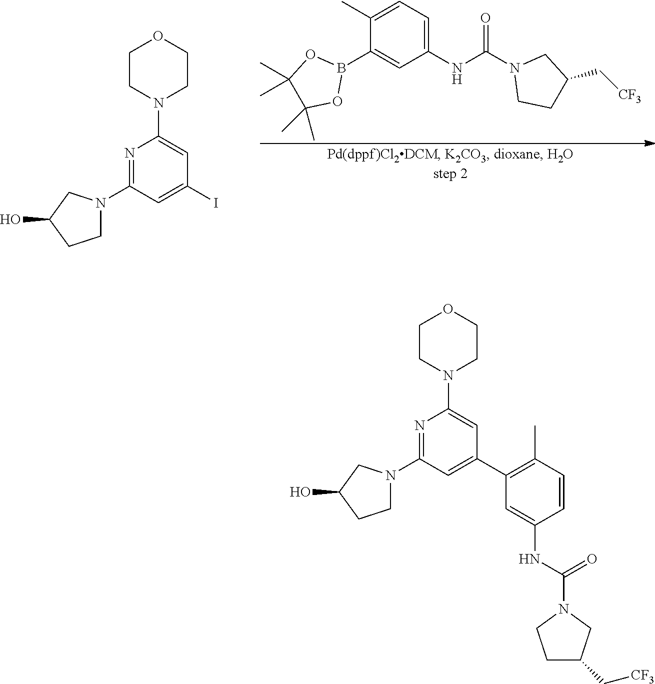 [SMILES: O[C@@H]1CCN(c2cc(I)cc(N3CCOCC3)n2)C1 ; Cc1ccc(NC(=O)N2CC[C@@H](CC(F)(F)F)C2)cc1B1OC(C)(C)C(C)(C)O1 ; Cc1ccc(NC(=O)N2CC[C@@H](CC(F)(F)F)C2)cc1-c1cc(N2CCOCC2)nc(N2CC[C@@H](O)C2)c1]

A mixture of (3R)-1-[4-iodo-6-(morpholin-4-yl)pyridin-2-yl]pyrrolidin-3-ol (120 mg, 0.320 mmol), (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (105 mg, 0.256 mmol) in dioxane (4 mL) and H₂O (1 mL), Pd(dppf)Cl₂·CH₂Cl₂ (26 mg, 0.032 mmol) and Na₂CO₃ (102 mg, 0.959 mmol) was stirred for 2 h at 60° C. under nitrogen atmosphere. The reaction mixture was concentrated under reduced pressure. The residue was purified by Prep-TLC with PE/EtOAc (1/1). The crude product was purified by Prep-HPLC with the following conditions (Column: XBridge Prep Phenyl OBD Column, 19×150 mm, 5 μm, 13 nm; Mobile Phase A: water (10 mmoL/L NH₄HCO₃), Mobile Phase B: CH₃CN; Flow rate:20 mL/min; Gradient: 40% B to 60% B in 4.3 min; 254/210 nm to afford (3S)-N-(3-[2-[(3R)-3-hydroxypyrrolidin-1-yl]-6-(morpholin-4-yl)pyridin-4-yl]-4-methylphenyl)-3-(2,2,2-trifluoroethyl)pyrrolidine-1-carboxamide (53.7 mg, 31%) as an light blue solid. MS ESI calculated for C₂₇H₃₄F₃N₅O₃[M+H]⁺, 534.26, found 534.20. ¹H NMR (400 MHz, DMSO-d₆) δ 8.12 (s, 1H), 7.43 (dd, J=8.4, 2.4 Hz, 1H), 7.34 (d, J=2.4 Hz, 1H), 7.10 (d, J=8.4 Hz, 1H), 5.84 (s, 1H), 5.64 (s, 1H), 4.90 (d, J=3.6 Hz, 1H), 4.36-4.34 (m, 1H), 3.72-3.63 (m, 5H), 3.53-3.42 (m, 8H), 3.33-3.30 (m, 2H), 3.04-3.00 (m, 1H), 2.50-2.34 (m, 3H), 2.17 (s, 3H), 2.10-1.94 (m 2H), 1.88-1.82 (m, 1H), 1.73-1.58 (m, 1H). ¹⁹F NMR (376 MHz, DMSO-d₆) δ-63.37 (3F).

### Example 111: (3S)-N-[4-methyl-3-[2-(3-methyl-2-oxoimidazolidin-1-yl)-6-(morpholin-4-yl)pyridin-4-yl]phenyl]-3-(2,2,2-trifluoroethyl)pyrrolidine-1-carboxamide

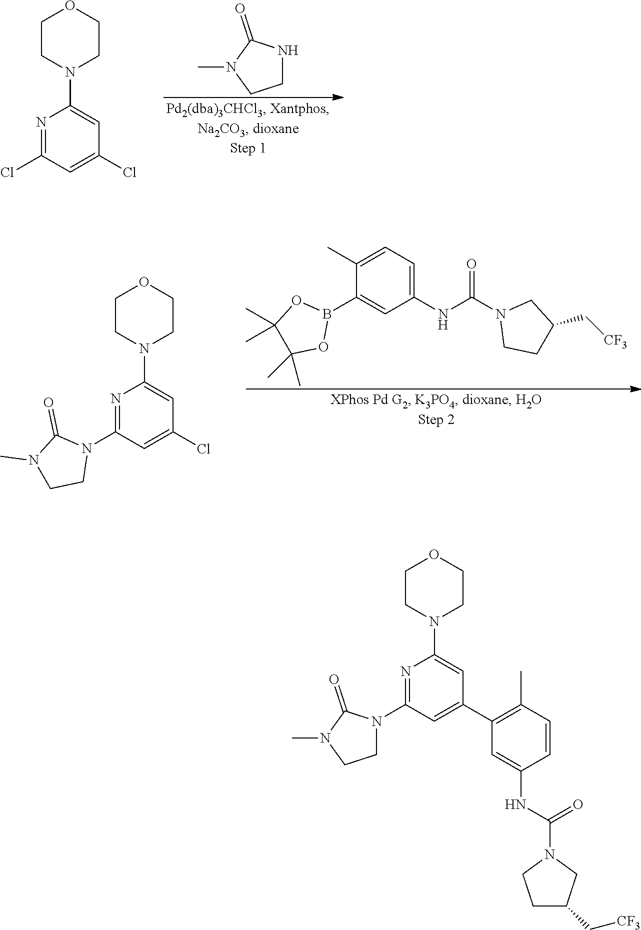

Preparation 111A: 1-[4-chloro-6-(morpholin-4-yl)pyridin-2-yl]-3-methylimidazolidin-2-one

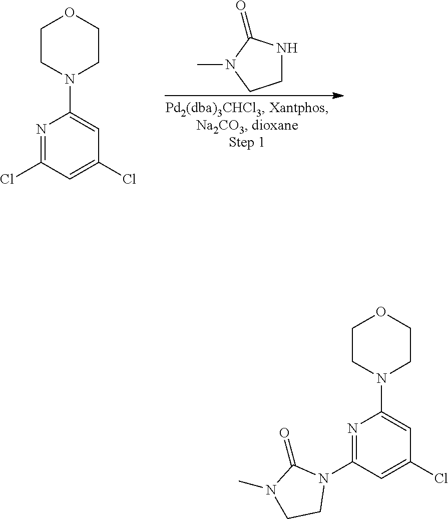

To a stirred solution of 4-(4,6-dichloropyridin-2-yl)morpholine (500 mg, 2.145 mmol) and 1-methylimidazolidin-2-one (322 mg, 3.218 mmol) in 1,4-dioxane (5 mL) were added Na₂CO₃ (682 mg, 6.435 mmol), XantPhos (248 mg, 0.429 mmol) and Pd₂(dba)₃CHCl₃ (222 mg, 0.215 mmol) under nitrogen atmosphere. The resulting mixture was stirred for 16 h at 100° C. under nitrogen atmosphere. The reaction was quenched by the addition of water (20 mL). The resulting mixture was extracted with EtOAc (3×20 mL). The combined organic layers were washed with brine (20 mL), dried over anhydrous Na₂SO₄. After filtration, the filtrate was concentrated under reduced pressure. The residue was purified by silica gel column chromatography, eluted with PE/EtOAc (1/1) to afford 1-[4-chloro-6-(morpholin-4-yl)pyridin-2-yl]-3-methylimidazolidin-2-one (500 mg, 79%) as a light yellow solid. MS ESI calculated for C₁₃H₁₇ClN₄O₂[M+H]⁺, 297.10; found 297.15. ¹H NMR (400 MHz, Chloroform-d) δ 7.74 (d, J=1.2 Hz, 1H), 6.23 (d, J=1.2 Hz, 1H), 4.03-3.99 (m, 2H), 3.83-3.80 (m, 4H), 3.48-3.43 (m, 6H), 2.92 (s, 3H).

### Example 111: (3S)-N-[4-methyl-3-[2-(3-methyl-2-oxoimidazolidin-1-yl)-6-(morpholin-4-yl)pyridin-4-yl]phenyl]-3-(2,2,2-trifluoroethyl)pyrrolidine-1-carboxamide

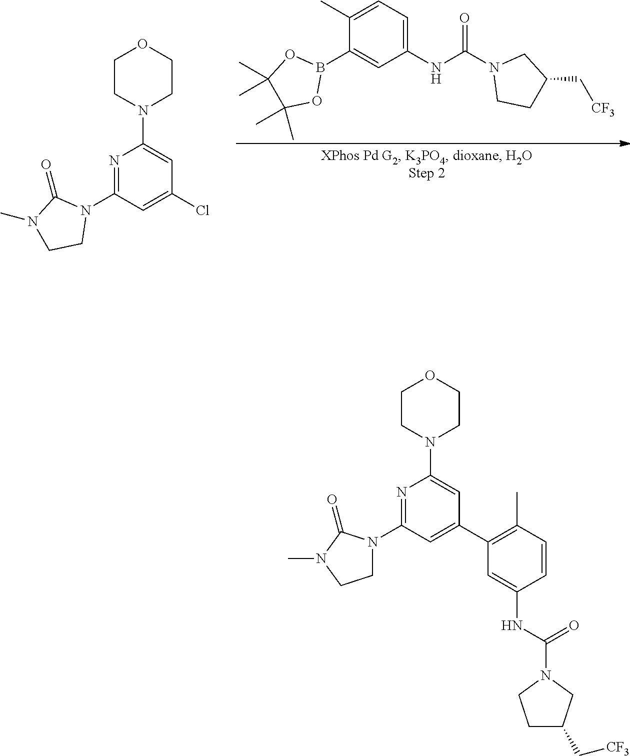 [SMILES: CN1CCN(c2cc(Cl)cc(N3CCOCC3)n2)C1=O ; Cc1ccc(NC(=O)N2CC[C@@H](CC(F)(F)F)C2)cc1B1OC(C)(C)C(C)(C)O1 ; Cc1ccc(NC(=O)N2CC[C@@H](CC(F)(F)F)C2)cc1-c1cc(N2CCOCC2)nc(N2CCN(C)C2=O)c1]

To a stirred solution of 1-[4-chloro-6-(morpholin-4-yl)pyridin-2-yl]pyrrolidin-2-one (150 mg, 0.532 mmol,) and (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (219 mg, 0.532 mmol) in 1,4-dioxane (2 mL) and H₂O (0.4 mL) were added K₃PO₄ (38 mg, 0.355 mmol) and 2^{nd} XPhos Precatalyst (14 mg, 0.018 mmol) under nitrogen atmosphere. The resulting mixture was stirred for 16 h at 80° C. under nitrogen atmosphere. The reaction was quenched by the addition of water (10 mL). The resulting mixture was extracted with EtOAc (3×20 mL). The combined organic layers were washed with brine (20 mL), dried over anhydrous Na₂SO₄. After filtration, the filtrate was concentrated under reduced pressure. The residue was purified by silica gel column chromatography, eluted with PE/EtOAc/EtOH (4/3/1) to afford (3S)-N-[4-methyl-3-[2-(morpholin-4-yl)-6-(2-oxopyrrolidin-1-yl)pyridin-4-yl]phenyl]-3-(2,2,2-trifluoroethyl)pyrrolidine-1-carboxamide (113.5 mg, 41%) as a white solid. MS ESI calculated for C₂₇H₃₃F₃N₆O₃ [M+H]⁺, 547.26, found 547.15. ¹H NMR (400 MHz, DMSO-d₆) δ 8.16 (s, 1H), 7.47-7.44 (m, 2H), 7.37 (s, 1H), 7.13 (d, J=8.8 Hz, 1H), 6.28 (s, 1H), 3.99-3.91 (m, 2H), 3.72-3.63 (m, 5H), 3.56-3.38 (m, 7H), 3.34-3.25 (m, 1H), 3.03 (t, J=9.6 Hz, 1H), 2.77 (s, 3H), 2.51-2.34 (m, 3H), 2.16 (s, 3H), 2.11-2.07 (m, 1H), 1.70-1.60 (m, 1H). ¹⁹F NMR (282 MHz, DMSO-d₆) δ-69.69 (3F).

### Example 112: (3S)-N-[4-methyl-3-[2-(N-methylacetamido)-6-(morpholin-4-yl)pyridin-4-yl]phenyl]-3-(2,2,2-trifluoroethyl)pyrrolidine-1-carboxamide

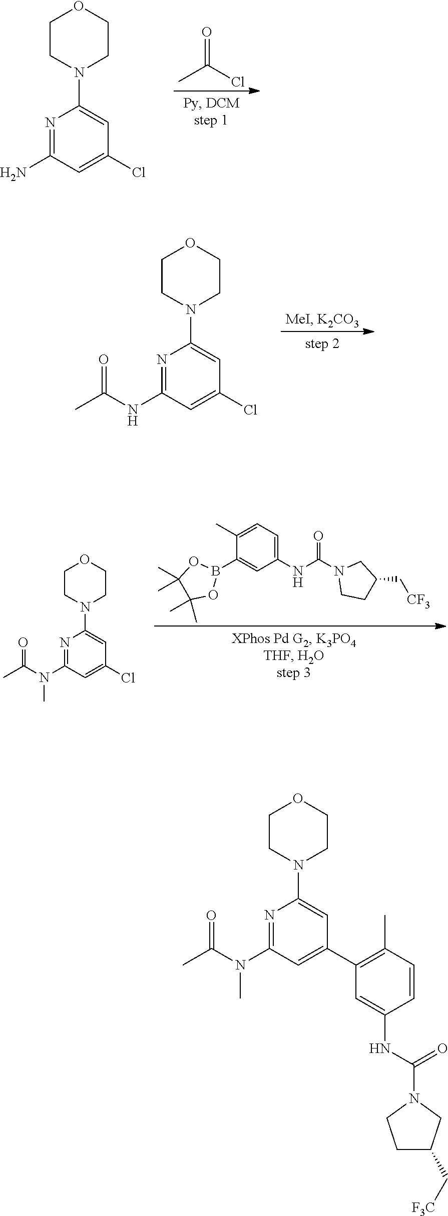

Preparation 112A: tert-butyl N-[4-chloro-6-(morpholin-4-yl)pyridin-2-yl]acetamide

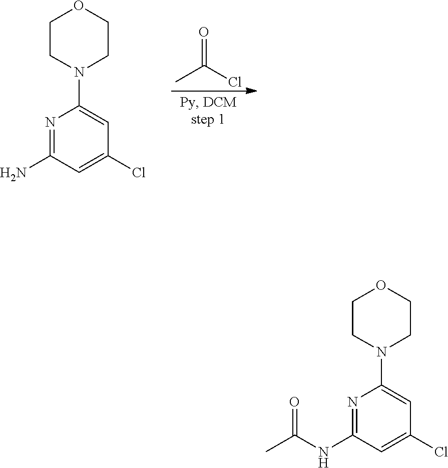

To a stirred mixture of 4-chloro-6-(morpholin-4-yl)pyridin-2-amine (300 mg, 1.404 mmol) and Pyridine (222 mg, 2.808 mmol) in DCM (5 mL) was added acetyl chloride (220 mg, 2.808 mmol) at 0° C. The resulting mixture was stirred for 2 h at room temperature. The reaction was quenched by the addition of water (10 mL). The resulting mixture was extracted with EtOAc (3×10 mL). The combined organic layers were washed with brine (2×5 mL), dried over anhydrous Na₂SO₄. After filtration, the filtrate was concentrated under reduced pressure. The residue was purified by silica gel column chromatography, eluted with PE/EtOAc (1/1) to afford N-[4-chloro-6-(morpholin-4-yl)pyridin-2-yl]acetamide (224 mg, 62%) as an off-white solid. MS ESI calculated for Cn H₁₄ClN₃O₂[M+H]⁺, 256.08, found 256.10. ¹H NMR (400 MHz, DMSO-d₆) δ 7.41 (s, 1H), 6.59 (s, 1H), 3.66 (m, 4H), 3.47 (m, 4H), 2.06 (s, 3H).

Preparation 112B: N-[4-chloro-6-(morpholin-4-yl)pyridin-2-yl]-N-methylacetamide

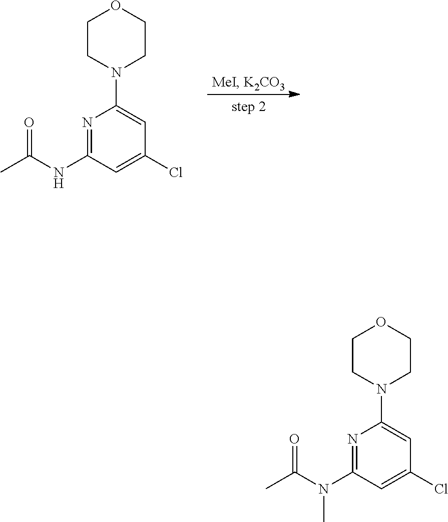 [SMILES: CC(=O)Nc1cc(Cl)cc(N2CCOCC2)n1 ; CC(=O)N(C)c1cc(Cl)cc(N2CCOCC2)n1]

To a stirred mixture of N-[4-chloro-6-(morpholin-4-yl)pyridin-2-yl]acetamide (200 mg, 0.782 mmol) and NaH (28 mg, 1.173 mmol) in DMF (8 mL) was added Mel (167 mg, 1.173 mmol) dropwise at 0° C. The resulting mixture was stirred for 5 h at room temperature under nitrogen atmosphere. The resulting mixture was diluted with water (20 mL). The resulting mixture was extracted with EtOAc (3×10 mL). The combined organic layers were washed with brine (2×10 mL), dried over anhydrous Na₂SO₄. After filtration, the filtrate was concentrated under reduced pressure. The residue was purified by silica gel column chromatography, eluted with PE/EtOAc (1/1) to afford N-[4-chloro-6-(morpholin-4-yl)pyridin-2-yl]-N-methylacetamide (166 mg, 79%) as an off-white solid. MS ESI calculated for C₁₂H₁₆ClN₃O₂[M+H]⁺, 270.09, found 270.20. ¹H NMR (300 MHz, Chloroform-d) δ 6.63 (s, 1H), 6.49 (s, 1H), 3.87-3.78 (m, 4H), 3.57-3.47 (m, 4H), 3.33 (s, 3H), 2.16 (s, 3H).

### Example 112: (3S)-N-[4-methyl-3-[2-(N-methylacetamido)-6-(morpholin-4-yl)pyridin-4-yl]phenyl]-3-(2,2,2-trifluoroethyl)pyrrolidine-1-carboxamide

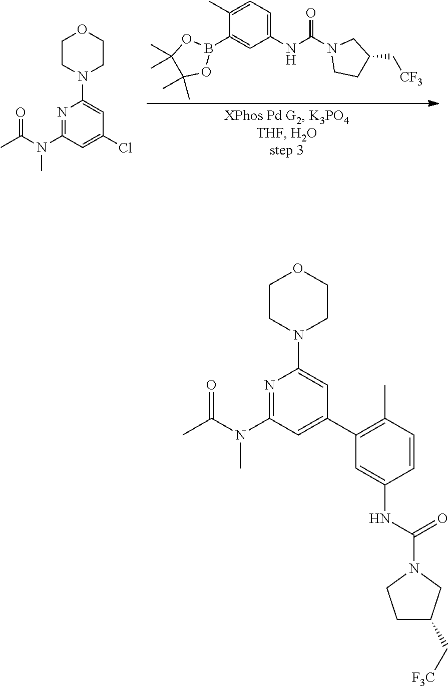 [SMILES: CC(=O)N(C)c1cc(Cl)cc(N2CCOCC2)n1 ; Cc1ccc(NC(=O)N2CC[C@@H](CC(F)(F)F)C2)cc1B1OC(C)(C)C(C)(C)O1 ; CC(=O)N(C)c1cc(-c2cc(NC(=O)N3CC[C@@H](CC(F)(F)F)C3)ccc2C)cc(N2CCOCC2)n1]

To a stirred solution of N-[4-chloro-6-(morpholin-4-yl)pyridin-2-yl]-N-methylacetamide (140 mg, 0.519 mmol) and (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (148 mg, 0.519 mmol) in THF (5 mL) and H₂O (0.5 mL) were added 2^{nd} XPhos Precatalyst (41 mg, 0.052 mmol) and K₃PO₄ (220 mg, 1.038 mmol). The resulting mixture was stirred for 1 h at 50° C. under nitrogen atmosphere. The resulting mixture was diluted with water (30 mL). The resulting mixture was extracted with EtOAc (3×10 mL). The combined organic layers were washed with brine (2×10 mL), dried over anhydrous Na₂SO₄. After filtration, the filtrate was concentrated under reduced pressure. The residue was purified by Prep-TLC with PE/EtOAc (1/1) to afford (3S)-N-[4-methyl-3-[2-(N-methylacetamido)-6-(morpholin-4-yl)pyridin-4-yl]phenyl]-3-(2,2,2-trifluoroethyl)pyrrolidine-1-carboxamide (113 mg, 41%) as an off-white solid. MS ESI calculated for C₂₆H₃₂F₃N₅O₃[M+H]⁺, 520.25, found 520.15. ¹H NMR (400 MHz, Chloroform-d) δ 7.54 (s, 1H), 7.42-7.41 (m, 1H), 7.28-7.02 (m, 1H), 6.53 (s, 1H), 6.48 (s, 1H), 6.17 (s, 1H), 3.86-3.80 (m, 5H), 3.67-3.65 (m, 1H), 3.63-3.54 (m, 4H), 3.49-3.43 (m, 1H), 3.36 (s, 3H), 3.14 (t, J=9.6 Hz, 1H), 2.63-2.54 (m, 1H), 2.35-2.28 (m, 2H), 2.24 (s, 3H), 2.16 (s, 3H), 1.90-1.77 (m, 2H). ¹⁹F NMR (376 MHz, Chloroform-d) δ-64.96 (3F).

### Example 113: (3S)-N-[3-[2-methanesulfonamido-6-(morpholin-4-yl)pyridin-4-yl]-4-methylphenyl]-3-(2,2,2-trifluoroethyl)pyrrolidine-1-carboxamide

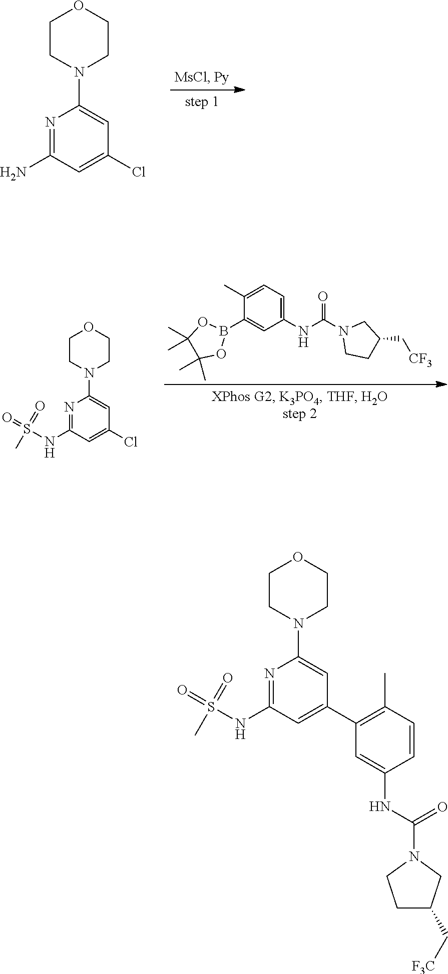 [SMILES: Nc1cc(Cl)cc(N2CCOCC2)n1 ; CS(=O)(=O)Nc1cc(Cl)cc(N2CCOCC2)n1 ; Cc1ccc(NC(=O)N2CC[C@@H](CC(F)(F)F)C2)cc1B1OC(C)(C)C(C)(C)O1 ; Cc1ccc(NC(=O)N2CC[C@@H](CC(F)(F)F)C2)cc1-c1cc(NS(C)(=O)=O)nc(N2CCOCC2)c1]

Preparation 113A: N-[4-chloro-6-(morpholin-4-yl)pyridin-2-yl]methanesulfonamide

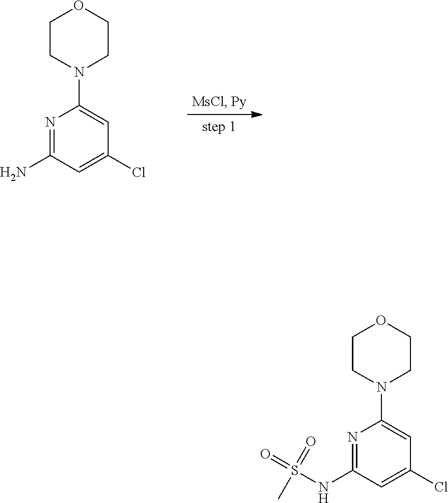 [SMILES: Nc1cc(Cl)cc(N2CCOCC2)n1 ; CS(=O)(=O)Nc1cc(Cl)cc(N2CCOCC2)n1]

A mixture of 4-chloro-6-(morpholin-4-yl)pyridin-2-amine (300 mg, 1.404 mmol), MsCl (209 mg, 1.8 mmol) in pyridine (5 mL) was stirred for 2 h at 80° C. The resulting mixture was concentrated under reduced pressure. The residue was purified by silica gel column chromatography, eluted with EtOAc/EtOH (3/1) in petroleum ether (70%) to afford N-[4-chloro-6-(morpholin-4-yl)pyridin-2-yl]methanesulfonamide (350 mg, 85%) as a light pink solid. MS ESI calculated for C₁₀H₁₄ClN₃O₃S [M+H]⁺, 292.04, found 291.95. ¹H NMR (400 MHz, Chloroform-d) δ 6.51 (d, J=1.2 Hz, 1H), 6.35 (d, J=1.2 Hz, 1H), 3.85-3.78 (m, 4H), 3.54-3.47 (m, 4H), 3.25 (s, 3H).

### Example 113: (3S)-N-[3-[2-methanesulfonamido-6-(morpholin-4-yl)pyridin-4-yl]-4-methylphenyl]-3-(2,2,2-trifluoroethyl)pyrrolidine-1-carboxamide

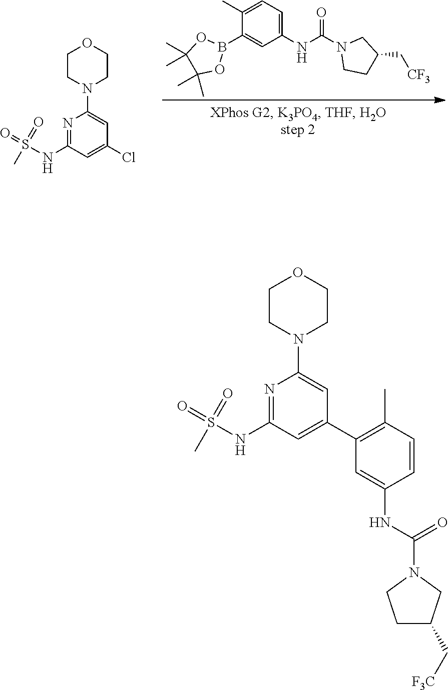 [SMILES: CS(=O)(=O)Nc1cc(Cl)cc(N2CCOCC2)n1 ; Cc1ccc(NC(=O)N2CC[C@@H](CC(F)(F)F)C2)cc1B1OC(C)(C)C(C)(C)O1 ; Cc1ccc(NC(=O)N2CC[C@@H](CC(F)(F)F)C2)cc1-c1cc(NS(C)(=O)=O)nc(N2CCOCC2)c1]

A mixture of N-[4-chloro-6-(morpholin-4-yl)pyridin-2-yl]methanesulfonamide (170 mg, 0.6 mmol), (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (192 mg, 0.5 mmol), K₃PO₄ (371 mg, 1.8 mmol) and 2^{nd} XPhos Precatalyst (46 mg, 0.06 mmol) in and H₂O (0.4 mL), THF (4 mL) was stirred for 2 h at 80° C. under nitrogen atmosphere. The resulting mixture was concentrated under reduced pressure. The residue was purified by silica gel column chromatography, eluted with PE/EtOAc/EtOH (6/3/1). The crude product was purified by prep-HPLC with the following conditions: Column: SunFire Prep C¹⁸ OBD Column, 19×150 mm, 5 μm; Mobile Phase A: water (0.05% FA), Mobile Phase B: CH₃CN; Flow rate: 20 mL/min; Gradient: 55% B to 75% B to afford (3S)-N-[3-[2-methanesulfonamido-6-(morpholin-4-yl)pyridin-4-yl]-4-methylphenyl]-3-(2,2,2-trifluoroethyl)pyrrolidine-1-carboxamide (129 mg, 46%) as an off-white solid. MS ESI calculated for C₂₄H₃₀F₃N₅O₄S [M+H]⁺, 542.20, found 542.20. ¹H NMR (400 MHz, Chloroform-d) δ 7.31-7.28 (m, 2H), 7.18-7.16 (m, 1H), 7.09 (s, 1H), 6.37 (s, 1H), 6.28 (s, 1H), 6.23 (s, 1H), 3.84-3.79 (m, 5H), 3.67-3.62 (m, 1H), 3.52-3.48 (m, 4H), 3.47-3.42 (m, 1H), 3.27 (s, 3H), 3.13 (t, J=9.6 Hz, 1H), 2.57-2.55 (m, 1H), 2.31-2.21 (m, 6H), 1.78-1.73 (m, 1H). ¹⁹F NMR (376 MHz, Chloroform-d) δ-64.94 (3F).

### Example 114: (3S)-N-[3-[2-(3-hydroxy-2-oxopyrrolidin-1-yl)-6-(morpholin-4-yl)pyridin-4-yl]-4-methylphenyl]-3-(2,2,2-trifluoroethyl)pyrrolidine-1-carboxamide

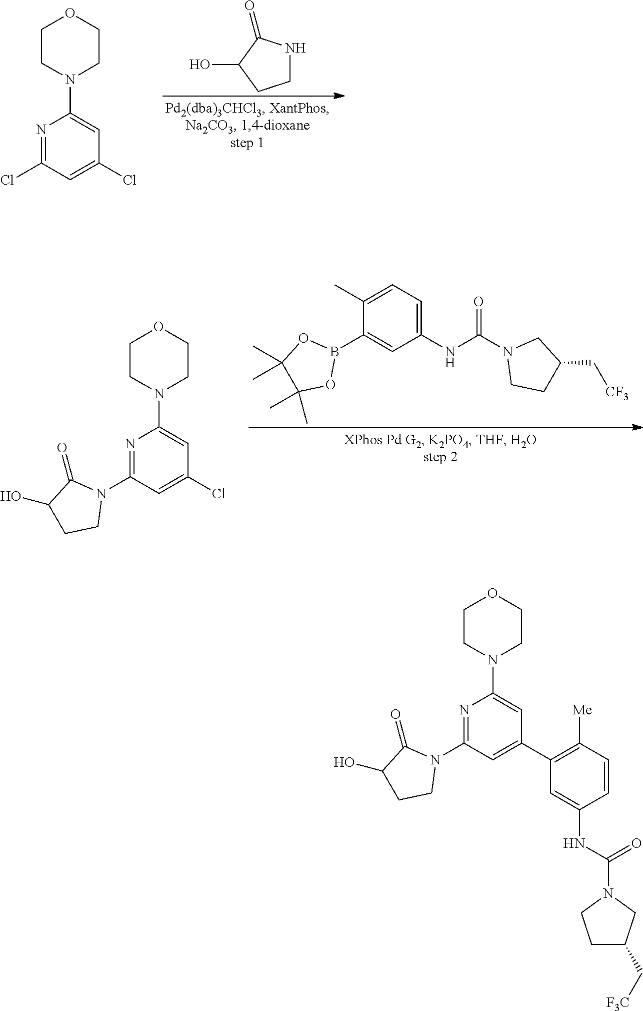 [SMILES: Clc1cc(Cl)nc(N2CCOCC2)c1 ; O=C1NCCC1O ; O=C1C(O)CCN1c1cc(Cl)cc(N2CCOCC2)n1 ; Cc1ccc(NC(=O)N2CC[C@@H](CC(F)(F)F)C2)cc1B1OC(C)(C)C(C)(C)O1 ; Cc1ccc(NC(=O)N2CC[C@@H](CC(F)(F)F)C2)cc1-c1cc(N2CCOCC2)nc(N2CCC(O)C2=O)c1]

Preparation 114A: 1-[4-chloro-6-(morpholin-4-yl)pyridin-2-yl]-3-hydroxypyrrolidin-2-one

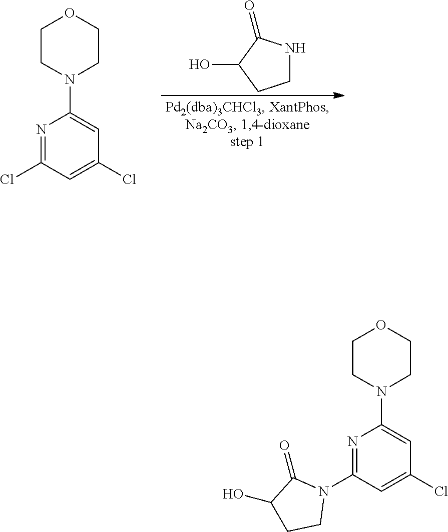 [SMILES: Clc1cc(Cl)nc(N2CCOCC2)c1 ; O=C1NCCC1O ; O=C1C(O)CCN1c1cc(Cl)cc(N2CCOCC2)n1]

To a stirred mixture of 4-(4,6-dichloropyridin-2-yl)morpholine (600 mg, 2.574 mmol), Na₂CO₃ (546 mg, 5.148 mmol) and 3-hydroxypyrrolidin-2-one (390 mg, 3.861 mmol) in 1,4-dioxane (5 mL) were added XantPhos (298 mg, 0.515 mmol) and Pd₂(dba)₃CHCl₃ (266 mg, 0.257 mmol) under nitrogen atmosphere. The resulting mixture was stirred for 16 h at 100° C. under nitrogen atmosphere. The resulting mixture was diluted with H₂O (100 mL). The resulting mixture was extracted with EtOAc (3×50 mL). The combined organic layers were washed with brine (2×50 mL), dried over anhydrous Na₂SO₄. After filtration, the filtrate was concentrated under reduced pressure. The residue was purified by silica gel column chromatography, eluted with PE/EtOAc (3/1) to afford 1-[4-chloro-6-(morpholin-4-yl)pyridin-2-yl]-3-hydroxypyrrolidin-2-one (440 mg, 57%) as a brown solid. MS ESI calculated for C₁₃H₁₆ClN₃O₃[M+H]⁺, 298.09; found 298.05. ¹H NMR (400 MHz, Chloroform-d) δ 7.81 (s, 1H), 6.40-6.39 (m, 1H), 4.55-4.50 (m, 1H), 4.23-4.18 (m, 1H), 3.88-3.68 (m, 5H), 3.57-3.47 (m, 5H), 2.60-2.53 (m, 1H), 2.10-1.76 (m, 1H).

### Example 114: (3S)-N-[3-[2-(3-hydroxy-2-oxopyrrolidin-1-yl)-6-(morpholin-4-yl)pyridin-4-yl]-4-methylphenyl]-3-(2,2,2-trifluoroethyl)pyrrolidine-1-carboxamide

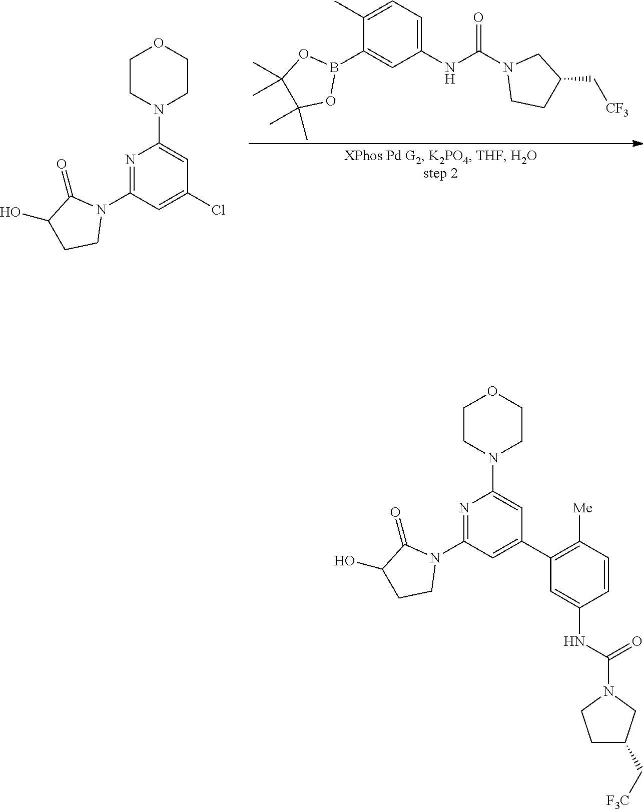 [SMILES: O=C1C(O)CCN1c1cc(Cl)cc(N2CCOCC2)n1 ; Cc1ccc(NC(=O)N2CC[C@@H](CC(F)(F)F)C2)cc1B1OC(C)(C)C(C)(C)O1 ; Cc1ccc(NC(=O)N2CC[C@@H](CC(F)(F)F)C2)cc1-c1cc(N2CCOCC2)nc(N2CCC(O)C2=O)c1]

A mixture of 1-[4-chloro-6-(morpholin-4-yl)pyridin-2-yl]-3-hydroxypyrrolidin-2-one (100 mg, 0.336 mmol), (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (111 mg, 0.269 mmol), 2^{nd} XPhos Precatalyst (26 mg, 0.034 mmol), K₃PO₄ (143 mg, 0.672 mmol), THF (2 mL) and H₂O (0.2 mL) was stirred for 2 h at 80° C. under nitrogen atmosphere. The resulting mixture was diluted with H₂O (100 mL). The resulting mixture was extracted with EtOAc (3×50 mL). The combined organic layers were washed with brine (2×50 mL), dried over anhydrous Na₂SO₄. After filtration, the filtrate was concentrated under reduced pressure. The not pure product was purified by Prep-TLC with PE/EtOAc/EtOH (8/3/1) to afford (3S)-N-[3-[2-(3-hydroxy-2-oxopyrrolidin-1-yl)-6-(morpholin-4-yl)pyridin-4-yl]-4-methylphenyl]-3-(2,2,2-trifluoroethyl)pyrrolidine-1-carboxamide (76 mg, 41%) as an off-white solid. MS ESI calculated for C₂₇H₃₂F₃N₅O₄[M+H]⁺, 548.24; found 548.30. ¹H NMR (300 MHz, Chloroform-d) δ 7.73 (s, 1H), 7.40-7.39 (m, 1H), 7.29-7.26 (m, 1H), 7.21-7.18 (m, 1H), 6.40 (s, 1H), 6.20 (s, 1H), 4.54-4.47 (m, 1H), 4.34-4.26 (m, 1H), 3.87-3.79 (m, 6H), 3.65-3.61 (m, 1H), 3.54-3.43 (m, 5H), 3.16-3.10 (m, 1H), 2.61-2.55 (m, 2H), 2.34-2.25 (m, 6H), 2.09-2.02 (m, 1H), 1.80-1.69 (m, 1H). ¹⁹F NMR (282 MHz, Chloroform-d) δ-64.95 (3F).

### Example 115: (3S)-N-[4-methyl-3-[2-(morpholin-4-yl)-6-(2-oxo-1,3-oxazolidin-3-yl)pyridin-4-yl]phenyl]-3-(2,2,2-trifluoroethyl)pyrrolidine-1-carboxamide

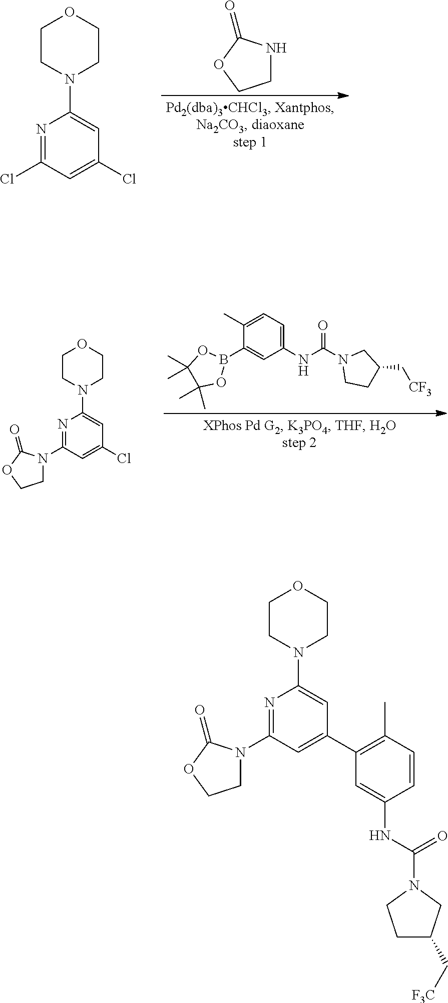 [SMILES: Clc1cc(Cl)nc(N2CCOCC2)c1 ; O=C1NCCO1 ; O=C1OCCN1c1cc(Cl)cc(N2CCOCC2)n1 ; Cc1ccc(NC(=O)N2CC[C@@H](CC(F)(F)F)C2)cc1B1OC(C)(C)C(C)(C)O1 ; Cc1ccc(NC(=O)N2CC[C@@H](CC(F)(F)F)C2)cc1-c1cc(N2CCOCC2)nc(N2CCOC2=O)c1]

Preparation 115A: 3-[4-chloro-6-(morpholin-4-yl)pyridin-2-yl]-1,3-oxazolidin-2-one

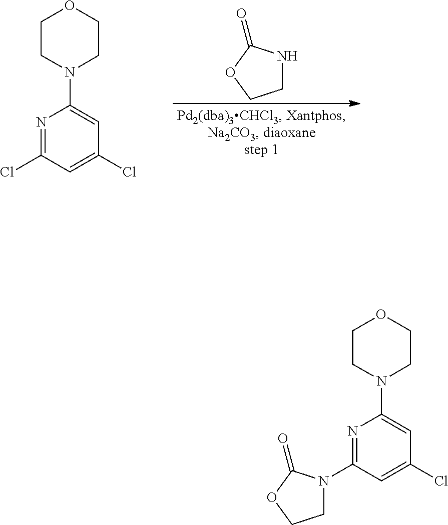 [SMILES: Clc1cc(Cl)nc(N2CCOCC2)c1 ; O=C1NCCO1 ; O=C1OCCN1c1cc(Cl)cc(N2CCOCC2)n1]

To a stirred solution of 4-(4,6-dichloropyridin-2-yl)morpholine (200 mg, 0.858 mmol), Na₂CO₃ (182 mg, 1.716 mmol) and oxazolidinone (112 mg, 1.287 mmol) in 1,4-dioxane (2 mL) was added Pd₂(dba)₃·CHCl₃ (89 mg, 0.086 mmol) and XantPhos (99 mg, 0.172 mmol) under nitrogen atmosphere. The resulting mixture was stirred for 16 h at 100° C. under nitrogen atmosphere. The reaction was quenched by the addition of water (100 mL). The resulting mixture was extracted with EtOAc (3×50 mL). The combined organic layers were washed with saturated brine (3×50 mL), dried over anhydrous Na₂SO₄. After filtration, the filtrate was concentrated under reduced pressure. The residue was purified by silica gel column chromatography, eluted with PE/EtOAc (1/1) to afford 3-[4-chloro-6-(morpholin-4-yl)pyridin-2-yl]-1,3-oxazolidin-2-one (130 mg, 53%) as a brown solid. MS ESI calculated for C₁₂H₁₄ClN₃O₃[M+H]⁺. 284.07, found 284.05. ¹H NMR (400 MHz, Chloroform-d) δ 7.62 (d, J=1.2 Hz, 1H), 6.34 d, J=1.2 Hz, 1H), 4.49-4.45 (m, 2H), 4.25-4.21 (m, 2H), 3.83-3.80 (m, 4H), 3.49-3.47 (m, 4H).

### Example 115: (3S)-N-[4-methyl-3-[2-(morpholin-4-yl)-6-(2-oxo-1,3-oxazolidin-3-yl)pyridin-4-yl]phenyl]-3-(2,2,2-trifluoroethyl)pyrrolidine-1-carboxamide

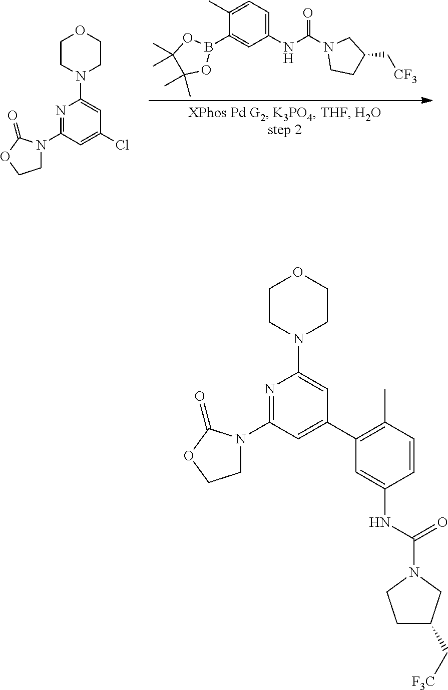 [SMILES: O=C1OCCN1c1cc(Cl)cc(N2CCOCC2)n1 ; Cc1ccc(NC(=O)N2CC[C@@H](CC(F)(F)F)C2)cc1B1OC(C)(C)C(C)(C)O1 ; Cc1ccc(NC(=O)N2CC[C@@H](CC(F)(F)F)C2)cc1-c1cc(N2CCOCC2)nc(N2CCOC2=O)c1]

To a stirred solution of 3-[4-chloro-6-(morpholin-4-yl)pyridin-2-yl]-1,3-oxazolidin-2-one (130 mg, 0.458 mmol), (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (189 mg, 0.458 mmol) and K₃PO₄ (195 mg, 0.916 mmol) in THF (2 mL) and H₂O (0.2 mL) was added 2^{nd} XPhos Precatalyst (36 mg, 0.046 mmol) under nitrogen atmosphere. The resulting mixture was stirred for 2 h at 80° C. under nitrogen atmosphere. The reaction was quenched by the addition of water (100 mL). The resulting mixture was extracted with EtOAc (3×50 mL). The combined organic layers were washed with saturated brine (3×50 mL), dried over anhydrous Na₂SO₄. After filtration, the filtrate was concentrated under reduced pressure. The residue was purified by silica gel column chromatography, eluted with PE/EtOAc (1/1). The crude product was purified by reverse flash chromatography with the following conditions: column, C18 silica gel; mobile phase, CH₃CN in water (10 mmol/L NH₄HCO₃), 5% to 95% gradient; detector, UV 254 nm to afford (3S)-N-[4-methyl-3-[2-(morpholin-4-yl)-6-(2-oxo-1,3-oxazolidin-3-yl)pyridin-4-yl]phenyl]-3-(2,2,2-trifluoroethyl)pyrrolidine-1-carboxamide (122 mg, 50%) as an off-white solid. MS ESI calculated for C₂₆H₃₀F₃N₅O₄ [M+H]⁺. 534.22, found 534.30. ¹H NMR (300 MHz, Chloroform-d) δ 7.52 (s, 1H), 7.38 (dd, J=2.4, 8.4 Hz, 1H), 7.22 (d, J=2.1 Hz, 1H), 7.16 (d, J=8.4 Hz, 1H), 6.32 (s, 1H), 6.18 (s, 1H), 4.49-4.44 (m, 2H), 4.31-4.26 (m, 2H), 3.84-3.76 (m, 5H), 3.65-3.59 (m, 1H), 3.50-3.38 (m, 5H), 3.13-3.07 (m, 1H), 2.60-2.50 (m, 1H), 2.31-2.18 (m, 6H), 1.77-1.73 (m, 1H). ¹⁹F NMR (282 MHz, Chloroform-d) δ-64.95 (3F).

### Example 116: (3S)-N-(3-[2-[5-hydroxy-2-oxopiperidin-1-yl]-6-(morpholin-4-yl)pyridin-4-yl]-4-methylphenyl)-3-(2,2,2-trifluoroethyl)pyrrolidine-1-carboxamide

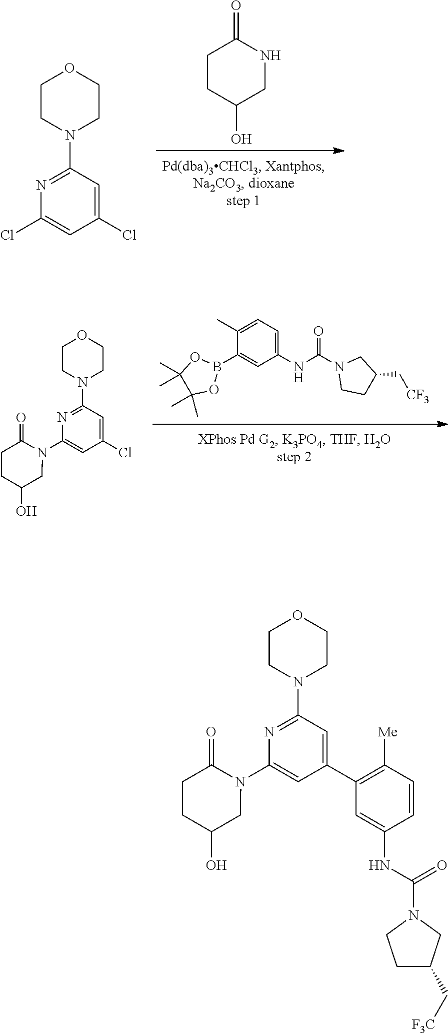 [SMILES: Clc1cc(Cl)nc(N2CCOCC2)c1 ; O=C1CCC(O)CN1 ; O=C1CCC(O)CN1c1cc(Cl)cc(N2CCOCC2)n1 ; Cc1ccc(NC(=O)N2CC[C@@H](CC(F)(F)F)C2)cc1B1OC(C)(C)C(C)(C)O1 ; Cc1ccc(NC(=O)N2CC[C@@H](CC(F)(F)F)C2)cc1-c1cc(N2CCOCC2)nc(N2CC(O)CCC2=O)c1]

Preparation 116A: tert-butyl 1-[4-chloro-6-(morpholin-4-yl)pyridin-2-yl]-5-hydroxypiperidin-2-one

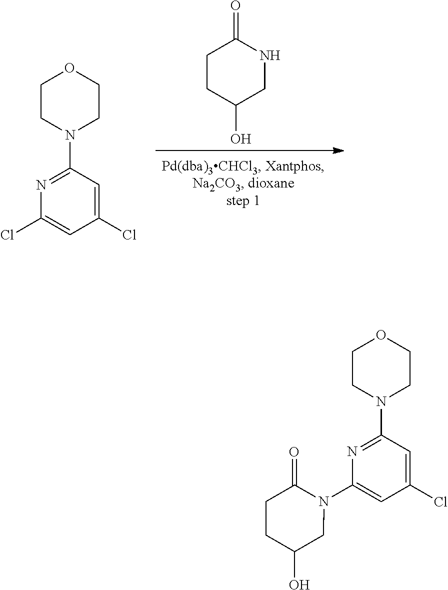 [SMILES: Clc1cc(Cl)nc(N2CCOCC2)c1 ; O=C1CCC(O)CN1 ; O=C1CCC(O)CN1c1cc(Cl)cc(N2CCOCC2)n1]

To a mixture of 4-(4,6-dichloropyridin-2-yl)morpholine (500 mg, 2.145 mmol) and 5-hydroxypiperidin-2-one (370 mg, 3.218 mmol) in dioxane (7 mL) were added XantPhos (248 mg, 0.429 mmol) and Na₂CO₃ (455 mg, 4.290 mmol) and Pd₂(dba)₃-CHCl₃ (222 mg, 0.215 mmol). The resulting mixture was stirred for 16 h at 100° C. under nitrogen atmosphere. The resulting mixture was diluted with water (15 mL). The resulting mixture was extracted with EtOAc (3×15 mL). The combined organic layers were washed with brine (2×10 mL), dried over anhydrous Na₂SO₄. After filtration, the filtrate was concentrated under reduced pressure. The residue was purified by silica gel column chromatography, eluted with PE/EtOAc (1/1) to afford 1-[4-chloro-6-(morpholin-4-yl)pyridin-2-yl]-5-hydroxypiperidin-2-one (498 mg, 74%) as an off-white solid. MS ESI calculated for C₁₄H₁₈ClN₃O₃[M+H]⁺. 312.10, found 312.05. ¹H NMR (300 MHz, DMSO-d₆) δ 7.29 (d, J=1.2 Hz, 1H), 6.70 (d, J=1.5 Hz, 1H), 5.09 (d, J=3.6 Hz, 1H), 4.09 (s, 1H), 3.90-3.81 (m, 1H), 3.79-3.75 (m, 1H), 3.71-3.68 (m, 4H), 3.48-3.45 (m, 4H), 2.62-2.40 (m, 2H), 1.98-1.97 (m, 1H), 1.80-1.73 (m, 1H).

### Example 116: (3S)-N-[3-[2-(5-hydroxy-2-oxopiperidin-1-yl)-6-(morpholin-4-yl)pyridin-4-yl]-4-methylphenyl]-3-(2,2,2-trifluoroethyl)pyrrolidine-1-carboxamide

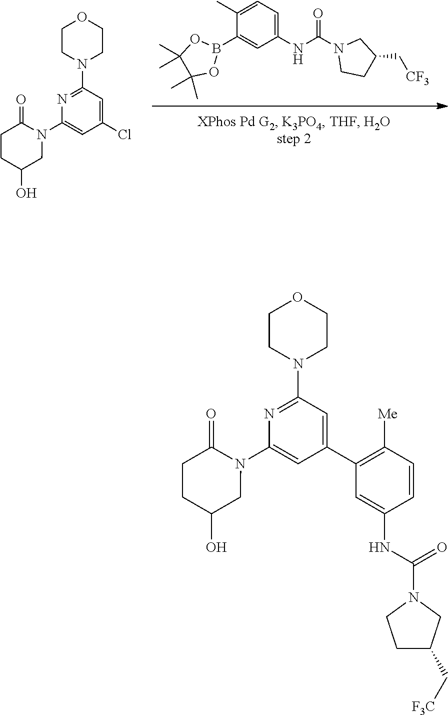 [SMILES: O=C1CCC(O)CN1c1cc(Cl)cc(N2CCOCC2)n1 ; Cc1ccc(NC(=O)N2CC[C@@H](CC(F)(F)F)C2)cc1B1OC(C)(C)C(C)(C)O1 ; Cc1ccc(NC(=O)N2CC[C@@H](CC(F)(F)F)C2)cc1-c1cc(N2CCOCC2)nc(N2CC(O)CCC2=O)c1]

To a stirred mixture of 1-[4-chloro-6-(morpholin-4-yl)pyridin-2-yl]-5-hydroxypiperidin-2-one (250 mg, 0.802 mmol) and (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (264 mg, 0.641 mmol) in THF (8 mL) and H₂O (0.8 mL) were added K₃PO₄ (340 mg, 1.604 mmol) and 2^{nd} XPhos Precatalyst (63 mg, 0.080 mmol). The resulting mixture was stirred for 2 h at 80° C. under nitrogen atmosphere. The resulting mixture was diluted with water (30 mL). The resulting mixture was extracted with EtOAc (3×20 mL). The combined organic layers were washed with brine (2×10 mL), dried over anhydrous Na₂SO₄. After filtration, the filtrate was concentrated under reduced pressure. The residue was purified by silica gel column chromatography, eluted with PE/EtOAc (1/1) to afford (3S)-N-[3-[2-(5-hydroxy-2-oxopiperidin-1-yl)-6-(morpholin-4-yl)pyridin-4-yl]-4-methylphenyl]-3-(2,2,2-trifluoroethyl)pyrrolidine-1-carboxamide (274 mg, 61%) as an off-white solid. MS ESI calculated for C₂₈H₃₄F₃N₅O₄[M+H]⁺ 562.26, found 562.20.

### Example 117: (3S)-N-(4-methyl-3-(2-morpholino-6-(2-oxo-3-azabicyclo[3.1.0]hexan-3-yl)pyridin-4-yl)phenyl)-3-(2,2,2-trifluoroethyl)pyrrolidine-1-carboxamide

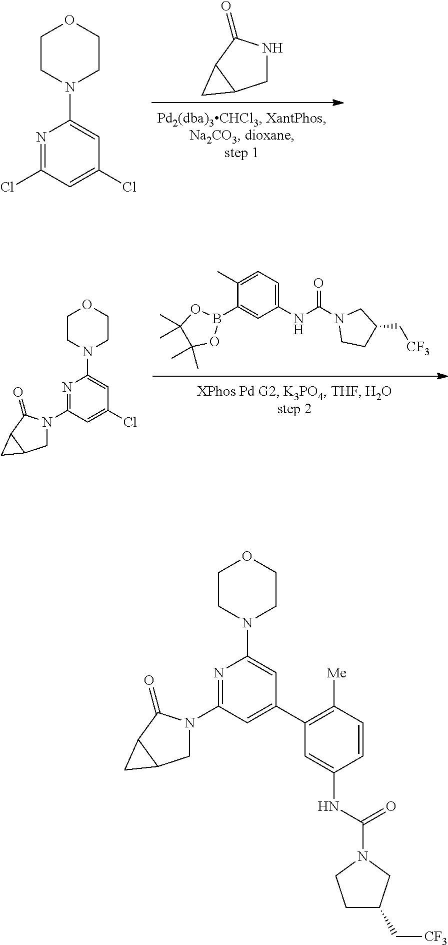 [SMILES: Clc1cc(Cl)nc(N2CCOCC2)c1 ; O=C1NCC2CC12 ; O=C1C2CC2CN1c1cc(Cl)cc(N2CCOCC2)n1 ; Cc1ccc(NC(=O)N2CC[C@@H](CC(F)(F)F)C2)cc1B1OC(C)(C)C(C)(C)O1 ; Cc1ccc(NC(=O)N2CC[C@@H](CC(F)(F)F)C2)cc1-c1cc(N2CCOCC2)nc(N2CC3CC3C2=O)c1]

Preparation 117A: 3-[4-chloro-6-(morpholin-4-yl)pyridin-2-yl]-3-azabicyclo[3.1.0]hexan-2-one

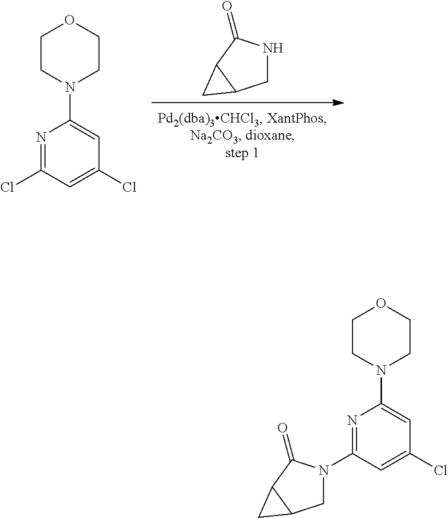 [SMILES: Clc1cc(Cl)nc(N2CCOCC2)c1 ; O=C1NCC2CC12 ; O=C1C2CC2CN1c1cc(Cl)cc(N2CCOCC2)n1]

A mixture of 4-(4,6-dichloropyridin-2-yl)morpholine (500 mg, 2.145 mmol), 3-azabicyclo[3.1.0]hexan-2-one (187 mg, 1.931 mmol), Pd₂(dba)₃·CHCl₃ (222 mg, 0.215 mmol), XantPhos (248 mg, 0.429 mmol) and Na₂CO₃ (455 mg, 4.290 mmol) in dioxane (5 mL) was stirred for 3 h at 100° C. under nitrogen atmosphere. The resulting mixture was concentrated under reduced pressure. The residue was purified by silica gel column chromatography, eluted with PE/EtOAc (1/1) to afford 3-[4-chloro-6-(morpholin-4-yl)pyridin-2-yl]-3-azabicyclo[3.1.0]hexan-2-one (810 mg, 86%) as a red solid. MS ESI calculated for C₁₄H₁₆ClN₃O₂[M+H]⁺, 294.09, found 294.05. ¹H NMR (400 MHz, Chloroform-d) δ 7.74 (s, 1H), 6.32 (s, 1H), 4.07-4.03 (m, 2H), 3.82-3.80 (m, 4H), 3.47-3.45 (m, 4H), 2.15-2.11 (m, 1H), 2.01-1.95 (m, 1H), 1.24-1.19 (m, 1H), 0.80-0.77 (m, 1H).

### Example 117: (3S)-N-[4-methyl-3-[2-(morpholin-4-yl)-6-[2-oxo-3-azabicyclo[3.1.0]hexan-3-yl]pyridin-4-yl]phenyl]-3-(2,2,2-trifluoroethyl)pyrrolidine-1-carboxamide

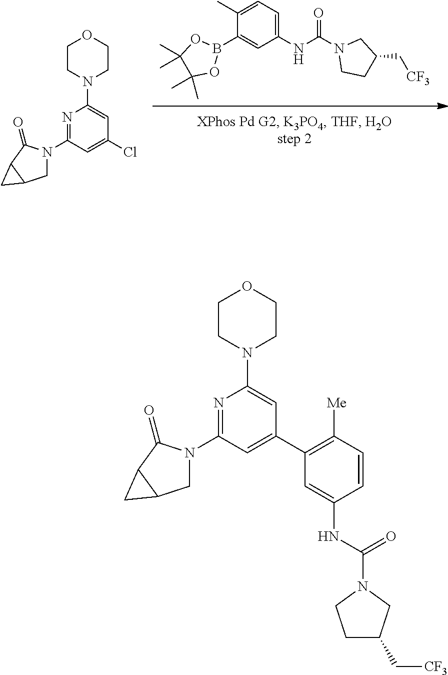 [SMILES: O=C1C2CC2CN1c1cc(Cl)cc(N2CCOCC2)n1 ; Cc1ccc(NC(=O)N2CC[C@@H](CC(F)(F)F)C2)cc1B1OC(C)(C)C(C)(C)O1 ; Cc1ccc(NC(=O)N2CC[C@@H](CC(F)(F)F)C2)cc1-c1cc(N2CCOCC2)nc(N2CC3CC3C2=O)c1]

A mixture of 3-[4-chloro-6-(morpholin-4-yl)pyridin-2-yl]-3-azabicyclo[3.1.0]hexan-2-one (200 mg, 0.681 mmol), (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (225 mg, 0.545 mmol), 2^{nd} XPhos Precatalyst (54 mg, 0.068 mmol) and K₃PO₄ (289 mg, 1.362 mmol) in THF (2 mL) and H₂O (0.2 mL) was stirred for 2 h at 80° C. under nitrogen atmosphere. The reaction was quenched by the addition of water (10 mL). The resulting mixture was extracted with EtOAc (2×5 mL). The combined organic layers were washed with brine (5 mL), dried over anhydrous Na₂SO₄. After filtration, the filtrate was concentrated under reduced pressure. The residue was purified by silica gel column chromatography, eluted with PE/EtOAc (1/1) to afford (3S)-N-[4-methyl-3-[2-(morpholin-4-yl)-6-[2-oxo-3-azabicyclo[3.1.0]hexan-3-yl]pyridin-4-yl]phenyl]-3-(2,2,2-trifluoroethyl)pyrrolidine-1-carboxamide (109 mg, 29%) as an off-white solid. MS ESI calculated for C₂₈H₃₂F₃N₅O₃[M+H]⁺, 544.25, found 544.35. ¹H NMR (300 MHz, Chloroform-d) δ 7.63 (s, 1H), 7.40 (dd, J=2.1, 8.1 Hz, 1H), 7.26-7.14 (m, 2H), 6.27 (s, 1H), 6.18 (s, 1H), 4.13 (s, 1H), 4.11 (s, 1H), 3.83-3.76 (m, 5H), 3.61-3.58 (m, 1H), 3.48-3.38 (m, 5H), 3.13-3.07 (m, 1H), 2.31-2.28 (m, 1H), 2.25-2.22 (m, 6H), 2.18-2.06 (m, 1H), 1.98-1.95 (m, 1H), 1.76-1.73 (m, 1H), 1.21-1.15 (m, 1H), 0.81-0.77 (m, 1H). ¹⁹F NMR (282 MHz, Chloroform-d) δ-64.94 (3F).

### Example 118: (3S)-N-(3-[2-[(3S,4S)-3,4-dihydroxypyrrolidin-1-yl]-6-(morpholin-4-yl)pyridin-4-yl]-4-methylphenyl)-3-(2,2,2-trifluoroethyl)pyrrolidine-1-carboxamide

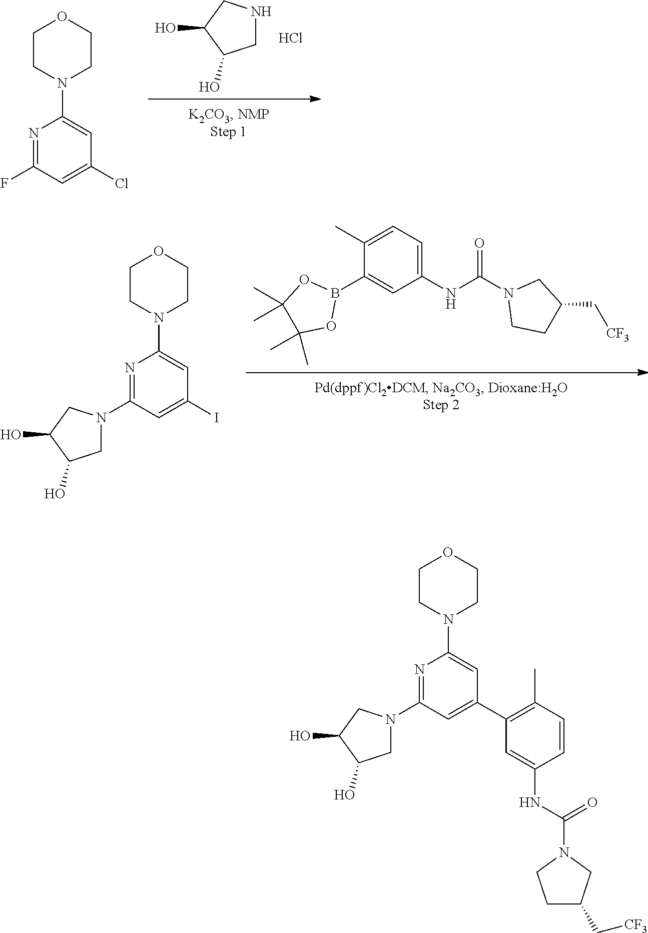 [SMILES: Fc1cc(Cl)cc(N2CCOCC2)n1 ; O[C@H]1CNC[C@@H]1O ; Cl ; O[C@H]1CN(c2cc(I)cc(N3CCOCC3)n2)C[C@@H]1O ; Cc1ccc(NC(=O)N2CC[C@@H](CC(F)(F)F)C2)cc1B1OC(C)(C)C(C)(C)O1 ; Cc1ccc(NC(=O)N2CC[C@@H](CC(F)(F)F)C2)cc1-c1cc(N2CCOCC2)nc(N2C[C@H](O)[C@@H](O)C2)c1]

Preparation 118A: (3S,4S)-1-[4-iodo-6-(morpholin-4-yl)pyridin-2-yl]pyrrolidine-3,4-diol

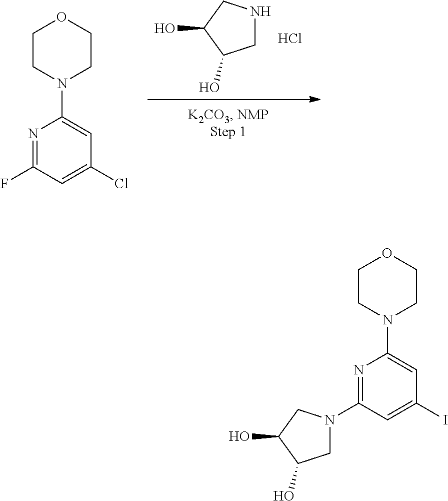 [SMILES: Fc1cc(Cl)cc(N2CCOCC2)n1 ; O[C@H]1CNC[C@@H]1O ; Cl ; O[C@H]1CN(c2cc(I)cc(N3CCOCC3)n2)C[C@@H]1O]

A mixture of 4-(6-fluoro-4-iodopyridin-2-yl)morpholine (200 mg, 0.649 mmol), K₂CO₃ (358 mg, 2.597 mmol) and (3S,4S)-pyrrolidine-3,4-diol hydrochloride (181 mg, 1.298 mmol) in NMP (1.6 mL) was stirred for 16 h at 100° C. under nitrogen atmosphere. The resulting mixture was diluted with water (100 mL). The resulting mixture was extracted with EtOAc (3×100 mL). The combined organic layers were washed with brine (2×100 mL), dried over anhydrous Na₂SO₄. After filtration, the filtrate was concentrated under reduced pressure. The residue was purified by silica gel column chromatography, eluted with hexane/EtOAc/EtOH (4/3/1) to afford (3S,4S)-1-[4-iodo-6-(morpholin-4-yl)pyridin-2-yl]pyrrolidine-3,4-diol (237 mg, 93%) as a white solid. MS ESI calculated for C₁₃H₁₈IN₃O₃[M+H]⁺, 392.04 found 391.95. ¹H NMR (300 MHz, DMSO-d₆) δ 6.31 (s, 1H), 6.10 (s, 1H), 5.06 (s, 2H), 3.99-3.98 (m, 2H), 3.68-3.65 (m, 4H), 3.50-3.49 (m, 2H), 3.46-3.45 (m, 4H), 3.27-3.23 (m, 2H).

### Example 118: (3S)-N-(3-[2-[(3S,4S)-3,4-dihydroxypyrrolidin-1-yl]-6-(morpholin-4-yl)pyridin-4-yl]-4-methylphenyl)-3-(2,2,2-trifluoroethyl)pyrrolidine-1-carboxamide

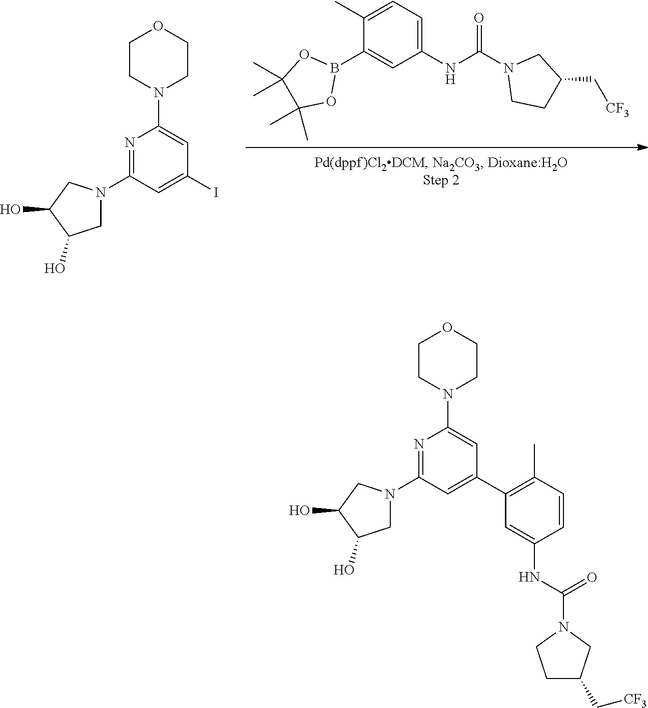 [SMILES: O[C@H]1CN(c2cc(I)cc(N3CCOCC3)n2)C[C@@H]1O ; Cc1ccc(NC(=O)N2CC[C@@H](CC(F)(F)F)C2)cc1B1OC(C)(C)C(C)(C)O1 ; Cc1ccc(NC(=O)N2CC[C@@H](CC(F)(F)F)C2)cc1-c1cc(N2CCOCC2)nc(N2C[C@H](O)[C@@H](O)C2)c1]

A mixture of (3S,4S)-1-[4-iodo-6-(morpholin-4-yl)pyridin-2-yl]pyrrolidine-3,4-diol (120 mg, 0.307 mmol), (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (113 mg, 0.276 mmol), Na₂CO₃ (98 mg, 0.920 mmol), dioxane (3 mL), H₂O (0.75 mL) and Pd(dppfCl₂CH₂Cl₂ (25 mg, 0.031 mmol) was stirred for 3 h at 60° C. under nitrogen atmosphere. The resulting mixture was diluted with water (50 mL). The resulting mixture was extracted with EtOAc (3×50 mL). The combined organic layers were washed with brine (2×50 mL), dried over anhydrous Na₂SO₄. After filtration, the filtrate was concentrated under reduced pressure. The residue was purified by Prep-TLC with CH₂Cl₂/CH₃OH (10:1) to afford (3S)-N-(3-[2-[(3S,4S)-3,4-dihydroxypyrrolidin-1-yl]-6-(morpholin-4-yl)pyridin-4-yl]-4-methylphenyl)-3-(2,2,2-trifluoroethyl)pyrrolidine-1-carboxamide (76.3 mg, 45%) as an off-white solid. MS ESI calculated for C₂₇H₃₄F₃N₅O₄ [M+H]⁺, 550.26 found 550.30. ¹H NMR (400 MHz, DMSO-d₆) δ 8.12 (s, 1H), 7.43 (dd, J=2.0, 8.4 Hz, 1H), 7.34 (d, J=2.0 Hz, 1H), 7.10 (d, J=8.4 Hz, 1H), 5.84 (s, 1H), 5.62 (s, 1H), 5.04 (d, J=3.2 Hz, 2H), 4.01 (s, 2H), 3.70-3.65 (m, 5H), 3.54-3.51 (m, 3H), 3.42-3.40 (m, 4H), 3.30-3.29 (m, 3H), 3.05-3.00 (m, 1H), 2.48-2.41 (m, 3H), 2.17 (s, 3H), 2.10-2.07 (m, 1H), 1.69-1.62 (m, 1H). ¹⁹F NMR (376 MHz, DMSO-d₆) δ-63.36 (3F).

### Example 119: (3S)-N-(3-[2-[(3S,4S)-3-fluoro-4-hydroxypyrrolidin-1-yl]-6-(morpholin-4-yl)pyridin-4-yl]-4-methylphenyl)-3-(2,2,2-trifluoroethyl)pyrrolidine-1-carboxamide

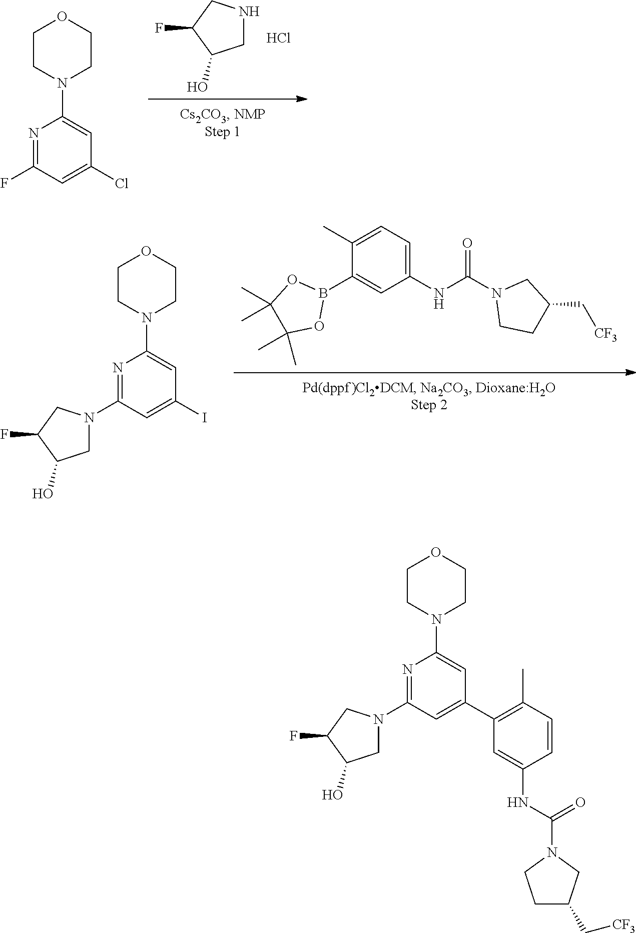 [SMILES: Fc1cc(Cl)cc(N2CCOCC2)n1 ; O[C@H]1CNC[C@@H]1F ; Cl ; O[C@H]1CN(c2cc(I)cc(N3CCOCC3)n2)C[C@@H]1F ; Cc1ccc(NC(=O)N2CC[C@@H](CC(F)(F)F)C2)cc1B1OC(C)(C)C(C)(C)O1 ; Cc1ccc(NC(=O)N2CC[C@@H](CC(F)(F)F)C2)cc1-c1cc(N2CCOCC2)nc(N2C[C@H](O)[C@@H](F)C2)c1]

Preparation 119A: (3S,4S)-4-fluoro-1-[4-iodo-6-(morpholin-4-yl)pyridin-2-yl]pyrrolidin-3-ol

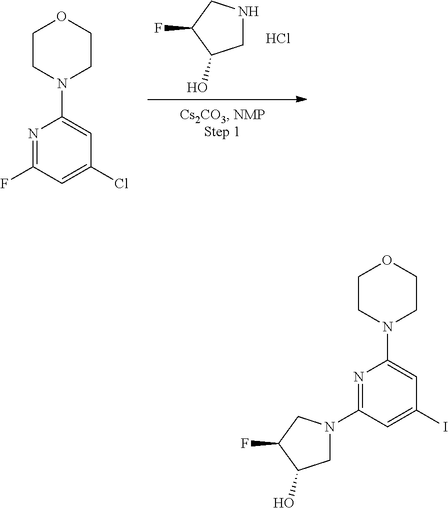 [SMILES: Fc1cc(Cl)cc(N2CCOCC2)n1 ; O[C@H]1CNC[C@@H]1F ; Cl ; O[C@H]1CN(c2cc(I)cc(N3CCOCC3)n2)C[C@@H]1F]

To a stirred mixture of 4-(6-fluoro-4-iodopyridin-2-yl)morpholine (300 mg, 0.974 mmol) and (3S,4S)-4-fluoropyrrolidin-3-ol hydrochloride (165 mg, 1.168 mmol) in NMP (3 mL) was added Cs₂CO₃ (635 mg, 1.947 mmol) was stirred for 2 h in portions at 130° C. under nitrogen atmosphere. The reaction was quenched by the addition of water (20 mL) at room temperature. The resulting mixture was extracted with EtOAc (3×20 mL). The combined organic layers were washed with brine (2×5 mL), dried over anhydrous Na₂SO₄. After filtration, the filtrate was concentrated under reduced pressure. The residue was purified by silica gel column chromatography, eluted with PE/EtOAc (5/1) to afford (3S,4S)-4-fluoro-1-[4-iodo-6-(morpholin-4-yl)pyridin-2-yl]pyrrolidin-3-ol (113 mg, 29%) as a light yellow solid. MS ESI calculated for C₁₃H₁₇FIN₃O₂[M+H]⁺, 394.04 found 394.00. ¹H NMR (400 MHz, Chloroform-d) δ 6.32 (s, 1H), 6.19 (s, 1H), 5.06 (d, J=51.2 Hz, 1H), 4.55-4.51 (m, 1H), 3.85-3.66 (m, 7H), 3.62-3.58 (m, 1H), 3.52-3.44 (m, 4H).

### Example 119: (3S)-N-(3-[2-[(3S,4S)-3-fluoro-4-hydroxypyrrolidin-1-yl]-6-(morpholin-4-yl)pyridin-4-yl]-4-methylphenyl)-3-(2,2,2-trifluoroethyl)pyrrolidine-1-carboxamide

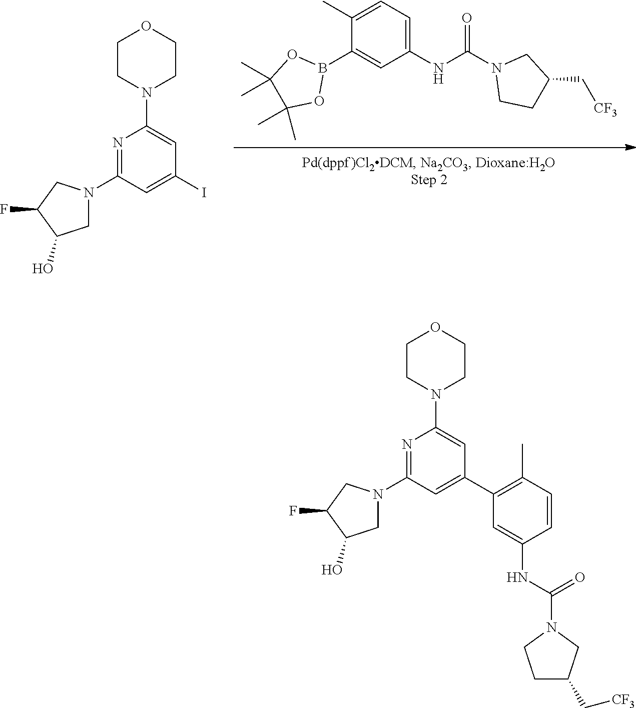 [SMILES: O[C@H]1CN(c2cc(I)cc(N3CCOCC3)n2)C[C@@H]1F ; Cc1ccc(NC(=O)N2CC[C@@H](CC(F)(F)F)C2)cc1B1OC(C)(C)C(C)(C)O1 ; Cc1ccc(NC(=O)N2CC[C@@H](CC(F)(F)F)C2)cc1-c1cc(N2CCOCC2)nc(N2C[C@H](O)[C@@H](F)C2)c1]

A mixture of (3S,4S)-4-fluoro-1-[4-iodo-6-(morpholin-4-yl)pyridin-2-yl]pyrrolidin-3-ol (90 mg, 0.218 mmol), (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (90 mg, 0.218 mmol), 1,4-dioxane (1.8 mL), H₂O (0.45 mL), Na₂CO₃ (69 mg, 0.653 mmol) and Pd(dppf)Cl₂·CH₂Cl₂ (18 mg, 0.022 mmol) was stirred for 16 h at 60° C. under nitrogen atmosphere. The reaction was quenched by the addition of water (5 mL). The resulting mixture was extracted with EtOAc (3×20 mL). The combined organic layers were washed with brine (3×4 mL), dried over anhydrous Na₂SO₄. After filtration, the filtrate was concentrated under reduced pressure. The residue was purified by silica gel column chromatography, eluted with PE/EtOAc (5/1) to afford (3S)-N-(3-[2-[(3S,4S)-3-fluoro-4-hydroxypyrrolidin-1-yl]-6-(morpholin-4-yl)pyridin-4-yl]-4-methylphenyl)-3-(2,2,2-trifluoroethyl)pyrrolidine-1-carboxamide (74.9 mg, 62%) as an off-white solid. MS ESI calculated for C₂₇H₃₃F₄N₅O₃[M+H]⁺, 552.25 found 552.35. ¹H NMR (400 MHz, Chloroform-d) δ 7.37 (dd, J=2.4, 8.4 Hz, 1H), 7.23 (d, J=2.4 Hz, 1H), 7.18 (d, J=8.4 Hz, 1H), 6.21 (s, 1H), 5.90 (s, 1H), 5.74 (s, 1H), 5.07 (d, J=50.8 Hz, 1H), 4.52-4.50 (m, 1H), 3.92-3.71 (m, 8H), 3.65-3.61 (m, 2H), 3.52-3.50 (m, 4H), 3.46-3.41 (m, 1H), 3.11-3.07 (m, 1H), 2.61-2.49 (m, 1H), 2.33-2.20 (m, 6H), 1.78-1.70 (m, 1H), 1.70-1.66 (m, 1H). ¹⁹F NMR (376 MHz, Chloroform-d) δ-63.93 (3F), −182.75 (1F).

### Example 120: (3S)-N-[3-[2-cyclopropanesulfonamido-6-(morpholin-4-yl)pyridin-4-yl]-4-methylphenyl]-3-(2,2,2 trifluoroethyl)pyrrolidine-1-carboxamide

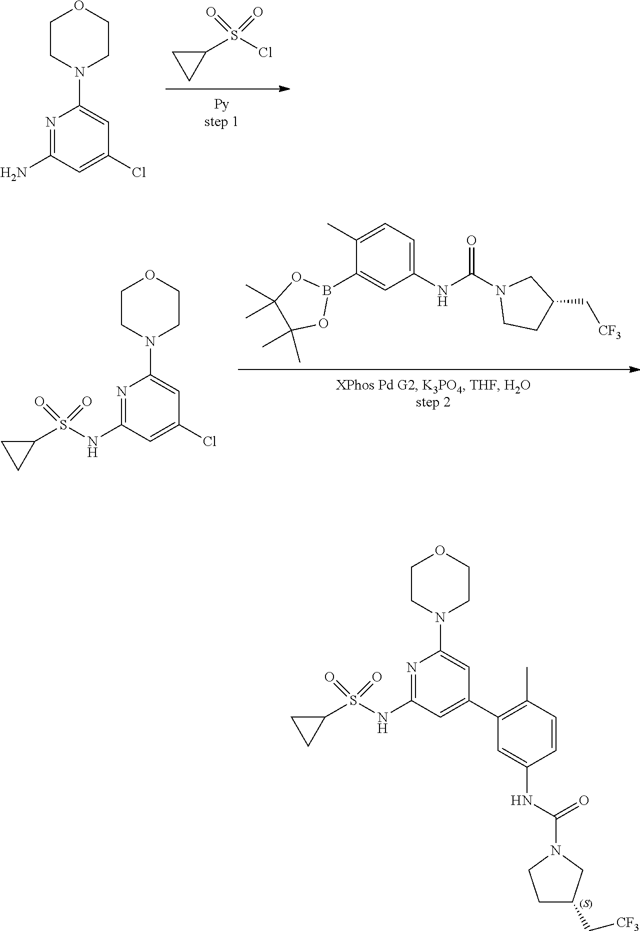

Preparation 120A: N-[4-chloro-6-(morpholin-4-yl)pyridin-2-yl]cyclopropanesulfonamide

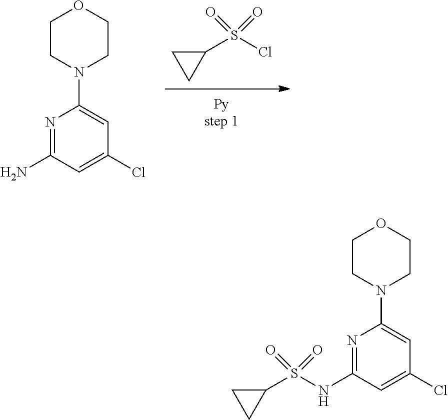 [SMILES: Nc1cc(Cl)cc(N2CCOCC2)n1 ; O=S(=O)(Cl)C1CC1 ; O=S(=O)(Nc1cc(Cl)cc(N2CCOCC2)n1)C1CC1]

A mixture of 4-chloro-6-(morpholin-4-yl)pyridin-2-amine (200 mg, 0.936 mmol) in pyridine (6 mL) and cyclopropanesulfonyl chloride (197 mg, 1.404 mmol) was stirred for 48 h at room temperature. The reaction mixture was concentrated under reduced pressure. The residue was purified by reverse flash chromatography with the following conditions: column, C₁₈ silica gel; mobile phase, CH₃CN in water (with 10 mmol/L NH₄HCO₃), gradient 10% to 95%; detector, UV 254 nm to afford N-[4-chloro-6-(morpholin-4-yl)pyridin-2-yl]cyclopropanesulfonamide (127 mg, 17%) as an off-white solid. MS ESI calculated for C₁₂H₁₆ClN₃O₃S [M+H]⁺, 318.06, found 318.10. ¹H NMR (400 MHz, Chloroform-d) δ 6.65 (s, 1H), 6.34 (s, 1H), 3.91-3.71 (m, 4H), 3.59-3.39 (m, 4H), 2.80-2.76 m, 1H), 1.41-1.23 (m, 2H), 1.19-0.97 (m, 2H).

### Example 120: (3S)-N-[3-[2-cyclopropanesulfonamido-6-(morpholin-4-yl)pyridin-4-yl]-4-methylphenyl]-3-(2,2,2 trifluoroethyl)pyrrolidine-1-carboxamide

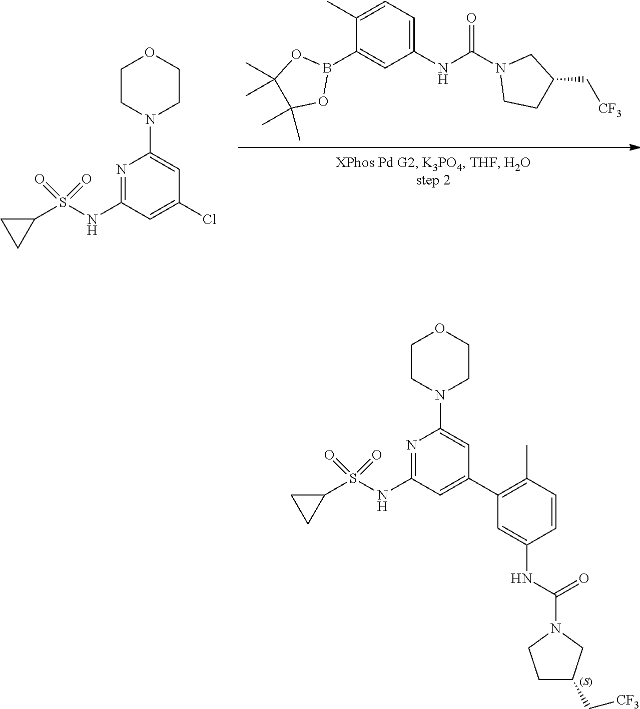

A mixture of N-[4-chloro-6-(morpholin-4-yl)pyridin-2-yl]cyclopropanesulfonamide (100 mg, 0.300 mmol), (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (116.8 mg, 0.300 mmol), 2^{nd} XPhos Precatalyst (24 mg, 0.030 mmol) and K₃PO₄ (135 mg, 0.640 mmol) in THF (5 mL) and H₂O (0.5 mL) was stirred for 2 h at 80° C. under nitrogen atmosphere. The resulting mixture was concentrated under reduced pressure. The residue was purified by silica gel column chromatography, eluted with PE/EtOAc (1/1) to afford (3S)-N-[3-[2-cyclopropanesulfonamido-6-(morpholin-4-yl)pyridin-4-yl]-4-methylphenyl]-3-(2,2,2 trifluoroethyl)pyrrolidine-1-carboxamide (69 mg, 38%) as an off-white solid. MS ESI calculated for C₂₆H₃₂F₃N₅O₄S [M+H]⁺, 568.21, found 568.15. ¹H NMR (400 MHz, DMSO-d₆) δ 10.29 (s, 1H), 8.17 (s, 1H), 7.46 (dd, J=8.4, 2.4 Hz, 1H), 7.37 (d, J=2.4 Hz, 1H), 7.14 (d, J=8.4 Hz, 1H), 6.32 (s, 1H), 6.18 (s, 1H), 3.86-3.62 (m, 5H), 3.60-3.41 (m, 5H), 3.32-3.27 (m, 1H), 3.17-2.93 (m, 2H), 2.50-2.29 (m, 3H), 2.16 (s, 3H), 2.09-2.07 (m, 1H), 1.74-1.55 (m, 1H), 1.04-1.02 (m, 4H). ¹⁹F NMR (282 MHz, DMSO-d₆) δ-63.36 (3F).

### Example 121: (3S)-N-(3-[2-[(3S)-3-amino-2-oxopyrrolidin-1-yl]-6-(morpholin-4-yl)pyridin-4-yl]-4-methylphenyl)-3-(2,2,2-trifluoroethyl)pyrrolidine-1-carboxamide

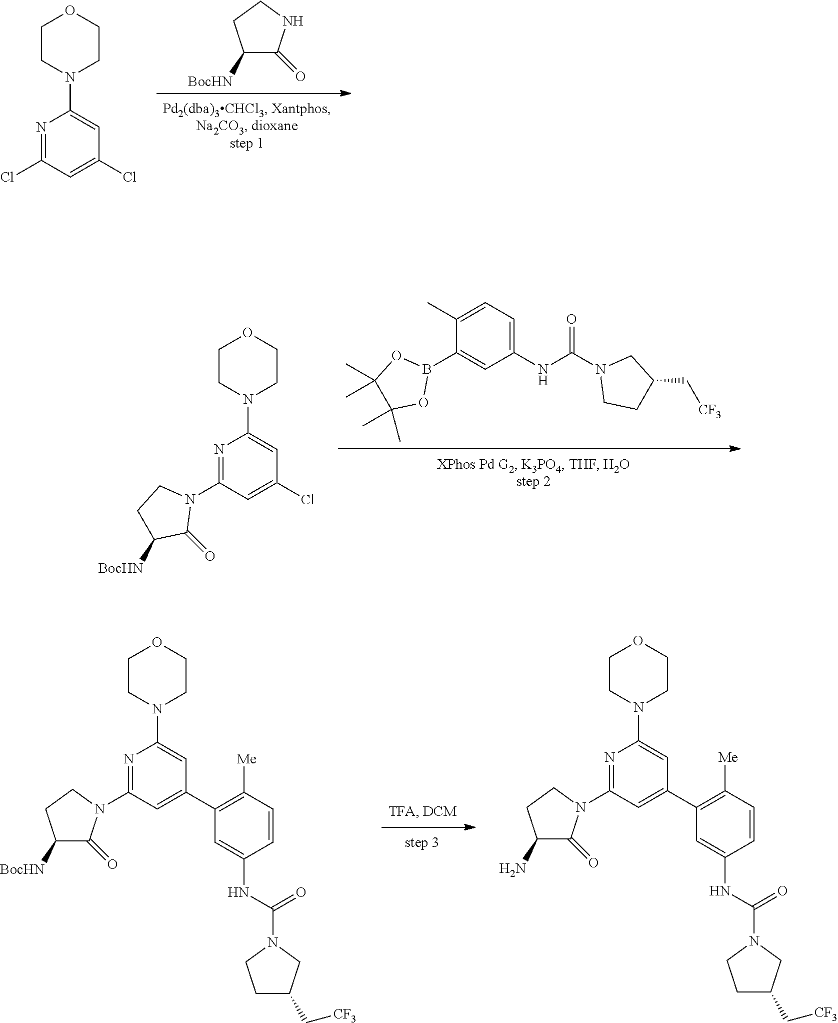 [SMILES: Clc1cc(Cl)nc(N2CCOCC2)c1 ; CC(C)(C)OC(=O)N[C@H]1CCNC1=O ; CC(C)(C)OC(=O)N[C@H]1CCN(c2cc(Cl)cc(N3CCOCC3)n2)C1=O ; Cc1ccc(NC(=O)N2CC[C@@H](CC(F)(F)F)C2)cc1B1OC(C)(C)C(C)(C)O1 ; Cc1ccc(NC(=O)N2CC[C@@H](CC(F)(F)F)C2)cc1-c1cc(N2CCOCC2)nc(N2CC[C@H](NC(=O)OC(C)(C)C)C2=O)c1 ; Cc1ccc(NC(=O)N2CC[C@@H](CC(F)(F)F)C2)cc1-c1cc(N2CCOCC2)nc(N2CC[C@H](N)C2=O)c1]

Preparation 121A: tert-butyl N-[(3S)-1-[4-chloro-6-(morpholin-4-yl)pyridin-2-yl]-2-oxopyrrolidin-3-yl]carbamate

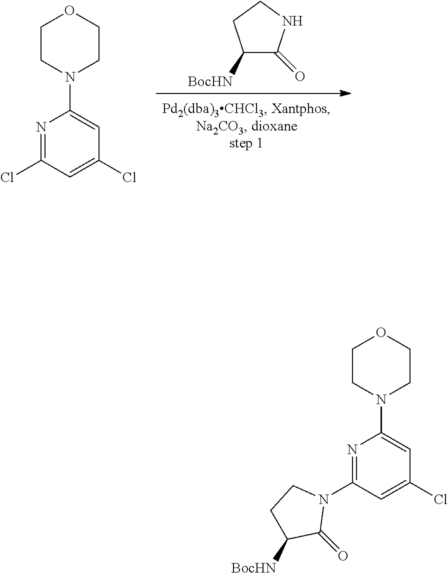 [SMILES: Clc1cc(Cl)nc(N2CCOCC2)c1 ; CC(C)(C)OC(=O)N[C@H]1CCNC1=O ; CC(C)(C)OC(=O)N[C@H]1CCN(c2cc(Cl)cc(N3CCOCC3)n2)C1=O]

A mixture of 4-(4,6-dichloropyridin-2-yl)morpholine (500 mg, 2.145 mmol), tert-butyl N-[(3S)-2-oxopyrrolidin-3-yl]carbamate (644 mg, 3.218 mmol), dioxane (5 mL), Pd₂(dba)₃·CHCl₃ (222 mg, 0.215 mmol), XantPhos (248 mg, 0.429 mmol) and Na₂CO₃ (682 mg, 6.435 mmol) was stirred for 2 h at 100° C. under nitrogen atmosphere. The resulting mixture was quenched with water (50 mL). The resulting mixture was extracted with EtOAc (3×50 mL). The combined organic layers were washed with brine (50 mL), dried over anhydrous Na₂SO₄. After filtration, the filtrate was concentrated under reduced pressure. The residue was purified by silica gel column chromatography, eluted with PE/EtOAc (1/1) to afford tert-butyl N-[(3S)-1-[4-chloro-6-(morpholin-4-yl)pyridin-2-yl]-2-oxopyrrolidin-3-yl]carbamate (740 mg, 86%) as a yellow solid. MS ESI calculated for C₁₈H₂₅ClN₄O₄[M+H]⁺, 397.16, found 397.25. ¹H NMR (400 MHz, Chloroform-d) δ 7.80 (d, J=1.2 Hz, 1H), 6.38 (d, J=1.2 Hz, 1H), 5.22 (s, 1H), 4.43-4.40 (m, 1H), 4.26-4.16 (m, 1H), 3.85-3.78 (m, 4H), 3.75-3.71 (m, 1H), 3.55-3.42 (m, 4H), 2.74-2.70 (m, 1H), 1.93-1.90 (m, 1H), 1.49 (s, 9H).

Preparation 121B: tert-butyl N-[(3S)-1-(4-[2-methyl-5-[(3S)-3-(2,2,2-trifluoroethyl)pyrrolidine-1-carbonylamino]phenyl]-6-(morpholin-4-yl)pyridin-2-yl)-2-oxopyrrolidin-3-yl]carbamate

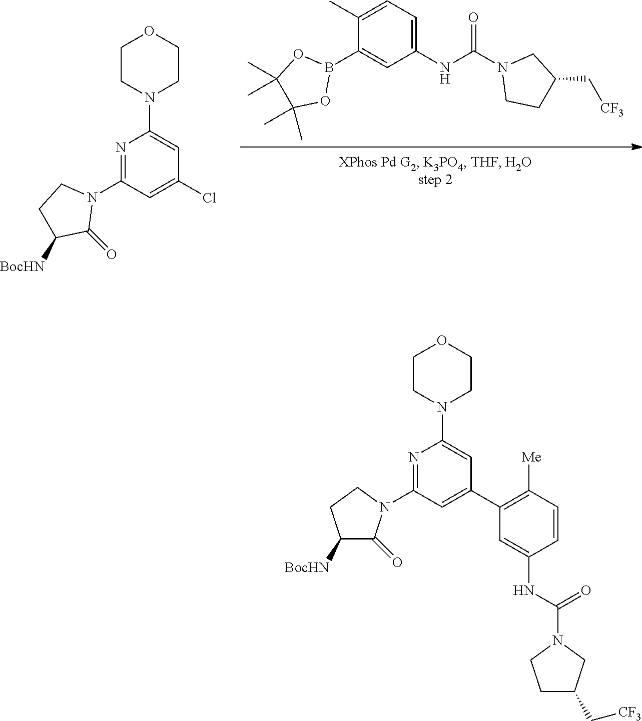 [SMILES: CC(C)(C)OC(=O)N[C@H]1CCN(c2cc(Cl)cc(N3CCOCC3)n2)C1=O ; Cc1ccc(NC(=O)N2CC[C@@H](CC(F)(F)F)C2)cc1B1OC(C)(C)C(C)(C)O1 ; Cc1ccc(NC(=O)N2CC[C@@H](CC(F)(F)F)C2)cc1-c1cc(N2CCOCC2)nc(N2CC[C@H](NC(=O)OC(C)(C)C)C2=O)c1]

A mixture of tert-butyl N-[(3S)-1-[4-chloro-6-(morpholin-4-yl)pyridin-2-yl]-2-oxopyrrolidin-3-yl]carbamate (200 mg, 0.504 mmol), (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (228 mg, 0.554 mmol), THF (5 mL), H₂O (0.5 mL), 2^{nd} XPhos Precatalyst (39 mg, 0.050 mmol) and K₃PO₄ (213 mg, 1.008 mmol) was stirred for 2 h at 80° C. under a nitrogen atmosphere. The resulting mixture was concentrated under reduced pressure. The residue was purified by Prep-TLC with PE/EtOAc/EtOH (4/3/1) to afford tert-butyl N-[(3S)-1-(4-[2-methyl-5-[(3S)-3-(2,2,2-trifluoroethyl)pyrrolidine-1-carbonylamino]phenyl]-6-(morpholin-4-yl)pyridin-2-yl)-2-oxopyrrolidin-3-yl]carbamate (300 mg, 92%) as a yellow oil. MS ESI calculated for C₃₂H₄₁F₃N₆O₅[M+H]⁺, 647.31, found 647.35.

### Example 121: (3S)-N-(3-[2-[(3S)-3-amino-2-oxopyrrolidin-1-yl]-6-(morpholin-4-yl)pyridin-4-yl]-4-methylphenyl)-3-(2,2,2-trifluoroethyl)pyrrolidine-1-carboxamide

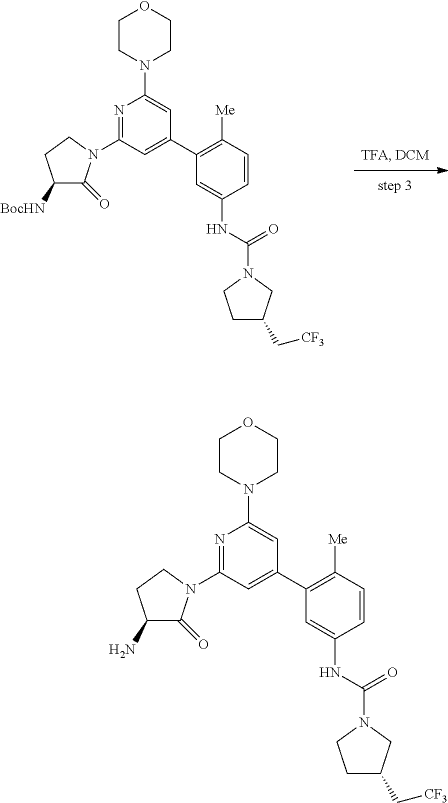 [SMILES: Cc1ccc(NC(=O)N2CC[C@@H](CC(F)(F)F)C2)cc1-c1cc(N2CCOCC2)nc(N2CC[C@H](NC(=O)OC(C)(C)C)C2=O)c1 ; Cc1ccc(NC(=O)N2CC[C@@H](CC(F)(F)F)C2)cc1-c1cc(N2CCOCC2)nc(N2CC[C@H](N)C2=O)c1]

A solution of tert-butyl N-[(3S)-1-(4-[2-methyl-5-[(3S)-3-(2,2,2-trifluoroethyl)pyrrolidine-1-carbonylamino]phenyl]-6-(morpholin-4-yl)pyridin-2-yl)-2-oxopyrrolidin-3-yl]carbamate (200 mg, 0.309 mmol) and TFA (1 mL) in DCM (5 mL) was stirred for 1 h at room temperature. The resulting mixture was concentrated under reduced pressure. The crude product was purified by reverse phase flash with the following conditions: Column: Spherical C18, 20-40 μm, 120 g; Mobile Phase A: water (plus 0.05% FA); Mobile Phase B: CH₃CN; Flow rate: 60 mL/min; Gradient: 35% B to 75%, Detector: 220 nm. The resulting mixture was diluted with Na₂CO₃ (1 M, 10 mL). The resulting mixture was extracted with EtOAc (2×15 mL). The combined organic layers were washed with brine (15 mL), dried over anhydrous Na₂SO₄. After filtration, the filtrate was concentrated under reduced pressure to afford (3S)-N-(3-[2-[(3S)-3-amino-2-oxopyrrolidin-1-yl]-6-(morpholin-4-yl)pyridin-4-yl]-4-methylphenyl)-3-(2,2,2-trifluoroethyl)pyrrolidine-1-carboxamide (129 mg, 76%) as an off-white solid. MS ESI calculated for C₂₇H₃₃F₃N₆O₃ [M+H]⁺, 547.26, found 547.25. ¹H NMR (400 MHz, DMSO-d₆) δ 8.19 (s, 1H), 7.62 (s, 1H), 7.47 (dd, J=2.0, 8.4 Hz, 1H), 7.39 (d, J=2.0 Hz, 1H), 7.15 (d, J=8.4 Hz, 1H), 6.45 (s, 1H), 4.13-4.04 (m, 1H), 3.77-3.65 (m, 6H), 3.61-3.46 (m, 6H), 3.31-3.27 (m, 1H), 3.05-3.00 (m, 1H), 2.51-2.40 (m, 3H), 2.35-2.31 (m, 1H), 2.17 (s, 3H), 2.12-2.05 (m, 1H), 1.91 (s, 2H), 1.78-1.56 (m, 2H). ¹⁹F NMR (376 MHz, DMSO-d₆) δ-63.37 (3F).

### Example 122: (3S)-N-(3-[2-[(4S)-4-amino-2-oxopyrrolidin-1-yl]-6-(morpholin-4-yl)pyridin-4-yl]-4-methylphenyl)-3-(2,2,2-trifluoroethyl)pyrrolidine-1-carboxamide

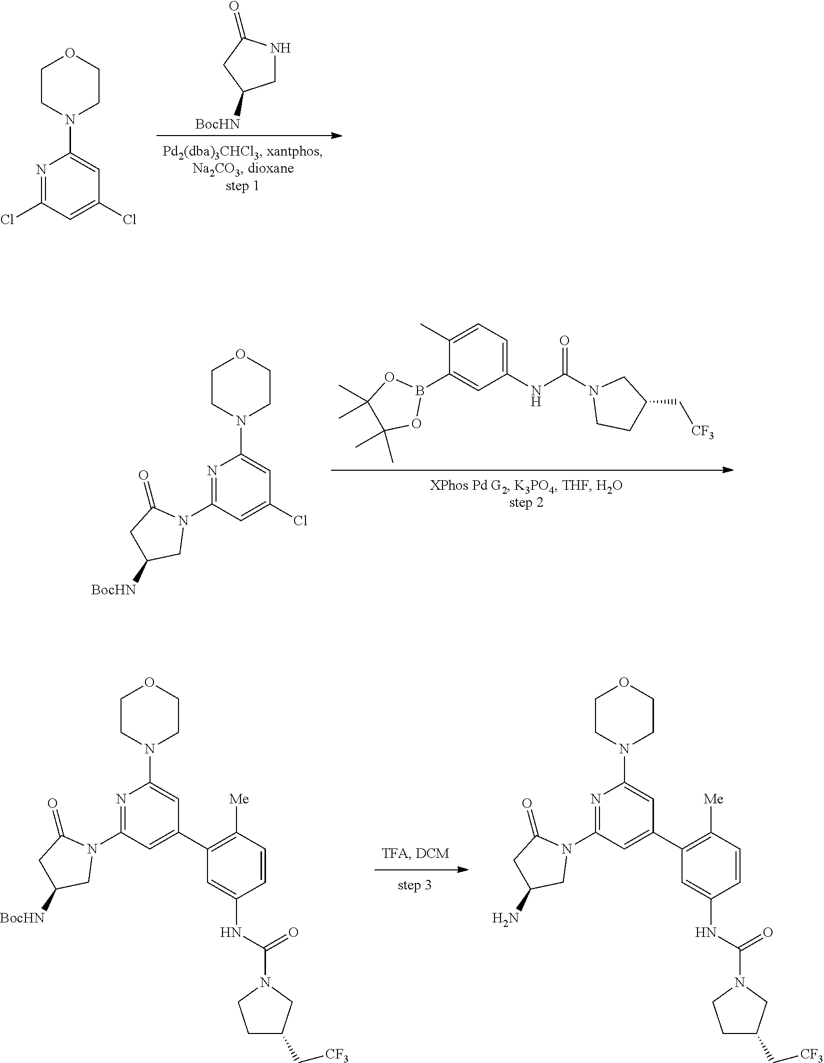 [SMILES: Clc1cc(Cl)nc(N2CCOCC2)c1 ; CC(C)(C)OC(=O)N[C@@H]1CNC(=O)C1 ; CC(C)(C)OC(=O)N[C@H]1CC(=O)N(c2cc(Cl)cc(N3CCOCC3)n2)C1 ; Cc1ccc(NC(=O)N2CC[C@@H](CC(F)(F)F)C2)cc1B1OC(C)(C)C(C)(C)O1 ; Cc1ccc(NC(=O)N2CC[C@@H](CC(F)(F)F)C2)cc1-c1cc(N2CCOCC2)nc(N2C[C@@H](NC(=O)OC(C)(C)C)CC2=O)c1 ; Cc1ccc(NC(=O)N2CC[C@@H](CC(F)(F)F)C2)cc1-c1cc(N2CCOCC2)nc(N2C[C@@H](N)CC2=O)c1]

Preparation 122A: tert-butyl N-[(3S)-1-[4-chloro-6-(morpholin-4-yl)pyridin-2-yl]-5-oxopyrrolidin-3-yl]carbamate

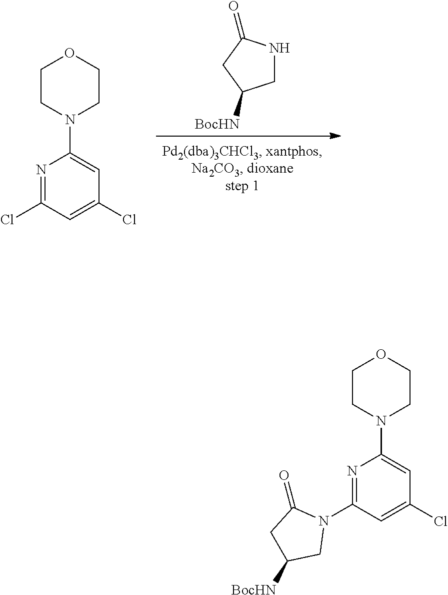 [SMILES: Clc1cc(Cl)nc(N2CCOCC2)c1 ; CC(C)(C)OC(=O)N[C@@H]1CNC(=O)C1 ; CC(C)(C)OC(=O)N[C@H]1CC(=O)N(c2cc(Cl)cc(N3CCOCC3)n2)C1]

A mixture of 4-(4,6-dichloropyridin-2-yl)morpholine (500 mg, 2.145 mmol), tert-butyl N-[(3S)-5-oxopyrrolidin-3-yl]carbamate (644 mg, 3.218 mmol), Pd₂(dba)₃·CHCl₃ (222 mg, 0.215 mmol), Na₂CO₃ (682 mg, 6.435 mmol) and XantPhos (248 mg, 0.429 mmol) in dioxane (5 mL) was stirred for 2 h at 100° C. under nitrogen atmosphere. The resulting mixture was diluted with water (30 mL). The resulting mixture was extracted with EtOAc (3×30 mL). The combined organic layers were washed with brine (30 mL), dried over anhydrous Na₂SO₄. After filtration, the filtrate was concentrated under reduced pressure. The residue was purified by silica gel column chromatography, eluted with PE/EtOAc (1/1) to afford tert-butyl N-[(3S)-1-[4-chloro-6-(morpholin-4-yl)pyridin-2-yl]-5-oxopyrrolidin-3-yl]carbamate (750 mg, 88%) as a light yellow solid. MS ESI calculated for C₁₈H₂₅ClN₄O₄[M+H]⁺, 397.16, found 397.20. ¹H NMR (400 MHz, Chloroform-d) δ 7.80 (d, J=1.2 Hz, 1H), 6.40 (d, J=1.2 Hz, 1H), 4.85-4.83 (m, 1H), 4.40-4.38 (m, 1H), 4.31-4.27 (m, 1H), 4.01-3.93 (m, 1H), 3.86-3.79 (m, 4H), 3.54-3.45 (m, 4H), 3.04-3.01 (m, 1H), 2.58-2.54 (m, 1H), 1.47 (s, 9H).

Preparation 122B: tert-butyl N-[(3S)-1-(4-[2-methyl-5-[(3S)-3-(2,2,2-trifluoroethyl)pyrrolidine-1-carbonylamino]phenyl]-6-(morpholin-4-yl)pyridin-2-yl)-5-oxopyrrolidin-3-yl]carbamate

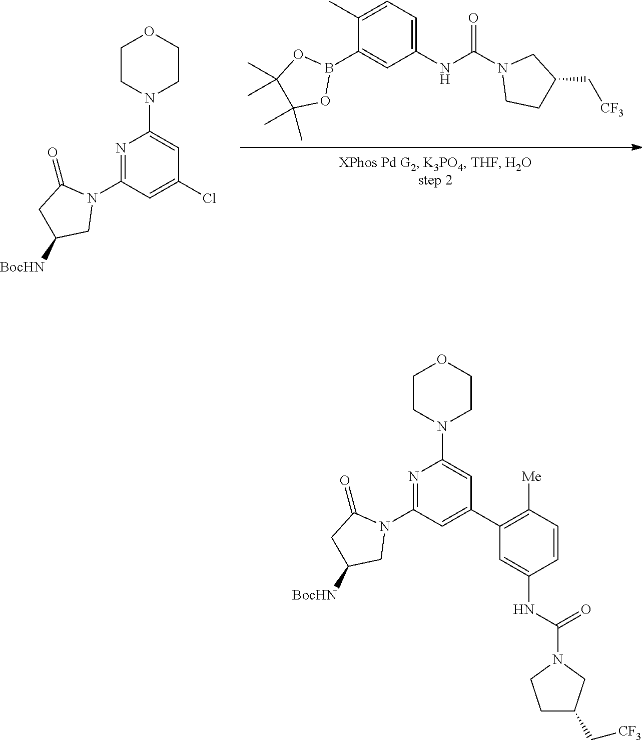 [SMILES: CC(C)(C)OC(=O)N[C@H]1CC(=O)N(c2cc(Cl)cc(N3CCOCC3)n2)C1 ; Cc1ccc(NC(=O)N2CC[C@@H](CC(F)(F)F)C2)cc1B1OC(C)(C)C(C)(C)O1 ; Cc1ccc(NC(=O)N2CC[C@@H](CC(F)(F)F)C2)cc1-c1cc(N2CCOCC2)nc(N2C[C@@H](NC(=O)OC(C)(C)C)CC2=O)c1]

To a stirred mixture of tert-butyl N-[(3S)-1-[4-chloro-6-(morpholin-4-yl)pyridin-2-yl]-5-oxopyrrolidin-3-yl]carbamate (200 mg, 0.504 mmol) and (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (249 mg, 0.605 mmol) in THF (6 mL) and H₂O (0.6 mL) were added 2^{nd} XPhos Precatalyst (39 mg, 0.050 mmol) and K₃PO₄ (213 mg, 1.008 mmol. The resulting mixture was stirred for 2 h at 80° C. under nitrogen atmosphere. The resulting mixture was concentrated under reduced pressure. The residue was purified by silica gel column chromatography, eluted with PE/EtOAc/EtOH (4/3/1) to afford tert-butyl N-[(3S)-1-(4-[2-methyl-5-[(3S)-3-(2,2,2-trifluoroethyl)pyrrolidine-1-carbonylamino]phenyl]-6-(morpholin-4-yl)pyridin-2-yl)-5-oxopyrrolidin-3-yl]carbamate (300 mg, 92%) as a light yellow solid. MS ESI calculated for C₃₂H₄₁F₃N₆O₅[M+H]⁺, 647.31, found 647.15.

### Example 122: (3S)-N-(3-[2-[(4S)-4-amino-2-oxopyrrolidin-1-yl]-6-(morpholin-4-yl)pyridin-4-yl]-4-methylphenyl)-3-(2,2,2-trifluoroethyl)pyrrolidine-1-carboxamide

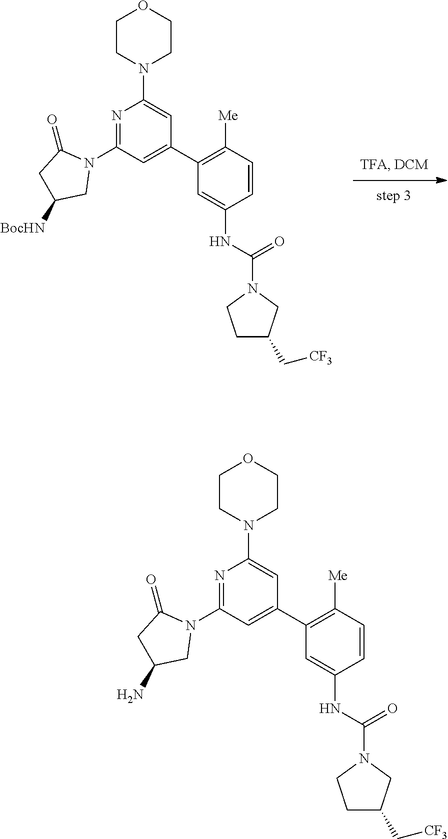 [SMILES: Cc1ccc(NC(=O)N2CC[C@@H](CC(F)(F)F)C2)cc1-c1cc(N2CCOCC2)nc(N2C[C@@H](NC(=O)OC(C)(C)C)CC2=O)c1 ; Cc1ccc(NC(=O)N2CC[C@@H](CC(F)(F)F)C2)cc1-c1cc(N2CCOCC2)nc(N2C[C@@H](N)CC2=O)c1]

To a stirred solution of tert-butyl N-[(3S)-1-(4-[2-methyl-5-[(3S)-3-(2,2,2-trifluoroethyl)pyrrolidine-1-carbonylamino]phenyl]-6-(morpholin-4-yl)pyridin-2-yl)-5-oxopyrrolidin-3-yl]carbamate (200 mg, 0.309 mmol) in DCM (4 mL) was added TFA (0.8 mL) at room temperature. The resulting mixture was stirred for 1 h at room temperature. The resulting mixture was concentrated under reduced pressure. The crude product was purified by reverse phase flash with the following conditions: Column: Spherical C18, 20-40 μm, 120 g; Mobile Phase A: Water (plus 0.05% FA); Mobile Phase B: CH₃CN; Flow rate: 60 mL/min; Gradient: 25% B to 55% B, Detector: 220 nm to afford (3S)-N-(3-[2-[(4S)-4-amino-2-oxopyrrolidin-1-yl]-6-(morpholin-4-yl)pyridin-4-yl]-4-methylphenyl)-3-(2,2,2-trifluoroethyl)pyrrolidine-1-carboxamide (66 mg, 39%) as an off-white solid. MS ESI calculated for C₂₇H₃₃F₃N₆O₃ [M+H]⁺, 547.26, found 547.30. ¹H NMR (400 MHz, DMSO-d₆) δ 8.17 (s, 1H), 7.59 (s, 1H), 7.47 (dd, J=2.4, 8.4 Hz, 1H), 7.38 (d, J=2.4 Hz, 1H), 7.14 (d, J=8.8 Hz, 1H), 6.43 (s, 1H), 4.08-4.04 (m, 1H), 3.76-3.59 (m, 6H), 3.63-3.53 (m, 1H), 3.56-3.43 (m, 5H), 3.03 (t, J=9.6 Hz, 1H), 2.78-2.74 (m, 1H), 2.48-2.36 (m, 3H), 2.25-2.22 (m, 1H), 2.16 (s, 3H), 2.10-2.06 (m, 1H), 1.85 (brs, 2H), 1.66-1.55 (m, 1H). ¹⁹F NMR (282 MHz, Chloroform-d) δ-64.94 (3F).

### Example 123: (3S)-N-(3-[2-[(3S)-3-amino-3-methylpyrrolidin-1-yl]-6-(morpholin-4-yl)pyridin-4-yl]-4-methylphenyl)-3-(2,2,2-trifluoroethyl)pyrrolidine-1-carboxamide

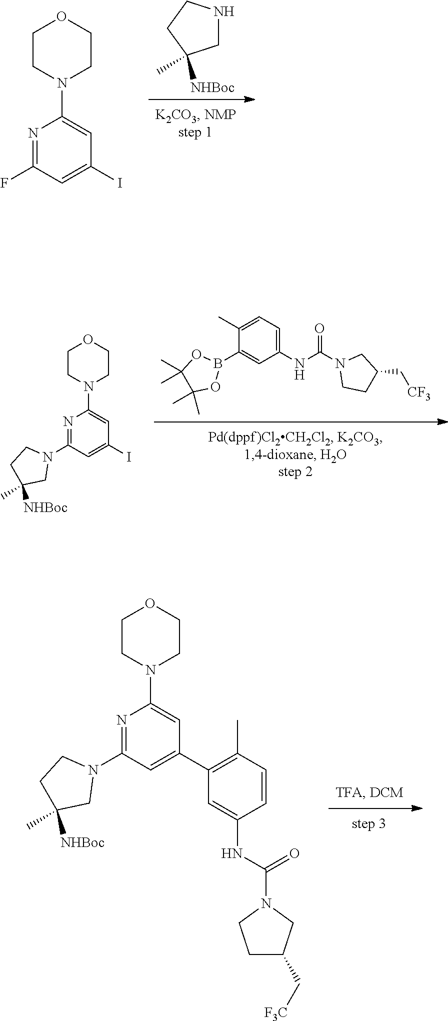 [SMILES: Fc1cc(I)cc(N2CCOCC2)n1 ; CC(C)(C)OC(=O)N[C@@]1(C)CCNC1 ; CC(C)(C)OC(=O)N[C@@]1(C)CCN(c2cc(I)cc(N3CCOCC3)n2)C1 ; Cc1ccc(NC(=O)N2CC[C@@H](CC(F)(F)F)C2)cc1B1OC(C)(C)C(C)(C)O1 ; Cc1ccc(NC(=O)N2CC[C@@H](CC(F)(F)F)C2)cc1-c1cc(N2CCOCC2)nc(N2CC[C@](C)(NC(=O)OC(C)(C)C)C2)c1]

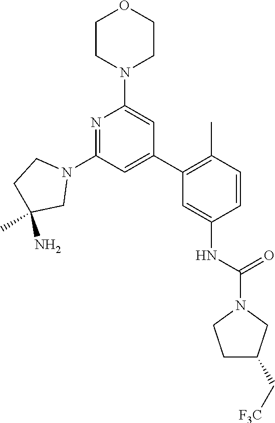 [SMILES: Cc1ccc(NC(=O)N2CC[C@@H](CC(F)(F)F)C2)cc1-c1cc(N2CCOCC2)nc(N2CC[C@](C)(N)C2)c1]

Preparation 123A: tert-butyl N-[(3S)-1-[4-iodo-6-(morpholin-4-yl)pyridin-2-yl]-3-methylpyrrolidin-3-yl]carbamate

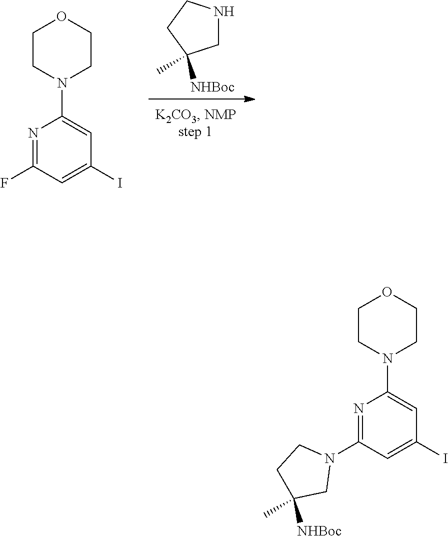 [SMILES: Fc1cc(I)cc(N2CCOCC2)n1 ; CC(C)(C)OC(=O)N[C@@]1(C)CCNC1 ; CC(C)(C)OC(=O)N[C@@]1(C)CCN(c2cc(I)cc(N3CCOCC3)n2)C1]

To a stirred solution of 4-(6-fluoro-4-iodopyridin-2-yl)morpholine (693 mg, 2.249 mmol) and tert-butyl N-[(3S)-3-methylpyrrolidin-3-yl]carbamate (450 mg, 2.249 mmol) in NMP (15 mL) was added K₂CO₃ (932 mg, 6.748 mmol). The resulting mixture was stirred for 2 h at 150° C. under nitrogen atmosphere. The resulting mixture was diluted with EtOAc (50 mL). The combined organic layers were washed with brine (3×20 mL), dried over anhydrous Na₂SO₄. After filtration, the filtrate was concentrated under reduced pressure. The residue was purified by silica gel column chromatography, eluted with PE/EtOAc (3/1) to afford tert-butyl N-[(3S)-1-[4-iodo-6-(morpholin-4-yl)pyridin-2-yl]-3-methylpyrrolidin-3-yl]carbamate (700 mg, 63%) as a white solid. MS ESI calculated for C₁₉H₂₉IN₄O₃[M+H]⁺, 489.13, found 489.05. ¹H NMR (400 MHz, Chloroform-d) δ 6.26 (d, J=0.8 Hz, 1H), 6.15 (d, J=0.8 Hz, 1H), 4.66 (s, 1H), 3.85-3.76 (m, 4H), 3.64 (d, J=10.8 Hz, 1H), 3.54-3.43 (m, 6H), 3.39 (d, J=10.8 Hz, 1H), 2.43-2.33 (m, 1H), 1.95-1.88 (m, 1H), 1.51 (s, 3H), 1.45 (s, 9H).

Preparation 123B: tert-butyl N-[(3S)-3-methyl-1-(4-[2-methyl-5-[(3S)-3-(2,2,2-trifluoroethyl)pyrrolidine-1-carbonylamino]phenyl]-6-(morpholin-4-yl)pyridin-2-yl)pyrrolidin-3-yl]carbamate

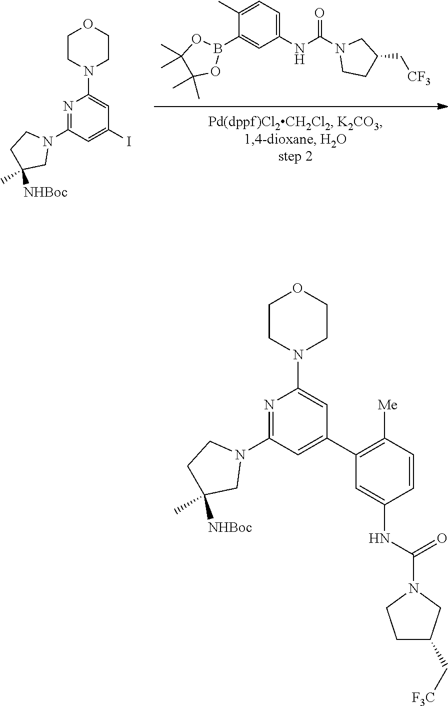 [SMILES: CC(C)(C)OC(=O)N[C@@]1(C)CCN(c2cc(I)cc(N3CCOCC3)n2)C1 ; Cc1ccc(NC(=O)N2CC[C@@H](CC(F)(F)F)C2)cc1B1OC(C)(C)C(C)(C)O1 ; Cc1ccc(NC(=O)N2CC[C@@H](CC(F)(F)F)C2)cc1-c1cc(N2CCOCC2)nc(N2CC[C@](C)(NC(=O)OC(C)(C)C)C2)c1]

To a stirred solution of tert-butyl N-[(3S)-1-[4-iodo-6-(morpholin-4-yl)pyridin-2-yl]-3-methylpyrrolidin-3-yl]carbamate (200 mg, 0.410 mmol) and (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (185 mg, 0.450 mmo) in dioxane (6 mL) and H₂O (1.5 mL) were added Pd(dppf)Cl₂·CH₂Cl₂ (33 mg, 0.041 mmol) and Na₂CO₃ (130 mg, 1.230 mmol) at room temperature. The resulting mixture was stirred for 2 h at 60° C. under nitrogen atmosphere. The residue was purified by Prep-TLC (PE/EtOAc=1/1) to afford tert-butyl N-[(3S)-3-methyl-1-(4-[2-methyl-5-[(3S)-3-(2,2,2-trifluoroethyl)pyrrolidine-1-carbonylamino]phenyl]-6-(morpholin-4-yl)pyridin-2-yl)pyrrolidin-3-yl]carbamate (220 mg, 83%) as a light brown solid. MS ESI calculated for C₃₃H₄₅F₃N₆O₄[M+H]⁺, 647.35, found 647.30.

### Example 123: (3S)-N-(3-[2-[(3S)-3-amino-3-methylpyrrolidin-1-yl]-6-(morpholin-4-yl)pyridin-4-yl]-4-methylphenyl)-3-(2,2,2-trifluoroethyl)pyrrolidine-1-carboxamide

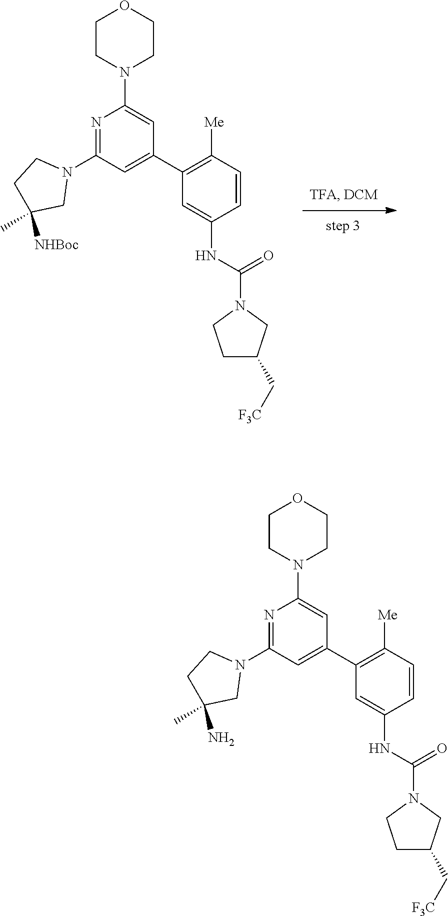 [SMILES: Cc1ccc(NC(=O)N2CC[C@@H](CC(F)(F)F)C2)cc1-c1cc(N2CCOCC2)nc(N2CC[C@](C)(NC(=O)OC(C)(C)C)C2)c1 ; Cc1ccc(NC(=O)N2CC[C@@H](CC(F)(F)F)C2)cc1-c1cc(N2CCOCC2)nc(N2CC[C@](C)(N)C2)c1]

A solution of tert-butyl N-[(3S)-3-methyl-1-(4-[2-methyl-5-[(3S)-3-(2,2,2-trifluoroethyl)pyrrolidine-1-carbonylamino]phenyl]-6-(morpholin-4-yl)pyridin-2-yl)pyrrolidin-3-yl]carbamate (200 mg, 0.309 mmol) and TFA (1 mL) in DCM (5 mL) was stirred for 1 h at room temperature. The resulting mixture was concentrated under reduced pressure. The resulting mixture was diluted with EtOAc (30 mL). The combined organic layers were washed with brine (10 mL), dried over anhydrous Na₂SO₄. After filtration, the filtrate was concentrated under reduced pressure. The crude product was purified by reverse phase flash with the following conditions: Column: Spherical C18, 20-40 μm, 120 g; Mobile Phase A: Water (plus 10 mM NH₄CO₃); Mobile Phase B: CH₃CN; Flow rate: 60 mL/min; Gradient: 25% B to 55% B, Detector: 220 nm to afford (3S)-N-(3-[2-[(3S)-3-amino-3-methylpyrrolidin-1-yl]-6-(morpholin-4-yl)pyridin-4-yl]-4-methylphenyl)-3-(2,2,2-trifluoroethyl)pyrrolidine-1-carboxamide (88 mg, 52%) as a light green solid. MS ESI calculated for C₂₈H₃₇F₃N₆O₂ [M+H]⁺, 547.29, found 547.30. ¹H NMR (300 MHz, DMSO-d₆) δ 8.13 (s, 1H), 7.44 (dd, J=2.4, 8.4 Hz, 1H), 7.35 (d, J=2.4 Hz, 1H), 7.11 (d, J=8.4 Hz, 1H), 5.84 (s, 1H), 5.61 (s, 1H), 3.72-3.68 (m, 5H), 3.55-3.23 (m, 10H), 3.04 (t, J=9.3 Hz, 1H), 2.49-2.37 (m, 3H), 2.18 (s, 3H), 2.11-2.07 (m, 1H), 1.81-1.60 (m, 5H), 1.25 (s, 3H). ¹⁹F NMR (282 MHz, DMSO-d₆) δ-63.35 (3F).

### Example 124: (3S)-N-(3-[2-[(3S)-3-amino-3-methylpiperidin-1-yl]-6-(morpholin-4-yl)pyridin-4-yl]-4-methylphenyl)-3-(2,2,2-trifluoroethyl)pyrrolidine-1-carboxamide

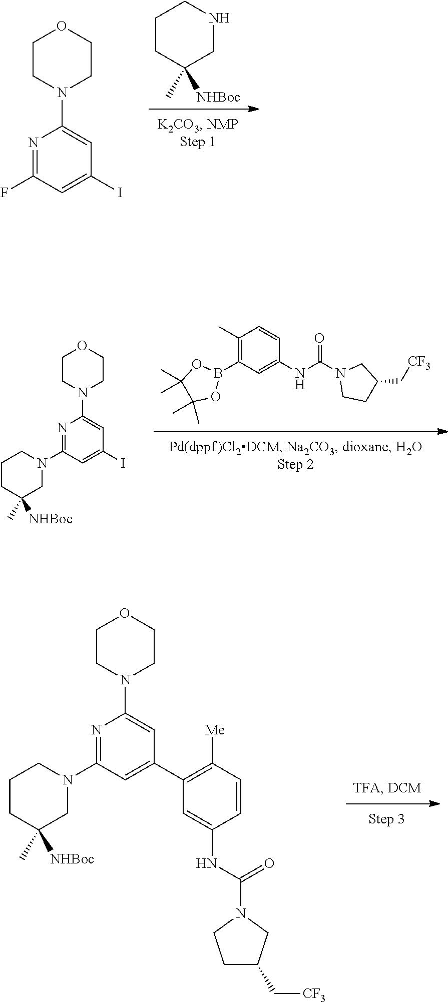 [SMILES: Fc1cc(I)cc(N2CCOCC2)n1 ; CC(C)(C)OC(=O)N[C@@]1(C)CCCNC1 ; CC(C)(C)OC(=O)N[C@@]1(C)CCCN(c2cc(I)cc(N3CCOCC3)n2)C1 ; Cc1ccc(NC(=O)N2CC[C@@H](CC(F)(F)F)C2)cc1B1OC(C)(C)C(C)(C)O1 ; Cc1ccc(NC(=O)N2CC[C@@H](CC(F)(F)F)C2)cc1-c1cc(N2CCOCC2)nc(N2CCC[C@](C)(NC(=O)OC(C)(C)C)C2)c1]

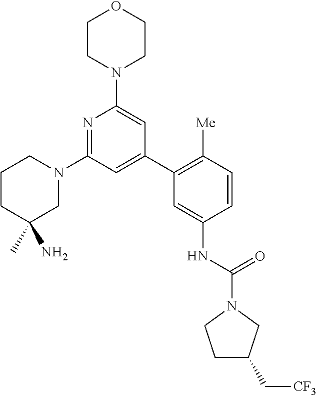 [SMILES: Cc1ccc(NC(=O)N2CC[C@@H](CC(F)(F)F)C2)cc1-c1cc(N2CCOCC2)nc(N2CCC[C@](C)(N)C2)c1]

Preparation 124A: tert-butyl N-[(3S)-1-[4-iodo-6-(morpholin-4-yl)pyridin-2-yl]-3-methylpiperidin-3-yl]carbamate

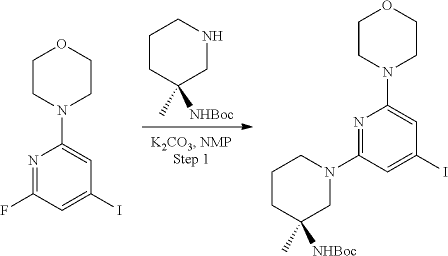 [SMILES: Fc1cc(I)cc(N2CCOCC2)n1 ; CC(C)(C)OC(=O)N[C@@]1(C)CCCNC1 ; CC(C)(C)OC(=O)N[C@@]1(C)CCCN(c2cc(I)cc(N3CCOCC3)n2)C1]

To a stirred solution of 4-(6-fluoro-4-iodopyridin-2-yl)morpholine (646 mg, 2.100 mmol) and tert-butyl N-[(3S)-3-methylpiperidin-3-yl]carbamate (450 mg, 2.100 mmol) in NMP (5 mL) was added K₂CO₃ (870 mg, 6.299 mmol). The resulting mixture was stirred for 2 h at 150° C. under nitrogen atmosphere. The resulting mixture was diluted with EtOAc (30 mL). The combined organic layers were washed with brine (10 mL), dried over anhydrous Na₂SO₄. After filtration, the filtrate was concentrated under reduced pressure. The residue was purified by silica gel column chromatography, eluted with PE/EtOAc/EtOH (12/3/1) to afford tert-butyl N-[(3S)-1-[4-iodo-6-(morpholin-4-yl)pyridin-2-yl]-3-methylpiperidin-3-yl]carbamate (270 mg, 25%) as a light yellow solid. MS ESI calculated for C₂₀H₃₁IN₄O₃[M+H]⁺, 503.14, found 503.00. ¹H NMR (400 MHz, Chloroform-d) δ 6.46 (s, 1H), 6.31 (s, 1H), 5.07 (s, 1H), 4.20 (d, J=13.2 Hz, 1H), 3.97 (d, J=12.9 Hz, 1H), 3.83-3.79 (m, 4H), 3.48-3.42 (m, 4H), 3.03-2.82 (m, 1H), 2.77 (d, J=13.2 Hz, 1H), 2.40-2.32 (m, 1H), 1.75-1.55 (m, 3H), 1.43 (s, 9H), 1.39 (s, 3H).

Preparation 124B: tert-butyl N-[(3S)-3-methyl-1-(4-[2-methyl-5-[(3S)-3-(2,2,2-trifluoroethyl)pyrrolidine-1-carbonylamino]phenyl]-6-(morpholin-4-yl)pyridin-2-yl)piperidin-3-yl]carbamate

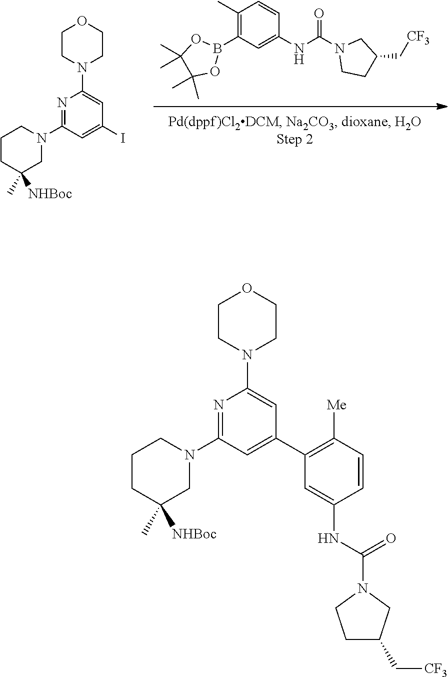 [SMILES: CC(C)(C)OC(=O)N[C@@]1(C)CCCN(c2cc(I)cc(N3CCOCC3)n2)C1 ; Cc1ccc(NC(=O)N2CC[C@@H](CC(F)(F)F)C2)cc1B1OC(C)(C)C(C)(C)O1 ; Cc1ccc(NC(=O)N2CC[C@@H](CC(F)(F)F)C2)cc1-c1cc(N2CCOCC2)nc(N2CCC[C@](C)(NC(=O)OC(C)(C)C)C2)c1]

To a stirred solution of tert-butyl N-[(3S)-1-[4-iodo-6-(morpholin-4-yl)pyridin-2-yl]-3-methylpiperidin-3-yl]carbamate (200 mg, 0.398 mmol) and (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (180 mg, 0.438 mmol) in 1,4-dioxane (2 mL) and H₂O (0.5 mL) were added Pd(dppf)Cl₂·CH₂Cl₂ (32 mg, 0.040 mmol) and Na₂CO₃ (126.58 mg, 1.194 mmol). The resulting mixture was stirred for 2 h at 60° C. under nitrogen atmosphere. The resulting mixture was concentrated under reduced pressure. The residue was purified by Prep-TLC with PE/EtOAc (1/1) to afford tert-butyl N-[(3S)-3-methyl-1-(4-[2-methyl-5-[(3S)-3-(2,2,2-trifluoroethyl)pyrrolidine-1-carbonylamino]phenyl]-6-(morpholin-4-yl)pyridin-2-yl)piperidin-3-yl]carbamate (230 mg, 87%) as a light brown solid. MS ESI calculated for C₃₄H₄₇F₃N₆O₄[M+H]⁺, 661.36, found 661.40.

### Example 124: (3S)-N-(3-[2-[(3S)-3-amino-3-methylpiperidin-1-yl]-6-(morpholin-4-yl)pyridin-4-yl]-4-methylphenyl)-3-(2,2,2-trifluoroethyl)pyrrolidine-1-carboxamide

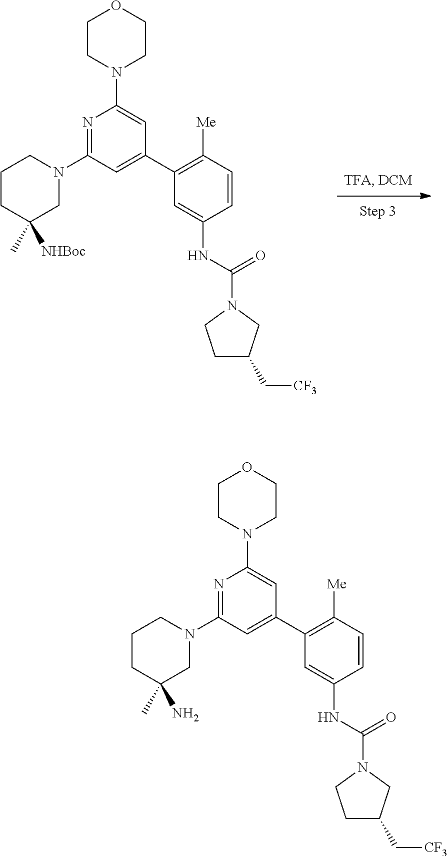 [SMILES: Cc1ccc(NC(=O)N2CC[C@@H](CC(F)(F)F)C2)cc1-c1cc(N2CCOCC2)nc(N2CCC[C@](C)(NC(=O)OC(C)(C)C)C2)c1 ; Cc1ccc(NC(=O)N2CC[C@@H](CC(F)(F)F)C2)cc1-c1cc(N2CCOCC2)nc(N2CCC[C@](C)(N)C2)c1]

A solution of tert-butyl N-[(3S)-3-methyl-1-(4-[2-methyl-5-[(3S)-3-(2,2,2-trifluoroethyl)pyrrolidine-1-carbonylamino]phenyl]-6-(morpholin-4-yl)pyridin-2-yl)piperidin-3-yl]carbamate (200 mg, 0.303 mmol) and TFA (0.4 mL) in DCM (2 mL) was stirred for 1 h at room temperature. The resulting mixture was concentrated under reduced pressure. The residue was purified by silica gel column chromatography, eluted with CH₂Cl₂/MeOH (8/1). The crude product was purified by Prep-HPLC with the following conditions: Column: XBridge Prep Phenyl OBD Column, 19×150 mm, 5 μm; Mobile Phase A: water (10 mmol/L NH₄HCO₃), Mobile Phase B: CH₃CN; Flow rate:20 mL/min; Gradient: 40% B to 50% B; 254/210 nm to afford (3S)-N-(3-[2-[(3S)-3-amino-3-methylpiperidin-1-yl]-6-(morpholin-4-yl)pyridin-4-yl]-4-methylphenyl)-3-(2,2,2-trifluoroethyl)pyrrolidine-1-carboxamide (29 mg, 17%) as a white solid. MS ESI calculated for C₂₉H₃₉F₃N₆O₂[M+H]⁺, 560.31, found 561.30. ¹H NMR (400 MHz, DMSO-d₆) δ 8.13 (s, 1H), 7.43 (dd, J=8.4, 2.4 Hz, 1H), 7.34 (d, J=2.4 Hz, 1H), 7.11 (d, J=8.4 Hz, 1H), 6.01 (s, 1H), 5.91 (s, 1H), 3.73-3.62 (m, 5H), 3.54-3.52 (m, 2H), 3.42-3.38 (m, 5H), 3.30-3.25 (m, 3H), 3.02 (t, J=9.6 Hz, 1H), 2.50-2.42 (m, 4H), 2.16 (s, 3H), 2.10-2.06 (m, 1H), 1.67-1.60 (m, 2H), 1.55-1.50 (m, 3H), 1.07 (s, 3H). ¹⁹F NMR (376 MHz, DMSO-d₆) δ-63.37 (3F).

### Example 125: (3S)-N-(3-[2-[(4R)-4-hydroxy-2-oxopyrrolidin-1-yl]-6-(morpholin-4-yl)pyridin-4-yl]-4-methylphenyl)-3-(2,2,2-trifluoroethyl)pyrrolidine-1-carboxamide

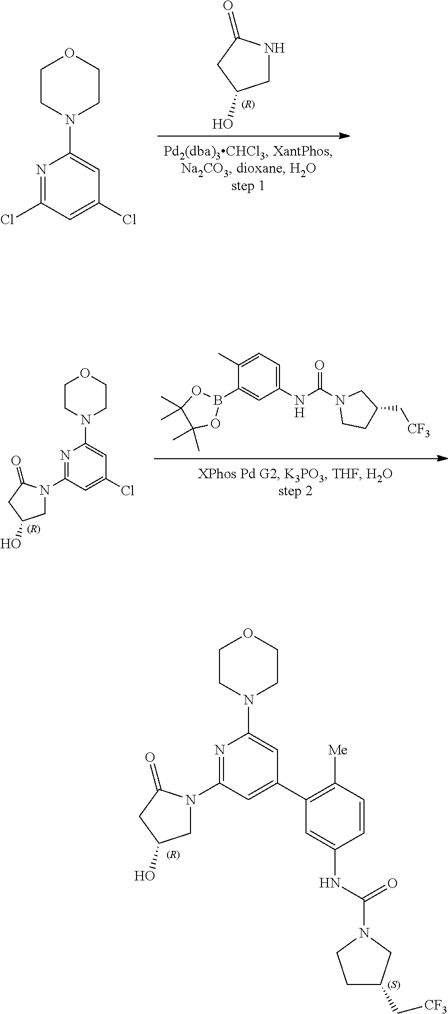

Preparation 125A: (4R)-1-[4-chloro-6-(morpholin-4-yl)pyridin-2-yl]-4-hydroxypyrrolidin-2-one

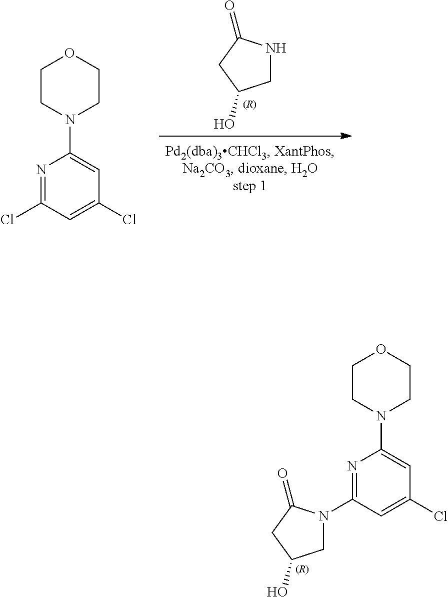

A mixture of 4-(4,6-dichloropyridin-2-yl)morpholine (500 mg, 2.145 mmol), (4R)-4-hydroxypyrrolidin-2-one (195 mg, 1.931 mmol), Pd₂(dba)₃-CHCl₃ (222 mg, 0.215 mmol), XantPhos (248 mg, 0.429 mmol) and Na₂CO₃ (455 mg, 4.290 mmol) in dioxane (5 mL) was stirred for 2 h at 100° C. under nitrogen atmosphere. The resulting mixture was concentrated under reduced pressure. The residue was purified by silica gel column chromatography, eluted with PE/EtOAc (1/1) to afford (4R)-1-[4-chloro-6-(morpholin-4-yl)pyridin-2-yl]-4-hydroxypyrrolidin-2-one (550 mg, 78%) as a brown solid. MS ESI calculated for C₁₃H₁₆ClN₃O₃[M+H]⁺, 298.09, found 298.05. ¹H NMR (400 MHz, Chloroform-d) δ 7.83 (s, 1H), 6.36 (s, 1H), 4.60 (s, 1H), 4.19 (dd, J=12.4, 5.2 Hz, 1H), 4.14-4.09 (m, 1H), 3.87-3.79 (m, 4H), 3.52-3.45 (m, 4H), 2.97 (dd, J=17.6, 6.3 Hz, 1H), 2.68-2.62 (m, 1H), 2.10 (s, 1H).

### Example 125: (3S)-N-(3-[2-[(4R)-4-hydroxy-2-oxopyrrolidin-1-yl]-6-(morpholin-4-yl)pyridin-4-yl]-4-methylphenyl)-3-(2,2,2-trifluoroethyl)pyrrolidine-1-carboxamide

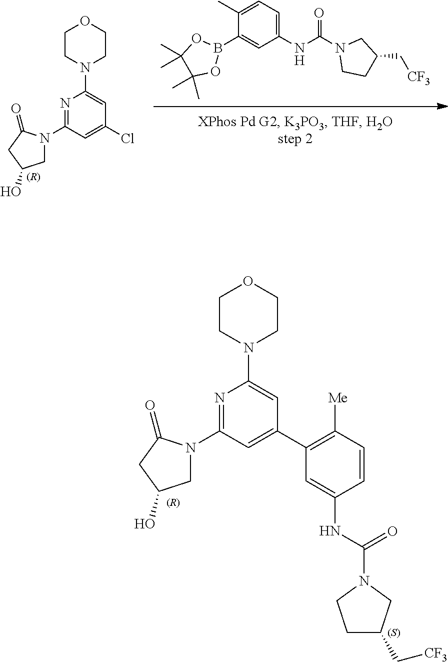

A mixture of (4R)-1-[4-chloro-6-(morpholin-4-yl)pyridin-2-yl]-4-hydroxypyrrolidin-2-one (150 mg, 0.504 mmol), (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (166 mg, 0.403 mmol), 2^{nd} XPhos Precatalyst (40 mg, 0.050 mmol) and K₃PO₄ (214 mg, 1.008 mmol) in THF (1.5 mL) and H₂O (0.2 mL) was stirred for 2 h at 80° C. under nitrogen atmosphere. The reaction was quenched by the addition of water (10 mL). The resulting mixture was extracted with EtOAc (2×20 mL). The combined organic layers were washed with brine (10 mL), dried over anhydrous Na₂SO₄. After filtration, the filtrate was concentrated under reduced pressure. The residue was purified by Prep-TLC with PE/EtOAc/EtOH (8/3/1) to afford (3S)-N-(3-[2-[(4R)-4-hydroxy-2-oxopyrrolidin-1-yl]-6-(morpholin-4-yl)pyridin-4-yl]-4-methylphenyl)-3-(2,2,2-trifluoroethyl)pyrrolidine-1-carboxamide (126 mg, 45%) as an off-white solid. MS ESI calculated for C₂₇H₃₂F₃N₅O₄[M+H]⁺, 548.24, found 548.25. ¹H NMR (400 MHz, DMSO-d₆) δ 8.18 (s, 1H), 7.60 (s, 1H), 7.48 (dd, J=8.4, 2.4 Hz, 1H), 7.38 (d, J=2.4 Hz, 1H), 7.14 (d, J=8.4 Hz, 1H), 6.44 (s, 1H), 5.27 (s, 1H), 4.37-4.35 (m, 1H), 4.09-4.05 (m, 1H), 4.00-3.97 (m, 1H), 3.73-3.70 (m, 5H), 3.68-3.46 (m, 5H), 3.33-3.28 (m, 1H), 3.05-2.91 (m, 1H), 2.89-2.87 (m, 1H), 2.53-2.32 (m, 4H), 2.16 (s, 3H), 2.10-2.07 (m, 1H), 1.68-1.63 (m, 1H). ¹⁹F NMR (376 MHz, DMSO-d₆) δ-63.37 (3F).

### Example 126: (3S)-N-[3-[2-(1,1-dioxo-1lambda6,2-thiazolidin-2-yl)-6-(morpholin-4-yl)pyridin-4-yl]-4-methylphenyl]-3-(2,2,2-trifluoroethyl)pyrrolidine-1-carboxamide

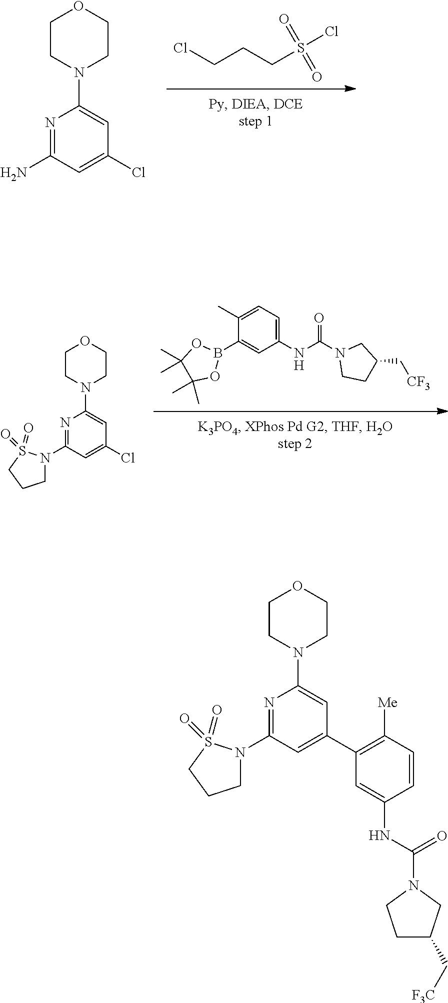 [SMILES: Nc1cc(Cl)cc(N2CCOCC2)n1 ; O=S(=O)(Cl)CCCCl ; O=S1(=O)CCCN1c1cc(Cl)cc(N2CCOCC2)n1 ; Cc1ccc(NC(=O)N2CC[C@@H](CC(F)(F)F)C2)cc1B1OC(C)(C)C(C)(C)O1 ; Cc1ccc(NC(=O)N2CC[C@@H](CC(F)(F)F)C2)cc1-c1cc(N2CCOCC2)nc(N2CCCS2(=O)=O)c1]

Preparation 126A: 2-[6-(morpholin-4-yl)pyridin-2-yl]-1lambda6,2-thiazolidine-1,1-dione

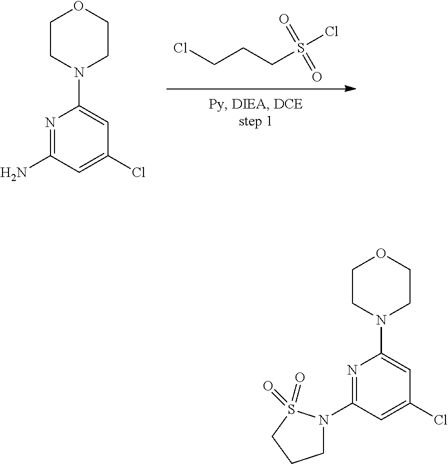 [SMILES: Nc1cc(Cl)cc(N2CCOCC2)n1 ; O=S(=O)(Cl)CCCCl ; O=S1(=O)CCCN1c1cc(Cl)cc(N2CCOCC2)n1]

To a stirred mixture of 4-chloro-6-(morpholin-4-yl)pyridin-2-amine (250 mg, 1.200 mmol), pyridine (0.5 mL) and DIEA (756 mg, 6.000 mmol) in DCE (5 mL) was added 3-chloropropane-1-sulfonyl chloride (414 mg, 2.300 mmol) dropwise at 0° C. The reaction mixture was stirred for 3 h and quenched with sat. Na₂CO₃ (50 mL). The resulting mixture was extracted with EtOAc (3×50 mL). The combined organic layers were washed with brine (50 mL), dried over anhydrous Na₂SO₄. After filtration, the filtrate was concentrated under reduced pressure. The residue was purified by silica gel column chromatography, eluted with PE/EtOAc (1/1) to afford 2-[6-(morpholin-4-yl)pyridin-2-yl]-1lambda6,2-thiazolidine-1,1-dione (200 mg, 34%) as an off-white solid. MS ESI calculated for C₁₂H₁₆ClN₃O₃S [M+H]⁺, 318.06, found 318.05. ¹H NMR (300 MHz, Chloroform-d) δ 6.52 (d, J=1.2 Hz, 1H), 6.29 (d, J=1.2 Hz, 1H), 3.94 (t, J=6.8 Hz, 2H), 3.86-3.74 (m, 4H), 3.51-3.49 (m, 4H), 3.41 (t, J=7.2 Hz, 2H), 2.51-2.46 (m, 2H).

### Example 126: (3S)-N-[3-[2-(1,1-dioxo-1lambda6,2-thiazolidin-2-yl)-6-(morpholin-4-yl)pyridin-4-yl]-4-methylphenyl]-3-(2,2,2-trifluoroethyl)pyrrolidine-1-carboxamide

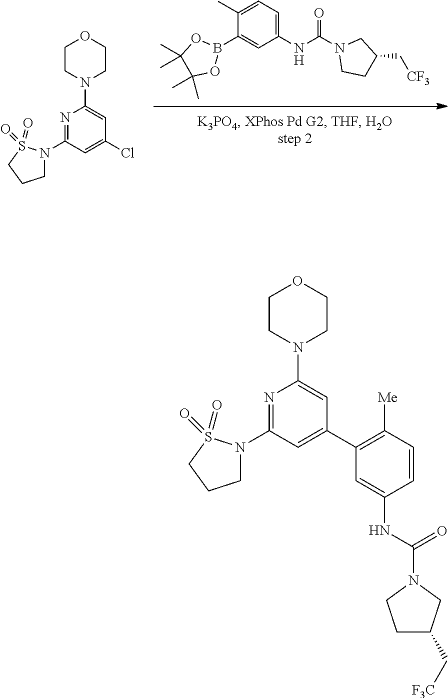 [SMILES: O=S1(=O)CCCN1c1cc(Cl)cc(N2CCOCC2)n1 ; Cc1ccc(NC(=O)N2CC[C@@H](CC(F)(F)F)C2)cc1B1OC(C)(C)C(C)(C)O1 ; Cc1ccc(NC(=O)N2CC[C@@H](CC(F)(F)F)C2)cc1-c1cc(N2CCOCC2)nc(N2CCCS2(=O)=O)c1]

A mixture of 2-[4-chloro-6-(morpholin-4-yl)pyridin-2-yl]-1lambda6,2-thiazolidine-1,1-dione (200 mg, 0.630 mmol), (3S)-N-[4-methyl-3-(4,4,5-trimethyl-1,3,2-dioxaborolan-2-yl)phenyl]-3-(2,2,2-trifluoroethyl)pyrrolidine-1-carboxamide (200 mg, 0.500 mmol), K₃PO₄ (267 mg, 0.130 mmol) and 2^{nd} XPhos Precatalyst (53 mg, 0.006 mmol) in THF (2 mL) and H₂O (0.2 mL) was stirred for 2 h at 80° C. under nitrogen atmosphere. The resulting mixture was concentrated under reduced pressure. The residue was purified by silica gel column chromatography, eluted with PE/EtOAc (1/1) to afford (3S)-N-[3-[2-(1,1-dioxo-1lambda6,2-thiazolidin-2-yl)-6-(morpholin-4-yl)pyridin-4-yl]-4-methylphenyl]-3-(2,2,2-trifluoroethyl)pyrrolidine-1-carboxamide (115 mg, 32%) as an off-white solid. MS ESI calculated for C₂₆H₃₂F₃N₅O₄S [M+H]⁺, 568.21, found 568.15. ¹H NMR (400 MHz, Chloroform-d) δ 7.43 (dd, J=8.0, 2.4 Hz, 1H), 7.24-7.12 (m, 2H), 6.48 (s, 1H), 6.25 (s, 1H), 6.18 (s, 1H), 4.00 (t, J=6.8 Hz, 2H), 3.93-3.69 (m, 5H), 3.67-3.62 (m, 1H), 3.58-3.48 (m, 4H), 3.48-3.37 (m, 3H), 3.15-3.10 (m, 1H), 2.62-2.47 (m, 3H), 2.34-2.21 (m, 6H), 1.81-1.70 (m, 1H). ¹⁹F NMR (376 MHz, Chloroform-d) δ-64.95 (3F).

### Example 127: (S)-N-(3-(2-((R)-4-amino-2-oxopyrrolidin-1-yl)-6-morpholinopyridin-4-yl)-4-methylphenyl)-3-(2,2,2-trifluoroethyl)pyrrolidine-1-carboxamide

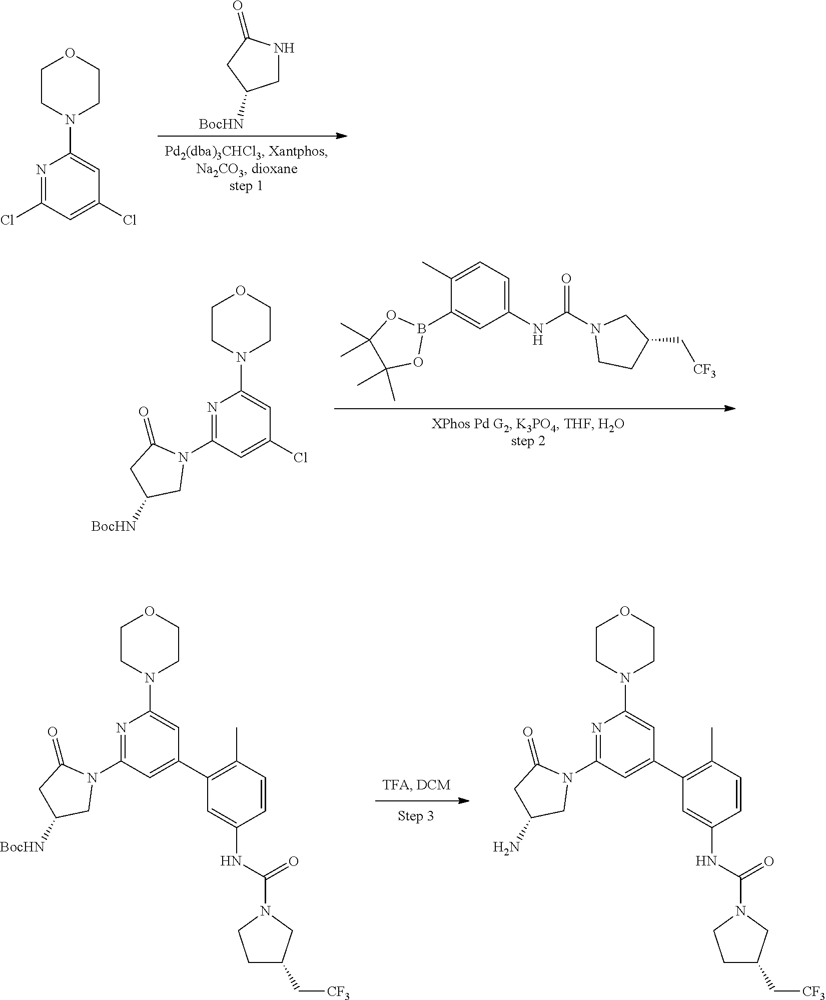 [SMILES: Clc1cc(Cl)nc(N2CCOCC2)c1 ; CC(C)(C)OC(=O)N[C@H]1CNC(=O)C1 ; CC(C)(C)OC(=O)N[C@@H]1CC(=O)N(c2cc(Cl)cc(N3CCOCC3)n2)C1 ; Cc1ccc(NC(=O)N2CC[C@@H](CC(F)(F)F)C2)cc1B1OC(C)(C)C(C)(C)O1 ; Cc1ccc(NC(=O)N2CC[C@@H](CC(F)(F)F)C2)cc1-c1cc(N2CCOCC2)nc(N2C[C@H](NC(=O)OC(C)(C)C)CC2=O)c1 ; Cc1ccc(NC(=O)N2CC[C@@H](CC(F)(F)F)C2)cc1-c1cc(N2CCOCC2)nc(N2C[C@H](N)CC2=O)c1]

Preparation 127A: tert-butyl (R)-(1-(4-chloro-6-morpholinopyridin-2-yl)-5-oxopyrrolidin-3-yl)carbamate

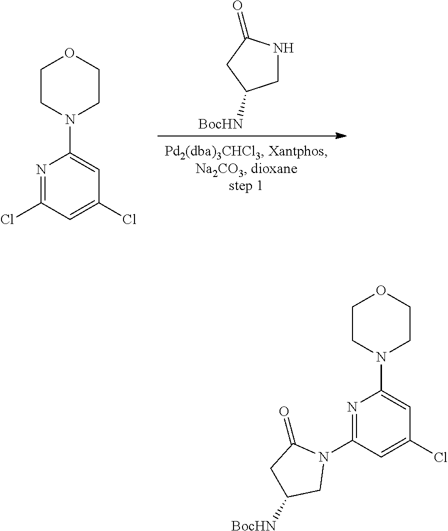 [SMILES: Clc1cc(Cl)nc(N2CCOCC2)c1 ; CC(C)(C)OC(=O)N[C@H]1CNC(=O)C1 ; CC(C)(C)OC(=O)N[C@@H]1CC(=O)N(c2cc(Cl)cc(N3CCOCC3)n2)C1]

A mixture of 4-(4,6-dichloropyridin-2-yl)morpholine (500 mg, 2.160 mmol) and tert-butyl (R)-(5-oxopyrrolidin-3-yl)carbamate (648 mg, 3.240 mmol), dioxane (5 mL), Pd₂(dba)₃·CHCl₃ (228 mg, 0.220 mmol), Xantphos (254 mg, 0.440 mmol) and Na₂CO₃ (687 mg, 6.480 mmol) was stirred for 2 h at 100° C. under nitrogen atmosphere. The resulting mixture was concentrated under reduced pressure. The residue was purified by silica gel column chromatography, eluted with EtOAc/PE (1/4) to afford tert-butyl (R)-(1-(4-chloro-6-morpholinopyridin-2-yl)-5-oxopyrrolidin-3-yl)carbamate (567.8 mg, 67%) as a yellow solid. MS ESI calculated for C₁₈H₂₅ClN₄O₄[M+H]⁺, 397.16, found 397.10. ¹H NMR (400 MHz, Chloroform-d₆) δ 7.76 (s, 1H), 6.35 (s, 1H), 4.92 (s, 1H), 4.37 (s, 1H), 4.26 (dd, J=12.0, 6.4 Hz, 1H), 3.94 (dd, J=12.0, 3.2 Hz, 1H), 3.82-3.76 (m, 4H), 3.48-3.41 (m, 4H), 3.03-2.97 (m, 1H), 2.56-2.51 (m, 1H), 1.45 (s, 9H).

Preparation 127B: tert-butyl ((R)-1-(4-(2-methyl-5-((S)-3-(2,2,2-trifluoroethyl)pyrrolidine-1-carboxamido)phenyl)-6-morpholinopyridin-2-yl)-5-oxopyrrolidin-3-yl)carbamate

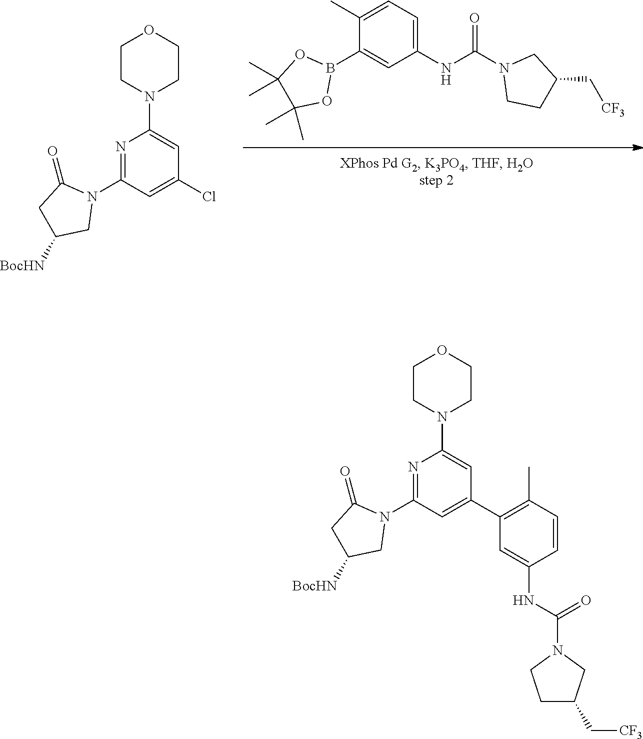 [SMILES: CC(C)(C)OC(=O)N[C@@H]1CC(=O)N(c2cc(Cl)cc(N3CCOCC3)n2)C1 ; Cc1ccc(NC(=O)N2CC[C@@H](CC(F)(F)F)C2)cc1B1OC(C)(C)C(C)(C)O1 ; Cc1ccc(NC(=O)N2CC[C@@H](CC(F)(F)F)C2)cc1-c1cc(N2CCOCC2)nc(N2C[C@H](NC(=O)OC(C)(C)C)CC2=O)c1]

A mixture of tert-butyl (R)-(1-(4-chloro-6-morpholinopyridin-2-yl)-5-oxopyrrolidin-3-yl)carbamate (200 mg, 0.505 mmol) and (S)-N-(4-methyl-3-(4,4,5,5-tetramethyl-1,3,2-dioxaborolan-2-yl)phenyl)-3-(2,2,2-trifluoroethyl)pyrrolidine-1-carboxamide (250 mg, 0.606 mmol), 2^{nd} XPhos Precatalyst (40 mg, 0.051 mmol) and K₃PO₄ (212 mg, 1.010 mmol) in THF (5 mL) and H₂O (0.5 mL) was stirred for 3 h at 80° C. under nitrogen atmosphere. The resulting mixture was concentrated under reduced pressure. The residue was purified by silica gel column chromatography, eluted with EtOAc/PE (1/1) to afford tert-butyl ((R)-1-(4-(2-methyl-5-((S)-3-(2,2,2-trifluoroethyl)pyrrolidine-1-carboxamido)phenyl)-6-morpholinopyridin-2-yl)-5-oxopyrrolidin-3-yl)carbamate (293 mg, 90%) as a light yellow solid. MS ESI calculated for C₃₂H₄₁F₃N₆O₅⁺[M+H]⁺, 647.31, found 647.20.

### Example 127: (S)-N-(3-(2-((R)-4-amino-2-oxopyrrolidin-1-yl)-6-morpholinopyridin-4-yl)-4-methylphenyl)-3-(2,2,2-trifluoroethyl)pyrrolidine-1-carboxamide

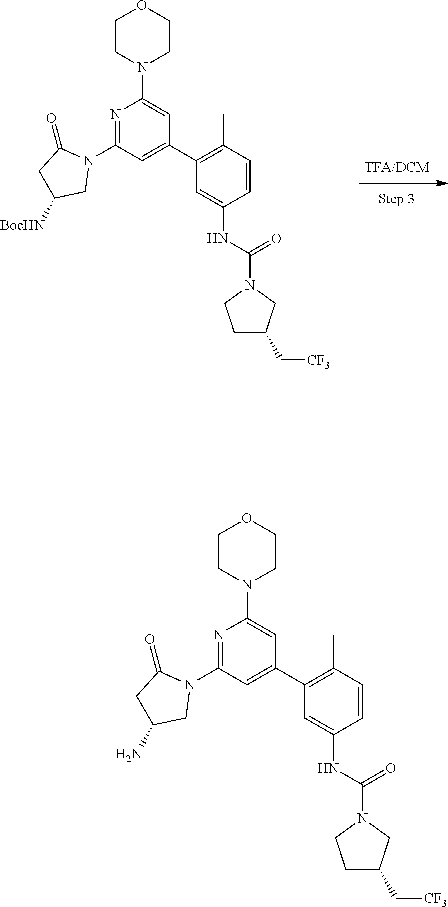 [SMILES: Cc1ccc(NC(=O)N2CC[C@@H](CC(F)(F)F)C2)cc1-c1cc(N2CCOCC2)nc(N2C[C@H](NC(=O)OC(C)(C)C)CC2=O)c1 ; Cc1ccc(NC(=O)N2CC[C@@H](CC(F)(F)F)C2)cc1-c1cc(N2CCOCC2)nc(N2C[C@H](N)CC2=O)c1]

A mixture of tert-butyl ((R)-1-(4-(2-methyl-5-((S)-3-(2,2,2-trifluoroethyl)pyrrolidine-1-carboxamido)phenyl)-6-morpholinopyridin-2-yl)-5-oxopyrrolidin-3-yl)carbamate (200 mg, 0.309 mmol), DCM (5.0 mL) and TFA (0.5 mL) was stirred for 30 min at room temperature. The resulting mixture was concentrated under reduced pressure. The residue was purified by Prep-HPLC with following conditions: Column: Spherical C18, 20-40 μm, 120 g; Mobile Phase A: water (plus 10 mM NH₄CO₃); Mobile Phase B: CH₃CN; Flow rate: 60 mL/min; Gradient: 35% B to 65% B; Detector: 220 nm to afford (S)-N-(3-(2-((R)-4-amino-2-oxopyrrolidin-1-yl)-6-morpholinopyridin-4-yl)-4-methylphenyl)-3-(2,2,2-trifluoroethyl)pyrrolidine-1-carboxamide (90.5 mg, 53%) as a white solid. MS ESI calculated for C₂₇H₃₃F₃N₆O₃[M+H]⁺, 547.26, found 547.30. ¹H NMR (400 MHz, Chloroform-d₆) δ 7.75 (s, 1H), 7.36 (dd, J=8.4, 2.4 Hz, 1H), 7.22 (d, J=2.4 Hz, 1H), 7.16 (d, J=8.4 Hz, 1H), 6.31 (s, 1H), 6.20 (s, 1H), 4.32-4.27 (m, 1H), 3.93-3.87 (m, 1H), 3.82-3.78 (m, 6H), 3.63-3.60 (m, 1H), 3.49-3.37 (m, 5H), 3.12-3.08 (m, 1H), 2.94-2.90 (m, 1H), 2.60-2.43 (m, 2H), 2.29-2.20 (m, 6H), 1.76-1.63 (m, 3H). ¹⁹F NMR (376 MHz, Chloroform-d₆) δ-64.94 (3F).

### Examples 128 and 129: (3S)-N-(3-{2-[(4R)-3,3-difluoro-4-hydroxypyrrolidin-1-yl]-6-(morpholin-4-yl)pyridin-4-yl}-4-methylphenyl)-3-(2,2,2-trifluoroethyl)pyrrolidine-1-carboxamide and (3S)-N-(3-{2-[(4S)-3,3-difluoro-4-hydroxypyrrolidin-1-yl]-6-(morpholin-4-yl)pyridin-4-yl}-4-methylphenyl)-3-(2,2,2-trifluoroethyl)pyrrolidine-1-carboxamide

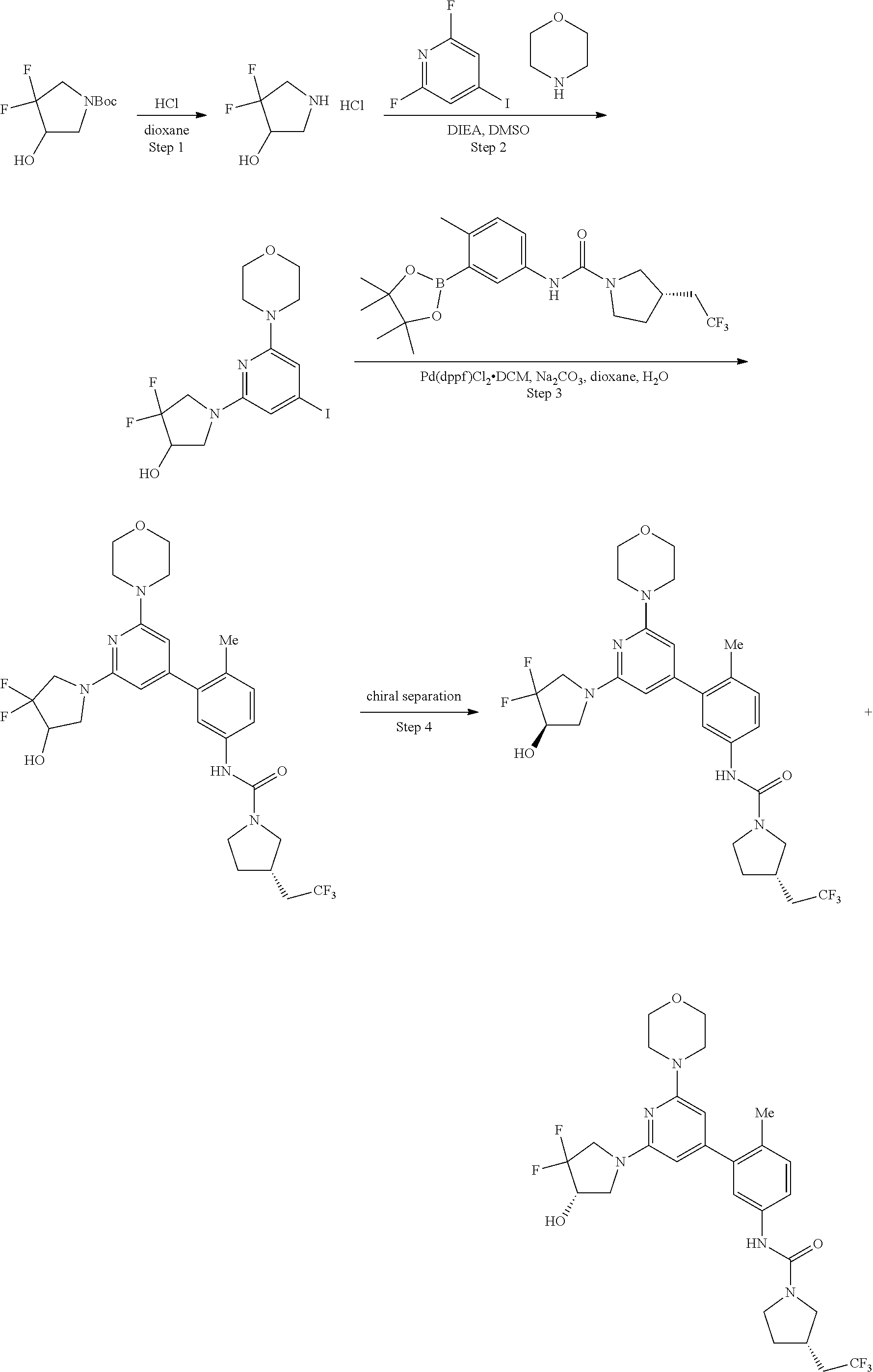

Preparation 128A: 4,4-difluoropyrrolidin-3-ol hydrochloride

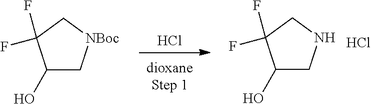 [SMILES: CC(C)(C)OC(=O)N1CC(O)C(F)(F)C1 ; Cl ; OC1CNCC1(F)F ; Cl]

To a stirred solution of tert-butyl 3,3-difluoro-4-hydroxypyrrolidine-1-carboxylate (300 mg, 1.344 mmol) in DCM (5 mL) was added HCl in dioxane (1 mL, 1 M) dropwise at 0° C. The resulting mixture was stirred for 2 h at room temperature. The resulting mixture was concentrated under reduced pressure to afford 4,4-difluoropyrrolidin-3-ol hydrochloride (350 mg, crude) as yellow oil. C₄H₈ClF₂NO, ¹H NMR (400 MHz, DMSO-d₆) δ 4.33-4.31 (m, 1H), 3.59-3.52 (m, 2H), 3.51-3.45 (m, 1H), 3.29-3.26 (m, 1H).

Preparation 128B: 4-iodo-N-(4-methyloxan-4-yl)-6-(morpholin-4-yl)pyridin-2-amine

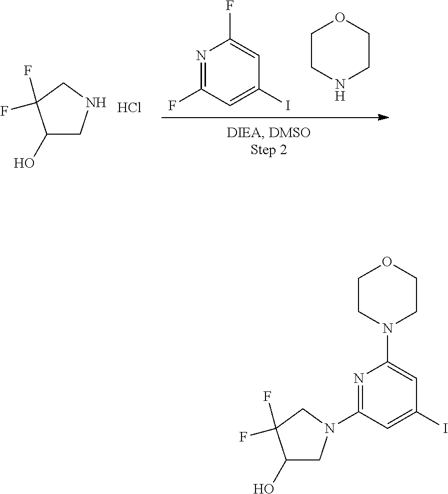

To a stirred solution of 2,6-difluoro-4-iodopyridine (350 mg, 1.452 mmol) and 4,4-difluoropyrrolidin-3-ol hydrochloride (214 mg, 1.742 mmol) in DMSO (5 mL) was added DIEA (412 mg, 3.194 mmol) at room temperature. The resulting mixture was stirred for 4 h at 80° C. To this was added morpholine (632 mg, 7.260 mmol). The resulting mixture was stirred for 16 h at 80° C. The mixture was allowed to cool down to room temperature. The resulting mixture was diluted with H₂O (50 mL) and extracted with EtOAc (3×50 mL). The combined organic layers were washed with brine (50 mL), dried over anhydrous Na₂SO₄. After filtration, the filtrate was concentrated under reduced pressure. The residue was purified by silica gel column chromatography, eluted with PE/EtOAc (4/1) to afford 4-iodo-N-(4-methyloxan-4-yl)-6-(morpholin-4-yl)pyridin-2-amine (240 mg, 40%) as a yellow solid. MS ESI calculated for C₁₃H₁₆F₂IN₃O₂[M+H]⁺, 412.03, found 412.05. ¹H NMR (400 MHz, Chloroform-d) δ 6.36 (s, 1H), 6.14 (s, 1H), 4.40-4.36 (m, 1H), 3.88-3.74 (m, 7H), 3.55-3.53 (m, 1H), 3.50-3.42 (m, 4H).

Preparation 128C: (3S)-N-[3-[2-(3,3-difluoro-4-hydroxypyrrolidin-1-yl)-6-(morpholin-4-yl)pyridin-4-yl]-4-methylphenyl]-3-(2,2,2-trifluoroethyl)pyrrolidine-1-carboxamide

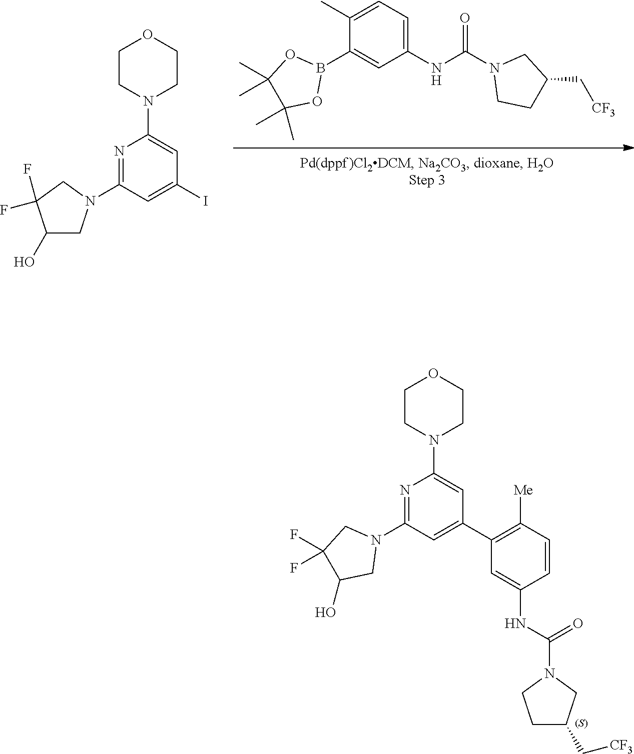

A mixture of 4,4-difluoro-1-[4-iodo-6-(morpholin-4-yl)pyridin-2-yl]pyrrolidin-3-ol (240 mg, 0.584 mmol) and (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (240 mg, 0.582 mmol), 1,4-dioxane (2.4 mL), H₂O (0.6 mL), Pd(dppf)Cl₂·CH₂Cl₂ (47 mg, 0.058 mmol) and Na₂CO₃ (185 mg, 1.752 mmol) was stirred for 2 h at 60° C. under nitrogen atmosphere. The reaction was quenched with water (50 mL). The resulting mixture was extracted with EtOAc (3×50 mL). The combined organic layers were washed with brine (30 mL), dried over anhydrous Na₂SO₄. After filtration, the filtrate was concentrated under reduced pressure. The residue was purified by silica gel column chromatography, eluted with PE/EtOAc/EtOH (2/3/1) to afford (3S)-N-[3-[2-(3,3-difluoro-4-hydroxypyrrolidin-1-yl)-6-(morpholin-4-yl)pyridin-4-yl]-4-methylphenyl]-3-(2,2,2-trifluoroethyl)pyrrolidine-1-carboxamide (265 mg, 79%) as a white solid. MS ESI calculated for C₂₇H₃₂F₅N₅O₃[M+H]⁺, 570.24, found 570.30.

### Examples 128 and 129: (3S)-N-(3-{2-[(4R)-3,3-difluoro-4-hydroxypyrrolidin-1-yl]-6-(morpholin-4-yl)pyridin-4-yl}-4-methylphenyl)-3-(2,2,2-trifluoroethyl)pyrrolidine-1-carboxamide and (3S)-N-(3-{2-[(4S)-3,3-difluoro-4-hydroxypyrrolidin-1-yl]-6-(morpholin-4-yl)pyridin-4-yl}-4-methylphenyl)-3-(2,2,2-trifluoroethyl)pyrrolidine-1-carboxamide

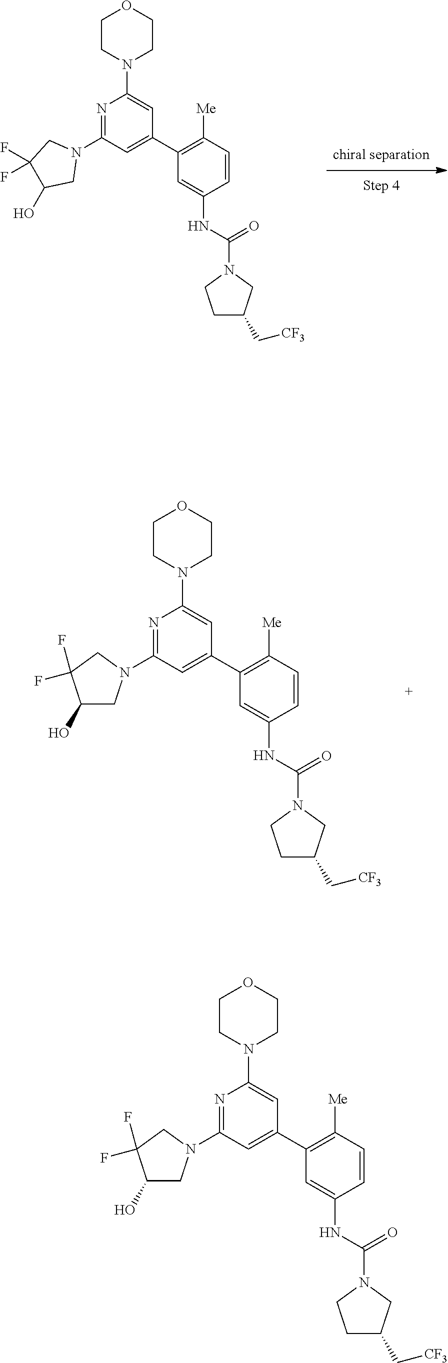 [SMILES: Cc1ccc(NC(=O)N2CC[C@@H](CC(F)(F)F)C2)cc1-c1cc(N2CCOCC2)nc(N2CC(O)C(F)(F)C2)c1 ; Cc1ccc(NC(=O)N2CC[C@@H](CC(F)(F)F)C2)cc1-c1cc(N2CCOCC2)nc(N2C[C@@H](O)C(F)(F)C2)c1 ; Cc1ccc(NC(=O)N2CC[C@@H](CC(F)(F)F)C2)cc1-c1cc(N2CCOCC2)nc(N2C[C@H](O)C(F)(F)C2)c1]

(3S)-N-{3-[2-(3,3-difluoro-4-hydroxypyrrolidin-1-yl)-6-(morpholin-4-yl)pyridin-4-yl]-4-methylphenyl}-3-(2,2,2-trifluoroethyl)pyrrolidine-1-carboxamide (265 mg, 0.465 mmol) was resolved by Chiral Prep HPLC with the following conditions: Column: (R,R)-WHELK-O1-Kromasil, 2.11*25 cm, 5 m; Mobile Phase A: Hexand (0.5% 2M NH₃-MeOH), Mobile Phase B: EtOH; A:B=50:50; 220/254 nm.

RT₁: 29.71 min yielded 55 mg (20%) of a white solid. MS ESI calculated for C₂₇H₃₂F₅N₅O₃ [M+H]⁺,570.24, found 570.20. ¹H NMR (400 MHz, DMSO-d₆) δ 8.12 (s, 1H), 7.43 (dd, J=8.4, 2.4 Hz, 1H), 7.33 (d, J=2.0 Hz, 1H), 7.11 (d, J=8.4 Hz, 1H), 6.05 (d, J=5.2 Hz, 1H), 5.95 (s, 1H), 5.73 (s, 1H), 4.35-4.31 (m, 1H), 3.95-3.61 (m, 9H), 3.52-3.49 (m, 1H), 3.43-3.3.30 (m, 5H), 3.02 (t, J=9.6 Hz, 1H), 2.42-2.30 (m, 3H), 2.17 (s, 3H), 2.10-2.06 (m, 1H), 1.66-1.55 (m, 1H). ¹⁹F NMR (376 MHz, DMSO-d₆) δ-66.36 (3F), −109.20 (d, J=230.5 Hz, 1F), −120.58 (d, J=230.5 Hz, 1F).

RT₂: 39.58 min yielded 55 mg (20%) as a white solid. MS ESI calculated for C₂₇H₃₂F₅N₅O₃ [M+H]⁺, 570.24, found 570.20. ¹H NMR (400 MHz, DMSO-d₆) δ 8.12 (s, 1H), 7.43 (dd, J=8.4, 2.4 Hz, 1H), 7.33 (d, J=2.0 Hz, 1H), 7.11 (d, J=8.4 Hz, 1H), 5.95 (s, 1H), 5.73 (s, 1H), 4.35-4.31 (m, 1H), 3.95-3.61 (m, 9H), 3.53-3.51 (m, 1H), 3.41-3.29 (m, 5H), 3.02 (t, J=9.6 Hz, 1H), 2.42-2.37 (m, 3H), 2.17 (s, 3H), 2.12-2.07 (m, 1H), 1.66-1.62 (m, 1H). ¹⁹F NMR (376 MHz, DMSO-d₆) δ-58.61 (3F), −104.46 (d, J=230.5 Hz, 1F), −115.83 (d, J=230.5 Hz, 1F).

### Examples 130 and 131: (3S)-N-(3-{2-[(1R)-1-amino-3-azabicyclo[3.1.0]hexan-3-yl]-6-(morpholin-4-yl)pyridin-4-yl}-4-methylphenyl)-3-(2,2,2-trifluoroethyl)pyrrolidine-1-carboxamide and (3S)-N-(3-{2-[(1S)-1-amino-3-azabicyclo[3.1.0]hexan-3-yl]-6-(morpholin-4-yl)pyridin-4-yl}-4-methylphenyl)-3-(2,2,2-trifluoroethyl)pyrrolidine-1-carboxamide

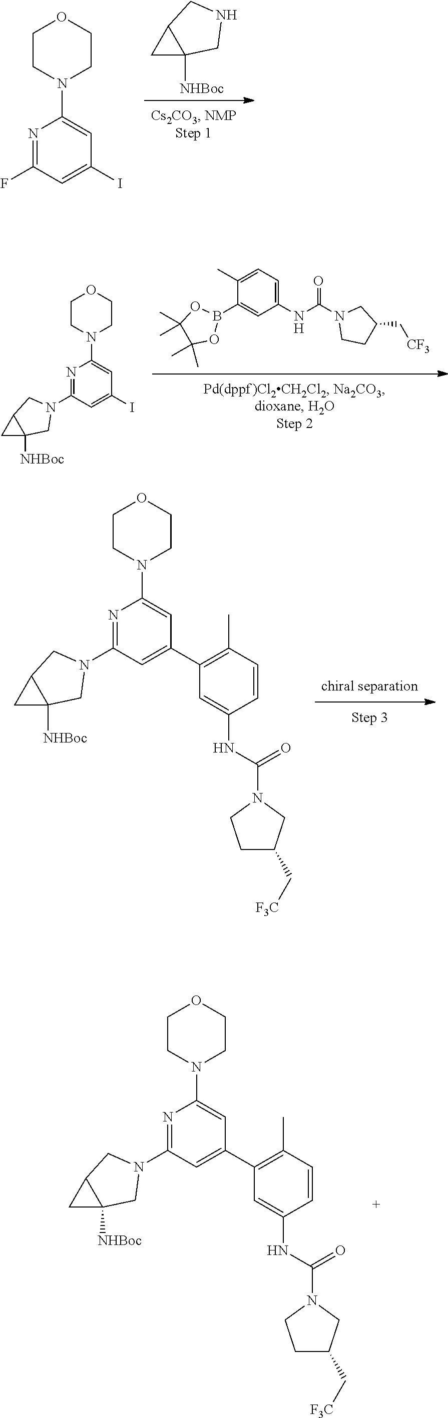 [SMILES: Fc1cc(I)cc(N2CCOCC2)n1 ; CC(C)(C)OC(=O)NC12CNCC1C2 ; CC(C)(C)OC(=O)NC12CC1CN(c1cc(I)cc(N3CCOCC3)n1)C2 ; Cc1ccc(NC(=O)N2CC[C@@H](CC(F)(F)F)C2)cc1B1OC(C)(C)C(C)(C)O1 ; Cc1ccc(NC(=O)N2CC[C@@H](CC(F)(F)F)C2)cc1-c1cc(N2CCOCC2)nc(N2CC3CC3(NC(=O)OC(C)(C)C)C2)c1 ; Cc1ccc(NC(=O)N2CC[C@@H](CC(F)(F)F)C2)cc1-c1cc(N2CCOCC2)nc(N2CC3C[C@]3(NC(=O)OC(C)(C)C)C2)c1]

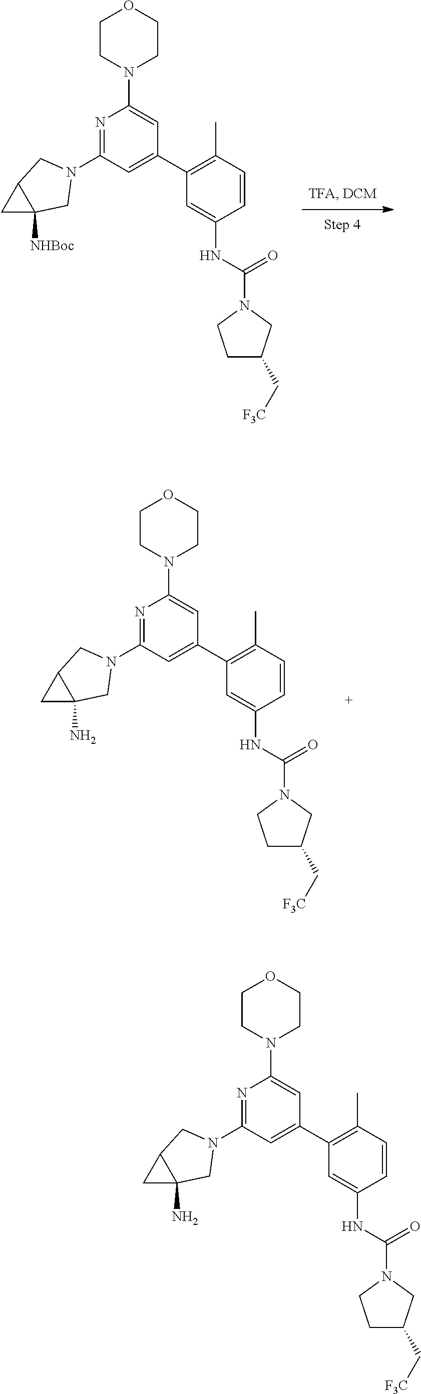 [SMILES: Cc1ccc(NC(=O)N2CC[C@@H](CC(F)(F)F)C2)cc1-c1cc(N2CCOCC2)nc(N2CC3C[C@@]3(NC(=O)OC(C)(C)C)C2)c1 ; Cc1ccc(NC(=O)N2CC[C@@H](CC(F)(F)F)C2)cc1-c1cc(N2CCOCC2)nc(N2CC3C[C@]3(N)C2)c1 ; Cc1ccc(NC(=O)N2CC[C@@H](CC(F)(F)F)C2)cc1-c1cc(N2CCOCC2)nc(N2CC3C[C@@]3(N)C2)c1]

Preparation 130A: tert-butyl N-[3-[4-iodo-6-(morpholin-4-yl)pyridin-2-yl]-3-azabicyclo[3.1.0]hexan-1-yl]carbamate

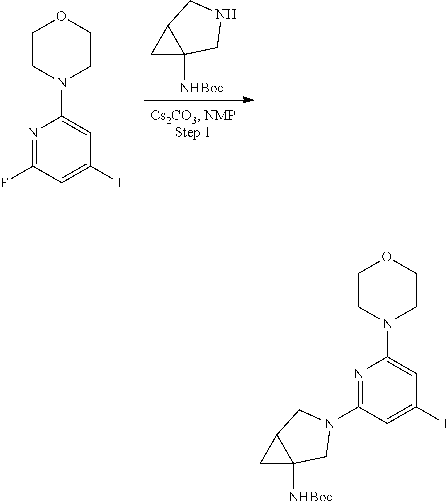 [SMILES: Fc1cc(I)cc(N2CCOCC2)n1 ; CC(C)(C)OC(=O)NC12CNCC1C2 ; CC(C)(C)OC(=O)NC12CC1CN(c1cc(I)cc(N3CCOCC3)n1)C2]

To a stirred mixture of 4-(6-fluoro-4-iodopyridin-2-yl)morpholine (400 mg, 1.298 mmol) and tert-butyl N-[3-azabicyclo[3.1.0]hexan-1-yl]carbamate (309 mg, 1.558 mmol) in NMP (4 mL) was added Cs₂CO₃ (1.27 g, 3.895 mmol). The reaction mixture was stirred for 2 h at 130° C. under nitrogen atmosphere. The reaction was quenched with water (20 mL). The resulting mixture was extracted with EtOAc (3×20 mL). The combined organic layers were washed with brine (20 mL), dried over anhydrous Na₂SO₄. After filtration, the filtrate was concentrated under reduced pressure. The residue was purified by silica gel column chromatography, eluted with PE/EtOAc (5/1) to afford tert-butyl N-[3-[4-iodo-6-(morpholin-4-yl)pyridin-2-yl]-3-azabicyclo[3.1.0]hexan-1-yl]carbamate (100 mg, 16%) as a light yellow solid. MS ESI calculated for C₁₉H₂₇IN₄O₃[M+H]⁺ 487.11; found 487.05. ¹H NMR (400 MHz, Chloroform-d) δ 6.30 (s, 1H), 6.15 (s, 1H), 5.05 (brs, 1H), 3.93-3.91 (m, 1H), 3.85-3.78 (m, 4H), 3.62-3.60 (m, 2H), 3.45-3.41 (m, 5H), 1.81-1.79 (m, 1H), 1.47 (s, 9H), 1.10-1.08 (m, 1H), 0.76-0.74 (m, 1H).

Preparation 130B: tert-butyl N-[3-(4-[2-methyl-5-[(3S)-3-(2,2,2-trifluoroethyl)pyrrolidine-1-carbonylamino]phenyl]-6-(morpholin-4-yl)pyridin-2-yl)-3-azabicyclo[3.1.0]hexan-1-yl]carbamate

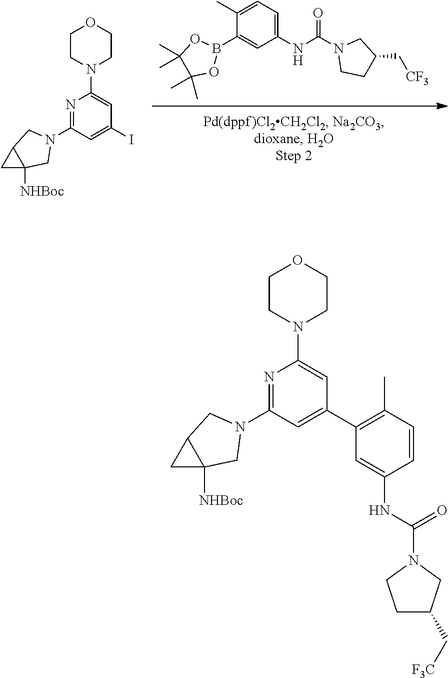 [SMILES: CC(C)(C)OC(=O)NC12CC1CN(c1cc(I)cc(N3CCOCC3)n1)C2 ; Cc1ccc(NC(=O)N2CC[C@@H](CC(F)(F)F)C2)cc1B1OC(C)(C)C(C)(C)O1 ; Cc1ccc(NC(=O)N2CC[C@@H](CC(F)(F)F)C2)cc1-c1cc(N2CCOCC2)nc(N2CC3CC3(NC(=O)OC(C)(C)C)C2)c1]

To a stirred mixture of tert-butyl (3-(4-iodo-6-morpholinopyridin-2-yl)-3-azabicyclo[3.1.0]hexan-1-yl)carbamate (150 mg, 0.316 mmol) and (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (130 mg, 0.316 mmol) in 1,4-dioxane (2 mL) and H₂O (0.5 mL) were added Na₂CO₃ (100 mg, 0.949 mmol) and Pd(dppf)Cl₂·CH₂Cl₂ (26 mg, 0.032 mmol) was stirred for 16 h in portions at 60° C. under nitrogen atmosphere. The reaction was quenched with water (10 mL) at room temperature. The resulting mixture was extracted with EtOAc (3×20 mL). The combined organic layers were washed with brine (10 mL), dried over anhydrous Na₂SO₄. After filtration, the filtrate was concentrated under reduced pressure. The residue was purified by Prep-TLC with PE/EtOAc (1/1) to afford tert-butyl N-[3-(4-[2-methyl-5-[(3S)-3-(2,2,2-trifluoroethyl)pyrrolidine-1-carbonylamino]phenyl]-6-(morpholin-4-yl)pyridin-2-yl)-3-azabicyclo[3.1.0]hexan-1-yl]carbamate (144 mg, 71%) as an off-white solid. MS ESI calculated for C₃₃H₄₃F₃N₆O₄[M+H]⁺ 645.33; found 645.25.

Preparations 130C/131C: tert-butyl N-[(1R)-3-(4-{2-methyl-5-[(3S)-3-(2,2,2-trifluoroethyl)pyrrolidine-1-carbonylamino]phenyl}-6-(morpholin-4-yl)pyridin-2-yl)-3-azabicyclo[3.1.0]hexan-1-yl]carbamate and tert-butyl N-[(1S)-3-(4-{2-methyl-5-[(3S)-3-(2,2,2-trifluoroethyl)pyrrolidine-1-carbonylamino]phenyl}-6-(morpholin-4-yl)pyridin-2-yl)-3-azabicyclo[3.1.0]hexan-1-yl]carbamate

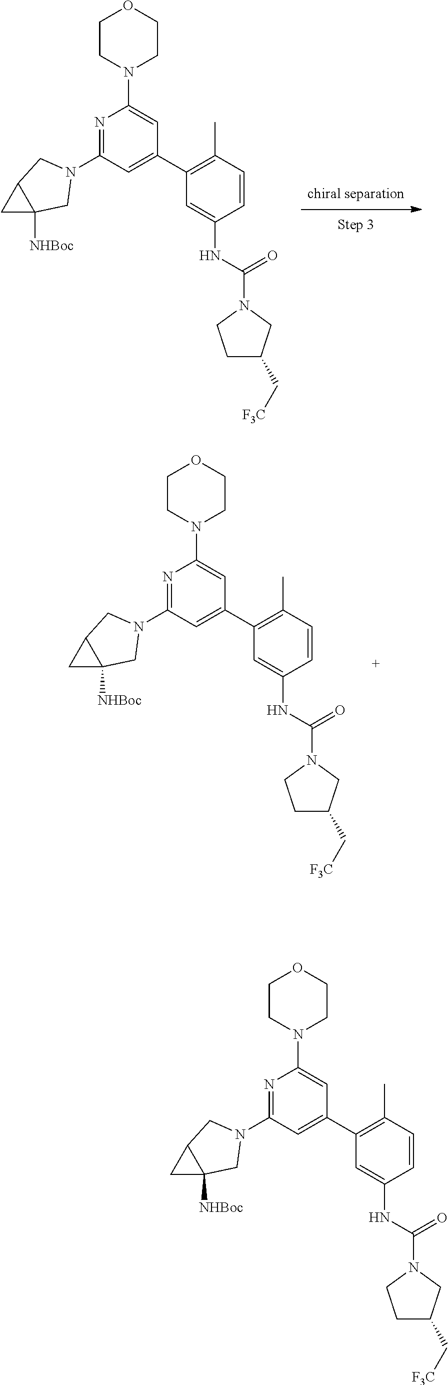 [SMILES: Cc1ccc(NC(=O)N2CC[C@@H](CC(F)(F)F)C2)cc1-c1cc(N2CCOCC2)nc(N2CC3CC3(NC(=O)OC(C)(C)C)C2)c1 ; Cc1ccc(NC(=O)N2CC[C@@H](CC(F)(F)F)C2)cc1-c1cc(N2CCOCC2)nc(N2CC3C[C@]3(NC(=O)OC(C)(C)C)C2)c1 ; Cc1ccc(NC(=O)N2CC[C@@H](CC(F)(F)F)C2)cc1-c1cc(N2CCOCC2)nc(N2CC3C[C@@]3(NC(=O)OC(C)(C)C)C2)c1]

tert-butyl N-[3-(4-{2-methyl-5-[(3S)-3-(2,2,2-trifluoroethyl)pyrrolidine-1-carbonylamino]phenyl}-6-(morpholin-4-yl)pyridin-2-yl)-3-azabicyclo[3.1.0]hexan-1-yl]carbamate (144 mg, 0.223 mmol) was resolved by chiral Prep-HPLC with the following conditions: Column: (R,R)-WHELK-O1-Kromasil, 2.11×25 cm, 5 m; Mobile Phase A: MTBE (0.5% 2M NH₃-MeOH), Mobile Phase B: EtOH; Flow rate:20 mL/min; Gradient: 10% B; 220/254 nm. RT₁: 20.89 min yielded 67 mg (46%) of Preparation 130C. RT₂: 24.627 min yielded 76 mg (53%) of Preparation 131C as an off-white solid.

### Examples 130/131: (3S)-N-(3-{2-[(1R)-1-amino-3-azabicyclo[3.1.0]hexan-3-yl]-6-(morpholin-4-yl)pyridin-4-yl}-4-methylphenyl)-3-(2,2,2-trifluoroethyl)pyrrolidine-1-carboxamide and (3S)-N-(3-{2-[(1S)-1-amino-3-azabicyclo[3.1.0]hexan-3-yl]-6-(morpholin-4-yl)pyridin-4-yl}-4-methylphenyl)-3-(2,2,2-trifluoroethyl)pyrrolidine-1-carboxamide

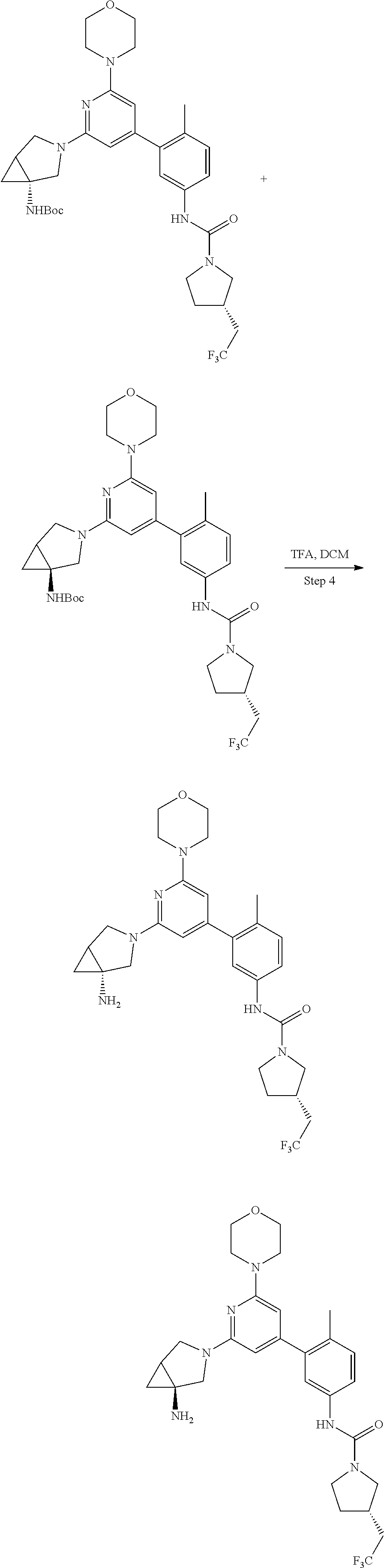 [SMILES: Cc1ccc(NC(=O)N2CC[C@@H](CC(F)(F)F)C2)cc1-c1cc(N2CCOCC2)nc(N2CC3C[C@]3(NC(=O)OC(C)(C)C)C2)c1 ; Cc1ccc(NC(=O)N2CC[C@@H](CC(F)(F)F)C2)cc1-c1cc(N2CCOCC2)nc(N2CC3C[C@@]3(NC(=O)OC(C)(C)C)C2)c1 ; Cc1ccc(NC(=O)N2CC[C@@H](CC(F)(F)F)C2)cc1-c1cc(N2CCOCC2)nc(N2CC3C[C@]3(N)C2)c1 ; Cc1ccc(NC(=O)N2CC[C@@H](CC(F)(F)F)C2)cc1-c1cc(N2CCOCC2)nc(N2CC3C[C@@]3(N)C2)c1]

A mixture of Preparation 130C (60 mg, 0.093 mmol) in DCM (1.5 mL) and TFA (0.3 mL) was stirred for 1 h at room temperature. The resulting mixture was concentrated under reduced pressure. The residue was purified by reverse flash chromatography with the following conditions: column, C₁₈ column; mobile phase, CH₃CN in water, 10% to 50%; detector, UV 254 nm to afford Example 130 (34.2 mg, 67%) as an off-white solid. MS ESI calculated for C₂₈H₃₅F₃N₆O₂[M+H]⁺ 545.28; found 545.20. ¹H NMR (300 MHz, Chloroform-d) δ 7.40-7.38 (m, 1H), 7.28-7.17 (m, 2H), 6.11 (s, 1H), 5.85 (s, 1H), 5.68 (s, 1H), 3.95 (d, J=9.6 Hz, 1H), 3.85-3.82 (m, 5H), 3.79-3.70 (m, 2H), 3.69-3.43 (m, 6H), 3.34 (d, J=9.6 Hz, 1H), 3.12 (t, J=9.6 Hz, 1H), 2.65-2.50 (m, 1H), 2.30-2.23 (m, 6H), 1.57-1.53 (m, 2H), 1.06-1.01 (m, 1H), 0.69-0.66 (m, 1H). ¹⁹F NMR (282 MHz, Chloroform-d) δ-64.95 (3F).

A mixture of Preparation 131C in DCM (1.5 mL) and TFA (0.3 mL) was stirred for 1 h at room temperature under air atmosphere. The resulting mixture was concentrated under reduced pressure. The residue was purified by reverse flash chromatography with the following conditions: column, silica gel; mobile phase, MeCN in water, 10% to 50% gradient in 10 min; detector, UV 254 nm to afford Example 131 (40.3 mg, 68%) as an off-white solid. MS ESI calculated for C₂₈H₃₅F₃N₆O₂[M+H]⁺ 545.28; found 545.25. ¹H NMR (400 MHz, Chloroform-d) δ 7.40-7.38 (m, 1H), 7.28-7.17 (m, 2H), 6.11 (s, 1H), 5.83 (s, 1H), 5.66 (s, 1H), 3.93 (d, J=9.6 Hz, 1H), 3.84-3.78 (m, 5H), 3.68-3.52 (m, 2H), 3.50-3.40 (m, 6H), 3.33 (d, J=10.0 Hz, 1H), 3.12 (t, J=9.6 Hz, 1H), 2.58-2.50 (m, 1H), 2.28-2.18 (m, 6H), 1.57-1.53 (m, 1H), 1.06-1.01 (m, 1H), 0.69-0.66 (m, 1H). ¹⁹F NMR (376 MHz, Chloroform-d) δ-64.95 (3F).

### Example 132: (S)-N-(3-(2-((R)-4-amino-2-oxopyrrolidin-1-yl)-6-morpholinopyridin-4-yl)-4-methylphenyl)-3-(2,2,2-trifluoroethyl)pyrrolidine-1-carboxamide formate

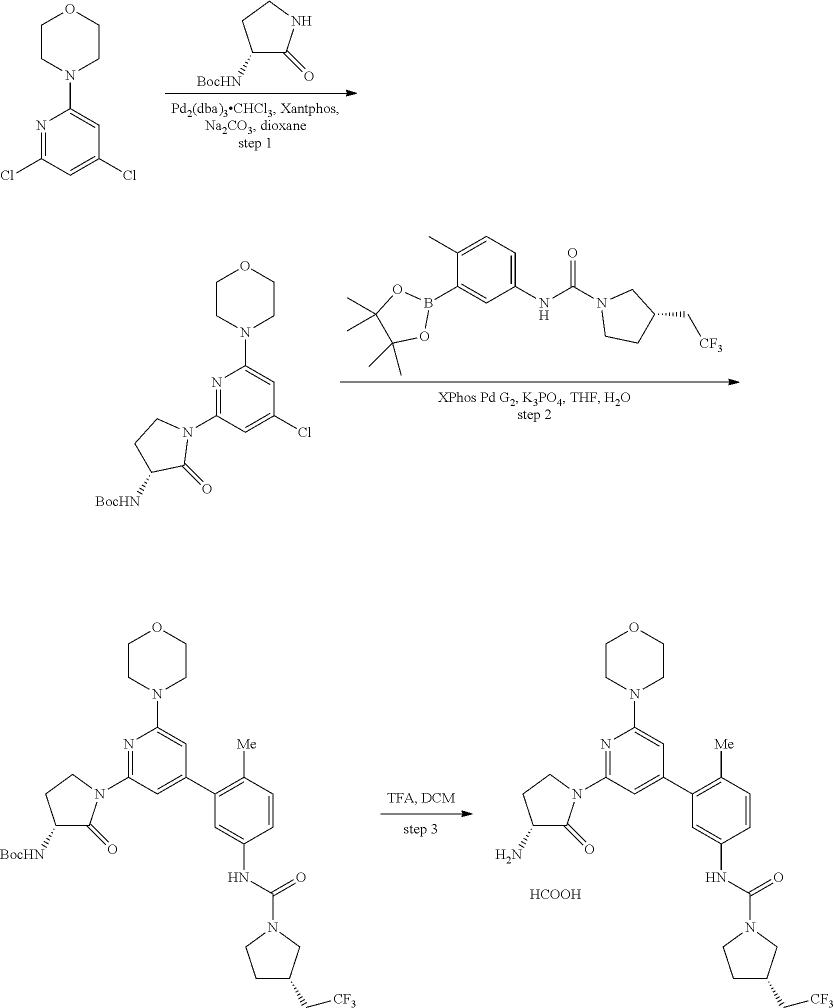

Preparation 132A: tert-butyl (R)-(1-(4-chloro-6-morpholinopyridin-2-yl)-2-oxopyrrolidin-3-yl)carbamate

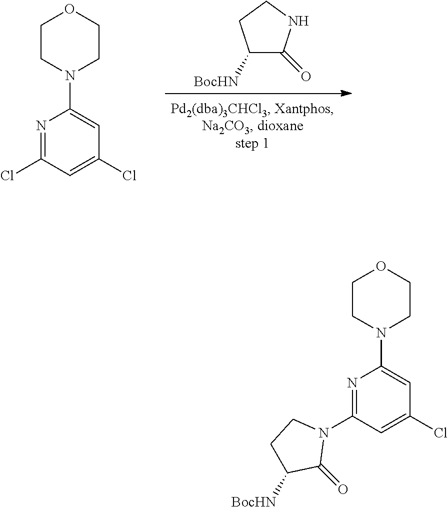 [SMILES: Clc1cc(Cl)nc(N2CCOCC2)c1 ; CC(C)(C)OC(=O)N[C@@H]1CCNC1=O ; CC(C)(C)OC(=O)N[C@@H]1CCN(c2cc(Cl)cc(N3CCOCC3)n2)C1=O]

A mixture of 4-(4,6-dichloropyridin-2-yl)morpholine (500 mg, 2.160 mmol) and tert-butyl (R)-(2-oxopyrrolidin-3-yl)carbamate (648 mg, 3.240 mmol), Pd₂(dba)₃·CHCl₃ (228 mg, 0.220 mmol), Xantphos (254 mg, 0.440 mmol) and Na₂CO₃ (687 mg, 6.480 mmol) in dioxane (5 mL) was stirred for 2 h at 100° C. under nitrogen atmosphere. The resulting mixture was concentrated under reduced pressure. The residue was purified by silica gel column chromatography, eluted with EtOAc/PE (1/4) to afford tert-butyl (R)-(1-(4-chloro-6-morpholinopyridin-2-yl)-2-oxopyrrolidin-3-yl)carbamate (680 mg, 80%) as a yellow solid. MS ESI calculated for C₁₈H₂₅ClN₄O₄[M+H]⁺, 397.16, found 397.05. ¹H NMR (400 MHz, Chloroform-d) δ 7.79 (s, 1H), 6.37 (s, 1H), 5.19 (s, 1H), 4.39-4.37 (m, 1H), 4.20-4.17 (m, 1H), 3.82-3.78 (m, 4H), 3.76-3.69 (m, 1H), 3.47-3.45 (m, 4H), 2.73-2.70 (m, 1H), 1.94-1.90 (m, 1H), 1.47 (s, 9H).

Preparation 132B: tert-butyl ((R)-1-(4-(2-methyl-5-((S)-3-(2,2,2-trifluoroethyl)pyrrolidine-1-carboxamido)phenyl)-6-morpholinopyridin-2-yl)-2-oxopyrrolidin-3-yl)carbamate

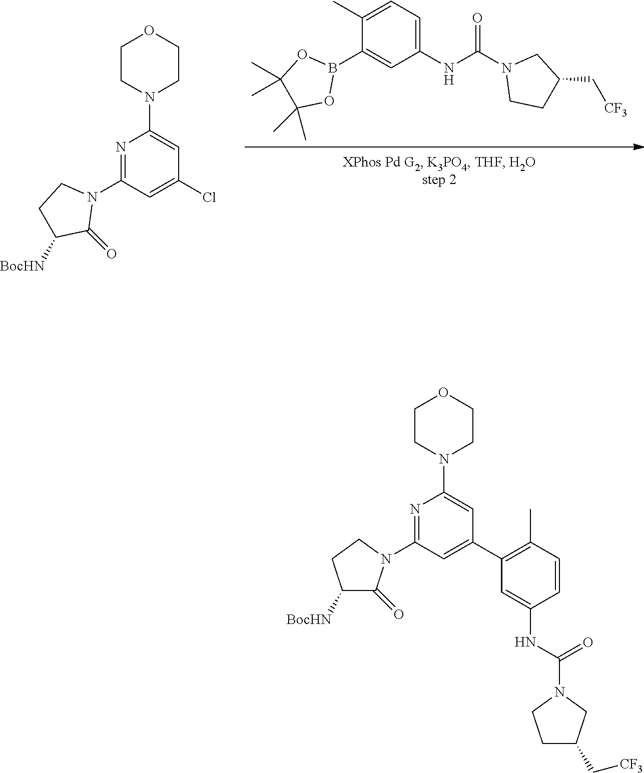 [SMILES: CC(C)(C)OC(=O)N[C@@H]1CCN(c2cc(Cl)cc(N3CCOCC3)n2)C1=O ; Cc1ccc(NC(=O)N2CC[C@@H](CC(F)(F)F)C2)cc1B1OC(C)(C)C(C)(C)O1 ; Cc1ccc(NC(=O)N2CC[C@@H](CC(F)(F)F)C2)cc1-c1cc(N2CCOCC2)nc(N2CC[C@@H](NC(=O)OC(C)(C)C)C2=O)c1]

A mixture of tert-butyl (R)-(1-(4-chloro-6-morpholinopyridin-2-yl)-2-oxopyrrolidin-3-yl)carbamate (200 mg, 0.505 mmol), (S)-N-(4-methyl-3-(4,4,5,5-tetramethyl-1,3,2-dioxaborolan-2-yl)phenyl)-3-(2,2,2-trifluoroethyl)pyrrolidine-1-carboxamide (250 mg, 0.606 mmol), 2^{nd} XPhos Precatalyst (40 mg, 0.051 mmol) and K₃PO₄ (212 mg, 1.010 mmol) in THF (5.0 mL) and H₂O (0.5 mL) was stirred for 3 h at 80° C. under nitrogen atmosphere. The resulting mixture was concentrated under reduced pressure. The residue was purified by silica gel column chromatography, eluted with EtOAc/PE (1/1) to afford tert-butyl ((R)-1-(4-(2-methyl-5-((S)-3-(2,2,2-trifluoroethyl)pyrrolidine-1-carboxamido)phenyl)-6-morpholinopyridin-2-yl)-2-oxopyrrolidin-3-yl)carbamate (309 mg, 96%) as a light yellow solid. MS ESI calculated for C₃₂H₄₁F₃N₆O₅[M+H]⁺, 647.31, found 647.20.

### Example 132: (S)-N-(3-(2-((R)-4-amino-2-oxopyrrolidin-1-yl)-6-morpholinopyridin-4-yl)-4-methylphenyl)-3-(2,2,2-trifluoroethyl)pyrrolidine-1-carboxamide formate

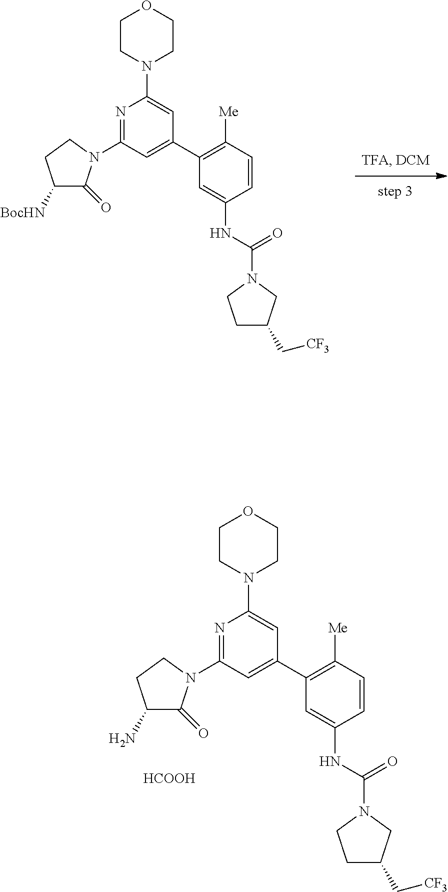 [SMILES: Cc1ccc(NC(=O)N2CC[C@@H](CC(F)(F)F)C2)cc1-c1cc(N2CCOCC2)nc(N2CC[C@@H](NC(=O)OC(C)(C)C)C2=O)c1 ; "HCOOH" ; Cc1ccc(NC(=O)N2CC[C@@H](CC(F)(F)F)C2)cc1-c1cc(N2CCOCC2)nc(N2CC[C@@H](N)C2=O)c1]

A mixture of tert-butyl ((R)-1-(4-(2-methyl-5-((S)-3-(2,2,2-trifluoroethyl)pyrrolidine-1-carboxamido)phenyl)-6-morpholinopyridin-2-yl)-2-oxopyrrolidin-3-yl)carbamate (200 mg, 0.309 mmol), DCM (5 mL) and TFA (0.5 mL) was stirred for 30 min at room temperature. The resulting mixture was concentrated under reduced pressure. The residue was purified by Prep-HPLC with following conditions: Column: Spherical C18, 20-40 μm, 120 g; Mobile Phase A: water (plus 10 mM FA); Mobile Phase B: CH₃CN; Flow rate: 60 mL/min; Gradient: 35% B to 65% B; Detector: 220 nm to afford (S)-N-(3-(2-((R)-4-amino-2-oxopyrrolidin-1-yl)-6-morpholinopyridin-4-yl)-4-methylphenyl)-3-(2,2,2-trifluoroethyl)pyrrolidine-1-carboxamide formate (100.2 mg, 59%) as a white solid. MS ESI calculated for C₂₈H₃₅F₃N₆O₅[M−HCOO]⁺, 547.26, found 547.30. ¹H NMR (400 MHz, Chloroform-d) δ 8.18 (s, 1H), 7.68 (s, 1H), 7.36 (dd, J=8.4, 2.4 Hz, 1H), 7.22 (d, J=2.4 Hz, 1H), 7.14 (d, J=8.4 Hz, 1H), 6.47 (s, 1H), 6.34 (s, 1H), 4.26-4.15 (m, 1H), 3.95-3.68 (m, 7H), 3.65-3.56 (m, 1H), 3.49-3.46 (m, 4H), 3.42-3.20 (m, 3H), 3.09-3.06 (m, 1H), 2.55-2.50 (m, 2H), 2.32-2.10 (m, 6H), 1.97-1.86 (m, 1H), 1.73-1.61 (m, 1H). ¹⁹F NMR (376 MHz, Chloroform-d₆) δ-64.92 (3F).

### Examples 133 and 134: (3S)-N-(3-{2-[(4R)-4-amino-3,3-difluoropyrrolidin-1-yl]-6-(morpholin-4-yl)pyridin-4-yl}-4-methylphenyl)-3-(2,2,2-trifluoroethyl)pyrrolidine-1-carboxamide and (3S)-N-(3-{2-[(4S)-4-amino-3,3-difluoropyrrolidin-1-yl]-6-(morpholin-4-yl)pyridin-4-yl}-4-methylphenyl)-3-(2,2,2-trifluoroethyl)pyrrolidine-1-carboxamide

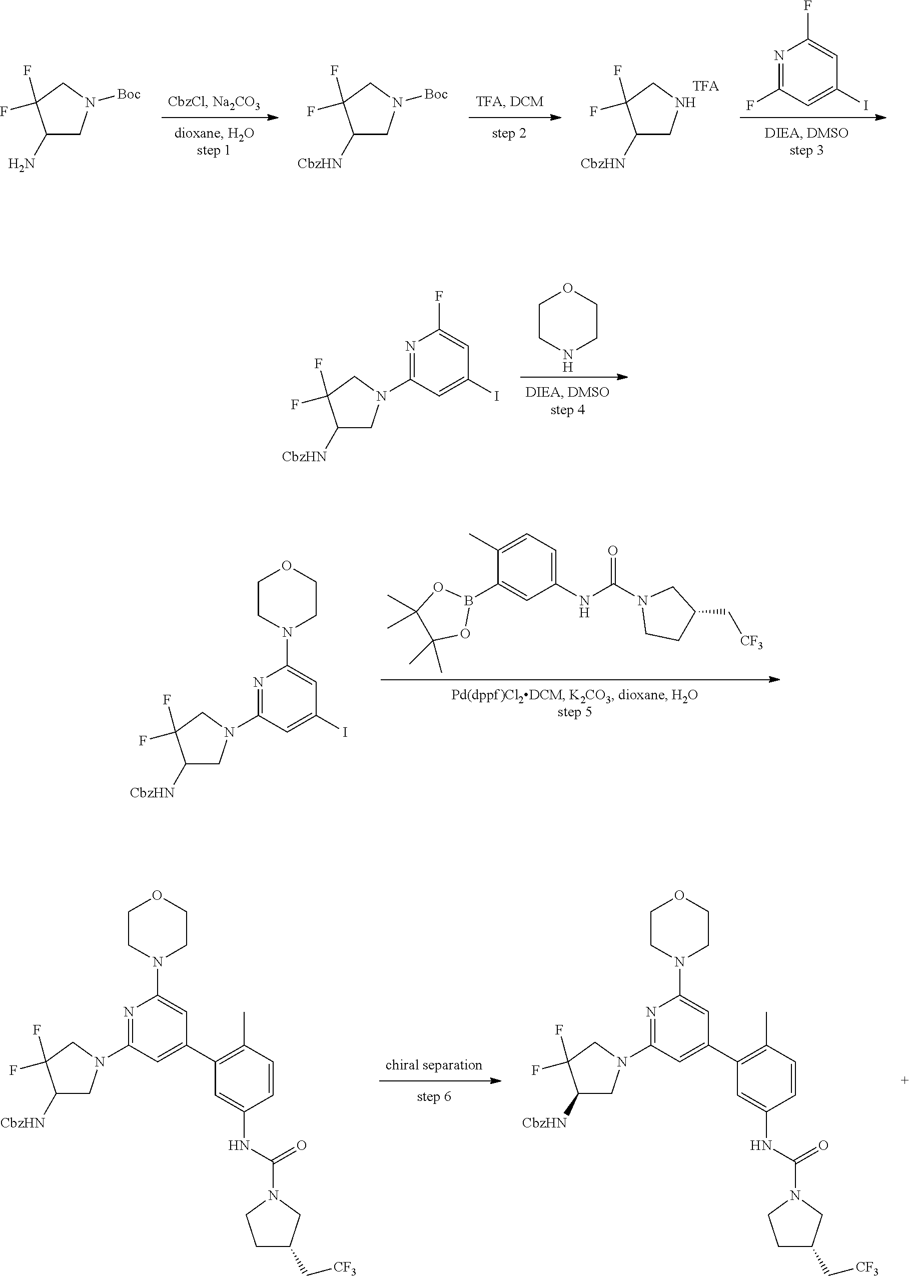

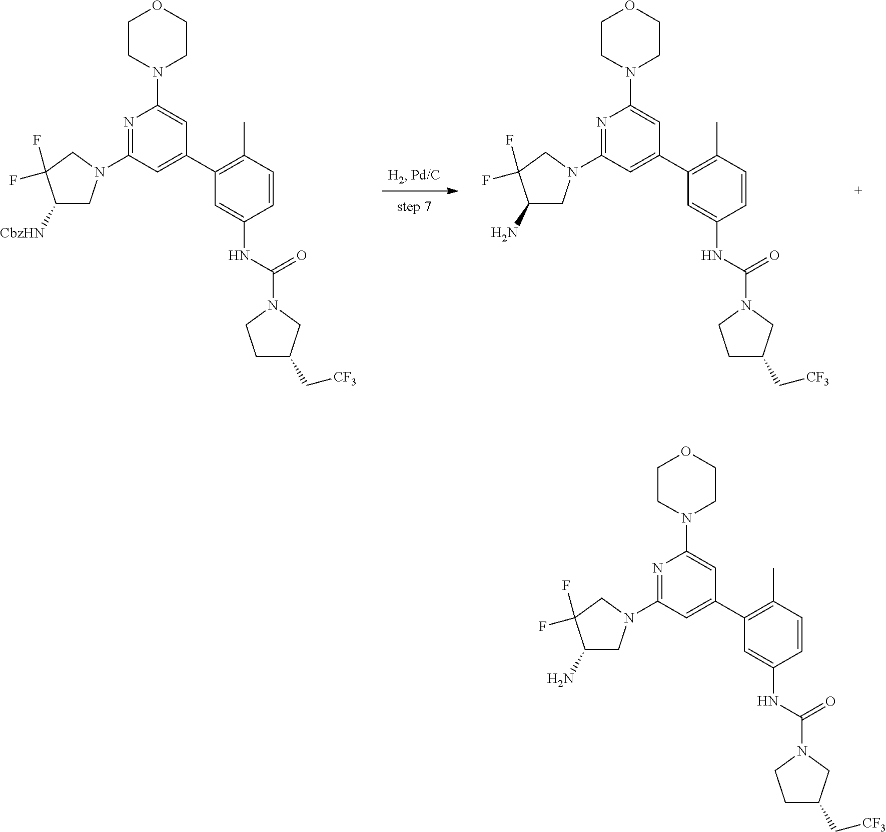 [SMILES: Cc1ccc(NC(=O)N2CC[C@@H](CC(F)(F)F)C2)cc1-c1cc(N2CCOCC2)nc(N2C[C@H](NC(=O)OCc3ccccc3)C(F)(F)C2)c1 ; Cc1ccc(NC(=O)N2CC[C@@H](CC(F)(F)F)C2)cc1-c1cc(N2CCOCC2)nc(N2C[C@@H](N)C(F)(F)C2)c1 ; Cc1ccc(NC(=O)N2CC[C@@H](CC(F)(F)F)C2)cc1-c1cc(N2CCOCC2)nc(N2C[C@H](N)C(F)(F)C2)c1]

Preparation 133A: tert-butyl 4-{[(benzyloxy)carbonyl]amino}-3,3-difluoropyrrolidine-1-carboxylate

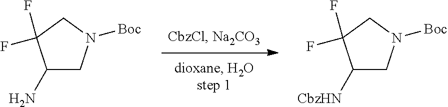 [SMILES: CC(C)(C)OC(=O)N1CC(N)C(F)(F)C1 ; CC(C)(C)OC(=O)N1CC(NC(=O)OCc2ccccc2)C(F)(F)C1]

To a stirred solution of tert-butyl 4-amino-3,3-difluoropyrrolidine-1-carboxylate (200 mg, 0.900 mmol) and Na₂CO₃ (114 mg, 1.080 mmol) in 1,4-dioxane (2 mL) and H₂O (2 mL) was added Cbz-Cl (184 mg, 1.080 mmol) at 0° C. The resulting mixture was stirred for 4 h at room temperature. The reaction mixture was diluted with water (30 mL). The resulting mixture was extracted with EtOAc (3×30 mL). The combined organic layers were washed with brine (30 mL), dried over anhydrous Na₂SO₄. After filtration, the filtrate was concentrated under reduced pressure to afford tert-butyl 4-{[(benzyloxy)carbonyl]amino}-3,3-difluoropyrrolidine-1-carboxylate (700 mg, crude) as yellow oil. MS ESI calculated for C₁₇H₂₂F₂N₂O₄[M-Boc+H]⁺, 257.15; found 257.10.

Preparation 133B: benzyl N-(4,4-difluoropyrrolidin-3-yl)carbamate 2,2,2-trifluoroacetate

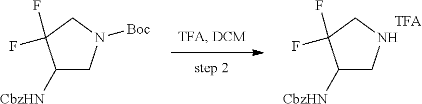 [SMILES: CC(C)(C)OC(=O)N1CC(NC(=O)OCc2ccccc2)C(F)(F)C1 ; O=C(NC1CNCC1(F)F)OCc1ccccc1]

To a stirred solution of tert-butyl 4-{[(benzyloxy)carbonyl]amino}-3,3-difluoropyrrolidine-1-carboxylate (700 mg, 1.964 mmol) in DCM (10 mL) was added TFA (2 mL) dropwise at 0° C. The resulting mixture was stirred for 2 h at room temperature. The resulting mixture was concentrated under reduced pressure to afford benzyl N-(4,4-difluoropyrrolidin-3-yl)carbamate 2,2,2-trifluoroacetate (750 mg, crude) as yellow oil. MS ESI calculated for C₁₄H₁₅F₅N₂O₄[M—CF₃COO]⁺, 257.10, found 257.10.

Preparation 133C: 6-fluoro-4-iodo-N-(4-methyloxan-4-yl)pyridin-2-amine

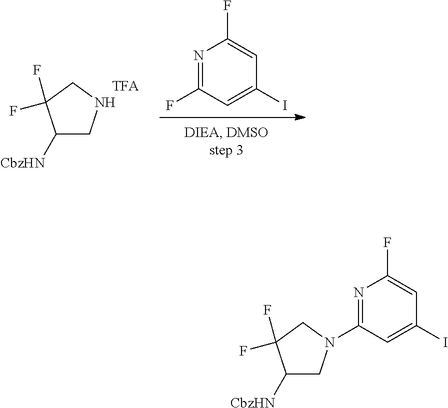 [SMILES: O=C(NC1CNCC1(F)F)OCc1ccccc1 ; Fc1cc(I)cc(F)n1 ; O=C(NC1CN(c2cc(I)cc(F)n2)CC1(F)F)OCc1ccccc1]

A solution of 2,6-difluoro-4-iodopyridine (700 mg, 2.905 mmol), benzyl N-(4,4-difluoropyrrolidin-3-yl)carbamate 2,2,2-trifluoroacetate (893 mg, 3.486 mmol) in DMSO (7 mL) and DIEA (825 mg, 6.391 mmol) was stirred for 4 h at 80° C. The resulting mixture was diluted with H₂O (50 mL) and extracted with EtOAc (3×50 mL). The combined organic layers were washed with brine (15 mL), dried over anhydrous Na₂SO₄. After filtration, the filtrate was concentrated under reduced pressure. The residue was purified by silica gel column chromatography, eluted with PE/EtOAc (4/1) to afford 6-fluoro-4-iodo-N-(4-methyloxan-4-yl)pyridin-2-amine (550 mg, 56%) as a yellow solid. MS ESI calculated for C₁₇H₁₅F₃₁N₃O₂[M+H]⁺, 478.02, found 477.90.

Preparation 133D: benzyl N-[4,4-difluoro-1-[4-iodo-6-(morpholin-4-yl)pyridin-2-yl]pyrrolidin-3-yl]carbamate

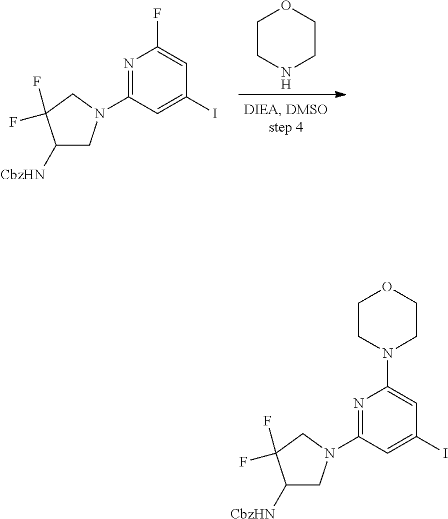 [SMILES: O=C(NC1CN(c2cc(I)cc(F)n2)CC1(F)F)OCc1ccccc1 ; C1COCCN1 ; O=C(NC1CN(c2cc(I)cc(N3CCOCC3)n2)CC1(F)F)OCc1ccccc1]

To a stirred solution of benzyl N-[4,4-difluoro-1-(6-fluoro-4-iodopyridin-2-yl)pyrrolidin-3-yl]carbamate (500 mg, 1.048 mmol) and morpholine (109 mg, 1.257 mmol) in DMSO (5.00 mL) was added DIEA (203 mg, 1.572 mmol) at room temperature. The resulting mixture was stirred for 16 h at 80° C. The reaction was quenched with water (50 mL). The resulting mixture was extracted with EtOAc (3×50 mL). The combined organic layers were washed with brine (2×30 mL), dried over anhydrous Na₂SO₄. After filtration, the filtrate was concentrated under reduced pressure. The residue was purified by silica gel column chromatography, eluted with PE/EA (2/1) to afford benzyl N-[4,4-difluoro-1-[4-iodo-6-(morpholin-4-yl)pyridin-2-yl]pyrrolidin-3-yl]carbamate (480 mg, 84%) as a white solid. MS ESI calculated for C₂₁H₂₃F₂IN₄O₃[M+H]⁺, 545.08, found 545.10. ¹H NMR (400 MHz, Chloroform-d) δ 7.40-7.30 (m, 5H), 6.36 (s, 1H), 6.10 (s, 1H), 5.15 (s, 2H), 4.66-4.62 (m, 1H), 4.04-3.98 (m, 1H), 3.90-3.67 (m, 7H), 3.49-3.40 (m, 4H), 3.24-3.20 (m, 1H).

Preparation 133E: benzyl N-[4,4-difluoro-1-(4-{2-methyl-5-[(3S)-3-(2,2,2-trifluoroethyl)pyrrolidine-1-carbonylamino]phenyl}-6-(morpholin-4-yl)pyridin-2-yl)pyrrolidin-3-yl]carbamate

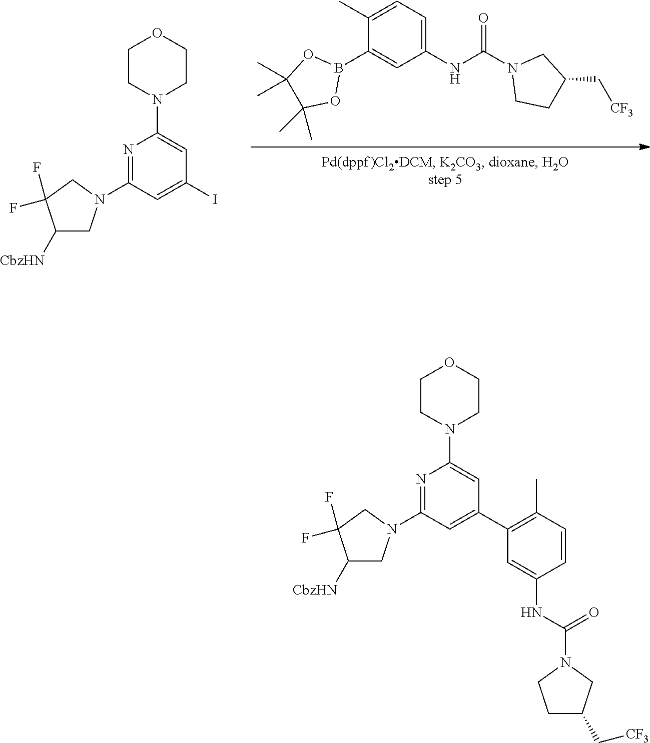 [SMILES: O=C(NC1CN(c2cc(I)cc(N3CCOCC3)n2)CC1(F)F)OCc1ccccc1 ; Cc1ccc(NC(=O)N2CC[C@@H](CC(F)(F)F)C2)cc1B1OC(C)(C)C(C)(C)O1 ; Cc1ccc(NC(=O)N2CC[C@@H](CC(F)(F)F)C2)cc1-c1cc(N2CCOCC2)nc(N2CC(NC(=O)OCc3ccccc3)C(F)(F)C2)c1]

A mixture of benzyl N-{4,4-difluoro-1-[4-iodo-6-(morpholin-4-yl)pyridin-2-yl]pyrrolidin-3-yl}carbamate (300 mg, 0.551 mmol), N-[4-methyl-3-(4,4,5,5-tetramethyl-1,3,2-dioxaborolan-2-yl)phenyl]-2-(trifluoromethyl)pyridine-4-carboxamide (223 mg, 0.551 mmol), 1,4-dioxane (3 mL), H₂O (0.7 mL), Pd(dppf)Cl₂·CH₂Cl₂ (45 mg, 0.055 mmol) and K₂CO₃ (152 mg, 1.101 mmol) was stirred for 2 h at 80° C. under nitrogen atmosphere. The reaction was quenched with water (50 mL). The resulting mixture was extracted with EtOAc (3×50 mL). The combined organic layers were washed with brine (2×30 mL), dried over anhydrous Na₂SO₄. After filtration, the filtrate was concentrated under reduced pressure. The residue was purified by silica gel column chromatography, eluted with PE/EtOAc/EtOH (2/3/1) to afford benzyl N-[4,4-difluoro-1-(4-{2-methyl-5-[(3S)-3-(2,2,2-trifluoroethyl)pyrrolidine-1-carbonylamino]phenyl}-6-(morpholin-4-yl)pyridin-2-yl)pyrrolidin-3-yl]carbamate (300 mg, 77%) as yellow oil. MS ESI calculated for C₃₅H₃₉F₅N₆O₄[M+H]⁺,703.30, found 703.15. ¹H NMR (400 MHz, Chloroform-d) δ 7.42-7.35 (m, 6H), 7.23 (d, J=2.4 Hz, 1H), 7.18 (d, J=8.4 Hz, 1H), 6.14 (s, 1H), 5.99 (s, 1H), 5.71 (s, 1H), 5.21-5.17 (m, 3H), 4.74-4.68 (m, 1H), 4.12-4.07 (m, 1H), 3.98-3.77 (m, 7H), 3.64 (t, J=8.8 Hz, 1H), 3.50-3.42 (m, 5H), 3.31 (t, J=9.2 Hz, 1H), 3.12 (t, J=9.2 Hz, 1H), 2.56-2.53 (m, 1H), 2.31-2.20 (m, 5H), 1.77-1.71 (m, 1H).

Preparations 133F/134F: Benzyl N-[(3R)-4,4-difluoro-1-(4-{2-methyl-5-[(3S)-3-(2,2,2-trifluoroethyl)pyrrolidine-1-carbonylamino]phenyl}-6-(morpholin-4-yl)pyridin-2-yl)pyrrolidin-3-yl]carbamate and benzyl N-[(3S)-4,4-difluoro-1-(4-{2-methyl-5-[(3S)-3-(2,2,2-trifluoroethyl)pyrrolidine-1-carbonylamino]phenyl}-6-(morpholin-4-yl)pyridin-2-yl)pyrrolidin-3-yl]carbamate

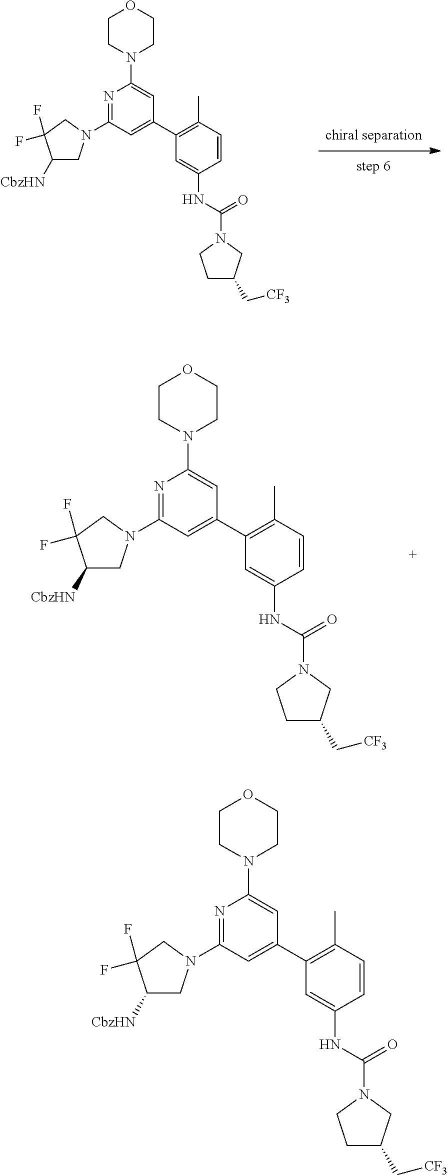 [SMILES: Cc1ccc(NC(=O)N2CC[C@@H](CC(F)(F)F)C2)cc1-c1cc(N2CCOCC2)nc(N2CC(NC(=O)OCc3ccccc3)C(F)(F)C2)c1 ; Cc1ccc(NC(=O)N2CC[C@@H](CC(F)(F)F)C2)cc1-c1cc(N2CCOCC2)nc(N2C[C@@H](NC(=O)OCc3ccccc3)C(F)(F)C2)c1 ; Cc1ccc(NC(=O)N2CC[C@@H](CC(F)(F)F)C2)cc1-c1cc(N2CCOCC2)nc(N2C[C@H](NC(=O)OCc3ccccc3)C(F)(F)C2)c1]

Benzyl N-[4,4-difluoro-1-(4-{2-methyl-5-[(3S)-3-(2,2,2-trifluoroethyl)pyrrolidine-1-carbonylamino]phenyl}-6-(morpholin-4-yl)pyridin-2-yl)pyrrolidin-3-yl]carbamate (330 mg, 0.470 mmol) was resolved by chiral Prep-HPLC with the following conditions: Column: CHIRALPAK IE, 2*25 cm, 5 μm; Mobile Phase A: Hexane (0.5% 2M NH₃-MeOH), Mobile Phase B: EtOH; Flow rate:20 mL/min; Gradient: A:B=70:30; 220/254 nm. RT₁: 16.88 min gave 100 mg (30%) of Preparation 133F as a white solid. MS ESI calculated for C₃₅H₃₉F₅N₆O₄[M+H]⁺, 703.30, found 703.35. And RT₂: 22.22 min gave 110 mg (33%) of Preparation 134F as a white solid. MS ESI calculated for C₃₅H₃₉F₅N₆O₄[M+H]⁺, 703.30, found 703.40.

### Examples 133 and 134: (3S)-N-(3-{2-[(4R)-4-amino-3,3-difluoropyrrolidin-1-yl]-6-(morpholin-4-yl)pyridin-4-yl}-4-methylphenyl)-3-(2,2,2-trifluoroethyl)pyrrolidine-1-carboxamide and (3S)-N-(3-{2-[(4S)-4-amino-3,3-difluoropyrrolidin-1-yl]-6-(morpholin-4-yl)pyridin-4-yl}-4-methylphenyl)-3-(2,2,2-trifluoroethyl)pyrrolidine-1- carboxamide

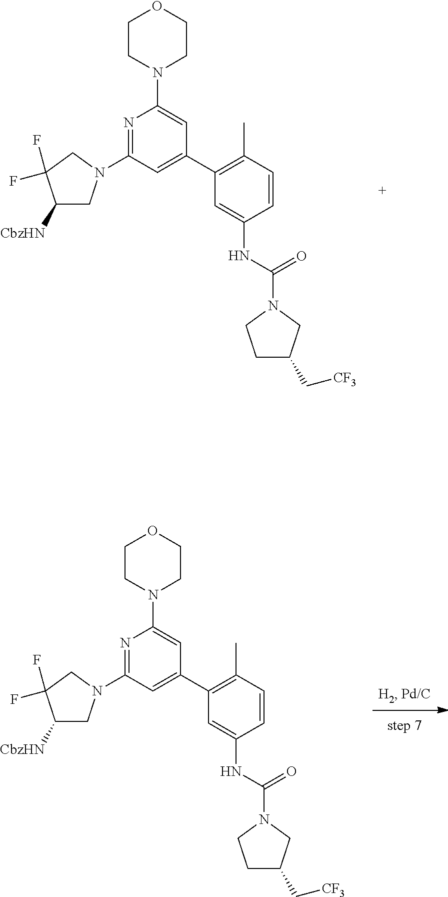 [SMILES: Cc1ccc(NC(=O)N2CC[C@@H](CC(F)(F)F)C2)cc1-c1cc(N2CCOCC2)nc(N2C[C@@H](NC(=O)OCc3ccccc3)C(F)(F)C2)c1 ; Cc1ccc(NC(=O)N2CC[C@@H](CC(F)(F)F)C2)cc1-c1cc(N2CCOCC2)nc(N2C[C@H](NC(=O)OCc3ccccc3)C(F)(F)C2)c1]

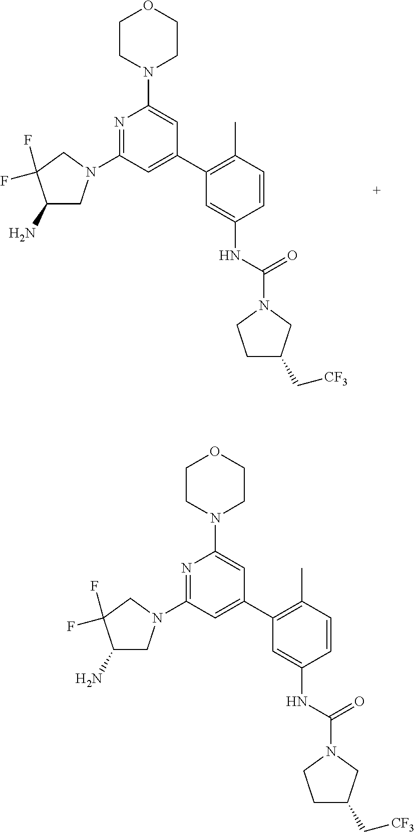 [SMILES: Cc1ccc(NC(=O)N2CC[C@@H](CC(F)(F)F)C2)cc1-c1cc(N2CCOCC2)nc(N2C[C@@H](N)C(F)(F)C2)c1 ; Cc1ccc(NC(=O)N2CC[C@@H](CC(F)(F)F)C2)cc1-c1cc(N2CCOCC2)nc(N2C[C@H](N)C(F)(F)C2)c1]

A mixture of Preparation 133F (110 mg, 0.157 mmol) in MeOH (3 mL) and Pd/C (50 mg) was stirred for 30 min at room temperature under hydrogen atmosphere. The resulting mixture was filtered and washed with MeOH (3×10 mL). The filtrate was concentrated under reduced pressure to afford Example 133 (70 mg, 78%) as a white solid. MS ESI calculated for C₂₇H3₃F₅N₆O₂[M+H]⁺, 569.26, found 569.25. ¹H NMR (400 MHz, DMSO-d₆) δ 8.12 (s, 1H), 7.43 (dd, J=2.4, 8.4 Hz, 1H), 7.33 (d, J=2.4 Hz, 1H), 7.11 (d, J=8.4 Hz, 1H), 5.95 (s, 1H), 5.70 (s, 1H), 3.90-3.73 (m, 3H), 3.72-3.63 (m, 6H), 3.52 (t, J=9.2 Hz, 1H), 3.42-3.40 (m, 4H), 3.31-3.29 (m, 1H) 3.15 (t, J=8.8 Hz, 1H), 3.02 (t, J=9.6 Hz, 1H), 2.45-2.52 (m, 3H), 2.17 (s, 3H), 2.10-2.07 (m, 1H), 1.94 (s, 1H), 1.70-1.62 (m, 1H). ¹⁹F NMR (376 MHz, DMSO-d₆) δ-66.37 (3F), −110.15 (d, J=224.8 Hz, 1F), −117.55 (d, J=224.8 Hz, 1F).

A mixture of Preparation 134F (110 mg, 0.157 mmol), MeOH (3 mL) and Pd/C (50 mg) was stirred for 30 min at room temperature under hydrogen atmosphere. The resulting mixture was filtered and washed with MeOH (3×10 mL). The resulting mixture was concentrated under reduced pressure to afford Example 134 (72 mg, 88%) as a white solid. MS ESI calculated for C₂₇H3₃F₅N₆O₂[M+H]⁺, 569.26, found 569.20. ¹H NMR (400 MHz, DMSO-d₆) δ 8.12 (s, 1H), 7.43 (dd, J=2.4, 8.4 Hz, 1H), 7.34 (d, J=2.4 Hz, 1H), 7.11 (d, J=8.4 Hz, 1H), 5.95 (s, 1H), 5.70 (s, 1H), 3.90-3.73 (m, 3H), 3.72-3.63 (m, 6H), 3.52 (t, J=9.2 Hz, 1H), 3.42-3.40 (m, 4H), 3.31-3.29 (m, 1H) 3.15 (t, J=8.8 Hz, 1H), 3.02 (t, J=9.6 Hz, 1H), 2.45-2.52 (m, 3H), 2.17 (s, 3H), 2.10-2.05 (m, 1H), 1.94 (s, 1H), 1.69-1.63 (m, 1H). ¹⁹F NMR (376 MHz, DMSO-d₆) δ-63.36 (3F), −110.13 (d, J=224.8 Hz, 1F), −117.54 (d, J=224.8 Hz, 1F).

### Examples 135 and 136: (3S)-N-(3-{2-[(3R,4R)-3-amino-4-hydroxypyrrolidin-1-yl]-6-(morpholin-4-yl)pyridin-4-yl}-4-methylphenyl)-3-(2,2,2-trifluoroethyl)pyrrolidine-1-carboxamide and (3S)-N-(3-{2-[(3S,4S)-3-amino-4-hydroxypyrrolidin-1-yl]-6-(morpholin-4-yl)pyridin-4-yl}-4-methylphenyl)-3-(2,2,2-trifluroethyl)pyrrolidine-1- carboxamide

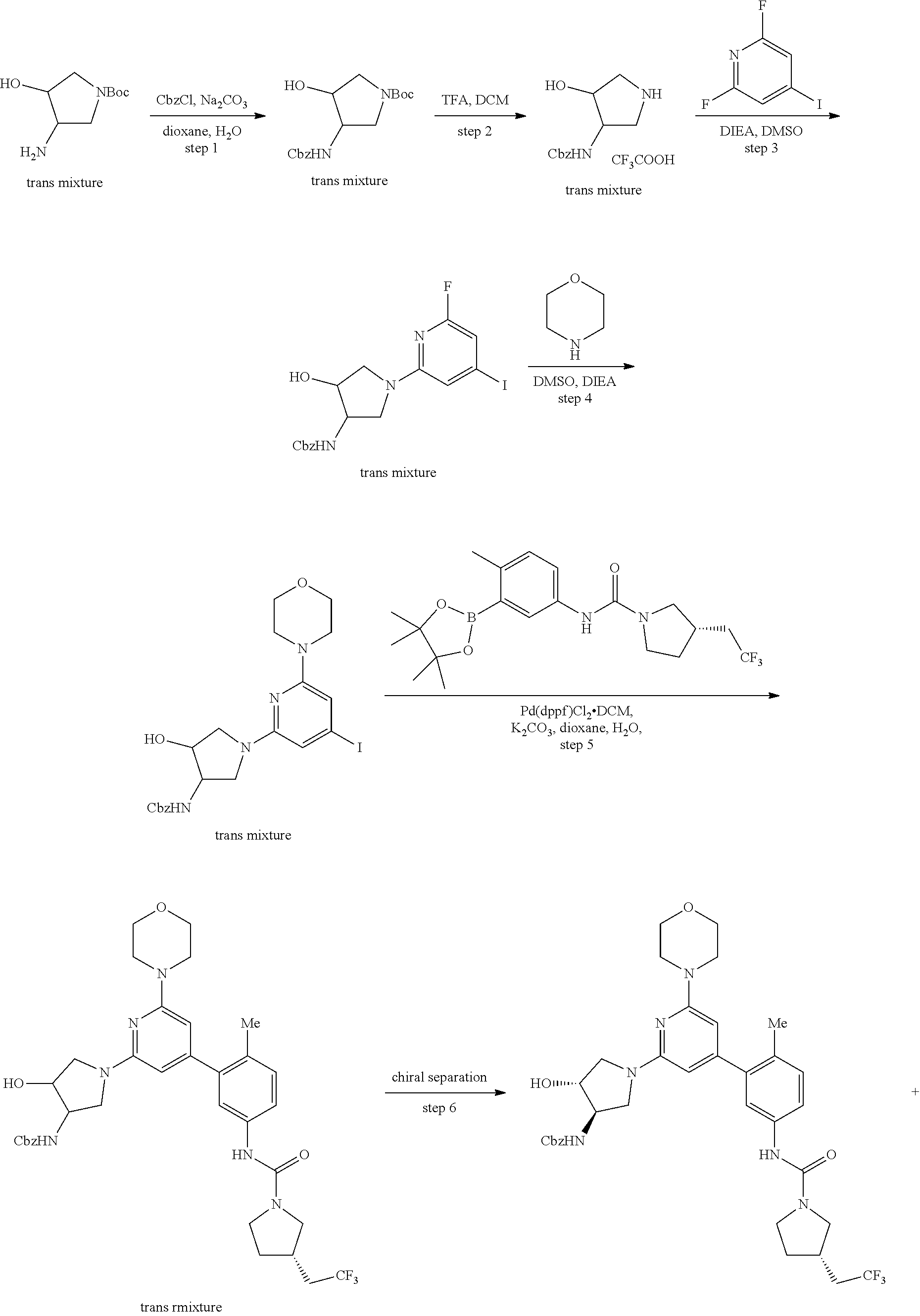

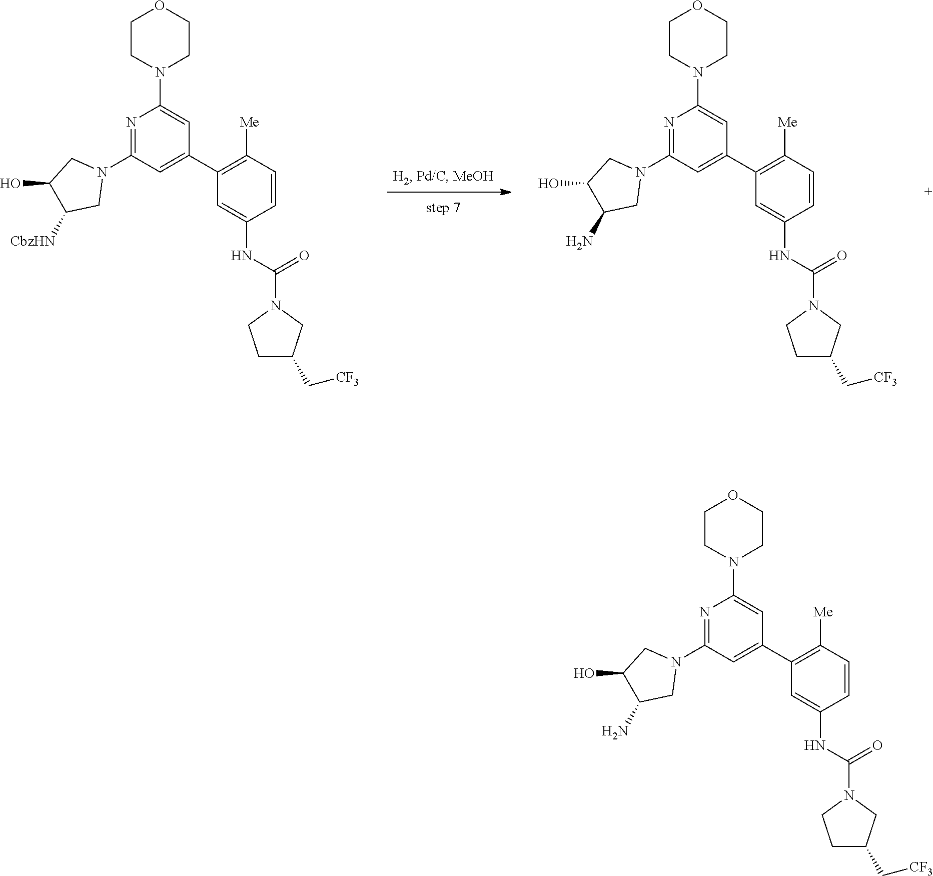 [SMILES: Cc1ccc(NC(=O)N2CC[C@@H](CC(F)(F)F)C2)cc1-c1cc(N2CCOCC2)nc(N2C[C@H](NC(=O)OCc3ccccc3)[C@@H](O)C2)c1 ; Cc1ccc(NC(=O)N2CC[C@@H](CC(F)(F)F)C2)cc1-c1cc(N2CCOCC2)nc(N2C[C@@H](N)[C@H](O)C2)c1 ; Cc1ccc(NC(=O)N2CC[C@@H](CC(F)(F)F)C2)cc1-c1cc(N2CCOCC2)nc(N2C[C@H](N)[C@@H](O)C2)c1]

Preparation 135A: Trans-tert-butyl-3-[[(benzyloxy)carbonyl]amino]-4-hydroxypyrrolidine-1-carboxylate

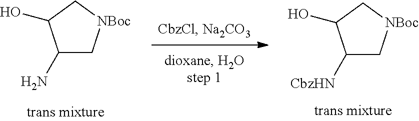 [SMILES: CC(C)(C)OC(=O)N1CC(N)C(O)C1 ; CC(C)(C)OC(=O)N1CC(O)C(NC(=O)OCc2ccccc2)C1]

To a stirred mixture of trans-tert-butyl-3-amino-4-hydroxypyrrolidine-1-carboxylate (5 g, 24.721 mmol) and Na₂CO₃ (3 g, 29.666 mmol) in dioxane (57 mL) and H₂O (57 mL) was added Cbz-Cl (5 g, 29.661 mmol) dropwise at 0° C. The resulting mixture was stirred for 3 h at room temperature. The reaction mixture was diluted with water (100 mL). The resulting mixture was extracted with EtOAc (2×200 mL). The combined organic layers were washed with brine (50 mL), dried over anhydrous Na₂SO₄. After filtration, the filtrate was concentrated under reduced pressure. The residue was purified by silica gel column chromatography, eluted with PE/EtOAc/EtOH (4/3/1) to afford trans-tert-butyl-3-[[(benzyloxy)carbonyl]amino]-4-hydroxypyrrolidine-1-carboxylate (7.2 g, 78%) as an off-white solid. MS ESI calculated for C₁₇H24N₂O₅ [M-Boc+H]⁺, 237.17, found 237.10. ¹H NMR (300 MHz, Chloroform-d) δ 7.40-7.33 (m, 5H), 5.11 (s, 2H), 4.27-4.23 (m, 1H), 4.02-3.98 (m, 1H), 3.79 (m, 1H), 3.65 (m, 1H), 3.33-3.24 (m, 2H), 1.46 (s, 9H).

Preparation 135B: Trans-benzyl (4-hydroxypyrrolidin-3-yl)carbamate 2,2,2-trifluoroacetate

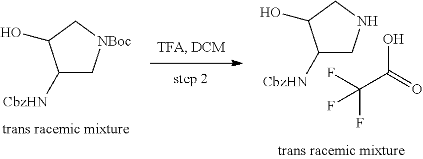

To a stirred mixture of trans-tert-butyl-3-[[(benzyloxy)carbonyl]amino]-4-hydroxypyrrolidine-1-carboxylate (2 g, 5.946 mmol) in DCM (20 mL) was added TFA (4 mL, 53.852 mmol) dropwise at 0° C. The resulting mixture was stirred for 30 min at room temperature. The resulting mixture was concentrated under reduced pressure to afford trans-benzyl (4-hydroxypyrrolidin-3-yl)carbamate 2,2,2-trifluoroacetate (3 g, crude) as a light brown oil. MS ESI calculated for C₁₄H₁₇F₃N₂O₅[M-CF₃COO]⁺, 237.12, found 237.10.

Preparation 135C: Trans-benzyl[1-(6-fluoro-4-iodopyridin-2-yl)-4-hydroxypyrrolidin-3-yl]carbamate

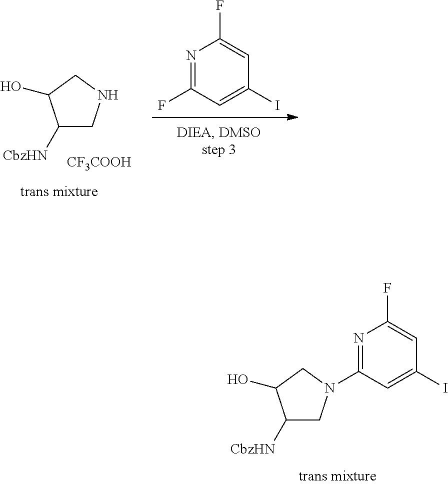 [SMILES: O=C(NC1CNCC1O)OCc1ccccc1 ; "CF3COOH" ; Fc1cc(I)cc(F)n1 ; O=C(NC1CN(c2cc(I)cc(F)n2)CC1O)OCc1ccccc1]

A mixture of 2,6-difluoro-4-iodopyridine (1.5 g, 6.225 mmol), trans-benzyl (4-hydroxypyrrolidin-3-yl)carbamate 2,2,2-trifluoroacetate (2.2 g, 6.223 mmol) and DIEA (4 g, 31.104 mmol) in DMSO (15 mL) was stirred for 16 h at 80° C. under nitrogen atmosphere. The reaction was quenched with water (80 mL). The resulting mixture was extracted with EtOAc (2×30 mL). The combined organic layers were washed with brine (10 mL), dried over anhydrous Na₂SO₄. After filtration, the filtrate was concentrated under reduced pressure. The residue was purified by silica gel column chromatography, eluted with PE/EtOAc (1/1) to afford trans-benzyl[1-(6-fluoro-4-iodopyridin-2-yl)-4-hydroxypyrrolidin-3-yl]carbamate (2.1 g, 66%) as an off-white solid. MS ESI calculated for C₁₇H₁₇FIN₃O₃[M+H]⁺, 458.03, found 458.10. ¹H NMR (300 MHz, Chloroform-d) δ 7.40-7.36 (m, 5H), 6.58-6.56 (m, 2H), 5.15 (s, 2H), 4.95-4.91 (m, 1H), 4.45-4.43 (m, 1H), 4.18-4.11 (m, 1H), 3.96-3.90 (m, 1H), 3.79-3.74 (m, 1H), 3.43-3.35 (m, 2H).

Preparation 135D: Trans-benzyl (4-hydroxy-1-(4-iodo-6-morpholinopyridin-2-yl)pyrrolidin-3-yl)carbamate

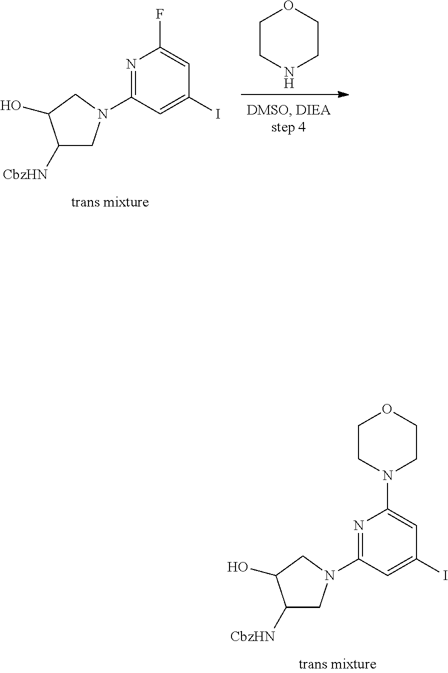 [SMILES: O=C(NC1CN(c2cc(I)cc(F)n2)CC1O)OCc1ccccc1 ; C1COCCN1 ; O=C(NC1CN(c2cc(I)cc(N3CCOCC3)n2)CC1O)OCc1ccccc1]

A mixture of trans-benzyl[1-(6-fluoro-4-iodopyridin-2-yl)-4-hydroxypyrrolidin-3-yl]carbamate (1 g, 2.187 mmol), morpholine (1.9 g, 21.914 mmol) and DIEA (0.85 g, 6.577 mmol) in DMSO (10 mL) was stirred for 16 h at 80° C. under nitrogen atmosphere. The mixture was quenched with water (50 mL). The resulting mixture was extracted with EtOAc (2×30 mL). The combined organic layers were washed with brine (10 mL), dried over anhydrous Na₂SO₄. After filtration, the filtrate was concentrated under reduced pressure. The residue was triturated in ethyl acetate (20 mL) to afford trans-benzyl (4-hydroxy-1-(4-iodo-6-morpholinopyridin-2-yl)pyrrolidin-3-yl)carbamate (760 mg, 60%) as an off-white solid. MS ESI calculated for C₂₁H₂₅IN₄O₄[M+H]⁺, 525.09, found 525.25. ¹H NMR (400 MHz, DMSO-d₆) δ 7.39-7.30 (m, 5H), 6.32 (s, 1H), 6.09 (s, 1H), 5.03 (s, 2H), 4.10-4.04 (m, 1H), 3.90-3.87 (m, 1H), 3.65-3.58 (m, 4H), 3.57-3.52 (m, 1H), 3.37-3.35 (m, 5H), 3.25-3.17 (m, 2H).

Preparation 135E: Trans-benzyl (4-hydroxy-1-(4-(2-methyl-5-((S)-3-(2,2,2-trifluoroethyl)pyrrolidine-1-carboxamido)phenyl)-6-morpholinopyridin-2-yl)pyrrolidin-3-yl)carbamate

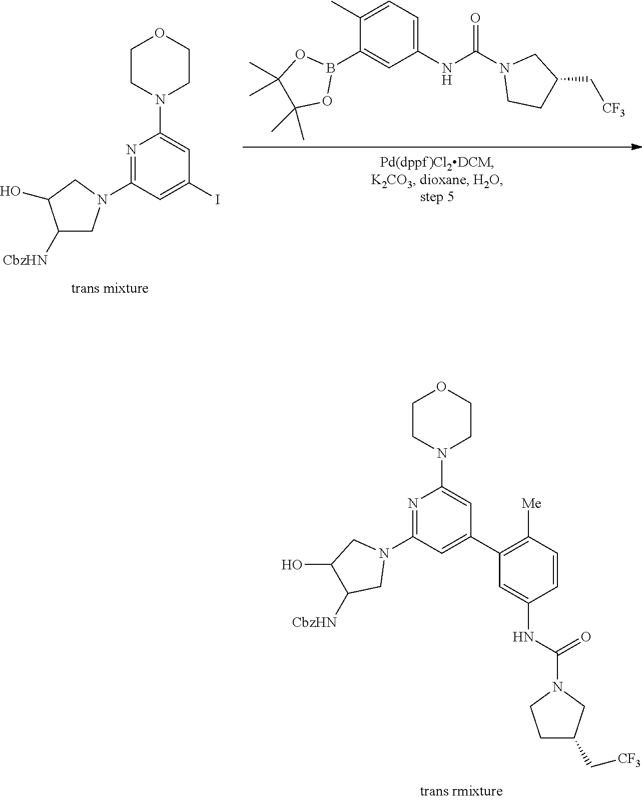 [SMILES: O=C(NC1CN(c2cc(I)cc(N3CCOCC3)n2)CC1O)OCc1ccccc1 ; Cc1ccc(NC(=O)N2CC[C@@H](CC(F)(F)F)C2)cc1B1OC(C)(C)C(C)(C)O1 ; Cc1ccc(NC(=O)N2CC[C@@H](CC(F)(F)F)C2)cc1-c1cc(N2CCOCC2)nc(N2CC(O)C(NC(=O)OCc3ccccc3)C2)c1]

A mixture of trans-benzyl (4-hydroxy-1-(4-iodo-6-morpholinopyridin-2-yl)pyrrolidin-3-yl)carbamate (300 mg, 0.572 mmol), (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (236 mg, 0.572 mmol), K₂CO₃ (237 mg, 1.716 mmol) and Pd(dppf)Cl₂·CH₂Cl₂ (47 mg, 0.057 mmol) in dioxane (3 mL) and H₂O (0.75 mL) was stirred for 16 h at 80° C. under nitrogen atmosphere. The resulting mixture was concentrated under reduced pressure. The residue was purified by silica gel column chromatography, eluted with PE/EtOAc/EtOH (8/3/1) to afford trans-benzyl (4-hydroxy-1-(4-(2-methyl-5-((S)-3-(2,2,2-trifluoroethyl)pyrrolidine-1-carboxamido)phenyl)-6-morpholinopyridin-2-yl)pyrrolidin-3-yl)carbamate (270 mg, 66%) as an off-white solid. MS ESI calculated for C₃₅H₄₁F₃N₆O₅[M+H]⁺, 683.31, found 683.70. ¹H NMR (300 MHz, Chloroform-d) δ 7.40-7.35 (m, 5H), 7.22-7.17 (m, 2H), 6.16 (s, 1H), 5.90 (s, 1H), 5.74 (s, 1H), 5.14 (s, 2H), 4.40-4.30 (m, 1H), 3.99-3.94 (m, 1H), 3.83-3.68 (m, 7H), 3.67-3.62 (m, 1H), 3.51-3.43 (m, 7H), 3.23-3.09 (m, 2H), 2.56-2.50 (m, 1H), 2.30-2.23 (m, 5H), 1.77-1.73 (m, 2H).

Preparations 135F/136F: Benzyl ((3R,4R)-4-hydroxy-1-(4-(2-methyl-5-((S)-3-(2,2,2-trifluoroethyl)pyrrolidine-1-carboxamido)phenyl)-6-morpholinopyridin-2-yl)pyrrolidin-3-yl)carbamate and benzyl ((3S,4S)-4-hydroxy-1-(4-(2-methyl-5-((S)-3-(2,2,2-trifluoroethyl)pyrrolidine-1-carboxamido)phenyl)-6-morpholinopyridin-2-yl)pyrrolidin-3-yl)carbamate

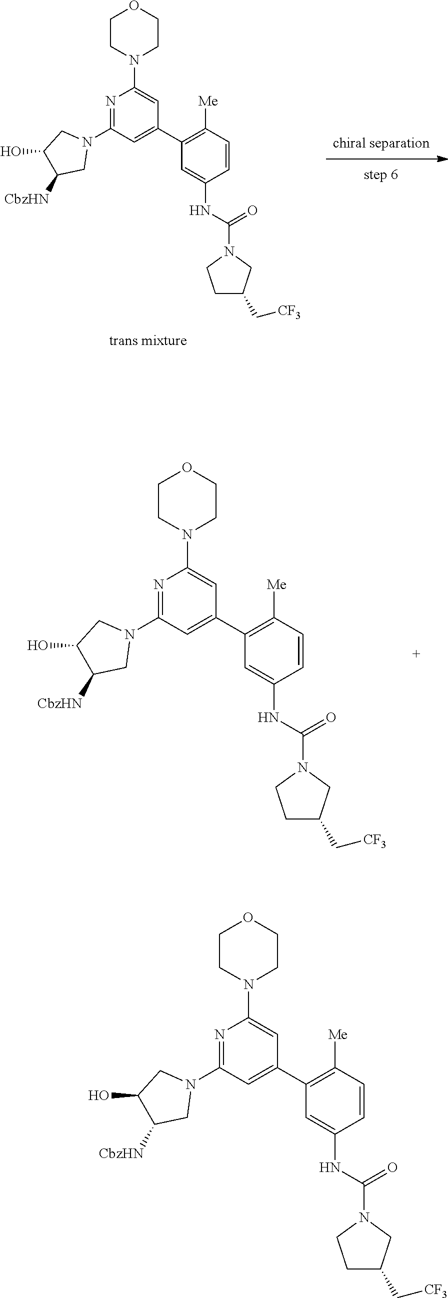 [SMILES: Cc1ccc(NC(=O)N2CC[C@@H](CC(F)(F)F)C2)cc1-c1cc(N2CCOCC2)nc(N2C[C@@H](O)[C@H](NC(=O)OCc3ccccc3)C2)c1 ; Cc1ccc(NC(=O)N2CC[C@@H](CC(F)(F)F)C2)cc1-c1cc(N2CCOCC2)nc(N2C[C@@H](O)[C@H](NC(=O)OCc3ccccc3)C2)c1 ; Cc1ccc(NC(=O)N2CC[C@@H](CC(F)(F)F)C2)cc1-c1cc(N2CCOCC2)nc(N2C[C@H](NC(=O)OCc3ccccc3)[C@@H](O)C2)c1]

Trans-benzyl (4-hydroxy-1-(4-(2-methyl-5-((S)-3-(2,2,2-trifluoroethyl)pyrrolidine-1-carboxamido)phenyl)-6-morpholinopyridin-2-yl)pyrrolidin-3-yl)carbamate (270 mg) was resolved by Chiral-Prep-HPLC with the following conditions: Column: (R,R)-WHELK-O1-Kromasil, 2.12×25 cm, 5 μm; Mobile Phase A: MTBE (0.5% 2M NH₃-MeOH), Mobile Phase B: EtOH; Flow rate:20 mL/min; Gradient: A:B=70:30; 220/254 nm. RT₁: 13.7 min gave 100 mg (35%) of Preparations 135F as an off-white solid, MS ESI calculated for C₃₅H₄₁F₃N₆O₅[M+H]⁺, 683.31, found 683.35.

And RT₂: 18.3 min gave 70 mg (24%) of Preparations 136F as an off-white solid. MS ESI calculated for C₃₅H₄₁F₃N₆O₅[M+H]⁺, 683.31, found 683.35.

### Examples 135 and 136: (3S)-N-(3-{2-[(3R,4R)-3-amino-4-hydroxypyrrolidin-1-yl]-6-(morpholin-4-yl)pyridin-4-yl}-4-methylphenyl)-3-(2,2,2-trifluoroethyl)pyrrolidine-1-carboxamide and (3S)-N-(3-{2-[(3S,4S)-3-amino-4-hydroxypyrrolidin-1-yl]-6-(morpholin-4-yl)pyridin-4-yl}-4-methylphenyl)-3-(2,2,2-trifluoroethyl)pyrrolidine-1-carboxamide

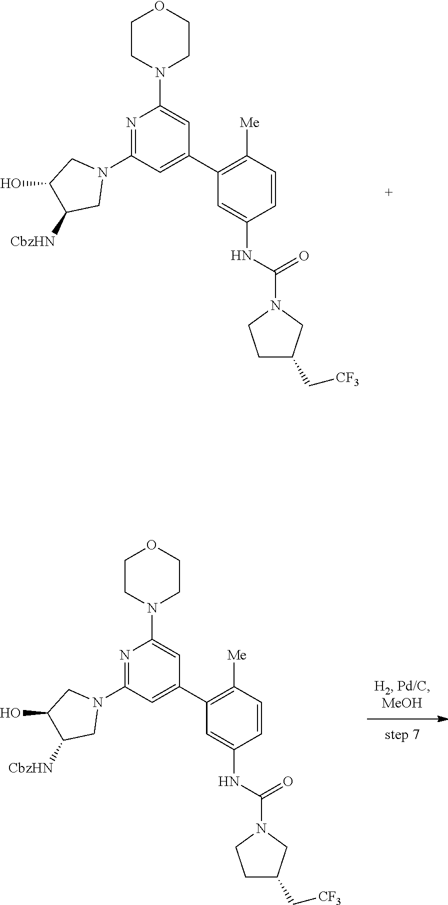 [SMILES: Cc1ccc(NC(=O)N2CC[C@@H](CC(F)(F)F)C2)cc1-c1cc(N2CCOCC2)nc(N2C[C@@H](O)[C@H](NC(=O)OCc3ccccc3)C2)c1 ; Cc1ccc(NC(=O)N2CC[C@@H](CC(F)(F)F)C2)cc1-c1cc(N2CCOCC2)nc(N2C[C@H](NC(=O)OCc3ccccc3)[C@@H](O)C2)c1]

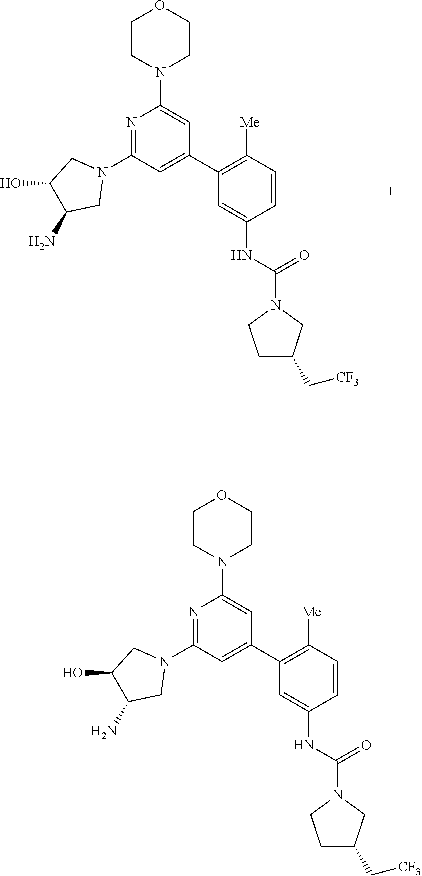 [SMILES: Cc1ccc(NC(=O)N2CC[C@@H](CC(F)(F)F)C2)cc1-c1cc(N2CCOCC2)nc(N2C[C@@H](N)[C@H](O)C2)c1 ; Cc1ccc(NC(=O)N2CC[C@@H](CC(F)(F)F)C2)cc1-c1cc(N2CCOCC2)nc(N2C[C@H](N)[C@@H](O)C2)c1]

A mixture of Preparations 135F (100 mg, 0.146 mmol) and Pd/C (50 mg, 0.470 mmol) in MeOH (2 mL) was stirred for 30 min at room temperature under H₂ atmosphere. The resulting mixture was filtered and washed with MeOH (3×5 mL). The filtrate was concentrated under reduced pressure to afford Example 135 (27 mg, 32%) as an off-white solid. MS ESI calculated for C₂₇H₃₅F₃N₆O₃[M+H]⁺, 549.27, found 549.25. ¹H NMR (300 MHz, DMSO-d₆) δ 8.12 (s, 1H), 7.51-7.35 (m, 2H), 7.16-7.10 (m, 1H), 5.89-5.84 (m, 1H), 5.66-5.61 (m, 1H), 5.00 (s, 1H), 3.90-3.86 (m, 1H), 3.69-3.51 (m, 8H), 3.42-3.33 (m, 4H), 3.28-3.13 (m, 3H), 3.09-2.94 (m, 2H), 2.74-2.47 (m, 3H), 2.29 (s, 3H), 2.18-2.02 (m, 1H), 1.72-1.58 (m, 2H). ¹⁹F NMR (282 MHz, DMSO-d₆) δ-63.35 (3F).

A mixture of Preparations 136F (75 mg, 0.110 mmol) and Pd/C (50 mg, 0.470 mmol) in MeOH (2 mL) was stirred for 30 min at room temperature under H₂ atmosphere. The resulting mixture was filtered and washed with MeOH (3×5 mL). The filtrate was concentrated under reduced pressure to afford Example 136 (29 mg, 46%) as an off-white solid. MS ESI calculated for C₂₇H₃₅F₃N₆O₃[M+H]⁺, 549.27, found 549.25. ¹H NMR (400 MHz, DMSO-d₆) δ 8.11 (s, 1H), 7.42 (dd, J=2.4, 8.4 Hz, 1H), 7.33 (d, J=2.0 Hz, 1H), 7.09 (d, J=8.4 Hz, 1H), 5.82 (s, 1H), 5.60 (s, 1H), 4.99 (s, 1H), 3.86 (s, 1H), 3.68-3.60 (m, 6H), 3.59-3.53 (m, 2H), 3.50-3.39 (m, 4H), 3.32-3.29 (m, 1H), 3.25-3.19 (m, 2H), 3.12-2.99 (m, 2H), 2.52-2.43 (m, 3H), 2.16 (s, 3H), 2.09-2.08 (m, 1H), 1.75-1.55 (m, 2H). ¹⁹F NMR (376 MHz, DMSO-d₆) δ-63.37 (3F).

### Example 137: (S)-N-(4-methyl-3-(2-(((R)-1-(methylamino)-1-oxopropan-2-yl)amino)-6-morpholinopyridin-4-yl)phenyl)-3-(2,2,2-trifluoroethyl)pyrrolidine-1-carboxamide

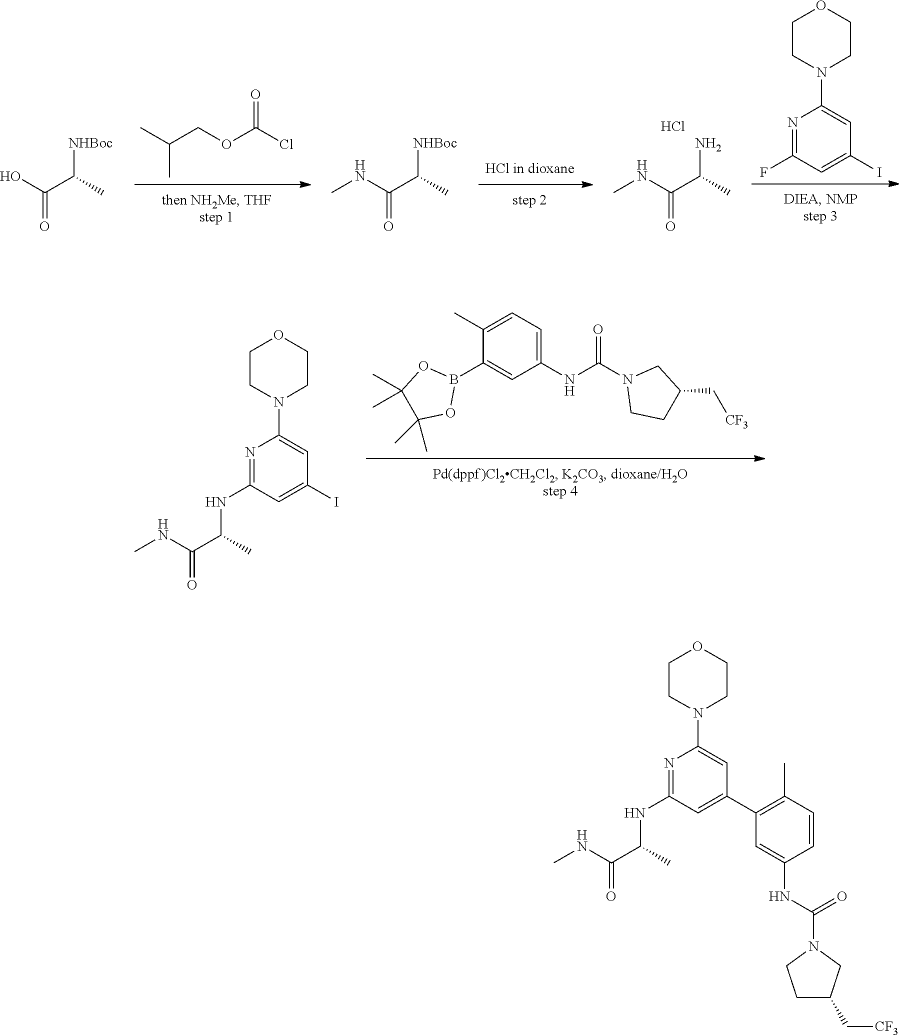

Preparation 137A: tert-butyl N-[(1R)-1-(methylcarbamoyl)ethyl]carbamate

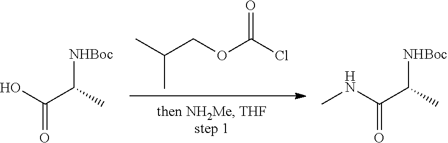

To a stirred solution of (tert-butoxycarbonyl)-D-alanine (3.00 g, 15.86 mmol) in THF (30 mL) was added isobutyl carbonochloridate (2.26 mL, 17.45 mmol) and N-methyl morphofine (1.9 mL, 17.45 mmol) at 0° C. The resulting mixture was stirred for 1 h at 0° C. To this was added methylamine (16.8 mL, 79.30 mmol). The resulting mixture was stirred for 1 h at 0° C. under nitrogen atmosphere.The reaction was quenched by the addition of water (30 mL). The resulting mixture was concentrated under reduced pressure to afford tert-butyl N-[(1R)-1-(methylcarbamoyl)ethyl]carbamate (3 g, crude) as a light yellow solid. C₉H₁₈N₂O₃, ¹H NMR (400 MHz, Chloroform-d) δ 3.74-3.70 (m, 1H), 3.23-3.19 (m, 1H), 2.80 (s, 3H), 1.44 (s, 9H), 1.35 (d, J=7.2 Hz, 3H).

Preparation 137B: (R)-2-amino-N-methylpropanamide hydrochloride

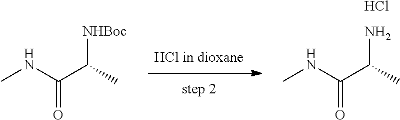

To a solution of tert-butyl N-[(1R)-1-(methylcarbamoyl)ethyl]carbamate (680 mg, 3.360 mmol) in 1,4-dioxane (15 mL) was added HCl (4 M in dioxane, 4 mL). The reaction mixture was stirred for 1 h at room temperature. The precipitated solids were collected by filtration and washed with diethyl ether (3×50 mL) to afford (R)-2-amino-N-methylpropanamide hydrochloride (630 mg, 95%). C₄H₁₁ClN₂O, ¹H NMR (300 MHz, DMSO-d₆) δ 8.57 (brs, 1H), 8.29 (brs, 3H), 3.82-3.76 (m, 1H), 2.67-3.73 (m, 3H), 1.35 (d, J=7.2 Hz, 3H).

Preparation 137C: (2R)-2-{[4-iodo-6-(morpholin-4-yl)pyridin-2-yl]amino}-N-methylpropanamide

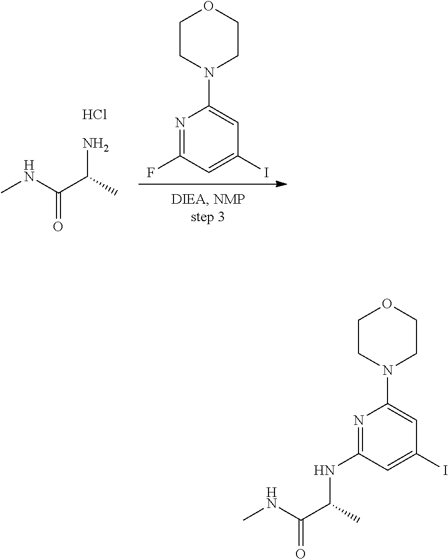

A solution of (R)-2-amino-N-methylpropanamide hydrochloride (315 mg, 2.270 mmol), 4-(6-fluoro-4-iodopyridin-2-yl)morpholine (350 mg, 1.140 mmol), NMP (5 mL) and DIEA (0.86 mL, 4.45 mmol) was stirred for 16 h at 160° C. The reaction was quenched with water (30 mL). The residue was extracted with EtOAc (3×30 mL). The resulting mixture was concentrated under reduced pressure. The residue was purified by silica gel column chromatography, eluted with PE/EtOAc (1/1) to afford (2R)-2-{[4-iodo-6-(morpholin-4-yl)pyridin-2-yl]amino}-N-methylpropanamide (151 mg, 34%) as a brown solid. MS ESI calculated for C₁₃H₁₉IN₄O₂[M+H]⁺, 391.06, found 391.00. ¹H NMR (300 MHz, Chloroform-d) δ 6.42 (brs, 1H), 6.35 (s, 1H), 6.21 (s, 1H), 4.44-4.40 (m, 1H), 4.19-4.15 (m, 1H), 3.79-3.75 (m, 4H), 3.43-3.40 (m, 4H), 2.78 (d, J=4.8 Hz, 3H), 1.46 (d, J=7.2 Hz, 3H).

### Example 137: (S)-N-(4-methyl-3-(2-(((R)-1-(methylamino)-1-oxopropan-2-yl)amino)-6-morpholinopyridin-4-yl)phenyl)-3-(2,2,2-trifluoroethyl)pyrrolidine-1-carboxamide

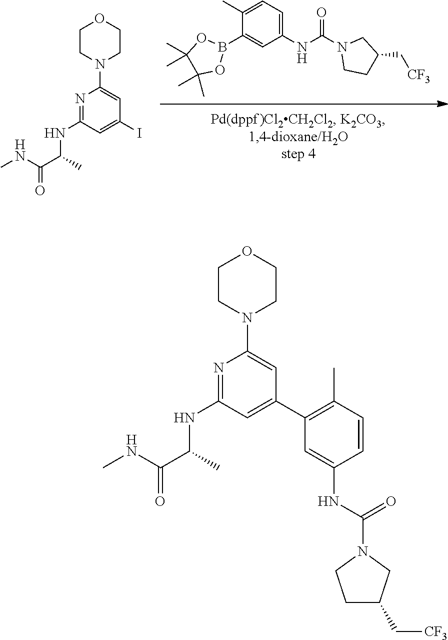 [SMILES: CNC(=O)[C@@H](C)Nc1cc(I)cc(N2CCOCC2)n1 ; Cc1ccc(NC(=O)N2CC[C@@H](CC(F)(F)F)C2)cc1B1OC(C)(C)C(C)(C)O1 ; CNC(=O)[C@@H](C)Nc1cc(-c2cc(NC(=O)N3CC[C@@H](CC(F)(F)F)C3)ccc2C)cc(N2CCOCC2)n1]

A mixture of (2R)-2-{[4-iodo-6-(morpholin-4-yl)pyridin-2-yl]amino}-N-methylpropanamide (100 mg, 0.260 mmol), (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (127 mg, 0.310 mmol), 1,4-dioxane (2 mL), H₂O (0.5 mL), Pd(dppf)Cl₂·CH₂Cl₂ (31 mg, 0.026 mmol) and K₂CO₃ (106 mg, 0.770 mmol) was stirred for 2 h at 80° C. under nitrogen atmosphere. The reaction was quenched by the addition of water (10 mL). The resulting mixture was concentrated under reduced pressure. The residue was purified by silica gel column chromatography, eluted with EtOAc. The crude product was resolved by Chiral-Prep-HPLC with the following conditions (Column: CHIRALPAK AD-H, 2×25 cm, 5 μm; Mobile Phase A: Hexane (0.5% 2M NH₃-MeOH), Mobile Phase B: IPA; Flow rate:20 mL/min; Gradient: A:B=70:30; 220/254 nm; RT₁: 12.12 min; to afford (S)-N-(4-methyl-3-(2-(((S)-1-(methylamino)-1-oxopropan-2-yl)amino)-6-morpholinopyridin-4-yl)phenyl)-3-(2,2,2-trifluoroethyl)pyrrolidine-1-carboxamide (13.9 mg, 9%) as an white solid and RT₂: 18.73 min afforded (S)-N-(4-methyl-3-(2-(((R)-1-(methylamino)-1-oxopropan-2-yl)amino)-6-morpholinopyridin-4-yl)phenyl)-3-(2,2,2-trifluoroethyl)pyrrolidine-1-carboxamide (124.7 mg, 87%) as a white solid. MS ESI calculated for C₂₇H₃₅F₃N₆O₃[M+H]⁺, 549.27, found 549.35. ¹H NMR (400 MHz, DMSO-d₆) δ 8.11 (s, 1H), 7.74-7.72 (m, 1H), 7.40-7.38 (m, 2H), 7.09 (d, J=8.4 Hz, 1H), 6.43 (d, J=6.4 Hz, 1H), 5.82 (s, 1H), 5.79 (s, 1H), 4.23-4.17 (m, 1H), 3.66-3.55 (m, 5H), 3.52-3.50 (m, 1H), 3.36-3.30 (m, 4H), 3.04-3.01 (m, 1H), 2.58-2.50 (m, 3H), 2.45-2.40 (m, 3H), 2.15 (s, 3H), 2.14-2.10 (m, 1H), 1.70-1.65 (m, 1H), 1.26 (d, J=7.2 Hz, 3H). ¹⁹F NMR (282 MHz, DMSO-d₆) δ-63.35 (3F).

### Example 138: (S)-N-(4-methyl-3-(2-(((S)-1-(methylamino)-1-oxopropan-2-yl)amino)-6-morpholinopyridin-4-yl)phenyl)-3-(2,2,2-trifluoroethyl)pyrrolidine-1-carboxamide

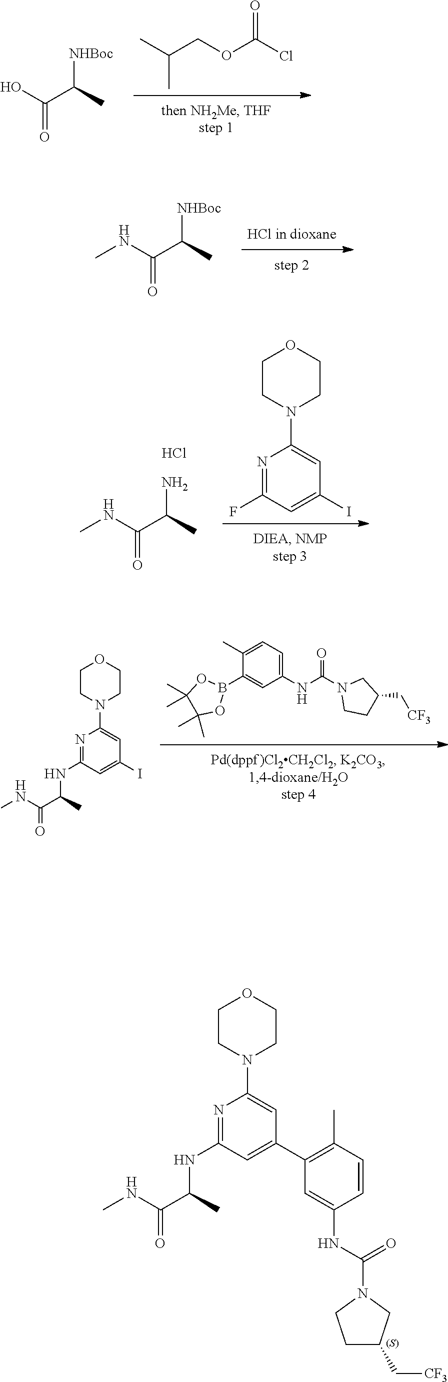

Preparation 138A: tert-butyl N-[(S)-1-(methylcarbamoyl)ethyl]carbamate

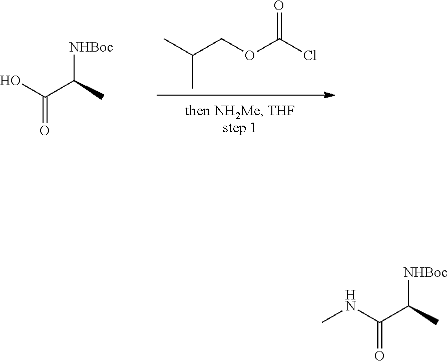

To a stirred solution of (tert-butoxycarbonyl)-L-alanine (2.00 g, 10.58 mmol) in THF (20 mL) was added isobutyl carbonochloridate (1.51 mL, 11.64 mmol) and N-methyl morphofine (1.28 mL, 11.64 mmol) at 0° C. for 1 h. The resulting mixture was stirred for 1.0 h at 0° C. under nitrogen atmosphere when methylamine (6.56 mL, 52.9 mmol) was added. The reaction was quenched by the addition of water (30 mL) at room temperature. The resulting mixture was concentrated under reduced pressure to afford tert-butyl N-[(S)-1-(methylcarbamoyl)ethyl]carbamate (2 g crude) as a light yellow solid.

Preparation138B: (S)-2-amino-N-methylpropanamide hydrochloride

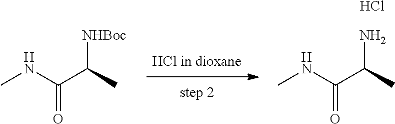

To a stirred solution of tert-butyl N-[(S)-1-(methylcarbamoyl)ethyl]carbamate (1.00 g, 4.94 mmol) in 1,4-dioxane were added HCl (4 M in dioxane, 5 mL)dropwise at room temperature under air atmosphere. The precipitated solids were collected by filtration and washed with diethyl ether (3×50 mL) to afford (S)-2-amino-N-methylpropanamide hydrochloride (920 mg, 95%) as a white solid.

Preparation 138C: (S)-2-{[4-iodo-6-(morpholin-4-yl)pyridin-2-yl]amino}-N-methylpropanamide

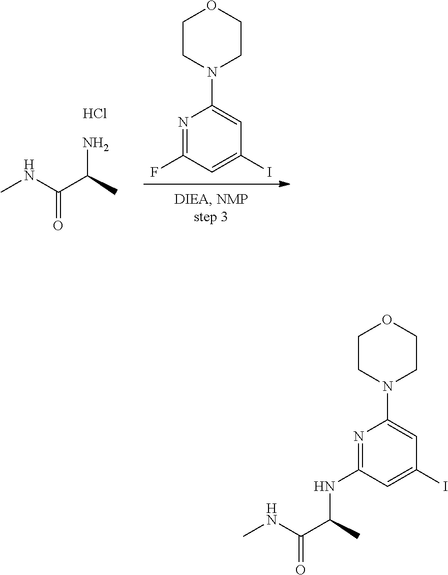

To a stirred solution of (S)-2-amino-N-methylpropanamide hydrochloride (450 mg, 3.246 mmol) and 4-(6-fluoro-4-iodopyridin-2-yl)morpholine (500 mg, 1.623 mmol) in NMP were added DIEA (839 mg, 6.492 mmol). The resulting mixture was stirred for 24 h at 170° C. The reaction was quenched with water (30 mL). The residue was washed with ethyl acetate (3×30 mL).The resulting mixture was concentrated under reduced pressure. The residue was purified by silica gel column chromatography, eluted with PE/EtOAc (1/1) to afford (S)-2-{[4-iodo-6-(morpholin-4-yl)pyridin-2-yl]amino}-N-methylpropanamide (204 mg, 32%) as a brown solid. MS ESI calculated for C₁₃H₁₉IN₄O₂[M+H]⁺, 391.06, found 391.00.

### Example 138: (S)-N-(4-methyl-3-(2-(((S)-1-(methylamino)-1-oxopropan-2-yl)amino)-6-morpholinopyridin-4-yl)phenyl)-3-(2,2,2-trifluoroethyl)pyrrolidine-1-carboxamide

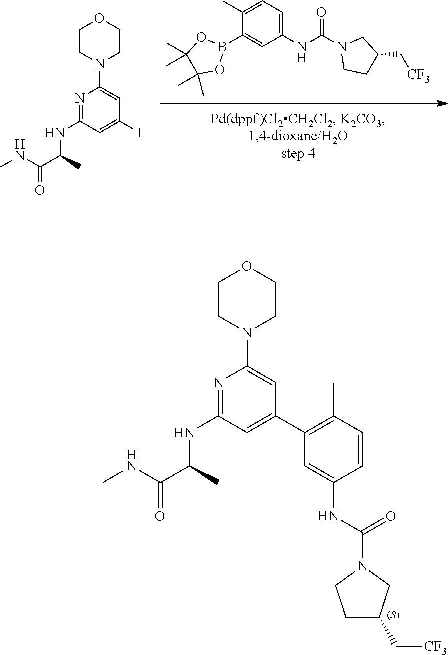

To a stirred solution/mixture of (2S)-2-{[4-iodo-6-(morpholin-4-yl)pyridin-2-yl]amino}-N-methylpropanamide (117 mg, 0.300 mmol) and (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (123.61 mg, 0.300 mmol) in 1,4-dioxane and H₂O were added Pd(dppf)Cl₂·CH₂Cl₂ (26.3 mg, 0.032 mmol) and K₂CO₃ (124.31 mg, 0.900 mmol). The resulting mixture was stirred for 2 h at 80° C. under nitrogen atmosphere. The resulting mixture was concentrated under reduced pressure. The residue was purified by silica gel column chromatography, eluted with EtOAc. The crude was resolved by Chiral-Prep-HPLC with the following conditions (Column: CHIRALPAK AD-H, 2×25 cm, 5 μm; Mobile Phase A: Hexane (0.5% 2M NH₃-MeOH), Mobile Phase B: IPA; Flow rate:20 mL/min; Gradient: A:B=70:30; 220/254 nm; RT₁: 12.12 min to afford (S)-N-(4-methyl-3-(2-(((S)-1-(methylamino)-1-oxopropan-2-yl)amino)-6-morpholinopyridin-4-yl)phenyl)-3-(2,2,2-trifluoroethyl)pyrrolidine-1-carboxamide (51 mg, 31%) as an white solid. MS ESI calculated for C₂₇H₃₅F₃N₆O₃[M+H]⁺, 549.27, found 549.20. ¹H NMR (400 MHz, DMSO-d₆) δ 8.11 (s, 1H), 7.74-7.72 (m, 1H), 7.40-7.38 (m, 2H), 7.09 (d, J=8.4 Hz, 1H), 6.43 (d, J=6.0 Hz, 1H), 5.82 (s, 1H), 5.79 (s, 1H), 4.23-4.17 (m, 1H), 3.66-3.55 (m, 5H), 3.52-3.50 (m, 1H), 3.36-3.28 (m, 4H), 3.04-3.01 (m, 1H), 2.58-2.50 (m, 3H), 2.45-2.40 (m, 4H), 2.15 (s, 3H), 2.14-2.10 (m, 1H), 1.70-1.65 (m, 1H), 1.26 (d, J=7.2 Hz, 3H). ¹⁹F NMR (282 MHz, DMSO-d₆) δ-63.35(3F). And RT₂: 18.73 min afforded (S)-N-(4-methyl-3-(2-(((R)-1-(methylamino)-1-oxopropan-2-yl)amino)-6-morpholinopyridin-4-yl)phenyl)-3-(2,2,2-trifluoroethyl)pyrrolidine-1-carboxamide (5.7 mg, 4%) as a white solid.

### Example 139: (3S)-N-{3-[2-ethanesulfonamido-6-(morpholin-4-yl)pyridin-4-yl]-4-methylphenyl}-3-(2,2,2-trifluoroethyl)pyrrolidine-1-carboxamide

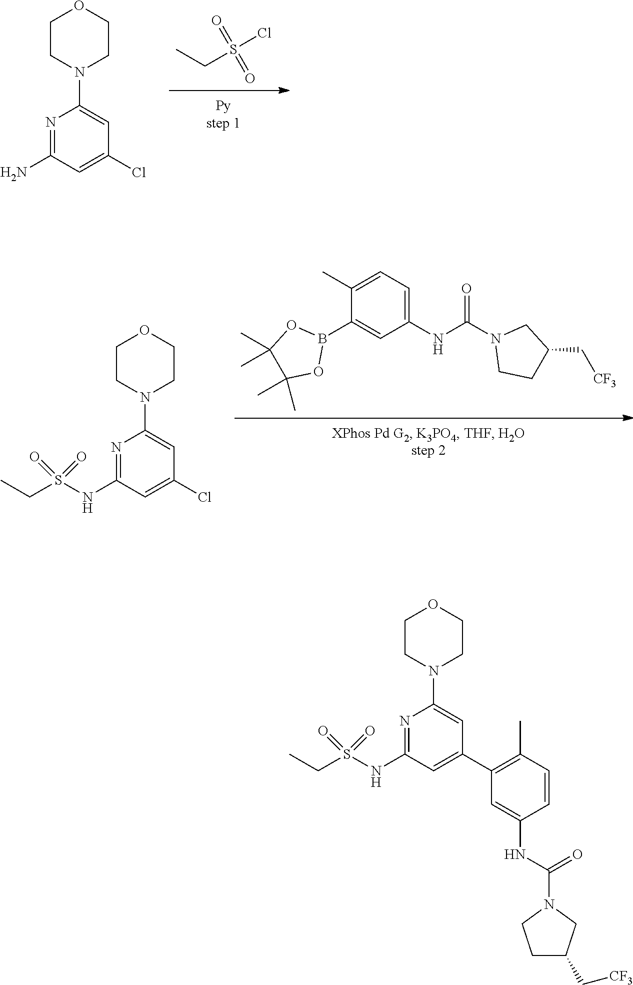

Preparation 139A: N-[4-chloro-6-(morpholin-4-yl)pyridin-2-yl]ethanesulfonamide

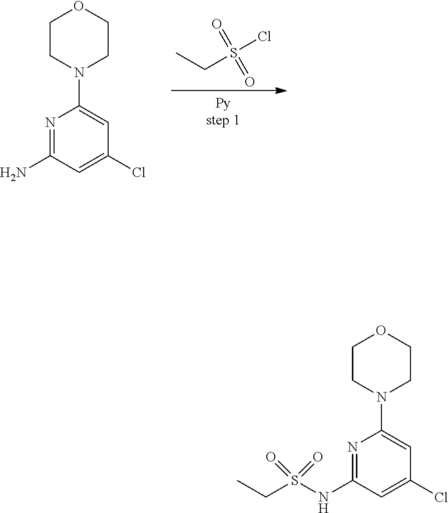

A mixture of 4-chloro-6-(morpholin-4-yl)pyridin-2-amine (500 mg, 2.340 mmol) and ethanesulfonyl chloride (451 mg, 3.510 mmol) in pyridine (7.5 mL) was stirred for 16 h at 80° C. The resulting mixture was concentrated under reduced pressure. The residue was purified by silica gel column chromatography, eluted with PE/EtOAc (1/1) to afford N-[4-chloro-6-(morpholin-4-yl)pyridin-2-yl]ethanesulfonamide (120 mg, 15%) as a brown solid. MS ESI calculated for C₁₁H₁₆ClN₃O₃S [M+H]⁺, 306.06, found 306.10. ¹H NMR (400 MHz, Chloroform-d) δ 6.84 (brs, 1H), 6.51 (s, 1H), 6.31 (s, 1H), 3.80-3.77 (m, 4H), 3.48-3.46 (m, 4H), 3.35 (q, J=7.2 Hz, 2H), 1.41 (t, J=7.2 Hz, 3H).

### Example 139: (3S)-N-{3-[2-ethanesulfonamido-6-(morpholin-4-yl)pyridin-4-yl]-4-methylphenyl}-3-(2,2,2-trifluoroethyl)pyrrolidine-1-carboxamide

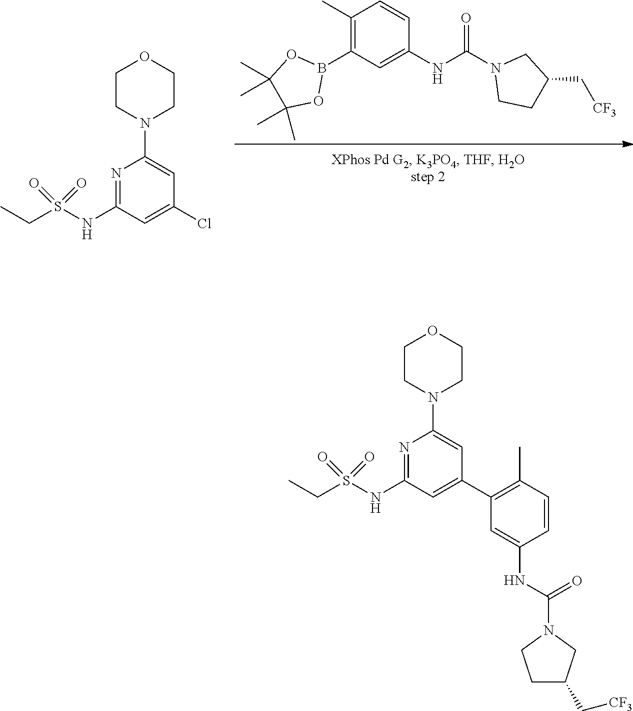 [SMILES: CCS(=O)(=O)Nc1cc(Cl)cc(N2CCOCC2)n1 ; Cc1ccc(NC(=O)N2CC[C@@H](CC(F)(F)F)C2)cc1B1OC(C)(C)C(C)(C)O1 ; CCS(=O)(=O)Nc1cc(-c2cc(NC(=O)N3CC[C@@H](CC(F)(F)F)C3)ccc2C)cc(N2CCOCC2)n1]

A mixture of N-[4-chloro-6-(morpholin-4-yl)pyridin-2-yl]ethanesulfonamide (100 mg, 0.327 mmol), (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (108 mg, 0.262 mmol), K₃PO₄ (139 mg, 0.654 mmol) and 2^{nd} XPhos Precatalyst (26 mg, 0.033 mmol) in THF (1 mL) and H₂O (0.1 mL) was stirred for 2 h at 80° C. under nitrogen atmosphere. The resulting mixture was concentrated under reduced pressure. The residue was purified by Prep-TLC with PE/EtOAc/EtOH (8/3/1) to afford (3S)-N-{3-[2-ethanesulfonamido-6-(morpholin-4-yl)pyridin-4-yl]-4-methylphenyl}-3-(2,2,2-trifluoroethyl)pyrrolidine-1-carboxamide (87 mg, 46%) as an off-white solid. MS ESI calculated for C₂₅H₃₂F₃N₅O₄S [M+H]⁺, 556.21, found 556.30. ¹H NMR (400 MHz, DMSO-d₆) δ 10.25 (brs, 1H), 8.16 (s, 1H), 7.46 (dd, J=2.4, 8.4 Hz, 1H), 7.36 (d, J=2.4 Hz, 1H), 7.14 (d, J=8.4 Hz, 1H), 6.32 (s, 1H), 6.15 (s, 1H), 3.71-3.65 (m, 5H), 3.53-3.43 (m, 7H), 3.32-3.30 (m, 1H), 3.02-3.00 (m, 1H), 2.53-2.33 (m, 3H), 2.20 (s, 3H), 2.12-2.00 (m, 1H), 2.75-2.65 (m, 1H), 1.26 (t, J=7.2 Hz, 3H). ¹⁹F NMR (376 MHz, DMSO-d₆) δ-63.37 (3F).

### Example 140: (3S)-N-{4-methyl-3-[2-(morpholin-4-yl)-6-(propane-2-sulfonamido)pyridin-4-yl]phenyl}-3-(2,2,2-trifluoroethyl)pyrrolidine-1-carboxamide

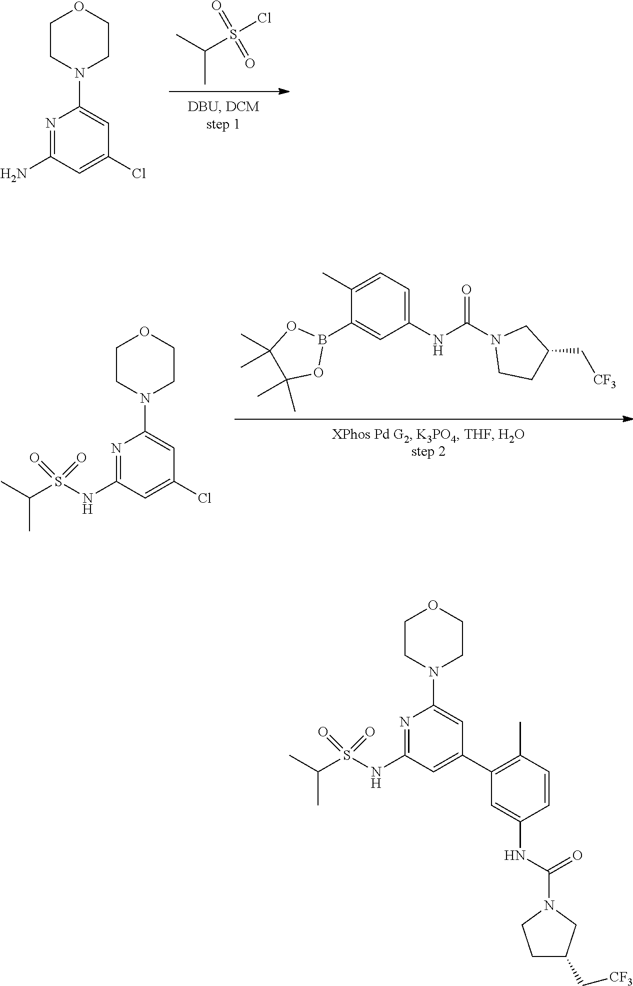

Preparation 140A: N-[4-chloro-6-(morpholin-4-yl)pyridin-2-yl]propane-2-sulfonamide

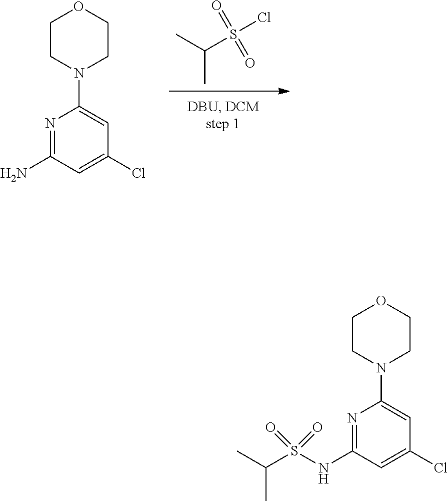

To a stirred mixture of 4-chloro-6-(morpholin-4-yl)pyridin-2-amine (500 mg, 2.340 mmol) and DBU (1.43 g, 9.393 mmol) in DCM (7 mL) was added propane-2-sulfonyl chloride (350 mg, 9.360 mmol) dropwise at −30° C. The reaction mixture was stirred for 3 h at −30° C. The resulting mixture was diluted with water (30 mL). The resulting mixture was extracted with DCM (3×20 mL). The combined organic layers were washed with brine (2×20 mL), dried over anhydrous Na₂SO₄. After filtration, the filtrate was concentrated under reduced pressure. The residue was purified by reverse phase chromatography, eluted with 40% CH₃CN in water (plus 0.5% TFA) to afford N-[4-chloro-6-(morpholin-4-yl)pyridin-2-yl]propane-2-sulfonamide (350 mg, 47%) as a light yellow solid. MS ESI calculated for C₁₂H₁₈ClN₃O₃S [M+H]⁺, 320.08; found 320.10. ¹H NMR (400 MHz, Chloroform-d) δ 6.62 (d, J=1.2 Hz, 1H), 6.33 (d, J=1.2 Hz, 1H), 3.84-3.77 (m, 4H), 3.62-3.45 (m, 5H), 1.45 (d, J=6.8 Hz, 6H).

### Example 140: (3S)-N-{4-methyl-3-[2-(morpholin-4-yl)-6-(propane-2-sulfonamido)pyridin-4-yl]phenyl}-3-(2,2,2-trifluoroethyl)pyrrolidine-1-carboxamide

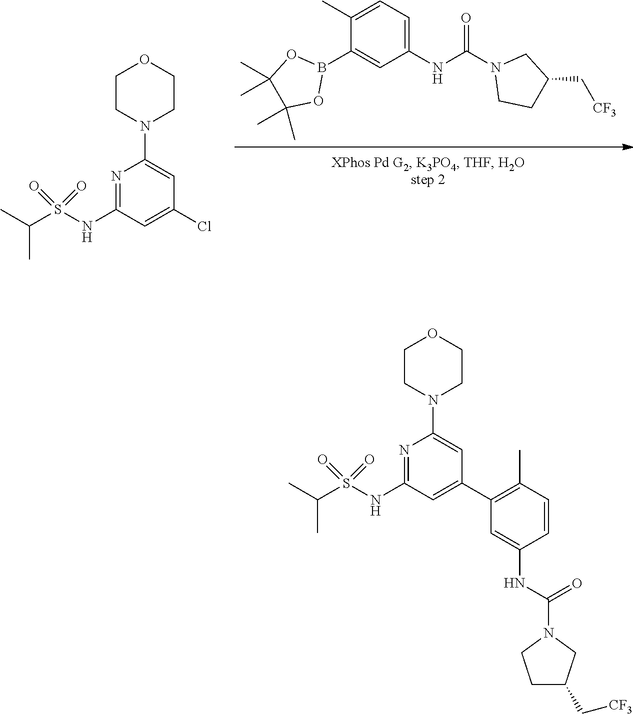 [SMILES: CC(C)S(=O)(=O)Nc1cc(Cl)cc(N2CCOCC2)n1 ; Cc1ccc(NC(=O)N2CC[C@@H](CC(F)(F)F)C2)cc1B1OC(C)(C)C(C)(C)O1 ; Cc1ccc(NC(=O)N2CC[C@@H](CC(F)(F)F)C2)cc1-c1cc(NS(=O)(=O)C(C)C)nc(N2CCOCC2)c1]

A mixture of N-[4-chloro-6-(morpholin-4-yl)pyridin-2-yl]propane-2-sulfonamide (120 mg, 0.375 mmol), K₃PO₄ (239 mg, 1.125 mmol), (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (155 mg, 0.375 mmol) in THF (2 mL) and H₂O (0.2 mL) was added 2^{nd} XPhos Precatalyst (29 mg, 0.038 mmol) was stirred for 2 h at 80° C. under nitrogen atmosphere. The resulting mixture was concentrated under reduced pressure. The residue was purified by silica gel column chromatography, eluted with PE/EtOAc (2/3) and reverse phase chromatography, eluted with 45% CH₃CN in water (0.05% NH₄HCO₃) to afford (3S)-N-{4-methyl-3-[2-(morpholin-4-yl)-6-(propane-2-sulfonamido)pyridin-4-yl]phenyl}-3-(2,2,2-trifluoroethyl)pyrrolidine-1-carboxamide (145.2 mg, 66%) as a white solid. MS ESI calculated for C₂₆H₃₄F₃N₅O₄S [M+H]⁺, 570.23; found 570.30.

¹H NMR (300 MHz, Chloroform-d) δ 7.33-7.30 (m, 2H), 7.19 (d, J=8.4 Hz, 1H), 6.58 (brs, 1H), 6.30 (s, 1H), 6.17 (s, 1H), 3.85-3.80 (m, 5H), 3.68-3.43 (m, 7H), 3.14-3.11 (m, 1H), 2.64-2.53 (m, 1H), 2.35-2.15 (m, 6H), 1.84-1.66 (m, 1H), 1.46 (d, J=6.9 Hz, 6H). ¹⁹F NMR (282 MHz, Chloroform-d) δ-64.95 (3F).

### Example 141: (3S)-N-{4-methyl-3-[2-(1-methylcyclopropanesulfonamido)-6-(morpholin-4-yl)pyridin-4-yl]phenyl}-3-(2,2,2-trifluoroethyl)pyrrolidine-1-carboxamide

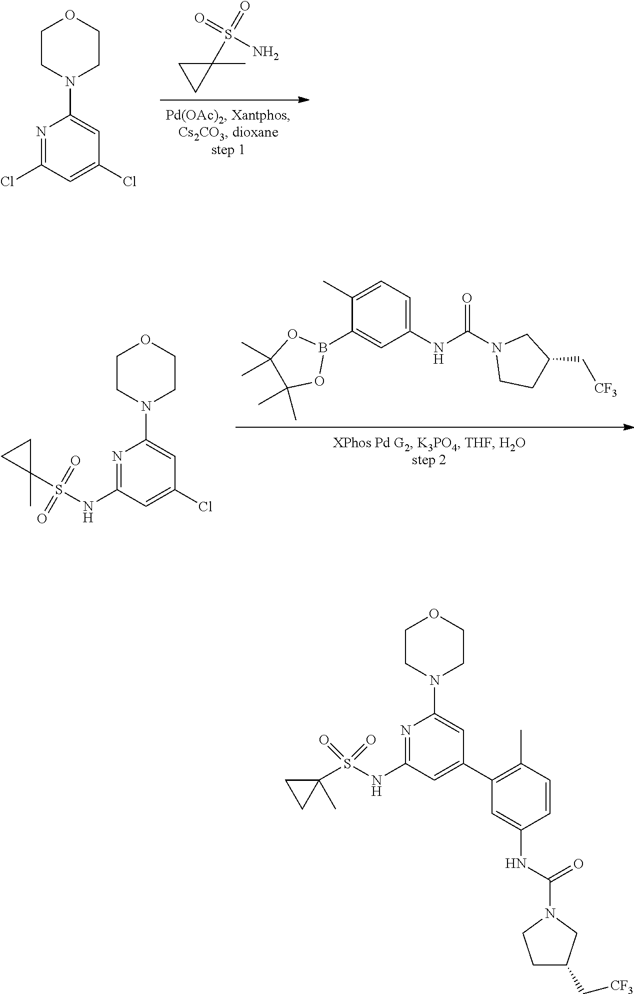

Preparation 141A: N-[4-chloro-6-(morpholin-4-yl)pyridin-2-yl]-1-methylcyclopropane-1-sulfonamide

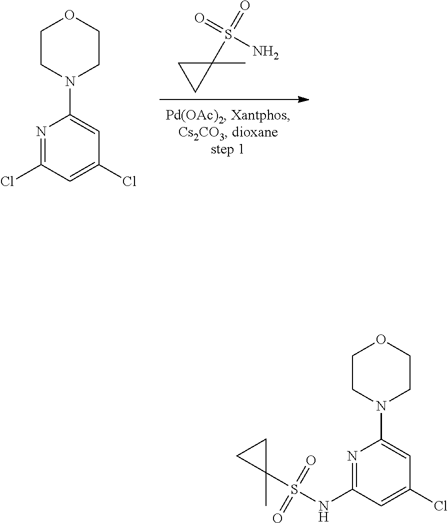

A mixture of 4-(4,6-dichloropyridin-2-yl)morpholine (1.00 g, 4.29 mmol), 1-methylcyclopropane-1-sulfonamide (0.58 g, 4.29 mmol), dioxane (12 mL), XantPhos (0.25 g, 0.43 mmol) Cs₂CO₃ (4.2 g, 12.87 mmol) and Pd(OAc)₂ (0.10 g, 0.43 mmol) was stirred for 16 h at 100° C. under nitrogen atmosphere. The resulting mixture was diluted with water (50 mL). The resulting mixture was extracted with EtOAc (3×30 mL). The combined organic layers were washed with brine (2×50 mL), dried over anhydrous Na₂SO₄. After filtration, the filtrate was concentrated under reduced pressure. The residue was purified by silica gel column chromatography, eluted with PE/EtOAc (1/1) to afford N-[4-chloro-6-(morpholin-4-yl)pyridin-2-yl]-1-methylcyclopropane-1-sulfonamide (1.00 g, 70%). MS ESI calculated for C₁₃H₁₈ClN₃O₃S [M+H]⁺, 332.08; found 332.10. ¹H NMR (400 MHz, Methanol-d₄) δ 11.11 (s, 1H), 7.34 (s, 1H), 7.20 (s, 1H), 4.49-4.47 (m, 4H), 4.29-4.27 (m, 4H), 2.23 (s, 3H), 2.14-2.03 (m, 2H), 1.71-1.63 (m, 2H).

### Example 141: (3S)-N-{4-methyl-3-[2-(1-methylcyclopropanesulfonamido)-6-(morpholin-4-yl)pyridin-4-yl]phenyl}-3-(2,2,2-trifluoroethyl)pyrrolidine-1-carboxamide

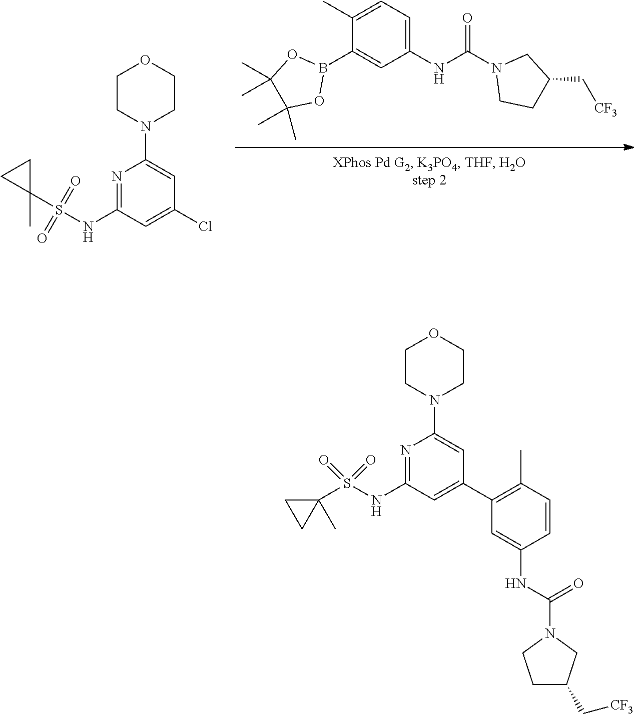 [SMILES: CC1(S(=O)(=O)Nc2cc(Cl)cc(N3CCOCC3)n2)CC1 ; Cc1ccc(NC(=O)N2CC[C@@H](CC(F)(F)F)C2)cc1B1OC(C)(C)C(C)(C)O1 ; Cc1ccc(NC(=O)N2CC[C@@H](CC(F)(F)F)C2)cc1-c1cc(NS(=O)(=O)C2(C)CC2)nc(N2CCOCC2)c1]

A mixture of N-[4-chloro-6-(morpholin-4-yl)pyridin-2-yl]-1-methylcyclopropane-1-sulfonamide (150 mg, 0.452 mmol), (3S)-N-[4-methyl-3-(4,4,5,5-tetramethyl-1,3,2-dioxaborolan-2-yl)phenyl]-3-(2,2,2-trifluoroethyl)pyrrolidine-1-carboxamide (186 mg, 0.452 mmol), THF (2 mL), H₂O (0.2 mL), K₃PO₄ (288 mg, 1.356 mmol) and 2^{nd} XPhos Precatalyst (35 mg, 0.045 mmol) was stirred for 2 h at 80° C. under nitrogen atmosphere. The resulting mixture was concentrated under reduced pressure. The residue was purified by silica gel column chromatography, eluted with PE/EtOAc/EtOH (4/3/1) and reverse phase chromatography, eluted with 55% CH₃CN in water (0.05% NH₄HCO₃) to afford (3S)-N-{4-methyl-3-[2-(1-methylcyclopropanesulfonamido)-6-(morpholin-4-yl)pyridin-4-yl]phenyl}-3-(2,2,2-trifluoroethyl)pyrrolidine-1-carboxamide (71 mg, 27%) as a white solid. MS ESI calculated for C₂₇H₃₄F₃N₅O₄S [M+H]⁺, 582.23; found 582.30. ¹H NMR (300 MHz, Chloroform-d) δ 7.35-7.29 (m, 2H), 7.19 (d, J=8.4 Hz, 1H), 6.71 (brs, 2H), 6.31 (s, 1H), 6.16 (s, 1H), 3.85-3.82 (m, 5H), 3.68-3.63 (m, 1H), 3.60-3.40 (m, 5H), 3.17-3.11 (m, 1H), 2.64-2.53 (m, 1H), 2.39-2.18 (m, 6H), 1.84-1.77 (m, 1H), 1.58 (s, 3H), 1.48-1.46 (m, 2H), 0.84-0.82 (m, 2H). ¹⁹F NMR (282 MHz, Chloroform-d) δ-64.95 (3F).

### II. Biological Evaluation

### Enzymatic RAFS Kinase Activity Determination:

Small molecule inhibition of RAF1 kinases was measured using ADP-Glo assay. In the assay, ADP is converted to ATP in the presence of test kinase and substrate, resulting in luciferase reaction and luminescent readout with light generated proportional to the relative kinase activity. Compounds diluted in DMSO were used in 10-point, 3-fold dose curve for both assays. Final concentrations of 3 nM RAF1 (CarnaBio, Cat. 09-125) and 30 nM MEK1 substrate (Millipore, Cat. 14-420) were incubated with 3 μM ATP, 10 mM MgCl2, 0.003% Brij-35, 2 mM DTT, 0.05% BSA, 1 mM EGTA, and 50 mM HEPES for 90 minutes at room temp prior to addition of ADP-Glo reagent (Promega, Cat. V9102) for 40 minutes, and detection reagent for 45 minutes. Luminescence was read on an Envision plate reader (PerkinElmer) and percent remaining activity was used to calculate IC50 using a four-parameter fit model using Dotmatics Knowledge Solutions Studies curve fitting (Dotmatics, Bishops Stortford, UK, CM23). Representative data for exemplary compounds is presented in Table 2.

TABLE 2

| Synthetic | |
|---|---|
| Chemistry | |
| Example | RAF-1 IC₅₀ |
| 1 | A |
| 2 | A |
| 3 | A |
| 4 | A |
| 5 | A |
| 6 | A |
| 7 | B |
| 8 | A |
| 9 | A |
| 10 | B |
| 11 | A |
| 12 | A |
| 13 | A |
| 14 | A |
| 15 | A |
| 16 | A |
| 17 | A |
| 18 | A |
| 19 | A |
| 20 | A |
| 21 | A |
| 22 | A |
| 23 | A |
| 24 | A |
| 25 | A |
| 26 | A |
| 27 | A |
| 28 | A |
| 29 | A |
| 30 | A |
| 31 | A |
| 32 | A |
| 33 | A |
| 34 | A |
| 35 | A |
| 36 | A |
| 37 | A |
| 38 | A |
| 39 | A |
| 40 | B |
| 41 | B |
| 42 | A |
| 43 | A |
| 44 | A |
| 45 | A |
| 46 | A |
| 47 | A |
| 48 | A |
| 49 | A |
| 50 | A |
| 51 | A |
| 52 | A |
| 53 | A |
| 54 | A |
| 55 | A |
| 56 | A |
| 57 | A |
| 58 | A |
| 59 | A |
| 60 | A |
| 61 | A |
| 62 | A |
| 63 | A |
| 64 | A |
| 65 | A |
| 66 | A |
| 67 | A |
| 68 | A |
| 69 | A |
| 70 | A |
| 71 | A |
| 72 | A |
| 73 | A |
| 74 | A |
| 75 | A |
| 76 | A |
| 77 | A |
| 78 | A |
| 79 | A |
| 80 | A |
| 81 | A |
| 82 | A |
| 83 | A |
| 84 | A |
| 85 | A |
| 86 | A |
| 87 | A |
| 88 | A |
| 89 | A |
| 90 | A |
| 91 | A |
| 92 | B |
| 93 | A |
| 94 | A |
| 95 | A |
| 96 | A |
| 97 | A |
| 98 | A |
| 99 | A |
| 100 | A |
| 101 | A |
| 102 | A |
| 103 | A |
| 104 | A |
| 105 | A |
| 106 | A |
| 107 | A |
| 108 | A |
| 109 | A |
| 110 | A |
| 111 | A |
| 112 | B |
| 113 | A |
| 114 | A |
| 115 | A |
| 116 | A |
| 117 | A |
| 118 | A |
| 119 | A |
| 120 | A |
| 121 | A |
| 122 | A |
| 123 | A |
| 124 | A |
| 125 | A |
| 126 | A |
| 127 | A |
| 128 | A |
| 129 | A |
| 130 | A |
| 131 | A |
| 132 | A |
| 133 | A |
| 134 | A |
| 135 | A |
| 136 | A |
| 137 | A |
| 138 | A |
| 139 | A |
| 140 | A |

Note:
Biochemical assay IC₅₀ data are designated within the following ranges:
A: ≤0.010 μM
B: >0.010 μM to ≤0.100 μM
C: >0.100 μM to ≤1 μM

### III. Preparation of Pharmaceutical Dosage Forms

### Example 1: Oral Capsule

The active ingredient is a compound of Table 1, or a pharmaceutically acceptable salt or solvate thereof. A capsule for oral administration is prepared by mixing 1-1000 mg of active ingredient with starch or other suitable powder blend. The mixture is incorporated into an oral dosage unit such as a hard gelatin capsule, which is suitable for oral administration.

### Example 2: Solution for Injection

The active ingredient is a compound of Table 1, or a pharmaceutically acceptable salt thereof, and is formulated as a solution in sesame oil at a concentration of 50 mg-eq/mL.

The examples and embodiments described herein are for illustrative purposes only and various modifications or changes suggested to persons skilled in the art are to be included within the spirit and purview of this application and scope of the appended claims.

1-68. (canceled)

69. A compound, or pharmaceutically acceptable salt or solvate thereof, having the structure of Formula (V):
(V)
wherein,
ring A is a 5- or 6-membered optionally substituted heteroaryl, or 5- or 6-membered optionally substituted heterocyclyl;
W is NH, or NR7;
X is N, C—H, C—F, or C—CN;
Y is N, C—F, or C—H;
R is H, C1-C8 optionally substituted alkyl, (C1-C8 optionally substituted alkylene)-OPO(OH)₂, C3-C6 optionally substituted cycloalkyl, (C3-C6 optionally substituted cycloalkylene)-OPO(OH)₂, C4-C8 optionally substituted cycloalkylalkyl, (C3-C6 optionally substituted cycloalkylalkylene)-OPO(OH)₂, C3-C6 optionally substituted heterocyclyl, (C3-C6 optionally substituted heterocyclyl)-OPO(OH)₂, C3-C6 optionally substituted heterocyclylalkyl, (C3-C6 optionally substituted heterocyclylalkyl)-OPO(OH)2, C1-C8 optionally substituted alkyl-CO—, SO2—(C1-C8 optionally substituted alkyl), —SO2—(C3-C6 optionally substituted cycloalkyl), —SO2—(C4-C8 optionally substituted cycloalkylalkyl), —SO2—(C3-C6 optionally substituted heterocyclyl), —SO2—(C3-C6 optionally substituted heterocyclylalkyl), —SO2-N(R8)R9-C1-C8 optionally substituted alkyl, -C02-C1-C8 optionally substituted alkyl, or —CO—N(R8)R9-C1-C8 optionally substituted alkyl;
each R8 and R9 is independently selected from H or C1-C8 optionally substituted alkyl;
R2 is H, D or F;
R4 is halogen, optionally substituted C1-C3 alkyl, -CD3, or optionally substituted C1-C3 alkoxy;
R6 is H, D, Cl or F;
R7 is C1-C8 optionally substituted alkyl; or R is not H, and R and R7 optionally join to form an optionally substituted heterocyclyl ring;
Z is (a) wherein m is 0, 1, 2, or 3; p is 0, 1, 2, 3, or 4; and each R11 is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —S-alkyl, optionally substituted —SO2alkyl, optionally substituted C3-C6 cycloalkylalkyl, optionally substituted heterocyclyl, or optionally substituted heterocyclylalkyl; or two RI 1 groups together form an oxo; or
(b) wherein m is 0, 1, 2, or 3; n is 0, 1, 2, or 3; p is 0, 1, 2, 3, or 4; and each R11 is independently selected from amino, alkylamino, dialkylamino, —OH, halogen, optionally substituted C1-C6 alkyl, optionally substituted C3-C6 cycloalkyl, optionally substituted C1-C6 alkoxy, optionally substituted C2-C6 alkynyl, optionally substituted —S-alkyl, optionally substituted —SO2alkyl, optionally substituted C3-C6 cycloalkylalkyl, optionally substituted heterocyclyl, or optionally substituted heterocyclylalkyl; or two RI 1 groups together form an oxo.

70. The compound of claim 69, or pharmaceutically acceptable salt or solvate thereof, wherein ring A is a 5- or 6-membered optionally substituted heteroaryl.

71. The compound of claim 69, or pharmaceutically acceptable salt or solvate thereof, wherein ring A is an optionally substituted morpholine.

72. The compound of claim 69, or pharmaceutically acceptable salt or solvate thereof, wherein W is NH.

73. The compound of claim 69, or pharmaceutically acceptable salt or solvate thereof, wherein W is NR7.

74. The compound of claim 69, or pharmaceutically acceptable salt or solvate thereof, wherein W is NR7, R is not H, and R and R7 join to form an optionally substituted heterocyclyl ring.

75. The compound of claim 69, or pharmaceutically acceptable salt or solvate thereof wherein R is SO2—(C1-C8 optionally substituted alkyl), —SO2—(C3-C6 optionally substituted cycloalkyl), —SO2—(C4-C8 optionally substituted cycloalkylalkyl), —SO2—(C3-C6 optionally substituted heterocyclyl), —SO2—(C3-C6 optionally substituted heterocyclylalkyl), —SO2—N(R8)R9-C1-C8 optionally substituted alkyl, —CO2-C1-C8 optionally substituted alkyl, or —CO—N(R8)R9-C1-C8 optionally substituted alkyl;
each R8 and R9 is independently selected from H or C1-C8 optionally substituted alkyl.

76. The compound of claim 69, or pharmaceutically acceptable salt or solvate thereof wherein R is C1-C8 optionally substituted alkylene)-OPO(OH)₂, C3-C6 optionally substituted cycloalkyl, (C3-C6 optionally substituted cycloalkylene)-OPO(OH)₂, C4-C8 optionally substituted cycloalkylalkyl, (C3-C6 optionally substituted cycloalkylalkylene)-OPO(OH)₂, C3-C6 optionally substituted heterocyclyl, (C3-C6 optionally substituted heterocyclyl)-OPO(OH)₂, C3-C6 optionally substituted heterocyclylalkyl, (C3-C6 optionally substituted heterocyclylalkyl)-OPO(OH)₂, C1-C8 optionally substituted alkyl-CO—.

77. A pharmaceutical composition comprising a compound of formula (V) or a pharmaceutically acceptable salt or solvate thereof as described in claim 69 and a pharmaceutically acceptable excipient.

78. The pharmaceutical composition of claim 77 formulated for oral administration or for administration by injection.

79. A method of preparing a pharmaceutical composition comprising mixing a compound, or pharmaceutically acceptable salt or solvate thereof, of claim 69, and a pharmaceutically acceptable carrier.

80. A method of treating cancer in a patient in need thereof comprising administering to the patient a compound of Formula (V), or a pharmaceutically acceptable salt or solvate thereof as described in claim 69.

81. A method of treating cancer in a patient in need thereof comprising administering to the patient a pharmaceutical composition as described in claim 77.

82. The method of treating cancer of claim 81 wherein the pharmaceutical composition is administered orally or by injection.

83. The method of treating cancer of claim 81 wherein the pharmaceutical composition is administered orally and wherein the oral dose ranges from 1.0 mg to about 1000 mg, one to four times or more, per day.
